(19) United States
(12) Patent Application Publication
(10) Pub. No.: US 2008/0034453 A1
(43) Pub. Date: Feb. 7, 2008

(54) Annotated Plant Genes

(76) Inventors: Nordine Cheikh, Chesterfield, MO (US); Jingdong Liu, Chesterfield, MO (US)

Correspondence Address: ARNOLD & PORTER, LLP, 555 TWELFTH STREET, N.W., ATTN: IP DOCKETING, WASHINGTON, DC, 20004, UNITED STATES, 202-942-5000, IP_DOCKETING@APORTER.COM

(21) Appl. No.: 09/371,146

(22) Filed: Aug. 9, 1999

Related U.S. Application Data
Application No. 09/371,146 is a continuation-in-part of application No. 09/304,517, filed on May 6, 1999, now abandoned.

(51) Int. Cl. A01H 1/00; C07H 21/04

(52) U.S. Cl. 800/294; 536/23.2

(57) ABSTRACT

The present invention is in the field of plant biochemistry. More specifically the invention relates to nucleic acid sequences from plant cells, in particular, nucleic acid sequences from maize and soybean. The invention encompasses nucleic acid molecules that encode proteins and fragments of proteins. In addition, the invention also encompasses proteins and fragments of proteins so encoded and antibodies capable of binding these proteins or fragments. The invention also relates to methods of using the nucleic acid molecules, proteins and fragments of proteins, and antibodies, for example for genome mapping, gene identification and analysis, plant breeding, preparation of constructs for use in plant gene expression, and transgenic plants.

### CROSS REFERENCE TO RELATED APPLICATION

The present application is a continuation-in-part of application Ser. No. 09/304,517, entitled “Annotated Plant Genes,” filed May 6, 1999.

### FIELD OF THE INVENTION

The present invention is in the field of plant biochemistry. More specifically the invention relates to nucleic acid sequences from plant cells, in particular, nucleic acid sequences from maize and soybean. The invention encompasses nucleic acid molecules that encode proteins and fragments of proteins. In addition, the invention also encompasses proteins and fragments of proteins so encoded and antibodies capable of binding these proteins or fragments. The invention also relates to methods of using the nucleic acid molecules, proteins and fragments of proteins, and antibodies, for example for genome mapping, gene identification and analysis, plant breeding, preparation of constructs for use in plant gene expression, and transgenic plants.

### BACKGROUND OF THE INVENTION

The present invention is directed in part to aspects of plant biochemistry. Plants exhibit differences in their biochemistry to non-plants (see, for example, Plant Biochemistry, Eds. Dey and Harborne, Academic Press, New York (1997)). Plants also exhibit similarities in their biochemistry with non-plants (see, for example, Plant Biochemistry, Eds. Dey and Harborne, Academic Press, New York (1997); Biochemistry, Stryer, 4^{th} Edition, W.H. Freeman & Co., New York (1995); Principles of Biochemistry, Lehniger et al., Worth Publishing, New York (1994)). In addition, difference plants can exhibit differences in their biochemistry (see, for example, Plant Biochemistry, Eds. Dey and Harborne, Academic Press, New York (1997)).

Several web sites and database contain information pertaining to biochemical pathways and regulatory pathways. Examples of such web sites or data bases include: http://cgsc.biology.yale.edu (the CGSC maintains a database of E. coli genetic information, including genotypes and reference information for the strains in the CGSC collection, gene names, properties, and linkage map, gene product information, and information on specific mutations); http://www.labmed.umn.edu (the University of Minnesota's Biocatalysis/Biodegradation web page provides a search engine for compounds, enzymes, microorganisms, chemical formulas CAS registry, EC accession and microbial biocatalytic reactions and biodegradation pathways primarily for xenobiotic, chemical compounds such methionine, and threonine); http://wit.mcs.anl.gov/WIT2 (this website provides a functional overview which outlines metabolic pathways for organisms such as E. coli); http://ecocyc.PangeaSystems.com/ecocvc/ecocvc.html (this web site provides an overview of an E. coli metabolic map); http://www.biology.UCSD.edu (this web site provides information on signal transduction in higher plants); http://geo.nihs.go.jp (the Japanese National Institute of Health Science server provides information particularly on cell signaling networks); http://gifts.univ-mrs.fr (the Gene Interactions in Fly Trans-world Server provides information on gene interactions, mostly centered on Drosophila gene interactions); http://sdb.bio.purdue.edu (this web site provides a data base of Drosophila genes); http://genome-www.stanford.edu (Stanford Genomic Research web site provides information on for example, Sacchromyces and Arabidopsis); http://www.psynix.co.uk (this web site provides illustrations and computer models of various cytokinins); http://www.sdsc.edu/Kinases/pk home.html (this web site provides information on the protein kinase family of enzymes); http://transfac.gbf-braunschweig.de (the GBF web site provides information on regulatory genomic signals and regions, in particular those that govern transcriptional control); http://www.gcrdb.uthscsa.edu (this web site provides information on G-protein coupled receptors); http://www.biochem.purdue.edu (this web site provides information on secondary metabolism in Arabidopsis); http://home.wxs.nl/˜pvsanten/mmp/mmp.html (this web site provides a flow chart of metabolic pathways); http://www.genome.ad.jp/kegg/regulation.html (this web site, the KEGG regulatory pathways web site, provides pathway maps, ortholog group tables, and molecular catalogs searchable data bases by enzyme, pathway, or EC number); http://capsulapedia.uchicago.edu/Capsulapedia/Metabolism/RepExpMet.shtml (this web site provides expression information); http://www.zmbh.uni-heidelberg.de/M_pneumoniae/genome/META/ALL_META.GIF (this web site provides a graphic of metabolic pathways and the ways these pathways interact); http://moulon.inra.fr/cgi-bin/nph-acedb3.1/acedb/metabolisme (this web site provides information on C. elegans metabolic enzymes); http://www.gwu.edu/˜mpb (this web site provides information on metabolic pathways); http://www.bic.nus.edu.sg/pathwaydb.html (this web site provides links to biological pathways, such as metabolic pathways, developmental pathways, signal-transduction pathways, and genetic regulatory circuits); and http://www.scri.sari.ac.uk/bpp/charttxt.htm (this web site provides graphics of the metabolic pathways of diseased potato).

Illustrative pathways are set forth in more detail below.

A. Photosynthesis

1. Biosynthesis of Tetrapyrroles

The biosynthesis of tetrapyrroles such as heme and chlorophyll as well as a number of other tetrapyrroles such as siroheme, the cofactor for sulfite and nitrite reductases, cobalamin (vitamin B12), and the chromophore of phytochrome, can be subdivided into three major phases; ALA synthesis, porphyrin ring synthesis and synthesis of final products. The pathway is conserved among species except for the synthesis of 5-aminolevulinate, also known as 5-aminolevulinic acid (“ALA”) (Porra, Photochemistry and Photobiology 65:492-516 (1997); von Wettstein et al., Plant Cell 7:1039-1057 (1995)).

The first phase of the biosynthesis of tetrapyrroles, such as heme and chlorophyll, is the synthesis of ALA. Yeast, fungi, mammals and some bacteria (the α-group of proteobacteria or purple bacteria, e.g. Bradyrhizobium japonicum and Rhodobacter capsulatus) biosynthesize tetrapyrroles via the single step four-carbon (C4), or Shemin pathway. In this pathway ALA synthase (E.C. 2.3.1.37) catalyzes the condensation of glycine with succinyl-CoA to generate ALA.

Plants, green algae, cyanobacteria, most eubacteria (e.g., E. coli and Bacillus subtilis), and archaebacteria biosynthesize ALA via the three-step five-carbon (“C5”) pathway, which includes glutamyl-tRNA synthetase (“GluRS”), glutamyl-tRNA reductase (“GluTR”) and glutamate-1-semialdehyde aminotransferase (“GSA-AT”). In plants and algae, the C5 pathway is localized in the chloroplast. The formation of ALA via the C5 pathway is reported to be the rate-limiting step in the biosynthesis of heme and chlorophyll (Kumar et al., Trends in Plant Science 1:371-376 (1996); Tanaka et al., Plant Physiol. 110:1223-30 (1996); Masuda et al., Plant Physiol. Biochem. 34:11-16 (1996); Hungerer et al., J. Bacteriol. 177:1435-43 (1995); Ilag et al., Plant Cell 6:265-75 (1994)).

Chloroplastic GluRS (E.C. 6.1.1.17), also known as glutamate-tRNA ligase, converts glutamate to glutamyl-tRNA (“Glu-tRNA”) activating the C-1 of glutamate in an ATP dependent reaction (Porra, Photochemistry and Photobiology 65:492-516 (1997); von Wettstein et al., Plant Cell 7:1039-1057 (1995)). Glu-tRNA is reported to be the first intermediate in the C5 pathway and it also reported to serve as a source of glutamate in protein biosynthesis. GluRS is a soluble plastid enzyme which has been isolated from higher plants (barley, wheat) and other organisms. Reported GluRS enzymes are homodimers encoded by a nuclear gene and synthesized in the cytoplasm and have a molecular weight of 54 kD (barley) and 56 kD (wheat).

GluTR, the first committed enzyme reported in heme and chlorophyll biosynthesis, catalyzes the NADPH dependent reduction of Glu-tRNA to glutamate 1-semialdehyde (“GSA”) with the release of intact tRNA (Porra, Photochemistry and Photobiology 65:492-516 (1997); von Wettstein et al., Plant Cell 7:1039-1057 (1995)). GluTR is reported as the rate limiting step in ALA formation and is present only at low levels in all organisms examined (Masuda et al., Plant Physiol. Biochem. 34:11-16 (1996); Schroeder et al., Biochem. J. 281:843-50 (1992); Masuda et al., Plant Cell Physiol. 36:1237-43 (1995)). Plant GluTR is a soluble enzyme localized in plastids and encoded in the nucleus. GluTR has been reported to exist as a multimer of a single subunit. The purified barley enzyme has a molecular weight of 270 kD with a monomeric subunit size of 54 kD (Pontoppidan and Kannangara, Eur. J. Biochem. 225:529-37 (1994)). Arabidopsis and cucumber enzymes have similar subunit molecular weights (Tanaka et al., Plant Physiol. 110:1223-30 (1996); Ilag et al., Plant Cell 6:265-75 (1994); Kumar et al., Plant Mol. Biol. 30:419-26 (1996)).

GluTR genes (also known as HEMA genes) have been cloned and the amino acid sequences determined for a number of sources including three higher plants; Arabidopsis, barley, and cucumber. The deduced amino acid sequence of GluTR from all sources exhibit about 60% overall similarity with stretches of amino acid identity. Barley, Arabidopsis, and cucumber show over 70% identity at the deduced amino acid level (Vothknecht et al., Proc. Natl. Acad. Sci. (U.S.A.) 93:9287-9291 (1996)). Two different GluTR genes have been isolated from three higher plants; Arabidopsis (Ilag et al., Plant Cell 6:265-75 (1994)), barley (Bougri and Grimm, Plant J. 9:867-878 (1996)), and cucumber (Masuda et al., Plant Cell Physiol. 36:1237-43 (1995)). In Arabidopsis and cucumber, one GluTR gene is expressed in all tissues and a second is expressed in a tissue specific manner. These genes are also reported to be differentially regulated by light (Tanaka et al., Plant Physiol. 110:1223-30 (1996); Masuda et al., Plant Physiol. Biochem. 34:11-16 (1996); Ilag et al., Plant Cell 6:265-75 (1994); Masuda et al., Plant Cell Physiol. 36:1237-43 (1995); Kumar et al., Plant Mol. Biol. 30:419-26 (1996); Hori et al., Plant Physiol. Biochem. 34:3-9 (1996)).

GSA-AT (glutamate-1-semialdehyde aminotransferase (E.C. 5.4.3.8)), catalyzes the conversion of GSA to ALA. GSA-AT is a soluble protein localized in the chloroplast and encoded in the nucleus (Porra, Photochemistry and Photobiology 65:492-516 (1997); von Wettstein et al., Plant Cell 7:1039-1057 (1995)). It has a subunit molecular weight of about 45 kD. The holoenzyme consists of two identical subunits and utilizes pyridoxal phosphate (“PLP”) as a cofactor (Kumar et al., Trends in Plant Science 1:371-376 (1996); Gough et al., Glutamate 1-semialdehyde aminotransferase as a target for herbicides, Boeger, Ed., Lewis, Boca Raton, Fla., (1993)). GSA-AT is reported to be inhibited by gabaculine, which has also been shown to inhibit chlorophyll biosynthesis in barley leaves (Rogers and Smith, BCPC Monogr. 42:183-93 (1989)). GSA-AT has been crystallized from Synechococcus (Hennig et al., J. Mol. Biol. 242:591-594 (1994); Hennig et al., Proc. Natl. Acad. Sci. (U.S.A.) 94:4866-4871 (1997)).

GSA-AT genes have been cloned from a number of plants including Arabidopsis. The deduced amino acid sequences from plants are highly conserved. As with GluTR, two GSA-AT genes have been found in Arabidopsis and they may be differentially regulated by light. It has been reported that the presence of two genes for both enzymes of the C5 pathway indicate that there are two routes for ALA formation in chloroplasts (Kumar et al., Trends in Plant Science 1:371-376 (1996)). Transgenic tobacco plants that express antisense RNA to GSA-AT have been reported to show varying degrees of chlorophyll deficiency. Antisense plants with chlorophyll contents less than about 25% of that in the wild type plants which were maintained in the greenhouse under high light conditions, did not survive (Hennig et al., Proc. Natl. Acad. Sci. (U.S.A.) 94:4866-4871 (1997); Hoefgen, et al., Proc. Natl. Acad. Sci. (U.S.A.) 91:1726-1730 (1994)).

The second phase of the biosynthesis of tetrapyrroles involves the formation of the porphyrin ring. The intermediates involved in this portion of the chlorophyll/heme biosynthetic pathway, from ALA to protoporphyrin IX, appear to be essentially the same in all organisms including plants and mammals.

Porphobilinogen synthase (E.C. 4.2.1.24), also known as ALA dehydratase, catalyzes the asymmetric condensation of two molecules of ALA to yield porphobilinogen (Porra, Photochemistry and Photobiology 65:492-516 (1997); von Wettstein et al., Plant Cell 7:1039-1057 (1995)). Porphobilinogen synthase is a metalloenzyme and there are different types of the enzyme categorized according to metal ion usage. Porphobilinogen synthase has been identified in several plants including spinach, pea, tomato, radish, and soybean. In higher plants the enzyme is located in the plastid, is a hexamer (40-50 kD subunits) and binds Mg⁺². The mammalian enzyme is an octamer and binds Zn²⁺ (Cheung et al., Biochemistry 36:1148-1156 (1997); Senior et al., Biochem. J. 320:401-412 (1996)). Several studies have shown that porphobilinogen synthase is both developmentally and light regulated in plants (Kyriacou et al., J. Am. Soc. Hortic. Sci. 121:91-95 (1996)).

Hydroxymethylbilane synthase (E.C. 4.3.1.8), also known as porphobilinogen deaminase, catalyzes the formation of the linear tetrapyrrole hydroxymethylbilane (Porra, Photochemistry and Photobiology 65:492-516 (1997); von Wettstein et al., Plant Cell 7:1039-1057 (1995)). The reaction involves the deamination and polymerization of four molecules of the monopyrrole porphobilinogen. Hydroxymethylbilane synthase is unusual in that it contains a novel dipyrromethane cofactor at the active site, which is self-assembled by the apoenzyme and is covalently attached to an invariant cysteine. The enzyme has been identified in mammals, yeast, bacteria, and plants (e.g., pea, spinach, Arabidopsis). Hydroxymethylbilane synthase exists as a monomer with a molecular weight of 33-44 kD. Hydroxymethylbilane synthase from Arabidopsis has been cloned and found to be localized in the plastid in both roots and leaves (Witty et al., Planta 199:557-564 (1996)). The 3-dimensional structure of porphobilinogen deaminase from E. coli has been determined (Louie et al., Proteins: Struct., Funct., Genet. 25:48-78 (1996)).

Uroporphyrinogen III (co)synthase (E.C. 4.2.1.75) catalyzes the ring closure of the unstable linear tetrapyrrole hydroxymethylbilane and the simultaneous isomerization of the acetyl and propionyl groups at pyrrole ring D forming uroporphyrinogen III (Porra, Photochemistry and Photobiology 65:492-516 (1997); von Wettstein et al., Plant Cell 7:1039-1057 (1995)). Uroporphyrinogen III (co)synthase has been isolated from a number of sources including mammals, bacteria, and plants (spinach). Uroporphyrinogen III (co)synthase has a molecular weight of about 30 kD and is highly diverse in primary structure depending on the source.

Uroporphyrinogen III decarboxylase (E.C. 4.1.1.37) catalyzes the stepwise decarboxylation of all four acetate side chains of uroporphyrinogen III starting with ring D followed by rings A, B, and C, respectively, to form coproporphyrinogen III (Porra, Photochemistry and Photobiology 65:492-516 (1997); von Wettstein et al., Plant Cell 7:1039-1057 (1995)). At high substrate concentrations, decarboxylation can occur randomly. Uroporphyrinogen III decarboxylase has been isolated from mammals, yeast, bacteria and plants (e.g., tobacco, barley). It is a monomeric enzyme with a molecular weight of about 40 kD. The barley and tobacco enzymes are reported to be light regulated (Mock et al., Plant Mol. Biol. 28:245-256 (1995)). Antisense tobacco plants have been generated and decreased levels of the enzyme were accompanied by a light-dependent necrotic phenotype and accumulation of uroporphyrinogen. It has been reported that the lesions may be caused by reactive oxygen species generated by photooxidized uroporphyrinogen (Mock et al., Plant Mol. Biol. 28:245-256 (1995)).

In aerobic organisms including plants, coproporphyrinogen III oxidase (E.C. 1.3.3.3), catalyzes the oxygen dependent sequential oxidative decarboxylation of the A and B propionyl side chains of coproporphyrinogen III to yield two vinyl groups and protoporphyrinogen IX (Porra, Photochemistry and Photobiology 65:492-516 (1997); von Wettstein et al., Plant Cell 7:1039-1057 (1995)). A separate enzyme is reported to catalyze the anaerobic reaction.

Coproporphyrinogen III oxidase has been studied in a number of organisms including plants (tobacco, pea). The enzyme is a homodimer and has a subunit molecular weight of about 35-40 kD and is located in plastids. It has been reported that coproporphyrinogen III oxidase is peripherally associated with the membrane. It has been isolated from soybean, barley and tobacco and these sequences show 70% identity at the amino acid level. Transcript levels are reportedly similar in etiolated and green leaves (barley) but higher in developing cells than in mature cells (Kruse et al., Planta 196:796-803 (1995)). Antisense tobacco plants have been reported with decreased levels of the enzyme. The decreased level was accompanied by accumulation of coproporphyrinogen, slightly reduced chlorophyll content and a necrotic phenotype. The prominent phenotype indicates photodynamic damage (Kruse et al., EMBO J. 14:3712-3720 (1995)).

Protoporphyrinogen IX oxidase (E.C. 1.3.3.4) catalyzes the formation of the aromatic protoporphyrin IX by the six electron oxidation of protoporphyrinogen IX (Porra, Photochemistry and Photobiology 65:492-516 (1997); von Wettstein et al., Plant Cell 7:1039-1057 (1995)). This is the last reported common step in tetrapyrrole biosynthesis. In aerobic organisms, the reaction is catalyzed by a flavoprotein that utilizes oxygen as an oxidant and, under anaerobic conditions, the oxidation is achieved by passing electrons to the electron transport chain. The enzyme has been purified from a number of sources including mammals and plants (barley) and is an integral membrane protein. The barley enzyme has a molecular weight of 36 kD and activity has been found in both plastidal and mitochondrial extracts.

The plastidal and mitochondrial forms of protoporphyrinogen IX oxidase have been cloned from tobacco and were found to exhibit low homology. The mitochondrial form is associated with heme biosynthesis. The plastidic enzyme functions primarily in the formation of chlorophyll and to a lesser extent in the formation of heme required for plastid proteins (Lermontova et al., Proc. Natl. Acad. Sci. (U.S.A.) 94:8895-8900 (1997)). Protoporphyrinogen IX oxidase is susceptible to inhibition by a number of herbicides including diphenyl ethers. Phytotoxicity has been explained as due to the accumulation of excess protoporphyrinogen which is rapidly oxidized to protoporphyrin in the cytoplasm. Protoporhyrin has been reported as a potent photosensitizer which generates singlet oxygen and causes rapid lipid peroxidation and cell death.

In the third and final phase of tetrapyrrole biosynthesis, magnesium or iron is inserted into protoporphyrin IX and subsequent modifications lead to the synthesis of the final tetrapyrrole products, such as chlorophyll and heme.

Mg-chelatase catalyzes the conversion of protoporphyrin IX to magnesium protoporphyrin IX by the insertion of Mg⁺² (Porra, Photochemistry and Photobiology 65:492-516 (1997); von Wettstein et al., Plant Cell 7:1039-1057 (1995)). Mg-chelatase, which requires ATP, is reportedly a three component enzyme. The three protein components have molecular weights of about 140, 40, and 70 kD. The reaction takes place in two steps, an ATP-dependent activation followed by an ATP-dependent chelation step. Mg-chelatase activity has been demonstrated in peas, cucumber, and barley and reportedly is localized in the chloroplast. Barley, Arabidopsis, and soybean genes encoding the 140 and 40 kD subunits have been cloned. Studies with the two identified plant genes show that Mg-chelatase expression is light regulated (Walker and Willows, Biochem. J. 327:321-333 (1997)).

Mg-protoporphyrin IX O-methyltransferase (E.C. 2.1.1.11) esterifies the propionic side chain of ring III of Mg-protoporphyrin IX to form Mg-protoporphyrin IX monomethylester (Porra, Photochemistry and Photobiology 65:492-516 (1997); von Wettstein et al., Plant Cell 7:1039-1057 (1995)). The methyl group is donated by the cofactor S-adenosyl-L-methionine. The enzyme has been isolated from bacteria and plants (wheat). A gene for Mg-protoporphyrin IX O-methyltransferase has been cloned from bacteria including Synechocystis (Smith et al., Plant Mol. Biol. 30:1307-1314 (1996)).

Mg-protoporphyrin IX monomethyl ester cyclase catalyzes the cyclization of Mg-protoporphyrin IX monomethylester to form the isocyclic ring E of divinyl protochlorophyllide (Porra, Photochemistry and Photobiology 65:492-516 (1997)). In aerobic organisms the enzymatic reaction is dependent on O₂ and NADPH. Evidence suggests that Mg-protoporphyrin IX monomethyl ester cyclase is a membrane-bound monooxygenase of the iron-sulfur protein or copper protein type. Mg-protoporphyrin IX monomethyl ester cyclase has been extracted from chloroplasts of higher plants including cucumber and wheat. A cucumber enzyme has been shown to consist of two components, a soluble and a membrane-bound component. The soluble component has a molecular weight of 30 kD (Bollivar and Beale, Plant Physiol. 112:105-114 (1996)).

The reduction of divinyl protochlorophyllide to monovinyl protochlorophyllide has been reported based on product characterization, this reaction is catalyzed by 8-vinyl reductase (Porra, Photochemistry and Photobiology 65:492-516 (1997)). It has been reported that Mg-protoporphyrin IX monomethylester may also act as a substrate. NADPH is the most likely reductant. 8-vinyl reductase has been detected in higher plants including wheat and cucumber.

Protochlorophyllide reductase (“POR”) (E.C. 1.3.1.33) catalyzes the reduction of the double bond between carbons 7 and 8 of the D ring of protochlorophyllide producing chlorophyllide (Porra, Photochemistry and Photobiology 65:492-516 (1997); von Wettstein et al., Plant Cell 7:1039-1057 (1995)). In angiosperms this is a light-dependent reaction. Non-flowering land plants, algae, and cyanobacteria contain both a light-dependent and a light-independent enzyme. Some other organisms contain only the light-independent enzyme. Three chloroplast genes have been identified that are essential for the light-independent enzyme (chlL, chlN and chlB).

The light-dependent POR (“L-POR”) has been purified from barley, oat, and Arabidopsis. L-POR has a molecular weight of 35-38 kD and forms different multimers and aggregates with other proteins. L-POR is localized in the plastid and encoded in the nucleus. Genes encoding L-POR have been cloned from, for example, barley, Arabidopsis, pea, and oat. Two distinct and differentially light-regulated L-POR genes, POR A and POR B, have been identified in Arabidopsis and barley. POR A and POR B have biochemically equivalent light-dependent activities, with different expression patterns. POR B is reported to be present throughout the plant life cycle, while POR A is reported to function only in the very early stages of greening of etiolated tissue (Runge et al., Plant J. 9:513-523 (1996); Holtorf and Apel, Plant Mol. Biol. 31:387-392 (1996); Martin et al., Biochem. J. 325:139-145 (1997)).

Chlorophyll synthetase catalyzes the last reported step in chlorophyll a biosynthesis (Porra, Photochemistry and Photobiology 65:492-516 (1997); von Wettstein et al., Plant Cell 7:1039-1057 (1995)). Chlorophyll synthetase esterifies the propionic acid side chain of ring D of chlorophyllide with either phytyl pyrophosphate in green plants or geranylgeranyl pyrophosphate in greening etiolated seedlings. The enzyme is located in the plastid. A gene that encodes the enzyme in Synechocystis (chlG) and a gene that encodes the enzyme in Arabidopsis (G4) have been cloned and expressed in E. coli. The Synechocystis enzyme has the preferred substrate specificity reported for green plants. The cloned and expressed enzyme from Arabidopsis has the preferred substrate specificity reported for etiolated plants (Oster et al., J. Biol. Chem. 272:9671-9676 (1997); Oster and Rudiger, Bot. Acta 110:420-423 (1997).

Ferrochelatase (E.C. 4.99.1.1) catalyzes the conversion of protoporphyrin IX to heme. In plants the enzyme is located in both mitochondria and plastids. Ferrochelatase is reported to be a single soluble protein. Two ferrochelatase genes have been identified in Arabidopsis. Ferrochelatase-II encodes a protein targeted to the chloroplast and ferrochelatase-I encodes a protein targeted to both chloroplasts and mitochondria (Roper and Smith, Eur. J. Biochem. 246:32-37 (1997); Chow et al., J. Biol. Chem. 272:27565-27571 (1997)).

2. Phytochrome Protein

Light is essential for normal plant growth and development not only as a source of energy but also as an environmental signal regulating various developmental, physiological and metabolic processes. Light-regulated responses occur throughout the entire life cycle of a plant, including seed germination, seedling de-etiolation, leaf development, chloroplast biogenesis and development, flowering, senescence, effective utilization of carbon between vegetative and reproductive tissues, and responses to environmental factors (Kendrick and Kronenberg, In: Photomorphogenesis in Plants. Martinus Nijhoff, Dordrecht eds. (1994)). Perception and transduction of the light signals have been reported to be governed by at least three families of receptors, including the phytochromes (red and far-red) receptors, blue-light receptors, and UV receptors (Deng, Cell 76:423-426 (1994); Quail et al., Science 268:675-680 (1995)). In addition to light-regulated development and gene expression, it has been reported that some light-inducible genes are also regulated by circadian rhythm (Piechulla, Plant Mol. Biol. 22:533-542 (1993); Taylor, Plant Cell 1:259-264 (1989); Guiliano et al., EMBO Journal 7:3635-3642 (1988)).

Phytochrome is a light-sensing protein-chromophore complex present in higher plants. At least five species of phytochrome, designated phyA to phyE, have been reported in Arabidopsis thaliana (Sharrock and Quail, Genes Dev. 3:1745-1757 (1989); Clack et al., Plant Mol. Biol. 25:413-427 (1994)) and it has been reported that some of these photosensory phytochromes have both overlapping and unique functions in plants (Reed et al., Plant Physiol. 104:1139-1149 (1994); Smith, Annu. Rev. Plant Physiol. Plant Mol. Biol. 46:289-315 (1995)). Phytochromes have been reported to be associated with the establishment of a plant's circadian clock and its floral initiation rate (Anderson and Kay, Trends in Plant Sciences 1:51-57 (1996); Weller et al., Plant Physiol. 114:1225-1236 (1997); Weller et al., Trends in Plant Sciences 2:412-418 (1997)). Several physiological modes of light regulations associated with phytochromes, including very low fluence response, low fluence response, high radiance response, end-of-day far-red response and the shade avoidance to red:far-red ratio, have been reported (McCormac et al., Plant Journal 4:19-27 (1993); Smith and Whitelam, Plant Cell Environ. 13:695-707 (1990)).

Reported phytochrome apoproteins are between 120 and 130 kilodaltons in size, and are found in the cytoplasm as dimers. Each monomer has been reported to fold into two major structural domains separated by a protease-sensitive hinge region. Reported phytochrome molecules also have two functional domains. An approximately 70 kilodalton amino terminal domain has been reported to be associated with photosensory specificity of phytochrome. The portion of the molecule necessary for dimerization and signal transduction has been reported to be associated with an approximately 55 kilodalton carboxy terminal end. In several members of the phytochrome family, it has been reported that the initial response to light occurs through a chromophore covalently bound to the polypeptide chain. For all five reported members of the Arabidopsis phytochrome family, the chromophore has been reported to be a linear tetrapyrrole, responsible for the absorption of visible light. Phytochromes have been reported to undergo a light-induced reversible interconversion between two molecular isoforms, known as the Pᵣ and the P_{fr} forms. The Pᵣ form absorbs red photons (λₘₐₓ, 665 nm) to assume the conformation known as the P_{fr} form. It is this “activation” which has been reported as a signal for the cell to respond. Upon exposure to far-red photons (λₘₐₓ, 730 nm), this particular molecule has been reported to respond in one of two ways. The P_{fr} form may return to the conformation of the Pᵣ form or the protein may be rapidly degraded. It is the interconversion of the Pᵣ and P_{fr} forms which has been reported to operate as a trigger for growth and developmental responses by altering gene expression in the cell.

It has been reported in a number of eukaryotic and prokaryotic organisms that phytochromes may be protein kinases. Autophosphorylation of purified phytochrome proteins and sequence homology between the photoreceptors and eukaryotic protein kinases have been reported (Wong et al., Plant Physiol. 91:709-718 (1989); Thummler et al., FEBS Lett. 357:149-155 (1995); Quail, BioEssays 19:571-579 (1997); Elich and Choury, Cell 91:713-716 (1997)). Phytochromes have been reported to lack the consensus sequences that define protein kinases in eukaryotes. The C-terminal 250 amino acid sequence of phytochrome C has been reported to have similarity to a transmitter histidine protein kinases of the two component systems of prokaryotes (Schneider-Poetsch, Photochem. and Photobiol. 56:839-846 (1992)). Two reported gene sensory proteins of blue-green algae are related to higher plant phytochromes by amino acid homologies in the N-terminal regions. Both have also been reported to have homology to histidine kinases (Quail, Plant, Cell and Environment 20:657-665 (1997); Kehoe et al., Science 273:1409-1412 (1996)). The C-terminus of phytochrome protein has been reported to contain a domain adjacent to a hinge region with reported homology energy-sensing proteins, including histidine kinases (Yeh et al., Science 277:1505-1508 (1997); Zhulin et al., Trends Biochem. Sci. 22:331-333 (1997)). Phytochromes have been reported to function in transduction of light signals through kinase activity that activates one or more G proteins to induce or to shut off transcription of nuclear genes in the specific cells in which the phytochromes are expressed. Some of the genes whose light-regulated expression has been reported to be mediated by phytochromes include regulatory proteins such as nitrate reductase, chlorophyll a/b binding protein, catalase, RUBISCO small subunit protein, and photosystem II proteins (Chandok and Sopory, Mol. Gen. Genet. 251:599-608 (1996); Anderson and Kay, Adv. in Genetics, Vol. 34 Academic Press (1994)).

Protein sequences similar to plant phytochromes have been reported from Synechocystis strain PCC6803, and Fremyella diplosiphon, two prokaryotic algae (Quail, Plant, Cell and Environment 20:657-665 (1997); Kehoe et al., Science 273:1409-1412 (1996)). In the case of Fremyella diplosiphon, the protein is reported to be a chromatic adaptation sensor.

The number of phytochrome genes is not reported to be uniform among all plants. Five members of the phytochrome family in Arabidopsis, named A, B, C, D, and E, which are similar in nucleic acid and protein sequences, functions and wavelength to which they respond, have been reported (Sharrock and Quail, Genes Dev. 3:1745-1757 (1989); Clack et al., Plant Mol. Biol. 25:413-427 (1994); Pratt, Photochem. Photobiol. 61:10-21 (1995)). Amino acid homologies between these five Arabidopsis proteins have been reported to be between 46 and 80% with the greatest dissimilarities being at the amino and carboxy-termini. Functional analysis of phytochromes has been reported using photomorphogenic mutants lacking a particular phytochrome. Such mutations are reported to have pleotropic effects on plant development. Constitutive expression of phytochromes have been reported, including constitutive expression of phytochrome A (phyA).

Phytochrome A has also been reported to be associated with early seedling establishment and survival. In a number of plants, phytochromes, such as phy A, have been reported to mediate far-red high irradiance responses such as response of seeds to light environmental cues. Phytochrome A has been reported to accumulate to high levels in etiolated seedlings, in which it mediates the inhibition of stem growth. In response to far-red and red light, it has been reported that phy A promotes seed germination and seedling de-etiolation (increases chlorophyll biosynthesis to result in green color and stem growth) and plays a crucial role in flowering. Phy A has been reported to control flowering in pea by reducing the level of an inhibitor to flower formation (Weller et al., Trends in Plant Sciences 2:412-418 (1997); Botto et al., Plant Physiol. 110:439-444 (1996)). Phy A has been reported to be the only member of the Arabidopsis gene family that predominates in etiolated plant tissues. Activation to the P_{fr} conformer results in a rapid turnover.

Phytochrome B is a subfamily, which contains two reported members in Arabidopsis, B1 and B2. These members of the phytochrome B subfamily have been reported to be associated with the red/far-red reversible response. Phy B mutants (phyB-) have been reported to exhibit an early-flowering phenotype (Weller et al., Planta 189:15-23 (1993); Coupland, Trends Genet. 11:393-397 (1995)). Phytochromes A and B have been reported to have reciprocal sensitivities. Phytochrome B has been reported to be associated with a shade avoidance role later in development. Overexpression of an oat phy A gene in tobacco and Arabidopsis has been reported to disable shade avoidance responses to red:far-red ratio (Robson et al., Nature Biotechnology 14:995-998 (1996)). Shade avoidance has been reported to play a role for the light-stable phytochrome pool, including phy B. Reported mutants lacking a B type phytochrome include the long hypocotyl mutant (1 h) of cucumber, the elongated internode mutant (ein) in Brassica napus, the tomato tri mutant and at least one of the maturity mutants (ma₃^{R}) of Sorghum bicolor (Lopez-Juez et al., Plant Cell 4:241-51 (1992); Devlin et al., Plant Physiol. 100: 1442-47 (1992); Reed et al., Plant Cell 5:137-147 (1993); Foster et al., Plant Physiol. 105:941-48 (1994); Childs et al., Plant Physiol. 113:611-619 (1997)). Phy B and the photoreceptors of Arabidopsis have been reported to predominate in extracts of green plants. The P_{fr} form has been not reported to be degraded but is slowly reconformed to the Pᵣ structure.

Phytochrome C protein of Arabidopsis has been reported to have a photosensory specificity similar to phy B and have a role in primary leaf expansion (Qin et al., Plant J. 12:1163-1172 (1997). It has been reported that the expression of heterologous phytochromes A, B or C in transgenic tobacco plants altered vegetative development and flowering time (Halliday et al., Plant J. 12:1079-1090 (1997)).

Phy D has been reported to be related to phy B by nucleic acid homology. A reported deletion mutant of the phy D gene in Arabidopsis has been reported to resemble the mutants which lacked phy B (Aukerman et al., Plant Cell 9:1317-1326 (1997).

Several dicots have been reported to have additional phytochrome or phytochrome-like genes. Expression of phytochrome A genes has been evaluated in Fabaceace, Solanaceae, and Caryophyllaceae (Adam et al., Plant Physiol. 101:1407-1408 (1993); Matthews et al., Ann. Missouri Bot. Gard. 82:296-321 (1995)). Plants which contain additional phytochrome or phytochrome-like genes have been reported to belong to at least three plant families, the Cruciferae, Solanaceae and Umbelliferae. In tomato, with 7 reported genes, two phytochrome-like proteins are reported to mediate a phy B-type response (Pratt et al., Planta 197:203-206 (1995).

3. Carbon Assimilation Pathway

The primary sites of photosynthetic activity, generally referred to as “source organs”, are mature leaves and to a lesser extent, other green tissues (e.g., stems). Photosynthesis may be broadly divided into two phases: a light phase, in which the electromagnetic energy of sunlight is trapped and converted into ATP and NADPH, and a dark or synthetic phase, in which the ATP and NADPH generated by the light phase are used, in part, for biosynthetic carbon reduction. In most plants, the major products of photosynthesis are starch (transitory storage form of carbohydrate formed in chloroplasts), and sucrose (formed in the cytosol). Sucrose represents the predominant form of carbon transport in higher plants. Processes that play a role in plant growth and development, crop yield potential and stability, and crop quality and composition include: enhanced carbon assimilation, efficient carbon storage, and increased carbon export and partitioning.

Oxygen-evolving organisms are reported to have a common pathway for the reduction of CO₂ to sugar phosphates. This pathway is known as the reductive pentose phosphate (RPP), Calvin-Benson or C3 cycle (Calvin and Bassham, The Photosynthesis of Carbon Compounds, Benjamin, N.Y. (1962); Bassham and Buchanan, In: Photosynthesis, Govindjee, ed., Academic Press, New York, 141-189 (1982). A number of plants exhibit adaptations in which CO₂ is first fixed by a supplementary pathway and then released in cells in which the RPP cycle operates. From the point of view of the metabolic pathway operating for photosynthetic carbon assimilation, higher plants can be classified by the existence of supplemental pathway such as C3, C4, and crassulacean acid metabolism species (Edwards and Walker, C3-C4: Mechanism and cellular and environmental regulation of photosynthesis, Blackwell Scientific Publications, Oxford, (1983)).

The RPP pathway is reported to be the main route by which CO₂ is ultimately incorporated into organic compounds in all species of higher plants (Edwards and Walker, C3-C4: Mechanism and cellular and environmental regulation of photosynthesis, Blackwell Scientific Publications, Oxford, (1983); Macdonald and Buchanan, In: Plant Physiology, Biochemistry and Molecular Biology, Dennis and Turpin (eds.), J. Wiley & Sons, Inc., New York, p. 239 (1990); Robinson and Walker, In: The Biochemistry of Plants, Vol. 8, Hatch and Boardman (eds.), Academic Press, New York, p. 193 (1981)). In C3 plants, the RPP pathway is the sole route for photosynthetic carbon assimilation, whereas in C4 and CAM plants an additional (not alternative) method of carbon fixation, is present separated in space (C4 plants) or in time (CAM plants) from the RPP cycle (Edwards and Walker, C3-C4: Mechanism and cellular and environmental regulation of photosynthesis, Blackwell Scientific Publications, Oxford, (1983)). Carbon skeletons are required to incorporate other functional groups, the operation of the RPP cycle for photosynthetic CO₂ fixation is a requisite for the biochemical synthesis of carbohydrates, lipids, proteins, and nucleic acids.

i. The Reductive Pentose Phosphate Cycle

The RPP cycle is reported to be the primary carboxylating mechanism in plants. Enzymes which catalyze steps in the RPP cycle are water soluble and are located in the soluble portion of the chloroplast (stroma). Reviews on the mechanism and enzymes involved in the RPP cycle include: Bhagwat, In: Handbook of Photosynthesis, Pessaraki, ed., Marcel Dekker Inc, New York, 461-480 (1997); Iglesias et al., In: Handbook of Photosynthesis, Pessaraki, ed., Marcel Dekker Inc, New York, 481-503 (1997); Robinson and Walker, In: The Biochemistry of Plants, Vol. 8, Hatch and Boardman, eds., Academic Press, New York, 193-236 (1981); Macdonald and Buchanan, In: Plant Metabolism, Dennis et al., eds., Longman, Essex, England, 299-313 (1997).

The RPP pathway is an autocatalytic pathway for the de novo synthesis of carbohydrates from inorganic CO₂. The RPP cycle is reported to comprise three phases. The first phase of the cycle is the carboxylation phase, during which ribulose-1,5-biphosphate (Rbu-1,5-P₂) is carboxylated to produce two molecules of 3-phosphoglycerate (3-PGA). The next phase is the reductive phase during which ATP and NADPH produced by the light reaction of photosynthesis are consumed in the reduction of 3-PGA to glyceraldehyde-3-phosphate (GA-3-P). The RPP cycle is completed by the regeneration phase where intermediates formed from GA-3-P are utilized via a series of isomerizations, condensations and rearrangements, resulting in the conversion of five molecules of triose phosphate to three molecules of pentose phosphate, and eventually ribulose 5-phosphate (Rbu-5-P). Phosphorylation of Rbu-5-P by ATP regenerates the original carbon acceptor Rbu-1,5-P₂, thus completing the cycle.

The RPP cycle is a metabolic pathway common to all photosynthetic organisms. Many of the enzymes of the metabolic route, as well as proteins involved in metabolite transport and regulation, have been purified.

Ribulose biphosphate carboxylase (RUBISCO, also referred to as ribulose-1,5-biphosphate carboxylase/oxygenase (EC 4.1.1.39)) constitutes about 50% of the total soluble protein in green leaves. Ribulose biphosphate carboxylase is reported to provide a quantitative link between the pools of inorganic and organic carbon in the biosphere. Ribulose biphosphate carboxylase catalyses the conversion of atmospheric carbon dioxide into three carbon compounds. Subsequent reactions result in both regeneration of the acceptor molecule and translocation of three molecules of triose-phosphate to the cytosol for synthesis of sucrose and starch. Reviews of the ribulose biphosphate carboxylase enzyme are provided by Ellis, Trends Biochem. Sci. 4:241-244 (1979); Hartman and Harpel, Annu. Rev. Biochem. 63:197-234 (1994); Miziorko and Lorimer, Annu. Rev. Biochem. 52:507-535 (1983); Andrews and Lorimer, In: The Biochemistry of Plants, Vol. 10, Hatch and Boardman, eds., Academic Press, San Diego, p. 131 (1987); Jensen, In: Plant Physiology, Biochemistry, and Molecular Biology, Dennis and Turpin, eds., J. Wiley & Sons, Inc., New York, p. 224 (1990).

Plants are reported to have two phosphoglycerate kinase isoenzymes (EC 2.7.2.3), one in the chloroplast and the other in the cytosol. The two isoenzymes are antigenically related, but can be distinguished on the basis of their isoelectric point (p1) values and on the basis of their affinity for magnesium and other substrates (Anderson and Advani, Plant Physiol. 45:583-585 (1970); Kopke-Secundo et al., Plant Physiol. 93:40-47 (1990)).

Three different glyceraldehyde 3-phosphate dehydrogenase (GAPDH (EC 1.2.1.13)) enzymes are found in eukaryotic cells (Pupillo and Faggiani, Arch. Biochem. Biophys. 194:581-592 (1979); Iglesias, Biochem. Educ. 18:2-5 (1990)). In higher plants there are two chloroplast GAPDH subunits: GapA (36 kDa) and GapB (42 kDa). The functional enzyme is reported to be a tetramer with either an A₄ or an A₂B₂ subunit structure (Cerff, In: Methods in Chloroplast Molecular Biology, Edelman, ed., Elsevier Press, Amsterdam: 683 (1982)). Sequence analysis of tobacco cDNA clones encoding the GapA and GapB subunits has revealed that they share homologues (Shih et al., Cell 47:73-83 (1986)). The three-dimensional structure of GADPH from both eukaryotes and prokaryotes has been studied, and it has been reported that the initial binding of the NAD coenzyme triggers a number of structural changes (Skarzynski and Wonacott, J. Mol. Biol. 203:1097-1118 (1988)).

Chloroplastic triose phosphate isomerase (TPI (EC 5.3.1.1)) is a homodimer with a subunit molecular weight of about 27 kDa (Pichersky and Gottlieb, Plant Physiol. 74:340-347 (1984)). The chloroplastic enzyme is reported to be distinguishable from the cytosolic enzyme by isoelectric focusing and peptide digestion mapping (Pichersky and Gottlieb, Plant Physiol. 74:340-347 (1984); Kurzok and Feierabend, Biochim. Biophys. Acta 788:222-233 (1984)). TPI, like several other RPP cycle enzymes, binds the substrate in a pocket, which is reported to be closed by a flexible loop which acts to shield the substrate from attack by water. Even though the active site is formed by residues from one subunit, the second subunit helps to exclude water from the active site domain.

Two reactions of the RPP cycle involve aldolase (EC 4.1.2.13), and both are catalyzed by the same enzyme, which is a tetramer of the 38 kDa subunit. It has been reported that each subunit of aldolase has a beta/alpha barrel structure (Sygusch et al., Proc. Natl. Acad. Sci. (U.S.A.) 84:7846-7850 (1987)) and that the C-terminal region covers the active site pocket, which is in the barrel and regulates access to the active site pocket.

Fructose-1,6-bisphosphatase (FBPase) (EC 3.1.3.11) is a homotetramer with a molecular weight of about 160 kDa. The amino acid sequence is reported to be highly conserved (Raines et al., Nucleic Acid Res. 16:7931-7942 (1988)). In both wheat and spinach, 12 extra amino acid residues have been identified that have been reported to be involved in the regulation by light via the ferredoxin/thioredoxin system (Raines et al., Nucleic Acid Res. 16:7931-7942 (1988); Marcus et al., Proc. Natl. Acad. Sci. (U.S.A.) 85:5379-5383 (1988)).

Transketolase (EC 2.2.1.1) (152 kDa tetramer) is found in cytosolic and chloroplastic forms. These forms are reported to have similar properties except for their response to Mg²⁺ (Feierbend and Gringel, Zeitschrift fur Pflanzenphysiol. 110:247-258 (1983); Murphy and Walker, Planta 155:316-320 (1982)).

Sedoheptulose-1,7-bisphosphate phosphatase (SBPase (EC 3.1.3.37)) is not reported to have a cytosolic counterpart and is reported to be found only in the chloroplast. The enzyme is reported to be a homodimer with a subunit molecular weight of 35-38 kDa (Nishizawa and Buchanan, J. Biol. Chem. 256:6119-6126 (1981); Cadet and Meunier, Biochem. J. 253:243-248 (1988)).

D-ribulose-5-phosphate-3-epimerase (EC 5.1.3.1) has been reported in animals as a homodimer with a subunit molecular weight of 23 kDa (Karmali et al., Biochem. J. 211:617-623 (1983)).

Ribose-5-phosphate isomerase (EC 5.3.1.6) has been purified from tobacco and spinach and is reported to be a homodimer with a subunit molecular weight of 26 kDa (Rutner, Biochemistry 9:178-184 (1970); Babadzhanova and Bakaeva, Biokhimiya 53:134-140 (1987)).

ii. Regulation of C3 Photosynthesis

The regulatory properties of the RPP cycle have been reported by Edwards and Walker, C3-C4: Mechanism and Cellular and Environmental Regulation of Photosynthesis, Blackwell Scientific Publications, Oxford, (1983); Leegood, Photosynthesis Res. 6:247-259 (1985); Woodrow, Biochim. Biophys. Acta 851:181-192 (1986). The conservation of phosphate is reported to play a role in the regulation of C3 photosynthesis, as a change in the level of any phosphorylated intermediate is balanced by an equal and opposite change in terms of phosphate elsewhere in the cycle (Woodrow, Biochim. Biophys. Acta. 851:181-192 (1986); Fell and Sauro, Eur. J. Biochem. 148:555-561 (1985)). Therefore, changes in the activity of any of the RPP cycle enzymes can affect both the substrate concentration and activities of other enzymes in the chloroplast.

iii. The C4 Pathway of Carbon Assimilation

In the C4 pathway, CO₂ is concentrated in bundle sheath cells at the site of the RPP cycle initiated by ribulose biphosphate carboxylase. C3 photosynthesis is documented to be the only mode of carbon assimilation in algae, bryophytes, pteridophytes, gymnosperms, and the majority of angiosperm families. Only about 10 families of known monocots and dicots have been reported to possess the C4 pathway of photosynthesis, these include, for example, maize, sorghum, sugar cane, etc. The C4 pathway has been reviewed by, for example, Edwards et al., In: CO₂ Metabolism and Productivity of Plants, Burris and Black, eds., University Park Press, Baltimore, Md., p. 83 (1976); Hatch, Biochim. Biophys. Acta 895:81-106 (1987); Ashton et al., In: Methods In Plant Biochemistry, Vol. 3, Academic Press Limited, New York, p. 39 (1990). A feature reported to be common to the enzymes in the C4 pathway is that their activities are 15-100 times higher compared to those reported in C3 plants. For example, adenylate kinase and pyrophosphatase activities are reported to be 20-50 times higher in C4 plants than in C-3 plants. Adenylate kinase and pyrophosphatase are largely located in the mesophyll chloroplast together with pyruvate Pi dikinase (Slack et al., Biochem. J. 114:489-498 (1969)).

In certain plant types (e.g., maize, sorghum and sugar cane), CO₂ is initially assimilated in mesophyll cells (with phosphoenolpyruvate (“PEP”) acting as a primary acceptor of CO₂) as oxaloacetate, which is reduced to malate by NADP-malate dehydrogenase. It has been reported that malate is moved to bundle sheath cells. In the chloroplast of bundle sheath cells, malate is decarboxylated by NADP-malic enzyme (malate formers) giving rise to pyruvate, and releasing CO₂ and NADPH. NADPH can be cycled back to NADP by coupling to PGA reduction in the RPP cycle. The carbon formed moves back to the mesophyll cells where it is converted to PEP by pyruvate Pi dikinase.

Plants of the PEP carboxykinase type are reported to have higher activities of aspartate and alanine aminotransferases than the malate formers. Such plants are reported to be aspartate formers rather than malate formers. In aspartate formers, the activity of PEP carboxykinase is reported to be higher and the activity of NADP-malic enzyme is reported to be lower (Edwards and Black, In: Photosynthesis and Photorespiration, Hatch et al., eds., Wiley Interscience, New York, p. 153 (1971)). It has been reported that the PEP carboxykinase is located in the cytosol of bundle sheath cells.

This group of C4 plants is not reported to contain either high levels of NAD-malic enzyme activity or high levels of PEP carboxykinase. It has been reported by Hatch and Kagawa (Aust. J. Plant Physiol. 1:357-369 (1974)) that these plants contain high NAD-malic enzyme activity in mitochondria and that the number of mitochondria in these plants may be increased by a factor of 34.

iv. Enzymes Involved in the C4 Pathway

Phosphoenolpyruvate carboxylase (PEP carboxykinase (EC 4.1.1.31)) is reported to initiate the carboxylative phase of the C4 metabolic route by catalyzing the irreversible beta-carboxylation of PEP. The reaction utilizes a divalent metal ion (e.g., Mg²⁺) as a cofactor. In C4 plants, PEP carboxykinase is reported to play a role in catalyzing the initial fixation of atmospheric CO₂ in the cytoplasm of mesophyll cells (O'Leary, Annu. Rev. Plant Physiol. 33:297-315 (1982); Andreo et al., FEBS Lett. 213:1-8 (1987)). PEP carboxykinase from C4 plants is reported to be a homotetramer with molecular weight of 400 kDa (O'Leary, Annu. Rev. Plant Physiol. 33:297-315 (1982); Andreo et al., FEBS Len. 213:1-8 (1987)). Each subunit is reported to contain at least one substrate-binding site. The monomeric form is reported to be inactive (Wagner et al., Eur. J. Biochem. 173:561-568 (1988); Walker et al., Plant Physiol. 80:848-855 (1986); Wagner et al., Eur. J. Biochem. 164:661-666 (1987).

In C4 plants, PEP carboxykinase is reported to be allosterically regulated. Glucose-6-phosphate, triose-phosphate and Pi are reported to be activators, and malate is reported to be an inhibitor of enzyme activity. C4 PEP carboxykinase is also reported to be subject to light regulation. Responses to light/dark involve a post-translational modification of the enzyme (Jiao and Chollet, Plant Physiol. 95:981 (1991)). The PEP carboxykinase is phosphorylated, during the light phase, at a serine residue close to the N-terminal region of the enzyme (Ser-15 in maize) (Jiao and Chollet, Plant Physiol. 95:981 (1991)). The phosphorylation is reported to be catalyzed by a soluble protein-serine kinase. The phosphorylated form of PEP carboxykinase is reported to be less sensitive to malate inhibition.

NADP-dependent malate dehydrogenase (NADP-MDHase (EC 1.1.1.82)) is reported to be located in the chloroplast of mesophyll cells and is reported to reduce oxaloacetate (OAA) by using photosynthetically generated NADPH. The native enzyme is reported to be a dimer composed of a nuclear-encoded subunit of molecular mass 42 kDa (Jenkins et al., Plant Sci. 45:1-7 (1986); Kagawa and Bruno, Arch. Biochem. Biophys. 260:674-695 (1988)). In C4 plants, NADP-MDHase is reported to have an alkaline pH optimum and the reduction of OAA is reported to be inhibited by NADP+. NADP-MDHase is reported to be light regulated with the enzyme active during the light phase and inactive during the dark phase. The activation mechanism involves reversible thiol/disulfide interchanges mediated by ferredoxin and thioredoxin m. The reaction is promoted under conditions of high NADPH:NADP+ ratio in the chloroplast stroma.

Aspartate aminotransferase (EC 2.6.1.1) is a cytoplasmic enzyme that converts OAA and glutamate into aspartate and alpha-ketoglutarate (alpha-KG) in mesophyll cells (Taniguchi et al., Arch. Biochem. Biophys. 282:427-432 (1990); Rastogi et al., J. Bacteriol. 173:2879-2887 (1991); Reynolds et al., Plant Mol. Biol. 19:465-472 (1992); Kirk et al., Plant Physiol. 105:763-764 (1994); Schultz et al., Plant J. 7:61-75 (1995)). Aspartate is exported into bundle sheath cells where decarboxylation takes place. Aspartate aminotransferase is reported to be present in aspartate forming C4 plants.

Alanine aminotransferase (EC 2.6.1.2) is reported to be present in C4 plants of the NAD-dependent malic acid enzyme (NAD-ME) type and interconverts in a reversible reaction the metabolites pyruvate and alanine in the cytoplasm of both mesophyll and bundle sheath cells (Son et al., Plant Mol. Biol. 20:705-713 (1992); Umemura et al., Biosci. Biotechnol. Biochem. 58:283-287 (1994)). The amino acid alanine is a metabolite transported in this C4 subtype.

NADP-dependent malic enzyme (NADP-ME (EC 1.1.1.40)) is reported to be present in NADP-ME type C4 plants and is located in the chloroplasts of bundle sheath cells. NADP-ME catalyses the conversion of malate into pyruvate and CO₂ in the presence of NADP+. This reaction is reported to require a metal ion (Ashton et al., In: Methods in Plant Biochemistry, Lea, ed., Academic Press, New York, p. 39 (1990); Leegood and Osmond, In: Plant Physiology, Biochemistry and Molecular Biology, Dennis and Turpin, eds., Wiley & Sons, Inc., New York, p. 274 (1990)). The NADP-ME enzyme in C4 plants is reported to comprise a single subunit with molecular weight of 62 kDa. At least two plastidic isoforms of NADP-ME, “dark” form and “light” form (the light form is also know as the “green” form), have been reported in maize leaves (Andreo et al., In: Proceedings of the International Congress on Photosynthesis, Montepelier, France, Mathis (ed.), Kluwer Academic Publishers, Amsterdam, (1995)). The dark form of the NADP-ME, which is present mainly in etiolated maize leaves, has a molecular weight of 72 kDa and a lower specific activity compared to the “green” form of NADP-ME (62 kDa) found in green leaves (Andreo et al., In: Proceedings of the International Congress on Photosynthesis, Montepelier, France, Mathis, ed., Kluwer Academic Publishers, Amsterdam, (1995)). The “green” form of NADP-ME appears to be enhanced by light. The dark form of the enzyme resembles the NADP-MEs found in C-3 plants in both photosynthetic and nonphotosynthetic tissues.

NAD-dependent malic enzyme (NAD-ME (EC 1.1.1.39)) is reported to be located in the mitochondria where it catalyzes the NAD-dependent decarboxylation of malate in the presence of a divalent cation (e.g., Mg²⁺). NAD-ME is reported to be ineffective in the decarboxylation of OAA (Artus and Edwards, FEBS Lett. 182:225-233 (1985). NAD-ME is reported to comprise two subunits (alpha and beta) which differ in molecular weights (58 and 62 kDa, respectively).

In C4 plants of the PEP carboxykinase (EC 4.1.1.49) type, aspartate is converted into OAA in bundle sheath cells and ketoacid is decarboxylated by cytoplasmic PEP carboxykinase. PEP carboxykinase is reported to have a requirement for Mn²⁺ and a preference for ATP (Ashton et al., In: Methods in Plant Biochemistry, Lea (ed.), Academic Press, New York, p. 39 (1990)). The native enzyme is reported to be a homohexamer with a molecular weight of 380 kDa (subunit molecular weight of 64 kDa). PEP carboxykinase enzyme is reported to be inhibited by the metabolites 3PGA, fructose-6-phosphate, fructose 1,6 bisphosphate and DHAP.

In all three subtypes of C4 plants, regeneration of PEP from pyruvate takes place in mesophyll chloroplasts by the reaction catalyzed by pyruvate Pi dikinase (PPDKase (EC 2.7.9.1)). This is a regulatory step in the C4 pathway (Hatch, Biochim. Biophys. Acta 895:81-106 (1987); Ashton et al., In: Methods in Plant Biochemistry, Lea (ed.), Academic Press, New York, p. 39 (1990)). PPDKase is a homotetrameric protein with a molecular weight of about 390 kDa (Ashton et al., In: Methods in Plant Biochemistry, Lea (ed.), Academic Press, New York, p. 39 (1990)). PPDKase is reported to be inactivated by cold temperatures and the absence of Mg²⁺ and is activated in the light period and inactivated in the dark period ((Ashton et al., In: Methods in Plant Biochemistry, Lea (ed.), Academic Press, New York, p. 39 (1990)). Activation by light of PPDKase is a result of dephosphorylation and the switch to inactive dark form involves phosphorylation.

Pyrophosphatase (inorganic pyrophosphatase (EC 3.6.1.1)) promotes the reaction catalyzed by the enzyme pyruvate Pi dikinase in the direction of PEP synthesis through hydrolysis of PPi (Jiang et al., Arch. Biochem. Biophys. 346:105-112 (1997); Mitchell et al., Can. J. Microbiol. 43:734-743 (1997)). Pyrophosphatase has been isolated from potato (du Jardin et al., Plant Physiol. 109:853-860 (1995)) and Arabidopsis (Kieber and Signer, Plant Mol. Biol. 16:345-348 (1991)).

Ribose-5-phosphate kinase (EC 2.7.1.19) is reported to be found in photosynthetic organisms possessing the C-4 pathway. This homodimeric enzyme has a subunit molecular weight of 39.2 kDa (Roeslier and Ogren, Nucleic Acid Res. 16:7192 (1988); Milanez and Mural, Gene 66:55-63 (1988)). The N-terminal region seems to be involved in the regulation of catalytic activity. Cys¹⁶ may form a part of the ATP-binding region. Lys⁶⁸ has also been implicated in ATP binding (Miziorko et al., J. Biol. Chem. 265:3642-3647 (1990)).

B. Carbohydrate Metabolism

1. Glycolysis and Gluconeogenesis Pathways

i. The Glycolysis Pathway

Glycolysis plays a role in supplying energy to most organisms. Glycolysis, although it is per se anaerobic, is reported to be the primary source of carbon for respiration in plants via the citric acid cycle (Plaxton, Annu. Rev. Plant Physiol. Plant Mol. Biol. 47:185-214 (1996)). Under conditions of low oxygen, pyruvate generated from glycolysis can be converted into ethanol or lactate via fermentation. In addition to the production of energy, glycolysis can produce intermediates for formation of essential molecules such as amino acids (Plaxton, Annu. Rev. Plant Physiol. Plant Mol. Biol. 47:185-214 (1996); Salisbury and Ross, Plant Physiol. Wadsworth Pub. Co., Belmont, Calif. (1978)). In plants, glycolysis has been reported to take place in both the cytosol and plastids, using isozymes encoded by separate nuclear genes (Plaxton, Annu. Rev. Plant Physiol. Plant Mol. Biol. 47:185-214 (1996); Dennis and Miernyk, Annu. Rev. Plant Physiol. 33:27-50 (1982); Stitt and apRees, Phytochem. 18:1905-1911 (1979)). It has been reported that metabolites can be passed between the glycolysis and gluconeogenesis pathways via transporters (Plaxton, Annu. Rev. Plant Physiol. Plant Mol. Biol. 47:185-214 (1996)). It has also been reported that the maize phosphate translocator in C4 plants readily transports 2-phosphoglycerate and phosphoenolpyruvate, whereas the translocator in spinach, a C3 plant, transports these molecules very poorly (Gross et al., Planta 180:262-271 (1990)). Genes representing many of the enzymes of glycolysis have been reported.

Glycolysis may begin with glucose, fructose, or a glucose phosphate, all of which are eventually converted to fructose-6-phosphate. If glycolysis is begun with glucose or glucose-1-phosphate, glucose-6-phosphate is produced as an intermediate. Glucose may be phosphorylated to glucose-6-phosphate by hexokinase (EC 2.7.1.1) in an irreversible reaction requiring ATP and Mg²⁺ (Brownleader et al., In: Plant Biochemistry, Academic Press, New York pp. 111-141 (1997)). A hexokinase cDNA isolated from Arabidopsis thaliana has been reported (Dai et al., Plant Physiol. 108:879-880 (1995)).

Phosphoglucomutase (EC 5.4.2.2) catalyzes the conversion of glucose-1-phosphate to glucose-6-phosphate (Tetlow et al., Biochem. Soc. Trans. 25:468S (1997)). A plastidic phosphoglucomutase cDNA sequence has been reported from Spinacia oleracea (L.) (Penger et al., Plant Physiol. 105:1439-1440 (1994)). Phosphoglucomutase isozyme variants have been studied in maize. (Stuber and Goodman, Biochem. Genet. 21:667-689 (1983)).

Hexose phosphate isomerase (EC 5.3.1.9) catalyzes the conversion of glucose-6-phosphate to fructose-6-phosphate. cDNAs, isolated from several species including maize, have been reported (Lal and Sachs, Plant Physiol 108:1295-1296 (1995)). Fructose-6-phosphate can also be produced by the phosphorylation of fructose via fructokinase (EC 2.7.1.4). A fructokinase cDNA clone, isolated from potato, has been reported (Smith et al., Plant Physiol. 102:1043 (1993)).

Phosphofructokinase catalyzes the first reported step in glycolysis by converting fructose-6-phosphate to fructose-1,6-bisphosphate (Turner and Turner, In: Biochemistry of Plants—A Comprehensive Treatise, Vol. 2, pp. 279-316 (1980)). Two types of phosphofructokinases can catalyze this reaction: an ATP-dependent phosphofructokinase, known as ATP-dependent fructose-6-phosphate 1-phosphotransferase (EC 2.7.1.11), also known as PFK, which catalyzes an irreversible and regulated reaction and a pyrophosphate-dependent form, known as fructose-6-phosphate:pyrophosphate phosphotransferase (EC 2.7.1.90), also known as PFP, which catalyzes a freely reversible reaction stimulated by fructose-2,6-bisphosphate (apRees, In: Encyclopedia of Plant Physiology Vol. 18 pp. 391-417, (1985); Stitt, Annu. Rev. Plant Physiol. Plant Mol. Biol. 41:153-185, (1990)). Phosphofructokinase (PFK) has been reported to be the primary enzyme catalyzing the conversion of fructose-6-phosphate to fructose-1,6-bisphosphate in glycolysis (Brownleader et al., In: Plant Biochemistry Academic Press, New York pp. 111-141(1997)). Reports on transgenic potato tubers indicate that phosphofructokinase (PFP) can catalyze a net glycolytic flux (Kruger and Scott, Biochem. Soc. Trans. 22:904-909, (1994); Hajirezaei et al., Planta 192:16-30 (1994)). Reports have indicated that transgenic potato plants with approximately 1% of normal activity of phosphofructokinase (PFP) in tubers had no detectable changes in phenotype other than a small increase in sucrose and a decrease in starch in the tubers. The results of the report indicate that the phosphofructokinase (PFK) is normally present is sufficient to maintain flux through the glycolytic pathway and that phosphofructokinase (PFP) is present in excess (Hajirezaei et al., Planta 192:16-30 (1994)). Phosphofructokinase (PFK) is inhibited by phosphoenolpyruvate; this inhibition of phosphofructokinase can be relieved by Pᵢ (orthophosphate ion) (Plaxton, Ann. Rev. Plant Physiol. Plant Mol. Biol. 47:185-214 (1996)). Phosphofructokinase (PFK) catalyzes a non-equilibrium reaction and has been reported to be the first of two regulatory points in glycolysis (apRees, In: The Biochemistry of Plants, Vol. 3 pp. 1-42 (1980)). Fructose-6-phosphate:pyrophosphate phosphotransferase (PFP) has been cloned from potato (Carlisle et al., J. Biol. Chem. 265:18366-18371 (1990)) and castor bean (Todd et al., Gene 152:181-186 (1995)).

Fructose-1,6-bisphosphate aldolase (EC 4.1.2.13) catalyzes the breakdown of fructose-1,6-bisphosphate. The breakdown of fructose-1,6-bisphosphate by fructose-1,6-bisphosphate aldolase yields two three-carbon molecules, dihydroxyacetone phosphate and glyceraldehyde-3-phosphate. Aldolase clones, isolated from maize (Kelley and Tolan, Plant Physiol. 82:1076-1080 (1986)) and rice (Hidaka et al., Nucl. Acids Res. 18:3991 (1990)), have been reported.

Triose phosphate isomerase (EC 5.3.1.1) converts dihydroxyacetone phosphate to glyceraldehyde-3-phosphate. Because the conversion of dihydroxyacetone phosphate results in a six-carbon molecule becoming two molecules with three carbons each, the following reactions produce two molecules per initial hexose molecule. Sequences of triose phosphate isomerase have been reported from maize (Marchionni and Gilbert, Cell 46:133-141 (1986)) and rice (Xu and Hall, Plant Physiol. 101:683-687 (1993)).

Glyceraldehyde-3-phosphate dehydrogenase (EC 1.2.1.12) catalyzes the conversion of glyceraldehyde-3-phosphate into glycerate-1,3-bisphosphate. It has been reported that maize has cytosolic and plastid forms of this enzyme (Russell and Sachs, Mol. Gen. Genet. 229:219-228 (1991)) as does Arabidopsis thaliana (Shih et al., Gene 104:133-138 (1991)).

Glycerate-3-phosphate kinase (EC 2.7.2.3), also known as phosphoglycerate kinase, converts glyceraldehyde-1,3, bisphosphate to glycerate-3-phosphate. The conversion of glyceraldehyde-1,3, bisphosphate to glycerate-3-phosphate produces one molecule of ATP and requires Mg²⁺. Longstaff et al. have reported both cytosolic and plastidic forms of this enzyme from wheat (Longstaff et al., Nucleic Acids Res. 17:6569-6580 (1989)).

Glycerate-P-mutase (EC 5.4.2.1), also known as phosphoglycerate mutase, converts glycerate-3-phosphate to glycerate-2-phosphate. In plants, glycerate-P-mutase is cofactor independent. Glycerate-P-mutase has been reported from maize (Grana et al., J. Biol. Chem. 267:12797-12803 (1992)), tobacco, and castor bean (Huang et al., Plant Mol. Biol. 23:1039-1053 (1993)).

Enolase (EC 4.2.1.11), also known as phosphopyruvate hydratase, catalyzes the conversion of glycerate-2-phosphate to phosphoenolpyruvate and H₂O. Enolase is reported to require Mg²⁺ for its catalytic activity. Enolase has been reported from maize (Lal et al., Plant Mol. Biol. 16:787-795 (1991)), castor bean (Blakeley et al., Plant Physiol. 105:455-465 (1994)), tomato and Arabidopsis thaliana (van der Straeten et al., Plant Cell 3:719-735 (1991)).

Pyruvate kinase (EC 2.7.1.40) catalyzes conversion of phosphophenolpyruvate to pyruvate yielding a molecule of ATP. Pyruvate kinase has been reported to be encoded by multiple genes in certain plants. Certain isozymes have been reported to be targeted to specific plastid types. The conversion of phosphophenolpyruvate to pyruvate can also be catalyzed by phosphoenolpyruvate phosphatase (EC 3.1.3.2), also known as PEPase. It has been reported that in transgenic tobacco plants lacking cytosolic pyruvate kinase in their leaves, no change in aboveground phenotype or metabolism was observed. This report indicates that other pathways, such as that using PEPase, are capable of bypassing the need for pyruvate kinase (Gottlob-McHugh et al., Plant Physiol. 100:820-825 (1992)). The pyruvate kinase reaction is a non-equilibrium reaction and has been reported to be a second of two regulatory points in glycolysis (apRees, In: The Biochemistry of Plants, Vol. 3 pp. 142, 1980). Pyruvate kinase has been reported from a number of plants including tobacco, castor bean (Blakeley et al., Plant Mol. Biol. 27.79-89 (1995)) and potato (Cole et al., Gene 122:255-261 (1992)).

In the presence of oxygen, pyruvate from glycolysis can enter the citric acid cycle. Pyruvate is first converted to acetyl-coA by a pyruvate dehydrogenase enzyme complex, consisting of pyruvate dehydrogenase (EC 1.2.4.1), dihydrolipoamide-s-acetyltransferase (EC 2.3.1.12), and dihydrolipoamide dehydrogenase (EC 1.8.1.4). Pyruvate dehydrogenase and dihydrolipoamide-s-acetyltransferase subunit genes have been reported from Arabidopsis thaliana (Luethy et al., Biochem. Biophys. Acta. 1187:95-98, (1994), Guan et al., J. Biol. Chem. 270:5412-5417 (1995)). A dihydrolipoamide dehydrogenase gene has been isolated from pea (Turner et al., J. Biol. Chem. 267:7745-7750 (1992)).

In the absence of oxygen, pyruvate from glycolysis can undergo fermentation by using one of two pathways. In one pathway, lactate dehydrogenase (EC 1.1.1.27) catalyzes the conversion of pyruvate to lactate. In the other pathway, a two step process is involved. First, pyruvate is converted to acetaldehyde by pyruvate decarboxylase (EC 4.1.1.1). Next, acetaldehyde is converted to ethanol by alcohol dehydrogenase (EC 1.1.1.1). It has been reported that pyruvate decarboxylase activity is favored at low pH, so that as lactate is produced, lactate dehydrogenase activity decreases while pyruvate decarboxylase activity increases (Davies et al., Planta 118:297-310 (1974)). Gene sequences representing alcohol dehydrogenase, lactate dehydrogenase, and pyruvate decarboxylase have been cloned from maize (Dennis et al., Nucl. Acid Res. 12:3983-4000 (1984); Good and Paetkau, Plant Mol. Biol. 19:693-697 (1992); Kelley et al., Plant Mol. Biol. 17:1259-1261 (1991)).

ii. The Gluconeogenesis Pathway

Gluconeogenesis takes place primarily in germinating oil seeds and has been reported to be the predominant metabolic activity during germination of oil seeds (apRees, In: Encyclopedia of Plant Physiology, Vol. 18 pp. 391-417 (1985); apRees, In: The Biochemistry of Plants, Vol. 3 pp. 1-42 (1980)). Gluconeogenesis provides a mechanism for the breakdown of stored lipids into sugars. Developing seedlings may utilize sugars which result from this breakdown of stored lipids.

Gluconeogenesis has been reported to begin with oxaloacetate. Oxaloacetate has been reported to be produced from succinate with no net loss of carbon. apRees has reported that the conversion from fatty acids to succinate can occur in glyoxysomes and that the conversion of succinate to oxaloacetate occurs in the mitochondria. Reports also indicate that the remaining reactions of gluconeogenesis may occur in the cytosol (apRees, In: The Biochemistry of Plants, Vol. 3 pp. 142 (1980)).

Gluconeogenesis reactions are not the exact reverse of glycolysis. Glycolysis and gluconeogenesis differ in two ways. There are two reported irreversible reactions in glycolysis which are catalyzed by pyruvate kinase and phosphofructokinase. These enzymes are not utilized in gluconeogenesis. Gluconeogenesis begins with oxaloacetate that is not a substrate reported to be associated with glycolysis.

PEP carboxykinase (EC 4.1.1.49) catalyzes the conversion of oxaloacetate to phosphoenolpyruvate and CO₂ in the first reported reaction of gluconeogenesis. PEP carboxykinase has been reported from the Urochloa panicoides (Finnegan and Burnell, Plant Mol. Biol. 27:365-376 (1995)) and from cucumber (Kim and Smith, Plant Mol. Biol. 26:423-434 (1994)). Phosphoenolpyruvate is converted to fructose-1,6-bisphosphate in six steps utilizing enolase, phosphoglycerate mutase, phosphoglycerate kinase, glyceraldehyde-3-phosphate dehydrogenase, triose phosphate isomerase, and aldolase in the reverse order as in glycolysis.

The fructose-1,6-bisphosphate to fructose-6-phosphate reaction is catalyzed by fructose-1,6-bisphosphatase (EC 3.1.3.11). Fructose-1,6-bisphosphatase cDNA has been isolated from spinach (Martin et al., Plant Mol. Biol. 32:485-491 (1996); Hur et al., Plant Mol. Biol. 18:799-802 (1992)), oilseed rape (Laroche et al., Plant Physiol. 108:1335-1336 (1995); Rodriguez-Suarez and Wolosiuk, Plant Physiol. 103:1453-1454 (1993)), pea (Jacquot et al., Eur. J. Biochem. 229:675-681 (1995); Dong et al., Plant Physiol. 107:313-314 (1995); Carrasco et al., Planta 193:494-501 (1994)), and Arabidopsis thaliana (Horsnell and Raines, Plant Mol. Biol. 17:185-186 (1991)).

Gluconeogenesis has been reported to be capable of converting about 70% of the carbon from fat to sucrose. The majority of the loss of carbon in the conversion of fat to sucrose has been reported to be due to the CO₂ released in the PEP carboxykinase reaction. The efficiency of gluconeogenesis may indicate that little carbon is lost to respiration. Some carbon, however, may be respired for use in biosynthetic reactions in the seedling (apRees, In: The Biochemistry of Plants, Vol. 3 pp. 142 (1980)).

2. Sucrose Metabolism

Carbon fixed during photosynthesis is either retained in the chloroplast and converted to a storage carbohydrate, for example, starch, or it is transferred to the cytosol in the form of triose phosphates and converted to sucrose. The newly synthesized sucrose in source tissues is a major transported form of reduced carbon in higher plants and can be either metabolized into other carbohydrates, stored in the vacuole or exported to other plant tissues. Plant tissues where sucrose is synthesized, such as leaves, are often referred to as ‘source’ tissues. Translocated sucrose is retained in ‘sink’ tissues (such as expanding leaves, growing seeds, flowers, roots or tubers, and fruit) and may be assimilated, or further metabolized to sustain cell maintenance or fuel growth, or be converted to alternative storage compounds (e.g., starch, fats). The relative type and size of these carbohydrate pools vary during tissue development, between different plant species, and within the same species subject to different environmental conditions. Such differences are reported to affect the yield and quality of agricultural produce.

Sucrose synthesis and catabolism are reported to be highly coordinated and regulated processes that may also be coordinately regulated with other dedicated metabolic pathways in a particular plant, plant organ or cell type. Sucrose synthesis is reported to be coordinately regulated with starch metabolism and photosynthesis in green ‘source’ plant tissues. Sucrose supply by transport mechanisms to actively growing ‘sink’ tissues is reported to be coordinated with plant development. In growing sink tissues, the supply of carbohydrate is reported to be important to other metabolic pathways and physiological processes including respiration, starch biosynthesis, cell wall biogenesis, lipid and protein biosynthesis. Sucrose synthesis and/or transport is also reported to play a role in the carbohydrate capacity that is available to growing fruits and seeds. Sucrose resynthesis during seed germination is reported to play a role in seedling vigor and agronomic stand establishment in many plant species during early plant development.

In many plant species, enzymes of pathways involved in sucrose metabolism can play a role in plant physiology and plant growth and development. Compartmentation and temporal regulation of genes and enzymes of sucrose metabolic pathways can allow multiple pathways to utilize sucrose as a common metabolite. Flux through a particular sucrose metabolic pathway can define the utilization of sucrose in any tissue or developmental stage. Sucrose and its metabolite products have been reported to play a role in gene regulation and expression of the sucrose pathway and other metabolic pathways in plants.

Reviews on sucrose metabolism in plants include Avigad, In: Encyclopedia of Plant Physiology, Vol. 13A, Loewus and Tanner (eds.), Springer Verlag, Heidelberg, pp. 217-347 (1982); Hawker, In: Biochemistry of Storage Carbohydrates in Green Plants, Dey and Dixon (eds.), Academic Press, London, 1-51 (1985); Huber et al., In: Carbon Partitioning Within and Between Organisms, Pollock et al. (eds.), Bios Scientific, Oxford, 1-26 (1992); Stitt et al., In: Biochemistry of Plants, Vol. 10, Hatch and Boardman (eds.), Academic Press, New York, 327-407 (1987); Quick and Schaffer, In: Photoassimilate Distribution In: Plants And Crops, Zamski and Schaffer (eds.), Marcel Dekker Inc., New York, 115-156 (1996).

The synthesis of sucrose precursors (triose and hexose phosphates) is derived from either photosynthetic CO₂ fixation or degradation of previously deposited storage reserves. One substrate for sucrose synthesis in photosynthetic tissues is three carbon sugar phosphates. These are exported from the chloroplast during photosynthesis, predominantly in the form of triose phosphates. The pool of triose phosphates, dihydroxyacetone phosphate (“DHAP”), and glyceraldehyde-3-phosphate (“GAP”), is maintained at equilibrium within the cytoplasm by triose phosphate isomerase (EC 5.3.1.1). A subsequent reaction involves an aldol condensation of DHAP and GAP, catalyzed by the enzyme fructose 1,6-bisphosphate aldolase (often called aldolase) (EC 4.1.2.13) to form fructose 1,6-bisphosphate (“F1,6BP”). Fructose-1,6-bisphosphatase (“FBPase”) (EC 3.1.3.11) catalyzes the cleavage of phosphate from the C1 carbon of fructose-1,6-bisphosphate to form fructose-6-phosphate (“F6P”). This reaction is essentially irreversible and has been reported to represent the first committed step within the pathway of sucrose synthesis. The cytosolic FBPase has been reported to be subject to allosteric regulation and may serve to coordinate the rate of sucrose synthesis with that of photosynthesis. Fructose 2,6-bisphosphate (“F2,6BP”) is reported to be a regulator of FBPase (Black et al., In: Regulation of Carbohydrate Partitioning In Photosynthetic Tissue, Heath and Preiss (eds.), Waverly, Baltimore, 109-126 (1985); Stitt et al., In: Biochemistry Of Plants, Vol. 10, Hatch and Boardman (eds.), Academic Press, New York, 327-407 (1987)). The concentration of F2,6BP is reported to be controlled in plants by two enzymes, fructose-2,6-bisphosphatase (F2,6Bpase) (EC 3.1.3.46) and fructose-6-phosphate,2-kinase (F6P,2K) (EC 2.7.1.105) (Stitt, Annu. Rev. Plant Physiol. Plant Mol. Biol. 41:153-181 (1990)).

Glucose-6-phosphate (“G6P”) and glucose-1-phosphate (“G1P”) are reported to be maintained in equilibrium with the F6P pool by the action of phosphoglucoisomerase (“PGI”) (EC 5.3.1.9) and phosphoglucomutase (“PGM”) (EC 5.4.2.2), respectively. Uridine diphosphate glucose (“UDPG”) and pyrophosphate (“PPi”) are formed from uridine triphosphate (“UTP”) and G1P catalyzed by the enzyme UDPG-pyrophosphorylase (“UDPGase”) (EC 2.7.7.9). This reaction is reversible and net flux in the direction of sucrose synthesis is reported to require removal of its products, particularly PPi. A pyrophosphate-dependent proton pump, vacuolar H⁺-translocating-pyrophosphatase (EC 3.6.1.1), has been identified within the vacuolar membrane and has been reported to utilize pyrophosphate to sustain a proton gradient formed between these two compartments (Rea et al., Trends in Biol. Sci. 17:348-353 (1992)).

A pyrophosphate-dependent fructose-6-phosphate phosphotransferase (“PFP”) (EC 2.7.1.90) is also present in the cytoplasm and catalyzes the reversible production of F1,6BP and Pi from F6P and PPi. One reported function of PFP is to operate in a futile cycle with the cytosolic FBPase, and function as a “pseudopyrophosphatase” recycling PPi. Uridine diphosphate glucose is then combined with F6P to form sucrose-6-phosphate (“S6P”). This reaction is catalyzed by sucrose phosphate synthase (“SPS”) (EC 2.4.1.14). Attachment of UDP to the glucose moiety activates the C1 carbon atom of UDPG, which is necessary for the subsequent formation of a glycosidic bond in sucrose. In certain organisms, SPS is capable of using adenine diphosphate glucose (“ADPG”), instead of UDPG, as a substrate. The use of nucleotide biphosphate sugars is a feature of metabolic pathways leading to the production of disaccharides and polysaccharides. SPS is reported to be subject to allosteric and covalent regulation and, in conjunction with the cytosolic FBPase, reportedly serves to coordinate the rate of sucrose synthesis with the rate of photosynthesis. The reported final reaction in the pathway is catalyzed by sucrose-6-phosphate phosphatase (“SPPase” or “SPP”) (EC 3.1.3.24), which catalyzes the hydrolysis of S6P to sucrose. It has been reported that SPS and SPPase may associate to form a multienzyme complex, that the rate of sucrose-6-phosphate synthesis by SPS is enhanced in the presence of SPP, and that the rate of sucrose-6-phosphate hydrolysis by SPP is increased in the presence of SPS (Echeverria et al., Plant Physiol. 115:223-227 (1997)).

i. Sucrose Synthesis

Reviews describing fructose-1,6-bisphosphatase (“FBPase”, EC 3.1.3.11) include those by Hers and Van Shaftingen, Biochem J. 206:1-12 (1982), and Stitt, Annu. Rev. Plant Physiol. Plant Mol. Biol. 41:153-181 (1990). Two isoforms of FBPase are reported to exist in plants. The first isoform is associated with the plastid and occurs largely in photosynthetic plastids. The second isoform, located in the cytoplasm, is reported to be involved in both gluconeogenesis and sucrose synthesis (Zimmerman et al., J. Biol. Chem. 253:5952-5956 (1978); Stitt and Heldt, Planta 164:179-188 (1985). FBPase catalyzes an irreversible reaction in the direction of F6P synthesis in vivo and has been reported to represent the first committed step in the pathway of sucrose synthesis. The properties of the enzyme are reported to involve the action of several regulatory metabolites (Stitt et al., In: Biochemistry Of Plants, Vol. 10, Hatch and Boardman, eds., Academic Press, New York, 327-407 (1987)). The enzyme reportedly has a high affinity for its substrate F1,6BP, a requirement for Mg²⁺, a requirement for a neutral pH, is weakly inhibited (Km 2-4 μm) by adenosine monophosphate (AMP), and is strongly inhibited by the regulatory metabolite F2,6BP (Hers and Van Shaftingen, Biochem J. 206:1-12 (1982); Black et al., In: Regulation of Carbohydrate Partitioning In Photosynthetic Tissue, Heath and Preiss (eds.), Waverly, Baltimore, 109-126 (1985); Huber, Annu. Rev. Plant Physiol. 37:233-246 (1986); Stitt et al., In: Biochemistry Of Plants, Vol. 10, Hatch and Boardman (eds.), Academic Press, New York, 327-407 (1987)). F2,6BP is also an activator of PFP and reportedly plays a role in the regulation of gluconeogenetic and respiratory metabolism.

The concentration of F2,6BP is reportedly determined in plants by two enzymes, fructose-2,6-bisphosphatase (“F2,6BPase”) (EC 3.1.3.46) and fructose-6-phosphate,2-kinase (“F6P,2K”) (EC 2.7.1.105). A review of these enzymes is provided by Stitt, Annu. Rev. Plant Physiol. Plant Mol. Biol. 41:153-181 (1990). Regulation of the activity of the F1,6FBPase and the rate of sucrose synthesis is reported to be, at least in part, brought about by changes in the concentration of F2,6BP.

Sucrose phosphate synthase (SPS (EC 2.4.1.14)) catalyzes a reaction that is displaced from equilibrium in vivo in the direction of S6P synthesis and is reported as an essentially irreversible reaction in vivo (Stitt et al., In: Biochemistry Of Plants, Vol. 10, Hatch and Boardman (eds.), Academic Press, New York, 327-407 (1987); Lunn and Rees, Biochem. J. 267:739-743 (1990); U.S. Pat. No. 5,665,892). SPS has been purified from spinach and maize, and the amino acid and cDNA sequences have been published (Worrel et al., Plant Cell 3:1121-1130 (1991); Klein et al., Planta 190:498-510 (1993); Sonnewald et al., Planta 189:174-181 (1993)). The enzyme has a subunit molecular weight of 117 kDa from spinach (Klein et al., Planta 190:498-510 (1993); Sonnewald et al., Planta 189:174-181 (1993)) and pea (Lunn and Rees, Phytochem. 29:1057-1063 (1990)) and 135 kDa from maize (Worrel et al., Plant Cell 3:1121-1130 (1991)). The native enzyme reportedly exists as a tetramer (Walker and Huber, Plant Physiol. 89:518-524 (1988); Lunn and Rees, Phytochem. 29:1057-1063 (1990); Worrel et al., Plant Cell 3:1121-1130 (1991), although dimeric molecular weights have been reported (Klein et al., Planta 190:498-510 (1993)). Activity has been observed for SPS at both dimeric and tetrameric molecular weights (Sonnewald et al., Planta 189:174-181 (1993)).

SPS is located in the cytosol, has a neutral pH optimum, and has been detected in all plant tissues which undertake active sucrose synthesis. SPS is also reported to undertake active sucrose synthesis. An increase in abundance of the enzyme is has been reported during the development of leaves, germination of seeds and ripening of fruit. SPS has been reported to be subject to regulation by metabolites and is activated by G6P and is inhibited by Pi. Pi and GP6 are reported to act competitively at an allosteric site of the enzyme. In the presence of high Pi concentrations, the enzyme is phosphorylated which reduces activity of the enzyme. It has also been reported that light-induced photosynthesis increases the activity of SPS in crude extracts (Sicher and Kremer, Plant Physiol. 79:910-912 (1984), Sicher and Kremer, Plant Physiol. 79:695-698 (1985); Pollock and Housley, Ann. Bot. 55:593-596 (1985)). In addition, it has been reported that compounds altering the phosphate status of the leaf can simulate the effects of light. Feeding leaves mannose, which sequesters phosphate by its conversion to the non-metabolized mannose-6-P, has been reported to cause activation of SPS (Stitt et al., Planta 174:217-230 (1988)).

The phosphorylation and dephosphorylation of SPS is catalyzed by SPS-phosphatase and SPS-kinase, respectively (Huber et al., Plant Physiol. 99:1275-1278 (1992). Hydrolysis of sucrose-6-P to sucrose is catalyzed by sucrose-6-phosphatase (SPPase or SPP) (EC 3.1.3.24). The activity of both SPS and SPP is reported to be affected by a multienzyme complex between SPS and SPP (Echeverria et al., Plant Physiol. 115:223-227 (1997)).

Regulatory properties of SPS and FBPase are reported to coordinate the rate of sucrose synthesis with that of photosynthesis (Stitt, In: Plant Physiology, Biochemistry and Molecular Biology, Dennis and Turpin, eds., Singapore, London, 319-340 (1990)). When photosynthesis produces triose phosphate in excess of the rate of sucrose synthesis, a feed-forward activation of sucrose synthesis occurs. Triose phosphate crosses the chloroplast membrane in exchange for cytosolic Pi. Under these conditions, F6P,2-kinase activity is reduced and the inhibition of F2,6Bpase is decreased.

As cytosolic F2,6BP falls, F2,6BPase activity increases, and F6P levels increase. Hexose phosphate levels are reported to increase due to PGM and PGI, and with low Pi, activate SPS and F1,6BPase. Reduction in rate of photosynthesis must result in a deactivation of sucrose synthesis, which occurs through decreased cytosolic triose-P, increased Pi and ultimately increased F2,6BP concentration and reduced SPS activity (Stitt, Phil. Trans. R. Soc. Lond. B 342:225-233 (1993); Huber et al., Plant Physiol. 99:1275-1278 (1992); Neuhaus et al., Planta 181:583-592 (1990).

ii. Metabolic Pathways of Sucrose Catabolism

Sucrose can initially be cleaved by invertases (EC 3.2.1.26) or by sucrose synthases (EC 2.4.1.13). Invertases, which are classified as acid or alkaline in pH preference (Karuppiah et al., Plant Physiol. 91:993-998 (1989); Fahrendorf and Beck, Planta 180:237-244 (1990); Iwatsubo et al., Biosci. Biotech. Biochem. 56:1959-1962 (1992); Unger et al., Plant Physiol. 104:1351-1357 (1994); Avigad, In: Encyclopedia of Plant Physiology, Vol. 13A, Loewus and Tanner (eds.), Springer Verlag, Heidelberg, 217-347 (1982)), irreversibly cleave sucrose into glucose and fructose, both of which is usually phosphorylated for further metabolism. The invertase pathway usually is associated with rapidly growing sink tissues such as expanding leaves, expanding internodes, flower petals, and early fruit development (Avigad, In: Encyclopedia of Plant Physiology, Vol. 13A, Loewus and Tanner (eds.), Springer Verlag, Heidelberg, 217-347 (1982); Huber, Plant Physiol. 91:656-662 (1989); Morris and Arthur, Phytochem. 23:2163-2167 (1984); Hawker et al., Phytochem. 15:1441-1443 (1976); Schaffer et al., Plant Physiol. 69:151-155 (1987)).

Sucrose synthase carries out the kinetically reversible transglycosylation of sucrose and UDP into fructose and UDPG, requiring only the phosphorylation of fructose for additional metabolism. Polysaccharide biosynthesis in sink tissues may utilize a sucrose synthase mediated sucrose catabolism (Avigad, In: Encyclopedia of Plant Physiology, Vol. 13A, Loewus and Tanner, eds., Springer Verlag, Heidelberg, 217-347 (1982); Doehlert et al., Plant Physiol. 86:1013-1019 (1988); Dale and Housley Plant Physiol. 82:7-10 (1986)). Respiring tissues reportedly utilize either sucrose synthase or invertase metabolic pathways (Echeverria and Humphreys, Phytochem. 23:2173-2178 (1984); Uritani and Asahi, In: The Biochemistry of Plants Vol. 2, Davies (ed.), Academic Press, New York, 463-487 (1980)). Tissues that are undergoing respiration, starch biosynthesis, amino acid and fatty acid synthesis, rapid expansion or growth, and other cellular metabolism, can utilize several sucrose metabolic pathways which may be temporally or compartmentally regulated (Doehlert et al., Plant Physiol. 86:1013-1019 (1988); Doehlert, Plant Physiol. 78:560-567 (1990); Doehlert and Choury, In: Recent Advances in Phloem Transport and Assimilate Compartmentation, Bonnemain et al. (eds)., Ouest editions, Nantes, France, 187-195 (1991); Delmer and Stone, In: The Biochemistry of Plants, Vol. 14, Preiss (ed.), Academic Press, San Diego, 373-420 (1988); Maas et al., EMBO J. 9:3447-3452 (1990)).

Hexose kinases are a class of enzymes responsible for the phosphorylation of hexoses, and are classified into two groups. Hexokinase (EC 2.7.1.1) can phosphorylate either glucose or fructose, with different isoforms often unique to different tissues or plant species. Different isoforms can have affinities for different hexoses (Turner and Copeland, Plant Physiol. 68:1123-1127 (1981); Copeland and Turner, In: The Biochemistry of Plants, Vol. 11, Stumpf and Conn (eds.), Academic Press, New York, pp. 107-128 (1987)). Hexokinases include fructokinases (EC 2.7.1.11), which typically have specific affinities for fructose (Doehlert, Plant Physiol. 89:1042-1048 (1989); Renz and Stitt, Planta 190:166-175 (1993). Fructokinases can also be specific in their affinity for nucleotides. The extent to which a fructokinase utilizes UTP may play a physiological role in how efficiently UDP can be recycled for sucrose synthase activity in a particular tissue (Huber and Akazawa, Plant Physiol. 81:1008-1013 (1986); Xu et al., Plant Physiol. 90:635-642 (1989). UDP levels for the sucrose synthase reaction may be maintained, even in the case of an ATP-specific fructokinase, by the enzyme NDP-kinase (EC 2.7.4.6).

NDP-kinase has been reported in several plant tissues (Kirkland and Turner, J. Biochem. 72:716-720 (1959); Bryce and Nelson, Plant Physiol. 63:312-317 (1979); Dancer et al., Plant Physiol. 92:637-641 (1990); Yano et al., Plant Molec. Biol. 23:1087-1090 (1993)). Fructokinase can be substrate inhibited by fructose. In addition, sucrose synthase can be inhibited by fructose (Doehlert, Plant Sci. 52:153-157 (1987); Morell and Copeland, Plant Physiol. 78:140-154 (1985), Ross and Davies, Plant Physiol. 100: 1008-1013 (1992)). Whereas plant tissues where sucrose is catabolized by sucrose synthase predominantly contain fructokinases (Xu et al., Plant Physiol. 90:635-642 (1989); Kursanov et al., Soviet Plant Physiol. 37:507-515 (1990); Ross et al., Plant Physiol. 90:748-756 (1994)), plant tissues where sucrose is catabolized by invertase often contain hexokinases (Nakamura et al., Plant Physiol. 81:215-220 (1991)). Tissues which have both invertase and sucrose synthase activity may contain both hexose kinases (Nakamura et al., Plant Physiol. 81:215-220 (1991)). F6P resulting from hexose kinase activity can be further metabolized in glycolysis or used in resynthesis of sucrose by SPS. G6P resulting from hexose kinase activity can enter the pentose phosphate pathway, via G6P dehydrogenase (EC 1.1.1.49), or be converted to F6P by phosphoglucoisomerase (“PGI”) (EC 5.3.1.9) or G1P by phosphoglucomutase (“PGM”) (EC 5.4.2.2) (Rees, In: Encyclopedia of Plant Physiology Vol. 18, Douce and Day (eds.), Springer Verlag, Berlin, 391-417 (1985); Copeland and Turner, In: The Biochemistry of Plants Vol. 11, Stumpf and Conn (eds.), Academic Press, New York, pp. 107-128 (1987); Foster and Smith, Planta 180:237-244 (1993)).

PGI and PGM are reported to be ubiquitous and reversible with commitments of G6P to either F6P or G1P resulting from fluxes in metabolites further along each pathway, i.e., depending on the cell needs for glycolysis (F6P) or starch biosynthesis (G1P) (Edwards and Rees, Phytochem. 25:2033-2039 (1986); Kursanov et al., Soviet Plant Physiol. 37:507-515 (1990); Tobias et al., Plant Physiol. 99:140-145 (1992)). UDPG formed by sucrose synthase may be utilized directly for cellulose or callose biosynthesis via UDP-glucose dehydrogenase (EC 1.1.1.2) (Robertson et al., Phytochem. 39:21-28 (1995)), can be used for sucrose synthesis by SPS or sucrose synthase, or for glycolysis or starch metabolism dependent on further metabolism by UDP-glucose pyrophosphorylase (EC 2.7.7.9). UDP-glucose phosphorylase has been reported to be a largely reversible enzyme (Kleczkowski, Phytochem. 37:1507-1515 (1994)). Flux through UDP-glucose pyrophosphorylase is reported to be influenced by metabolite levels and utilization of reaction products further along in the pathways (Doehlert et al., Plant Physiol. 86:1013-1019 (1988); Huber and Akazawa, Plant Physiol. 81:1008-1013 (1986); Zrenner et al., Planta 190:247-252 (1993)). The reversibility of PGI, PGM and UDPGPPase has been reported to provide for metabolic variability and networking in metabolism, independent of which initial enzyme cleaved sucrose.

The fate of F6P reportedly plays a role in carbohydrate metabolism. NTP-phosphofructokinase (PFK) (EC 2.7.1.11) (Copeland and Turner, In: The Biochemistry of Plants Vol. 11, Stumpf and Conn (eds.), Academic Press, New York, pp. 107-128 (1987); Dennis and Greyson, Plant Physiol. 69:395-404 (1987); Rees, In: The Biochemistry of Plants Vol. 14, Preiss (ed.), Academic Press, San Diego, pp. 1-33 (1988)) is reported to irreversibly convert F6P to F16BP and is associated with glycolysis. The reverse reaction of F16BP to F6P, associated with gluconeogenesis, is essentially irreversible, and is catalyzed by FBPase (EC 3.1.3.11) (Black et al., Plant Physiol. 69:387-394 (1987). Both reactions may be carried out in a reversible manner by a PPi-dependent fructose-6-phosphate phosphotransferase or PPi-phosphofructokinase (PFP; EC 2.7.1.90) (Black et al., Plant Physiol. 69:387-394 (1987).

PPi-dependent fructose-6-phosphate phosphotransferase or PPi-phosphofructokinase is reported to play a role in the generation of biosynthetic intermediates (Dennis and Greyson, Plant Physiol. 69:395-404 (1987); Tobias et al., Plant Physiol. 99:146-152 (1992)) in addition to the cycling of PPi for UDPGPPase and ultimately UDP for sucrose synthase (Huber and Akazawa, Plant Physiol. 81:1008-1013 (1986); Black et al., Plant Physiol. 69:387-394 (1987); Rees, In: The Biochemistry of Plants Vol. 14, Preiss (ed.), Academic Press, San Diego, pp. 1-33 (1988)).

3. Starch Pathway

Starch is the principal storage carbohydrate of plants. Starch is found in both source tissues, such as leaves, and in sink tissues such as expanding leaves, growing seeds, flowers, roots or tubers, and fruit. Starch is synthesized in leaves during the day from photosynthetically fixed carbon and is mobilized at night. The carbon is transported in the form of sucrose to sink tissues where it may be photoassimilated, further metabolized to fuel cell growth and maintenance, or converted to storage compounds such as, proteins, lipids or starch. Starch anabolism and starch catabolism are central to the balance of carbon distribution and, as a consequence, may occur simultaneously in different tissues of the same plant.

Starch is a polysaccharide composed of glucose units connected by α-(1,4) and α-(1,6) linkages. Starch may be found in plant cells as water insoluble grains or granules. During photosynthesis starch is synthesized and stored in chloroplasts. Starch is also synthesized in roots and storage organs such as tubers, fruits and seeds. In these non-photosynthetic tissues, starch granules are stored in a form of plastids called amyloplasts. The size of the granules varies depending upon the plant species. Starch is composed of amylose and amylopectin, two distinct types of glucose polymers. Amylose is primarily linear chains of α-(1,4)-linked glucose molecules with an average chain length of 1000 glucose molecules. Amylopectin is a highly branched glucan chain consisting of approximately twenty α-(1,4)-linked glucose molecules joined by α-(1,6) linkages to other branches.

Starch can comprise up to 65-75% of the dry weight of cereal grains and up to 80% of mature potato tubers. In these crops starch is the primary energy reserve required for germination. Starch forms a major part of animal diets, particularly of their carbohydrate intake. Starch may also be used in many industrial processes such as paper production, textiles, plastics and adhesives. Starch production in a plant directly correlates with yield. Worldwide, starch producing crops, include but are not limited to wheat, rice, maize and potatoes.

Reviews of starch metabolism include Kruger, In: Plant Metabolism, 2^{nd} edition, Dennis et al. (eds.), Addison Wesley Longman, London, pp. 83-104 (1997); Martin and Smith, Plant Cell 7:971-985 (1995); Preiss, In: Biochemistry of Plants, Vol. 14, Preiss (ed.), Academic Press, San Diego, pp. 181-254 (1988); Preiss, Oxf Surv. Plant Mol. Cell. Biol. 7:59-114 (1991); Smith et al., Plant Physiol. 107:673-677 (1995); Steup, In: Biochemistry of Plants, Vol. 14, Preiss (ed.), Academic Press, San Diego, pp. 255-296 (1988).

The last three reported committed steps of the starch biosynthesis pathway are catalyzed by three enzymes, adenosine 5′-diphosphoglucose pyrophosphorylase (ADP-Glc PPase), starch synthase, and starch branching enzymes. ADP-Glc PPase converts α-glucose-1-phosphate and ATP into ADP-glucose and pyrophosphate. Starch synthase adds the ADP-glucose molecule to the unbranched chain of α-1,4-glucose molecules and releases ADP. Starch branching enzymes take a short chain of α-1,4-glucose molecules and links that chain via an α-1,6-glucose bond.

ADP-Glc PPase (EC 2.7.7.27) is an extensively characterized enzyme of the starch biosynthetic pathway. ADP-Glc PPase catalyzes the conversion of glucose-1-phosphate into ADP-glucose in the presence of ATP. ADP-Glc PPase is a tetramer composed of two large and two small subunits. Maize ADP-Glc PPase has been reported to possess molecular masses of 55 and 60 kDa (Preiss et al., Plant Physiol. 92:881-885 (1990). ADP-Glc PPase has also been reported to be subject to allosteric regulation by the activator 3-phosphoglycerate and by the inhibitor inorganic phosphate (Preiss, Oxf. Surv. Plant Mol. Cell. Biol. 7:59-114 (1991)). The large and small subunits have been reported from a diverse range of plants. ADP-Glc PPase clones, both cDNA and genomic, have been isolated and sequenced (Wasserman et al., Cereal Food World 40:810-817 (1995)). Studies have shown genetic modulation of ADP-Glc PPase levels can impact starch yield. For example, transgenic potato plants transformed with ADP-Glc PPase cDNA in the reverse orientation, have been reported to express low levels of ADP-Glc PPase, resulting in the reduction in starch level in the tuber by 70-75% (Muller-Rober et al., EMBO J. 11:1229-1238 (1992)). Conversely, elevated levels of ADP-Glc PPase have been reported to increase starch yield in transgenic plants (Stark et al., Science 258:287-291 (1992)).

UDP-glucose pyrophosphorylase (EC 2.7.7.9) (UDP-Glc PPase) differs from ADP-Glc PPase, in that UDP-Glc PPase utilizes UTP to convert glucose-1-phosphate to UDP-glucose. UDP-glucose is utilized in plants as the glucosyl donor for the synthesis of various carbohydrates including sucrose, cellulose or α-(1,3) glucans. In bacteria, UDP-glucose is reported to be the primary glucosyl donor for the synthesis of glycogen. Plant starch synthase (EC 2.4.1.21) has been reported to have some affinity for UDP-glucose, and therefore, UDP-glucose may be a substrate for the synthesis of starch. UDP-Glc PPase activity has been isolated from plants (Hondo et al., Plant Cell Physiol. 24:61-69 (1983)). An isolated cDNA clone encoding UDP-Glc PPase has been reported from S. tuberosum (Katsube, J. Biochem. 108:321-326 (1990)). A nucleic acid sequence of a S. tuberosum UDP-Glc PPase has been reported to be more homologous to the nucleic acid sequence of UDP-Glc PPase from Dictyostelium discoideum than to ADP-Glc PPase of Oryza sativa or Escherichia coli. Unlike plant ADP-Glc PPase, S. tuberosum UDP-Glu PPase cDNA does not have a chloroplast-specific transit peptide.

Starch synthase (EC 2.4.1.21) is a glucosyl transferase that transfers glucose from ADP-Glc and catalyzes chain elongation via the formation of α-(1,4)-glucosidic linkages (Preiss, Oxf. Surv. Plant Mol. Cell. Biol. 7:59-114 (1991). Starch synthase activity has been reported to be associated with the starch grain (granule bound starch synthase) as well as with the stroma of the plastid (soluble starch synthase) (MacDonald and Preiss, Plant Physiol. 78:849-852 (1985)). Multiple forms of soluble and granule-bound starch synthase have been identified and characterized in the seeds and leaves of higher plant species.

A 60 kDa granule bound starch synthase I (the waxy protein) is reported to be responsible for the amylose formation (Klosgen et al., Mol. Gen. Genet. 203:237-244 (1986), Shure et al., Cell 35:225-233 (1983)). A waxy mutation yields a phenotype with 100% amylopectin in contrast to wild type maize, which contains about 70% amylopectin and 25-30% amylose. Analysis of proteins associated with the starch granule in waxy maize showed the absence of a major protein of 60 kDa. Furthermore, the starch synthase activity of isolated granules was diminished (Echt and Schwartz, Genetics 99:275-284 (1981)). Additional evidence that granule bound starch synthase I is the waxy gene product which is responsible for amylose biosynthesis was obtained when the expression of the antisense waxy RNA chimeric gene was shown to inhibit granule bound starch synthase synthesis (Visser et al., Mol. Gen. Genet. 225:289-296 (1991)).

Soluble starch synthase is functionally defined as the enzyme recovered in 16,000×g supernatants, or which is released into this supernatant from the granule by gentle or mild agitation. Efforts to purify soluble starch synthases have been complicated by low recoveries of starch synthase polypeptides in soluble extracts and by stability problems. Recently, however, it has been possible to correlate specific polypeptides with activity found in partially purified fractions. The sizes of starch synthase polypeptides recovered from soluble extracts have varied from 77 kDa in pea (Denyer and Smith, Planta 186:609 (1992)), 76 kDa in maize (Mu et al., Plant J. 6:151-159 (1994)), to 55 and 57 kDa in rice (Baba et al., Plant Physiol. 103:565-573 (1993)). Reported studies have demonstrated that soluble starch synthases and starch branching enzymes become entrapped within the starch granule matrix during granule enlargement (Martin and Smith, Plant Cell 7:971-985 (1995); Mu-Forster et al., Plant Physiol. 111:821-829 (1996)). It has been reported that soluble and granule bound forms of these enzymes may not be distinct polypeptides.

The biosynthesis of amylopectin is catalyzed by the combined action of both starch synthases and starch branching enzymes (EC 2.4.1.18). α-(1,6) linkages are introduced into α-(1,4) glucan by transfer of a part of the growing α-(1,4)-polyglucose chain to the hydroxyl group of the number 6 carbon of a glucosyl unit of another chain. This reaction has been reported to be catalyzed by a starch branching enzyme. Similar to starch synthase, multiple isoforms of starch branching enzyme have been identified. Maize has been reported to contain three starch branching enzyme isoforms SBEI, SBEIIa and SBEIIb, which have been cloned (Wasserman et al., Cereal Food World 40:810-817 (1995)). Recent evidence indicates that SBE isoforms IIa and IIb are under separate genetic control (Fisher et al., Plant Physiol. 110:611-619 (1996)). Expression of maize SBEI and II cDNAs in Escherichia coli followed by structural analysis of the resultant α-glucan reveals that SBEII transfers oligosaccharide fragments of shorter chain length than SBEI (Guan et al., Proc. Natl. Acad. Sci. USA 92:964-967 (1995)). SBEI has a reported preference for amylose while SBEII has a reported preference for amylopectin (Guan and Preiss, Plant Physiol. 102:1269-1273 (1993)).

A number of enzymes have been reported to be associated with mobilization of starch from leaves to storage organs or the germination of seeds in which starch is a primary energy source. The actions of these enzymes include two categories. Endoamylases split linkages in random fashion in the interior of the starch molecule. Exoamylases hydrolyze from the nonreducing end of the starch molecule, successively resulting in shortened end-products. Another division can be made according to which linkages the enzymes are capable of hydrolyzing.

α-(1,4) linkages of starch are initially hydrolyzed at random by α-amylase (also referred to as 1,4-α-D-glucan glucanohydrorolase (EC 3.2.1.1.)) to produce mixture of shorter straight-chained and branched oligosaccharides called α-limit dextrans, in addition to maltotriose, maltose and, ultimately, glucose. During cereal seedling development, α-amylase and other enzymes secreted from the aleurone and scutellum hydrolyze starch and other materials stored in the endosperm. α-amylase is an endoamylase which liberates poly- and oligosaccharide chains of varying lengths. Dextrinization of the substrate is accompanied by rapid loss of viscosity of the substrate solution. Commercial applications of α-amylase include the thinning of starch in the liquefaction process of the sugar, alcohol, and brewing industries. α-amylases are also used in desizing of fabrics, in the baking industry, in production of adhesives, pharmaceuticals, and detergents, in sewage treatment, and in animal feed.

α-amylase genes have been reported and characterized in rice (Goldman et al., Plant Sci. 99:75-88 (1994)), barley (Rogers et al., Plant Physiol. 105:151-158 (1994)), maize (Young et al., Plant Physiol. 105:759-760 (1994)) and wheat (Lenton et al., Plant Physiol. 15:261-270 (1994)). The phytohormone, GA3, stimulates expression of certain α-amylase genes and many other genes encoding hydrolytic enzymes. The GA3 signal has the reported function of stimulating source metabolism by increasing the mobilization of the nutrients stored in the endosperm (Thomas and Rodriguez, Plant Physiol. 106:1235-1239 (1994)). Expression of α-amylase gene has been reported to be repressed by sucrose, glucose or fructose. Equimolar concentrations of mannitol do not repress α-amylase gene expression, indicating that the sugar repression of gene expression is not a general osmotic response (Yu et al., J. Biol. Chem. 266:21131-21137 (1991)).

β-Amylase (EC 3.2.1.2) (also known as 1,4-α-D-glucan maltohydrolase) occurs commonly in plants and is an exohydrolase that removes maltose residue from the non-reducing end of α-(1,4) amylose chain. This hydrolysis has not been reported to be able to bypass α-1,6-glucosidic bounds of branched substrate (Marshall, FEBS Lett. 46:14 (1974)). The undegraded part of the substrate is β-limit dextrin. Preferred substrates for this enzyme include long malto-oligosaccharide chains in amylose that are produced first by the partial α-amylolysis of starch. Food and beverage industries employ β-amylase to convert starch into maltose solutions.

Starch phosphorylase (EC 2.4.1.1) (also known as amylophosphorylase, polyphosphorylase or α-(1,4) glucan phosphorylase) catalyzes the degradation of α-(1,4) glucans by removal of a single glucosyl moiety. The reaction catalyzed by starch phosphorylase is reversible and kinetic values favor glucan synthesis. A primary in vivo function has been reported to be α-(1,4) glucan degradation (Steup, In: Biochemistry of Plants, Vol. 14, Preiss (ed.), Academic Press, San Diego, pp. 255-296 (1988)). Phosphorylases are found in all starch containing tissues, including leaves and storage organs. Starch phosphorylases have been classified as type I and type II based upon monomer molecular weight, intracellular location, and glucan specificity. The type II (also known as L) enzyme is the predominant reported form in chloroplasts and has a high specificity for maltodextrins, relative to the type I (also known as H) enzyme which is cytoplasm-specific and has a greater affinity for highly branched α-(1,4) glucans like starch or glycogen (Steup, In: Biochemistry of Plants, Vol. 14, Preiss (ed.), Academic Press, San Diego, 255-296 (1988)). Type I or II two enzymes are immunologically distinct. Genes encoding both type I and type II have been reported and described from a number of plant species including S. tuberosum (Brisson et al., Plant Cell 1:559-566 (1989); Sonnewald et al., Plant Mol. Biol. 27:567-576 (1995)), I. batatas (Lin et al., Plant Physiol. 107:277-278 (1995)) and V. faba (Buchner et al., Planta 199:64-73 (1996)).

The α-(1,6) branches of starch, or other long chain glucans containing α-(1,6) linkages such as pullulan, in addition to α-limit dextrans, can be hydrolyzed by α-dextrin endo-1,6-α-glucosidase (EC 3.2.1.41) (also known as pullulanase, pullulan-6-glucanohydrolase, limit dextrinase, debranching enzyme, amylopectin-6-glucanohydrolase or R-enzyme). The α-(1,6) bonds of the smaller multimeric glucans may also be hydrolyzed by oligo-1,6-glucosidase (EC 3.2.1.10) (also known as sucrose-isomaltase, isomaltase or limit dextrinase).

Enzymes preparations of “limit dextrinase” have been reported and described from Pisum sativum L. (Yellowees, Carbohydrate Res. 83:109-118 (1980)), Sorghum vulgare (Hardie et al., Carbohydrate Res. 50:75-85 (1976)), and Hordeum vulgare (Manners and Rowe, Biochem. J. 110:35P (1968)). In addition a full length cDNA clone of “limit dextrinase” has been cloned from Hordeum vulgare (Genbank accession number AF022725). Oligo-1,6-glucosidase is not reported to be capable of hydrolyzing large chain glucans. α-dextrin endo-1,6-α-glucosidase is reported to be capable of hydrolyzing long-chain glucans and short-chain glucans. Manners and Rowe (Biochem J. 110:35P (1968) report two α-1,6-glucosidase activities, one a hydrolysis of small glycans (dextrins or smaller) and the other a hydrolysis of the outermost α-1,6-glucosidic linkages of large-chain glucans (R-enzyme). Bewley and Black report two α-1,6-glucosidase activities (Bewley and Black, In: Seeds: Physiology and Development and Germination, Plenum Press, New York, N.Y. (1994))

Isoamylase which is also known as glycogen 6-glucanohydrolase, (EC 3.2.1.68) has been reported to hydrolyze α-1,6-glucosidic linkages of amylopectin, glycogen, and various branched dextrins and oligosaccharides. Isoamylase has not been reported to be capable of hydrolyzing all α-1,6-linkages of α-limit dextrins, probably because of a reported low affinity towards the shortened side chains. Pseudomonas amyloderamosa isoamylase has been reported to require a substrate with at least three glucose residues in branched chained (Kainuna et al., Carbohydrate Res. 61:345-357 (1978)). Isoamylases are used to debranch starch in production of glucose and maltose. Genes encoding isoamylase have been reported from P. amyloderamosa SB-15 (Amemura et al., J. Biol. Chem. 263:9271-9275 (1988)). P. amyloderamosa SB-15 isoamylase has a signal peptide of 26 amino acid residues. The P. amyloderamosa SB-15 isoamylase sequence is homologous to α-amylases and CGTases in three reported regions and has significant homology with carboxyl terminus of pullulanase (Amemura et al., J. Biol. Chem. 263:9271-9275 (1988)). It is reported that the carboxy terminal similarities are involved in cleavage of the 1,6-linkages.

Modified isoamylase expression of Flavobacterium sp. isoamylase in the tubers of S. tuberosum was reported by Krohn et al., Mol. Gen. Genet. 254:469-478 (1997). A double gene vector was utilized in which the expression of both a Flavobacterium sp. isoamylase gene, Iam, and an Escherichia coli ADP-Glc PPase mutant isoform, glgC16 (Stark et al., Science 258:287-291 (1992)), was under the control of a tuber tissue-specific promoter and targeted to amyloplasts via a chloroplast transit peptide fusion. Starches of the transgenic potatoes showed a significantly higher percentage of branch chains having a degree of polymerization of 30 and higher, in comparison to wild type control starch.

Glucose-1,6-bisphosphate synthase (EC 2.7.1.106) catalyzes the conversion of 3-phospho-glyceroyl phosphate and glucose-1-phosphate to 3-phospho-glycerate and glucose-1,6-bisphosphate. Glucose-1,6-bisphosphate synthase has been reported to utilize glucose-6-phosphate to form glucose-1,6-bisphosphate. Glucose-1,6-bisphosphate has been implicated in the control of several important carbohydrate metabolic enzymes (Beitner, In: Regulation of Carbohydrate Metabolism (Beitner, R. ed.) Vol. 1, pp. 1-27, CRC Press, Boca Raton, Fla. (1985)) including hexokinase, phosphofructokinase, pyruvate kinase, phosphogluconate dehydrogenase and fructose-1,6-bisphosphatase (Piatti et al., Arch. Biochem. and Biophys. 293:117-121 (1992)). The product of this reaction, glucose-1,6-bisphosphate, rather than the enzyme, has been well characterized in higher animals.

4. Phosphogluconate Pathway

The phosphogluconate pathway (OPPP) (also known as the oxidative pentose phosphate pathway, pentose phosphate shunt, or Warburg-Dickens pathway) is one of the two major pathways in plants by which carbohydrates may be ultimately degraded into CO₂, the other being glycolysis followed by the TCA cycle (Brownleader et al., In: Plant Biochemistry Academic Press, New York, pp. 111-141 (1997)). It has been reported that the OPPP generally accounts for 10-15% of the carbohydrate oxidation in cells (apRees In: The Biochemistry of Plants Vol. 3:1-42 (1980)). It has been reported that the primary purposes of the OPPP is production of NADPH for use in biosynthetic reactions and the production of a ribose-5-phosphate for use in nucleic acid biosynthesis (Turner and Turner, In: Biochemistry of Plants—A Comprehensive Treatise, Vol. 2, pp 279-316, (1980)). The subcellular localization of this pathway has been reported to differ between species, cell type, and plastid type being investigated. For example, reported cellular fractionation experiments in spinach leaf cells showed all enzymes of the phosphogluconate pathway were found in chloroplasts, but that only the first two enzymes of that pathway are present in the cytosol (Schnarrenberger et al., Plant Physiol. 108:609-614 (1995)).

In general, OPPP can be divided into two parts, oxidative (the reactions leading up to ribulose-5-phosphate), and non-oxidative (e.g. Williams, Trends Biochem. Sci. 5:315-320 (1980); apRees, In: Encyclopedia of Plant Physiology, Vol. 18, pp. 391-417, (1985)).

The first reported reaction of OPPP is the conversion of glucose-6-phosphate by glucose-6-phosphate dehydrogenase (G6PDH; EC 1.1.1.49) to 6-phosphogluconolactone. The hydrolysis of 6-phosphogluconolactone to 6-phosphogluconate can occur in a nonenzymatically manner or be catalyzed by a lactonase. This reaction is not at equilibrium and is irreversible (Ashihara and Komamine, Plant Sci. Lett. 2:331-337 (1974); Turner and Turner, In: Biochemistry of Plants—A Comprehensive Treatise, Vol. 2, pp. 279-316 (1980)). The hydrolysis of 6-phosphogluconolactone to 6-phosphogluconate is reported to be a critical regulatory step in the phosphogluconate pathway. The hydrolysis of 6-phosphogluconolactone to 6-phosphogluconate has been reported to respond to the concentration of glucose-6-phosphate as well as the NADPH/NADP+ ratio. Inhibition of the hydrolysis of 6-phosphogluconolactone to 6-phosphogluconate by NADPH is consistent with the function of OPPP to provide NADPH (apRees, In: The Biochemistry of Plants, Vol. 3, pp. 1-42 (1980)). cDNA clones for G6PDH have been isolated from several plants including alfalfa (Fahrendorf et al., Plant Mol. Biol. 28:885-900 (1995)) and potato (Graeve et al., Plant J. 5:353-361 (1994)).

6-phosphogluconate is dehydrogenated to ribulose-5-phosphate, NADPH, and CO₂ in an irreversible reaction catalyzed by 6-phosphogluconate dehydrogenase (6PGDH; EC 1.1.1.44). A cDNA clone for 6PGDH has been isolated from alfalfa (Fahrendorf et al., Plant Mol. Biol. 28:885-900 (1995)). The first two steps of the OPPP are the only reported oxidation reactions in that pathway. Other reactions within OPPP serve to regenerate glucose-6-phosphate, as well as producing intermediates such as ribose-5-phosphate that are utilized in nucleic acid biosynthesis.

Ribulose-5-phosphate may be metabolized in one of two pathways. Ribose-5-phosphate isomerase (EC 5.3.1.6) catalyzes the conversion of ribulose-5-phosphate to ribose-5-phosphate, while ribulose-5-phosphate-3-epimerase (also known as pentose-5-phosphate-3-epimerase; EC 5.1.3.1) catalyzes the conversion of ribulose-5-phosphate to xylulose-5-phosphate. Transketolase (EC 2.2.1.1) catalyzes the conversion of ribulose-5-phosphate and xylulose-5-phosphate into sedheptulose-7-phosphate and 3-phosphoglyceraldehyde. Transaldolase (EC 2.2.1.2) catalyzes the conversion of sedheptulose-7-phosphate and 3-phosphoglyceraldehyde into erythrose-4-phosphate and fructose-6-phosphate.

Erythrose-4-phosphate is a substrate associated with the biosynthesis of lignin (Salisbury and Ross, Plant Physiology, Wadsworth Publishing Company, Belmont, Calif., (1978)), or the production of aromatic amino acids via the shikimate pathway (Schnarrenberger et al., Plant Physiol. 108:609-614 (1995)). Clones for potato transaldolase (Moehs et al., Plant Mol. Biol. 32:447-452 (1996)); spinach transketolase (Flechner et al., Plant Mol. Biol. 32:475-484 (1996)); potato ribulose-5-phosphate-3-epimerase (Teige et al., FEBS Lett. 377:349-352 (1995)); and spinach ribulose-5-phosphate-3-epimerase (Nowitzki et al., Plant Mol. Biol. 29:1279-1291 (1995)) have been reported.

Fructose-6-phosphate may enter glycolysis (apRees, In: The Biochemistry of Plants, Vol. 3, pp. 142 (1980)). Fructose-6-phosphate can also be converted to glucose-6-phosphate via phosphohexose isomerase (EC5.3.1.9). Glucose-6-phosphate can be recycled in the OPPP pathway or be utilized during the synthesis of polysaccharides.

Transketolase (EC2.2.1.1) can catalyze the conversion of erythrose-4-phosphate and xylulose-5-phosphate to fructose 6-phosphate and 3-phosphoglyceraledehyde. Likewise, fructose 6-phosphate and 3-phosphoglyceraledehyde may be used in reactions as described above.

5. Galactomannan Pathway

Galactomannan enzymes are involved in carbohydrate modifications. Galactomannans are reserve polysaccharides composed of (1→4) linked β-D-mannopyranosyl residues having side stubs linked to α-D-galactopyranosyl joined by (1→6) linkages. Galactomannans are widely used in food industry, pharmaceuticals, cosmetics, paper and paint industries. Galactomannans are mainly found in members of the Leguminosae, Anonaceae, Convolvulaceae, Ebenaceae and Palmae and are usually located in the endosperm.

α-mannosidase (EC 3.2.1.24) catalyzes the hydrolysis of terminal, non-reducing α-D-mannose residues in α-D-mannosides. α-mannosidase also hydrolyzes heptopyranosides with the same configuration at C-2, C-3 and C-4 as mannose. α-mannosidase enzyme has been detected in several plant species, including Avena sativa, fenugreek, Hevea latex and soybean (Greve and Orden, Plant. Physiol. 60:478-481 (1977); Beaugiraud et al., Bull. Soc. Chem. Biol. 50:621-631 (1968)). α-mannosidase isoforms have be reported in almond, lupin, Phaseolus vulgaris and soybean (Schwartz et al., Arch. Biochem. Biophys. 137:122-127 (1970); Paus and Christensen, Eur. J. Biochem. 25:308-314 (1972)). It has been reported that α-mannosidase levels increase during seed germination (Meyer and Bourrillon, Biochimie 55:5-10 (1973); Agarwal et al., J. Biol. Chem. 243:103-111 (1968); Neely and Beevers, J. Exp. Bot. 31:299-312 (1980)). It has also been reported that α-mannosidase levels increase during the ripening of some fruits (Ahmed and Labavitch, Plant Physiol. 65:1014-1016 (1980)).

α-mannosidase II (EC 3.2.1.14) is a type II membrane protein, predominantly found in medial Golgi cisternae. α-mannosidase II catalyzes the final hydrolysis step in the asparagine-linked oligosaccharide (N-glycan) maturation pathway. It has been reported that α-mannosidase II is involved in both the biosynthesis and breakdown of N-linked glycans (Daniel et al., Glycobiology 4:551-566 (1994); Moremen et al., Glycobiology 4:113-125 (1994)). It has been reported that lysosomal α-mannosidases are soluble and are involved in N-glycan degradation. Golgi and endoplasmic reticulum α-manosidases have been reported to be involved with the biosynthesis of N-glycans (Misago et al., Proc. Natl. Acad. Sci. (USA) 92:11766-11770 (1995)). It has been reported that human genetic defect in α-mannosidase II causes a congenital dyserythropoietic anemia resulting in clustering membrane proteins and formation of unstable erythrocytes (Chui et al., Cell 90:157-167 (1997)).

Mannan-endo-1,4-β-mannosidase (EC 3.2.1.78) catalyzes the hydrolysis of β-D (1→4) mannopyranosyl linkages of mannans, galactomannans, glucomannans and galacto-glucomannans (Dekker and Richards, Adv. Carbohydr. Chem. Biochem. 32:277-352 (1976)). It has been reported that mannan-endo-1,4-β-mannosidase has been isolated and characterised from bacterial, plants and animal sources including fenugreek, guar-seeds, clover and Konjac (Drekker and Richards, Adv. Carbohydr. Chem. Biochem. 32:277-352 (1976); Ahlgren et al., Acta. Chem. Scand. 21:937-944 (1967); Eriksson and Rzedowski, Arch. Biochem. Biophys. 129:683-688 (1969); Emi et al., Agric. Biol. Chem. 36:991-1001 (1972); McCleary and Matheson, Phytochemistry 14:1187-11949 (1975); Villarroya and Petek, Biochim. Biophys. Acta 438:200-211 (1976); Halmer et al., Planta 130:189-196 (1976); Clermont-Beaugiraud and Percheron, Bull. Soc. Chem. Biol. 50:633-639 (1968); Williams et al., Biochem. J. 161:509-515 (1977); Shimihara et al., Agric. Biol. Chem. 39:301-312 (1975)). A crystallized mannan-endo-1,4-β-mannosidase, isolated from Bacillus subtilis, has been reported (Emi et al., Agric. Biol. Chem. 36:991-1001(1972)). It has also been reported that, in seeds, β-D-mannase activity increases upon germination with a simultaneous decrease of D-mannan (Reid and Meier, Verh. Scweiz. Naturforsch. Ges. 151:68-70 (1971); McCleary and Matheson, Phytochemistry, 14:1187-1194 (1975); Clermont-Beaugiraud and Percheron, Bull Soc. Chem. Biol. 50:633-639 (1968)).

G1 1,4-alpha-D-glucan glucohydrolase (also know as glucoamylase (EC 3.2.1.3)) catalyzes the hydrolysis of terminal 1,4 and 1,6 linked α-D-glucose residues successively from non-reducing ends releasing β-D-glucose. Glucoamylase has industrial applications such as the degradation of starch for the production of glucose and fructose. Glucoamylase is produced by many fungi but only by a few bacteria, like Flavobacterium sp and Halobacterium sodomese have been reported to produce glucoamylase (Taniguchi et al., Agric. Biol. Chem. 50:2423 (1986); Ohba and Ueda, Agric. Biol. Chem. 46:2425 (1982)). Nearly all the fungal glucoamylases are glycoproteins which vary in the number of carbohydrate groups present. It has been reported that glucoamylase exists as three isozymes in Aspergillus awamori var kawachi (Pazur and Kleppe, J. Biol. Chem. 237:1002-1007 (1962)). A cloned glucoamylase from A. awamori glucoamylase has been reported (Erratt and Nasim, J. Bacteriol. 166:484-490 (1986); Yamashita et al., J. Bacteriol. 161:567-573 (1985)). It has been reported that cloned glucosamylase from Saccharomyces diastaticus exists as three unlinked genes, STA1, STA2, and STA3 (Erratt and Nasim, J. Bacteriol. 166:484-490 (1986); Yamashita et al., J. Bacteriol. 161:567-573 (1985); Meaden et al., Gene 34:325-334 (1985); Pretorius et al., Mol. Gen. Genet. 203:36-41 (1986); Pardo et al., Nucleic Acid Res. 14:4701-4718 (1983)). Cloned glucoamylase from Rhizopus oryzae has been reported (Tamaki, Mol. Gen. Genet. 164:205 (1978)). Reports of sequence comparison of the known glucoamylases reveal five homologous segments, one of which does not seem to be essential for the amylolytic activity (Polaina and Wiggs, Curr. Genet. 7:109 (1983). Carbohydrate presence has been reported to be important for the maintenance of the three-dimensional structure of glucoamylase. Glucoamylase has also been reported to be 0-glycosidically linked to serine or threonine residues (Tucker et al., BiotenoL Bioeng. Symp. 14:279 (1984)). Molecular weights of glucoamylase varies from 20,000 to 306,000 (Abe et al., J. Appl. Biochem. 7:235 (1985); Bartoszewicz, Acta Biochem. Pol. 33:17-29 (1986); Erratt and Nasim, J. Bacteriol. 166:484-490 (1986)). It has been reported that heavy metals inhibit glucoamylase (Oten-Gyang et al., Eur. J. Appl. Microbiol. Biotechnol 9:129 (1980)). Reports of chemical modification of Aspergillus niger glucoamylase indicate that tryptophane residues are essential for enzymatic activity (Lineback and Baumann, Carbohydr. Res. 14:341 (1970)).

Glucosamine-fructose-6-P aminotransferase (EC 2.6.1.16) catalyzes the formation of glucosamine-6-phosphate. This reaction is the reported rate limiting step of the hexosamine pathway. Glucosamine-fructose-6-P aminotransferase is also reported to be associated with the regulation of the availability of the precursors for N or O glycosylation of the proteins (Marshall et al., J. Biol. Chem. 266:4706-4712 (1991); Traxinger et al, J. Biol. Chem. 266:10148-10154 (1991)). It has been reported that glucosamine-fructose-6-P aminotransferase is insulin-regulated and may play an important role in insulin resistance in cultured cells (McKnight et al., J. Biol. Chem. 267:25208-25212 (1992)). A cloned human glucosamine-fructose-6-P aminotransferase has been reported. This cloned glucosamine-fructose-6-P aminotransferase has been expressed in Escherichia coli. Expression of a 3.1 KB glucosamine-fructose-6-P aminotransferase cDNA, encoding 681 amino acids, in Escherichia coli resulting in a 77 KD protein has been reported (McKnight et al., J. Biol. Chem. 267:25208-25212 (1992)).

Mannosyl-oligosaccharide alpha-1,2-mannosidase (EC 3.2.1.113) catalyzes the hydrolysis of the terminal 1,2-linked alpha-D-mannose residues in the oligo-mannose oligosaccharide MAN₍₉₎(GlcNAc₍₂₎) (Kornfeld et al., Ann. Rev. Biochem. 54:631-664 (1985)).

6. Raffinose Pathway

The biosynthesis of raffinose saccharides has been studied (Dey, Biochemistry of Storage Carbohydrates in Green Plants, Academic Press, London, pp. 53-129 (1985)). Galactinol synthase initiates the first reported committed step in this pathway. Subsequently, specific galactosyl transferases catalyze the formation of raffinose and its higher homologues (most commonly stachyose) from galactinol and sucrose.

The enzymes directly involved in the synthesis of raffinose oligosaccharides include: galactinol synthase (EC 2.4.1.123), raffinose synthase (EC 2.4.1.82), and stachyose synthase (EC 2.4.1.67).

Galactinol synthase (also referred to as UDP-D-galactose:myo-inositol D-galactosyltransferase) catalyzes the reported initial reaction of synthesizing galactinol from UDP-D-galactose and myo-inositol in the presence of Mn²⁺. This reaction has been reported in a variety of plants (Tanner et al., Plant Physiol. 41:1540-42 (1966); Tanner et al., Eur. J. Biochem. 4:233-239 (1968); Webb, Plant Physiol. 51: suppl. 12 (1973); Pharr et al., Plant Sci. Lett. 23:25-33 (1981)). It has been reported that galactinol synthase controls the flux of reduced carbon into the biosynthesis of the raffinose saccharides (Handley et al., J. Amer. Soc. Hort. Sci. 108:600-605 (1983); Saravitz et al., Plant Physiol. 83:185-189 (1987)). Galactinol synthase has been purified from zucchini and nucleotide sequences have been reported from zucchini and soybean (Kerr et al., U.S. Pat. No. 5,648,210).

Raffinose synthase (Galactinol:sucrose galactosyltransferase) catalyses the second reported step by transferring a D-galactosyl group from galactinol to sucrose in the following reaction: sucrose plus galactinol yields raffinose plus myo-inositol. Lehle et al., (Eur. J. Biochem. 38:103-110 (1973)) report purifying raffinose synthase from V. faba seeds. Raffinose is widely distributed in higher plants (French, Adv. Carbohydrate Chem. 9:149-184 (1954); Kuo et al., J. Agric. Food Chem. 36:32-36 (1988)). It has been reported that the primary role of raffinose is to store and/or transport carbohydrates. Other reported roles include frost hardiness (Kandler et al., The Biochemistry of Plants, Academic Press, New York, Vol. 3, pp. 221-270 (1980)), and seed viability (Ovcharov et al., Fiziol. Rast. 21:969-974 (1974)).

Stachyose synthase (Galactinol:raffinose galactosyltransferase) transfers a D-galactosyl group from galactinol to sucrose yielding stachyose and myo-inositol. Stachyose synthase has been reported from seeds of P. vulgaris (Tanner et al., Plant Physiol. 41:1540-1542 (1966); Tanner et al., Eur. J. Biochem. 4:233-239 (1968)) and V. faba (Tanner et al., Biochem. Biophys. Res. Commun. 29:166-171 (1967)) as well as leaves of C. pepo (Gaudreault et al., Phytochemistry 20:2629-2633 (1981)). Stachyose is one of the major oligosaccharides in plants and is reported to be an important transport carbohydrate.

Plants have the capability of degrading raffinose oligosaccharides through the following enzymes: β-fructofuranosidase (EC 3.2.1.26) and α-galactosidase (EC 3.2.1.22).

α-galactosidase catalyzes the hydrolysis of the 1-6 linkage in the raffinose oligosaccharides. This enzyme has been reported from almost 60 different eukaryotic and prokaryotic sources (Pridham et al., Plant Carbohydrate Biochemistry, Academic Press, London, pp. 83-96 (1974); Itoh et al., J. Biochem. 99:243-250 (1986); Pederson et al., Can. J. Microbiol. 26:978-984 (1980); Duffaud et al., Appl. Environ. Microbiol. 63:169-177 (1997); Davis et al., Biochem. and Mol. Biol. Int. 39:471-485 (1996)). A number of nucleotide sequences have also been published (Davis et al., Biochem. and Mol. Biol. Int. 39:471-485 (1996); Zhu et al., Gene 140:227-231 (1994); den Herder et al., Mol. Gen. Genet. 233:404-410 (1992)).

β-fructofuranosidase (fungal invertase) has been reported to liberate fructose from both sucrose and galactooligosaccharides (Cruz et al., J. Food Sci 46:1196-1200 (1981)). A fungal invertase produces melibiose and manninotriose from raffinose and stachyose, respectively. It has been reported that a combination of fungal invertase and alpha-galactosidase is more efficient in the hydrolysis of galactooligosaccharides in soya meal and canola meal (Slominski, J. Sci. Food Agric. 65:323-330 (1994)).

7. Complex Carbohydrate Synthesis/Degradation Pathways

The term “complex carbohydrate” has been used to distinguish simple carbohydrates from polysaccharides. Simple carbohydrates include the mono, di, tri, and tetra-saccharides and sugar alcohols present in food. Other oligosaccharides containing up to 19 residues can also be considered non-complex. Complex carbohydrates such as starch and cell wall polysaccharides can display a wide range of chemical and physical properties.

The cell wall is the principal structural element of plant form. A plant's cell wall contains a network of cellulose and cross-linking glycans embedded in a gel matrix of pectic substances and is reinforced with structural proteins and aromatic substances (McCann and Roberts, In: Architecture of the Primary Cell Wall, Lloyd (ed.), Academic Press, London, pp. 109-129 (1991); Carpita and Gibeaut, Plant J. 3:1-30 (1993)). Cellulose (β-1,4-glucan) and callose (β-1,3-glucan) have been reported to be synthesized at the plasma membrane of plant cells. Non-cellulosic polysaccharides, the matrix polysaccharides, have been reported to be synthesized in the golgi apparatus, packaged in secretory vesicles, and exported to the surface, where they are integrated with cellulose microfibrils.

Glycosyl transferases located on the plasma membrane of higher plants play a role in the biosynthesis of various cell wall biopolymers. Polysaccharides that have been studied include β-linked glucans cellulose and callose. Cellulose is comprised of β-(1,4) linkages of glucose. Delmer and Amor, (Plant Cell 7:987-1000 (1995)) review the synthesis of this long chain glucan. Cellulase (EC 3.2.1.4), also referred to as endoglucanase and endo-1,4-β-glucanase, catalyzes the endohydrolysis of the β-(1,4) linkages of cellulose and can also hydrolyze the β-(1,4) linkages of more complex glucans containing 1,3-linkages. Among plants, a cDNA encoding cellulase has been reported from Arabidopsis thaliana (Accession number U37702), Glycine max (Accession number U34755), Poplus alba (Accession number D32166), Phaseolus vulgaris (Accession number U34754) and Persea americana (Tucker et al, Eur. J. Biochem. 112:119-124 (1987)). Cellulase has been reported to be active during germination and to degrade cell wall structural carbohydrates to better enable radical emergence. Cellulase may also play a role in protection against pathogen invasion.

Callose has been reported to be synthesized at the plasma membrane when cells are damaged (Delmer, Annu. Rev. Plant Physiol. 38:259-290 (1987)) and at specific stages of development such as pollen tube growth or phragmoplasts of dividing cells. The β-(1,3) linkages of the callose glucan have been reported to be formed by β-(1,3) glucan synthase (EC 2.4.1.34). β-(1,3) glucan synthase is also known as 1,3-β-D-glucan-uridine diphosphate glucosyltransferase (1,3-β-D-glucan-UDP glucosyltransferase), UDP-glucose-1,3-β-D-glucan glucosyltransferase and callose synthetase. β-(1,3) Glucan synthase has been reported to be located in the plasma membrane. Like cellulase, the substrate of β-(1,3) glucan synthase has been reported to be UDP-glucose (Delmer, Annu. Rev. Plant Physiol. 38:259-290 (1987). Hayashi et al., Plant Physiol. 83:1054-1062 (1987)) have reported that micromolar amounts of Ca²⁺ are required for enzyme activity and that such levels may act to endogenously signal cell or membrane damage. Ca²⁺ has been reported to increase the rate of callose formation (Fredrickson and Larson, Biochem. Soc. Trans. 20:210-713 (1992)). The addition of Mg²⁺ has been reported to result in the production of greater amounts of insoluble polymer.

β-1,4-glucosidase (EC 3.2.1.21) has been reported to be found in both prokaryotes and eukaryotes, performing multiple functions in both (Esen, β-glucosidases: Biochemistry and Molecular Biology, ACS Symposium Series, 533, ACS, Washington, D.C. (1993)). In plants, β-glucosidases are involved in the hydrolysis of β-1,4-glucans, such as cellulose and other cell wall polysaccharides, which results in the release of β-D-glucose. The hydrolysis of β-1,4-glucans becomes more pronounced during seed germination. β-(1,3) glucan synthase linkage of callose has been reported to be hydrolyzed by the activity of glucan endo-1,3-β-D-glucosidase (EC 3.2.1.39). Glucan endo-1,3-β-D-glucosidase is also referred to as endo-1,3-β-glucanase, (1,3)-β-D-glucan endohydrolase or laminarinase. Akiyama et al., Carbohydrate Research 297:365-374 (1997)), have reported the activity of a glucan endo-1,3-β-D-glucosidase. Both β-1,4-glucosidase and β-1,3-glucosidase have also been reported to participate in chemical defense against pathogens and the regulation of plant phytohormones such as cytokinin, gibberellin and auxin. Genes encoding β-glucosidase have been reported from a number of plant species including, but not limited to, Hordeum vulgare L. (Accession number L41869, Leah et al., J. Biol. Chem. 270:15789-15797 (1995)), Nicotiana tabacum (Accession number M60403), maize (Accession number U25157) and Avena sativa (Accession number X78433).

Pectin is a major component of the primary cell wall of dicots and has been reported to play a role in cell growth. Pectin is composed of several polymers. The neutral pectins are arabinans, galactans and arabinogalactans. The acidic pectins include rhamnogalacturonan (Darvill et al., In: The Biochemistry of Plants, Stupf and Conn (eds.), Academic Press, New York, Vol. 1, pp. 91-62 (1980)). Rhamnogalacturonic acid consists of chains of α-(1,4) linked galacturonic acid residues interspersed with rhamnose. The carboxyl function of the galacturonosyl residues can be present as a methyl ester, acid or salt.

Pectin methylesterase (EC 3.1.1.11) is also referred to as pectase, pectin demethoxylase, pectin methoxylase and pectinesterase. Pectin methylesterase has been reported to be a ubiquitous enzyme in plants. Pectin methylesterase catalyzes the de-esterification of methoxylated pectins in the cell wall and has been reported to be responsible for chemical modifications of pectin embedded in the plant's primary cell wall matrix. Pectin methylesterase has also been reported to be involved in cell wall growth regeneration the separation of root border cells from the root cap and in the formation of abscission zones and textural changes in ripening fruit (Sexton and Robert, Annu. Rev. Plant Physiol. 33:133-162 (1982); Lamport, In: The Primary Cell Wall: A New Model, Young and Rowell (eds.), John Wiley and Sons, New York, pp. 77-90 (1986); Shea et al., Planta 179:293-308 (1989); Nari et al., Biochem. J. 26:343-350 (1991); Stephenson and Hawes, Plant Physiol. 106:739-745 (1994); Tieman and Handa, Plant Physiol. 106:429-436 (1994)). Multiple isozymes of pectin methylesterase have been reported to be present in different tissues of plants, including tomato (Gaffe et al., Plant Physiol. 105:199-203 (1994)). The clones encoding these isoenzymes of pectin have been isolated (Gaffe et al., Plant Physiol. 105:199-203 (1996)).

Pectinase (EC 3.2.1.15), an endo-acting polygalacturonase, has been isolated from fruits and its expression has been reported to be correlated with the rate of tissue softening. Pectinase catalyzes the digestion of pectin, which results in the release of two rhamnogalacturonan fragments. In tomato (Tucker et al, Eur. J. Biochem. 112:119-124 (1980), pear (Ahmed and Labavitch, Plant Physiol. 65:1014-1016 (1980)) and avocado (Awad and Young, Plant Physiol. 64:306-308 (1979)), pectinase activity in unripe tissue has been reported to be low or absent and increases during ripening. Further correlation has been made between the fruit softening rate and pectinase activity in tomato ripening mutants. Pectinase activity in the never ripe mutant has been reported to be reduced to 10% of its normal level. In the never ripe mutant, it has been reported that the reduced pectinase activity corresponds to a slower rate of softening. The ripening inhibitor mutant (rin) has been reported to have no detectable pectinase activity and does not soften (Tigchelaar et al., Hort. Science, 13:508-513 (1978)). Active pectinase during tomato fruit ripening has a catalytic domain that is necessary for pectin degradation. A second polypeptide in the pectinase complex, a 38-kD “β-subunit”, modifies pH, thermal stability and increases the binding of pectinase to cell walls (Watson et al., Plant Cell 6:1623-1634 (1994)). Plants with antisense of the β-subunit have been reported to show a 60% increase in polyuronide solubilization during ripening. Subsequently, fruit ripening has been reported to result from the degradation of pectin by pectinase. Transgenic plants expressing genetically altered pectinase could have an altered rate of fruit ripening.

α-arabinofuranosidase (EC 3.2.1.55), also known as arabinosidase, hydrolyzes α-L-arabinofuranosidic linkages. α-L-arabinofuranosidase has been reported in lupin seeds at the resting stage and at increased levels during germination. A fraction of α-L-arabinofuranosidase activity was reported to be cell-bound and the rest was reported to be soluble. α-arabinofuranosidase activity has been reported to increase with fruit ripening. After the fruit enlargement stage, cell wall-bound α-L-arabinofuranosidase activity was reported to increase 15-fold with fruit ripening (Tateishi et al., Phytochemistry 42:295-299 (1996)).

It has been reported that in lupin seeds germination causes modification of the chemical structure of the primary cell-wall polysaccharides. α-L-arabinofuranosidase and β-galactosidase have been reported to aid this modification in chemical structure, which occurs in fruit softening, especially in instances where pectin-degrading enzymes have not been detected. α-L-arabinofuranosidase has also been reported to have a role in cancer chemotherapy with antineoplastic compounds (Butschak et al., Arch. Geschwulstforsch. 46:365-375 (1976)). α-L-arabinofuranosidase enzymes with an optimum pH of 6 were reported to be suitable for selective activation of antitumor compounds. β-peltatin α-α-L-arabinofuranoside, for example, was used in combination with α-L-arabinofuranosidase, which caused slow release of the active component.

Chitin has also been referred to as a homopolymer of N-acetylglucosamine. N-acetylglucosamine is also known as (1,4)-2 acetamido-2-deoxy-β-D-glucan. N-acetylglucosamine is a main cell wall component in fungi and yeast. Chitinase (EC 3.2.1.14), an enzyme which degrades chitin, has been reported to exist in microorganisms, plants and in the digestive tracts of animals which feed on chitin-containing organisms. Insects shed their old cuticles, which are made primarily of chitin, during molting for growing and during transformation to more mature stages. Chitin in the old cuticle is degraded to chitooligosaccharides by chitinase. Plant chitinases are induced in response to infection by exogenous plant pathogens or are accumulated in seeds or tubers. Plant chitinases have been reported to play a role in self-defense reactions against fungi and insects that contain chitin in their cell walls and exoskeleton (Koga, Kichin. Kotosan Kenkyu 2:88-89 (1996)).

Chitinase is also known as β-1,4-poly-N-acetyl glucosamidinase, chitodextrinase and poly-β-glucosaminidase. Chitinase hydrolyzes the polymers of N-acetyl-D-glucosamine (Zhnioglu, J. Fac. Sci. 19:63-73 (1996)). Chitinase precursors contain a N-terminal signal peptide and a main catalytic domain. Some chitinase precursors have a chitin-binding domain or C-terminal signal vacuole-directing peptide. Most chitinases have been reported to be inducible by biotic factors such as pathogens and oligosaccharides, or abiotic factors such as ethylene, salicylic acid, salt solutions, ozone and UV light (Punja and Zhang, Can. J. Nematol. 25:526-540 (1993)). The inducibility of chitinase has been reported to be tissue-specific and development-regulated. Chitinase precursors become matured proteins by removal of N-terminal signal peptide, hydroxylation of a proline and removal of C-terminal signal peptide.

Genes encoding chitinase have been reported from various sources of microorganisms and plants (Sueda et al., Kichin Kitosan Kenkyu 1:128 (1995); Nishizawa, Nogyo Seibutsu Shigen Kenkyusho Kenkyu Hokoku 10:73-104 (1995); de A. Gerhardt et al., FEBS Lett. 419:69-75 (1997); Wu et al., Plant Mol. Biol. 33:979-987 (1997); Hudspeth et al., Plant. Mol. Biol. 31:911-916 (1996)). It has been reported that a gene (chiSH1) encoding chitinase was isolated from a bacterium capable of efficiently digesting chitin K. zopfii K12-119. Ikeda et al., (Nippon Shokubutsu Byori Gakkaiho 62:11-16 (1996)), applied viable cells of Escherichia coli transformed with chiSH1 to barley leaves inoculated with the powdery mildew pathogen. Growth of the pathogen was reported to be effectively suppressed by the treatment, indicating the effectiveness of chitinolytic microbes as biocontrol agents.

Some microorganisms have been reported to contain a homopolymer of glucosamine. Glucosamine is also known as (1,4)-2-amino-2-deoxy-β-D-glucan). Unlike the acetylated glycan, chitin, chitosan can be extracted from the cell wall with diluted acid. It has been reported that the crystalline conformation of chitosan is only detectable after it is extracted from the wall with dilute acid. Coating postharvest produce with chitosan has been reported to delay the ripening process and maintain quality attributes of fruit (Arul and Ghaouth, Can. Adv. Chitin Sci. 1:372-380 (1996)). Chitosan coating has been reported to be effective as a fungicide in controlling the decay of strawberry fruit. Chitosan has also been reported to effectively inhibit the decay caused by Botrytis cinerea in tomato and bell pepper. Additionally, chitosan treatment has been reported to stimulate the activities of chitinase, chitosanase and β-1,3-glucanase, which contribute to a plant fungal defense.

Chitosanase (EC 3.2.1.132) hydrolyzes chitosan from its polymerized structure to its oligomer. Chitosanases occur in soil microorganisms and in plants. It has been reported that chitosanase may play a defensive role in plants. It has also been reported that the pharmaceutical industrial may utilize chitosanase for the generation of size-specific chitosan oligomers (Somashekar and Joseph, India Bioresour. Technol. 58:197-237 (1996)).

Trehalose, also know as α-D-glucopyranosyl-α-D-glucopyranoside, is a carbohydrate synthesized by cultured Bradyrhizobium japonicum. Rhizobium have also been reported to accumulate trehalose (Streeter, J. Bacteriol. 164:78-84 (1985)). Trehalose has also been reported in yeast, fungi, bacteria and Actinomycetes. In soybean, trehalose has been reported to be restricted to nodule tissue.

The biosynthesis of trehalose in microorganisms involves, but is not limited to the following enzymes: phosphoglucomutase, UDP-glucose pyrophosphorylase (EC 2.7.7.9), α,αtrehalose-6-phosphate synthase (EC 2.4.1.15), and trehalose phosphatase. In the cytosolic phase of the cell, α-D-glucose-6-phosphate is converted into α-D-glucose-1-phosphate through the action of phosphoglucomutase (EC 5.4.2.2). α-D-glucose-1-phosphate is then utilized by UDP-glucose pyrophosphorylase (EC 2.7.7.9) to produce UDP-glucose. UDP-glucose is then converted into α,α-trehalose-6-phosphate in the presence of α-D-glucose-6-phosphate by α,α-trehalose-6-phosphate synthase (EC 2.4.1.15). Trehalose phosphatase (EC 3.1.3.12) then incorporates α,α-trehalose-6-phosphate into α,α-trehalose, with the generation of free orthophosphate (Salminen and Streeter, Plant Physiol. 81:538-541 (1986)). Enzyme activity of phosphoglucomutase, α,α-trehalose-6-phosphate synthase and trehalose is dependent on the presence of Mg²⁺.

Trehalose hydrolysis is catalyzed by α,α-trehalase (EC 3.2.1.28) in the cytosolic phase of the cell to generate two molecules of D-glucose. α,α-trehalase has been reported to be present in microorganisms as well as in some higher plants (Salminen and Streeter, Plant Physiol. 81:538-541 (1986)). In Saccharomyces cerevisias, three trehalases have been reported. The first reported trehalase is the cytosolic neutral trehalase, which is encoded by the NTH1 gene and is regulated by the cAMP-dependent phosphorylation process, available nutrients and temperature. The second trehalase is the vacuolar acid trehalase, which is encoded by the ATH1 gene and is regulated by the availability of nutrients. The third trehalase is a putative trehalase, Nth1p, which is encoded by the NTH2 gene, a homologue of the NTH1 gene, and is regulated by the availability of nutrients and temperature. Neutral trehalase has been reported to be responsible for the intracellular hydrolysis of trehalose. Acid trehalase has been reported to be responsible for utilization of extracellular trehalose. The NTH1 and NTH2 gene are reported to be required for the recovery of cells after heat shock at 50° C. Other stressors, such as toxic chemicals, have also been reported to induce the expression of the NTH1 and NTH2 genes (Solomon and Helmut, Prog. Nucleic Acid Res. Mol. Biol. 58:197-237 (1998)).

Extracellular α,α-trehalose can be transferred into the cytosolic phase of the cell by the plasma membrane by phosphoenolpyruvate (PEP)-dependent phosphorylation. Extracellular α,α-trehalose can then be converted into α,α-trehalose-6-phosphate. α,α-trehalose-6-phosphate can be further hydrolyzed into D-glucose-6-phosphate through the action of α,α-phosphotrehalase (EC 3.2.1.93). In Bacillus popilliae, phosphotrehalase the range of pH 6.5 to pH 7.0 was reported to be optimum. The optimum Kₘ for trehalose-6-phosphate was reported to be 1.8 mM. It has been reported that a mutant missing phosphotrehalase failed to grow on trehalose and grew normally on other sugars. The mutant lacking phosphotrehalase has been reported to accumulate trehalose-14C as trehalose-14C-6-phosphate. Phosphorylation of trehalose was reported to be at least two times faster with PEP than with ATP, and the phosphorylation activity was associated primarily with the particulate fraction. Subsequently, it has been reported that trehalose is transported into the cell as trehalose-6-phosphate by a PEP:sugar phosphotransferase system (Bhumiratana et al., J. Bacteriol. 119:484-493 (1974)).

The trehalose biosynthetic pathway is associated with the paramylon degradation pathway. Paramylon is composed of 1,3-β-D-glucosyl linkage. Paramylon can be degraded into 3-β-D-glucosylglucose, also known as laminaribiose, by α,α-trehalose-phosphorylase (EC 2.4.1.64). α,α-trehalose-phosphorylase has also been reported to generate α-D-glucose-1-phosphase that can contribute to trehalose biosynthesis. α,α-trehalose-phosphorylase then further converts laminaribiose into D-glucose. D-glucose is then incorporated into β-D-glucose-6-phosphate by β-phosphoglucomutase (EC 5.4.2.6).

Aldose reductase (EC 1.1.1.21) is a member of the NADPH-dependent aldoketoreductase superfamily. Aldose reductase catalyzes the NADPH-dependent reduction of dome aldehydes to their corresponding sugar alcohols. Aldose reductase has been reported to exist in mammals, birds, plants, fungi and bacteria (Markus et al., Biochem. Med. 29:31-45 (1983); Wirth and Wermuth, Prog. Clin. Biol. Res. 174:231-239 (1985); Davidson et al., Comp. Biochem. Physiol. 60:309-315 (1978); Carper et al., FEBS Lett. 220:209-213 (1987); Bohren et al., J. Biol. Chem. 264:9547-9551 (1989); Schade et al., J. Biol. Chem. 265:3628-3635 (1990); Bartels et al., EMBO. J. 10: 1037-1043 (1991)). Aldose reductase activity has been reported to result in an increase in sorbitol and galactitol in the cells of some tissues (Kador and Kinoshita, Am. J. Med. 79:8-12 (1985)). Accumulation of sugar alcohols has been reported to cause osmotic cataracts in eye lens.

D-xylulose reductase (EC 1.1.1.9) catalyzes the reversible conversion of D-xylose to D-xylitol. D-xylulose reductase has been reported to participate in the synthesis of xylitol which is an acarcinogenic, non-caloric sweetener (Kulbe et al., Prog. Biotechnol. 7:565-572 (1992)). D-xylulose reductase has been purified from yeast (Ditzelmuller, Appl. Microbiol. Biotechnol. 22:297-299 (1985); Verduyn et al., Biochem. J. 226:297-299 (1985)). Studies on xylulose reductase from Pichia stipitis have reported histidine and cysteine residues that are associated with the binding of cofactors.

Glycosyltransferase catalyzes the synthesis of carbohydrate moities of glycoproteins, glycolipids and proteoglycans. Glycosyltransferases have been reported to exist on the membranes of the endoplasmic reticulum and golgi apparatus. Glycosyltransferases transfer sugar groups from an activated donor, usually a nucleotide sugar, to a growing carbohydrate group. The structure of the sugar chains produced by a cell depends on the specificity of the glycosyltranserases for their acceptors and donors. It has been reported that more than 100 glycosyltransferases are required for the synthesis of known carbohydrate structures on glycolipids or glycoproteins. (Sadler, In: Biology of Carbohydrates, Ginsburg, and Robbins (eds), John Wiley and Sons, New York, Vol. 2, pp 87-131 (1984); Beyer et al., Adv. Enzymol. Relat. Areas Mol. Biol. 52:23-175 (1981)). Glycosyltransferases are grouped according to the type of sugar they transfer. Galactosyltransferases and sialyltransferases are examples of this type of grouping. Glycosyltransferases have been isolated and purified to homogeneity from mammalian sources (Sadler, Biology of Carbohydrates, Ginsburg, and Robbins (eds), John Wiley and Sons, New York, Vol. 2, pp 87-131 (1984); Beyer et al., Adv. Enzymol. Relat. Areas Mol. Biol. 52:23-175 (1981); Blanken et al., J. Biol. Chem. 260:12927-12934 (1985); Elices et al., J. Biol. Chem. 261:6064-6072 (1986); Prieels et al., J. Biol. Chem. 256:10456-10463 (1981)). Isolated glycosyltransferases include galactosyltransferase (Shaper et al, J. Biol. Chem. 263:10420-10428 (1988); Masri et al., Biochem. Biophys. Res. Commun. 157:657-663 (1988); Joziasse et al., J. Biol. Chem. 264:14290-14297 (1989)), sialyltransferase, fucosyltransferase (Kornfeld and Kornfeld, Annu. Rev. Biochem. 54:631-664 (1985)) and N-acetylgalactosaminyltransferase (Sadler, Biology of Carbohydrates 2:87-131 (1984)). Sequence comparison studies have revealed little sequence homology between these proteins (Kornfeld and Kornfeld, Annu. Rev. Biochem. 54:631-664 (1985); Sadler, Biology of Carbohydrates 2:87-131 (1984)). All the glycosyltransferases, however, have been reported to have a similar short NH₂-terminal cytoplasmic tail, a 16-20 anchor domain and a stem region which attaches a large COOH terminal catalytic domain to the anchor domain (Paulson et al., Biochem. Soc. Trans. 15:618-620 (1987)). It has been reported that glycosyltransferases are present in the golgi apparatus (Roth, Biochem. Biophys. Acta 906:405-436 (1987)).

Subcompartmentalization of glycosyltransferases within the golgi has been reported. N-acetylglucosamyltransferase I has been reported to be localized in the medial cisternae and GlcNAc β 1,4-galactosyltrasnerase, Gal α-2,6-sialytransferase has been reported to be present in the trans citernae (Berger and Hesford, Proc. Natl. Acad. Sci. (U.S.A.) 82:4736-4739 (1985); Bergeron et al., Biochem. Biophys. Acta 821:393-403 (1985); Duncan and Kornfeld, J. Cell. Biol. 106:617-628 (1988)).

Glycosyltransferases have been reported to be differentially expressed during differentiation and oncognic transformation (Rademacher et al., Glycobiology Annu. Rev. Biochem. 57:785-838 (1988)). Glycosyltransferase expression has been reported to be regulated at the transcription level. Paulson has reported that the level of Gal α-2,6-ST increases 4-5 fold in the liver after inflammation (Paulson et al., J. Biol. Chem. 264:10931-10934 (1989)). Wang et al., (J. Biol. Chem. 264:1854-1859 (1989)), have reported an increase in sialyltransferase levels during liver inflammation. It has been reported that changes in glycosyltransferase expression also produce changes in glycolipid or glycoprotein glycosylation (Coleman et al., J. Biol. Chem. 250:55-60 (1975); Nakaishi et al., Biochem. Biophys. Res. Commun. 150:760-765 (1988); Matsuura et al., J. Biol. Chem. 264:10472-10476 (1989)).

L-arabinose-isomerase (EC 5.3.1.4) catalyzes isomerization or intramolecular oxidoreduction. L-arabinose-isomerase catalyzes the isomerization of L-arabinose to L-ribulose. L-arabinose-isomerase has been purified from various sources including Salmonella typhimurium, Lactobacillus gayonii, L. plantarum, Escherechia coli and Streptomyces sp. (Lin et al., Gene 34:123-128 (1985)). A gene encoding L-arabinose-isomerase has been reported (Lin et al., Gene 34:123-128 (1985); Wilcox et al., J. Biol. Chem. 248:2946-2952 (1974)).

Pullulanase hydrolyzes 1,6-linkages of pullulan and other branched oligosaccharides. Pullulanase has been reported to have been found in microbes, including Klebsiella pneumoniae. A Klebsiella pneumoniae ATCC 15050 pullulanase gene has been reported to have been isolated from Escherichia coli (Michaelis et al., J. Bacteriology 164:633-638 (1985)). It has been reported that genes for two additional pullulanase genes have also been isolated. One pullulanase gene from Thermoanaerobacterium brockii has been cloned into E. coli and B. subtilis (Coleman et al., J. Bacteriol. 169:4302-4307 (1987)) and another pullulanase gene from Thermus sp. AMD-33 has been cloned into E. coli (Sashibara et al., Microbiol. Lett. 49:385-390 (1988)). Pullulanase is a cell-bound enzyme intracellular and extracellular enzyme. It has been reported that the intracellular and the extracellular forms have similar properties (Walenfels et al., Biochem. Biophys. Res. Commun. 22:254-261 (1960)). Clostridium thermohydrosulfuricum, T. brockii and Thermus sp. AMD-33 have been reported to produce thermostable pullulanase (Hyun and Zeikus, Appl. Environ. Microbiol. 49:1168-1173 (1985); Coleman et al., J. Bacteriol. 169:4302-4307 (1987); Sashibara, FEMS Microbiol. Lett. 49:385-390 (1988)). The molecular weight of pullulanase has been reported to vary from 80,000-kDa to 145,000-kDa. It has been reported that pullulanase exists as a monomer (Katsuragi et al., J. Bacteriol. 169:2301-2306 (1987)). Heavy metal ions and cyclodextrins have been reported to inhibit pullulanases. Pullulanase purified from Bacillus sp. No. 202-1 was not reported to be inhibited by either PCMB or EDTA (Nakamura et al., Biochem. Biophys. Acta 397:188-193 (1975)). α-galactosidase (EC 3.2.1.22) catalyzes the hydrolysis of terminal non-reducing α-D-galactose residues in α-D-galactosides, including galactose oligosaccharides, galactomannans and galactolipids. Under certain conditions, α-galactosidase has been reported to catalyze de novo synthesis of oligosaccharides (Dey et al., Advan. Enzymol. Relat. Areas. Mol. Biol. 36:91-130 (1972); Pridham and Dey, In: Plant Carbohydrate Biochemistry, Pridham (ed.), Academic Press, London, p 83 (1974)). α-galactosidases have been reported from plants (Dea and Morrison, Adv. Carbohydra. Chem. Biochem. 31:241-312 (1975); Bailey, In: Chemotaxonomy of the Leguminoseae, Harborne et al., Academic Press, London, p. 503 (1971); Courtois, An. Real. Acad. Farm 34:3-32 (1968); McCleary and Matheson, Phytochemistry 13:1747-1757 (1974); Courtois and Percheron, In: Mem. Soc. Bot. Fr., pp 29-39 (1965)), animals and microbes (Dey et al., Advan. Enzymol. Relat. Areas. Mol. Biol. 36:91-130 (1972)). It has been reported that in plants one of the functions of α-galactosidases is to cleave α-D-galactosyl groups from α-D-galactose-containing oligo- and polysaccharides. The degradation product in this reaction serves as an energy source. It has further been reported that α-galactosidase is involved in galactolipid metabolism and chloroplast-membrane function (Bamberger and Park, Plant Physiol. 41:1591-1600 (1970)). α-galactosidase specificities are reported to vary widely. α-galactosidase from Vicia sativa and Mortierella vinacea are reported to hydrolyze small molecular weight substrates but is not reported to act on larger substrates (Petek et al., Eur. J. Bioche., 8:395-402 (1969)). α-galactosidase from coffee bean and Phaseolus vulgaris, however, is reported to hydrolyze both types of substrates (Courtois and Petek, Methods. Enzymol. 28:565-571 (1966); Agarwal and Bahl, J. Biol. Chem. 243:103-111 (1968)). α-galactosidase activity has been reported to increase during the germination of seeds in certain species, including fenugreek (Sioufi et al., Phytochemistry 9:991-999 (1970)), guar, soybean (McCleary and Matheson, Phytochemistry 13:1747-1757 (1974), cotton, and coffee. In many of these plants, it has been reported that there is a concomitant depletion of α-galactosidic reserve carbohydrate. The presence of α-galactosidase isozymes has been reported in the germinated seeds of carob, guar, lucerne and soybean (McCleary and Matheson, Phytochemistry 13:1747-1757 (1974). The presence of two α-galactosidases has been reported in Vicia faba (Dey and Pridham, Advan. Enzymol. Relat. Areas. Mol. Biol. 36:91-130 (1972)).

β-D-galactosidase (EC 3.2.1.23) catalyzes the hydrolysis of β-D-galactoside. β-galactosidase has been reported in many plant species including barley, maize and wheat (Dey, Phytochemistry 16:323-325 (1977); Lee and Ronalds, J. Sci. Food Agric. 23:199-202 (1972)). Isozymes of β-D-galactosidase have been reported to have been found in several plant species (Schwartz et al., Arch. Biochem. Biophys. 137: 122-127 (1970)). β-galactosidase activity has been reported to increase in germinating seeds. Levels of β-D-galactosidase have been reported to increase with the ripening of fruit (Kupferman and Loeschen, J. Am. Chem. Hortic. Sci. 105:452-454 (1980)).

The α(1,4) glucose disaccharide, maltose, can be degraded by maltose phosphorylase (EC 2.4.1.8). The degradation of maltose phosphorylase releases D-glucose and β-D-glucose-1-phosphate. Maltose can be synthesized by the reversal of the maltose degradation reaction. Maltose phosphorylase has been characterized from several bacterial species. Huwel et al., (Enzyme and Microbial Technology 21:413-420 (1997)), have reported a maltose phosphorylase from Lactobacillus brevis. Sucrose α-glucosidase (EC 3.2.1.48), also known as sucrase-isomaltase, sucrose α-glucohydrolase and sucrase, catalyzes the degradation of sucrose to glucose and fructose. Sucrose α-glucosidase is also able to utilize maltose as a substrate, resulting in the release of two molecules of α-D-glucose. The gene encoding sucrose α-glucosidase has been reported to have been isolated from human (Green et al, Gene 57: 101-110 (1987)) and rat (Broyart et al., Biochim. Biophys. Acta, 1087:61-67 (1990)) intestines. Sucrose α-glucosidase has been reported to exhibit a similar enzyme activity in germinating seedlings.

The 3,6-dideoxyhexoses are involved in the serological specificity of several immunologically active polysaccharides. The 3,6-dideoxyhexoses are present primarily as a lipopolysaccharide component of the cell wall of the gram-negative bacteria in which they constitute the nonreducing terminal groups of the O-antigen repeating units. Biosynthesis of 3,6-dideoxyhexoses starts with an internal oxidation-reduction step mediated by an NAD⁺-dependent oxidoreductase. NAD⁺-dependent oxidoreductase catalyzes the transformation of a nucleotidyl diphosphohexose to the corresponding 4-keto-6-deoxyhexose derivative. The 4-keto-6-deoxyhexose derivative can be further catalyzed by a dehydrate and a reductase resulting in 3,6-dideoxyhexose as the final product (Glaser and Zarkowsky, Enzymes, 3^{rd} Vol. 5, pp. 465-480 (1971)).

One of the major reported oxidoreductases in this category is cytidine diphosphate-glucose oxidoreductase (CDP-glucose oxidoreductase). CDP-glucose oxidoreductase (EC 4.2.1.45), also known as CDP-glucose 4,6-dehydratase, has been reported from Yersinia pseudotuberculosis (He et al., Biochem. 35:4721-4731 (1996); Thorson et al., J. Bacteriol. 176:5483-5493 (1994)). CDP-glucose 4,6-dehydratase from Yersinia pseudotuberculosis is an NAD⁺-dependent enzyme which catalyzes the conversion of CDP-glucose to CDP4-keto-6-deoxy-D-glucose. Although CDP-glucose 4,6-dehydratase (EC 4.2.1.45) is a member of the class of enzymes which utilizes tightly bound NAD⁺ as a catalytic prosthetic group, CDP-glucose 4,6-dehydratase (EC 4.2.1.45) is unique in that it requires NAD⁺ for its activity. A gene coding for this enzyme has been reported to have been isolated, sequenced and expressed in Escherichia coli at a level of 5% of the total soluble protein. Comparison of the NAD⁺-binding characteristics of recombinant CDP-glucose, in the absence and presence of substrate, and dehydroquinate synthase, another member of the class, reveals a 2700-fold lower NAD⁺ affinity for the CDP-glucose 4,6-dehydratase. Primary structure correlation of these enzymes indicates differences in the ADP-binding bab fold between CDP-glucose 4,6-dehydratase (EC 4.2.1.45) and dehydroquinate synthase. From this comparison, a potentially new NAD⁺-binding motif has been reported. The reported NAD⁺-binding motif intimates that the weaker binding displayed by a Yersina pseudotuberculosis enzyme which has been reported may be the result of a decrease in the α-helix dipole, an important cofactor for binding, or an increase in protein-cofactor steric interaction.

Malto-oligosaccharides, also known as linear (1,4)-linked α-D-glucopyranosyl-oligosaccharides, can be used as food additives such as sweeteners, gelling agents, viscosity modifiers, fermentation feedstocks, synthons for pharmaceuticals, and experimental substrates for amylases. Malto-oligosaccharides have been produced from the enzymatic digestion of starch by malto-oligosaccharide-forming amylase. In addition, malto-oligosaccharides can also be produced from cyclomaltoheptose (β-cyclodextrin) and cyclomaltooctaose (r-cyclodextrin) using cyclomaltodextrinase (EC 3.2.1.54) (Uchida et al., Carbo. Res. 287:271-274 (1996)).

Cyclomaltodextrinase (EC 3.2.1.54) is also known as cyclodextriase, and cyclodextrin-hydrolyase. Cyclomaltodextrinase has cyclodextrin-hydrolyzing activity and coupling activity. The cyclodextrin-hydrolyzing activity involves the decycling of cyclodextrins. Cyclomaltodextrinase coupling activity involves the transfer of D-glucose to cyclodextrins with a decycling reaction. Optimal substrates of cyclomaltodextrinase include α-, β—, and gamma-cyclodextrins and linear maltooligosaccharides, with maltose as a final product. Cyclomaltodextrinase converts pullulan to panose. Cyclomaltodextrinase can also hydrolyze soluble starch, amylose, and amylopectin, although it cannot hydrolyze glycogen. Cyclomaltodextrinase has been reported from microorganisms, in the bacillus family (Galvin et al., Appl. Microbiol. Biotechnol. 42:46-50 (1994); Bender, Appl. Microbiol. Biotechnol. 43:838-843 (1995); Yang et al., Ann. N.Y. Acad. Sci. 799:425-428 (1996)); Zhong et al., Appl. Biochem. Biotechnol. 59:63-75 (1996)). In Bacillus stearothermophilus, the Kₘ and Vₘₐₓ for α-, β-, and gamma-cyclodextrins were reported to be 1.79 mg/mL and 2.50 mg/mL and 336 mmol/mg/min, 185 mmol/mg/min and 208 mmol/mg/min, respectively. It has been reported that due to the effect of some protein modification reagents on the activity of cyclomaltodextrinase tryptophan, histidine residues may be located at the active site (Zhong et al., Appl. Biochem. Biotechnol. 59:63-75 (1996)).

Cyclomaltodextrinase is involved in the production of maltodextrins and maltooligosaccharides. Genes encoding cyclomaltodextrinase have reported from Bacillus sphaericus (Oguma et al., Appl. Microbiol. Biotechnol. 39:197-203 (1993)), Clostridium thermohydrosulfuricum (Podkovyrov and Zeikus, J. Bacteriol. 174:5400-5405 (1992)), and Bacillus subtilis (Krohn and Lindsay, Curr. Microbiol. 26:217-222 (1993)). A cyclodextrinase gene of Bacillus sphaericus has been cloned and expressed in Escherichia coli and has been manufactured for use in the preparation of maltooligosaccharides and other oligosaccharides (Oguma et al., Ger. Offen. p. 10 (1993)).

Glycogen is produced for use as a deposit of reserve carbohydrate by many organisms. Glycogen is a counterpart of starch in mammalian and bacterial system. The carbohydrate polymer is highly branched with about 90% of its units existing as (1,4)-α-glucosidic linkages and the remainder as (1,6)-α-linkages.

Glycogen synthase (EC 2.4.1.21), also known as glycogen glucosyltransferase, catalyzes the linear elongation of glycogen. In bacterial systems, glycogen synthase uses ADP-glucose as its substrate and it is known as ADP-glucose glycogen glucosyltransferase. In mammalian systems, glycogen synthase uses UDP-glucose as its substrate. In mammalian systems, glycogen synthase is known as UDP-glucose glycogen glucosyltransferase. Glycogen synthase is thought to serve the same function in bacterial and mammalian systems as does starch synthase in higher plants. Glycogen synthase and each of its forms were purified from various sources, such as Escherichia coli (Fox et al., Biochem. 15:849-856 (1976)), Saccharomyces cerevisiae (Huang and Cabib, J. Biol. Chem. 249:3851-3857 (1974); Huang et al., J. Biol. Chem. 249:3858-3861 (1974)) and rabbit muscle (Soderling et al., J. Biol. Chem. 58:197-237 (1970); Brown and Larner, Biochim. Biophys. Acta, 242:69-80 (1971)). Yeast and mammalian glycogen synthase exists in D and I forms. The D form is essentially inactive without glucose-6-phosphate, while the I form reaches nearly full activity without it. The I & D forms of glycogen synthase are interconvertible by phosphorylation and dephosphorylation when catalyzed by a synthase kinase and a synthase phosphatase (Lamer and Villar-Palasi, Curr. Top. Cell Regul. 3:196-236 (1971)). A structural gene for glycogen synthase, glgA, has been isolated from both bacterial and mammalian systems (Okita et al., J. Biol. Chem., 256:5961-5964 (1981); Browner et al., Proc. Natl. Acad. Sci. U.S.A. 86:1443-1447 (1989); Bai et al, J. Biol. Chem. 265:7843-7848 (1990)). Due to the differences in substrate, as well as in regulatory mechanisms, bacterial and mammalian glycogen synthase are not homologous and have been reported to be different molecular entities all together.

In addition to glycogen synthase, the synthesis of bacterial glycogen also requires ADP-D-glucose pyrophosphorylase and glycogen branching enzyme, which are encoded by glgC and glgB, respectively. It has been reported that the major form of ADP-D-glucose pyrophosphorylase in maize endosperm is extra-plastidial (Denyer et al., Plant Physiol. 112:779-785 (1996)). glgC and glgB genes are found as a cluster on the Escherichia coli genome. The order of the genes is reported to be asd-glgB-glgC-glgA, in which asd codes for aspartate semialdehyde dehydrogenase (Okita et al., J. Biol. Chem., 256:5961-5964 (1981)). It has been reported, through the use of photoaffinity labeling using substrate analogues, that the active binding site of glycogen synthase exhibited a conserved region Lys-X-Gly-Gly (Tagaya et al., J. Biol. Chem. 260:6670-6676 (1985)).

Glucose-1-phosphate cytidylyltransferase (EC 2.7.7.33), also known as CDP-glucose pyrophosphorylase, catalyzes the formation of cytidine 5′-diphosphate-glucose from cytosine triphosphate and glucose-1-phosphate. Glucose-1-phosphate cytidylyltransferase is in the α-D-glucose-1-phosphate nucleotidyltransferase class, which includes ADP-D-glucose pyrophosphorylase (EC 2.7.7.27) and UDP-D-glucose pyrophosphorylase (EC 2.7.7.9). Cytidine 5′-diphosphate-glucose is a precursor for some 3,6-dideoxyhexoses found in lipopolysaccharides of gram-negative bacteria (Thorson et al., J. Bacteriol. 176:5483-5493 (1994)). These 3,6-dideoxyhexoses have been reported to be involved in the organisms immunological determination. Cytidine 5′-diphosphate-glucose was also reported to inhibit the self-glycosylating protein, glycogenin, which is reported to be responsible for early steps of glycogen biosynthesis in higher animals (Manzella et al., Arch. Biochem. Biophys. 320:361-368 (1995)). Glucose-1-phosphate cytidylyltransferase has been reported from Salmonella partyphi type A and Azptobacter vinelandii. A gene encoding glucose-1-phosphate cytidylyltransferase has been reported from Yersinia pseudotuberculosis (Thorson et al., J. Bacteriol. 176:5483-5493 (1994)) and from Salmonella enterica (Lindqvist et al., J. Biol. Chem. 269:122-126 (1994)).

CDP-4-dehydro-6-deoxyglucose reductase (EC 1.17.1.1), also known as CDP-4-keto-6-deoxyglucose reductase, participates in the reversible reaction involving the interconversion of nucleotidyl-hexose CDP-4-dehydro-3,6-dideoxy-D-glucose and CDP4-dehydro-3,6-deoxy-D-glucose. CDP4-dehydro-6-deoxyglucose reductase has been reported to be involved in the formation of several antigenic polysaccharide chains of the lipopolysaccharides of the cell envelope of some gram-negative bacteria such as Salmonella enterica (Lindqvist et al., J. Biol. Chem. 269:122-126 (1994)). Polysaccharides containing hexoses such as abequose, ascarylose, paratose and tyvelose are derived from cytidine 5′-diphosphate-glucose (Thorson et al., J. Bacteriol. 176:5483-5493 (1994)).

Glucose-1,6-bisphosphate synthase (EC 2.7.1.106) catalyzes the conversion of 3-phospho-D-glyceroyl phosphate and D-glucose-1-phosphate to 3-phospho-D-glycerate and D-glucose-1,6-bisphosphate. Glucose-1,6-bisphosphate synthase can also use D-glucose-6-phosphate to form D-glucose-1,6-bisphosphate. D-glucose-1,6-bisphosphate has been reported to be associated with the control of several carbohydrate metabolic enzymes including hexokinase, phosphofructokinase, pyruvate kinase, phosphogluconate dehydrogenase and fructose-1,6-bisphosphatase (Beitner, Regul. Carbohydr. Metabol. 1:1-27 (1985)). D-glucose-1,6-bisphosphate has been characterized in higher animals. The metabolism of plant starch and bacterial glycogen has been reported to be similar in the regulation mechanism of (1,4)-α-glucan synthesis.

α-Mannosidase (EC 3.2.1.24), is a carbohydrate-digesting enzyme. α-Mannosidase is associated with early N-linked oligosaccharide processing which removes α-1,2-linked mannose residues from glycoprotein and oligosaccharide substrates. α-1,2-mannosidase activity has been reported in the endoplasmic reticulum and golgi complex of mammalian cells. α-Mannosidase from the endoplasmic reticulum and golgi complex of mammalian cells have been purified and are reported to share biochemical characteristics (Moremen and Herscovics, Guideb. Secretory Pathway, Rothblatt et al. (eds.), Oxford University Press, Oxford, UK, pp. 103-104, (1994)). α-1,2-mannosidases have been reported to be inhibited by 1-deoxymannojirimycin (dMNJ). Distinctions among α-1,2-mannosidase have been made by differences in their specificities to oligosaccharide substrates, their intracellular location and their cation requirements. Two endoplasmic reticulum α-mannosidases have been reported to have catabolic activity. It has been reported that an endoplasmic reticulum/cytosolic mannosidase is involved in the degradation of dolichol intermediates that are not needed for protein glycosylation. It has also been reported that the soluble form of Man9-mannosidase is responsible for the degradation of glycans on defective or malfolded proteins that are specifically retained and broken down in the endoplasmic reticulum. It has further been reported that, based on inhibitor studies with pyranose and furanose analogs, α-mannosidases may be divided into 2 groups. α-Mannosidases in Class 1 are (1,2)-linkage specific enzymes like golgi mannosidase I, whereas α-mannosidases in Class 2, like lysosomal α-mannosidase I, can hydrolyze (1,2), (1,3) and (1,6) linkages (Daniel et al., Glycobiol. 4:551-566 (1994)).

In higher plants, the level of α-mannosidase has been reported to increase during seed germination. The α-mannosidase levels have also been reported to increase during ripening of some fruit and to be involved in the processing of oligosaccharide derivatives that participate in the synthesis of some glycoproteins.

β-mannosidase (EC 2.3.1.25) liberates D-mannose from synthetic substrates and some natural substrates, such as, D-manno-oligosaccharides and D-mannose-containing glycopeptides. β-mannosidase has been reported as an exohydrolase which cleaves β-D-(1,4)-linked mannosyl groups from the non-reducing end of their substrates. β-mannosidase presence has been reported in mammalian systems and higher plants (Percheron, Bull. Acad. Natl. Med. 179:881-892 (1995)).

Xylose reductase catalyzes the conversion of xylose to ethanol. Xylose reductase has been isolated from yeast (Amore et al., Gene 109:89-97 (1991)). Xylose reductase belongs to the aldo-keto reductase superfamily (Bohren et al., J. Biol. Chem. 264:9547-9551 (1989)). The aldo-keto reductases are usually monomeric proteins. Aldo-keto reductases metabolize various substrates ranging from aliphatic and aromatic aldehydes to polycyclic aromatic hydrocarbons. Members of the aldo-keto reductase superfamily have been reported to exhibit amino acid sequence identity. It has been reported that sequence alignment studies have revealed a strict conservation of amino acid sequence at 11 positions in the primary structure of all of the reported proteins. Aldo-keto reductases have been reported to maintain a barrel scaffold structure when modulating substrate specificity through loop modifications. Xylose reductase has been reported from Neurospora crassa (Rawat and Rao, Biochem. Biophys. Acta 1293:222-230 (1996)). A tryptophan residue has also been reported to be involved in the NADPH binding. The presence of a lysine residue, essential for the xylose reductase activity and conformation, has also been reported (Rawat and Rao, Biochim. Biophys. Acta 246:344-349 (1997)). The presence of a cysteine residue that plays a role in the enzyme catalysis has been reported in flouresence studies (Rawat and Rao, Biochim. Biophys. Acta 246:344-349 (1997)).

Glucose dehydrogenase (EC 1.1.1.47) catalyzes an NADP dependent oxidation of glucose to gluconic acid. Glucose dehydrogenase is a quinoprotein as it has pyrrolo-quinoline quinone as its prosthetic group. Aerobic bacteria which do not have a glycolytic pathway or a phosphotransferase system, oxidize glucose by using glucose-dehydrogenase. Glucose-dehydrogenase is a membrane bound enzyme which catalyzes periplasmic oxidation of glucose to gluconic acid (Anthony, Quinoproteins and Energy Transduction, Anthony (ed.), Academic Press, London, pp 293-316, (1988)). Gluconic acid is then further oxidized or released into the growth medium. Some strains of Acinetobacter calcoaceticus are unable to oxidise gluconic acid, although they have an active glucose dehydrogenase. Some enteric bacteria, such as Escherechia coli, are only able to oxidise glucose if provided with pyrrolo-quinoline quinone in the growth medium (Hommes et al., FEMS Microbiol. Lett. 24:329-333 (1984); Neijssel et al., FEMS Microbiol. Lett. 20:35-39 (1983)).

Glucose dehydrogenase has been reported to be reconstituted by the addition of pyrrolo-quinoline quinone to the apo-enzyme (Duine et al., Eur. J. Biochem. 108:187-192 (1980)). In certain bacteria, where glucose dehydrogenase occurs, the products of the catalytic reaction are not further metabolized. Glucose dehydrogenase has been reported to provide energy from glucose (Neijssel et al., In: PQQ and Quinoproteins, Jongejan and Duine (eds.), pp. 57-68 (1989)). Glucose dehydrogenase was first isolated and purified from Acinetobacter calcoaceticus (Hauge, J. Biol. Chem. 239:3630-3639 (1964)).

Ubiquinone has been reported to be an electron acceptor for glucose dehydrogenase in Pseudomonas and Acinetobacter (Matsushita et al., Agric. Biol. Chem. 46: 1007-1011 (1982); Matsushita et al., J. Bact. 169:205-209 (1987); Matsushita et al., J. Biochem. 105:633-637 (1989)). It has been reported that glucose dehydrogenase from Acinetobacter calcoaceticus and E. coli is a monomer of 87 kDa in size (Cleton-Jansen et al., Nucleic Acids Res. 16:6228 (1988); Cleton-Jansen et al., J. Bact. 172:6308-6315 (1990)). The involvement of Mg²⁺ and Ca²⁺ in the binding of pyrrolo-quinoline quinone to the apoenzyme has also been reported (Imanaga, In: PQQ and Quinoproteins, Jongejan et al. (eds.), pp 87-96 (1989)). A periplasmic form and another soluble form of glucose dehydrogenase has also been reported (Cleton-Jansen et al., Mol. Gen. Genet. 217:430-436 (1989); Dokter et al., Biochem. J. 239:163-167 (1986)).

8. Phytic Acid Pathway

myo-Inositol-1-phosphate (mIP6) is synthesized from glucose-6-phosphate with the addition of five phosphates using ATP as a donor yielding mIP6 (Bewley and Black, Seeds: Physiology of Development and Germination, 2^{nd} Ed. Plenum Press, NY (1994); Sasakawa et al., Biochem. Pharmacol. 50:137-146 (1995)). Characterized systems for phytic acid synthesis include the cellular slime mold, Dictyostelium, (Stephens and Irvine, Nature 346:500-583 (1990); Stephens et al., J. Biol. Chem. 268:4009-4015 (1993); van Haastert and van Dijken, FEBS Lett. 410:39-43 (1997)) and the duckweed, Spirodela polyrhiza (Brearley and Hanke, Biochem. J. 314:215-227 (1996)). In both systems, the biosynthetic pathway has been inferred by identification of metabolic intermediates in cell-free extracts. Enzymes reported to be involved in the synthesis of phytic acid are myo-Inositol-1-phosphate synthase (EC 5.5.1.4) and a series of ATP-dependent kinases.

myo-Inositol-1-phosphate synthase catalyzes the conversion of glucose-6-phosphate to 1L-myo-Inositol-1-phosphate in the presence of NAD⁺. myo-Inositol-1-phosphate synthase has been reported from a number of species including Sacharomyces cerevisiae (Dean-Johnson and Henry, J. Biol. Chem. 264:1274-1283 (1989); Katsoulou et al., Yeast 12:789-797 (1996)), Candida albicans (Klig et al., Yeast 10:789-800 (1994)), Arabidopsis thialana (Johnson, Plant Physiol. 105:1023-1024 (1994)), grapefruit (Abu-Abied and Holland, Plant Physiol. 106:1689-1689 (1994)) and Spirodela polyrhiza (Smart and Fleming, Plant J. 4:279-293 (1993)). ATP-dependent kinases have been reported from the identification of metabolic intermediates in Dictostelium and Spirodela polyrhiza.

Phytase (EC 3.1.3.8), also known as myo-inositol hexakisphosphate hydrolase, is found widely in nature and catalyzes the conversion of phytic acid to inositol and orthophosphate, via penta-, quatra-, tri-, di- and monophosphates (Reddy et al., Advances in Food Research 28:1-92 (1982)). Phytases have been isolated and characterized from bacteria, (Patwardhan, Biochem. J. 31:695 (1937)) fungi, (Patwardhan, Biochem. J. 31:695 (1937); Piddington et al., Gene 133:55-62 (1993); van Hartingsveldt et al., Gene 127:97-94 (1993); Ullah, Prep. Biochem. 18:459-471 (1988)), cereals (Singh and Sedeh, Cereal Chem. 56:267 (1979); Nagai and Funahashi, Agric. Biol. Chem. 26:794 (1962); Lim and Tate, Biochim. Biophys. Acta 302:316-315 (1973); Suzuki et al., Bull. Coll. Agric. Tokyo Imp. Univ. 7:495-512 (1907); Yoshida et al., Agric. Biol. Chem. 39:289 (1975)), beans (Lolas and Markakis, J. Food Sci. 42:1094-1098 (1977); Mandal and Biswas, Plant Physiol. 45:4-9 (1970); Mandal et al., Phytochemistry 11:495 (1972); Maiti et al., Phytochem. 13:1047 (1974); Maiti and Biswas, Phytochem. 18:316 (1979); Gibbins and Norris, Biochem. J. 86:67 (1963); Chang, Ph.D. dissertation, Univ. of California, Berkley (1975)); animal (Bitar and Reinhold, Biochim. Biophys. Acta 268:442 (1972)) and human (Bitar, Biochim. Biophys. Acta 268:442-452 (1972)). A plant phytase from corn has also been isolated (Maugenest et al., Biochem. J. 322:511-517 (1997)).

Isolation and expression of fungal phytase, production of enzymes in seeds and expression of fungal phytase in plants have been reported in U.S. Pat. Nos. 5,436,156; 5,543,576, and 5,593,963. Low phytic acid mutants of corn have been reported in U.S. Pat. No. 5,689,054.

C. Amino Acid Pathways

Living organisms differ considerably with respect to their ability to synthesize different amino acids. For example, human being can only synthesize 10 of the 200 amino acids required as building blocks for protein biosynthesis. In contrast, higher plants can make all the amino acids required for protein biosynthesis. Microorganisms differ widely in their capacity to synthesize amino acids.

1. Methoinine Biosynthesis Pathway

The amino acid, L-methionine, is synthesized in higher plants via a pathway that starts with L-aspartate. This pathway has been studied (Azevedo et al., Phytochemistry 46:395-419 (1997)). L-methionine is one of four so-called aspartate-derived amino acids (along with L-lysine, L-threonine, and L-isoleucine) (Miflin et al., IN: Nitrogen Assimilation in Plants, Hewitt et al., (eds.), Academic Press, New York, p. 335 (1997); Bryan, In: The Biochemistry of Plants, Miflin (ed.), Academic Press, New York, p. 403 (1980); Lea et al., In: The Chemistry and Biochemistry of Amino Acids, Barrett et al. (eds.), London, 5:197 (1985); Bryan, In: The Biochemistry of Plants, Miflin et al., (eds.), Academic Press, San Diego, 16:161 (1990)).

In plants, the pathway leading to methionine biosynthesis typically includes the following enzymes: aspartate kinase (EC 2.7.2.4), aspartate-semialdehyde dehydrogenase (EC 1.2.1.11), homoserine dehydrogenase (EC 1.1.1.3), homoserine kinase (EC 2.7.1.39), cystathionine gamma-synthase (EC 4.2.99.9), cystathionine beta-lyase (EC 4.4.1.8), and methionine synthase (EC 2.1.1.14). Some higher plants and microbes utilize alternative enzymatic reactions for one or more steps of the pathway, as noted below.

Aspartate kinase catalyzes the first reaction of the pathway in which aspartate is converted to β-aspartyl phosphate. This enzyme has been isolated and characterized from plant sources including maize, barley, carrot, pea, and soybean. These studies have revealed that there are multiple isoenzymes of aspartate kinase, and the isoenzymes differ with respect to both feedback inhibition sensitivity and expression profile (tissue and developmental stage). Feedback inhibition is mediated by lysine and threonine. Transgenic plants which express an unregulated aspartate kinase have demonstrated increased flux through the aspartate pathway. Pathway regulation is reported to be exerted, at least in part, via control of this enzyme's activity.

Aspartate semialdehyde dehydrogenase catalyses the second pathway reaction and converts β-aspartyl phosphate to aspartate semialdehyde via an NADPH-dependent reaction. Gengenbach et al., Crop Science 18:472-476 (1978) report the isolation of aspartate semialdehyde dehydrogenase from maize suspension culture cells. These suspension cultures did not exhibit feedback inhibition of the enzyme in the presence of aspartate-derived amino acids, with the exception of methionine, for which some feedback sensitivity was observed. Aspartate semialdehyde dehydrogenase enzyme activity has been detected in maize shoot, maize root, and maize kernel (Gengenbach et al., Crop Science 18:472-476 (1978)).

Homoserine dehydrogenase catalyzes the next step of the pathway in which homoserine is generated from aspartate semialdehyde in a reaction requiring NADH or NADPH. Homoserine dehydrogenase enzyme has been studied in higher plants and multiple isoenzyme forms have been reported (Bryan et al., Biochemistry and Biophysics Research Communications 41:1211-1217 (1970); Gengenbach et al., Crop Science 18:472-476 (1978); Dotson et al., Plant Physiology 91:1602-1608 (1989); Dotson et al., Plant Physiology 93:98-104 (1989); Azevedo et al., Phytochemistry 31:3725-3730 (1992); Azevedo et al., Phytochemistry 31:3731-3734 (1992); Brennecke et al., Phytochemistry 41:707 (1996); Aarnes, Plant Science Letters 9:137-145 (1977); Bright et al., Biochemical Genetics 200:229-243 (1982); Aruda et al., Plant Physiology 76:442-446 (1984); Lea et al., In: Barley: Genetics, Molecular Biology and Biotechnology, Shewrey (ed.), CAB International, Oxford, p. 181 (1992); Davies et al., Plant Science Letters 9:323-332 (1977); Davies et al., Plant Physiology 62:536-541 (1978); Matthews et al., Zeitschrift für Naturforschung, Section Bioscience 34:1177-1185 (1979); Relton et al., Biochimica et Biophysica Acta 953:48-60 (1988); Aarnes et al., Phytochemistry 13:2717-2724 (1974); Lea et al., FEBS Letters 98:165-168 (1979); Matthews et al., Canadian Journal of Botany 57:299-304 (1979)). The isoenzymes have been found to differ with respect to sensitivity to threonine-mediated feedback inhibition, with both sensitive and insensitive forms being isolated from maize suspension cultures and seedlings (Miflin et al., In: Nitrogen Assimilation of Plants 335 (1979); Bryan, In: The Biochemistry of Plants, Miflin (ed.), Academic Press, New York, 5:403 (1980)).

There is evidence that plants also possess a bifunctional enzyme with both aspartate kinase and homoserine dehydrogenase activities (Lea et al., The Chemistry and Biochemistry of Amino Acids 197 (1985); Bryan, In: The Biochemistry of Plants, Miflin (ed.), Academic Press, New York, 5:161 (1990)). Clones of these bifunctional enzymes have been isolated from Arabidopsis thaliana (Giovanelli et al., In: The Biochemistry of Plants, Miflin (ed.), Academic Press, New York, p. 453 (1990)) carrot (Giovanelli et al., Plant Physiology 90:1584-1599 (1989)), maize (Singh et al., Amino Acids 7:165-168 (1994)) and soybean (Matthews et al., In: Biosynthesis and Molecular Regulation of Amino Acids in Plants, Singh et al. (eds.), American Society of Plant Physiologists, Rockville, Md., p 294 (1992)).

The next enzymatic step leading to methionine biosynthesis in higher plants is the final common reaction shared by other amino acid end products (threonine and isoleucine). The reaction is catalyzed by homoserine kinase resulting in the generation of O-phosphohomoserine from homoserine, with ATP serving as the phosphate donor. Homoserine kinase has been purified to varying degrees from multiple higher plant sources (Galili, The Plant Cell 7:899-906 (1995); Rees et al., Biochemical Journal 309:999-1107 (1995); Bryan et al., Biochemistry and Biophysics Research Communications 41:1211-1217 (1970); Gengenbach et al., Crop Science 18:472-476 (1978); Dotson et al., Plant Physiology 91:1602-1608 (1989); Dotson et al., Plant Physiology 93:98-104 (1989)). Homoserine kinase isolated from barley and wheat did not exhibit feedback inhibition by aspartate-derived amino acids (Gengenbach et al., Crop Science 18:472-476 (1978); Dotson et al., Plant Physiology 93:98-104 (1989)). There is some evidence for feedback regulation of this enzyme in the dicots, pea (Rees et al., Biochemical Journal 309:999-1007 (1995)) and radish (Bryan et al., Biochemistry and Biophysics Research Communications 41:1211-1217 (1970)). Bacterial and yeast homologues have been reported (Azevedo et al., Phytochemistry 31:3725-3730 (1992); Azevedo et al., Phytochemistry 31:3731-3734 (1992); Brennecke et al., Phytochemistry 41:707 (1996); Aarnes, Plant Science Letters 9:137-145 (1977)).

O-acetylhomoserine and O-oxalylhomoserine are generated as alternatives to O-phosphohomoserine in Pisum sativum and Lathyrus sitivus, respectively (Thomas and Surdin-Kerjan, Microbiol. Mol. Biol. Rev. 61:503-532 (1997)). Enteric bacteria use O-succinylhomoserine instead of O-phosphohomoserine, while several gram-positive bacteria, yeasts and fungi use O-acetylhomoserine (formed using homoserine O-acetyltransferase, EC 2.3.1.31, (Thomas and Surdin-Kerjan, Microbiol. Mol. Biol. Rev. 61:503-532 (1997)).

In yeast, sulfur is incorporated into carbon chains by the O-acetylhomoserine sulfhydrylase (EC 4.2.99.10) reaction, which generates homocysteine from O-acetylhomoserine. O-acetylhomoserine sulfhydrylase has been purified to homogeneity and shown to be a homotetramer with a molecular weight of 200,000 and to bind four molecules of pyridoxal phosphate (Thomas and Surdin-Kerjan, Microbiol. Mol. Biol. Rev. 61:503-532 (1997)).

In yeast, cysteine is synthesized from homocysteine via two successive enzymatic steps, beta addition and gamma elimination. Cystathionine beta-synthase (EC 4.2.1.22) catalyzes the first reaction in which cystathionine is generated from homocysteine and serine. In S. cervisiae, cystathionine beta-synthase is encoded by the STR4 gene. (Thomas and Surdin-Kerjan, Microbiol. Mol. Biol. Rev. 61:503-532(1997)). Cystathionine gamma-lyase (EC 4.4.1.1) catalyzes the gamma cleavage of cystationine, which is the second reaction leading to cysteine biosynthesis from homocysteine. This enzyme, encoded by STR1, has been purified to homogeneity and has a molecular weight of about 194,000 (Thomas and Surdin-Kerjan, Microbiol. Mol. Biol. Rev. 61:503-532 (1997)).

Cystathionine γ-synthase catalyzes the first reaction which is unique to methionine biosynthesis, thereby committing aspartate pathway flux toward this amino acid. In this reaction, O-phosphohomoserine and cysteine serve as common substrates in higher plants for the production of cystathionine. No isoenzymes of cystathionine γ-synthase from plants sources have been characterized. No feedback inhibition of this enzyme by aspartate-derived amino acids has been reported (Bright et al., Biochemical Genetics 20:229-243 (1982); Arruda et al., Plant Physiology 76:442-446 (1984)). The enzyme has been reported to be sensitive to product inhibition by orthophosphate (Lea et al., Barley: Genetics, Molecular Biology and Biotechnology, Shewrey (ed.), CAB International, Oxford, 181 (1992); Davies et al., Plant Science Letters 9:323-332 (1977)). The gene for cystathionine γ-synthase has been cloned from Arabidopsis thaliana (Davies et al., Plant Physiology 62:536-541 (1978). There is evidence suggesting that flux to methionine is modulated via regulation of cystathionine-synthase (Matthews et al., Zeitschrift für Naturforschung, Section Bioscience 34:1177-1185 (1979-2724 (1974); Lea et al., FEBS Letters 98:165 (1979)).

Cystathionine beta-lyase catalyzes the next reaction in the biosynthesis of methionine. This reaction generates homocysteine, pyruvate, and ammonia from the enzymatic decomposition of cystathionine. Evidence for isoenzymes which differ with respect to cellular localization have been reported for barley (Matthews et al., Canadian Journal of Botany 57:299-304 (1979)), and spinach (Rognes et al., Nature 287:357-359 (1980)).

Methionine synthase generates methionine from homocysteine by a methylation reaction and thus represents the final step of the methionine biosynthetic pathway. Methionine synthase is also sometimes referred to as 5-methyltetrahydropteroyltriglutamate-homocysteine-S-methyltransferase. N-methyltetrahydrofolate serves as the methyl donor in this reaction, which occurs in the absence of cobalamin (Giovanelli et al., Plant Physiology 90:1577-1583 (1989); Green et al., Crop Science 14:827-830 (1974)).

2. Methionine Degradation Pathway

Plants contain a pathway for the degradation of L-methionine. This degradation pathway includes the following enzymes: methionine adenosyltransferase (EC 2.5.1.6), methionine S-methyltransferase (EC 2.1.1.12), adenosylmethionine hydrolase (EC 3.3.1.2), homocysteine S-methyltransferase (EC 2.1.1.10) and S-adenosyl-methionine decarboxylase (EC 4.1.1.50).

The reported first step in the catabolism of methionine is the ATP-dependent conversion to S-adenosylmethionine (AdoMet), which is catalyzed by the enzyme methionine adenosyltransferase, also known as S-adenosylmethionine synthetase. Methionine adenosyltransferase enzyme has been characterized from several plant sources (Aarnes, Plant Science Letters 10:381 (1977); Mathur et al., Biochimia and Biophysica Acta 1078:161-170 (1991); Kim et al., Journal of Biochemical and Molecular Biology 28:100 (1995)) and nucleic acid molecules (genomic and cDNA) have also been obtained from a variety of sources (Izhaki et al., Plant Physiology 108:841-842 (1995); Espartero et al., Molecular Biology Plant 25:217-237 (1994)). Regulation of methionine adenosyltransferase activity has been observed for the enzyme from Glycine max (soybean). In Glycine max, methionine adenosyltransferase was reportedly inhibited by S-adenosylmethionine (Kim et al., Journal of Biochemical and Molecular Biology 28:100 (1995). Studies have also reported that the levels of methionine adenosyltransferase appear to fluctuate in response to hormonal or environmental conditions such as gibberellic acid (Mathur et al., Biochimica and Biophysica Acta 1162:289-290 (1993); Mathur et al., Biochimica and Biophysica Acta 1137:338-348 (1992)), salt stress (Espartero et al., Molecular Biology Plant 25:217-227 (1994)), and wounding (Kim et al., Plant Cell Reports 13:340 (1994)). It has also been reported that methionine adenosyltransferase may play a role in the lignification process (Peleman et al., Plant Cell 1:81 (1989)).

AdoMet is further catabolized by several enzymes and has been reported to serve a variety of metabolic functions including that of a methyl donor (Cossins, In: The Biochemistry of Plants 11:317, Devis (ed.), Academic Press, San Diego (1987)) that of a precursor for polyamine biosynthesis (Tiburico et al., The Biochemistry of Plants 16:283 (1990)) and that of a precursor for ethylene biosynthesis (Kende, Plant Physiology 91:1-4 (1989); Flurh et al., Critical Review of Plant Science 15:479 (1996)). In each case, enzymes are present to regenerate methionine from the sulfur-containing backbone resulting in no net loss of methionine.

An enzyme involved in AdoMet catabolism is adenosylmethionine hydrolase which converts AdoMet to methylthioadenosine and L-homoserine. L-homoserine is further metabolized during the biosynthesis of polyamines and ethylene and methylthioadenosine is recycled to methionine.

Another enzyme for which AdoMet is a substrate for is homocysteine S-methyltransferase. Homocysteine S-methyltransferase catalyzes the combination of AdoMet, with L-homocysteine to produce both S-adenosyl-L-homocysteine and L-methionine. Another enzyme has been described which generates S-adenosyl-L-homocysteine from AdoMet. This enzyme is called methionine S-methyltransferase, and it catalyzes the reaction in which S-adenosyl-L-homocysteine reacts with L-methionine to generate S-adenosyl-L-homocysteine and S-methyl-L-methionine. AdoMet can also be decarboxylated by adenosyl methionine decarboxylase, which generates (5-deoxy-5-adenosyl)(3-aminopropyl) methylsulfonium salt.

S-adenosyl-L-homocysteine is removed by S-adenosyl-L-homocysteine hydrolase (EC 3.3.1.1) to yield homocysteine and adenosine. This enzyme has been characterized in tobacco and parsley and its gene is cloned. The predicted amino acid sequence is highly homologous to that of S-adenosyl-L-homocysteine from various organisms (Ravanel et al., Proc. Natl. Acad. Sci. (U.S.A.) 93:7805-7812 (1998)).

Another enzyme has been described which generates S-adenosyl-L-homocysteine from AdoMet. This enzyme is called methionine S-methyltransferase, and it catalyzes the reaction in which S-adenosyl-L-homocysteine reacts with L-methionine to generate S-adenosyl-L-homocysteine and S-methyl-L-methionine. AdoMet can also be decarboxylated by adenosyl methionine decarboxylase, which generates (5-deoxy-5-adenosyl) (3-aminopropyl) methylsulfonium salt.

3. Lysine Pathway

L-lysine is synthesized in higher plants via a pathway that starts with L-aspartate. (Azevedo et al., Phytochemistry 46:395-419 (1997)). It is one of the four so-called aspartate-derived amino acids (along with L-methionine, L-threonine, and L-isoleucine) (Miflin et al., Nitrogen Assimilation in Plants, Hewitt et al. (eds.), Academic Press, New York, p. 335 (1997); Bryan, The Biochemistry of Plants, Miflin (ed.), Academic Press, New York, 403 (1980); Lea et al., The Chemistry and Biochemistry of Amino Acids, Barrett et al. (eds.), London, 5:197 (1985); Bryan, The Biochemistry of Plants, Miflin et al. (eds.), Academic Press, San Diego, 16:161 (1990)).

Aspartate kinase (EC 2.7.2.4) and aspartate-semialdehyde dehydrogenase (EC 1.2.1.11) have been reported to catalyze the formation of aspartate semialdehyde. Aspartate semialdehyde has been reported to be the common precursor for the synthesis of L-lysine, L-methionine, L-threonine, and L-isoleucine. Enzymes that are reported to be specific for the synthesis of lysine include dihydrodipicolinate synthase (EC 4.2.1.52), dihydrodipicolinate reductase (EC 1.3.1.26), piperidine dicarboxylate acylase (EC 2.3.1.117) acyldiaminopimelate aminotransferase (EC 2.6.1.17), acyldiaminopimelate deacylase (EC 3.5.1.18), diaminopimelate epimerase (EC 5.1.1.7), and diaminopimelate decarboxylase (EC 4.1.1.20).

Aspartate kinase catalyzes the first reported reaction of the pathway in which aspartate is converted to β-aspartyl phosphate. Aspartate kinase has been reported from several plant sources including maize, barley, carrot, pea, and soybean (Azevedo et al., Phytochemistry 46:395-419 (1997)). It has been reported that there are multiple isoenzymes of aspartate kinase, and that the isoenzymes differ with respect to both feedback inhibition sensitivity and expression profile (tissue and developmental stage). Feedback inhibition has been reported to be mediated by lysine and threonine. Transgenic plants which express an unregulated aspartate kinase enzyme have been reported to have increased flux through the aspartate pathway. Pathway regulation has been reported to be exerted, at least in part, via control of this enzyme's activity.

Aspartate-semialdehyde dehydrogenase catalyses the second reported reaction of the lysine biosynthesis pathway and converts β-aspartyl phosphate to aspartate semialdehyde via an NADPH-dependent reaction. Gengenbach et al. (Crop Science 18:472-476 (1978)), reported the isolation of aspartate-semialdehyde dehydrogenase from maize suspension culture cells. These suspension cultures have reported to not exhibit feedback inhibition of the enzyme in the presence of aspartate-derived amino acids, with the exception of methionine, for which some feedback sensitivity is observed. Aspartate-semialdehyde dehydrogenase enzyme activity has been reported in maize shoot, root, and kernel tissues (Gengenbach et al., Crop Science 18:472-476 (1978)).

Homoserine dehydrogenase (EC 1.1.1.3) catalyzes the conversion of aspartate semialdehyde into homoserine in a reaction requiring NADH or NADPH. Multiple isozyme forms of homoserine dehydrogenase enzyme have been reported (Bryan et al., Biochemistry and Biophysics Research Communications 41:1211-1217 (1970); Gengenbach et al., Crop Science 18:472-476 (1978); Dotson et al., Plant Physiology 91:1602-1608 (1989); Dotson et al., Plant Physiology 93:98-104 (1989); Azevedo et al., Phytochemistry 31:3725-3730 (1992); Azevedo et al., Phytochemistry 31:3731-3734 (1992); Brennecke et al., Phytochemistry 41:707 (1996); Aarnes, Plant Science Letters, 9:137-145 (1977); Bright et al., Biochemical Genetics 20:229-243 (1982); Arruda et al., Plant Physiology 76:442-446 (1984); Lea et al., Barley: Genetics, Molecular Biology and Biotechnology, Shewrey (ed.), CAB International, Oxford, p. 181 (1992); Davies et al., Plant Science Letters 9:323-332 (1977); Davies et al., Plant Physiology 62:536-541 (1978); Matthews et al., Zeitschrift für Naturforschung, Section Bioscience 34:1177-1185 (1979); Relton et al., Biochimica et Biophysica Acta 953:48-60 (1988); Aarnes et al., Phytochemistry 13:2717-2724 (1974); Lea et al., FEBS Letters 98:165-168 (1979); Matthews et al., Canadian Journal of Botany 57:299-304 (1979)). Homoserine dehydrogenase isoenzymes have been reported to differ with respect to sensitivity to threonine-mediated feedback inhibition. Both sensitive and insensitive forms have been reported from maize suspension cultures and seedlings (Miflin et al., Nitrogen Assimilation of Plants 335 (1979); Bryan, The Biochemistry of Plants, Miflin (ed.), Academic Press, New York, 5:403 (1980)).

Dihydrodipicolinate synthase catalyses the first reported reaction committed to lysine biosynthesis. Dihydrodipicolinate synthase has been reported to be associated with the regulation of lysine biosynthesis. Dihydrodipicolinate synthase has also been reported to catalyze the condensation of pyruvate and aspartate semialdehyde into dihydrodipicolinate (Frish et al., Molecular and General Genetics 288:287-291 (1991); Wallsgrove et al., Phytochemistry 20:2651-2655 (1981); Dereppe et al., Plant Physiology 98:813-821 (1992); Ghislain et al., Planta 180:480-486 (1990)). Dihydrodipicolinate synthase clones have been reported from poplar, wheat, Arabidopsis and soybean. Clones from maize and wheat have been reported to exhibit homology (Azevedo et al., Phytochemistry 46:395-419 (1997)).

Lysine is reported to be a competitive inhibitor with respect to aspartate semialdehyde but not with respect to pyruvate (Kumpaisal et al., Plant Physiology 85:145-151 (1987)). Three amino acid residues within one region of dihydrodipicolinate synthase have been reported to be involved in the regulation of the feedback inhibition property of the maize dihydrodipicolinate synthase enzyme (Shaver et al., Proc. Natl. Acad. Sci. (U.S.A.) 93:1962-1966 (1996)).

Dihydrodipicolinate reductase and diaminopimelate decarboxylase, have also been reported to be involved in lysine biosynthesis.

Dihydrodipicolinate reductase catalyses the reduction of dihydrodipicolinic acid to tetrahydrodipicolinic acid. A partially purified dihydrodipicolinate reductase from maize kernel has been reported to be inhibited by compounds similar to dihydrodipicolinic acid (Tyagi et al., Plant Physiol. 73:687-691 (1983)).

Acyldiaminopimelate deacylase (also known as succinyl-diaminopimelate desuccinylase) converts tetrahydrodipicolinic acid to diaminopimelic acid (Lalonde, Mol. Microbiol. 11:273-280 (1994); Edwards, Mol. Gen. Genet. 247:189-198 (1995); Berges et al., J. Med. Chem. 29:89-95 (1986)).

An E. coli diaminopimelate epimerase has a reported molecular weight of 34 kD (Wiseman and Nichols, J. Biol. Chem. 259:8907-8914 (1984); Higgins et al., Eur. J. Biochem. 186:137-143 (1989); Richaud et al., J. Bacteriol. 169:1454-1459 (1987)). Diaminopimelate epimerase has been reported to exchange the alpha protons of the substrates DL- and LL-diaminopimelic acid in a two base reaction (Wiseman and Nichols, J. Biol. Chem. 259:8907-8914 (1984)).

Diaminopimelate decarboxylase (EC 4.1.1.20) catalyzes the last reported step in the lysine biosynthesis pathway and converts meso-diaminopimelic acid to lysine by a decarboxylation reaction. Diaminopimelate decarboxylase has been reported to be a chloroplast-localized enzyme (Mazelis et al., FEBS Letters 84:236-240 (1977)). Diaminopimelate decarboxylase has been reported in Lemna perpusilla, Vicia faba, and maize (Shimura et al., Biochem. Biophys. Acta 118:396-401 (1996); Mazelis et al., FEBS Letters 84:236-240 (1976); White et al., Biochem. J. 96:75-80 (1965)).

It has been reported, that in radiolabelled lysine fed plants, lysine is catabolized via saccharopine (Brandt, FEBS Letters 52:288-291 (1975); Arruda et al., Plant Physiol. 69:988-999 (1982)). The first two reported steps in lysine catabolism are catalyzed by the bifunctional enzyme, lysine-ketoglutarate reductase/saccharopine dehydrogenase (EC 1.5.1.8). Lysine-ketoglutarate reductase/saccharopine dehydrogenase has been reported to be a homodimer of 260 kD (Goncalves-Butruille et al., Plant Physiol. 110:765-771 (1996)). Lysine-ketoglutarate reductase and saccharopine dehydrogenase activities are reported to reside in adjacent domains and catalyze the sequential steps of the enzyme reaction. It has been reported that lysine-ketoglutarate reductase activity, in developing tobacco seeds, can be induced by treatment with exogenous lysine (Karchi et al., Proc. Natl. Acad. Sci. (U.S.A.) 91:2577-2581 (1994)).

4. Arginine Pathway

Arginine serves multiple physiological functions during the growth and development of higher plants. One of the physiological functions of arginine is the maintenance of nitrogen metabolism. In addition to its role as an amino acid constituent of proteins, arginine also appears to play a role as a plant storage molecule for nitrogen (Thompson, The Biochem. of Plants, Vol. 5 Miflin (ed.), Academic Press, New York. (1980)). For example, in the protein-rich soybean cotyledon, arginine was found to contribute 18% of total protein nitrogen throughout development (Micallef et al., Plant Physiol. 90:624-630 (1989)), and levels as high as 40% of seed protein nitrogen have been reported (Vaneteen et al., Agric. Food Chem. 5:399-410 (1963)). Moreover, arginine comprises from 50% to 90% of the free amino acid pool found in developing soybean cotyledons (Micallef, et al., Plant Physiol. 90:624-630 (1989)), developing pea cotyledons (Deruiter et al., Plant Physiol. 73:525-528 (1983)), cotton seeds (Capdevila et al., Plant Physiol. 59:268-273 (1977)), fruit trees (Oland, Plant Physiol. 12:594-646 (1959)), grape vines (Kliewer et al., Am. J. Enol. Vitic. 25:111-118 (1974)), and flower bulbs (Boutin, Eur. J. Biochem. 127:237-243 (1982)). The role of arginine as a nitrogen transport molecule in plants has also been reported (Oland, Physiol. Plant 12:594-646 (1959).

In addition to maintaining nitrogen levels, arginine metabolism is also associated with the synthesis of secondary metabolites in plants (Beevers, Nitrogen Metabol. in Plants, Edward Arnold, London. p. 62 (1976)). For example, the diamine, putrescine, and the polyamines, spermidine and spermine, are generated from the catabolism of arginine or ornithine, which itself is an intermediate of arginine biosynthesis (Goodwin et al., Intro. to Plant Biochem. 2^{nd} edition. Pergamon Press, Oxford (1983)). A role for polyamines in such processes as stress tolerance, cell division, and organogenesis has been reported based on correlations between polyamine levels and the plant's physiological state (Walden et al., Plant Physiol. 113:1009-1013 (1997)). Ornithine is also involved in the synthesis of alkaloids (Goodwin et al., Introduction to Plant Biochem. 2^{nd} ed., Pergamon Press, Oxford (1983). In addition, another intermediate, carbamoyl phosphate, contributes to the de novo synthesis of pyrimidine nucleotides, which are required for DNA synthesis (Jones, Annu. Rev. Biochem. 49:253-279 (1980); Ross, The Biochem. of Plants, Vol. 6, Protein and Nucleic Acids, Marcus (ed.), Academic Press, New York, p. 169-205. (1981)).

Arginine is derived from glutamate via a series of reactions that are common to the non-enteric bacteria, fungi, yeast, green algae, and higher plants reported to date (Thompson, The Biochem. of Plants, Vol. 5, Miflin (ed.) Academic Press, New York, p. 375 (1980); McKay et al., Plant Sci. Letters 9:189-193 (1977)). In plants, ¹⁴C-labeling studies have provided evidence for a role for glutamate as the precursor for arginine biosynthesis (McConnell, Can. J. Biochem. Physiol. 37:933-936 (1959)); Morris et al., Plant Physiol. 59:684-687 (1977)). The arginine biosynthetic pathway can be divided into two main sets of enzymatic reactions: (1) those reactions which convert glutamate to ornithine and (2) those reactions which convert ornithine to arginine.

Glutamate is converted to ornithine via a series of reactions involving acetylated intermediates. The sequence of intermediates is as follows (in order): glutamate, N-acetylglutamate, N-acetylglutamate 5-phosphate, N-acetylglutamate 5-semialdehyde, N-acetylornithine, and ornithine (Bryan, The Biochem. of Plants, Vol. 16, Miflin and Lea (eds.) Academic Press, New York. p. 186-187 (1990)). Enzymes which catalyze these conversions are as follows (respectively): acetyl-CoA:glutamate N-acetyltransferase (EC 2.3.1.1), N-acetylglutamate kinase (EC 2.7.2.8), N-acetylglutamate semialdehyde oxidoreductase (EC 1.2.1.38), N-acetylornithine aminotransferase (EC 2.6.1.11), and acetylornithine deacetylase (EC 3.5.1.16) (Gamble et al., J. Biol. Chem. 248:610-618 (1973); Weiss et al., J. Biol. Chem. 248:5403-5408 (1973); O'Neal et al., Biochem. Biophys. Res. Commun. 31:322-327 (1968); Gamborg et al., Exp. Cell Res. 50:151-158 (1968); Gamborg., Plant Physiol. 45:72-375 (1970); Lowry et al., J. Biol. Chem. 193:265-275 (1951)). In addition, the enzyme acetylornithine: glutamate N-acetyltransferase (EC 2.3.1.35) is also involved in the generation of both N-acetylglutamate and ornithine via transfer of the acetyl group from N-acetylornithine to glutamate (Bryan, The Biochem. of Plants, Vol. 16, Miflin and Lea (ed.) Academic Press, New York, p. 186-187 (1990)).

Ornithine is converted to arginine via three enzymatic reactions (Bryan, The Biochem. of Plants, Vol. 16, Miflin and Lea (ed.) Academic Press, New York, p. 186-187 (1990)). The first reported reaction is catalyzed by ornithine carbamoyltransferase (EC 2.1.3.3), and it generates citrulline from the substrates, ornithine and carbamoyl phosphate. Carbamoyl phosphate for this reaction can be generated by the enzyme, carbamoyl phosphate synthetase (EC 6.3.5.5), which uses glutamine, carbon dioxide, and ATP as a substrate (Kolloffel et al., Plant Physiol. 69:143-145 (1982)).

The second reported reaction generates argininosuccinate from citrulline and aspartate (with ATP as an energy donor) and is catalyzed by argininosuccinate synthetase (EC 6.3.4.5). Argininosuccinate is then catabolized to arginine and fumarate by the enzyme, argininosuccinate lyase (EC 4.3.2.1) (Davis, Advances in Enzymol., Vol. 16, Meister (ed.), John Wiley & Sons, New York, p. 247-312; Micallef et al., Plant Physiol. 90:631-634 (1989); Thompson, The Biochem. of Plants, Vol. 5 Miflin (ed) Academic Press, London, p. 375-402 (1980)).

Catabolic enzymes catalyze reactions leading to the recycling of the nitrogen bound by arginine. It has been reported that the levels of arginine catabolic enzymes are significantly elevated during seed germination, and that arginine serves as an important nitrogen source for this process (DeRuiter, Ph.D. Thesis. University of Utrecht (1984); DeRuiter et al., Plant Physiol. 70:313 (1982); DeRuiter et al., Plant Physiol. 73:523 (1983).

Enzymes which catalyze the degradation of arginine in plants include arginase (EC 3.5.3.1), arginine decarboxylase (EC 4.1.1.19), and arginine deiminase (EC 3.5.3.6). Arginase converts arginine to ornithine and urea. Ornithine and urea catabolites can be further metabolized to yield polyamines, alkaloids and ammonia (Kang et al., Plant Physiol. 93:1230-1234 (1990)). Arginine decarboxylase, converts arginine to agmatine, which can then be further processed to form putrescine and polyamines (Borrell et al., Plant Physiol. 109:771-776 (1995); Tabor et al., Microbiol. Rev. 49:81-99 (1985); Pegg, Biochem. J. 234:249-262 (1986)).

Arginine deiminase converts arginine to citrulline and generates ammonia. It has been reported that this activity enables plant cells to utilize arginine as an endogenous nitrogen source (Ludwig, Plant Physiol. 101:429-434 (1993)).

In plants, arginine synthesis is primarily localized to the plastids and the cytosol. Arginine catabolism is primarily located in the mitochondria (Shargool et al., Phytochem. 27:1571-1574 (1988)). It has been reported that such compartmentalization of enzymes enables a cell to sustain ornithine levels destined for arginine biosynthesis physically separated from the catabolically derived ornithine which is earmarked for glutamate and arginine generation (Thompson, The Biochem. of Plants, Vol. 5, Miflin (ed.), Academic Press, London, p. 375-402 (1980). An enzyme that further metabolizes ornithine into glutamate, ornithine aminotransferase, is a mitochondrial enzyme whose levels increase during seed germination (Taylor et al., Biochem. Biophys. Res. Commun. 101:1281-1289 (1981); McKay, Ph.D. Thesis, University of Saskatchewan (1980)).

Localization studies have been reported for arginine biosynthetic pathway enzymes using both soybean cells (protoplasts) and pea leaf tissue cells (Jain et al., Plant Sci. 51:17-20 (1987); Taylor et al., Biochem. Biophys. Res. Commun. 101:1281 (1981); Shargool et al., Can. J. Biochem. 56:273 (1978)). Based on these studies, the following enzymes were localized in the plastids: acetylornithine:glutamate N-acetyltransferase, N-acetylornithine aminotransferase, ornithine carbamoyltransferase, and carbamoyl phosphate synthetase. Cytosolic locations have been reported for acetyl-CoA:glutamate N-acetyltransferase, N-acetylglutamate kinase, argininosuccinate synthetase, and argininosuccinate lyase (Jain et al., Plant Sci. 51:17-20 (1987); Taylor et al., Biochem. Biophys. Res. Commun. 101:1281 (1981); Shargool et al., Can. J. Biochem. 56:273 (1978); DeRuiter, Ph.D. Thesis, University of Utrecht (1984)). Arginase has been reported to exist in the mitochondria of several plants, including broad bean, pea, jackbean, and soybean (Kolloffel et al., Plant Physiol. 55:507 (1975); Taylor et al., Biochem. Biophys. Res. Commun. 101:1281 (1981); Downum et al., Plant Physiol. 73:963 (1983)). Studies using oat leaves have reported that arginine decarboxylase is localized in the plastids (chloroplasts) (Borrell et al., Plant Physiol. 109:771-776 (1995)). It has also been reported that arginine catabolism via arginine iminohydrolase occurs in the chloroplasts of Arabidopsis leaves (Ludwig, Plant Physiol. 101:429-434 (1993)).

The first reported step in the synthesis of arginine is an acetylation of glutamate. This reaction can be catalyzed by either of two enzymes, acetyl-CoA:glutamate N-acetyltransferase (EC 2.3.1.1) or acetylornithine:glutamate N-acetyltransferase (EC 2.3.1.35) (McKay et al., Plant Sci. Letters 9:189-193 (1977); Morris et al., Plant Physiol. 55:960-967 (1975); Morris et al., Plant Physiol. 59:684-687 (1977)). In soybean cell cultures acetylornithine:glutamate N-acetyltransferase is a reported rate-limiting enzyme of ornithine biosynthesis and simultaneously generates ornithine and acetylglutamate from acetylornithine and glutamate. Acetylornithine:glutamate N-acetyltransferase has been reported to be associated with the recycling of acetyl groups in the arginine pathway (Shargool et al., Plant Physiol. 78:796-798 (1985); Shargool et al., Plant Physiol. 78:796-798 (1985); Shargool et al., Phytochem. 27:1571-1574 (1988)). Acetylornithine:glutamate N-acetyltransferase activity has been reported to be common to organisms, with the exception of the Enterobacteriaceae and the archeon Sulfolobus solfataricus, which utilize acetyl ornithase (EC 3.5.1.16) (Shargool et al., Phytochem. 27:1571-1574 (1988); Cunin et al., Microbiol. Rev. 50:314-352 (1986); Van de Casteele et al., J. General Microbiol. 136:1177-1183 (1990)). Acetylglutamate synthesis acetyl-CoA:glutamate N-acetyltransferase has been reported to serve an anapleurotic function in plants by supplementing the activity of the acetylornithine-utilizing acetyltransferase.

Biochemical studies of acetyl-CoA:glutamate N-acetyltransferase (EC 2.3.1.1) and acetylornithine:glutamate N-acetyltransferase (2.3.1.35) from extracts of plant sources and the green alga, Chlorella vulgaris, have reported that the anapleurotic enzyme is inhibited by arginine and that acetylornithine:glutamate N-acetyltransferase is insensitive to the pathway end-product (McKay, Ph.D. Thesis, University of Saskatchewan (1980); Clayton et al., Plant Physiol. 59:684-687 (1977); Clayton et al., Plant Physiol. 55:960-967 (1975)). Kinetic studies of acetyl-CoA:glutamate N-acetyltransferase and acetylornithine:glutamate N-acetyltransferase activities in extracts of sugar beet leaves reported Kₘ values of 2.5 and 0.025 mM for acetyl-CoA and acetylornithine, respectively (Clayton et al., Plant Physiol. 59:684-687 (1977)). Corresponding Kₘ values from Chlorella extracts were found to be 3.2 and 0.2 mM, respectively (Clayton et al., Plant Physiol. 55:960-967 (1975)). In sugar beet extracts, the pH optimum for the acetyl-CoA:glutamate N-acetyltransferase activity was reported to be pH 7.2, while that of the acetylornithine-dependent acetyltransferase was reported to be pH 8.3 (Clayton et al., Plant Physiol. 59:684-687 (1977)). The nucleotide sequences of several microbial genes for these two acetyltransferases have been reported (GenBank database, File Release No. 104, National Center for Biotechnology Information, National Library of Medicine, 38A, 8N805, 8600 Rockville Pike, Bethesda, Md.).

The next reported step of the arginine biosynthetic pathway is the phosphorylation of N-acetylglutamate to form N-acetylglutamate 5-phosphate. ATP serves as the phosphoyl donor in this reaction, which is catalyzed by N-acetylglutamate kinase (EC 2.7.2.8). N-Acetylglutamate kinase has been purified to homogeneity from pea cotyledon (McKay et al., Biochem. J. 195:71-81 (1981)). N-Acetylglutamate kinase has been reported to be oligomeric in nature and composed of two different subunits, with molecular weights of 43 and 53 kDa. N-Acetylglutamate kinase can exist as a dimer or a tetramer, each made up of equal numbers of the two subunits. N-Acetylglutamate kinase exhibits a reported negative cooperativity with respect to acetylornithine and two Kₘ values of 1.9 and 6.2 mM. The reported Kₘ value for ATP was 1.7 mM. It was also reported that this enzyme is inhibited by arginine. In addition, it was reported that this inhibition is relieved by N-acetylglutamate, which can function as a activator (McKay et al., Biochem. J. 195:71-81 (1981); McKay, Ph.D. Thesis, University of Saskatchewan (1980)). Inhibition of N-acetylglutamate kinase activity by arginine was also reported from studies of extracts of the green alga, Chlorella vulgaris (Clayton et al., Plant Physiol. 55:960-967 (1975)). It has also been reported that the N-acetylglutamate kinase reaction represents a key regulatory point in the pathway of arginine biosynthesis in plants (McKay et al., Biochem. J. 195:71-81 (1981); Shargool et al., Phytochem. 27:1571-1574 (1988)). The nucleotide sequences for microbial N-acetylglutamate kinase genes have been reported (GenBank database, File Release No. 104, National Center for Biotechnology Information, National Library of Medicine, 38A, 8N805, 8600 Rockville Pike, Bethesda, Md.).

In the next reported step of arginine biosynthesis, N-acetylglutamate 5-phosphate is converted to N-acetylglutamate 5-semialdehyde via a reaction catalyzed by N-acetylglutamate 5-semialdehyde oxidoreductase (EC 1.2.1.38). NADPH serves as the reducing agent for this reaction. The semialdehyde product of this reaction is then converted to N-acetylornithine by the action of N-acetylornithine aminotransferase (EC 2.6.1.11). In this reaction, glutamate serves as the amino donor and is converted to α-ketoglutarate. A 341-bp sequence representing the 5′ partial sequence of a putative cDNA clone (EST) of the N-acetylornithine aminotransferase gene in Arabidopsis thaliana has been reported (GenBank Accession No. Z97344, Desprez et al.).

The next reported intermediate generated in the arginine biosynthetic pathway is ornithine, and it can be generated via two different reactions. One reaction involves acetyltransferase (EC 2.3.1.35). In the acetyltransferase reaction, an acetyl group of N-acetylornithine is transferred to glutamate, resulting in ornithine. The second reaction which generates ornithine is catalyzed by acetylornithine deacetylase (EC 3.5.1.16). In addition to generating ornithine, this reaction also releases acetate. Acetylornithine deacetylase is reported to be required as a means to complement the initial anapleurotic reaction catalyzed by acetyl-CoA:glutamate acetyltransferase (i.e., when acetyl groups are not recycled in the pathway). A subset of nucleotide sequence from genomic DNA of Arabidopsis thaliana has been reported as the gene for acetylornithine deacetylase (GenBank Accession No. Z34670, Bevan et al.).

Ornithine can be converted to other metabolites besides arginine (e.g., putrescine). A conversion of ornithine to the next pathway intermediate, citrulline, represents the first reported commitment of flux to the arginine end-product. This reaction is catalyzed by ornithine carbamoyltransferase (EC 2.1.3.3) and, in addition to ornithine, involves a second substrate, carbamoyl phosphate. Ornithine carbamoyltransferase has been isolated from plant sources, including maize, wheat, barley, wild oat, oat, sugar beet, soybean, and sicklepod (Acaster et al., J. Experimental Bot. 40:1121-1125 (1989)).

Acaster et al. have reported that the Kₘ values for ornithine ranged from 0.3 mM to 3.0 mM, and that the Kₘ values for carbamoyl phosphate ranged from 0.11 mM to 0.55 mM (Acaster et al., J. Experimental Bot. 40:1121-1125 (1989)). Reported molecular weights for ornithine carbamoyltransferase ranged from 148 to 167 kDa, and the subunit molecular weight for the enzyme from wild oat was reported as 38 kDa (Acaster et al., J. Experimental Bot. 40:1121-1125 (1989)). Ornithine carbamoyltransferase has also been isolated and characterized from the chloroplasts of leaf cells from Pisum sativum and compared with the enzyme partially purified from seedling shoots from this same plant species (DeRuiter et al., Plant Physiol. 77:695-699 (1985)). It was reported on the basis of this biochemical comparison that these tissues share a common form of ornithine carbamoyltransferase, with an approximate molecular weight of 77.6 kDa, a pH optimum of pH 8.3, and the following Kₘ values for substrates: ornithine, 1.2 mM; and carbamoyl phosphate, 0.2 mM.

Two isoenzymes of ornithine carbamoyltransferase with distinct biochemical and kinetic properties have been reported in sugarcane, pea seedlings, black alder root nodules, and apple leaves (Glenn et al., Plant Physiol. 60:122-126 (1977); Eid et al., Phytochem. 13:99-102 (1974); Martin et al., Acad. Sci. Paris Ser. 3:557-559 (1982); Spencer et al., Plant Physiol. 54:382-385 (1974)). Isolation and characterization of a cDNA encoding pea ornithine carbamoyltransferase has been reported (Williamson et al., Plant Mol. Biol. 31:1087-1092 (1996)). Southern blot analysis was utilized to detect the presence of multiple ornithine carbamoyltransferase genes in Pisum sativum. It has also been reported that the multiple isoenzymes of ornithine carbamoyltransferase in plants may serve different metabolic functions (Glenn et al., Plant Physiol. 60:122-126 (1977); Eid et al., Phytochem. 13:99-102 (1974)). The catabolic role of this enzyme in chloroplasts of Arabidopsis thaliana has been reviewed in Ludwig (Plant Physiol. 101:429-434 (1993)).

In addition to a reported gene sequence from pea, a cDNA sequence of ornithine carbamoyltransferase from Arabidopsis thaliana (GenBank Accession No. AJ002524, Quesada et al.), Medicago truncatula (GenBank Accession No. AA660661, Covitz et al.) and castor bean (van de Loo et al. Plant Physiol. 108:1141-1150 (1995)) have been reported.

Carbamoyl phosphate used in the ornithine carbamoyltransferase reaction is predominantly generated from glutamine, carbon dioxide, and ATP by the enzyme carbamoyl phosphate synthetase (EC 6.3.5.5). Carbamoyl phosphate synthetase also serves as a substrate in the pathway leading to de novo synthesis of pyrimidines (Jones, Annual Rev. Biochem. 49:253-279 (1980); Ross, The Biochem. of Plants, Vol. 6, Protein and Nucleic Acids, Marcus (ed.) Academic Press, New York, p. 169-205 (1981); Lovatt et al., Plant Physiol. 64:562-579 (1979); Mazus et al., Phytochem. 11:77-82 (1972)). The first step is catalyzed by aspartate carbamoyltransferase (EC 2.1.3.2), which has been reported to be localized to the chloroplasts in four plant species (Shibata et al., Plant Physiol. 80:126-129 (1986)). It has been reported that carbamoyl phosphate synthetase activity is feed back inhibited by pyrimidine nucleosides (e.g., UMP) and that this inhibition is relieved by ornithine (O'Neal et al., Biochem. Biophys. Res. Commun. 31:322-327 (1968)).

In contrast to most animal and fungi, which possess separate carbamoyl phosphate synthetase enzymes devoted to arginine and pyrimidine biosynthetic pathways, higher plants have one reported enzyme that is shared by both pathways (Makoff et al., Microbiol. Rev. (1978) 42:307-328; Rainer, Adv. Enzymol. Relat. Areas Mol. Biol. (1973) 39:1-90; O'Neal et al., Plant Physiol. 57:23-28 (1976); Ong et al., Biochem. J. 129:583-593 (1972)). It has been reported that in plants, carbamoyl phosphate synthetase is a regulatory enzyme that has a role in the metabolism of glutamine during seed development (O'Neal et al., Plant Physiol. 57:23-28 (1976); Ong et al., Biochem. J. 129:583-593 (1972); Kolloffel et al., Plant Physiol. 69:143-145 (1982)). Of note are the following findings: (1) arginine accumulates to a large degree in developing cotyledons; (2) nutrients supplied to the cotyledons are rich in glutamine; and (3) carbamoyl phosphate synthetase levels are 10-fold higher during early seed development than during germination (Flinn et al., Ann. Bot. 32:479-495 (1968); Lea et al., J. Exp. Bot. 30:529-537 (1979); Lewis et al., J. Exp. Bot 24:596-606 (1973); Millerd et al., Aust. J. Plant Physiol. 2:51-59 (1975); Pate et al., The Physiol. of the Garden Pea, Sutcliffe and Pate (eds.), Academic Press, London, p. 431-468 (1977); Kolloffel et al., Plant Physiol. 69:143-145 (1982)). Metabolic flux to arginine is also affected by the activity of ornithine carbamoyltransferase, since it is a determinant of the amount of carbamoyl phosphate that is committed to arginine biosynthesis as opposed to pyrimidine biosynthesis (Legrain et al., Eur. J. Biochem. 247:1046-1055 (1997)).

The reported penultimate reaction in the arginine biosynthetic pathway is catalyzed by argininosuccinate synthetase (EC 6.3.4.5). Argininosuccinate synthetase catalyzes the combination of citrulline and aspartate to form argininosuccinate using energy supplied by the conversion of ATP to AMP and PPi. Characterization of argininosuccinate synthetase from yeast and mammals reveals homotetramers with a subunit size of around 46 kDa (Van Vilet et al., Gene 95:99-104 (1990)). Pea cotyledons have been reported to possess high levels of argininosuccinate synthetase (Shargool et al., Canadian J. of Biochem. 47:467-475 (1969)). A regulatory role of argininosuccinate synthetase has been reported in that it is regulated by energy charge with arginine serving as a modifier of this regulation (Shargool, FEBS Letters 33:348 (1973)).

Argininosuccinate lyase (EC 4.3.2.1) catalyzes the final reported step in the biosynthetic pathway. Argininosuccinate lyase catalyzes the generation of arginine and fumarate from the catabolism of argininosuccinate (Davidson et al., Nature 169:313 (1952); Walker et al., J. Biol. Chem. 203:143 (1953); Buraczewski et al., Bull. Acad. Polon. Sci. Ser. Sci. Biol. 8:93 (1960); Rosenthal et al., Plant Physiol. (suppl.):xi (1966)). Argininosuccinate lyase was partially purified and characterized from cotyledons of germinating pea seeds (Shargool et al., Canadian J. Biochem. 46:393-399 (1968)). The pH optimum of argininosuccinate lyase was reported to be pH 7.9 and the Kₘ value for argininosuccinate was 0.2 mM. Studies of soybean cell cultures reported that argininosuccinate lyase activity declines after about 24 hours of growth (Shargool, Plant Physiol. 52:68-71 (1973)). The presence of a specific metal-dependent protease was reported to be responsible for the loss of argininosuccinate lyase activity in soybean cell cultures (Shargool, Plant Physiol. 55:632-635 (1975)). Argininosuccinate lyase has been reported as a regulatory step in the arginine pathway. An Arabidopsis thaliana cDNA encoding for argininosuccinate lyase has been reported (GenBank Accession No. Z97558, Hansen et al.).

Enzymes which catabolize arginine have been reported to be associated with nitrogen storage function in plants. Arginase (EC 3.5.3.1) catalyzes the breakdown of arginine into urea and ornithine. Levels of arginase activity increase in soybean axes during germination (Kang et al., Plant Physiol. 93:1230-1234 (1990)). It has been reported that arginase activities increase 10-fold in the seedlings of Arabidopsis thaliana during the first 6 days after germination (Zonia et al., Plant Physiol. 107:1097-1103 (1995)). The reported mole percentage of arginine is high in Arabidopsis thaliana storage protein relative to an “average” protein (Krumpelman et al., Plant Physiol. 107:1479-1480 (1995); VanEtten et al., J. Agric. Food Chem. 11:399-410 (1963); VanEtten et al., J. Agric. Food Chem. 11:399-410 (1963)).

Arginase has been purified to homogeneity from soybean axes and this enzyme was reported to be a 240 kDa multimeric protein with a subunit size of 60 kDa (Kang et al., Plant Physiol. 93:1230-1234 (1990)). Arginase has a reported optimum pH of 9.5, and a Kₘ value for arginine of 83 mM. A cDNA clone of the arginase gene from Arabidopsis thaliana has been reported (Krumpelman et al., Plant Physiol. 107:1479-1480 (1995)).

Another enzyme of arginine catabolism is arginine decarboxylase (EC 4.1.1.19), which catalyzes the conversion of arginine to agmatine. This reaction is the first reported step leading to the synthesis of polyamines from arginine (Tabor et al., Annu. Rev. Biochem. 53:749-790 (1984)). Plants can also utilize ornithine as a precursor to polyamine synthesis via the activity of ornithine decarboxylase. It has been reported that the arginine decarboxylase pathway is the predominant route for polyamine synthesis in non-dividing tissues, while the ornithine decarboxylase pathway is used predominantly in reproductive and dividing cells (Slocum et al., Arch. Biochem. Biophys. 235:283-303 (1984); Tabor et al., Annu. Rev. Biochem. 53:749-790 (1984); Evans et al., Annu. Rev. Plant Physiol. Plant Mol. Biol. 40:235-269 (1989); Pegg, Biochem. J. 234:249-262 (1986)). In addition, activity of arginine decarboxylase has been reported to increase during conditions of environmental stress, including salt stress and low oxygen levels (Flores et al., Cell. Mol. Biol. Plant Stress. eds. Key and Kosuge. A.R. Liss, Inc., New York. p. 93-114 (1985); Reggiani et al., Plant Cell Physiol. 31:489-494 (1990); Reggiani, Plant Cell Physiol. 35:1245-1249 (1994)). The existence of translational or post-transcriptional regulation of arginine decarboxylase activity in tomato plants and in osmotically stressed oat leaves spermine has been reported to inhibit processing of arginine decarboxylase to its mature form (Walden et al., Plant Physiol. 113:1009-1013 (1997); Borrell et al., Plant Physiol. 98:105-110 (1996); Rastogi et al., Plant Physiol. 103:829-834 (1993)).

Arginine decarboxylase has been isolated from numerous plant species, including oat seedlings, rice embryos, cucumber seedlings, and leaves of Vicia (Smith, Phytochem. 18:1447-1452 (1979); Vicente et al., Plant Cell Physiol. 22:1119-1123 (1981); Choudhuri et al., Agric. Biol. Chem. 46:739-743 (1982); Matsuda, Plant Cell Physiol. 25:523-530 (1984); Prasad et al., J. Biosci. 7:331-343 (1985)). Arginine decarboxylase purified from rice coleoptiles has been reported to have a molecular mass of 176 kDa and a subunit size of 63 kDa (Reggiani, Plant Cell Physiol. 35:1245-1249 (1994)). cDNA clones of arginine decarboxylase have also been reported for plant species, including oat, tomato, pea, and Arabidopsis thaliana (Bell et al., Mol. Gen. Genet. 224:431-436 (1990); Rastogi et al., J. Biol. Chem. 103:829-834 (1993); Perez-Amador et al., Plant Molecul. Biol. 28:997-1009 (1995); Malmberg et al., Plant Physiol. 111:S-21 (1996)).

Another enzyme which catalyzes the catabolism of arginine is arginine iminohydrolase (EC 3.5.3.6) which converts arginine to citrulline with the concomitant liberation of ammonia. It has been reported from studies on Arabidopsis thaliana that this chloroplastic enzyme represents the first reported step of a pathway (along with ornithine carbamoyltransferase and carbamate kinase) in which arginine is eventually broken down into ornithine, ammonium, bicarbonate, and ATP (Ludwig, Plant Physiol. 101:429-434 (1993)).

5. Proline Pathway

The proline pathway was first characterized in microorganisms using a combination of techniques including isotope competition, auxotrophic mutants, accumulation of intermediates in mutants and absence of enzymes in mutants (Vogel, In: Amino Acid Metabolism, McElroy and Glass eds., John Hopkins Press, Baltimore, Md., pp. 335-336 (1955)). The proline biosynthesis pathway in plants is reported to be homologous to the proline biosynthesis pathway in bacteria (Bryan, In: The Biochemistry of Plants; A Comprehensive Treatise, Miflin and Lea, eds., Academic Press, San Diego, Vol. 16, pp. 161-165, (1990); Leisinger, In: Escherichia coli and Salmonella typhimurium: Cellular and Molecular Biology, Neidhardt, Ingraham, Low, Magasanik, Schaechter, and Umbarger, American Society of Microbiology, Washington, pp. 345-351 (1987)). Unlike the bacterial proline pathway, the plant proline pathway can synthesize proline from glutamate and ornithine.

Stress-induced proline accumulation in plants and the status of the characterized genes and proteins associated with the proline pathway of plants has been reviewed by Hare and Cress, Plant Growth Regul. 21:79-102 (1997). Free proline accumulates in plants in response to biotic and abiotic stresses. Free proline has been reported to play a role in osmotic adjustment, subcellular structure stabilization, and free radical scavenging. Free proline is also associated with multiple cellular functions including but not limited to: reducing cellular acidification, priming oxidative respiration, maintaining NAD(P)⁺/NAD(P)H ratios, maintaining redox potential, enhancing the oxidative pentose phosphate pathway, providing precursors for nucleotide biosynthesis and secondary metabolites, enhancing nitrogen fixation, and providing an energy source for ADP phosphorylation.

It has been reported from radioisotope labeling studies that glutamate is the primary precursor for proline biosynthesis in stressed plant cells (Hu et al., Proc. Natl. Acad. Sci. 89:9354-9358 (1992)), whereas ornithine is reported to be utilized as a proline precursor in other metabolic processes. It has also been reported that the utilization of glutamate or ornithine for proline biosynthesis in plants may be dependent on the nitrogen status, developmental stage, and cell type (Hare and Cress, Plant Growth Regul. 21:79-102 (1997)).

The rate of proline accumulation in plant tissues is reported to be regulated by the rate of biosynthesis and degradation of proline (Kiyosue et al., Plant Cell 8:1323-1335 (1996)). Proline degradation has been reported to contribute carbon to the tricarboxylic acid cycle in energy intensive processes of thermogenic plants, nitrogen fixation, and in plants recovering from stress. In addition, proline degradation has been reported to contribute to the regulation of intracellular redox potential (Hare and Cress, Plant Growth Regul 21:79-102 (1997)).

Osmolytes (such as alcohols, sugars, proline, and glycine betaine) have been reported to accumulate in plants subjected to environmental stress, such as water deprivation and salinization. Free proline has also been reported to accumulate in plants subjected to environmental stress, such as water deprivation and salinization (Deluaney and Verma, Plant J. 4:215-223 (1993); Heuer, In: Handbook of Plant and Crop Stress, Pessarakli ed., Marcel Dekker, New York, pp. 363-381 (1994)), high temperature (Kuo and Chen, J. Am. Soc. Hort. Sci. 111:746-750 (1986)), low temperature (Naidu et al., Phytochem 30:407-409 (1991)), heavy metal toxicity (Alia et al., J. Plant Physiol. 138:554-558 (1991); Bassi and Sharma, Ann. Bot. 72:151-154 (1993)), pathogen infection (Seitz and Hoechester, Life Sci. 3:1033-1037 (1964); Labanauskas et al., J. Am. Soc. Hort. Sci. 99:497-500 (1974); Meon et al., Physiol. Plant Pathol. 12:251-256 (1978)), anaerobiosis (Aloni and Rosenshtein, Physiol. Plant 56:513-517 (1982)), nutrient deficiency (Goring and Thein, Biochem. Physiol. Pflanzen. 174:9-16 (1979); Vaucheret et al., Plant J. 2:559-569 (1992)), atmospheric pollution (Anbazhagan et al., J. Plant Physiol. 133:122-123 (1988)), and UV-irradiation (Pardha Saradhi et al., Biophys. Res. Commun. 209:1-5 (1995)).

i. Proline Biosynthesis Pathway

The bacterial proline biosynthesis pathway is reported to initiate with the phosphorylation of glutamate by γ-glutamyl kinase (proB gene) to form γ-glutamyl phosphate. Glutamic-γ-semialdehyde dehydrogenase (proA gene) converts γ-glutamyl phosphate to glutamic-γ-semialdehyde. Glutamic-γ-semialdehyde spontaneously cyclizes to Δ¹-pyrroline-5-carboxylate which is reduced to proline via Δ¹-pyrroline-5-carboxylate reductase (proC gene) (Leisinger, In: Escherichia coli and Salmonella typhimurium: Cellular and Molecular Biology, Neidhardt, Ingraham, Low, Magasanik, Schaechter, and Umbarger eds., American Society of Microbiology, Washington, pp. 345-351 (1987)). A similar pathway for proline synthesis has been reported for plants.

The first reported committed reaction of the plant proline biosynthesis pathway is catalyzed by the bifunctional enzyme Δ¹-pyrroline-5-carboxylate synthase (also referred to as delta-1-pyrroline-5-carboxylate synthase (EC 2.7.2.11 and EC 1.2.1.41)). Δ¹-Pyrroline-5-carboxylate synthase has been characterized in Vigna (Hu et al., Proc. Natl. Acad. Sci. (U.S.A.) 89:9345-9358 (1992)), and Arabidopsis (Savoure et al., FEBS Lett 372:13-19 (1995); Yoshiba et al., Plant J 7:751-760 (1995)). The reported plant enzymes are bifunctional and have two domains, a proB-like domain which exhibits γ-glutamyl kinase (proB) activity and a proA-like domain which exhibits glutamic-γ-semialdehyde dehydrogenase (proA) activity. Thus, in the first catalytic step, glutamate is converted into γ-glutamyl phosphate in an ATP dependent reaction. In the second catalytic step, γ-glutamyl phosphate is converted into glutamic-γ-semialdehyde in a NADPH dependent reaction. These two catalytic steps are performed by two enzymes (proA and proB) in the bacterial proline biosynthesis pathway. The plant Δ¹-pyrroline-5-carboxylate synthase activity is reported to have glutamic-γ-semialdehyde dehydrogenase-dependent γ-glutamyl kinase activity and to be feedback inhibited by proline. Glutamic-γ-semialdehyde spontaneously cyclizes to yield Δ¹-pyrroline-5-carboxylate.

Δ¹-Pyrroline-5-carboxylate reductase (also referred to as delta-1-pyrroline-5-carboxylate reductase (EC 1.5.1.2)) catalyses the conversion of Δ¹-pyrroline-5-carboxylate to proline in a NADPH dependent reaction. cDNA clones encoding Δ¹-pyrroline-5-carboxylate reductase activity have been isolated and characterized from pea (Williamson and Slocum, Plant Physiol 100:1464-1470 (1992)), soybean (Delauney and Verma, Mol. Gen. Genet. 22:299-305 (1990)), and Arabidopsis (Verbuggen et al., Plant Physiol 103:771-781 (1993)). In Arabidopsis, Δ¹-pyrroline-5-carboxylate reductase is reported to be cytosolic and the mRNA level is reported to be higher in roots and ripening seeds than in green tissues. Salt treatment of Arabidopsis plants is reported to increase the Δ¹-pyrroline-5-carboxylate reductase mRNA level five-fold. Thus, it has been suggested that the Δ¹-pyrroline-5-carboxylate reductase gene promoter region is subject to osmoregulation (Verbuggen et al., Plant Physiol 103:771-781 (1993)).

Plants also synthesize proline from ornithine via the transamination of ornithine to glutamic-γ-semialdehyde by ornithine δ-aminotransferase (also referred to as delta-aminotransferase (EC 2.6.1.13)). The enzyme ornithine δ-aminotransferase spontaneously cyclizes ornithine in the presence of 2-oxoglutarate through the intermediate glutamic-γ-semialdehyde to yield Δ¹-pyrroline-5-carboxylate. Δ¹-Pyrroline-5-carboxylate is then converted to proline by Δ¹-pyrroline-5-carboxylate reductase. The cDNA encoding ornithine 8-aminotransferase has been isolated from Vigna (Delauney and Verma, J. Biol. Chem. 268:18673-18678 (1993)). Enzymatic studies have been performed on ornithine 8-aminotransferase (Taylor and Stewart, Biochem. Biophys. Res. Commun. 101:1281-1289 (1981)). The ornithine δ-aminotransferase cDNA is reported to contain a mitochondrial targeting sequence. It has also been reported that transamination of ornithine occurs primarily in the mitochondria.

ii. Proline Degradation Pathway

Proline levels in plants is regulated, in part, by the rate of proline degradation. Proline is oxidized to Δ¹-pyrroline-5-carboxylate in plant mitochondria via proline dehydrogenase (oxidase) and Δ¹-pyrroline-5-carboxylate is converted to glutamate by Δ¹-pyrroline-5-carboxylate dehydrogenase (Elthon and Stewart, Plant Physiol 67:780-784 (1981)). In plants, both proline dehydrogenase and Δ¹-pyrroline-5-carboxylate dehydrogenase are reported to be bound to the matrix side of the inner mitochondrial membrane. Proline oxidation is reported to be involved in the transfer of electrons into the initial portion of the electron transport chain (Hare and Cress, Plant Growth Regul. 21:79-102 (1997)).

Plant proline dehydrogenase (oxygenase) (EC 1.4.3) is reported to be an oxygen-dependent flavoprotein localized in mitochondria (Elthon and Stewart, Plant Physiol. 67:780-784 (1981)). Proline dehydrogenase (oxygenase) converts proline to Δ¹-pyrroline-5-carboxylate in the presence of oxygen and FAD. A cDNA encoding a proline dehydrogenase (oxidase) has been isolated and characterized from Arabidopsis (Kiysue et al., Plant Cell 8:1323-1335 (1996)). It has been reported that proline dehydrogenase mRNA and protein accumulate in plant tissues in response to rehydration after dehydration and when plant tissue is incubated in the presence of high levels of proline (Yoshiba et al., Plant J 7:751-760 (1995)). It has also been reported that proline induction of proline dehydrogenase (oxidase) is inhibited by salt stress (Peng et al., Mol. Gen. Genet. 253:334-341(1996)).

Plant mitochondria are reported to contain two isoenzymes of Δ¹-pyrroline-5-carboxylate dehydrogenase (EC 1.5.1.12) (Forlani et al., Planta 202:242-248 (1997)). One isoform is reported to oxidize Δ¹-pyrroline-5-carboxylate from proline and the other isoform is reported to oxidize Δ¹-pyrroline-5-carboxylate from ornithine. The later isoenzyme is reported to form a complex with the mitochondrial ornithine δ-aminotransferase, thus channeling the substrates to degradation (Elthon and Stewart, Plant Physiol. 70:567-572 (1982)). Both isozymes convert Δ¹-pyrroline-5-carboxylate into glutamate in the presence of NADP+. A cDNA for the Δ¹-pyrroline-5-carboxylate dehydrogenase gene from the basidiomycete Agaricus has been reported (Schaap et al., Appl. Environ. Microbiol. 63:57-62 (1997)).

6. Glutamate/Glutamine and Aspartate/Asparagine Pathway

Primary nitrogen assimilation has been reviewed by Lam et al., Plant Cell 7:887-898 (1995); Brears and Coruzzi, Transgenic plants exhibiting enhanced nitrogen assimilation PCT WO 9509911. Nitrogen is often the rate-limiting element in plant growth and development. Agricultural crops often require supplementation with inorganic nitrogenous fertilizer to attain optimized crop yields. Since fertilizer is rapidly depleted from most soil types, it often has to be applied two or three times during the growing season. Nitrogenous fertilizers often account for 40% of the costs associated with crops such as corn and wheat.

Plants harvest nitrogen from their environment as inorganic compounds, namely nitrates and ammonia taken up from roots, and atmospheric nitrogen reduced to ammonia in nitrogen-fixing root nodules. Although small levels of ammonia and nitrate can be detected in vascular tissues (xylem and phloem), glutamine, glutamate and asparagine serve as predominant nitrogen-transport compounds and nitrogen donors in the biosynthesis of many plant compounds, including essentially all amino acids, nucleic acids, and other nitrogen-containing compounds, such as hormones and chlorophyll. Nitrogen may subsequently be channeled from glutamine and glutamate to aspartate or asparagine. The four nitrogen-transport amino acids generated by this pathway (glutamine, glutamate, aspartate, and asparagine) are the predominant amino acids found in most higher plants. In Arabidopsis thaliana, these amino acids represent 64% of the total amino acids found in a leaf extract. The amide amino acids glutamine and asparagine each carry an extra nitrogen atom in the amide group of their side chains and have been reported to play a role as nitrogen carriers in cellular metabolism.

It has been reported that asparagine is the predominant amino acid exported in phloem from leaves of dark-grown or dark-adapted plants. Glutamine is reported to be used primarily to transport assimilated nitrogen from roots to shoots. Glutamine and asparagine have other reported roles in plant metabolism besides being a nitrogen carrier. Glutamine is metabolically active in reactions that use an amide nitrogen atom. Asparagine is used for transport. Asparagine has not been reported to directly participate in nitrogen metabolism and is hydrolyzed to aspartate and ammonia by asparaginase (EC 3.5.1.1). Aspartate is a substrate utilized in the synthesis of proteins and amino acids.

Studies have reported that increased expression of the glutamate synthesizing enzymes correlates with increased storage protein metabolism (Osuji and Madu, Phytochemistry 39:495-503 (1995)). Illinois high protein maize lines (25% protein) show higher leaf glutamate dehydrogenase levels than those reported in Illinois low protein (5%) maize lines. Glutamine synthetase activity shows an inverse relationship between these two lines (Dembinski et al., Acta Physilogiae Plantarum 17:361-365 (1995)).

Inorganic nitrogen, in the form of nitrate, is taken up by plants and reduced to ammonia via the concerted actions of nitrate reductase (NR, EC 1.6.6.1) and nitrite reductase (NiR, EC 1.6.6.4). Atmospheric nitrogen can be reduced by the microbial enzyme nitrogenase (N₂ase, EC 1.18.6.1). Ammonia is then assimilated into glutamine and glutamate through the combined actions of glutamine synthetase (GS, EC 6.3.1.2) and ferredoxin-dependent glutamate synthase (Fd-GOGAT, EC 1.4.7.1) or NADH-dependent glutamate synthase (NADH-GOGAT, EC 1.4.1.14). Glutamate dehydrogenases (GDH; EC 1.4.1.2, EC 1.4.1.3, and EC 1.4.1.4) are reported to be the primary route of nitrogen assimilation in microorganisms under certain in vitro conditions. GDH in higher plants is reported to function largely in glutamate catabolism. Nitrogen may subsequently be channeled from glutamine and glutamate to aspartate by aspartate aminotransferase (AsAT; EC 2.6.1.1) or to asparagine by asparagine synthetase (AS; also referred to as asparagine synthase EC 6.3.5.4).

Primary nitrogen assimilation has been reviewed by Lam et al., Plant Cell 7:887-898 (1995) and Oaks, Can. J. Bot. 72:739-750 (1995). Light and metabolic status are two signals that govern the regulation of amide amino acid metabolism. Regulation by light of asparagine synthetase, glutamine synthetase and ferredoxin-glutamate synthase is an example of reciprocal gene regulation. Light up-regulates glutamine synthetase and ferredoxin dependent glutamate synthase expression and down-regulates asparagine synthetase expression.

Physiological changes in expression of these genes are reflected in corresponding changes in the amino acid profiles of Arabidopsis thaliana leaf extracts. For example, asparagine levels are high in the dark and glutamine levels are high in the light. During the light period, when photosynthesis occurs and carbon skeletons are abundant, nitrogen is assimilated and transported as glutamine. Levels of mRNA for genes associated with glutamate and glutamine synthesis are induced by both light and sucrose. Light represses the synthesis of asparagine, which accumulates in tissues of dark adapted plants. Levels of asparagine synthetase mRNA are induced in dark adapted plants. Induction of asparagine synthetase mRNA is repressed by light or high sucrose.

Under conditions of carbon limitation or nitrogen excess, plants activate genes associated with asparagine biosynthesis. Certain nitrogen assimilation gene promoters, including those of pea GS2 and AS, are reported to contain light responsive elements.

Nitrate and ammonia have been reported to regulate genes involved in the primary assimilation of nitrogen. In maize leaves GOGAT, nitrate reductase and nitrite reductase levels have been reported to respond to nitrate levels. In roots, there is up regulation of nitrate reductase, nitrite reductase, glutamine synthetase (both cytosolic and plastidic GS's), ferredoxin- and NADH-glutamate synthase in response to nitrate. Glutamine synthetase, ferredoxin-glutamate synthase, NADH glutamate synthase, glutamate dehydrogenase, and PEP carboxylase levels are up regulated in response to ammonia. Gowri et al., Plant Mol. Biol. 18:55-64 (1992), have reported that the production of nitrate reductase mRNA is not affected by cyclohexamide treatment and that the gene is turned on in response to nitrate levels.

Nitrogen metabolism enzymes play a role in nitrogen assimilation. Nitrogen assimilation takes place primarily in the chloroplast where most of the nitrate is converted to ammonia. Ammonia is in turn converted to glutamine and glutamate by the action of GS and GOGAT. Ammonia generated during the photorespiratory process or during the deamination of amino acids is reassimilated.

NADPH dependent glutamate synthase (EC 1.4.1.13) catalyzes the conversion of glutamine and 2-oxoglutarate to yield two molecules of glutamate. NADPH dependent glutamate synthase can use either NADH or NADPH as a coenzyme (Lea et al., In: Ammonia assimilation in higher plants: Nitrogen Metabolism of plants, Mengel and Pilbeam, eds., 153-186 (1992)). It has also been reported that the NADH-dependent form dominates in higher plant tissue. The NADPH-dependent form is found primarily in microbes and lower plants.

Properties and a general description of NADH dependent glutamate synthase have been reviewed by Lea, In: U.K. Plant Biochem, 273-306 (1997). NADH dependent glutamate synthase (EC 1.4.1.14) catalyzes conversion of glutamine and 2-oxoglutarate to two molecules of glutamate. In green leaves the activity of this NADH dependent glutamate synthase enzyme is lower than the ferredoxin-dependent glutamate synthase enzyme activity. NADH dependent glutamate synthase activity has also been reported in a variety of non-green tissues such as roots, cotyledons, and tissue cultured cells and this enzyme has been reported to play a role in the ammonia assimilation in nitrogen fixing nodules. This enzyme is a monomer and has a reported molecular weight in the region of 200-225 kDa.

Properties and a general description of ferredoxin dependent glutamate synthase (EC 1.4.7.1) have been reviewed by Lea, In: U.K Plant Biochem 273-306 (1997). This enzyme catalyzes the conversion of glutamine and 2-oxoglutarate to yield two molecules of glutamate. Ferredoxin dependent glutamate synthase was first reported in pea leaves, is an iron-sulfur flavoprotein and can represent up to 1% of the protein content of leaves. Ferredoxin dependent glutamate synthase is a monomeric protein with a molecular weight of 140-160 kDa. Tissue fractionation studies have shown that ferredoxin dependent glutamate synthase is localized in the chloroplast of the leaf. Activity of ferredoxin dependent glutamate synthase increases during leaf development in the light. In maize, the transcription level of ferredoxin dependent glutamate synthase has been reported to increase after illumination of etiolated leaves. Similar results were obtained in tobacco. In tomato, it has been reported that a similar response was mediated by phytochrome.

Aspartate aminotransferase has been reviewed by Lea, In: U.K. Plant Biochem. 273-306 (1997). Aspartate aminotransferase catalyzes the transfer of an amino group from the 2 position of glutamate, which generates aspartate and alpha-ketoglutarate. Glutamate is an amino donor for the reaction (Glutamate+Oxaloacetate←→2-Oxoglutarate+Asparate). Aspartate aminotransferase plays a role in the formation of aspartate required for the synthesis of the proteins and the aspartate family of amino acids. Additionally, aspartate aminotransferase has three other reported roles. The first role of aspartate aminotransferase is in the transfer of amino groups from glutamate via aspartate to asparagine in nitrogen fixing root nodules. 2-Oxoglutarate molecules are reported to cycle in a manner that collects additional amino groups in the glutamate synthase reaction. In addition, oxaloacetate is synthesized in a manner that is catalyzed by the action of PEP carboxylase. The second reported role of aspartate aminotransferase is in the malate-oxaloacetate shuttle, which transfers reducing power from mitochondria and chloroplasts to the cytoplasm via the enzyme malate dehydrogenase. Due to the inherent instablility of oxaloacetate, it is also transaminated to asparate to facilitate transport. The third role of aspartate aminotransferase is in the formation of oxaloacetate. In conjunction with the PEP carboxylase reaction in C4 plants, aspartate is transported from mesophyll cells to bundle sheath cells in NAD-ME type plants. Distinct isoenzymes of aspartate aminotransferase have been reported in the mitochondria, chloroplast, peroxisomes, and cytoplasm of higher plants and reports based on molecular analysis of this gene in plants have illustrated that this gene can be present as a multigene family.

Alanine aminotransferase (EC 2.6.1.2) has been reviewed by Lea, In: U.K. Plant Biochem., 273-306 (1997). Alanine aminotransferase catalyzes the transfer of the amino group from the 2 position of the amino acid to yield an oxo acid and amino acid. Glutamate is an amino donor for alanine aminotransferase reaction (Glutamate+Pyruvate←→2-Oxoglutarate+Alanine). Alanine aminotransferase liberates 2-oxoglutarate, which returns to the glutamate synthase cycle. Alanine aminotransferases have been detected in plants that can synthesize all amino acids found in plant proteins, excluding proline, when the corresponding 2-oxo acid precursor is present. Nitrogen can be distributed from glutamate, via asparate and alanine to all such amino acids. Reversibility of the reaction has been reported to allow for sudden changes in demand for key amino acids.

Glutamine synthetase (GS, EC 6.3.1.2) has been reviewed by Lea, In: U.K. Plant Biochem. 273-306 (1997). It has been reported that in higher plants that glutamine synthetase (GS) is a port of entry into amino acids. GS catalyzes an ATP-dependent conversion of glutamate into glutamine (Glutamate+Ammonia+ATP→Glutamine+AMP+Pi). GS is an octameric protein with a native molecular weight of 350-400 kDa and has an affinity for ammonia. GS isozymes have been reported in Phaseolus vulgaris, and Pisum sativum. Five genes coding for GS have been reported in Phaseolus vulgaris. Gln-alpha, gln-beta, and gln-gamma genes encode cytosolic alpha, beta, and gamma polypeptides, which are located in the cytoplasm. A gln-delta gene that encodes a chloroplastic form and a gln-epsilon gene have also been reported. Chloroplastic polypeptides assemble into octamers of identical subunits. Cytosolic polypeptides assemble into a range of isoenzymes containing various proportions of alpha, beta, and gamma polypeptides. It has been reported that Pisum Sativum has three cytosolic genes and one chloroplastic GS gene.

Properties of glutamate dehydrogenase (GDH; EC 1.4.1.2, EC 1.4.1.3, and EC 1.4.1.4) has been reviewed by Lam et al., Plant Cell 7:887-898 (1995). In vitro studies have reported that a plant GDH can catalyze two distinct biochemical reactions: the amination of alpha-ketoglutarate and the deamination of glutamate. The majority of higher plant GDH enzymes characterized to date have a high Kₘ for ammonia and have been reported to play a catabolic role. In some cases, an ammonia assimilation role for GDH has been reported. Another reported role for GDH is in the ammonia detoxification process. It has also been reported that GDH assimilates a portion of the photorespiratory ammonia necessary to generate catalytic amounts of glutamate for the GS/GOGAT cycle.

Gamma glutamylcyclotransferase (E.C. 2.3.2.4) catalyzes the conversion of epsilon-(L-gamma-glutamyl)-L-lysine to lysine and 5-oxo-L-proline (Fink and Folk, Mol. Cell. Biochem. 38:59-67 (1981); Steinkamp et al., Fed. Rep. Ger. Physiol. Plant. 69:499-503 (1987)).

5-Oxoprolinase catalyzes the ATP-dependent decyclization of 5-oxo-L-proline to L-glutamate (Li et al., J. Biol. Chem. 264:3096-3101 (1989)). 5-Oxoproloinase in tobacco grown with glutathione as the sole sulfur source catalyzes the conversion of 5-oxoproline to glutarnic acid (Rennenberg et al., Z. Naturforsch 35:708-711 (1980)).

Asparagine synthetase (AS, EC 6.3.5.4) has been reviewed by Lea, In: U.K. Plant Biochem, 273-306 (1997). Asparagine synthetase catalyzes the transfer of an amide group of glutamine to aspartate (Glutamine+Aspartate+ATP→Asparagine+Glutamate+ADP+Ppi). It has been reported that the cotyledons of germinating seeds are a source of asparagine synthetase activity. Asparagine synthetase has been studied in, for example, lupins and soybeans. Asparagine synthetase is also able to use ammonia as a substrate in maize roots. Asparagine synthetase also plays a physiological role in root nodules. Two classes of asparagine synthetase cDNAs, AS1 and AS2, have been reported from pea. These classes encode homologues that are distinct polypeptides. AS1 and AS2 have molecular weights of 66.3 kDa and 65.6 kDa respectively. Additionally, glutamine binding sites were detected at the amino acid terminus of both AS1 and AS2. It has been reported that the level of the AS1 mRNA is increased in leaves of both etiolated seedlings and mature pea plants in the dark. Light repression of AS1 mRNA synthesis was reported to be phytochrome mediated. It has also been reported that both AS1 and AS2 mRNA accumulate in germinating cotyledons and nitrogen fixing nodules.

Glutaminase (EC 3.5.1.2) hydrolyzes glutamine to form glutamate (Voet and Voet, In: Biochemistry, John Wiley & Sons, New York, 690-691 (1990); Duran et al., Microbiol. 141:2883-2889 (1995)). It has been reported that glutaminase participates in a glutamine cycle in which it degrades glutamine that can be resynthesized by glutamine synthetase. Glutaminase activity has been further reported by Bigot and Boucaud, Phytochemistry 31:4071-4074 (1992).

1-Pyrroline-5-carboxylate dehydrogenase (EC 1.5.1.12) catalyzes the first two reported steps in the biosynthesis of proline in plants (Zhang et al., J. Biol. Chem. 270:20491-20496 (1995). 1-Pyrroline-5-carboxlyate dehydrogenase catalyzes the conversion of proline to glutamic acid (Yoshiba et al., Plant Cell Physiol. 38:1095-1102 (1997)). Yoshiba et al., Plant Cell Physiol. 38:1095-1102, have also reported that such metabolism of proline is inhibited when proline accumulates during dehydration and is activated when rehydration occurs.

NADP⁺ dependent isocitrate dehydrogenase (ICDH, EC 1.1.1.42) has been reviewed by Fieuw et al., Plant Physiol. 107:905-913 (1995). NADP⁺ dependent isocitrate dehydrogenase catalyzes the reversible conversion of isocitrate to 2-oxoglutarate. Under in vitro conditions, the equilibrium of this reaction is dependent on the pH of the assay solution. For etiolated pea seedlings the optimal pH of the forward and reverse reactions were reported to be pH 8.4 and pH 6.0, respectively. NADP⁺ dependent isocitrate dehydrogenase is stable between pH 7.0 and pH 8.0. It has been reported that NADP⁺ dependent isocitrate dehydrogenase has been found in prokaryotes and located in different compartments of eukaryotes such as the cytosol, mitochondria, and peroxisomes. About 90% of NADP⁺ dependent isocitrate dehydrogenase activity is located in the cytosol, 10% is located in chloroplasts, less than 1% is present in the peroxisomes, and less than 1% is found in mitochondria NADP⁺ dependent isocitrate dehydrogenase exhibits a requirement for divalent metal ions such as Mn²⁺ or Mg²⁺.

In Escherichia coli, isocitrate dehydrogenase is reported to be a Krebs cycle enzyme, regulated via reversible phosphorylation, depending upon growth conditions. For Salmonella typhimurium and the protozoan Crithidia fasciculata, a regulation of isocitrate dehydrogenase by cellular ATP and 2-oxoglutatrate has been reported. In higher plants, NADP⁺ dependent isocitrate dehydrogenase is inhibited by glyoxylate and oxaloacetate and NADPH and 2-oxoglutarate. Citrate is a competitive inhibitor of NADP⁺ dependent isocitrate dehydrogenase activity. Additionally, it has been reported that the activity of NADP⁺ dependent NADP⁺ dependent isocitrate dehydrogenase may also be controlled by the intracellular NADPH/NADP⁺ ratio.

Glutamine has been reported to be a positive effector of isocitrate dehydrogenase. Similar to the protozoan and animal system NADP⁺ dependent isocitrate dehydrogenase of higher plants has also been reported to play a role in replenishing the cytosol with reducing power (i.e., NADPH), particularly during metabolic limitation of the pentose phosphate pathway. NADP⁺ dependent isocitrate dehydrogenase has also been reported to play a role in supplying carbon skeletons for NH₃ assimilation. 2-Oxoglutarate has been reported to function as a metabolite, linking nitrogen and carbon metabolism.

Glutamate decarboxylase (GAD, EC 4.1.1.15) has been reported by Baum et al., EMBO 15:2988-2996 (1996). Glutamate decarboxylase (GAD) catalyzes the decarboxylation of glutamate, yielding CO₂ and gamma-aminobutyrate (GABA) (Baum et al., EMBO 15:2988-2996 (1996)). GABA is a ubiquitous non-protein amino acid and neurotransmitter inhibitor in certain organisms. It has been reported that plants possess a form of GAD that binds calmodulin (CaM) (Arazi et al., Plant Physiol. 108:551-561 (1995)). Reported regulation of GAD by Ca²⁺/CaM in plants has been reported to reflect the requirement for rapid GAD modulation in response to external signals. GAD activity is reported to be stimulated by stresses such as hypoxia, temperature shock, water stress and mechanical manipulation. It has been further reported that GAD plays a role in plant development as GAD's expression in leaves, flowers, and germinating seeds is developmentally regulated by transcriptional and/or post-transcriptional processes.

Succinate-semialdehyde dehydrogenase (EC 1.2.1.24) catalyzes the irreversible reaction in which succinate-semialdehyde is oxidized to succinate. This process reduces one molecule of NAD. Most studies have concerned animal and microbial systems. Succinate-semialdehyde dehydrogenase activity in potato tuber has been reported by Narayan et al., Indian Arch. Biochem. Biophys. 275:469-477 (1989). Succinate-semialdehyde dehydrogenase has also been reported to have a native molecular weight of 145,000 kDa and under denaturing conditions has a polypeptide band of 35,000 kDa (SDS-Page). It has also been reported that succinate-semialdehyde dehydrogenase exhibits a specificity for succinate-semialdehyde and NAD and requires a thiol compound for maximal activity.

4-Aminobutyrate (GABA) aminotransferase (EC 2.6.1.19) has been described by Givan, In: Aminotransferases in Higher Plants, eds. Stumpf and Conn, 329-355 (1980) and Brown et al., Plant Physiol. 115:1-5 (1997). 4-Aminobutyrate (GABA) aminotransferase catalyzes the reversible reaction of GABA and pyruvate to succinate-semialdehyde and alanine. It has been reported that peanut mitochondria 4-aminobutyrate (GABA) aminotransferase has a substrate preference for pyruvate which is about five times greater than that of oxoglutarate. A substrate preference of pyruvate over oxoglutarate was also reported in radish leaf extracts. Evidence for GABA-pyruvate and GABA-oxoglutarate has been reported. An oxoglutarate-dependent enzyme with an affinity for GABA has been reported. In root nodule tissue, GABA transamination has been reported to take place using 2-oxoglutarate as the amino acceptor. Use of 2-oxoglutarate has been reported to be associated with rhizobial bacteriod symbionts. A GABA transaminase isolated from mushroom was reported to be highly specific for 2-oxoglutarate. Delta-aminovalerate was an alternative donor substrate for mushroom for 2-oxoglutarate.

N-Acetylglucosamine kinase (EC 2.7.1.59) catalyzes the phosphorylation of N-acetylglucosamine (Allen and Walker, Biochem. J. 185:565-575 (1980)). It has been reported that N-acetylglucosamine kinase is a symmetrical dimer of mol. wt. 80,000. Allen and Walker, Biochem. J. 185:577-582 (1980), have reported that N-acetyl-D-glucosamine inhibits the phosphorylation of D-glucose.

D. Plant Hormones Pathways and Other Regulatory Molecules

1. Cytokinin Pathway

Plant hormones, produced in response to genetic, environmental or chemical stimuli (Goldberg, Science 240:1460-1467 (1988); Letham, In: Phytohormones and Related Compounds—A Comprehensive Treatise, eds. Letham et al., Amsterdam, Elsevier North Holland. 1:205-263 (1978); von Sachs, Arb. Bot. Inst. Wurzburg 2:452-488 (1880)), play a role in controlling the growth, development and environmental responses of plants.

Cytokinins are a class of plant hormones with a structure resembling adenine. Cytokinins, in combination with auxin, promote cell division. Cytokinins are associated with many aspects of plant growth and development (Horgan, Advanced Plant Physiology, ed. Wilkins, Pitman, London:90-116 (1984); Skoog et al., Biochemical Actions of Hormones, ed. Litwack, Academic Press, London, Vol. VI:335-413 (1979)). Cytokinins have been reported in almost all higher plants as well as mosses, fungi, and bacteria. In addition to occurring in higher plants as free compounds, cytokinins may also occur as component nucleosides in tRNA of plants, animals, and microorganisms.

Kinetin, the first cytokinin to be discovered, was so named because of its ability to promote cytokinesis (cell division). Although kinetin is a natural compound, it is not made in plants, and is therefore usually considered a “synthetic” cytokinin. Two common forms of cytokinin in plants are zeatin and zeatin riboside (maize) (Letham, Life Sci. 2:569-573 (1963)). More than 200 known natural and synthetic cytokinins have been reported.

Several cytokinin related mutations have also been reported. For example, the ckr1 mutant of Arabidopsis is resistant to the cytokinin bezyladenine (Su and Howell, Plant Physiol. 99:1569-1574 (1992)). The Arabidopsis mutant amp1 has been reported to be a negative regulator of cytokinin biosynthesis (Chadbury et al., Plant J. 4:907-916 (1993)).

Cytokinin concentrations are highest in meristematic regions and areas of continuous growth potential such as roots, young leaves, developing fruits, and seeds (Arteca, Plant Growth Substances: Principles and Applications, eds. Chapman & Hall, New York (1996); Mauseth, Botany: An Introduction to Plant Biology, ed. Saunders, Philadelphia: 348-415 (1991); Raven et al., Biology of Plants, ed. Worth, New York: 545-572 (1992); Salisbury and Ross, Plant Physiology, ed. Wadsworth, Belmont, Calif.: 357-407, 531-548 (1992)).

It has been reported that the induced cytokinin response varies depending on the type of cytokinin and plant species (Davies, Plant Hormones: Physiology, Biochemistry and Molecular Biology, Kluwer, Dordrecht (1995); Mauseth, Botany: An Introduction to Plant Biology, Saunders, Philadelphia: 348-415 (1991); Raven et al., Biology of Plants, ed. Worth, New York: 545-572 (1992); Salisbury and Ross, Plant Physiology, ed. Wadsworth, Belmont, Calif.: 357-407, 531-548 (1992)). Elevated cytokinin levels are associated with the development of seeds in higher plants, and have been demonstrated to coincide with maximal mitotic activity in the endosperm of developing maize kernels, cereal grains, and fruits. Exogenous cytokinin application (via stem injection) has been shown to directly correlate with increased kernel yield in maize. In addition, plant cells transformed with the ipt gene from Agrobacterium tumefaciens showed increased growth corresponding to an increase in endogenous cytokinin levels upon induction of the enzyme. Cytokinins have been reported to confer thermotolerance in certain physiological processes such as plastid biogenesis and endosperm cell division (Cheikh and Jones, Plant Physiol. 106:45-51 (1994); Parthier, Biochem. Physiol Pflanz 174:173-214 (1979); Jones et al., Crop Science 25:830-834 (1985)).

Reviews of cytokinin metabolism, compartmentalization, conjugation and cytokinin metabolic enzymes have been presented by Jameson, Cytokinins, eds. Mok and Mok, Boca Raton, Fla., 113-128 (1994); Letham and Palni, Ann. Rev. Plant Physiol. 34:163-197 (1983); McGaw et al., In: Biosynthesis and metabolism of plant hormones, Soc. Exp. Biol. Seminar Series, eds. Crozier and Hillman, Cambridge University Press, Cambridge, Vol. 23, Chapter 5 (1984); McGaw and Horgan, Biol. Plant 27:180 (1985); McGaw et al., In: Plant Hormones: Physiology, Biochemistry and Molecular Biology, ed. Davies, Kluwer, Dordrecht, 98-117 (1995); Mok and Martin, Cytokinins, eds. Mok and Mok, Boca Raton, Fla., 129-137 (1994); Salisbury and Ross, Plant Physiology, Belmont, Calif.: ed. Wadsworth, 357-407, 531-548 (1992).

i. Biosynthesis of Cytokinins

Cytokinins are generally found in higher concentrations in meristematic regions and growing tissues. It has been reported that cytokinins are synthesized in the roots and translocated via the xylem to the meristematic regions and growing shoots of the plant. Although cytokinin biosynthesis in developed plants takes place mainly in roots (Engelbrecht, Biochem. Physiol. Pflanzen 163:335-343 (1972); Henson et al., J. Exp. Bot 27:1268-1278 (1976); Sossountzov et al., Planta 175:291-304 (1988); Van Staden et al., Ann. Bot. 42:751-753 (1978)), smaller amounts can be synthesized by the shoot apex and some other plant tissues.

The level of active cytokinin at a particular site of action has been reported to be influenced by a large number of factors: de novo synthesis; oxidative degradation; reduction; formation and hydrolysis of inactive conjugates; transport into and out of particular cells; subcellular compartmentalization to or away from sites of action. It has also been reported that physiological responses may be modulated by variations in the ability of cells to respond to a particular concentration of free cytokinin.

Cytokinin biosynthesis occurs through the biochemical modification of adenine (McGaw et al., In: Plant Hormones: Physiology, Biochemistry and Molecular Biology, ed. Davies, Kluwer, Dordrecht: 98-117 (1995); Salisbury and Ross, Plant Physiology, Belmont, Calif.: ed. Wadsworth, 357-407, 531-548 (1992)). Plants appear to synthesize cytokinins either directly by addition of isopentenylpyrophosphate to AMP by an adenylate:isopentenyltransferase (cytokinin synthase) producing isopentenyladenosine 5′ phosphate (“[9R-5′P]iP”), which in turn serves as an intermediate for further modifications, or indirectly via isopentenylation of adenosine residues of tRNA by tRNA:isopentenyltransferase (McGaw et al., In: Plant Hormones: Physiology, Biochemistry and Molecular Biology, ed. Davies, Kluwer, Dordrecht: 98-117 (1995)). [9R-5′P]iP may be modified by dephosphorylation, deribosylation, hydroxylation and reduction to produce a variety of derivatives with potential activity (Binns, Annu. Rev. Plant Physiol. Plant Mol. Biol. 45:173-196 (1994)). Further, conjugation may modulate levels of active cytokinins (Letham and Palni, Ann. Rev. Plant Physiol. 34:163-197 (1983)).

In the biosynthesis of tRNA cytokinins, mevalonic acid pyrophosphate undergoes decarboxylation, dehydration and isomerization to yield 2-isopentyl pyrophosphate (“iPP”). iPP then condenses with the relevant adenosine residue in the tRNA to give the N⁶(Δ²-isopentenyl)adenosine (“[9R]iP”) moiety. With the exception of [9R]iP and to a lessor extent cis- and trans-[9R]Z, the free and tRNA cytokinins are structurally distinct (e.g., free Zeatin (“Z”) is mainly the trans isomer (trans-Zeatin while Z present in tRNA is mainly the cis isomer (McGaw et al., In: Plant Hormones: Physiology, Biochemistry and Molecular Biology, ed. Davies, Kluwer, Dordrecht, 98-117 (1995).

The de novo biosynthesis pathway of cytokinins in plants includes the following enzymes: isopentyltransferase, 5′-nucleosidase, adenine nucleotidase, adenine phosphorylase, adenine kinase, adenine phosphoribosyl transferase, microsomal mixed function oxidases, Zeatin reductase, O-glucosyltransferase, O-xylosyltransferase, β-(9-cytokinin-alamino)synthase, cytokinin oxidase, β-glucosidase, and Zeatin cis-trans isomerase.

Isopentyltransferase catalyzes the first reaction of the pathway in which N⁶(Δ²-isopentenyl)adenosine-5′-monophosphate (“[9R-5′P]iP”) is generated from iPP and AMP.

5′-nucleotidase catalyzes the conversion of [9R-5′P]iP to [9R]iP. The reaction catalyzed by the enzyme 5′-nucleotidase has been reported in wheat germ extract (Chen et al., Plant Physiol. 67:494-498 (1981); Chen et al., Plant Physiol. 68:1020-1023 (1981)) and in tomato leaf and root extracts (Burch and Stuchbury, Phytochemistry 25:2445-2449 (1986); Burch and Stuchbury, J. Plant Physiol. 125:267-273 (1986)). Adenine kinase catalyzes the reversion of [9R]iP to [9R-5′P]iP. Alternatively, [9R-5′P]iP can be converted to t-Zeatin riboside-5′-monophosphate (“[9R-5′P]Z”) by a microsomal mixed function oxidase.

Adenosine nucleotidase catalyzes the conversion of [9R]iP to iP. This reaction can be reversed by the enzyme adenine phosphorylase. Alternatively, [9R]iP can be converted to t-Zeatin riboside (“[9R]Z”) by a microsomal mixed function oxidase. Under another reaction mechanism, adenosine can be cleaved from [9R]iP by cytokinin oxidase. The enzyme adenine phosphoribosyl transferase can catalyze the conversion of iP to [9R-5′P]iP. Adenine phosphoribosyl transferase which is one of the salvage routes in plants for converting adenosine to AMP has also been shown to catalyze the phosphoribolyzation of cytokinin bases from a number of plant sources, including wheat germ (Chen et al., Arch. Biochem. Biophys. 214:634-641 (1982)), tomato (Burch et al., Physiol. Plant 69:283-288 (1987)), A. thaliana (Moffatt et al., Plant Physiol. 95:900-908 (1991)) and Acer psuedoplatanus (Doree and Guern, Biochem. Biophys. Acta 304:611-622 (1973); Sadorge et al., Physiol. Veg. 8:499-514 (1970)).

The cytokinins N⁶(Δ²-isopentenyl)adenosine-7-glucoside (“[7G]iP”) and N⁶(Δ²-isopentenyl)adenosine-9-glucoside (“[9G]iP”) are generated from iP from the enzymes Zeatin reductase and O-glucosyltransferase (such as cytokinin-9-glucosyl transferase), respectively. Under another reaction mechanism, adenine can be cleaved from iP by cytokinin oxidase.

In addition to converting [9R-5′P]iP to [9R]iP, 5′-nucleotidase can also catalyze the conversion of [9R-5′P]Z to [9R]Z. Adenine kinase can catalyze the conversion of [9R]Z to [9R-5′P]Z.

O-glucosyltransferase catalyzes the conversion of [9R]Z to t-Zeatin riboside-O-glucoside (“(OG)[9R]Z”). O-glucosyltransferase can also remove the glucoside group from (OG)[9R]Z to regenerate [9R]Z. Adenosine can be cleaved from [9R]Z by cytokinin oxidase. Alternatively, adenine nucleotidase can convert [9R]Z to Z. Adenine phosphorylase can catalyze the conversion of Z back into [9R]Z.

The cytokinins dihidroZeatin (“(diH)Z”), Zeatin-7-glucoside ([7G]Z), Zeatin-9-glucoside (“[9G]Z”), and lupinic acid (“[9Ala]Z”) are generated from Z by the enzymes Zeatin reductase, O-glucosyltransferase, Zeatin reductase and β-(9-cytokinin alamino) synthase, respectively. Zeatin cis-trans isomerase catalyzes the isomerization of Zeatin between its cis and trans isomers. O-glucosyltransferase catalyzes the addition of a glucoside residue to Z to form t-Zeatin-O-glucoside (“(OG)Z”) or removal of a glucoside residue from (OG)Z to form Z.

The cytokinins dihydroZeatin-9-glucoside (“(diH)[9G]Z”), dihydroZeatin-7-glucoside (“(diH)[7G]Z”), and dihydrolupinic acid (“(diH)[9Ala]Z”) are generated from (diH)Z by the enzymes β-(9-cytokinin alamino)synthase, Zeatin reductase, and O-glucosyltransferase, respectively. O-glucosyltransferase catalyzes the addition of a glucoside residue to (diH)Z to form t-Zeatin-O-glucoside (“(diHOG)Z”) or removal of a glucoside residue from (diHOG)Z to form (diH)Z. Alternatively, (diH)Z can be converted into dihydrozeatin riboside ((diH)[9R]Z) by adenine phosphorylase. The enzyme adenine nucleotidase can catalyze the conversion of (diH)[9R]Z to (diH)Z.

O-glucosyltransferase catalyzes the addition of a glucoside residue to (diH)[9R]Z to form t-dihydroZeatin riboside-O-glucoside (“(diHOG)[9R]Z”) or the removal of a glucoside residue from (diHOG)[9R]Z to form (diH)[9R]Z. The cytokinin dihydroZeatin riboside-5′-monophosphate (“(diH)[9R-5′P]Z”) is generated from (diH)[9R]Z by the enzyme adenine kinase. This reaction can be reversed by the enzyme 5′-nucleotidase.

It is understood that the above description of the de novo biosynthesis of cytokinins only describes the core of the biosynthesis pathway. Other enzymes have been reported to be involved in this pathway.

Active cytokinins can be inactivated by degradation or conjugation to different low-molecular-weight metabolites, such as sugars and amino acids. The enzyme cytokinin oxidase plays a role in the degradation of cytokinins. This enzyme removes the side chain and releases adenine, the backbone of all cytokinins. Cytokinin oxidases are reported to remove cytokinins from plant cells after cell division. Cytokinin derivatives are also made.

β-glucosidase (EC 3.2.1.21) has been reported to cleave the biologically inactive hormone conjugates of cytokinin-O-glucoside to release the active cytokinin (Brzobohaty et al., Science 262:1051-1054 (1993); Campos et al., Plant J. 2:675-684 (1992)). β-glucosidase catalyzes the hydrolysis of aryl and alkyl β-D-glucosides and/or cellobiose with the release of β-D-glucose (Reese, Recent Adv. Phytochem. 11:311 (1977)). The enzyme has been purified from maize and has a molecular weight of 60 kD (Esen, Plant Physiol. 98:174-182 (1992); Esen et al., Biochem. Genet. 28:319-336 (1990)). Esen et al. have identified the rolC gene of Agrobacterium rhizogenes which encodes for a cytokinin β-glucosidase and which effects the growth and development of transgenic plants (Esen et al., EMBO J. 10:2889-2895 (1991)).

Conjugation is often reported as a way of removing free and active hormones from a tissue. The conjugation process is often reversible, and, as conjugates can frequently accumulate in excess of free forms of phytohormone. The conjugate pools are also considered as sources of free hormone and may represent storage or inactive transportable forms of the hormone.

2. Gibberellin Metabolism

Gibberellins (“GAs”) are tetracyclic diterpenoid compounds found in fungi and higher plants. GAs are reported to regulate plant growth and development (Crozier, ed. Biochemistry and Physiology of Gibberellins, Vol. 2, Praeger, N.Y. (1983)). More than eighty different forms of naturally occurring, biologically active or inactive gibberellins have been identified (Sponsel, Plant Hormones, Physiology, Biochemistry and Molecular Biology ed. Davies, Kluwer Academic Publishers, Dordrecht, (1995)). These can be broadly categorized into C₂₀-GAs and C₁₉-GAs. A subset of active and inactive GAs may be found in any given plant species.

The GA biosynthetic pathway includes the following enzymes: copalyl diphosphate synthase, ent-kaurene synthase, ent-kaurene oxidase, cytochrome P450 monooxygenase, 7-oxidase, gibberellin 20-oxidase, 2β-hydroxylase, 3β-hydroxylase, gibberellin 2β, 3β-hydroxylase and GA enzymes capable of inactivation, 2β-hydroxylase, 3β-hydroxylase, gibberellin 2β, 3β-hydroxylase.

The first reported committed step in diterpenoid biosynthesis leading to gibberellins occurs when geranylgeranyl diphosphate is cyclized by copalyl diphosphate synthase (“CPS”, also referred to as ent-kaurene synthetase A) to copalyl diphosphate. GA biosynthesis is not eliminated in two reported mutants of the a encoding CPS, a gal mutant (Arabidopsis) and a an1 mutant (maize).

The second reported committed step in diterpenoid biosynthesis leading to gibberellins is a cyclization catalyzed by ent-kaurene synthase (“KS”, also referred to as ent-kaurene synthetase B), which converts copalyl diphosphate to ent-kaurene. KS exhibits amino acid homology to CPS and other terpene cyclases. Both CPS and KS are reported to be localized in developing plastids, which are generally found in vegetative tissues and seeds (Aach et al., Planta 197:333-342 (1995)).

Cytochrome P450 monooxegenases catalyze the oxidation of ent-kaurene. The products of this reaction are ent-kaurenol, ent-kaurenal, and/or ent-kaurenoic acid (Hedden and Kamiya, Ann. Rev. Plant Physiol. Plant Mol. Biol. 48:431-460 (1997)). An isolated maize cytochrome P-450 monooxegenase gene has been reported (Winkler and Helentjaris, Plant Cell 7:1307-1317 (1995)). Hydroxylation of ent-kaurenoic acid at position seven generates ent-7α-hydroxy-kaurenoic acid. From this intermediate, a contraction of the B ring generates GA₁₂-aldehyde.

An oxidation by 7-oxidase at C-7 of GA₁₂-aldehyde converts GA₁₂-aldehyde to GA₁₂-carboxylic acid. Beyond the formation of GA₁₂, the GA biosynthetic pathway is reported to vary in a species dependent manner. This oxidation is common to all GAs and is associated with biological activity. Both monooxygenases and 2-oxoglutarate dependent dioxygenases have been reported that can catalyze oxidation (Lange and Graebe, Methods in Plant Biochemistry, ed. Lea, Academic Press, London 9:403-430 (1993)).

One of the subsequent modifications is hydroxylation of the C-13 position resulting, for example, in the formation of GA₁. 13-hydroxylation may occur early in the gibberellin pathway (acting on the C-20 GA, GA₁₂ substrate) or late in the pathway during the interconversion of bioactive, non-13-hydroxylated, C-19 GAs to their 13-hydroxylated derivatives (e.g., GA₄ to GA₁). Generally, the formation of bioactive GAs includes successive oxidation of C-20 by Gibberellin 20-oxidase and the eventual loss of this carbon to create the C₁₉-GAs. Bioactive GAs undergo this oxidation and elimination step. This step in GA biosynthesis is a reported regulatory point that is responsive to environmental and feedback regulation (Xu et al., Proc. Natl. Acad. Sci. (U.S.A.) 92:6640-6644 (1995)). Enzyme substrate specificity can vary depending upon the species of origin. For example, rice GA 20-oxidase exhibits a reported substrate preference for 13-hydroxylated GAs, for example GA₅₃, and not for its non-13-hydroxylated precursor, GA₁₂ (Toyomasu et al., Plant Physiol. 99:111-118 (1997)).

GA 20-oxidase is a 2-oxoglutarate dependent dioxygenase that catalyzes the oxidation of C-20 GA₁₂ at position C-20. Genes encoding GA 20-oxidase have been isolated from several species including pumpkin, Arabidopsis and rice. Different members of GA 20-oxidase multigene family have been reported to be developmentally and spatially regulated (Phillips et al., Plant Physiol. 108:1049-1059 (1995)).

The final reported conversion necessary for the formation of bioactive GAs is the 30-hydroxylation catalyzed by 2-oxoglutarate dependent dioxygenase. Certain 3β-hydroxylases can hydroxylate more than one GA species. 3β-hydroxylase enzymes can also exhibit multifunctional capabilities and catalyze additional reactions such as a 2,3-desaturation and a 2β-hydroxylation (Smith et al., Plant Physiol. 94:1390-1401(1990), Lange et al., Plant Cell 9:1459-1467 (1997)).

Gibberellins can be rendered biologically inactive by several mechanisms. 2β-hydroxylation has been reported to eliminate GA activity. 2β-hydroxylation has also been reported as a GA inactivation mechanism in plants. Multiple enzymes with this activity may be present in a species (Smith and MacMillan, Journal of Plant Growth Regulators 2:251-264 (1984)). Bifunctional 2β, 3β-hydroxylase gene has been isolated from pumpkin endosperm (Lange et al., Plant Cell 9:1459-1467 (1997).

Further catabolism of 2β-hydroxylated GAs occurs by additional oxidation steps that can be catalyzed by 2-oxoglutarate dependent dioxygenases. GAs may also be inactivated or sequestered, in planta, by conjugation to sugars to form gibberellin glucosides and glucosyl ethers (Schneider and Schmidt, Plant Growth Substances, ed. Pharis, et al., Springer-Verlag, Heidelberg, 300 (1988)).

3. Ethylene Pathway

Ethylene is a plant hormone involved in the regulation of physiological responses (Abeles et al., Ethylene in Plant Biology, Second Edition New York: Academic Press, Inc. (1992); Mattoo et al., The Plant Hormone Ethylene, CRC Press, Inc. (1991)). In addition to its recognition as a “ripening hormone”, ethylene is reported to be involved in other developmental processes from germination of seeds to senescence of various organs (Davies, Plant Hormones: Physiology, Biochemistry and Molecular Biology, Dordrecht, Kluwer (1995)). Depending upon the type of plant, type of tissue, and/or developmental timing, ethylene regulates a variety of processes including fruit ripening, cell elongation, flower senescence, leaf abscission, and sex determination. Ethylene has also been implicated in the modulation of responses of plants to a wide range of biotic and abiotic stresses (Abeles et al., Ethylene in Plant Biology, Second Edition, New York: Academic Press, Inc. (1992); Yang and Hoffmann, Annu. Rev. Plant Physiol. 35:155-189 (1984)).

Ethylene is biosynthesized by some bacteria and fungi, but the biosynthetic pathway in microorganisms is different from the biosynthetic pathway in higher plants (Mattoo and Suttle, The Plant Hormone Ethylene, CRC Press, Inc (1991)). Ethylene is produced in most higher plants and is synthesized from methionine in tissues undergoing senescence or ripening.

As a gas, ethylene moves by diffusion from its site of synthesis. One intermediate in ethylene production, 1-aminocyclopropane-1-carboxylic acid (“ACC”) can be transported and may account for ethylene effects at a distance from the causal stimulus (Davies, Plant Hormones: Physiology, Biochemistry and Molecular Biology, Dordrecht: Kluwer (1995)).

Ethylene production is reported to be influenced by ethylene and other plant hormones. Auxin, cytokinins, abscisic acid and ethylene can regulate ethylene production at the level of ACC synthesis, although they may exert their effects by different biochemical mechanisms (Davies, Plant Hormones: Physiology, Biochemistry and Molecular Biology, Dordrecht: Kluwer (1995)).

The effects of ethylene on plant growth and development include the following: release from dormancy; shoot and root growth and differentiation (triple response); adventitious root formation; leaf, flower and fruit abscission; flower induction; induction of femaleness in dioecious flowers; flower opening; flower and leaf senescence; and fruit ripening (Davies, Plant Hormones: Physiology, Biochemistry and Molecular Biology, Dordrecht: Kluwer (1995)).

Several reviews have been published on the ethylene biosynthetic pathway that include: Abeles et al., Ethylene in Plant Biology, Second Edition, New York: Academic Press, Inc. (1992); Mattoo and Suttle, The Plant Hormone Ethylene, CRC Press, Inc (1991); Zarembinski and Theologis, Plant Mol. Biol. 26:1579-1597 (1994); and Kende, Annu. Rev. Plant Physiol. Plant Mol. Biol. 44:283-307 (1993), Fluhr and Mattoo, Crit. Rev. Plant Sci. 15:479-523 (1996).

Methionine is a biological precursor of ethylene. The ethylene pathway starts with the diversion of methionine into the SAM cycle which is also known as the methionine cycle. The SAM cycle generates S-adenosylmethionine (“AdoMet” or “SAM”) and other intermediates.

The enzyme S-adenosylmethionine synthetase (“SAM synthetase”) catalyzes the conversion of methionine and ATP into S-adenosylmethionine (AdoMet or SAM). This is the first reported step in the ethylene pathway.

In addition to its role in ethylene production, the precursor, S-adenosylmethionine (AdoMet or SAM) is involved in the biosynthesis of polyamines and in methylation reactions (Tabor and Tabor, Adv. Enzymology 56:251-282 (1984)).

SAM can be converted to decarboxylated S-adenosylmethionine (“dSAM”) by the enzyme S-adenosylmethionine decarboxylase (“SAM decarboxylase”) for polyamine biosynthesis. The enzyme S-adenosylmethionine hydrolase (“SAMase”) catalyzes the conversion of SAM to 5′-methylthioadenosine (MTA) and homoserine. SAM is the metabolic precursor of 1-aminocyclopropane-1-carboxylic acid (ACC), which itself is reported to be the immediate precursor of ethylene (Yang and Hoffmann, Annu. Rev. Plant Physiol. 35:155-189 (1984)).

The pathway from SAM to ethylene is catalyzed in higher plants by the enzymes ACC synthase (1-aminocyclopropane-1-carboxylic acid synthase) and ACC oxidase (1-aminocyclopropane-1-carboxylic acid oxidase) (also known as the ethylene-forming enzyme or EFE). The reported rate-limiting step in ethylene biosynthesis is the conversion of S-adenosylmethionine to ACC and 5′-methylthioadenosine (“MTA”) which is catalyzed by the enzyme ACC synthase.

The operation of a methionine cycle in plants results in recycling of MTA. MTA is produced as a product of ACC synthase and SAM decarboxylase, to regenerate methionine, and thereby SAM, providing a pathway to maximize the availability of SAM. The recycling of the methylthio group from SAM is important to the maintenance of ethylene production in plants.

ACC is then converted to either ethylene, CO₂, and HCN by the enzyme ACC oxidase or ACC is conjugated. Conjugation of the substrate ACC with malonate forms 1-(malonylamino)cyclopropane-1-carboxylic acid (M-ACC) by the enzyme ACC N-malonyltransferase (1-aminocyclopropane-1-carboxylate N-malonyltransferase). Another conjugate of ACC is 1-(L-glutamylamino) cyclopropane-1-carboxylic acid (G-ACC). Conjugation of ACC may be one way of sequestering the precursor to prevent its accumulation and conversion to ethylene.

The enzyme S-adenosylmethionine synthetase (SAM synthetase (EC 2.5.1.6)) catalyzes the conversion of methionine and ATP into S-adenosylmethionine (AdoMet or SAM). The genes for SAM synthetase, which catalyzes the conversion of methionine to SAM, have been cloned from Arabidopsis thaliana (Peleman et al., Plant Cell 1:81-93 (1989), Peleman et al., Gene 84:359-369 (1989)), from carnation (Woodson and Larsen, Plant Physiol. 96:997-999 (1991)), and poplar (Van Doorsselaere et al., Plant Physiol. 102:1365-1366 (1993)).

SAM synthetase gene is reported to be differentially expressed. The highest reported levels of expression for this gene is in vascular tissues, which requires AdoMet for lignification. It has been reported that levels of AdoMet synthase activity are adequate to allow for the autocatalytic production of ethylene that occurs during flower senescence (Woodson et al., Plant Physiol. 95:251-257 (1992)).

SAM can also be converted to S-decarboxylated S-adenosylmethionine (“dSAM”) by the enzyme S-adenosylmethionine decarboxylase (SAM decarboxylase). The propylamino group from SAM is added to putrescine to form polyamine spermine in the polyamine biosynthesis pathway (Abeles et al., Ethylene in Plant Biology, Second Edition, New York: Academic Press, Inc. (1992)).

The gene encoding the enzyme S-adenosylmethionine hydrolase (SAMase (EC 3.3.1.2)) has been isolated from the bacteriophage T3. SAMase catalyzes the conversion of SAM to 5′-methylthioadenosine (MTA) and homoserine. Expression of SAMase in transgenic plants has been reported to delay ripening of tomato fruit in a stage- and tissue-specific manner (Good et al., Plant Mol. Biol. 26:781-790 (1994)).

1-Aminocyclopropane-1-carboxylate synthase (ACC synthase (EC 4.4.1.14)), catalyzes the conversion of S-adenosylmethionine (AdoMet or SAM) to 1-aminocyclopropane-1-carboxylic acid (ACC) and 5′-methylthioadenosine (MTA), is reported to play a role in the regulation of ethylene production. The rate-limiting step in the synthesis of ethylene is reported to be the formation of ACC. ACC synthase exists in several isoforms which are derived from a divergent multigene family where each gene can be differentially regulated in response to developmental, environmental, and hormonal factors (Kende, Annu. Rev. Plant Physiol. Plant Mol. Biol. 44:283-307 (1993); Yang and Hoffmann, Annu. Rev. Plant Physiol. 35:155-189 (1984)). ACC synthase is a pyridoxal-5′-phosphate requiring enzyme and is reported to be sensitive to pyridoxal inhibitors, especially aminoethoxyvinylglycine (“AVG”) and aminooxyacetic acid (“AOA”) (Davies, Plant Hormones: Physiology, Biochemistry and Molecular Biology, Dordrecht: Kluwer (1995)).

An ACC synthase clone has been reported from zucchini (Sato and Theologis, Proc. Natl. Acad. Sci. (U.S.A.) 86:6621-6625 (1989)). Two ACC synthase clones have been reported from ripe tomato fruit (Van Der Straeten et al., Proc. Natl. Acad. Sci. (U.S.A.) 87:4859-4863 (1990)). ACC synthase clones have also been reported from different plant species, such as apple, tomato, Arabidopsis, winter squash, rice, orchid, carnation, mungbean hypocotyls, soybean, and tobacco (Zarembinski and Theologis, Plant Mol. Biol. 26:1579-1597 (1994); Fluhr and Mattoo, Crit. Rev. Plant Sci. 15:479-523 (1996)).

ACC synthase is reported to play a role in regulating ethylene biosynthesis. Increased ethylene production is reported to be involved in developmental processes including germination, ripening, and senescence, and in stress responses to wounding, drought, water logging, chilling, toxic agents, infection or insect infestation (Yang and Hoffmann, Annu. Rev. Plant Physiol. 35:155-189 (1984); Lieberman, Annu. Rev. Plant Physiol. 30:533-591 (1979)). It has been reported that the higher levels of ethylene are accompanied by increased ACC production, due to induction, based on increased transcription of ACC synthase gene(s) (Lincoln et al., J. Biol. Chem. 268:19422-19430 (1993); Dong et al., Planta 185:3845 (1991); Olson et al., Proc. Natl. Acad. Sci. (U.S.A.) 88:5340-5344 (1991); Olson et al., J. Biol. Chem. 270:14056-14061 (1995); Rottmann et al., J. Mol. Biol. 222:937-961 (1991); Clark et al., Plant Mol. Biol. 34:855-865 (1997); Huang et al., Proc. Natl. Acad. Sci. (U.S.A.) 88:7021-7025 (1991)). It has also been reported that the ACC synthase gene can be used to manipulate ethylene synthesis in plants, both positively (Lanahan et al., Plant Cell 6:521-530 (1994)) and negatively (Oeller et al., Science 254:437-439 (1991)).

Conjugation of 1-aminocyclopropane-1-carboxylic acid (ACC) to form 1-(malonylamino)cyclopropane-1-carboxylic acid (M-ACC) is catalyzed by the enzyme ACC N-malonyltransferase (1-aminocyclopropane-1-carboxylate N-malonyltransferase). This reaction constitutes a reported regulatory step by inactivating ACC. A rapid decline in the rate of ethylene production can result from decreased ACC synthesis, or from the conjugation of ACC to MACC. Ethylene production can be promoted by blocking malonylation (Yang and Hoffmann, Annu. Rev. Plant Physiol. 35:155-189 (1984); Su et al., Phytochemistry 24:1141-1145 (1985)). ACC N-malonyltransferase has been isolated and partially purified from mungbean hypocotyls (Guo et al., Plant Physiol. 100:2041-2045 (1992)) and from tomato fruit (Martin and Saftner, Plant Physiol. 108:1241-1249 (1995)).

An enzyme that synthesizes a conjugate of ACC, 1-(?-L-glutamylamino) cyclopropane-1-carboxylic acid (G-ACC), has been reported in tomato fruit (Martin et al., Plant Physiol. 109:917-926 (1995)). The enzyme is reported to use reduced glutathione as a substrate for this reaction. MACC is reported to be the major conjugate of ACC in plant tissues, whereas GACC is a minor conjugate (Peiser and Yang, Plant Physiol. 116:1527-1532 (1998)).

The enzyme 1-aminocyclopropane-1-carboxylate deaminase (ACC deaminase (EC 4.1.99.4)) degrades 1-aminocyclopropane-1-carboxylic acid (ACC) to alpha-ketobutyric acid and ammonia, thus effectively preventing its conversion to ethylene (Honma and Shimomura, Agric. Biol. Chem. 42:1825-1831 (1978)). The gene encoding ACC deaminase has been cloned from Pseudomonas sp. (Klee et al., Plant Cell 3:1187-1193 (1991); Sheehy et al., J. Bacteriol. 173:5260-5265 (1991)). Expression of this gene in plants is reported to reduce ethylene synthesis in all tissues where the gene is expressed.

The conversion of ACC to ethylene is reported to be carried out by an oxidative enzyme that is known as 1-aminocyclopropane-1-carboxylate oxidase (ACC oxidase) (formerly known as ethylene-forming enzyme or EFE) (Yang and Hoffmann, Annu. Rev. Plant Physiol. 35:155-189 (1984)). The conversion of ACC to ethylene is the reported final step in ethylene biosynthesis. ACC oxidase was identified by expressing the tomato cDNA pTOM13 in an antisense orientation, which reduced ethylene production in tomato fruit (Hamilton et al., Nature 346:284-287 (1990)). Ethylene is reported to be synthesized by an iron-dependent oxidation mechanism (Ververidis and John, Phytochemistry 30:725-727 (1991)). ACC oxidase activity was reported to be conferred when expressed in yeast (Hamilton et al., Proc. Natl. Acad. Sci. (U.S.A.) 88:7434-7437 (1991)) or Xenopus oocytes (Spanu et al., EMBO J. 10:2007-2013 (1991)).

ACC oxidase is encoded by multigene family. ACC oxidase has been purified from apple (Dong et al., Proc. Natl. Acad. Sci. (U.S.A.) 89:9789-9793 (1992); Dupille et al., Planta 190:65-70 (1993)). cDNAs for ACC oxidase have been isolated from different species, such as carnation, peach, melon, orchid, mungbean, broccoli, petunia, and tomato (Barry et al., Plant J. 9:525-535 (1996)). ACC oxidase has also been cloned from banana, melon and kiwi fruit (Huang et al., Biochem. Mol. Biol. Int. 41:941-950 (1997); Lasserre et al., Mol. Gen. Genet. 24:81-90 (1996); Lay et al., Eur. J. Biochem. 242:228-234 (1996)). ACC oxidase, as measured by ethylene production in the presence of a saturating concentration of ACC, is reported to be present in most tissues of higher plants. However, under some stress conditions, in response to ethylene, or during certain developmental stages (such as fruit ripening), the level of ACC oxidase increases and is reported to effectively regulate ethylene production (Yang and Hoffmann, Annu. Rev. Plant Physiol. 35:155-189 (1984)). Antisense gene expression of ACC oxidase has been reported to reduce ethylene synthesis in ripening fruit by 97% relative to the controls (Hamilton et al., Nature 346:284-287 (1990)). ACC oxidase antisense gene has also been reported to delay leaf senescence (Picton et al., Plant Journal 3:469-481 (1993)).

Ethylene synthesis is reported to be controlled at the level of ACC synthase. It has also been reported that ACC oxidase also plays a role in regulating ethylene biosynthesis (Kende and Zeevaart, Plant Cell 9:1197-1210 (1997)).

Ethylene is reported to activate transcription of a set of genes in ripening tomato fruit, including E4 and E8 (Lincoln et al., Proc. Natl. Acad. Sci. (U.S.A.) 84:22793-2797 (1987)). Regulation of the E4 and E8 genes by ethylene is reviewed by Deikman, Physiol. Planta. 100:561-566 (1997). The E8 gene has been reported to be transcriptionally activated at the onset of ripening (Lincoln et al., Proc. Natl. Acad. Sci. (U.S.A.) 84:22793-2797 (1987)), and the E8 protein has been reported, by sequence homology, to be a member of the dioxygenase family of enzymes which includes the ACC oxidase family identified by Hamilton et al., Nature 346:284-287 (1990); Hamilton et al., Proc. Natl. Acad. Sci. (U.S.A.) 88:7434-7437 (1991); Deikman et al., EMBO J. 7:3315-3320 (1988). E8 gene expression leads to the inhibition of ethylene production in tomato (Lincoln and Fischer, Plant Physiol. 88:370-374 (1988)). Transgenic tomato fruit expressing antisense E8 mRNA have shown that this gene negatively regulates ethylene biosynthesis in fruit. (Penarrubia et al., Plant Cell 4:681-687 (1992)). It has been reported that the E8 protein may constitute a part of the proposed metalloprotein ethylene receptor. A cDNA clone 2A6, from Arabidopsis shows high homology to the tomato E8 cDNA. The 2A6 protein shows three domains that are highly conserved among E8, ACC oxidases, and 2-oxoglutarate-dependent dioxygenases (2-ODD) (Trentmann et al., Plant Mol. Biol. 29:161-166 (1995).

Ethylene synthesis has been reduced using antisense gene constructs of ACC oxidase (Hamilton et al., Nature 346:284-287 (1990)) or ACC synthase (Oeller et al., Science 254:437-439 (1991)) and by expressing ACC deaminase (Klee et al., Plant Cell 3:1187-1193 (1991)).

The ethylene transduction pathway has been studied by screening for ethylene response mutants in Arabidopsis (Ecker, Science 268:667-675 (1995)). Mechanisms responsible for the perception of the ethylene stimulus or how signals are transduced following perception in order to bring about specific alterations to gene expression are reviewed by Bleecker and Schaller, Plant Physiol. 111:653-660 (1996); Chang, Trends Biochem. Sci. 21:129-133 (1996); Theologis, Curr. Biol. 6:144-145 (1996); Theologis, Science 270:1774 (1995); Ecker, Science 268:667-675 (1995); Kieber and Ecker, Trends Genet. 9:356-362 (1993); Bleecker, Symp. Soc. Exp. Biol. 45:149-158 (1991).

A seedling triple response phenotype in Arabidopsis has been used to dissect the components of the ethylene response pathway genetically, and several classes of ethylene-related mutants have been identified (Ecker, Science 268:667-675 (1995)). In the presence of ethylene, dark-grown seedlings of such mutants either do not exhibit the “triple response” or show the triple response phenotype even in the absence of ethylene. Chemical inhibitors of ethylene biosynthesis or binding, or mutations that block the perception of ethylene are reported to prevent this morphological transformation (Guzman and Ecker, Plant Cell 2:513-523 (1990)).

One group of mutants includes those that display ethylene responses in the absence of exogenous ethylene. Plants that display a constitutive triple response phenotype (Ctr) may result either from ethylene overproduction, as is the case for eto1, eto2, and eto3 mutants (Guzman and Ecker, Plant Cell 2:513-523 (1990); Kieber et al., Cell 72:427-441 (1993)), or as a consequence of constitutive activation of the ethylene signaling pathway, as is the case for the ctr1 (CONSTITUTIVE TRIPLE RESPONSE1) mutant (Kieber et al., Cell 72:427-441 (1993)).

A second class of mutants includes those that show a reduction or absence of responsiveness to treatment with exogenous ethylene. Insensitive or resistant mutants are reported to be altered in their ability to perceive or respond to ethylene, and include etr1 (Bleecker et al., Science 241:1086-1089 (1988)); ein2 (Guzman and Ecker, Plant Cell 2, 513-523 (1990)); ein3 (Rothenberg and Ecker, Sem. Dev. Biol. Plant Dev. Genet. 4:3-13 (1993); Kieber and Ecker, Trends Genet. 9:356-362 (1993)); ain1 (Van Der Straeten et al., Plant Physiol. 102:401-408 (1993)); eti mutants (Harpham et al., Ann. Bot. 68:55-62 (1991)); and ein4, ein5, ein6, and ein7 (Roman et al., Genetics 139:1393-1409 (1995)).

The mutation ethylene-resistant1 (etr1) is dominant, and the mutant lacks a number of responses to ethylene (Bleecker et al., Science 241:1086-1089 (1988)). The capacity of etr1 to bind ethylene in vivo was reported to be one-fifth that of the wild-type, indicating that the mutant is impaired in receptor function. An ETR1 gene has been cloned and found to encode a protein with sequence similarity to bacterial two-component regulators (Chang et al., Science 262:539-544 (1993); Chang and Meyerowitz Proc. Natl. Acad. Sci. (U.S.A.) 92:4129-4133 (1995); Chang et al., Biochem. Soc. Trans. 20:73-75 (1992)). ETR1 is reported to form membrane-associated dimers and, when expressed in yeast binds ethylene (Schaller and Bleecker, Science 270:1809-1811 (1995); Schaller et al., J. Biol. Chem. 270:12526-12530 (1995)).

The ETR2 and ETHYLENE-INSENSITIVE4 (EIN4) genes encode homologues of ETR1, and mutations in these genes confer dominant ethylene insensitivity onto Arabidopsis seedlings (Roman et al., Genetics 139:1393-1409 (1995); Hua et al., Science 269:1712-1714 (1995)). An ETHYLENE RESPONSE SENSOR (ERS) gene of Arabidopsis encodes a second type of putative ethylene receptor which was reported to confer dominant ethylene insensitivity. ERS acts upstream of the CTR1 protein kinase gene in the ethylene response pathway (Hua et al., Science 269:1712-1714 (1995)). Homologues of ETR1 and ERS1 have also been isolated from tomato and include Never ripe (Wilkinson et al., Science 270:1807-1809 (1995)), tETR1 (tomato ETR1) (Payton et al., Plant Mol. Biol. 31:1227-1231 (1996)), eTAE (Zhou et al., Plant Mol. Biol. 30:1331-1338 (1996)) and a third ETR1-related gene, TFE27 (Zhou et al., Plant Physiol. 110:1435-1436 (1996)).

The tomato Never ripe locus is reported to regulate ethylene-inducible gene expression and has been reported to be linked to a homologue of the Arabidopsis ETR1 gene (Yen et al., Plant Physiol. 107:1343-1353 (1995)). Never ripe mutants are reported to contain reduced amounts of polygalacturonase (Tucker et al., Eur. J. Biochem. 112:119-124 (1980)). It has also been reported that the Never ripe mutation blocks ethylene perception in tomatoes (Lanahan et al., Plant Cell 6:521-530 (1994)).

It has been reported that tETR mRNA is undetectable in unripe fruit or pre-senescent flowers, and increases in abundance during the early stages of ripening, flower senescence and in abscission zones, and is reduced in fruit of ripening mutants deficient in ethylene synthesis or response (Payton et al., Plant Mol. Biol. 31:1227-1231 (1996)).

eTAE mRNA has been reported to be constitutively expressed in all tissues, and its accumulation in leaf abscission zones is reported to be unaffected by ethylene, silver ions (an inhibitor of ethylene action) or auxin (Zhou et al., Plant Mol. Biol. 30:1331-1338 (1996)).

An EIN2 gene has been cloned. A sequence of the E1N2 gene is reported in PCT publication number WO 95/35318.

EIN3 encodes a novel nuclear-localized protein that shares sequence similarity, structural features, and genetic function with three EIN3-LIKE (EIL) proteins. EIN3 (ETHYLENE-INSENSITVE3) encodes a positive regulator in the ethylene signaling pathway of Arabidopsis. Several related EIN-LIKE (EIL1, EIL2, EIL3) genes have also been cloned, and like EIN3, which was localized to the nucleus, their predicted translation products contain features commonly found in transcriptional regulatory proteins. It has been reported that EIN3 is a downstream regulator in the ethylene signaling pathway and may act along with EIL proteins to mediate a diverse array of plant responses to ethylene gas (Chao et al., Cell 89:1133-1144 (1997)). Mutations in the Arabidopsis ETHYLENE-INSENSITIVE3 (EIN3) gene are reported to limit a plant's response to the gaseous hormone ethylene (Chao et al., Cell 89:1133-1144 (1997)).

Genes acting downstream of ethylene perception in Arabidopsis include CONSTITUTIVE TRIPLE RESPONSE1 (CTR1). A gene corresponding to the ctr1 mutation has been cloned and reported to encode a peptide that resembles the Raf family of serine/threonine kinases. CTR1 protein is reported to act as a negative regulator in the ethylene signal transduction pathway. Ctrl mutants express the triple response phenotype constitutively, even in the absence of ethylene (Kieber et al., Cell 72:427-441 (1993)). It has been reported that ctr1 mutants affect the production of root hair and hairless cells in the Arabidopsis root (Masucci and Schiefelbein, Plant Cell 8:1505-1517 (1996)).

A genetic framework has been reported for the action of these genes in the ethylene response pathway (Roman et al., Genetics 139:1393-1409 (1995)). These reports set forth that ETR1 and EIN4 act upstream of the CTR1, whereas the EIN2, EIN3, EIN5, EIN6, and EIN7 act downstream of CTR1.

The downstream branches identified by the EIR1, AUX1 and HLS1 genes may involve interactions with other hormonal or developmental signals (Roman et al., Genetics 139:1393-1409 (1995)).

The HOOKLESS1 (HLS1) gene of Arabidopsis has been identified as an ethylene-responsive gene whose expression is required for the formation of the apical hook (Lehman et al., Cell 85:183-194 (1996)). It has been reported that the N-acetyl transferase encoded by HLS1 affects the distribution of auxin in seedlings and constitute a link between ethylene and auxin action in asymmetric growth.

An ethylene-induced cDNA clone encoding a protein kinase, PK12, has been isolated (Sessa et al., Plant Cell 8:2223-2234 (1996)). The activation characteristics of PK12 kinase, suggests its involvement in the ethylene signal transduction pathway (Fluhr, Trends Plant Sci. 3:141-146 (1998)).

4. Jasmonic Acid Synthesis Pathway

Jasmonic acid is a cyclopentanone containing compound that accumulates in response to wounding or pathogen attack (Mueller, Physiologia Plantarum 100:653-663 (1997)). Jasmonic acid has been reported to modulate responses to environmental stimuli such as wounding or pathogenic attack (Wasternack and Parthier, Trends in Plant Sciences 2:302-307 (1997)), and developmental processes, such as germination, senescence, fruit development, production of viable pollen, and root growth (Creelman and Mullet, Annual Review of Plant Physiology and Plant Molecular Biology 48:335-381 (1997)). Jasmonic acid is derived from α-linolenic acid via four enzymes that are specific to jasmonic acid synthesis. These enzymes are a lipoxygenase (E.C. 1.13.11.12), allene oxide synthase (E.C. 4.2.1.92), allene oxide cyclase (E.C. 5.3.99.6), and 12-oxo-phytodienoic acid reductase. The product of 12-oxo-phytodienoic acid reductase, 10,11 dihydro-12-oxo-phytodienoic acid, is converted to jasmonic acid by three rounds of β-oxidation (Mueller, Physiologia Plantarum 100:653-663 (1997)).

Lipoxygenase (LOX) is a non-heme iron containing enzyme that catalyzes the formation of hydroperoxylinolenate from linolenic acid. Multiple forms of LOX are found in plants that vary in their organ and tissue distribution. LOX isoforms also differ in their substrate specificity and production of hydroperoxylinolenate isomers. LOX is generally located in multiple subcellular compartments. In plants, LOX produces two linolenic acid derived isomers, 9-hydroperoxylinolenate and 13-hydroperoxylinolenate. 13-hydroperoxylinolenate is reported to be a precursor to jasmonic acid. LOX activity that is associated with the formation of 13-hydroperoxylinolenate during inducible jasmonic acid production has been reported in the plastid (Creelman and Mullet, Annual Review of Plant Physiology and Plant Molecular Biology 48:335-381 (1997)). A rice LOX, containing a plastid transit sequence, is reported to catalyze the production of 13-hydroperoxylinolenate (Peng et al., J. Biol. Chem. 269:3755-3761 (1994)). Transgenic Arabidopsis with a reduced accumulation of plastidic LOX2 failed to generate a jasmonic acid response to wounding (Bell et al., Proc. Natl. Acad. Sci. (U.S.A.) 92:8675-8679 (1995)). Non-plastidic LOX has been reported to be associated with the constitutive production of jasmonic acid.

Allene oxide synthase (AOS (EC 4.2.1.92)) catalyzes the conversion of 13-hydroperoxylinolenate to 12,13-epoxy-octadecatrienoic acid. Allene oxide synthase has been cloned from flax (Song et al., Proc. Natl. Acad. of Sci. (U.S.A.) 90:8519-8523 (1993)), guayule (Pan et al., J. Biol. Chem. 270:8487-8494 (1995)), and Arabidopsis (Laudert et al., Plant Molecular Biology 31:323-335 (1996)). AOS is a cytochrome P450 enzyme with a heme binding region in the C-terminal portion of the protein. Reported flax and Arabidopsis clones encode proteins with plastid transit sequences (Song et al., Proc. Natl. Acad. Sci. (U.S.A.) 90:8519-8523 (1993); Laudert et al., Plant Molecular Biology 31:323-335 (1996)). A reported flax clone has been expressed in potato and the AOS protein accumulated in plastids. Transgenic potato plants expressing flax AOS are reported to have elevated levels of jasmonic acid. These reported elevated jasmonic acid levels did not result in constitutive expression of wound induced transcripts (Harms et al., Plant Cell 7:1645-1654 (1995)). A cloned Arabidopsis AOS has been expressed in E. coli and is reported to accept either 9-hydroperoxylinolenate or 13-hydroperxoylinolenate as substrates (Laudert et al., Plant Molecular Biology 31:323-335, (1996). A cloned guayule AOS is homologous to clones from Arabidopsis and flax. In guayuale, AOS expression is reported to be associated with rubber particles in the bark parenchyma (Pan et al., J. Biol. Chem. 270:8487-8494 (1995)).

Allene oxide cyclase (AOC (EC 5.3.99.6)) catalyzes the conversion of 12,13-epoxy-octadecatrienoic acid to 12-oxo-phytodienoic acid. AOC activity has been reported in the seed coat of immature soybean seeds (Simpson and Gardner, Plant Physiology 108:199-202 (1995)) and dry maize seeds (Ziegler et al., Plant Physiology 114:565-573 (1997)). A maize AOC enzyme was reported to be soluble, with an approximately molecular weight of 45-47 kD. The reported substrate specificity of the maize AOC is 12,13-epoxy-octadecatrienoic acid, which is derived from linolenic acid. AOC exhibits only limited activity with the substrate 12,13-epoxy-octadecadienoic acid, which is derived from linoleic acid (Ziegler et al., Plant Physiology 114:565-573 (1997)).

12-oxo-phytodienoic acid reductase catalyzes the reduction of the 10,11 double bond of 12-oxo-phytodienoic acid to form 10,11-dihydro-12-oxo-phytodienoic acid. The enzyme has been characterized from maize seedlings and kernels (Vick and Zimmerman, Plant Physiology 80:202-205 (1986)), and suspension cultures of Corydalis sempervirens (Schaller and Weiler, Eur. J. Biochem. 245:294-299 (1997)). A maize 12-oxo-phytodienoic acid reductase enzyme was reported to be soluble. Based on gel filtration measurements, the estimated molecular weight of 12-oxo-phytodienoic acid reductase is 54 kD (Vick and Zimmerman, Plant Physiology 80:202-205 (1986)). A Corydalis 12-oxo-phytodienoic acid reductase enzyme has a reported monomer molecular weight of approximately 41 kD (Schaller and Weiler, Eur. J. Biochem. 245:294-299 (1997)). 12-oxo-phytodienoic acid reductase exhibits a co-factor preference for NADPH over NADH, and the maize enzyme is reported to be active over a broad pH range (Schaller and Weiler, Eur. J. Biochem. 245:294-299 (1997); Vick and Zimmerman, Plant Physiology 80:202-205 (1986)).

5. Transcription Factors

Eukaryotic transcription utilizes three different RNA polymerases. RNA polymerase I is located in the nucleolus and catalyzes the synthesis of ribosomal RNA. RNA polymerase II and III are present in the nucleoplasm. DNA dependent RNA synthesis by RNA polymerase III transcription complexes is responsible for the transcription of the genes that encode small nuclear RNAs and transfer RNA. RNA polymerase II transcribes the majority of the nuclear structural genes which typically encode proteins (type II genes).

In higher eukaryotes type II gene expression is often regulated, at least in part, at the level of transcription. A typical type II gene has one or more regulatory regions which include a promoter and one or more structural regions which is transcribed into precursor and messenger RNA. Type II genes are characterized by an upstream promoter region. Such regions are typically found between the start of transcription and 2000 bases distal to that transcriptional start site. Different combinations of sequence motifs can be associated with the upstream promoter region. These sequence motifs are recognized by sequence specific DNA binding proteins (transcription factors).

The polypeptide chains of transcription factors are usually divided into two functionally different regions, one that specifically binds to nucleic acid molecules and another that is associated with the activation of transcription. These functions are often present on different domains.

Several distinct structural elements or DNA binding domains which allow the transcription factor to bind to DNA in a sequence specific manner have been identified (Branden and Tooze, Introduction to Protein Structure, Garland Publishing, Inc., New York (1990)). These binding domains often range in size from approximately 20 residues to more than 80 residues. Many DNA binding domain exhibit one or another of the following structural motifs: the helix-turn-helix motif, the zinc finger motif, and the leucine zipper motif. Other structural motifs include: the helix-loop-helix motif, the pou motif and the multi-cysteine zinc finger.

Two sequence motifs or cis elements, the TATA box and the CAAT box are located within the promoter region of most type II genes. An AT-rich sequence called a TATA box is located approximately 30 nucleotides upstream from the start of transcription and is reported to play a role in positioning the start of transcription. A TATA box binding protein or TFIID factor has been identified that binds to this region (Hancock, Nucleic Acid Research 21:2823-2830 (1993); Gasch et al., Nature 346:390-394 (1990))(the TFIID factor is also referred to as the TBP/TAF factors). It has been reported that binding of TFIID to the TATA box plays a role in the assembly of other transcription factors to form a complex capable of initiating transcription (Nakajima et al., Mole. Cell. Biol. 8:4038-4040 (1988); Van Dyke et al., Science 241:1335-1338 (1988); Buratowski et al., Cell 56:549-561 (1989)).

In addition to the TATA box sequence, a CAAT box sequence is usually located approximately 75 bases upstream of the start of transcription. A CAAT box sequence binds a number of proteins, some of which are expressed in all tissues while others are expressed in a tissue specific manner (Branden and Tooze, Introduction to Protein Structure, Garland Publishing, Inc., New York (1990). One example of a CAAT box binding protein is the protein referred to as the CAAT box binding protein (C/EBP).

The G-box is a cis-acting element found within the promoters of many plant genes where it mediates expression in response to a variety of different stimuli (Schindler et al., EMBO J. 11:1275-1289 (1992)). The G-box comprises a palindromic DNA motif (CACGTG) which is composed of two identical half sites (Donald et al., EMBO J. 9:1727-1735 (1990); Izawa et al., J. Mol. Biol. 230:1131-1144 (1993) Schindler et al., Plant Cell 4:1309-1319 (1992); Schindler et al., EMBO J. 11:1275-1289 (1992); Odea et al., EMBO J. 10: 1793-1991 (1991) Weisshaar et al., EMBO J. 10: 1777-1786 (1991); and Zhang et al., Plant J. 4:711-716 (1993)). Both half sites are involved in the binding of the bZIP protein, GBF1, a member of the family Arabidopsis thaliana. The bZIP protein has been characterized in at least 19 other plant species (Erlich et al., Gene 117:169-178 (1992); Foley et al., Plant J. 3:669-679 (1993); Guiltinan et al., Science 250:267-271 (1990); Kawata et al., Nucl. Acids Res. 20:1141 (1992); Katagiri et al., Nature 340:727-730 (1989); Odea et al., EMBO J. 10:1793-1991 (1991); Pysh et al., Plant Cell 5:227-236 (1993); Schindler et al., Plant Cell 4:1309-1319 (1992); Schmidt et al., Proc. Natl. Acad. Sci. (USA) 87:46-50 (1990); Singh et al., Plant Cell 2: 891-903 (1990); Tabata et al., EMBO J. 10: 1459-1467 (1991); Tabata et al., Science 245:965-967 (1989); Weisshaar et al., EMBO J. 10:1777-1786 (1991); Zhang et al., Plant J. 4:711-716 (1993)). Each of these proteins recognizes DNA sequences that share the central core sequence ACGT. bZIP transcription factors are characterized by the presence of a basic domain and a leucine zipper.

Plant bZIP proteins have been shown to bind regulatory elements from a wide variety of inducible plant genes including those regulated by cell cycle, light, UV light, drought and pathogen infections (Ehrlich et al., Gene 117:169-178 (1992), Donald et al., EMBO J. 9:1727-1735 (1990); Guiltinan et al., Science 250:267-271 (1990); Katagiri et al., Nature 340:727-730 (1989); Oeda et al., EMBO J. 10:1793-1991 (1991); Tabata et al., EMBO J. 10:1459-1467 (1991); Weisshaar et al., EMBO J. 10:1777-1786 (1991); Holdworth et al., Plant Molecular Biology 29:711-720 (1995); Mikami et al., Mol. Gen. Genet. 248: 573-582 (1995)).

Specific transcription factors contribute to the quantitative and qualitative gene expression within a cell. The activity of a given transcription factors can effect cell physiology, metabolism, and/or the cell's ability to differentiate and communicate or associate with other cells within an organism. The regulation of the transcription of a gene may be the result of the activity of one or more transcription factors. Transcription factors are involved in the regulation of constitutive expression, inducible expression (such as expression in response to an environmental stimuli), and developmentally regulated expression.

Transcription factor gene families have been reported in plants (Martin and Paz-Ares, Trends in Genetics 13:43-84 (1997); Riechmann and Meyerowitz, Bio. Chem. 378:1079-1101 (1997)). The MADS-box transcription factor family is one example of a transcription factor gene family found in plants as well as other organisms (Riechmann and Meyerowitz, Bio. Chem. 378: 1079-1101 (1997); Noda et al., Nature 369:661-664 (1994); Schwarz-Sommer et al., EMBO J. 11:251-263 (1992); Yanofsky et al., Nature 346:35-39 (1990); Drews et al., Cell 65: 991-1002 (1991); Mizukami and Ma, Cell 71:119-131 (1992); Mandal et al., Nature 360:273-277 (1992); Gustafson-Brown et al., Cell 76:131-143 (1994); Jack et al., Cell 68:703-716 (1992); Goto and Meyerowitz, Genes and Development 8:1548-1560 (1994); Kriek and Meyerowitz, Development 122:11-22 (1996); Kempin et al., Science 267:522-525 (1995); Ma et al., Genes and Development 5:484-495 (1991); Flanagan et al., Plant J. 10:343-353 (1996); Flanagan and Ma, Plant Mol. Biol. 26:581-595 (1994); Huang et al., Plant Cell 8:81-94 (1995); Savidge et al., Plant Cell 7.721-733 (1995); Mandal and Yanofsky, Plant Cell 7:1763-1771 (1995); Roundsley et al., Plant Cell 7:1259-1269 (1995); Heck et al., Plant Cell 7:1271-1282 (1995); Perry et al., Plant Cell 8:1977-1989 (1996); Bradley et al., Cell 72:85-95 (1993); Huijser et al., EMBO J. 11:1239-1249 (1992); Sommer et al., EMBO J. 9:605-613 (1990); Trober et al., EMBO J. 11:4693-4704 (1992); Schwarz-Sommer et al., EMBO J. 11:251-263 (1992); Davies et al., EMBO J. 15:4330-4343 (1996); Zachgo et al., Development 121:2861-2875 (1995); Tsuchimoto et al., Plant Cell 5:843-853 (1993); Angenent et al., Plant J. 5:33-44 (1993); Van der Krol et al., Genes and Development 7:1214-1228 (1993); Angenent et al., Plant Cell 7:505-516 (1995); Angenent et al., Plant Cell 4:983-993 (1992); Angenent et al., Plant J. 5:33-44 (1994); Angenent et al., Plant J. 4:101-112 (1993); Angenent et al., Plant Cell 7:1569-1582 (1995); Columbo et al., Plant Cell 7:1859-1868 (1995)).

MADS-box transcription factors have been shown to bind to DNA and alter transcription by both induction and repression. Examples are known where MADS-box transcription factors exert their transcriptional regulation by binding and interacting individually, as homodimers or heterodimers, or through heterologous associations with non-MADS-box transcription factors. However, MADS transcription factors typically form dimers (Riechmann and Meyerowitz, Bio. Chem. 378:1079-1101 (1997)). MADS box transcription factors are defined by the signature MADS domain which is the most highly conserved portion of the protein among all the family members. In plants, additional domains (the I region, K-domain, and C-terminal region, in linear order) have been reported which are characteristic of the plant specific branch of this family.

The MADS domain is an approximately 57 amino acid domain located at or near the N-terminal portion of the MADS-box transcription factor (with approximately 260 amino acids in the total protein). This domain is highly conserved and is the most uniquely defining element of the family. For example, two homologues, APETALA1 from Arabidopsis and ZAP1 from Zea mays, show 89% identity over MADS domain. Conservation of this domain may be linked to its function as the portion of the protein that directly interacts with the target DNA binding site. The MADS domain is responsible for specifically binding DNA at A-T rich sequences referred to as CArG-boxes, whose consensus sequence has been reported as CC(A/T)₆GG (Shore and Sharrocks, Eur. J. Biochem. 229:1-13 (1995)).

The I domain spans approximately 30 amino acid sequence of poor sequence conservation compared to the MADS-domain. The intervening-region links the MADS domain region with the K-domain. Its length and sequence is variable and may be absent from some family members.

The K domain is an approximately 70 amino acid domain that is unique to the plant family members of the MADS-box gene superfamily. It is found in the majority of plant MADS-box genes. It has weak similarity to portions of animal keratin and is predicted to form amphipathic alpha helices which may facilitate interaction with other proteins. It has been reported that the structural conformation of this domain is a contributing constraint on conservation of this sequence. The K-domain typically exhibits less overall amino acid conservation than the MADS-domain, but between homologue genes such as APETALA1 from Arabidopsis and ZAP1 from maize, this similarity can still be high (approximately 70%).

The C terminal domain, along with the I-domain, is the least conserved portions of the MADS-box gene family member in plants. Although exact functions for this approximately 90-100 amino acid domain have not been determined, there are known mutations within this region that lead to distinct developmental abnormalities in plants which indicate a role in transcriptional regulation. Conservation of this domain increases with increasing evolutionary closeness of species and homologues under comparison.

Genetic and molecular analysis have shown that transcription factors belonging to the MADS transcription factor family, at least in part, regulate diverse functions (Riechmann and Meyerowitz, Bio. Chem. 378:1079-1101 (1997)). MADS transcription factors often exert their effect in a homeotic manner (e.g. loss of AG activity (a MADS transcription factor) in Arabidopsis homeotically transforms the third and fourth whorl organs and eliminates floral determinacy) (Mena et al., Science 274:1537-1540 (1996)). MADS transcription factors can regulate different processes. For example, the role of certain MADS transcription factors in floral development is reviewed in Riechmann and Meyerowitz, Bio. Chem. 378:1079-1101 (1997)). MADS transcription factors are also involved in the regulation of other plant processes such as phytochrome regulation (Wang et al., Plant Cell 9:491-507 (1997)) and seed development (Colombo et al., Plant Cell 9:703-715 (1997)).

Another family of transcription factors found in plants are MYB transcription factors. MYB transcription factors generally contain three repeats (R1, R2 and R3). The MYB DNA binding domain of plant proteins usually consists of two imperfect repeats of about 50 residues (Baranowskij et al., EMBO J. 13:5383-5392 (1994)). MYB transcription factors exhibit a helix-turn-helix motif (Ogata et al., Cell 79:639-648 (1994)). The DNA binding specificity of plant MYB proteins differs. For example, the maize P protein recognizes the motif [C/A]TCC[T/A]ACC similar to that bound by AmMYB305 from Antirhinum, and neither of these proteins appears to bind to the similar vertebrate MYB consensus motif (TAACNG) (Grotewold et al., Cell 76:543-553 (1994); Solano et al., EMBO J. 14:1773-1784 (1995)). Small changes in the amino acid sequence of a MYB transcription factor can alter the DNA binding properties of that transcription factor. For example, PMYB3 from Petunia binds to two sequences, MBSI (TAAC[C/G] GTT) and MBSII (TAACTAAG) (Solano et al., EMBO J. 14:1773-1784 (1995)). In the case of PMYB3, it has been shown that a substitution of a single residue in the R2 recognition helix switches the dual DNA-binding specificity to that of c-MYB, and the reciprocal substitution in c-MYB gives dual DNA-binding specificity similar to PhMYB3.

Mutations in residues that do not contact bases may also effect sequence-specific binding and have been reported to account for some of the differences in DNA-binding specificity between plant MYB proteins (Suzuki, Proc Jap. Acad. Series B 71:27-31 (1995)). Of the eight putative base-contacting residues in MYB proteins, six are fully conserved in all plant MYB proteins, and the remaining two are conserved in at least 80% of these proteins. Nonetheless MYB transcription factors exhibit different nucleic acid sequence specificities and different strengths of contacts (Solano et al., Plant J. 8:673-682 (1995)). In addition, temporal patterns of accumulation of RNA of different plant MYB genes may be effected by environmental stimuli, such as light, salt stress or the plant hormones, gibberellic acid and abscisic acid (Urao et al., Plant Cell 5:1529-1539 (1993); Jackson et al., Plant Cell 3:115-125 (1991); Cone et al., Plant Cell 5:1795-1805 (1993); Noda et al., Nature 369:661-664 (1994); Larkin et al., Plant Cell 5:1739-1748 (1993); Gubler et al., Plant Cell 7:1879-1891 (1995); Hattari et al., Genes Dev. 6:609-618 (1992)).

In plants distinct functions for different MYB transcription factors have been reported including controlling secondary metabolism, regulation of cellular morphogenesis and the signal transduction pathways. MYB proteins are reported to play a role in the control of phenylpropanoid metabolism. Phenylpropanoid metabolism is one of the three main types of secondary metabolism in plants involving modification of compounds derived initially from phenylalanine. Through one branch (flavonoid metabolism) it is responsible for the production of a majority group of plant pigments (the anthocyanins) and other minor groups (aurones and phlobaphenes) and it also produces compounds that modify pigmentation through chemical interaction with the anthocyanins (co-pigmentation), such as the flavones and flavonols. Flavones and flavonols also serve to absorb ultraviolet light to protect plants. Several flavanoids act as signalling molecules in legumes inducing gene expression in symbiotic bacteria in a species-specific manner, and others act as factors required for pollen maturation and pollen germination in some plant species. A number of flavanoids and related phenylpropanoids (such as stilbenes) also act as defensive agents (phytoallexins) against biotic and abiotic stresses in particular plant species. Another branch of phenylpropanoid metabolism produces the precursors for production of lignin, the strengthening and waterproofing material of plant vascular tissue and one of the principal components of wood. This branch also produces other soluble phenolics, which can serve as signalling molecules, cell-wall crosslinking agents and antioxidants.

The C1 transcription factor (a MYB transcription factor) activates transcription of genes encoding enzymes involved in the biosynthesis of the anthocyanin pigments in the outer layer of cells of the maize seed endosperm (the aleurone) (Paz-Ares et al., EMBO J. 5:829-833 (1986) Cone et al., Proc. Natl. Acad. Sci. (U.S.A.) 83:9631-9635 (1986)). Activation has been reported for at least five genes in the pathway to anthocyanin. Activation by C1 involves a partner transcriptional activator found in aleurone, a protein similar to a MYB transcription factor. These proteins also interact with other members of the R-protein family to regulate anthocyanin biosynthetic gene expression (Cone et al., Plant Cell 5:1795-1805 (1993)). For example, in maize, another MYB protein, ZmMYB1, can activate one of the structural genes required for anthocyanin production (Franken et al., Plant J. 6:21-30 (1994)), while yet another, ZmMYB38, inhibits C1-mediated activation of the same promoter.

Reiteration of MYB-gene function reportedly occurs in the control of a branch of flavonoid metabolism producing the red phlobaphene pigments from intermediates in flavonoid metabolism. This pathway is under control of the P gene in maize, which encodes a MYB-related protein (Grotewold et al., Cell 76:543-553 (1994)). The P gene product activates a subset of the genes involved in anthocyanin biosynthesis. The P-binding site is contained within the promoters of these target genes (Li and Parish, Plant J. 8:963-972 (1995)). In maize, at least two different MYB proteins serve to direct flavonoid metabolism along different routes by selective activation of target genes.

In other plant species MYB proteins can serve similar roles in the control of phenylpropanoid metabolism as, for example, in Petunia flowers. MYB proteins can also serve to regulate other branches of phenylpropanoid metabolism. In Antirrhinum majus and tobacco AmMYB305 (or its homologue in tobacco) can activate the gene encoding the first enzyme of phenylpropanoid metabolism, phenylalanine ammonia lyase (PAL (Urao et al., Plant Cell 5:1529-1539 (1993)). Some MYB genes have been shown to be highly expressed in tissues such as differentiating xylem and may act to influence the branch of phenylpropanoid metabolism involved in lignin production (Campbell et al., Plant Physiol. 108 (Suppl.), 28 (1995)).

A second reported role for plant MYB genes is in the control of cell shape. For example, the MIXTA gene of Antirrhinum and the homologue PhMYB1 gene from Petunia have been shown to play a role in the development of the conical form of petal epidermal cells and the GL1 gene of Arabidopsis has been shown to be essential for the differentiation of hair cells (trichomes) in some parts of the leaf and in the stem (Noda et al., Nature 369:661-664 (1994); Oppenheimer et al., Cell 67:483-493 (1991); Mur, Ph.D. Thesis, Vrije Univ. of Amsterdam (1995)). Overexpression of MIXTA in transgenic tobacco results in trichome formation on pedals, suggesting that conical petal cells might be ‘trichoblasts’ arrested at an early stage in trichome formation.

GLI of Arabidopsis is associated with the expansion in the size of the cell that develops into the trichome, and it acts upstream of a number of other genes (Huilskamp et al., Cell 76:555-566 (1994)). GLI mutants can exhibit cellular outgrowths that do not develop into full branched trichomes. GL2 of Arabidopsis encodes a homeodomain protein that is associated with chome development (Rerie et al., Genes Dev. 8: 1388-1399 (1994)). The GL2 gene promoter contains motifs very similar to the binding sites of P and AmMYB305 transcription factors (Rerie et al., Genes Dev. 8:1388-1399 (1994)).

The conical cells produced by the action of the MIXTA gene of Antirrhinum resemble the limited outgrowths produced in Arabidopsis g12 mutants where trichome formation is aborted. In its regulation of trichome formation, GLI interacts with the product of the TTG gene, which is required for trichome formation and anthocyanin production (Lloyd et al., Science 258:1773-1775 (1992)). Expression of the maize R gene complements the ttg mutation and it has been reported that the TTG gene product is also a R-related protein that interacts with GL1 in a matter analogous to the interaction of C1 and R in maize (Lloyd et al., Science 258:1773-1775 (1992)).

A further reported role for plant MYB proteins is in hormonal responses during seed development and germination. A barley MYB protein (GAMY) whose expression is induced by gibberellic acid (GA) has been shown to activate expression of a gene encoding a high pI α-amylase that is synthesized in barley aleurone upon germination for the mobilization of starch in the endosperm (Larkin et al., Plant Cell 5:1739-1748 (1993)). Expression of GAMYB is induced by treatment of aleurone layers with GA and expression of the α-amylase gene is induced subsequently. There is a suggestion that other GA-inducible genes can also respond to activation by MYB proteins during seed germination because MYB-like motifs from other GA-responsive gene promoters have been shown to direct reporter gene expression in response to GA (Larkin et al., Plant Cell 5:1739-1748 (1993)). In addition, some MYB genes are expressed in response to GA treatment of Petunia petals (Mur, Ph.D. Thesis, Vrije Univ. of Amsterdam (1995)).

Treatment with another plant hormone, abscisic acid (ABA), induces expression of AtMYB2 in Arabidopsis, a MYB gene that is also induced in response to dehydration or salt stress (Shinozaki et al., Plant Mol. 19:439-499 (1992)). In maize, expression of the C1 gene is also ABA-responsive, where it is involved in the formation of anthocyanin in the developing kernels (Larkin et al., Plant Cell 5:1739-1748 (1993)). The rd22 gene promoter contains MYC-recognition sequences suggesting that AtMYB2 can interact with a bHLH protein to induce gene transcription in response to dehydration or salt stress (Iwasaki et al., Mol. Gen. Genet. 247:391-398 (1995)).

Plant transcription factors that fall within the helix-loop-helix class of transcription factors have been reported. These include the transcription factor encoded by the maize R and B class gene (Radicella et al., Genes and Development 6:2152-2164 (1992)). Alleles that have been identified at the b and r loci show differences in developmental or tissue specific expression.

Homeodomain transcription factors have been isolated from different plant species (Ma et al., Plant. Molec. Biol. 24:465-473 (1994); Muller et al., Nature 374:727 (1995); Lincoln et al., Plant Cell 6:1859-1876 (1994); Hareven et al., Cell 84:735-744 (1996); Vollbrecht et al., Nature 350:241-243 (1991)).

The homeodomain contains three α-helices (Quain et al., Cell 59:573-580 (1989)). Residues in helix 3 contact the major groove of a nucleic acid in a sequence specific manner. Although structurally similar, different homeodomains are able to recognize diverse binding sites (Hanes et al., Cell 57:1275-1283 (1989); Treisamn et al., Genes Dev. 5:594-604 (1991); Affolter et al., Proc. Natl. Acad. Sci. (U.S.A.) 87:4093-4097 (1990); Percival-Smith et al., EMBO J. 9:3967-3974 (1990)).

One class of homeodomain transcription factors are those that share a conserved cysteine-rich motif as illustrated by the Arabidopsis GLABRA2 homeodomain protein and the maize KNOTTED1 (KN1)-like proteins (Vollbrecht et al., Nature 350:241-243 (1991), Ma et al., Plant. Molec. Biol. 24:465-473 (1994)). The morphological mutation Knotted1 in maize alters the developmental fate of cells in leaf blades with wild-type expression of the gene localized in the meristem and ground tissue but absent from leaves or leaf primordia (Hake, Trends in Genetics 8:109-114 (1992); Freeling and Hake, Genetics 111:617-634 (1995)). In addition to having a homeodomain, the kn1 class of genes in maize encode an ELK domain which contains repeating hydrophobic residues (Kerstetter et al., Plant Cell 6:1877-1887 (1994)).

Kn1-like homeodomain genes have been reported in other plants, such as Arabidopsis (Lincoln et al., Plant Cell 6:1859-1876 (1994)), tomato and soybean (Ma et al., Plant Molecular Biology 24:465-473 (1994)).

Homeodomain transcription factors have been associated with the regulation of cell to cell communication and development in plants. Presence of the KNOTTED1 homeodomain transcription factor in a plant cell can lead to an increase in plasmodesmal size permitting the transport of larger molecules between cells (Lucas et al., Science 270:1980-1983 (1995)).

Another class of transcription factors, the polycomb-like transcription factors, have been reported in plants (Goodrich et al., Nature 386:44-51 (1997)). Wild type CLF, a polycomb-like transcription factor, isolated from Arabidopsis, exhibits extensive structural homology with Drosphilia Pc-G genes plants (Goodrich et al., Nature 386:44-51 (1997)). Like Drosphila Pc-G genes, the CLF genes encodes for a SET domain and two cysteine rich regions. CLF, while not being necessary for initial specification of stamen and carpel development, is reportedly necessary to later stages of development plants and represses a second transcription factor AGAMOUS (Goodrich et al., Nature 386:44-51 (1997); Schumacher and Magnuson, Trends in Genetics 13(5):167-170 (1997)).

A further class of transcription factors, those containing an AP2 domain, a conserved motif first identified in Arabidopsis (a floral mutant), has been identified in a number of plants (Jofuka et al., Plant Cell 6:1211-1225 (1994); Weigal et al., Plant Cell 7:388-389 (1995)). The AP2 domain, which is a DNA-binding motif of about 60 amino acid has been reported, for example, to be present in the Arabidopsis transcription factors CBF1, APETALA2, AINTEGUMENTA, and TINY; as well as the tobacco ethylene response element binding proteins (Moose and Sisco, Genes and Development 10:3018-3027 (1996)). Weigal et al., reports a 24 amino acid AP2 consensus domain which is predicted to form an amphipathic α-helix that may mediate protein-protein interactions (Weigal et al., Plant Cell 7.388-389 (1995)).

Mutations of transcription factors containing an AP2 domain have been to effect floral and ovule development (Meyerowitz et al., Cell 88:299-308 (1997)). Other transcription factors from this family have been reported to play a role in cold- and dehydration-regulated gene expression (Stockinger et al., Proc. Natl. Acad. Sci. (U.S.A.) 94(3):035-1040 (1997)).

Zinc-finger proteins have been isolated from plants (Takatsuji and Matsumoto, J. Biol. Chem. 271:23368-23373 (1996); Messner, Plant Mol. Biol. 33:615-624 (1997); Dietrich et al., Cell 88:685-694 (1997); Pater et al., Nucleic Acid Research 24:4624-4631 (1996); Tague and Goodman, Plant Mole. Biol. 28:267-279 (1995); Putterill et al., Cell 80:847-857 (1995); Takatsuji et al., Plant Cell 6:947-958 (1994)). Zinc-finger proteins have been associated with a number of processes in plants including cell death (Dietrich et al., Cell 88:685-694 (1997)) and flower morphology (Pater et al., Nucleic Acid Research 24:462-44631 (1996)).

The term zinc-finger has been applied to a broad set of protein motifs. Zinc-finger transcription factors may be subdivided into a number of categories. A category of zinc-finger transcription factors referred to as the C₂H₂ zinc finger transcription factors (also referred to as either TFIIA or Krüpell-like zinc fingers) (Meissner and Michael, Plant Molecular Biology 33:615-624 (1997); Takatsuji et al., EMBO J. 11: 241-249 (1994); Tague and Goodman, Plant Mol. Biol. 28:267-279 (1995); Takasuji et al., Plant Cell 6:947-948 (1994), Sakamoto et al., Eur. J. Biochem. 217:1049-1056 (1993); Saki et al., Nature 378:199-203 (1995)). C₂H₂ zinc finger transcription factors have been reported, which contain one, two or three zinc fingers. These zinc fingers are maintained by cysteine and/or histidine residues organized around a zinc metal ion (Meissner and Michael, Plant Molecular Biology 33:615-624 (1997)).

Examples of C₂H₂ zinc finger transcription factors include: the petunia Epf1 product which binds to an inverted repeat found in the promoter of EPSP, the W2f1 product from wheat, which binds to a nonameric motif found in the histone H3 promoter; the Arabidopsis AtZFP1 product associated with shoot development; and the Arabidopsis SUPERMAN product that is associated with negative regulation of B-function floral organ identity (Meissner and Michael, Plant Molecular Biology 33:615-624 (1997); Takatsuji et al., EMBO J. 11:241-249 (1994); Tague and Goodman, Plant Mol. Biol. 28:267-279 (1995); Takasuji et al., Plant Cell 6:947-948 (1994), Sakamoto et al., Eur. J. Biochem. 217:1049-1056 (1993); Saki et al., Nature 378:199-203 (1995)).

Another category of zinc-finger transcription factor include plant relatives of the GATA-1 transcription factor (Dietrich et al., Cell 88: 685-694 (1997); Evans and Felsenfeld, Cell 58:877-885 (1989); Putterill et al., Cell 80:847-857 (1995); Yanagisawa et al., Nucleic Acid Research 23:3403-3410 (1995); De Paolis et al., Plant J. 10:215-224 (1996); Lippuner et al., J. Biol. Chem. 271:12859-12866 (1996)). GATA-1 like transcription factors have been associated with, for example, the regulation of cell death and the regulation of expression associated with salt stress.

6. R-Gene Products

Plant disease resistance is often a consequence of the induction of certain defense responses in the plant. One such defense response is the hypersensitive response (HR), which is induced in infected plant cells, resulting in cell wall depositions and localized cell death. The HR is reported to prevent pathogens from spreading throughout the plant by depriving the pathogen of a living host cell. From genetic analysis of plant-pathogen interactions, it has been reported that HR-associated resistance is dependent on the presence of active resistance genes (R-genes) in the plant and corresponding avirulence genes (avr) in the pathogen (Flor, Annu. Rev. Phytopathol. 28:275-296 (1971)). In yeast cells, it has been reported that an R-gene product and it's cognate avr gene product directly interact. In the absence of either of these genes, the interaction between plant and pathogen may result in disease. A second defense response, linked to R-gene triggered HR, results in the systemic induction of a number of host defense proteins and the establishment of systemic acquired resistance (SAR).

In addition to avr genes, hrp genes have been shown to be involved in the production and secretion of bacterial virulence/avirulence proteins, including harpin of Erwinia amylovora and harpinPss of Pseudomonas syringae (Gopolan et al., Plant J. 10:591-600 (1996)).

Isolated R-genes, or fragments thereof, have been used to identify R-gene homologues in plants (Kanazin et al., Proc. Natl. Acad. Sci. (U.S.A.) 93:11746-11750 (1996); Leister et al., Nature Genetic 14:421-429 (1996)). A number of R-gene homologues have been identified by Southern analysis using R-gene probes. Other R-genes have been identified and isolated using primers that anneal to the conserved regions of R-genes (Staskawicz et al., Science 268:661-667 (1995)).

It has also been reported that HR can be induced by overexpression of R-genes. Tobacco elicitor-binding proteins and tobacco genes with unknown function have also been reported to induce or enhance HR (Karrer et al., unpublished sequences U66265-U66273 (1996)). An early event in HR is reported to be the rapid production of reactive oxygen products such as hydrogen peroxide. The enhancement of hydrogen peroxide production by overexpression of certain enzymes such as glucose oxidase from Aspergillus niger has been reported to result in increased resistance to certain pathogens (Wu et al., Plant Cell 7:1357-1368 (1995)).

It has been reported that cloned R-genes can be transferred from resistant plant species to susceptible plant species. For example, R-genes have been transferred from tomato to tobacco (Rommens et al., Plant Cell 7:1537-1544 (1995); Thilmony et al., Plant Cell 7:1529-1536 (1995)), or tobacco to tomato (Whitham et al., Proc. Natl. Acad. Sci. (U.S.A.) 93:8776-8781 (1996)).

R-genes involved in HR have been isolated. Sequence comparisons group R-gene products into four different classes. Class I R-genes encode products that can be characterized by the presence of a leucine rich repeat, a nucleotide binding site and a stretch of amino acids with the consensus sequence “GLPLAL”. R-genes that belong to this class include: Arabidopsis Rps2, Arabidopsis Rpm1, Arabidopsis Rpp5, tomato Prf, tomato I2, flax L6, flax M, tobacco N, and wheat Cre3 (Hammond-Kosack and Jones, Plant Cell 8:1773-1791 (1996)). Other reported members of this class include tomato Mi and potato Rx1. The class I R-gene products can be further divided into two subclasses that are characterized by either (A) the presence of an N-terminal leucine zipper (e.g., Rps2 and Rpm1), or (B) the presence of a Drosophila Toll/Human interleukin-1 cytoplasmic like domain (e.g., L6, tobacco N and Rpp5). For example, 12, an R-gene product that contains a leucine zipper, confers Lycopersicon esculentum resistance against the fungus Fusarium oxysporum f. sp. lycopersici race 2 in tomato (Segal et al., Mol. Gen. Genet. 231:179-185 (1992)).

An L6 rust resistance gene from flax has been cloned after tagging with the maize transposable element Activator (Lawrence et al., Plant Cell 7:1195-1206 (1995); Lawrence et al., Plant J. 4:659-669 (1993). An M rust resistance gene has been cloned from flax (Anderson et al., Plant Cell 9:641-651 (1997)). The cloned M rust resistance gene encodes a protein of the nucleotide binding site leucine-rich repeat class and is related to the unlinked L6 rust resistance gene (86% nucleotide identity).

A N gene of tobacco mediates resistance to the pathogen tobacco mosaic virus (TMV) (Dinesh-Kumar et al., Proc. Natl. Acad. Sci. (U.S.A.) 92:4175-4180 (1995)). It has been reported that N confers a hypersensitive response and effectively localizes tobacco mosaic virus to the site of inoculation in transgenic tomato, as it does in tobacco. An N gene has been isolated by transposon tagging using the maize Activator transposon (Whitham et al., Cell 78:1101-1115 (1994)). Transgenic tomato plants bearing an N gene have been reported (Whitham et al., Proc. Natl. Acad. Sci. (U.S.A.) 93:8776-8781 (1996)).

Other characterized Class I R-genes are reported to contain a leucine-rich repeat. This leucine rich repeat has been implicated in protein-protein interactions, and it has been reported that it may confer specificity on the R-gene product. The leucine rich repeat is conserved in R-gene proteins. Two mutant proteins of Rps2 and Rpm1 are reported to be nonfunctional because of a single amino change within the leucine rich repeat (Bent et al., Science 265:1856-1860 (1994); Grant et al., Science 269:843-846 (1995)). Plant galacturonase inhibitors (PGIs) contain leucine rich repeats that share homology with the leucine repeats of R-genes. PGIs are induced by plant pathogens and can trigger plant defense responses (Bergmann et al., Plant J. 5:625-634 (1994)). It has been reported that the mode of action of PGIs is similar to that of R-genes.

Plant with inactivated genes that function downstream from the point where different R-gene cascades merge are not reported to induce spontaneous lesions and are not reported to affect a plant's ability to generate HR to avirulent pathogens. However, reported mutations in these genes result in a loss of specific resistances to certain pathogens. Examples of these genes are the Arabidopsis genes Ndr1 (Century et al., Proc. Natl. Acad. Sci. (U.S.A.) 92:6597-6601 (1995)) and Eds1 (Parker et al., Plant Cell 8:2033-2046 (1996)). R-gene products that trigger the Ndr1 pathway often contain, apart from a leucine rich repeat and a nucleotide binding site, a leucine zipper (e.g., Rpm1, Rps2). A tobacco protein, Hin1, is reported to share homology with Ndr1 and to be upregulated by pathogen elicitors (Gopalan et al., Plant J. 10:591-600 (1996)). It has been reported that Hin1 may have a similar role in plant defense as Ndr1.

A Eds1 pathway can be activated by R-gene products that lack a leucine zipper (e.g., Rpp5). An Arabidopsis Rpp5 gene specifying resistance to the downy mildew pathogen Peronospora parasitica has been positionally cloned and encodes a protein that possesses a putative nucleotide binding site and leucine-rich repeats and exhibits structural similarity to the plant resistance gene products N and L6 (Parker et al., Plant Cell. 9:879-894 (1997); Reignault et al., Mol. Plant. Microbe Interact. 9:464-473 (1996)).

Class II R-gene products contain a leucine rich repeat and a putative conserved membrane anchor. Members of this class include the tomato genes Cf2, Cf4, Cf5, and Cf9 (Hammond-Kosack and Jones, Plant Cell 8:1773-1791 (1996)). Tomato Cf genes confer resistance to C. fulvum. These genes reside in complex loci and encode predicted membrane-bound proteins with extracytoplasmic leucine-rich repeats (Parniske et al., Cell 91:821-832 (1997)). Cf4, which also encodes a membrane-anchored extracellular glycoprotein, has been cloned and characterized (Thomas et al., Plant Cell 9:2209-2224 (1997)). Cf4 contains 25 leucine-rich repeats, which is two fewer than the number of leucine rich repeats in Cf9. The Cf9 resistance gene encodes a membrane-anchored extracellular glycoprotein that contains leucine-rich repeats (de Wit et al., Antonie Van Leeuwenhoek 71:137-141 (1997)). A Cf9 gene has been isolated by transposon tagging with the maize transposable element Dissociation (Jones et al., Science 266:789-793 (1994)). It has been reported that the avr gene product avr9 of Cladosporium fulvum, which functions as an elicitor of the tomato resistance response, binds to cell walls of both resistant Cf9/Cf9 and susceptible cf9/cf9 plants (Kooman-Gersmann et al., Plant Cell 8:929-938 (1996)).

A tomato resistance locus Cf2 has been isolated by positional cloning and is reported to contain two almost identical genes, each conferring resistance to isolates of tomato leaf mold (C. fulvum) expressing the corresponding Avr2 gene (Dixon et al., Cell 84:451-459 (1996)). These two Cf2 genes encode protein products that differ from each other by only three amino acids and contain 38 leucine-rich repeat motifs. In the two reported Cf2 genes, 20 of the leucine-rich repeat motifs are reported to contain conserved alternating repeats. The C-terminus of Cf2 carries regions of pronounced homology to the protein encoded by the unlinked Cf9 gene.

A third class of R-gene products consists of serine/threonine protein kinases which share homology to the human interleukin-1 receptor associated protein kinase, IRAK, that is essential for the activation of the transcription factor NF-kB (Cao et al., Science 271:1128-1131 (1996)). Class III R-genes include Pto and Lrk10 (Feuillet et al., Plant J. 11:45-52 (1997)). Pto, which lacks a leucine rich repeat, is reported to interact, directly or indirectly, with another protein in the Pto pathway, Pseudomonas resistance and fenthion sensitivity (Prf) protein, which contains a leucine rich repeat (Salmeron et al., Cell 86:123-133 (1996); Salmeron et al., Plant Cell 6:511-520 (1994)). Prf also contains leucine-zipper, and nucleotide-binding motifs (Salmeron et al., Cell 86:123-133 (1996)). Lrk10, which encodes a receptor-like protein kinase, has been isolated from wheat by screening a set of near-isogenic lines carrying different leaf rust resistance genes with a wheat probe encoding a serine/threonine protein kinase (Feuillet et al., Plant J. 11:45-52 (1997)).

A tomato serine/threonine protein kinase, Pto, binds to the avrPto protein of Pseudomonas syringae pv. tomato and functions as an elicitor receptor (Tang et al., Science 274:2060-2063 (1996)). The Pto gene encodes a serine/threonine protein kinase with an N-terminal myristoylation site. Pto is reported to play a role in signaling and membrane targeting (Martin et al., Science 262:1432-1436 (1993)). Pto is reported to exhibit similarity to SRK6, which is associated with cell recognition during pollination.

The protein kinase encoded by Pto has been shown to bind to a number of different proteins in vivo are reported to be associated with different aspects of the defense pathway. Pto physically interacts with a second IRAK-like kinase, Pti1 (Pto interacting protein). Pti1 is reported to function downstream of Pto in a kinase cascade and has been implicated in the pathway leading to HR cell death (Zhou et al., Cell 83:925-935 (1995)). Pto has also been reported to interact with a second class of proteins which includes, for example, Pti4, Pti5, and Pti6. The second class of proteins is reported to operate in the pathway leading to activation of defense proteins and SAR (Zhou et al., EMBO J. 16:3207-3218 (1997)). On the basis of sequence homology and DNA binding studies, Pti4, Pti5, and Pti6 are reported to be associated with the transcriptional activation of genes encoding defense proteins, called pathogenesis related (PR) genes, that play a role in establishing SAR.

A fourth class of R-genes encode receptor kinases having an extracellular leucine rich repeat and a cytoplasmic kinase domain. The fourth class of R-genes include, for example, the rice gene Xa21 (Hammond-Kosack and Jones, Plant Cell 8:1773-1791 (1996)). The rice gene Xa21, which confers resistance to Xanthomonas oryzae pv. oryzae (Xoo), has been isolated using a map-based cloning strategy (Ronald, Plant Mol. Biol. 35:179-186 (1997); Wang et al., Mol. Plant Microbe Interact. 9:850-855 (1996)). The protein has both a leucine-rich repeat motif and a serine-threonine kinase-like domain (Song et al., Science 270:1804-1806 (1995)). A putative receptor kinase with homology to Xa21 is the Brassica protein SFR2, which has been implicated in the autoincompatibility response that leads to rejection of self pollen. Thus, both Xa21 and SRF2 are reported to be involved in cell-cell interactions. It has been reported that SFR2 mRNA accumulates in response to infiltration with bacteria and salicylic acid (Pastuglia et al., Plant Cell 9:49-60 (1997)).

A fifth class of R-genes encodes for proteins with a leucine rich repeat-like structure. The fifth class of R-genes include, for example, the sugar beet Hs1 gene (Cai et al., Science 275:832-34 (1997)). Hs1 is reported to confer resistance to the beet cyst nematode (Heterodera schachtii Schmidt).

Systemic acquired resistance is linked to R-gene defense, but extends host “immunity” beyond the primary site of pathogen attack. SAR is triggered at infection sites undergoing HR, and becomes systemically established, often over the course of about one week. Since many pathogenesis related (PR) proteins synthesized during this response display antimicrobial activity, SAR is associated with broad-spectrum control of many biotrophic pathogens.

Mutations that lead to spontaneous formation of HR lesions and constitutive expression of SAR proteins have identified genes associated with HR and SAR. Examples of such genes include in Arabidopsis Cpr5 (Bowling et al., Plant Cell 9:1573-1584 (1997)), Acd2 (Greenberg et al., Cell 7:551-63 (1994)), and Lsd1 (Dietrich et al., Cell 88:685-94 (1997)). The Lsd1 gene encodes a protein with three zinc finger domains, suggesting a role in transcriptional regulation. Lsd1 mutants are hyper-responsive to cell death and fail to limit the extent of death regulation (Dietrich et al., Cell 88:685-694 (1997)).

Inactivation of genes functioning downstream of the HR/SAR branchpoint, such as the Arabidopsis Cpr1, Cpr6, Cim2 and Cim3 genes, result in constitutive expression of SAR (Bowling et al., Plant Cell 6:1845-1857 (1994); Ryals et al., Plant Cell 8:1809-1819 (1996)).

The activating function of mutant genes on SAR is correlated with increased salicylic acid (SA) levels (Ryals et al., Plant Cell 8:1809-1819 (1996)). It has been reported that SA plays a role in both SAR signaling and disease resistance. An increased SA level is associated with SAR in tobacco and cucumber, as well as other plant species. Depletion of SA has been reported to be associated with the breakdown of SAR in tobacco and Arabidopsis (Ryals et al., Plant Cell 8:1809-1819 (1996)). SA is a product of phenylpropanoid metabolism formed via decarboxylation of trans-cinnamic acid to benzoic acid and its subsequent 2-hydroxylation to SA. Newly synthesized SA is rapidly metabolized to SA O-beta-D-glucoside and methyl salicylate. Two enzymes involved in SA biosynthesis and metabolism are benzoic acid 2-hydroxylase (BA2H), which converts benzoic acid to SA, and UDPglucose:SA glucosyltransferase (SA GTase (EC 2.4.1.35)), which catalyzes the conversion of SA to SA glucoside.

A BA2H enzyme has been partially purified and characterized as a soluble protein of 160 kDa that belongs to a class of cytochrome P450 monoxygenases (Leon et al., Proc. Natl. Acad. Sci. (U.S.A.) 92:10413-10417 (1995)). Pathogen infection has been associated with increases in BA2H levels that parallel free SA levels. BA2H has been reported to have a regulatory role in SA accumulation during the development of SAR (Leon et al., Plant Phys. 103:323-28 (1993)).

Another group of proteins with a function in the plant pathogen-response are mitogen-activated protein kinases (MAPKs) that share structural and/or functional homology to the mammalian stress-activated protein kinase, SAPK. SAPK is the dominant c-Jun amino-terminal protein kinase activated in response to cellular stress, such as treatment with tumor-necrosis factor-alpha and interleukin-beta (Sanchez et al., Nature 372:794-798 (1994)). In tobacco, SA was shown to induce a rapid and transient activation of a 48 kD MAPK called p48 SIP kinase (for SA-Induced Protein kinase). Biologically active analogs of SA, which induce pathogenesis-related genes and enhanced resistance, also activated this kinase. The SIP kinase is phosphorylated on a tyrosine residue(s), and treatment with either tyrosine or serine/threonine phosphatases abolished its activity. Analysis of the SIP kinase sequence indicates that it belongs to the MAP kinase family and that it is distinct from the other plant MAP kinases previously implicated in stress responses (Zhang and Klessig, Plant Cell 9:809-824 (1995)). Another MAPK has been reported to accumulate one minute after mechanical wounding in tobacco. Inactivation of the endogenous homologous gene resulted in the inhibition of both wound-induced gene transcription and biosynthesis of jasmonic acid (Seo et al., Science 270:1988-1992 (1995)).

An Arabidopsis Npr1 gene is reported to act downstream of SA. Mutants that contain an inactivated Npr1 gene accumulate high levels of SA, but are unable to establish SAR. The Arabidopsis Npr1 gene controls the onset of systemic acquired resistance (SAR), a plant immunity, to a broad spectrum of pathogens that is normally established after a primary exposure to avirulent pathogens (Cao et al., Cell 88:57-63 (1997)). Mutants with defects in Npr1 fail to respond to various SAR-inducing treatments and display limited expression of PR genes and exhibit increased susceptibility to infections.

An Npr1 gene has been cloned from Arabidopsis (Cao et al., Cell 88:57-63 (1997)). Npr1 mutants are susceptible to a variety of pathogens. Npr1 may function in the signal transduction pathway after the specific R-gene pathways converge. Overexpression of Npr1 protein in Arabidopsis is reported to lead to a faster defense response and heightened resistance to both fungal and bacterial pathogens. Protein motifs in Npr1 include ankyrin repeats that have been reported to mediate protein-protein interactions, and putative nuclear localization signals.

The Npr1 protein has been reported to have a regulatory role as a transcriptional activator of defense genes. A Npr1/green fluorescent protein (GFP) fusion has been reported to restore wild type Npr1 activity in Arabidopsis mutants. After challenge with an avirulent pathogen or chemical elicitor, the Npr1-GFP fusion is translocated from the cytoplasm to the nucleus of the challenged plant cells. Other putative transcription factors that may play a role in the activation of plant defense genes are the tomato proteins Pti4, Pti5 and Pti6. These proteins contain an ethylene-responsive element-binding domain, EREBP, and bind to several promoters of pathogenesis-related genes (Zhou et al., EMBO J. 16:3207-3218 (1997)). Also, the parsley elicitor-inducible DNA binding proteins BPF1, which binds to the P box of certain defense related promoters (da Costa e Silva et al., Plant J. 4:125-135 (1993)), and WRKY1-WRKY3, which bind to the promoter of PR1 (Rushton et al., EMBO J. 15:5690-5700 (1996)), are reported to function in plant defense responses. Furthermore, heat stress-inducible transcription factors hsf8 and hsf30 (Treuter et al., Mol. Gen. Genet. 240:113-125 (1993)) and hsfA1 and hsfA2 (Boscheinen et al., Mol. Gen. Genet. 255:322-331 (1997) are reported to play a role in the plant stress and pathogen response.

Additional pathways in the downstream defense response signaling events have been reported in plants. Inactivation of the Arabidopsis Cpr6 gene results in constitutive expression of PR genes that are SA-dependent and independent of the presence of a functional Npr1 gene (Clark et al., Plant Cell, in press). In addition, Cpr6 (and Cpr5) inactivation results in constitutive expression of defense genes such as plant defensin PDF1.2 and plant thionin THI2.1 genes, which in wild type plants, are induced by an SAR pathway in a manner that is independent from SA (Bowling et al., Plant Cell 9:1573-1584 (1997); Penninckx et al., Plant Cell 8:2309-23 (1996)). This SA-independent SAR pathway can be activated by infection of plants with certain necrotrophic pathogens such as Fusarium oxysporum (Epple et al., Plant Cell 9:509-520 (1997)), and Alternaria brassicicola (Penninckx et al., Plant Cell 8:2309-2323 (1996)). Chemical agents such as jasmonic acid and coronatin can induce this pathway leading to acquired resistance against both necrotrophic pathogens and biotrophic pathogens, such as Peronospora parasitica (Bowling et al., Plant Cell 9:1573-1584 (1997); Cao et al., Cell 88:57-63 (1997)). SA has been reported to inhibit jasmonic acid (JA) biosynthesis (Pena-Cortes et al., Biochem. Soc. Symp. 60:143-148 (1994)) and high SA levels may block the induction of defense genes effective against certain necrotrophic pathogens.

Another gene, PAD4, reported to play a role in both SAR and SA-independent SAR pathway, is involved in accumulation of SA and biosynthesis of the phytoalexin calmodulin. Inactivation of PAD4 is reported to cause sensitivity to downy mildews (Glazebrook et al., Genetics 146:381-392 (1997)).

Phytoalexin production, which can be triggered by SA-independent SAR, can be decreased by, e.g., suppression of phenylalanine ammonium lyase (PAL) or by expression of tryptophan decarboxylase. Transgenic plants generated in this way have been reported to display an enhanced sensitivity to Cercospora nicotianae and Phytophthora infestans (Dixon and Paiva, Plant Cell 7:1085-1097 (1995)). Expression of a grapevine stilbene synthase (SS) gene in tobacco resulted in synthesis of the stilbene phytoalexin resveratrol and increased resistance to Botrytis cinerea (Hain et al., Nature 361:153-156 (1993)).

Another class of proteins with a function in phytoalexin production comprise proteins in the tryptophan pathway including anthranilate synthase alpha, anthranilate synthase beta, phosphoribosyl anthranilate transferase (PAT), tryptophan synthase beta, phosphoribosyl anthranilate isomerase and tryptophan synthase alpha, all of which are pathogen-inducible (Zhao and Last, Plant Cell 8:2235-2244 (1996)). Enzymes active in other phytoalexin biosynthesis pathways are 3-deoxy-D-arbino heptulosonate-7-phosphate synthase (shikimate pathway), which is induced by pathogens (Zhao and Last, Plant Cell 8:2235-2244 (1996)), and the elicitor-inducible tobacco enzymes 3-hydroxy-3-methylglutaryl-CoA-reductase, sesquiterpene cyclase and sesquiterpene capsidiol (Chappel and Noble, Plant Phys. 85:467-473 (1987); Vogeli and Chappel, Plant Phys. 88:1291-1296 (1988)).

Additional pathogen- and/or elicitor inducible enzymes with a function in phytoalexin biosynthesis are tobacco 5-epi-aristolchene synthase (Facchini et al., Proc. Natl. Acad. Sci. (U.S.A.) 89:11088-11092 (1992)), Medicago 6-phosphogluconate dehydrogenase (Fahrendorf et al., Plant Mol. Biol. 28:885-990 (1995)), carnation benzoyl CoA:anthranilate N benzoyltransferase (Yang et al., Plant Mol. Biol. 35:777-789 (1997)), cotton delta cadinine synthase (Chen et al., Arch. Biochem. Biophys. 324:255-266 (1995)), alfalfa isoflavone reductase (Paiva et al., Plant Mol. Biol. 17:653-657 (1991)), NAD(P)H dependent 6′-deoxychalcone synthase (Welle et al., Eur. J. Biochem. 196:423-430 (1991)), Glycyrrhiza polyketide reductase (Plant Phys. 111:347-348 (1996)), and Vitis sesquiterpene synthase (Garcia-Espana et al., Arch. Biochem. Biophys. 288:414-420 (1991)).

Phenylalanine ammonia-lyase (PAL (EC 4.3.1.5)) catalyzes the first reported reaction in the general phenylpropanoid pathway leading to the production of phenolic compounds which exhibit a range of biological function (Fukasawa-Akada et al., Plant Mol. Biol. 30:711-722 (1996)). PAL transcript levels are reported to be higher in flowers and roots than in leaves and stems of mature plants. PAL transcripts accumulate differentially during flower and leaf maturation. PAL mRNA levels decline during flower maturation but increase during leaf maturation. In leaves, PAL transcripts rapidly accumulate after wounding. Clones for phenylalanine ammonia-lyase (PAL) have been reported in maize, rice (Oryza sativa L.), Stylosanthes humilis, parsley, Arabidopsis thaliana, tobacco, (Nicotiana tabacum L. cv Samsun NN), and soybean (Rosler et al., Plant Physiol 113:175-179 (1997); Zhu et al., Plant Mol. Biol. 29:535-550 (1995); Manners et al., Plant Physiol. 108:1301-1302 (1995); Logemann et al., Proc. Natl. Acad. Sci. (U.S.A.) 92:5905-5909 (1995); Wanner et al., Plant Mol. Biol. 27:327-338 (1995); Pellegrini et al., Plant Physiol. 106:877-886 (1994); Frank and Vodkin, DNA Seq. 1:335-346 (1991)).

Lignin is a phenolic polymer based on cinnamyl alcohol subunits derived from phenyl propanoid metabolism. Lignin is reported to play a role in plant defense by strengthening cell walls (Dougles, Trends Plant Sci. 1:171-178 (1996)). Lignin biosynthesis includes hydroxylation reactions catalyzed by cinnamate-4-hydroxylase (C4H) and ferulate-5-hydroxylase (F5H) and methylation reactions catalyzed by the bispecific caffeic acid/5-hydroxyferulic acid O-methyltransferase (COMT). Reduction of ferulic acid and sinapic acid to their corresponding alcohols is reported to occur in three steps: formation of activated thioesters by the action of 4-coumarate:coenzyme A ligase (4CL), and reduction to the aldehydes and alcohols by the action of cinnamyl-CoA reductase and cinnamyl alcohol dehydrogenase (CAD (E.C. 1.1.1.195)), respectively. Alternatively, methylation can occur at the level of the CoA esters, such as 5-hydroxy-feruloyl-CoA, rather than that of the free acids by the action of caffeoyl-CoA O-methyltransferase (CCoOMT), or by O-methyltransferases on hydroxycinnamyl aldehydes or alcohols (Lee et al., Plant Cell 9:1985-1998 (1997); Grima-Pettenati et al., Phytochemistry 37:941-947 (1994); Grima-Pettenati et al., Plant Mol. Biol. 21:1085-1095 (1993)). CAD genes are expressed in response to different developmental and environmental cues (MacKay et al., Mol. Gen. Genet. 247:537-545 (1995)). Clones of CAD have been reported from the loblolly pine (Pinus taeda L.), Arabidopsis thaliana, Eucalyptus botryoides, and tobacco (MacKay et al., Mol. Gen. Genet. 247:537-545 (1995); Sommers et al., Plant Physiol. 108:1309-1310 (1995); Hibino et al., Plant Physiol. 104:305-306 (1994); Knight et al., Plant Mol. Biol. 19:793-801 (1992)). A bean CAD gene is reported to be share homology with a maize malic enzyme (Walter et al., Plant Mol. Biol. 15:525-526 (1990)).

Cinnamate 4-hydroxylase (C4H) is the first reported Cyst P450-dependent monooxygenase of the phenylpropanoif pathway (Bell-Lelong et al., Plant Physiol. 113:729-738 (1997)). A cDNA for C4H has been isolated from Arabidopsis thaliana using a C4H cDNA from mung bean as a hybridization probe (Mizutani et al., Plant Physiol. 113:755-763 (1997)).

Ferulate-5-hydroxylase (F5H) is a cytochrome P450-dependent monooxygenase (P450) of the general phenylpropanoid pathway (Meyer et al., Proc. Natl. Acad. Sci. (U.S.A.) 93:6869-6874 (1996)).

Caffeoyl-CoA 3-O-methyltransferase (CCoAOMT) cDNA clones have been isolated from RNA extracted from TMV-infected tobacco leaves (Martz et al., Plant Mol. Biol. 36:427-437 (1998)). Two members of the CCoAOMT gene family are reported to be constitutively expressed in plant organs and tissues whereas another two members are reported to be preferentially expressed in flower organs, after tobacco mosaic virus (TMV) infection or elicitor treatment of leaves.

Caffeic acid O-methyltransferase (COMT (EC 2.1.1.6)), which is associated with lignin biosynthesis, exists as an active monomer of subunit molecular weight of 41,000 daltons (Vignols et al., Plant Cell 7:407-416 (1995)). The pattern of expression that results from the use of a COMT gene zea mays gene has been studied by histochemical and fluorometric beta-glucuronidase (GUS) analysis in transgenic maize and tobacco plants (Capellades et al., Plant Mol. Biol. 31:307-322 (1996)). This COMT promoter directs GUS expression to the xylem and the other tissues undergoing lignification, and it is unregulated in respond to wounding and to elicitors. COMT can be separated into two forms on the basis of its isoelectric points and relative affinities for S-adenosyl-methionine and S-adenosylhomocysteine (Edwards and Dixon, Arch Biochem Biophys 287:372-379 (1991)).

The phenylpropanoid enzyme 4-coumarate:coenzyme A ligase (4CL (EC 6.2.1.12)) is reported to activate the hydroxycinnamic acids for the biosynthesis of the coniferyl and sinapyl alcohols that are subsequently polymerized into lignin (Lee et al., Plant Cell 9:1985-1998 (1997)). 4CL clones have been reported from loblolly pine (Pinus taeda L.) (Zhang and Chiang, Plant Physiol 113:65-74 (1997)), Glycine max (Uhlmann and Ebel, Plant Physiol. 102:1147-1156 (1993)), Lithospermum erythrorhizon (Yazaki et al., Plant Cell Physiol. 36:1319-1329 (1995)), Arabidopsis thaliana (Lee et al., Plant Mol. Biol. 28:871-884 (1995)), and tobacco (Lee and Douglas, Plant Physiol 112:193-205 (1996)). 4CL antisense lines have been reported in Arabidopsis and tobacco (Kajita et al., Plant Cell Physiol. 37:957-965 (1996).

4CL has been reported to catalyze the activation of 4-coumaric acid but benzoic acids (Barillas and Beerhues, Planta 202:112-116 (1997)). 4CL expression is activated early during seedling development and has been reported to be correlated with the onset of lignin deposition in cotyledons and roots 2-3 days after germination. mRNA accumulation is transiently activated by wounding of mature Arabidopsis leaves. 4CL has been purified from differentiating xylem of loblolly pine (Pinus taeda L.) and cambial sap of spruce (Picea abies). (Luderitz et al., Eur J Biochem 123:583-586 (1982)). The pine 4CL has been reported to have an apparent molecular mass of 64 kD (Voo et al., Plant Physiol. 108:85-97 (1995)). Ferulic, 4-coumaric and caffeic acids are reported to be substrates for 4CL.

Other enzymes that are involved in cell wall strengthening are elicitor- and/or pathogen inducible peroxidases, isolated from, e.g., Medicago (Cook et al., Plant Cell 7:43-55 (1995)) and tomato (Vera et al., Mol. Plant. Microbe Interact. 6:790-794 (1993)). In addition, wound- or pathogen inducible hydroxyproline-rich proteins have been isolated from plants such as pea (Banik et al., Plant Mol. Biol. 31:1163-1172 (1996)) and sunflower.

Plant defense response can also limit damage caused by oxidative stress and/or cell death. Enzymes that play a role in protection against free radicals include, but are not limited to, glutathione-S-transferase (Dudler et al., Mol. Plant-Microbe Interact. 4:14-18 (1991)), blue copper protein and iron superoxide dismutase. DAD1 is a human protein that is reported to play a role in preventing programmed cell death.

An early plant signal reported to be involved in the induction of defense responses is a tomato 18-amino acid polypeptide systemin. This polypeptide is reported to activate defense genes at levels as low as fmols/plant. As with animal polypeptide hormones, systemin is derived from a larger precursor protein, called prosystemin, by limited proteolysis. Systemin has been reported, by autoradiography experiments, to be phloem mobile and, by antisense experiments, to be a component of the wound-inducible, systemic signal transduction system leading to the transcriptional activation of the defensive genes (Schaller and Ryan, Bioessays 18:27-33 (1996)). It has also been reported that systemin is a wound hormone.

Other early signals for plant defense responses are salicylic acid and jasmonic acid. Several enzymes are reported to play a role in jasmonic acid biosynthesis. One of these enzymes is lipoxygenase (LOX (EC 1.13.11.12)). LOX is also reported to induce tuberization in potato (Royo et al., J. Biol. Chem. 271:21012-21019 (1996)). Purified soybean plasma membranes exhibit lipoxygenase activity with a pH optimum of 5.5-6.0 and a Kₘ value of 200 μM for both linolenic and linoleic acids (Macri et al., Biochim. Biophys. Acta 1215:109-114 (1994)). Lipoxygenase (LOX) clones have been reported from maize embryos (Jensen et al., Plant Mol. Biol. 33:605-614 (1997)), rice (Ohta et al., Eur. J. Biochem. 206:331-336 (1992)), tobacco (Veronesi et al., Plant Physiol. 112:997-1004 (1996)), cucumber seeds (Hohne et al., Eur. J. Biochem. 241:6-11 (1996)), potato (Royo et al., J. Biol. Chem. 271:21012-21019 (1996)), soybean (Saravitz and Siedow, Plant Physiol. 110:287-299 (1996)), and Arabidopsis (Melan et al., Biochim. Biophys. Acta 1210:377-380 (1994); Bell and Mullet, Plant Physiol. 103:1133-1137 (1993)).

LOX is a non-heme iron containing enzyme that catalyzes the formation of hydroperoxylinolenate from linolenic. Multiple forms of LOX are found in plants. Different LOX forms are reported to vary in their organ and tissue distribution and also differ in their substrate specificity and production of hydroperoxylinolenate isomers. LOX is generally found in multiple subcellular compartments. Plant LOX produces two linolenic acid derived isomers, 9-hydroperoxylinolenate and 13-hydroperoxylinolenate. 13-hydroperoxylinolenate is reported to be a precursor to jasmonic acid. An enzyme catalyzing the formation of 13-hydroperoxylinolenate for inducible jasmonic acid production is located in the plastid (Creelman and Mullet, Ann. Rev. Plant Physiol. Plant Mol. Biol. 48:335-381 (1997)). A rice LOX, with a plastid transit sequence has been reported (Peng et al., J. Biol. Chem. 269:3755-3761 (1994)). Transgenic Arabidopsis with a reduced accumulation of plastidic LOX2 failed to produce jasmonic acid in response to wounding (Bell et al., Proc. Natl. Acad. Sci. (U.S.A.) 92:8675-8679 (1995)). Non-plastidic LOX may play a role in constitutive production of jasmonic acid.

A second enzyme reported to be involved in jasmonic acid biosynthesis is allene oxide synthase (AOS (EC 4.2.1.92)), which converts 13-hydroperoxylinolenate to 12,13-epoxy-octadecatrienoic acid. Allene oxide synthase has been cloned from flax (Song et al., Proc. Natl. Acad. of Sci. (U.S.A.) 90:8519-8523 (1993)), guayule (Pan et al., J. Biol. Chem. 270:8487-8494 (1995)), and Arabidopsis (Laudert et al., Plant Molecular Biology 31:323-335 (1996)). AOS is a cytochrome P450 enzyme with a heme binding region in the C-terminal portion of the protein. Both the flax and Arabidopsis reported clones encode proteins with plastid transit sequences (Song et al., Proc. Natl. Acad. Sci. (U.S.A.) 90:8519-8523 (1993); Laudert et al., Plant Molecular Biology 31:323-335 (1996)). The flax clone has been expressed in potato, and the AOS protein was reported to accumulate in plastids. Transgenic potato plants expressing flax AOS are reported to have elevated levels of jasmonic acid. These elevated jasmonic acid levels did not result in constitutive expression of wound induced transcripts (Harms et al., Plant Cell 7:1645-1654 (1995)). The cloned Arabidopsis AOS gene has been expressed in E. coli, and is reported to accept either 13-hydroperoxylinolenate or 13-hydroperxoylinoleate as substrates (Laudert et al., Plant Molecular Biology 31:323-335 (1996)). The cloned guayule AOS is homologous to clones from Arabidopsis and flax and is associated with rubber particles in the bark parenchyma (Pan et al., J. Biol. Chem. 270:8487-8494 (1995)).

The epoxide product of AOS, 12,13-epoxy-octadecatrienoic acid, is converted to the enantiomerically pure 12-oxo-phytodienoic acid by the action of allene oxide cyclase (AOC (EC 5.3.99.6)). Activity of AOC been reported in the seed coat of immature soybean seeds (Simpson and Gardner, Plant Physiology 108:199-202 (1995)) and dry maize seeds (Ziegler et al., Plant Physiology 114:565-573 (1997)). The maize enzyme is reported to be soluble, with an approximate molecular weight of 45-47 kD. It is further reported that the maize enzyme is specific for 12,13-epoxy-octadecatrienoic acid derived from linolenic acid, and exhibits little activity with 12,13-epoxy-octadecadienoic acid, which is derived from linoleic acid (Ziegler et al., Plant Physiology 114:565-573 (1997)).

Mutation-induced recessive alleles (mlo) of the barley Mlo locus confer a leaf lesion phenotype and broad spectrum resistance to the fungal pathogen, Erysiphe graminis f. sp. hordei. A Mlo gene has been isolated from barley using a positional cloning approach (Buschges et al., Cell 88:695-705 (1997)). It has been reported that expression of mlo is restricted to a subcellular, highly localized cell wall apposition site directly beneath the site of abortive fungal penetration (Wolter et al., Mol. Gen. Genet. 239:122-128 (1993)). The deduced 60 kDa protein is reported to be membrane-anchored by at least six membrane-spanning helices. It has also been reported that mlo exhibits a dual negative control function of the Mlo protein in leaf cell death and in the onset of pathogen defense. It has also been reported that the absence of Mlo primes the responsiveness for the onset of multiple defense functions.

Chalcone synthase (EC 2.3.1.74) is an enzyme that catalyses the first reported reaction dedicated to the flavonoid pathway in higher plants. Chalcone synthase provides the C₁₋₅ chalcone intermediates from which other flavonoids originate by catalyzing the condensation of three molecules of malonyl-CoA with 4-coumaroyl-CoA. The chalcone synthase reaction results in the formation of 2′,4′,6′,4-tetrahydroxychalcone. Chalcone synthase has been purified from several species and antibodies to chalcone synthase have been produced. Chalcone synthase is reported to be expressed during different developmental stages and in response to various stress conditions (Cramer et al., EMBO J. 4:285-289 (1985); Koes et al., Plant Mol. Biol. 12:213-225 (1989)). Chalcone synthase clones have been reported from potato (Jeon et al., Biosci. Biotechnol. Biochem. 60:1907-1910 (1196)), rice (Reddy et al., Plant Mol. Biol. 32:735-743 (1996)), Pueraria lobata (Nakajima et al., Biol. Pharm. Bull 19:71-76 (1996)), Camellia sinensis (Takeuchi et al., Plant Cell Physiol. 35:1011-1018 (1994)), tomato (O'Neill et al., Mol. Gen. Genet. 224:279-288 (1990)), and buckwheat (Hrazdina et al., Arch. Biochem. Biophys. 247:414-419 (1986)).

Chaperones are proteins that bind to and stabilize other proteins to facilitate the correct folding of these proteins by mediating protein folding, unfolding, oligomerization, subcellular localization and proteolytic removal. Chaperones affect an array of cellular processes required for both normal cell function and survival of stress conditions (Boston et al., Plant Mol. Biol. 32:191-222 (1996)). Major classes of cytoplasmic chaperones include the heat shock proteins (HSPs) 100, 90, 80 and 70. Chaperones that are implicated in protein-protein interactions in the endoplasmic reticulum (ER) include glucose-regulated proteins (GRPs) 94 and 78, disulfide isomerase, the membrane-bound calcium-dependent protein calnexin (Boyce et al., Plant Phys. 106:1691 (1994)), and a calcium-binding protein calreticulin (Nelson et al., Plant Phys. 114:29-37 (1997)). Another class of chaperones comprise the cytosolic peptidyl-prolyl isomerases (cyclophilins).

Also involved in chaperone-mediated processes are immunoglobulin-binding proteins, thioredoxins, the Sec61 protein required for protein translocation across the ER membrane (Broughton et al., J. Cell Sci. 110:2715-2727 (1997)), and cytoplasmic proteasomes that degrade proteins from the ER. INA-treatment of Arabidopsis plants is reported to result in an upregulation of the expression of homologues of HSP90s, calreticulin, calnexin, cyclophilin, thioredoxins, Sec61, immunoglobulin-binding proteins and proteasomes.

Several proteins are reported to be upregulated upon pathogen infection, elicitor challenge or treatment with SAR-inducing chemicals, such as INA. These proteins are reported to play active roles in plant defense. One of the elicitor-inducible proteins is omega-6 fatty acid desaturase (FAD). Treatment of cultured parsley cells with a structurally defined peptide elicitor of fungal origin has been associated with changes in the levels of various desaturated fatty acids (Kirsch et al., Plant Phys. 115:283-289 (1997)).

Other elicitor and/or pathogen-inducible proteins include ahydrophilic regulatory protein with homology to 14-3-3 that regulates the plasma membrane H⁺ATPase (Jahn et al., Plant Cell 9:1805-1814 (1997)), tomato 1-aminocyclopropane-1-1-carboxylate synthase (ACC synthase) (Oetiker et al., Plant Mol. Biol. 34:275-286 (1997)), parsley chorismate mutase 1 (Sequence deposited with Genbank by O. Batz in 1997), licorice cytochrome P450 (CYP Ge-3) (Plant Phys. 115:1288 (1997)), pea disease resistance response protein 206-d (Culley et al., Plant Phys. 107:301-302 (1995)), pea disease resistance response protein DRRG49-c (Chiang et al., Mol. Plant Microbe Interact. 3:78-85 (1990)), rice GDP-dissociating inhibitor OsGDI1 (Sequence deposited with Genbank by C. Y. Kim in 1997), potato hydroxymethylglutaryl CoA reductase (Choi et al., Plant Cell 4:1333-1334 (1992)), parsley S-adenosylhomocysteine hydrolase (Kawalleck et al., Proc. Natl. Acad Sci. (U.S.A.) 89:471-34717 (1992)), tomato subtilisin-like endoprotease PR-P69 (Tornero et al., Proc. Natl. Acad. Sci. (U.S.A.) 93:6332-6337 (1996)), carrot ENOD8 homologous glycoproteins (Bertinetti et al., Mol. Plant Microbe Interact. 9:658-663 (1996)), and parsley tyrosine carboxylase (Kawalleck et al., J. Biol. Chem. 268:2189-2194 (1993)).

Additional proteins that are reported to be induced by jasmonic acid and/or salicylic acid (analogs) include wheat WCI1, wheat WCI5, and thiolprotease (Gorlach et al., Plant Cell 8:629-643 (1996)), Sauromatum alternative oxidase (Gorlach et al., Plant Mol. Biol. 21:615 (1993)), glucosyltransferase (Horvath et al., Plant Mol. Biol. 31:1061-1072(1996)), barley thionins (Andreson et al., Plant Mol. Biol. 19:193-204 (1992)), and pea disease resistance response protein PI206 (Plant Mol. Biol. 11:713-715 (1988)).

Another group of proteins with a function in the plant pathogen-response are the downstream components of the defense signaling pathway. This group of proteins can be divided into 5 classes. Members of the first class of defense genes are induced by a variety of biotic and abiotic agents, including salicylic acid, and encode the pathogenesis related (PR) proteins acidic PR1 (PR1a, PR1b, PR1c), acidic β-1,3-glucanase (PR2a, PR2b, PR2c), acidic class II chitinase (PR3a, PR3b, PRQ), hevein-like protein (PR4a, PR4b), thaumatin-like PR5, acidic- and basic isoforms of class III chitinase, extracellular β-1,3-glucanase, basic PR1, and SAR 8.2. In some cases, overexpression of individual PR genes has been reported to enhance disease control. For instance, overexpression of PR1a in tobacco was reported to result in increased resistance to the Oomycete pathogen Peronospora (Alexander et al., Proc. Natl. Acad. Sci. (U.S.A.) 90:7327-7331 (1993)). Overexpression of a tobacco osmotin-like PR5 in potato was reported to result in partial control of Phytophthora infestans (Liu et al., Proc. Natl. Acad. Sci. (U.S.A.) 91:1888-1892 (1994)).

A second class of defense genes induced by jasmonic acid encode for thionins (Andreson et al., Plant Mol. Biol. 19:193-204 (1992)). A third class of defense genes is also induced by jasmonic acid and encodes for protease inhibitors, some of which display activity against insects and nematodes.

A fourth class of defense genes are induced by pathogen infection and encode small basic lipid transfer proteins (LTPs), which are reported to participate in cutin biosynthesis, surface wax formation, adaptation of plants to environmental changes and pathogen-defense reactions (Kader, Trends Plant Sci., in press (1998)). A barley LTP applied on tobacco leaves eliminated symptoms caused by infiltration of Pseudomonas syringae. A fifth class of defense genes encodes for defensins or antifungal proteins.

7. Plant Proteases

Proteases are reported to be found within a number of plant cell compartments, including the nucleus, cytosol, golgi, mitochondria, chloroplast, and vacuole/protein body. In photosynthetic tissue, nearly 50% of total cell protein is localized in the chloroplast (Vierstra, Plant Mol. Biol. 32:275-302 (1996)). Although the bulk of plant proteases are located in the vacuole, inhibition of proteases located in the vacuole has not been reported to result in a rapid phytotoxic effect (Moriyasu, Plant Physiol. 109:1309-1315 (1995)). Proteases have been reported to play a role in apoptosis or programmed cell death (Duriez and Shah, Biochem. Cell Biol. 75:337-349 (1997)).

Proteolysis is reported to be essential for many aspects of plant physiology and development. For example, it has been reported to be responsible for cellular housekeeping and stress response by removing abnormal/misfolded proteins, for supplying amino acids for protein synthesis, for assisting in the maturation of zymogens and peptide hormones by selective cleavage, for controlling metabolism, homeosis, and development by reducing the abundance of key enzymes and regulatory proteins, and for programmed cell death of specific plant cells or organs.

The cytosol contains the 20S and 26S proteasome, and the ubiquitin proteolytic or ubiquitin conjugating pathway. The proteasome has been reported to be highly conserved, but components of the ubiquitin conjugating pathway have been reported to be diverse. The ubiquitin conjugation pathway involves an E2 enzyme and/or an E3 enzyme. Seventeen reported Arabidopsis U2 genes have been assigned to six different groups (Vierstra, Plant Mol. Biol. 32:275-302 (1996)).

Inhibition of the 20S and 26S proteasome has been reported to modify plant senescence processes (Devereaux et al., J. Biol. Chem. 270:29660-29663 (1995)). Proteolytic pathways have been engineered to remove specific proteins by modification of ubiquitin conjugating enzymes (E2s) (Gosink and Vierstra, Proc. Natl. Acad. Sci. (U.S.A.) 92:9117-9121 (1995)). A protein-binding domain specific to a target protein has been fused to the C-terminus of E2, thus facilitating ubiquitination and ATP-dependent degradation of the target protein. It has been reported that a plant can be “immunized” against pathogen attack by targeting key pathogen proteins for destruction (Vierstra, Plant Mol. Biol. 32:275-302 (1996)).

Cysteine and serine proteases have been reported to be induced during xylogenesis in Zinnia elegans (Ye and Varner, Plant Mol. Biol. 30:1233-1246 (1996)). During the process of xylogenesis, autolysis has been reported to be essential to the formation of a tubular system in the plant for conductance of water and solutes. A thermostable serine protease from melon fruit (Cucumis melo), cucumisin, has been reported (Yamagata et al., J. Biol. Chem. 269:32725-32731 (1994)).

SPARC (secreted protein acidic and rich in cysteine) is a conserved metal-binding extracellular matrix glycoprotein expressed during embryogenesis. It has been reported that SPARC plays a role in the regulation of cell adhesion and proliferation (Damjanovski et al., Dev. Genes Evol. 207:453-461 (1998); Gilmour et al., EMBO J. 17:1860-1870 (1998); Shiba et al., J. Cell. Physiol. 174:194-205 (1998)). SPARC has been reported to be a secreted Ca²⁺-binding glycoprotein (Gilmour et al., EMBO J. 17:1860-1870 (1998)).

Nth1, neutral trehalase, functions to make trehalose and has been reported to play a role in germination of spores (Nwaka et al., J. Biol. Chem. 270:10193-10198 (1995)). Nth1 has also been reported to be induced in response to heat or chemical stress (Zaehringer et al., FEBS Lett. 412:615-620 (1997)), as well as osmotic stress (Hounsa et al., Microbiology 144:671-680 (1998)).

α-Amylase is a catabolic enzyme that degrades starch. α-Amylase in barley aleurone cells is expressed under control of the plant hormones gibberellic acid and abscisic acid, along with aleurain, a thiol (Whittier et al., Nucleic Acids Res. 15:2515-2535 (1987)). A cDNA encoding alpha amylase has been reported from rice (Terashima et al., Appl. Microbiol. Biotechnol. 43:1050-1055 (1995); Huang et al., Plant Mol. Biol. 14:655-668 (1990)), and barley (Khursheed et al., J. Biol. Chem. 263:18953-18960 (1988)).

An ATP-dependent protease, La, has been reported in E. coli; for reviews see Tanaka, Tanpakushitsu Kakusan Koso 30:441-459 (1985); Goldber et al., Biochem. Soc. Trans. 15:809-811 (1987); Goldberg et al., Methods Enzymol. 244:350-375 (1994). La protease has been reported to be ubiquitous (Goldberg, Eur. J. Biochem. 203:9-23 (1992)). The complete nucleotide sequence of protease La from E. coli K12 has been reported (Amerik et al., Bioorg. Khim. 16:869-880 (1990)). One reported activity of La protease is its participation in the removal of aggregated proteins, such as those that result from heat shock (Laskowska et al., Mol. Microbiol. 22:555-571 (1996)). An E. coli protease La has been reported to have a DNA-binding site (Baker, FEBS Lett. 244:31-33 (1989)).

Saccharomyces cerevisiae Pim1 nuclear gene encodes a mitochondrial ATP-dependent protease that exhibits over 30% identity with ATP-dependent protease La, and has been reported to be required for mitochondrial function (Kutejova et al., FEBS Lett. 329:47-50 (1993)). Pim1 has also been reported to play a role in the heat shock response (Van Dyck et al., J. Biol. Chem. 269:238-242 (1994)).

Subtilisin-like processing proteases have been reported to be associated with pre-protein and pro-hormone processing (Komano and Fuller, Proc. Natl. Acad. Sci. (U.S.A.) 92:10752-10756 (1995), and have mammalian and plant homologues (Bathurst et al., Science 235:348-350 (1987); Brennan et al., J. Biol. Chem. 265:21494-21497 (1990); Hatsuzawa et al., J. Biol. Chem. 265:22075-22078 (1990); Thomas et al., J. Biol. Chem. 265:10821-10824 (1990); Vierstra, Plant Mol. Bio. 32:275-302 (1996)).

An aspartyl protease, Mkc7, along with yeast aspartic acid protease 3 (Yap3) are processing proteases located in the golgi apparatus of Saccharomyces cerevisiae (Komano and Fuller, Proc. Natl. Acad. Sci. (U.S.A.) 92:10752-10756 (1995); Zhang et al., Biochim. Biophys. Acta 1359:110-122 (1997)). Mkc7 has been reported to be a membrane associated protein. Aspartic acid YAP3 is an endoprotease that has been reported to cleave at paired basic residues (Azaryan et al., J. Biol. Chem. 268:11968-11975 (1993); Copley et al., Biochem. J. 330:1333-1340 (1998)). In yeast, Yap3 has been reported to be associated with the secretory pathway and the cleavage of a pro-alpha-mating factor (Ledgerwood et al., FEBS Lett. 383:67-71 (1996)).

Kex2 endoprotease of the yeast Saccharomyces cerevisiae has been reported to be a prototype of a family of eukaryotic subtilisin homologues (Gluschankof and Fuller, EMBO J. 13:22808 (1994)). Kex2 and Yap3 endoproteases have been reported to have distinct, but overlapping, substrate specificities (Bourbonnais et al., Biochimie 76:226-233 (1994)). Some subtilisin-like proteases, such as P69, are induced in plant hosts upon virus pathogen attack (Tornero et al., Proc. Natl. Acad. Sci. (U.S.A.) 93:6332-6337 (1996); Tornero et al., J. Biol. Chem. 272:14412-14419 (1997)). P69 has been reported to be a secreted calcium-activated endopeptidase.

Subtilases have also been reported to be involved in both symbiotic and nonsymbiotic processes in plant development. A subtilisin protease Ag12 has been reported to be associated with actinorhizal nodule development in root nodules of Alnus glutinos (Ribeiro et al., Plant Cell 7:785-794 (1995)). A homologue of Ag12, Ara12, has been reported in Arabidopsis. Ara12 has been reported to be expressed in all organs, and its expression levels were highest during silique development (Ribeiro et al., Plant Cell 7:785-794 (1995)). A virally encoded antifungal toxin in transgenic tobacco, KP6 killer toxin, has been reported to be processed by the subtilisin-like processing protease, Kex2p that is present in both fungal and plant cells (Kinal et al., Plant Cell 7:677-688 (1995)).

Plant homologues to E. coli FtsH protease, an ATP-dependent metalloprotease, have also been reported (Lindahl et al., J. Biol. Chem. 271:29329-29324 (1996)). FtsH protease has been reported to be involved in photosystem assembly (Ostersetzer and Adam, Plant Cell 9:957-965 (1997)).

Bacterial FtsH proteases have been reported to be membrane-bound, ATP-dependent zinc-metalloproteinases (Akiyama et al., Guidebook Mol. Chaperones. Protein-Folding Catal, Oxford University Press (1997); Akiyama et al., Mol. Microbiol. 28:803-812 (1998)). FtsH has been reported to act on a subset of unstable proteins, and function as a molecular chaperone ((Akiyama et al., Guidebook Mol. Chaperones. Protein-Folding Catal, Oxford University Press (1997); Suzuki et al., Trends Biochem. Sci. 22:118-123 (1997)). In bacteria, FtsH protease has been reported to participate in a secretory pathway (Ito et al., Membrane Proteins: Structure, Function, Expression Control, International Symposium (Basel, Switzerland) (1997)), and it has been reported to participate in at least two pathways for protein degradation (Kihara et al., J. Mol. Biol. 279:175-188 (1998)). In Bacillus subtilis FtsH is a general stress gene which has been reported to be transiently induced after thermal or osmotic upshift (Deuerling et al., Mol. Microbiol. 23:921-933 (1997)).

FtsH homologues have been reported in the plastids of higher plants both by immunological cross reactivity (Lindahl et al., J. Biol. Chem. 271:29329-29334 (1996); Ostersetzer et al., Plant Cell 9:957-965 (1997)), and DNA sequence homology (Lindahl et al., J. Biol. Chem. 271:29329-29334 (1996); Wolfe, Curr. Genet. 25:379-383 (1994)). A chloroplast FtsH has been reported to be associated with the degradation of improperly assembled Rieske FeS protein (RISP) imported into in vitro chloroplasts (Ostersetzer et al., Plant Cell 9:957-965 (1997)). FtsH has also been reported to have chaperone-like activity (Akiyama et al., Guidebook Mol. Chaperones. Protein-Folding Catal, Oxford University Press (1997)). FtsH has been reported to participate in the assembly of protein into and through the membrane (Akiyama et al., J. Biol. Chem. 269:5218-5224 (1994)). An Arabidopsis cDNA encoding FtsH has been reported (Lindahl et al., J. Biol. Chem. 271:29329-29334 (1996)).

The D1 protein of the photosystem II (PSII) complex in the thylakoid membrane of oxygenic photosynthetic organisms has been reported to be synthesized as a precursor polypeptide (pD1) with a C-terminal extension (Anbudurai et al., Proc. Natl. Acad. Sci. (U.S.A.), 91:8082-8086 (1994)). Post-translational processing of the pD1 protein has been reported to be essential to establish water oxidation activity of the PSII complex. CtpA (photosystem II Dl protease) is a carboxyl terminal processing protease that cleaves a carboxyl terminal 9 residue peptide (in higher plants) from pre-DI protein, forming the active D1 (Anbudurai et al., Proc. Natl. Acad. Sci. (U.S.A.), 91:8082-8086 (1994); Bowyer et al., J. Biol. Chem. 267:5424-5433 (1992); Taylor et al., FEBS Lett. 237:229-233 (1988)). Active CtpA protease has been reported to be required for photosynthetic activity in both the blue green algae Synechocystis sp. (Shestakov et al., J. Biol. Chem. 269:19354-19359 (1994)), as well as the green algae Scenedesmus obliquus (Diner et al., J. Biol. Chem. 263:8972-8980 (1988); Taylor et al., FEBS Lett. 237:229-233 (1988)). Failure to correctly process the pre-DI protein has been reported to result in a non-functional manganese cluster responsible for photosynthetic water oxidation (Trost et al., J. Biol. Chem. 272:20348-20356 (1997)).

Homologues to CtpA have been reported in Bartonella bacilliformis (Mitchell and Minnick, Microbiology 143:1221-1233 (1997)), and E. coli (Tsp protease) (Silber et al., Proc. Natl. Acad. Sci. (U.S.A.) 89:295-299 (1992)). A full-length CtpA cDNA from barley (Pakrasi et al., Photosynth.: Light Biosphere, Proc. Intl. Photosynth. Congr., 10^{th} (Dordrecht, Netherlands) (1995); EMBL accession No. X90558) and from spinach have been reported (Inagaki et al., Photosynt.: Light Biosphere, Proc. Intl. Photosynth. Congr., 10^{th} (Dordrecht, Netherlands); Inagaki et al., Plant Mol. Biol. 30:39-50 (1996)). It has been reported that inhibition of CtpA activity results in phytotoxicity. It has been reported that D1 protease has been cloned and sequenced from wheat, Scenedesmjus obliquus and Synechocystis.

Processing proteases other than CtpA, such as leader peptidases have been reported in plants (Barbook et al., FEBS Lett. 398:198-200 (1996)). In addition to processing protease CtpA, there has been another reported proteolytic mechanism for removing photo-damaged D1 protein from photosystem II reaction centers. Chloroplast homologues to E. coli ATP-dependent Clp protease and cyanobacterial Ca²⁺-stimulated protease have been reported (Ostersetzer et al., Eur. J. Biochem. 236:932-936 (1996)).

ClpP has been reported to be a protease subunit of a two-component, ATP-dependent protease reported in E. coli (Katayama et al., J. Biol. Chem. 263:15226-15236 (1988)). The regulatory subunit has been reported to be ClpA, and the complex has been reported to require Mg²⁺ and ATP for activity (Katayama et al., J. Biol. Chem. 263:15226-15236 (1988)). It has also been reported that ClpA exhibits chaperonin-like activity (Kessel et al., J. Mol. Biol. 250:587-594 (1995); Suzuki et al., Trends Biochem. Sci. 22:18-123 (1997)). ATP hydrolysis by ClpA has been reported as being required for both the assembly and dissociation of the ClpA/P complex (Chung et al., Biol. Chem. 377:549-554 (1996)). In bacteria, a ClpA/P complex has been reported to be associated with a number of cellular processes including degradation of carbon starvation proteins (Damerau and St. John, J. Bacteriol. 175:53-63 (1993)), regulation of the sigma starvation factor (σ-s) (Schweder et al., J. Bacteriol. 178:470-476 (1996)), removal of heat shock damaged proteins (Laskowska et al., Mol. Microbiol. 22:555-571 (1996)), and degradation of proteins arising from truncated open reading frames (Herman et al., Genes Dev. 12:1348-1355 (1998)). It has been reported that the function of ClpP protease is similar to that of the eukaryotic 20S proteosome. It has been reported that ClpP component of E. coli enzyme is a structural homologue of the 20S proteosome, consisting of two heptamers, stacked on top of each other in a head-to-head fashion to form a tetradecamer (Shin et al., Proc. Natl. Acad. Sci. (U.S.A.) 262:71-76 (1996)).

Homologues of bacterial ClpA/P genes have been reported in both algae and plastids of higher plants (Desimone et al., Bot. Acta 110:234-239 (1997); Gray et al., Plant Mol. Biol. 15:947-950 (1990); Weiss-Wichert et al., Photosynthesis: Light Biosphere, Proc. Int. Photosynth. Cong., 10^{th} (Dordrecht, Netherlands) (1995); Berges and Freeman, J. Phycol. 32:566-574 (1996); Ostersetzer et al., Eur. J. Biochem. 236:932-936 (1996)). A clpA plastid homologue has also been termed ‘clpC’. A clpP gene in the green algae Chlamydomonas reinhardtii gene has been reported to be essential for cell growth (Huang et al., Mol. Gen. Genet. 244:151-159 (1994)). The function of plastid ClpA/P has been reported to be similar to the bacterial complex. ClpA has been reported to be induced both by water stress and during senescence in Arabidopsis thaliana (Nakashima et al., Plant J. 12:851-861 (1997)). A cDNA, ERD1, isolated from one-hour dehydrated Arabidopsis thaliana plants has been reported to be homologous to ClpA (Nakashima et al., Plant J. 12:851-861 (1997)). In barley leafs, transcript levels of clpP have been reported to be higher in photosynthetically active leaves but decrease during senescence (Humbreck and Krupinska, J. Photochem. Photobiol. 36:321-326 (1996); Ostersetzer et al., Eur. J. Biochem. 236:932-936 (1996)). In pea seedlings, levels of the regulatory subunit clpC have been reported to be regulated by both light intensity and temperature (Ostersetzer and Adam, Plant Mol. Biol. 31:673-676 (1996)).

Lysosomal cysteine proteinases are proteolytic enzymes that in the mature form are localized in lysosomes and the catalytic activity of which is based on a cysteine residue in the active site (Runeberg-Roos et al., Eur. J. Biochem. 202:1021-1027 (1991)). Aleurain is a reported barley thiol protease closely related to mammalian cathepsin H (cathepsins B and H are involved in the processing of precursor proteins). Aleurone thiol protease mRNA has been reported to be regulated by the plant hormones gibberellic acid and abscisic acid. Aleurone thiol protease mRNA has also been reported to be expressed at high levels in leaf and root tissue. Aleurone thiol protease has been reported to represent the equivalent of a plant lysosomal thiol protease (Rogers et al., Proc. Natl. Acad. Sci. (U.S.A.), 82:6512-6516 (1985)). Barley aleurone layers have been reported to synthesize and secrete several proteases in response to gibberellic acid (GA3) (Whittier et al., Nucleic Acids Res. 15:2515-2535 (1987); Koehler and Ho, Plant Cell 2:769-783 (1990)).

Pseudotzain from Pseudotsuga menziesii (Douglas fir) is a protease reported to be involved in storage protein mobilization (g2118132; Tranbarger and Misra, Gene 172:221-226 (1996)). REP-1 has been reported to digest in vitro both the acidic and basic subunits of rice glutelin, the major seed storage protein of rice (g1514952; Kato and Minamikawa, Eur. J. Biochem. 239:310-316 (1996)). A vacuolar processing enzyme has been reported in soybean protein bodies that converts proproteins to the corresponding mature forms (g511937; Shimada et al., Plant Cell Physiol. 35:713-718 (1994)). CCP1 and CCP2 are two reported cDNA clones encoding maize seed cysteine proteinases. CCP1 has been reported to be homologous to pea 15a CP and Arabidopsis thaliana RD 19 CP, both of which have been reported to be induced in response to dehydration of the plant. CCP1 has been reported to be expressed in ripened maize seeds. CCP2 protease mRNA from maize seed is expressed only during germination, with maximum expression at the 3-day stage (g1688044; Domoto et al., Biochim. Biophys. Acta 1263:241-244 (1995)).

Granzymes are neutral serine proteases that are stored in specialized lytic granules of cytotoxic lymphocytes (Greenberg, Cell Death Differ. 3:269-274 (1996)). Granzyme B has been reported to play a role in lymphocyte-mediated target cell apoptosis (Smyth et al., J. Leukocyte Biol. 60:555-562 (1996); Pham and Ley, Semin. Immunol. 9:127-133 (1997)). It has been reported that granzymes have features that are strongly conserved, including consensus sequences at their N-termini and around 3 catalytic residues, activation from a zymogenic form, and conserved disulfide bridges (Smyth et al., J. Leukocyte Biol. 60:555-562 (1996)). A family of cysteine proteases homologous to the Caenorhabditis elegans cell death protein CED-3 has been reported to play an effector role in the process of apoptosis in mammals (Kumar and Lavin, Cell death Differ. 3:255-267 (1996); Kumar, Int. J. Biochem. Cell Biol. 29:393-396 (1997)). APAF-1 (apoptotic protease activating factor 1) participates in a caspase activation cascade triggered by cytochrome C release from mitochondria, leading to cell death (Asoh and Ohta, Shinkei Seishin Yakuri 19:967-970 (1997); Zou et al., Cell 90:405-413 (1997)).

Proteases have been reported to play a role apoptosis; late stages are connected with the activation of a cascade of intracellular proteases, which leads to massive protein destruction (Sukharev et al., Cell Death Differ. 4:457-462 (1997)). In mammals, two general protease classes involved in programmed cell death have been reported; serine proteases (granzymes) and cysteine proteases (caspases) (Sukharev et al., Cell Death Differ. 4:457-462 (1997)). Caspase family genes encode proenzyme forms that require proteolytic cleavage for activation. Cell death signaling has been reported to involve mutual activation of several proteases, which in turn cleave several structural and catalytic proteins, resulting in the cleavage of proteins involved in the cellular repair system.

Leaf senescence has been studied under both natural conditions and in a model system. Under natural conditions, flag leaves of field-grown barley plants have been characterized according to different parameters indicating the onset and course of senescence (Humbeck and Krupinska, J. Photochem. Photobiol. 36:321-326 (1996)). Under the model system, senescence has been studied with barley primary foliage leaves which are induced to senescence by transfer of young plants to darkness (Humbeck and Krupinska, J. Photochem. Photobiol. 36:321-326 (1996)). It has been reported that senescence in plants is a developmental process that falls into two major senescent mechanisms, nutrient deficiencies and genetic programming (Nooden et al., Physiol. Plant 101:746-753 (1997)).

In plants, one of the reported cell death model systems is the differentiation of Zinnia elegans mesophyll cells into tracheary elements (TEs). During this process a transient and specific expression of a cysteine endopeptidase activity similar to papain associated with developmentally programmed cell death has been reported (Minami and Fukuda, Plant Cell Physiol. 36:1599-1606 (1996); Ye and Varner, Plant Mol. Biol. 30:1233-1246 (1996)). Three proteinases have been reported to be exclusive to differentiating TEs, and a fourth proteinase has been reported to be most active in differentiating TEs (Beers and Freeman, Plant Physiol. 113:873-880 (1997)). In barley, nucellar cells undergo programmed cell death after ovule fertilization. A gene that encodes an aspartic protease-like protein termed ‘nucellin’ has been reported to be expressed in nucellar cells during their degeneration (Chen and Foolad, Plant Mol. Biol. 35:821-831 (1997)).

In pharmaceutical drug discovery proteases have been reported to be attractive targets for the discovery of small molecule inhibitors. Human proteases including matrix metalloproteinases and thrombin, and the viral processing proteases including HIV-1 protease and assemblin, have all been targets of chemical discovery efforts (see, e.g., Hilpert et al., J. Med. Chem. 37:3889-3901 (1994); Weston and Sindelar, Curr. Med. Chem. 3:37-46 (1996); Whittle and Blundell, Annu. Rev. Biophys. Biomol. Struct. 23:349-375 (1994)). Proteases or components of protease pathways have application toward crop improvement, such as modification of protein content, and pathogen defense (see, e.g., Hondred and Vierstra, Curr. Opin. Biotechnol. 3:147-151 (1992)).

8. Protein Kinases

Protein kinases are enzymes that transfer a phosphate group from a phosphate donor onto an acceptor protein. Based on the amino acid specificity, protein kinases can be grouped into at least five categories (Hunter, Methods Enzymol. 200:3-37 (1991)) protein-serine/threonine kinases that phosphorylate serine or threonine on target proteins; 2) protein-tyrosine kinases that transfer phosphate to tyrosine of target proteins; 3) protein-histidine kinases that phosphorylate histidine, arginine, or lysine of target proteins; 4) protein-cysteine kinases that phosphorylate cysteine of target proteins and 5) protein-aspartyl or glutamyl kinases that are phosphotransferases with a protein acyl group as acceptor. As the number of reported protein-kinases increases, such classification has been found to be problematic. For example, some protein kinases have been found to have the ability to phosphorylate both serine/threonine and tyrosine on target proteins (Featherstone and Russell, Nature 349:808-811 (1991); Stem et al., Mol. Cell. Biol. 11:987-1001 (1991); Feng et al., Biochem. Biophys. Acta 1172:200-204 (1993)).

Protein kinases have catalytic and regulatory domains or subunits. In most single subunit protein kinases, the catalytic domain usually lies near the carboxyl terminus, stretching from 250 to 300 amino acids in length, while the amino terminus is devoted to a regulatory role. In protein kinases having a multiple subunit structure, subunits consisting almost entirely of catalytic domain are common. Hanks and Quinn, (Methods Enzymol. 200:38-63 (1991)) have reported that there are 11 conserved regions referred to as subdomains, some of which contain invariant or near invariant residues. Eukaryotic serine/thronine and tyrosine protein kinases have been reported, from phylogenetic analysis, to fall into one of five supergroups: (a) an “AGC” group, consisting of cyclic nucleotide-dependent protein kinase A (PKA), protein kinase G (PKG), calcium-phospholipid-dependent protein kinase C (PKC) and ribosomal S6 kinase families; (b) a CaMK” group, consisting of calcium-/calmodulin-dependent kinases and SNF1/AMP-activated protein kinase families; (c) a “CMGC” group, consisting of cyclin-dependent kinase (CDP), mitogen-activated protein kinase (MAPK), glycogen synthetase kinase (GSK-3), and casein kinase II (CKII) families; (d) a protein tyrosine kinase (PTK) group and (e) an other group which contains protein kinases having no clear structural similarity to any of the above groups (Hanks and Hunter, In: Protein Kinase Factsbook, pp. 7-47, Hardie and Hanks eds, Academic Press, London (1995)).

Protein kinases play roles in the regulation of protein and enzyme activity in the transduction of environmental, developmental, and metabolic signals in animals and simple eukaryotes. It has been reported that protein kinases also act as signal transducers in plants. Activities of plant protein kinases have been reported to be responsive to various environmental stimuli and developmental changes (see reviews by Ranjeva and Boudet, Ann. Rev. Plant Physiol. 38:73-93 (1987); Roberts and Harmon, Annu. Rev. Plant Physiol. Plant Mol. Bio. 43:37-414 (1992); Huber et al., Int. Rev. Cytol. 149:47-98 (1994)). Although most of reported plant protein kinases serine/threonine-protein kinases (Stone and Walker, Plant Physiol. 108:451-457 (1995)) other types of protein kinases have been reported in plants. For example, histidine protein kinase-like sequence has been reported in plant phytochrome (Schneider-Poetsch, Photochem. Photobiol. 56:839-846 (1992)). The Arabidopsis ETR1 gene has been reported to encode a histidine-kinase ethylene receptor (Chang et al., Science 262:539-568 (1993)). Activities of tyrosine-specific protein kinases also have been found in plants (Torruella et al., J. Biol. Chem. 261:6651-6653 (1986); Trojanek et al., Eur. J. Biochem. 235:338-344 (1996)).

PVPK-1 (Lawton et al., Proc. Natl. Acad. Sci. (USA) 86:3140-3144 (1989)) and APTKs (Zhang et al., J. Biol. Chem. 269:17586-17592 (1994)) are plant protein kinases which have been reported to belong to the AGC group. PVPK-1 is a member of a group of protein kinases that have putative catalytic domains most closely related to PKA and PKC. PVPK-1 exhibits no homology to regulatory domains of PKA and PKC or other protein kinases. APTK1 has been reported to be expressed in all tissues and all developmental stages, with the greatest expression in metabolic active tissues. Arabidopsis aptk genes have been reported to encode protein kinases with sequence similarity in the catalytic domain to animal S6 protein kinase, PKA and PKC (Hayashida et al., Gene 124:251-255 (1993); Zhang et al., Biol. Chem. 269:17586-17592 (1994).

In yeast, SNF1 protein kinases regulate carbon catabolite repression/depression (Gancedo, Eur. J. Biochem. 206:297-313 (1992)). SNF1 protein kinases have been reported to play a major role in the control of lipid metabolism in mammals (Hardie, Biochem. Biophys. Acta 1123:231-238 (1992); Hardie et al., Trends Biochem. Sci. 14:20-23 (1989)). SNF1 protein kinases have a N-terminal catalytic domain and a C-terminal region that interacts with other proteins. A SNF1-like clone, cRKIN1, has been reported from a rye endosperm cDNA library (Alderson et al., Proc. Natl. Acad. Sci. (USA) 88:8602-8605 (1991)). Wheat WPK4 is another member of the SNF1 kinase family. WPK4 has been reported to have increased transcript levels in response to multiple stimuli such as light, nutrient deprivation, and cytokinin application (Sano and Youssefian, Proc. Natl. Acad. Sci. (USA) 91:2582-2586 (1994)). Many SNF1-like genomic and cDNA clones have been isolated from Arabidopsis (Le Guen et al., Gene 120:249-254 (1992)); barley (Halford et al., Plant. J. 2:87-96 (1992)); tobacco (Muranaka et al., Mol. Cell. Biol. 14:2958-2965 (1994)) and Mesembryanthemum crystallinum L. (Baur et al., Plant Physiol. 106:1225-1226 (1994)).

In mammalian cells, AMP-activated protein kinase is activated allosterically by 5′-AMP. AMP-activated protein kinases play an important role in the regulation of lipid metabolism by phosphorylating acetyl-CoA carboxylase. Acetyl-CoA carboxylase catalyses the first committing step in fatty acid synthesis. AMP-activated protein kinase also phosphorylates 3-hydroxy-3-methylglutaryl-CoA reductase (HMG-CoA reductase), a regulatory enzyme in isoprenoid biosynthesis. Activity of HMG-CoA reductase kinase (also known as HRK-A) (Ball et al., Eur. J. Biochem, 219:743-750 (1994)) has been reported from a number of plant extracts.

Plant possesses very unique calcium-dependent but calmodulin-independent protein kinase families (CDPKs). A soybean CDPK-α was the first CDPK gene isolated (Harper et al., Science 252:951-954 (1991); Roberts and Harmon, Annu. Rev. Plant Physiol. Plant Mol. Bio. 43:375-414 (1992)). The N-terminal region of CDPK-α exhibits homology with the protein kinase catalytic domain of the CaMK family, the central part has an autoinhibitory junction domain, and the C-terminal region has homology to calmodulin with EF-hand, calcium binding sites. CDPKs have been reported to be present in many plant species and are encoded by multigene families. CDPKs are associated with many physiological events that respond to transient changes in intracellular calcium levels (Roberts and Harmon, Annu. Rev. Plant Physiol. Plant Mol. Bio. 43:375-414 (1992); Gilroy and Trewavas, Bioassays 16:677-682 (1994)). An example of CDPK's role in the regulation of plant physiological events is that the opening of an iron channel is modulated by the phosphorylation of soybean nodulin 26 by CDPK (Lee et al., J. Biol. Chem. 270:27051-27057 (1995)). CDPKs are important components in stress signal transduction in maize protoplasts (Sheen, Science 274:1900-1902 (1996)). CDPKs also have been reported to phosphorylate sucrose synthetase, an important carbon metabolic enzyme in many sink tissues. Phosphorylation of the sucrose synthetase changes the kinetics of sucrose synthetase (Huber et al., Plant Physiol. 112:793-802 (1996)).

Processes through the eukaryotic cell cycle have been reported to be regulated by cyclin-dependent protein kinase, along with its regulatory subunit, cyclin. In yeast, a single CDK gene (cdc2 in Schizosaccharomyces pombe and cdc28 in Saccharomyces cerevisiae) is required for cell cycle transition (Norbury and Nurse, Rev. Biochem. 61:441-470 (1992); Nasmyth, Curr. Opin. Cell Biol. 5:166-179 (1993)). In human cells, a family of CDK1 to CDK8 kinases have been reported to control the cell cycle (Pines, Biochem. Soc. Trans. 24:15-33 (1996)). CDKs have been reported to have a conserved PSTAIRE motif in subdomain III of the catalytic domain. Plant cells are similar to animal cells in that they contain multiple cyclin-dependent kinases. An Arabidopsis gene, AFC1, has been reported to complement a yeast mutant defective in the STE12-dependent signal transduction pathway (Bender and Fink, Proc. Natl. Acad. Sci. (USA) 91:12105-12109 (1994)). Highly homologous cdc2 genes have been reported from several other plant species, including maize, rice, alfalfa, soybean, Antirrhinum, and pea (Colasanti et al., Proc. Natl. Acad. Sci. (USA) 88:337-3381 (1991); Hashimoto et al., Mol. Gen. Genet. 233:10-16 (1992); Hirt et al., Plant J. 4:61-69 (1993); Miao et al., Proc. Natl. Acad. Sci. (USA) 90:943-947 (1993); Fobert et al., EMBO J. 13:616-624 (1994); Jacobs, Annu. Rev. Physiol. Mol. Biol. 46:317-339 (1995); Magyar et al., Plant Cell 9:223-235 (1997)).

Mitogen-activated protein kinase (MAPK) (also known as extracellular signal regulated kinase or ERK) signaling cascade is one of the pathways by which extracellular stimuli are transduced in intracellular responses. Activation of MAPKs requires both tyrosine and threonine phosphorylation by a dual specific MAP kinase kinase that in turn has to be activated by a serine/threonine MAP kinase kinase kinase (Posada and Cooper, Science 255:212-215 (1992); Marshall, Curr. Opin. Genet. Dev. 4:82-89 (1994)). The highly conserved threonine and tyrosine residues are located close to MAP kinase domain VIII. The MAPK family has been reported from a diverse array of organisms, including mammals, Xenopus, Drosophila, yeast, Dictyostelium, and plants (Durr et al., Plant Cell 5:87-96 (1993); Nishihama et al., Plant Cell. Physiol. 36:749-757 (1995); Raz and Fluhr, Plant Cell 5:523-530 (1995)). Polymerase chain reaction (PCR)-based homology clones from a variety of MAPK genes have been reported from several plant species, including MMK1 (also known as MsERK1 and Msk7), MMK2, MMK3, and MMK4 from alfalfa (Durr et al., Plant Cell 5:87-96 (1993); Jonak et al., Plant J. 3:611-617 (1993); Jonak et al., Mol. Gen. Genet. 248:686-694 (1995); Jonak et al., Proc. Natl. Acad. Sci. (USA) 93:11274-11279 (1996)), atMPK1 to atMPK7 from Arabidopsis (Mizoguchi et al., Plant. Mol. Biol. 21:279-289 (1993); Mizoguchi et al., Plant. J. 5:111-122 (1994); Mizoguchi et al., Proc. Natl. Acad. Sci. (USA) 93:765-769 (1996)), D5 (also known as PsMAPK) from pea (Stafstrom et al., Plant Mol. Biol. 22:83-90 (1993); Popping et al., Plant. Mol. Biol. 31:355-363 (1996)), Aspk9 (also known as AsMAP1) from oat (Huttly and Phillips, Plant Mol. Biol. 27:1043-1052 (1995)), NPK1 and NPK2 from tobacco (Banno et al., Mol. Cell. Biol., 13:4745-4752 (1993); Shibata et al., Mol. Gen. Genet. 246:401-410 (1995)) and PMEK1 from petunia (Decroocq-Ferrant et al, Plant Mol. Biol. 27:339-350 (1995)). MAPKs are involved in a variety of signaling processes in plants such as cell proliferation (Jonak et al., Plant. J. 3:611-617 (1993); Mizoguchi et al., Plant Mol. Biol. 21:279-289 (1994)), cold, drought, dehydration, salinity stress responses (Jonak et al., Proc. Natl. Acad. Sci. (USA) 93:11274-11279 (1996); Mizoguchi et al., Proc. Natl. Acad. Sci. (USA) 93:765-769 (1996)), and pathogen eliciting (Suzuki and Shinshi, Plant Cell 7:639-647 (1995); Mizoguchi et al., Trends Biotechnol. 15:15-19 (1997)).

In mammals, glycogen synthetase kinase-3 (GSK-3) has been reported to phosphorylate glycogen synthetase (Woodgett and Cohen, Biochem. Biophys. Acta 788:339-347 (1984)) and transcription factors such as c-jun, and c-myb (Boyle et al., Cell 64:573-584 (1991)) and L-myc (Saksela et al., Oncogen 7:347-353 (1992)). GSK-3 is identical to factor A, the activator protein phosphotase-1 (Hughes et al., EMBO J. 12:803-808 (1993)) and has been reported to show functional homology to a Drosophila gene, shaggy/zeste-white 3 which is required for several developmental processes in the embryo, larvae and adult. In the budding yeast, Saccharomyces cerevisiae, meiosis and expression of early meiotic genes are dependent upon Rim11p, a protein kinase related to GSK-3 (Malathi et al., Mol. Cell. Biol. 17:7230-7236 (1997)). GSK-3 also has been reported to be important for dorsoventral patterning in Xenopus embryos (He et al., Nature 374:617-622 (1995)). Plant members of GSK-3 family have been reported to be encoded by small multigene families. Arabidopsis clones of at least five GSK-3 homologous genes, asks, have been reported (Bianchi et al., Mol. Gen. Genet. 242:337-345 (1994)). Three msks clones from alfalfa have been reported to have 65-70% identity to GSK-3 (Pay et al., Plant J. 3:847-856 (1993)). ASKs and MSKs have both been reported to perform similar functions as mammalian GSK-3.

Casein kinase II (CKII) is a multifunctional protein kinase that plays a role in the control of cellular functions such as cell division and growth, gene expression, and DNA replication in all eukaryotes (Meisner and Czech, Curr. Opin. Cell. Biol. 3:474-483 (1991); Kikkawa et al., Mol. Cell. Biol. 12:5711-5723 (1992)). CKII has been reported to be a predominantly nuclear enzyme (Krek et al., J. Cell Biol. 116:43-55 (1992)). CKII isolated from animals and yeast possess a conserved characteristic tetrameric structure, α₂β₂, composed of two catalytic α subunits and two β regulatory subunits (Tuazon and Traugh, Adv. Sec. Mess. Phosphoprotein Res. 23:123-164 (1991)). In contrast, plant CKII has been reported in two different forms: a monomeric form and an oligomeric form whose subunit composition has not been investigated (Dobrowolska et al., Biochem. Biophys. Acta 1129:139-140 (1991); Dobrowolska et al., Eur. J. Biochem. 204:299-303 (1992); Li and Roux, Plant. Physiol. 99:686-692 (1992)). CKII clones from plants for both the catalytic protein kinase a and the regulatory β subunits have been reported (Dobrowoska et al., Biochem. Biophys. Acta 1129:139-140 (1991); Mizoguchi et al., Plant. Mol. Biol. 21:279-289 (1993); Colinge and Walker, Plant Mol. Biol. 25:629-658 (1994). Arabidopsis CKII has been reported to phosphorylate and promote the DNA-binding activity of a transcription factor that binds to the G-box promoter element found in various plant promoters (Klimczak et al., Plant Cell 7:105-115 (1995)). CKII has been reported to be associated with the phosphorylation of chloroplast photosystem II subunit (Testi et al., FEBS Letters 399:245-250 (1996)).

Many reported plant protein kinases do not fall into classical kinase families. For example, TSL from Arabidopsis is a serine/threonine kinase with limited similarity to other kinases (Roe et al. Cell 75:939-950 (1993)). TSL has been reported to be required in the floral meristem for correct initiation of the floral organ primordia and for proper development of organ primordia. Arabidopsis CTR1, cloned by insertional mutagenesis, is a putative serine/threonine protein kinase that has been reported to be similar to the Raf protein kinase found in mammals (Kieber et al., Cell 72:427-441 (1993)) and is a negative regulator of ethylene signal transduction.

Receptor-like protein kinases (RLKS) represent a group of unique plant protein kinases. RLKs are composed of an extracellular domain that functions in ligand binding, a transmembrane domain, and a localized serine/threonine protein kinase domain which is responsible for transducing signals. RLKs are structurally similar to animal growth factor receptor protein kinases. Based on the structural similarities of the extracellular domains the RLKs fall into three categories: the S-domain class, the leucine-rich-repeat class and a third class with epidermal-growth-factor-like repeats (Walker, Plant Mol. Biol. 26:1599-1609 (1994)). An additional type of RLK homologous proteins has been reported with the extracellular region related to plant defense proteins (Wang et al., Proc. Natl. Acad. Sci. (USA) 93:2598-2602 (1996)).

ZmPK1 was a first putative RLK isolated from the root of maize (Walker and Zhang, Nature 345:743-746 (1990)). The predicted extracellular domain of ZmPK1 has been reported to show homology with S-locus glycoprotein from Brassica and also has been reported to be involved in self-incompatibility (Stein et al., Proc. Natl. Acad. Sci. (USA) 88:8816-8820 (1991); Walker, Plant Mol. Biol. 26:1599-1609 (1994); Kumar and Trick, Plant J. 6:807-813 (1994)). RLKs also have been reported to play functional roles in disease resistance and plant development (Song et al., Science 270:1804-1806 (1995); Torri et al., Plant Cell 8:735-746 (1996); Becraft et al., Science 273:1406-1409 (1996); Lee et al., J. Biol. Chem. 270:27051-27057 (1996)).

Pto is another unique plant serine/threonine protein kinase. Pto plays roles in signaling and membrane targeting, and functions as an elicitor receptor (Martin et al., Science 262:1432-1436 (1993); Martin et al., Plant Cell 6:1432-1436 (1994); Tang et al., Science 274:2060-2063 (1996)). Pto has been reported to interact, directly or indirectly, with another protein in the Pto pathway, Pseudomonas resistance and fenthion sensitivity (Prf) protein which contains a leucine rich repeat (Salmeron et al., Cell 86:123-133 (1996); Salmeron et al., Plant Cell 6:511-520 (1994)). Pto has been reported to interact with a second serine/threonine protein kinase, Pti1, which is located downstream in the signal transduction pathway for bacterial speck-resistance in tomato and has been implicated in the pathway leading to the hypersensitive response and cell death (Zhou et al., Cell 83:925-935 (1995)). Pto also has been reported to interact with Pti4, Pti5, and Pti6. Pti4, Pti5, and Pti6 have been reported to be associated with the transcriptional activation of genes encoding defense proteins, called pathogenesis related genes, which play a role in establishing systemic acquired resistance (Zhou et al., EMBO J. 16:3207-3218 (1997)).

9. Antifungal Proteins

Plants protect themselves from fungal and microbial attack via several metabolic mechanisms. These include physiological changes that cause the plant surface to become impenetrable, the biosynthesis of enzymes that convert substrates in the plant to small organic molecules that are toxic to microbial invaders and the expression of proteins that have direct antimicrobial activity.

It has been reported that upon pathogen attack and other stresses, plants will alter their metabolism to express genes that enable them to cope with the new hostile environment. These genes are termed pathogenesis-related (PR) genes. PR genes have been characterized from several plant-pathogen systems and have been found in the majority of cases to encode proteins that have direct antifungal activity (Bowles, Annu. Rev. Biochem. 59:873-907 (1990), Bol et al., Annu. Rev. Phytopathol. 28:113-138 (1990)).

In contrast, plants have genes that constitutively express active antifungal proteins in various tissues. Most of the reported antifungal proteins have even numbers of cysteine residues, are small (less than 10 kDa for the monomeric unit) and basic (Broekaert et al., Crit. Rev. in Plant Sci. 16:297-323 (1997)).

PR1 protein was first reported in tobacco mosaic virus infected tobacco (Van Loon and Van Kammen, Virology 40:199-201 (1970)). This class of protein is represented by both basic and acidic members of approximately 14 to 15 kDa. Three reported versions of PR1 have in vitro activity against the fungal pathogen of tomato, Phytophthora infestans (Niderman et al., Plant Physiol. 108:17-27 (1995)).

β-1,3-glucanases (EC 3.2.1.39) are hydrolytic enzymes that digest the substrate β-1,3-glucan to glucose residues. β-1,3-glucanases are effective against plant pathogenic fungi of the Oomycete family as the cell walls of Oomycete members are composed of primarily β-1,3-glucans. Acidic and basic forms of β-1,3-glucanases have been reported as pathogenesis-related proteins in several plants including potato (Kombrink et al., Proc. Natl. Acad. Sci. (USA) 85:782-786 (1988)) and tomato (Joosten and de Wit, Plant Physiol. 89:945-951 (1989)). β-1,3-glucanases have been reported to be approximately 31 to 35 kDa. β-1,3-glucanases have also been reported to exert direct antifungal activity by inhibiting hyphal growth (Mauch et al., Plant Physiol. 88:936-942 (1988)).

Chitinases have been found in several plants as pathogenesis-related proteins that have direct antifungal activity (Schlumbaum et al., Nature 324:365-367 (1986)). Chitinases are hydrolases (EC 3.2.1.14) that can digest chitin, (also known as β-1,4-N-acetylglucosamine), a constituent of the cell wall of many plant pathogenic fungi. Chitinases have been reported to range from 27 to 34 kDa and exist as both basic and acidic isoforms (Kombrink et al., Proc. Natl. Acad. Sci. USA 85:782-786 (1988)).

Osmotin/thaumatin-like class of pathogenesis-related proteins have been reported in a variety of plants. Osmotin/thaumatin-like proteins have been reported as existing as both basic and acidic isoforms ranging from 22 to 26 kDa which exert direct antifungal activity (Vigers et al., Mol. Plant-Microbe Interact. 4:315-323 (1991)). Promoters that control expression of certain gene family members of this class have been reported to be wound-inducible (Zhu et al., Plant Physiol. 108:929-937 (1995)).

Plant defensins are 45 to 54 amino acids in length and possess eight disulfide linked cysteines. The disulfide linkage pattern has been reported to be X₃CX₁₀CX₅CX₃CX₁₀CX₈CXCX₃C, wherein the first cysteine residue (amino acid residue 4) is linked to the last cysteine residue (amino acid position 51, the second cysteine residue (amino acid residue 15) is linked to fifth cysteine residue in position 36, the third cysteine residue (amino acid residue 21) is linked to the sixth cysteine (amino acid residue 45) and the fourth cysteine residue (amino acid residue 25) is linked the seventh cysteine residue in position 47.

Plant defensins exhibiting direct antifungal activity have been reported from radish seed plant defensin (Terras et al., J. Biol. Chem., 267:15301-15309 (1992)). Inducible isoforms of defensins have also been reported in fungal pathogen-infected tissue (Terras et al., Plant Cell 7:573-588 (1995)). Dimeric forms of defensins have also been reported. Antifungal activity of some defensins are sensitive to calcium and potassium ions in the 1 and 50 millimolar range, respectively.

Thionins are 45 to 47 amino acids in length and possess six or eight disulfide linked cysteines. An eight cysteine residue thionin disulfide linkage pattern is reported to be X₂C₂X₇CX₃CX₁₀CXCX₇CX₆, where the first cysteine residue (amino acid residue 3) is linked to the last cysteine (amino acid residue 41), the second cysteine residue (amino acid residue 4) is linked to the seventh cysteine residue (amino acid residue 33), the third cysteine residue (amino acid residue 12) is linked to the sixth cysteine residue in position 31 and the fourth cysteine residue (amino acid position 16) is linked to the fifth cysteine residue in position 21. A six cysteine residue thionin disulfide linkage pattern has been reported to be X₂C₂X₁₁CX₁₀CX₅CX₇CX₆, where the first cysteine residue (amino acid residue 3) is linked to the last (sixth) cysteine residue at position 40, the second cysteine residue (amino acid residue 4) is linked to the fifth cysteine residue (amino acid residue 32) and the third cysteine (amino acid residue 16) is linked to the fourth cysteine residue (amino acid residue 26).

Direct in vitro antifungal and antibacterial activities of thionin from wheat have been reported (Stuart and Harris, Cereal Chem. 19:288-300 (1942)). A pathogen-inducible thionin gene from Arabidopsis thaliana, Thi2.1, encodes a thionin protein which displays antifungal activity in planta when expressed transgenic plants from a constitutive promoter (Epple et al., Plant Cell 9:509-520 (1997)).

Phospholipid transfer proteins have been characterized as proteins that mediate the transfer of phospholipid moieties from liposomes to mitochondrial membranes. These proteins occur in two subgroups, the more common one consists of approximately 9 kilodalton proteins and the other of approximately 7 kilodalton proteins. Both forms contain 8 disulfide-linked cysteine residues. A 9 kilodalton subgroup disulfide linkage pattern has been reported to be X₂CX₉CX₁₃C₂X₁₉CXCX₂₂CX₁₃CX₃, where the first cysteine residue (amino acid residue 2) is linked to the sixth cysteine (amino acid residue 50), the second cysteine residue (amino acid residue 13) is linked to the third cysteine residue (amino acid residue 27), the fourth cysteine residue (amino acid residue 12) is linked to the seventh cysteine residue in position 72 and the fifth cysteine residue (amino acid position 48) is linked to the last cysteine residue in position 86.

There are several examples of phospholipid transfer proteins that have direct antifungal activity. A reported phospholipid transfer protein has been isolated from radish seeds (Terras et al., Plant Physiol. 100: 1055-1058 (1992)). A protein from onion seeds with homology to phospholipid transfer protein has been reported to have direct antifungal activity but no lipid transfer activity (Cammue et al., Plant Physiol. 109:445-455 (1995)). The antifungal activity of some phospholipid transfer proteins are sensitive to calcium and potassium ions in the 1 and 50 millimolar range, respectively.

The hevein-type class of antifungal proteins are chitin-binding proteins of about 40 amino acid residues in length. Many proteins of this class are made with carboxyterminal extensions of approximately 9 residues that are removed upon maturation. A eight cysteine residue disulfide linkage pattern has been reported to be X₂CX₈CX₄C₂X₅CX₆CX₄CX₃CX, where the first cysteine residue (amino acid residue 3) is linked to the fourth cysteine (amino acid residue 18), the second cysteine residue (amino acid residue 12) is linked to the fifth cysteine residue (amino acid residue 24), the third cysteine residue (amino acid residue 17) is linked to the sixth cysteine residue in position 32 and the seventh cysteine residue (amino acid position 36) is linked to the last cysteine residue in position 40.

Hevein is a protein constituent of rubber tree latex and exhibits weak antifungal activity (Van Parijs et al., Planta 183:258-264 (1991)). Homologues from the seeds of Amaranthus caudatus have been reported to exhibit significantly more potent antifungal activity (Broekaert et al., Biochemistry 31:4380-4314 (1992)). In vitro antifungal activity of this class of protein is sensitive to calcium and potassium ions in the 1 and 50 millimolar range, respectively.

The class of knottin-type proteins are 36 to 37 residues in length and contain 6 disulfide linked cysteine residues. A six cysteine residue disulfide linkage pattern has been reported to be XCX₆CX₈C₂X₃CX₁₀CX₂, where the first cysteine residue (amino acid residue 2) is linked to the fourth cysteine (amino acid residue 19), the second cysteine residue (amino acid residue 9) is linked to the fifth cysteine residue (amino acid residue 23), the third cysteine residue (amino acid residue 18) is linked to the sixth cysteine residue in position 32.

Knottin-type antifungal protein has been reported from the seeds of Mirabilis jalapa and was characterized as inhibiting a broad range of fungi (Cammue et al., J. Biol. Chem. 267:2228-2233 (1992)). In vitro antifungal activity of this class of protein is sensitive to calcium and potassium ions in the 1 and 50 millimolar range, respectively.

Ib-AMPs are a set of four different types of antimicrobial peptides that have been reported from the seeds of Impatients balsamina. These peptides are about 20 residues in length and are the smallest reported antifungal peptides. They contain 4 disulfide linked cysteine residues. These peptides have been reported to be encoded in a multipeptide precursor form containing 6 units of the antifungal peptide motif separated by conserved propeptide domains. A four cysteine residue pattern has been reported to be X₂C₂X₈CX₃C.

Direct antifungal activity of Ib-AMPs was demonstrated to be broad spectrum and sensitive to calcium and potassium ions in the 1 and 50 millimolar range respectively (Tailor et al., J. Biol. Chem. 272:24480-24487 (1997)).

MBP-1 is a protein isolated from maize that is 33 amino acid residues long and contains 4 disulfide linked cysteine residues. A four cysteine residue pattern has been reported to be X₆CX₃CX₁₃CX₃CX₄. MBP-1 has been reported to have antifungal activity against several fungi (Duvick et al., J. Biol. Chem. 25718814-18820 (1992)).

2S albumins are seed storage proteins found in many plants specied. A 2S albumin has been reported from radish seed with antifungal activity against several plant pathogens including Alternaria brassicola and Verticillium dahliae and some bacteria species (Terras et al., J. Biol. Chem. 267:15301-15309 (1992)).

A purified preparation of trypsin and chymtrypsin inhibitors from cabbage foliage has been reported to have antifungal activity in vitro. The inhibitors suppressed spore germination and germ tube elongation of two phytopathogenic fungi species, Botrytis cinerea and Fusarium solani, by causing the leakage of the intracellular content of the fungi (Lorito et al., Mol. Plant-Microbe Interact. 7:525-527 (1994)). A proteinase inhibitor clone has been reported from cabbage (Williams et al. Plant Physiol. 114:747 (1997)).

It has been reported that exposure of tobacco suspension culture cells to pathogenic fungi induce expression of proteinase inhibitor genes (Richauer et al. Plant Physiol. Biochem. 306:579-584 (1992)). The production of proteinase inhibitors is affected by the age of the cell culture. Lipoxygenase has also been reported to play a role in the regulation of plant defense reactions.

10. Nitrogen and Sugar Transportation

Membrane proteins can function as signal transducers or as transporters. Transport proteins (transporters or permeases) transfer molecules across cell membranes, which can have nutritional or informational value for the cell. Use of these transduction pathways by external signals such as sugars, nitrogenous compounds, or other metabolites is associated with the control of biochemical processes inside the cell. Molecules can be a nutrient and a regulatory signal at the same time, and biochemical pathways can be induced or repressed by metabolites. Transporters themselves can act as substrate inducible or catabolite repressable. This positive and negative regulation provides a mechanism by which the metabolism can respond to multiple sources of nutrients. Transport systems that are required in a given environmental or developmental condition are active.

Cells, tissues, and organs of higher plants can be characterized as autotrophic or heterotrophic. Autotrophic regions of the plant may be widely separated from heterotrophic regions, as is the case with leaves and roots, or adjacent, as with the epidermis and the underlying mesophyll cells in the leaf. Pathways involved with movement of photoassimilates and nitrogenous compounds from sites of photosynthesis (source) or assimilation, respectively, to the sites of storage or use (sinks) can be by direct, symplastic connections through plasmodesmata or through complex pathways involving membrane carriers (transporters) and plant vascular elements. Photoassimilate captured by photosynthesis are exported from the leaf in the forms of sucrose and amino acids in order to satisfy the biochemical needs of heterotrophic cells which specialize in important processes such as nutrient acquisition (roots) or reproduction (flowers, seed, fruit) or growth (sink leaves, stems). Partitioning of C and N assimilates plays a role in crop yield. Plant transporters involved in the movement of sugars and nitrogenous compounds are essential molecules for improving assimilate partitioning, which plays a crucial role in plant productivity and crop yield.

Reviews on transporters in plants include Buckhout and Tubbe, In: Photoassimilate Distribution In: Plants And Crops, Zamski and Schaffer, eds, Marcel Dekker Inc., New York, 229-260 (1996), Frommer et al., Transporters for nitrogenous compounds in plants. Plant Mo. Biol. 26:1651-1670 (1994).

Transporters mechanisms have been reported as follows. Active transport requires the input of energy and in primary active transport the transporter is directly coupled to an energy source, such as a membrane ATPase. Secondary active transport involves the indirect coupling of energy from a cotransporter, and an energetically uphill transport of one molecule that is linked to the energetically downhill transport of another. Symports refer to transport of both molecules in the same direction. Antiports refer to transport of molecules in opposite directions (Buckhout and Tubbe, In: Photoassimilate Distribution In: Plants And Crops, Zamski and Schaffer, eds., Marcel Dekker Inc., New York, 229-260 (1996)).

Many of the reported sugar transporters found in plants, animals, and bacteria are catalytically, mechanistically, and structurally similar. They are integral membrane proteins with at least 12 reported hydrophobic transmembrane segments (Buckhout and Tubbe, In: Photoassimilate Distribution In: Plants And Crops, Zamski and Schaffer, eds., Marcel Dekker Inc., New York, 229-260 (1996)). Based on genes encoding these proteins from bacteria, yeast, mammals, and plants a general structure has been reported. The amino acid sequence is not necessarily conserved in transporters (Buckhout and Tubbe, In: Photoassimilate Distribution In: Plants And Crops, Zamski and Schaffer, eds., Marcel Dekker Inc., New York, 229-260 (1996)).

Triose sugars are the initial stable products of photosynthesis and their net efflux from the chloroplast provides carbon and energy for metabolic processes in the cell. Triose sugar transport across the inner chloroplast membrane is catalyzed by a carrier that is coupled to Pi counter transport, linking the metabolic requirements of the cell to the synthetic machinery of the chloroplast (Flugge and Heldt 1991; Heldt et al., Plant Physiol. 95:341-343 (1991)). This triose phosphate-Pi transporter (known as the phosphate translocator) has been reported to be an antiporter. This transporter has also been reported to control the levels of triose-phosphate and Pi in the cytosol and chloroplasts of photosynthesizing cells. These metabolites in turn are associated with the regulation of starch biosynthesis and sucrose synthesis and diurnal partitioning of photoassimilate between starch and sucrose in photosynthetically active tissues (Preiss, In: The Biochemistry of Plants, Stumpf and Conn, eds., Vol. 14, Preiss, ed., 181-254 (1988)).

Phosphate translocators from different plant species and plant cell types can differ in their substrate specificities, due to the specialization of cell types in a tissue or species. C3 plants have been reported to have phosphate translocators in their chloroplasts with highest specificities to triose-3-phosphates (3-PGA) versus triose-2-phosphates (PEP) (Fliege et al., Biochim. Biophys. Acta, 502:232-247 (1978)). C4 plant phosphate translocators transport PEP and 2-PGA as well as 3-PGA, facilitating C4 photosynthesis and the specialization of bundle sheath and mesophyll cells for 3-PGA and PEP transport, respectively (Ohnishi et al., Plant Physiol. 91:1507-1511 (1989)). Crassulacean acid metabolism (CAM) plants have phosphate translocators which have been reported to transport 3-PGA or PEP (Neuhaus et al., Plant Physiol. 87:64-68 (1988)). Phosphate translocators in plastids other than chloroplasts have been reported to have substrate specificities for hexose sugars, such as in maize kernel amyloplasts (glucose-1-P or glucose-6-P; Overlach et al., Plant Physiol. 101:1201-1207 (1993); Neuhaus et al., Plant Physiol. 101:573-578 (1993)).

A gene for a phosphate translocator protein was reported from spinach (Flugge et al., 1989). In addition, genes in pea (Willey et al., Planta 183:451-461 (1991)), potato (Schulz et al., Mol. Gen. Genet. 238:357-361 (1993)), maize, and Flaveria (Fischer et al., Plant J. 5:215-226 (1994)) have been reported. Deduced amino acids of these reported genes were 85 to 87% identical and encoded 35-37 kDa proteins. The reported functional translocators have been dimers and structural modeling from these cloned genes suggests that two hydrophilic transport channels are formed from a total of 12 transmembrane domains. The potato gene is not expressed in other tissues than leaves. Antisense experiments in potato show that reducing the phosphate translocator expression inhibits chloroplastic export of triose phosphate, and increases the amount of photoassimilate partitioned into starch (Riesmeier et al., Proc. Natl. Acad. Sci. (USA) 90:6160-6164 (1993)).

Both active and passive transport has been reported for sugar transport across the plasma membrane for cell to cell transport. Passive transport has been reported to involve transporter proteins, but direction of transport is determined solely by concentration gradient of the substrate, not by energy coupled transport (active). Active transport has been reported (Bush, Annu. Rev. Plant Physiol. Plant Mol. Biol. 44:513-542 (1993)). Active transport is driven by the electrochemical gradient across the plasma membrane established by a proton (H⁺) pumping ATPase, transporting H⁺ out of the plant cell and creating a pH gradient (acid outside). In reported cases to date, active uptake of sugars across the plasma membrane utilizes an H⁺-sugar symporter, with examples being the sucrose symporter and glucose symporter (Buckhout and Tubbe, In: Photoassimilate Distribution In: Plants And Crops, Zamski and Schaffer, eds., Marcel Dekker Inc., New York, 229-260 (1996)).

Many plants utilize sucrose as the chemical form to distribute photosynthetically derived energy and carbon to nonphotosynthetic organs. Long distance transport of sucrose through the plant has been reported to be catalyzed by a series of partial reactions ranging from diffusion between mesophyll cells in the leaf to active transport of sucrose into the sieve tube-companion cell complex, a step in phloem loading. Carrier-mediated transport of sucrose in phloem loading and this phenomenon has been reported in a variety of tissues and species (Sovonick et al., Plant Physiol. 54:886-891 (1974); Maynard and Lucas, Plant Physiol. 70:1436-1443 (1982); Lemoine et al., Plant Physiol. 86:575-580 (1988); Bush, Annu. Rev. Plant Physiol. Plant Mol. Biol. 44:513-542 (1993)). It has been reported that phloem loading of sucrose across the plasma membrane (into the sieve tube-companion cell) is carrier mediated by coupled symport with protons (Buckhout and Tubbe, In: Photoassimilate Distribution In: Plants And Crops, Zamski and Schaffer, eds., Marcel Dekker Inc., New York, 229-260 (1996)). An in vitro assay for sucrose transport has been reported (Bush, Plant Physiol. 89:1318-1323 (1989); Buckhout, Planta 178:393-399 (1989)).

Reported characteristics of sucrose transporters from isolated plasma membranes include saturable uptake, Kₘ for sucrose ranging from 0.5 to 2 mM sucrose, and sucrose accumulating in isolated vesicles to a concentration of up to two to five times greater than the surrounding media. A negative membrane potential has been reported to drive sucrose transport. The stoichiometry for sucrose/H⁺ was measured at nearly 1:1 (Buckhout and Tubbe, In: Photoassimilate Distribution In: Plants And Crops, Zamski and Schaffer, eds., Marcel Dekker Inc., New York, 229-260 (1996)). Specificity of the sucrose symporter for sucrose has been reported for in vivo for phloem transport in sugar beet (Fondy et al., Plant Physiol. 59:953-960 (1977)) and maize (Giaquinta, Annu. Rev. Plant Physiol. 34:347-387 (1983)). Uptake of sucrose into soybean protoplasts and sugar beet leaf disks has been reported to be inhibited by 100 fold maltose excess (Maynard and Lucas, Plant Physiol. 70:1436-1443 (1982)). Studies with sucrose derivatives and inhibition experiments in soybean cotyledons report that the glucose moeity of sucrose is solely responsible for the substrate recognition (Hitz et al., J. Biol. Chem. 261:11986-11991 (1986)). Sucrose has been reported the preferred substrate for a sucrose symporter with minor differences in substrate specificity in different tissues or species (Buckhout and Tubbe, In: Photoassimilate Distribution In: Plants And Crops, Zamski and Schaffer, eds., Marcel Dekker Inc., New York, 229-260 (1996)).

A sucrose transporter protein has been reported using chemical labeling of a 62 kDa protein from a preparation of plasma membranes from soybean cotyledons at a developmental stage where sucrose is actively imported (Ripp et al., Plant Physiol. 88:1435-1445 (1988)). Antibodies raised against this protein recognized antigens on the sieve tube plasma membrane of spinach leaves (Warmbrodt et al., Planta 180:105-115 (1989)), and antigens of the plasma membrane of companion cells of soybean cotyledons (Grimes et al., Plant Cell 4:1561-1574 (1992)). A gene was isolated and sequenced for this 62 kDa protein and shows little sequence identity to other sucrose symporters (Grimes et al., Plant Cell 4:1561-1574 (1992). A reported sucrose symport gene from a spinach cDNA library isolated by complementation of a yeast mutant encodes a protein with predicted mass of 55 kDa, has 12 hydrophobic regions typical of plasma membrane symport proteins, but no sequence similarity to other sugar cotransporters (Riesmeier et al., EMBO J. 11:4705-4713 (1992)). A potato cDNA has also been reported and isolated by a similar approach, with 60% amino acid identity to the spinach gene. Expression of the potato gene was reported in leaf minor veins, reported regions of phloem unloading. The reported gene is strongly expressed in source leaves and roots with little or no expression in sink leaves and stems and tubers and flowers (Riesmeier et al., EMBO J. 13:1-7 (1994)). Antisense reduction of this gene in potatoes resulted in transgenic potato plants that were growth retarded in juvenile stages and that accumulated carbohydrates in leaves, along with reductions in export of sucrose from source leaves and reduction in tuber development (Riesmeier et al., EMBO J. 13:1-7 (1994)). A role for sucrose symporter in phloem loading and export of carbohydrates from source leaves in defining photoassimilate partitioning and flow of carbon from source to sink in plants has been reported.

Other carbon forms in some plant species may be used to transport energy. Sorbitol, is a primary photoassimilate in some members of Rosaceae, such as Malus, Prunus, and Pyrus (Bieleski, Aust. J. Plant Physiol. 4:11-24 (1977)). Sorbitol uptake into phloem has been reported to be carrier mediated (Buckhout and Tubbe, In: Photoassimilate Distribution In: Plants And Crops, Zamski and Schaffer, eds., Marcel Dekker Inc., New York, 229-260 (1996)). Other plants may transport mannitol as photoassimilate (Loescher, Physiol. Plant 70:553-557 (1987)), and others may utilize sucrosylgalacto sides (raffinose, stachyose, and verbascose; Van Bel, Annu. Rev. Plant Physiol. Plant Mol. Biol. 44:253-281 (1993)), but little is known about their transport.

Glucose and other hexoses are intermediates in the storage of carbon and energy and hexose transport has been reported to play a role in heterotrophic tissues such as roots, stems, and reproductive organs (seeds, fruit) that form metabolic sinks. In Zea mays, sucrose phloem unloading occurs in the apoplast, where invertases hydrolyze to glucose and fructose, both of which are transport sugars (Doehlert and Felker, Physiol. Plant. 70:51-57 (1987)). First studies on glucose uptake indicated a carrier mediated process, and studies initially in Chlorella (Komor, E., FEBS Lett. 38:16-18 (1973)), indicated a glucose symport with +, and substrate specificity for other hexoses as well.

Plasma membrane transport of hexose in higher plants has been reported (Komor, In: Encyclopedia of plant physiology, new series, Pearson and Zimmermann, eds., Springer-Verlag, Berlin, 635-676 (1982); Rausch, Physiol. Plant 82:134-142 (1991)). In Streptanthus cells two transport systems have been reported, one with affinity and specificity for glucose, the other transporting either glucose or fructose (Rausch et al., Plant Physiol. 85:996-999 (1987)). Plasma membrane isolation studies have investigated the specificity of transport for hexoses with sugar beet cells being more specific for glucose (Zamski and Wyse, Plant Physiol. 78:291-295 (1985)), and other species having variable specificities for hexoses. A family of genes for hexose transport, with differential expression of the genes in each cell type defining hexose transport have been reported (Sauer and Tanner, Bot. Acta 106:277-286 (1993)).

A hexose symporter gene (HUP1) from Chlorella with typical 12 membrane domains has been cloned with similar kinetic characteristics and substrate preferences to those in vivo, when expressed in yeast (Sauer et al., Proc. Natl. Acad. Sci. (USA) 87:7949-7952 (1990); Sauer et al., EMBO J. 9:3045-3050 (1990)). Since then several genes ranging in amino acid identity from 45-80% have been reported from Arabidopsis (STP1) and other species (Sauer and Tanner, Bot. Acta 106:277-286 (1993); Bugos et al., Plant Physiol. 103:1468-1470 (1993)). Certain gene family members have been reported to be strongly expressed in roots in Arabidopsis and tobacco. Other gene family members have been reported to be expressed only in leaves (Sauer et al., EMBO J. 9:3045-3050 (1990)).

The vacuole provides temporary storage for photoassimilate in photosynthetically active mesophyll cells or a compartment for long term storage in organs such as tap roots or hypocotyls of beet or storage cells of sugar cane (Buckhout and Tubbe, In: Photoassimilate Distribution In: Plants And Crops, Zamski and Schaffer, eds., Marcel Dekker Inc., New York, 229-260 (1996)). Transport of sugars across the vacuolar membrane has been reported to be passive or active, the latter driven by a H⁺ gradient generated by an ATPase or PPiase.

In dormant tubers of Japanese artichoke (Stachys) it has been reported that stachyose is stored against a concentration gradient in vacuoles (Keller and Matile, J. Plant Physiol. 119:369-380 (1985)). It has further been reported that uptake of both sucrose and stachyose into the vacuole is active by means of a H⁺-sugar antiporter (Greutert and Keller, Plant Physiol. 101:1317-1322 (1993)).

Glucose transport into isolated pea mesophyll vacuoles has been reported as H⁺-driven glucose antiport (Guy et al., Plant Physiol. 64:61-64 (1979)). Similar mechanism has been suggested for sugar cane protoplasts (Thom et al., Plant Physiol. 69:1320-1325 (1982)), and maize coleoptile tonoplasts (Rausch et al., Plant Physiol. 85:996-999 (1987)).

Accumulation of sucrose in sugar beets and sugar cane vacuoles, because of huge economic importance has been given more study. Sucrose transport into sugar beet vacuoles has been reported to be by a sucrose-H⁺ antiport (Willenbrink and Doll, Planta 147:159-162 (1979); Briskin et al., Plant Physiol. 78:871-875 (1985)). In sugar cane, vacuole transport is also consistent with sucrose-H⁺ antiport. In barley mesophyll cells and other C3 plants, much of the photoassimilate produced is stored in leaf mesophyll cells soon after photosynthesis as sucrose in the vacuole. It has been reported that this transport of sucrose occurs by passive transport with a facilitated carrier (facilitated diffusion) (Martinoia, Bot. Acta 105:232-245 (1992)), and that sink cells utilize active transport for vacuolar storage of sucrose with source cells using facilitated diffusion.

The growth of a plant is limited by the nutrient present in limiting amounts. Nitrogen is often a limiting nutrient in the soil.

Uptake can involve three major sources of nitrogen including nitrate, ammonium, and to a lesser extent amino acids occurs if the nitrogenous substance is not permeable, across the membrane in the outer cell layers of the root (root hairs and cortex). Reduction, fixation and use may take place directly or in neighboring cells or transported to other organs (Frommer et al., Plant Mo. Biol. 26:1651-1670 (1994)). This translocation can occur by apoplastic transport through the cortex, then symplastically to the stele, and then transferred to the xylem by additional transport systems (Pitman, Annu Rev Plant Physiol. 28:71-88 (1977)).

Translocation and processing of nitrogenous compounds into amino acids and proteins is dependent on the plant species. Nitrate may be taken up and reduced directly in the root or it can be transported through the xylem to the leaves where photosynthate can be used to make amino acids. Ammonium assimilation has been reported to occur directly in the root. It has been reported that reduced nitrogen is transported in the form of amino acids, amides and ureides within the xylem and phloem (Frommer et al., Plant Mo. Biol. 26:1651-1670 (1994)).

Reduced nitrogen is stored transiently as vegetative storage protein or storage proteins in seeds, and this reduced nitrogen is reallocated during plant development from exporting sources such as roots, leaves, or endosperm or cotyledons (in early development). Transport may occur by xylem or phloem and may involve several types of nitrogenous compounds (amino acids) (Frommer et al., Plant Mo. Biol. 26:1651-1670 (1994)). Carrier mediated transport has been reported for uptake and transfer of nitrate, ammonium, and amino acids in plants.

Nitrate uptake has been reported to be mediated by specific transport systems and acts as a symport with at least two protons (Doddema and Telkamp, Physiol Plant. 45:332-338 (1979); Glass et al., Plant Physiol. 99:456-463 (1992); Goyal and Huffaker, Plant Cell Environ. 9:209-215 (1986)). Mutants and gene cloning studies have identified an amino acid transporter/pump with membrane spanning domains from Aspergillus (CRNA; Unkles et al., PNAS 88:204-208 (1991)), and an amino acid transporter/pump in Arabidopsis, an integral membrane protein (CHL1; Tsay et al., Cell 72:705-713 (1993). In higher plants, absorbed nitrate or nitrite is either reduced in the roots and exported in the form of amino acids or transported to the leaves, where it is reductively assimilated to produce amino acids.

Under agronomic conditions that inhibit nitrification, such as rice cultivation or cold or acidic soils for other species, ammonium may be the most prevalent source of nitrogen (Wang et al., Plant Physiol. 103:1249-1258 (1993)). Ammonium occurs in bound forms in the soil and requires efficient release systems. Plant systems in place for uptake including a saturable carrier mediated system at low ammonium concentrations and a linear diffusive component at elevated ammonium concentrations have been reported (Fried et al., Physiol Plant. 18:313-320 (1965); Wang et al., Plant Physiol. 103:1259-1267 (1993)). A high affinity ammonium transport system has been reported from plants (Ninneman et al., EMBO J. 13:3464-3471 (1994)). Similar to nitrate transport, ammonium transport has been reported to involve transporters for cortex uptake, transfer and release into/from the vessels and specific retrieval systems to prevent loss in the form of ammonia (Frommer et al. Plant Mo. Biol. 26:1651-1670 (1994)).

Plants which undergo symbiosis with nitrogen fixing organisms accept dinitrogen reduced in the nitrogen fixing organism and transfer to the plant cytoplasm. Ammonium is a major transport form for movement across the rhizobium (dinitrogen fixers) membranes and this occurs by diffusion into the more acidic plant cell wall (Kleiner, In: Alkali Cation Transport Systems in Procaryotes, Bakker, ed., CRC Press, London, 379-396 (1993)). Ammonium taken up or produced by nitrogen fixation feeds into amino acid biosynthesis and these amino acids can by exported by the vascular tissue to organs and cells that are dependent on external supply (Frommer et al., Plant Mo. Biol. 26:1651-1670 (1994)).

Export of amino acids have been reported via the xylem in species where amino acid biosynthesis occurs in the roots. Roots have also been reported to have uptake systems for amino acids and an active transport system (Schobert and Komor, Planta 177:342-349 (1989)). The major amino acids reported in root exudates have been glutamine (Allen and Raven, J. Exp. Bot. 38:580-596 (1987)), asparagine and glutamate (Shelp, J. Exp. Bot. 38:1619-1636 (1987)).

In barley it has been reported that the concentration of amino acids in the leaf mesophyll cells mimics that found in the phloem sap (Winter et al., Plant Physiol. 99:996-1004 (1992)). In addition the composition of xylem sap and phloem sap has been reported to be similar indicating that loading of phloem is not selective and that a transport system with broad substrate specificity exists (Frommer et al., Plant Mo. Biol. 26:1651-1670 (1994).

It has been reported that amino acids arrive to developing seeds by phloem, and that a xylem to phloem transfer can occur before phloem unloading, at least in species where root tissue is responsible for most amino acid synthesis (Thorne, Annu Rev Plant Physiol 36:317-343 (1985)). Embryo tissue symplastically isolated from the maternal tissue in developing seeds, and amino acids have to pass the apoplastic space before entering seeds, suggesting possible roles for transporters. Arabidopsis transporter genes AAP1 and AAP2 are expressed in developing seed pods both a role in phloem unloading or assimilate transfer (Kwart et al., Plant J. 4:993-1002 (1993)). AAP1 and AAP2 are also expressed in the vascular tissue of developing cotyledons and it has been reported that these play a role in supplying germinating seedlings with reduced nitrogen (Frommer et al. Plant Mo. Biol. 26:1651-1670 (1994)).

Studies in Chlorella have reported that amino acids are inducible and divided into one specific for basic amino acids, one specific for neutral amino acids, and one system for a number of amino acids (Langmuller and Springer-Lederer, Planta. 120:189-196 (1974)). In Nicotiana and Commelina, a low affinity and high affinity transport system have been reported (Borstlap, In: Fundamental Ecological and Agricultural Aspects of Nitrogen Metabolism in Higher Plants, Lambers, Neeteson, and Stulen, eds. Martinus Nijhoff Publishers, Dordrecht, 115-117, (1986); van Bel et al., Planta. 186:518-525 (1992)), with a tissue specific in function, i.e., different from mesophyll to the vascular tissue. In sugar beet, the uptake of several amino acids has been reported to be coupled to cotransport of protons (Li and Bush, Plant Physiol. 94:268-277 (1990)). Transport systems are associated with uptake into roots, mobilization from roots in the xylem, unloading of phloem and possibly transfer from xylem to phloem. Sink organs have been reported to require two systems, one in maternal tissue for phloem unloading and one for uptake into the developing embryo or seed tissue.

Several gene families from Arabidopsis have been identified as encoding amino acid transporters (Frommer et al., PNAS. 90:5944-5948 (1993)). These include at least two reported gene families with integral membrane proteins able to mediate amino acid transport, with the first group being named amino acid permease (AAP) ((Frommer et al., PNAS. 90:5944-5948 (1993); Kwart et al., Plant J. 4:993-1002 (1993)). Two members of this family have been reported to contain 9-12 membrane domains and encode polypedtides of 53 kDa (Frommer et al. Plant Mo. Biol. 26:1651-1670 (1994)). Amino acid permeases from bacteria, animals, and fungi can be grouped into several categories including those specific for neutral and acidic amino acids and proline (Kanai and Hediger, Nature 360:467-471 (1992)), the cationic and aromatic amino acid transporters (Heatwole and Somerville, J. Bact. 173:108-115 (1991); Honore and Cole, Nucl. Acids Res. 18:653 (1990)), those related to the plant AAP family (see above), and the transporters which contain ATP-binding cassettes (Higgins et al., J. Bioenerg. Biomembr. 22:571-592 (1990)).

Chlorella has been reported to have up to seven transport systems with different substrate specificities (Frommer et al. Plant Mo. Biol. 26:1651-1670 (1994)). In higher plants the numbers of systems may range from one to several. Three distinct transport systems, one for neutral including glutamine, asparagine, and histidine, one for acidic and one for basic amino acids, were reported in sugar cane suspension cells (Wyse and Komor, Plant Physiol. 76:865-870 (1984)). Reported Arabidopsis carriers in the AAP family differ in substrate specificity with respect to basic amino acids, but have a general or broad specificity which has been reported to cover the transport of the major components found in xylem and phloem (Frommer et al. Plant Mo. Biol. 26:1651-1670 (1994)).

A direct export of small peptides has been reported to enhance the transport efficiency of tissues which store proteins. Transport activities for peptides were reported in a variety of plant tissues (Higgins and Payne, Planta 138:217-221 (1978)), especially localized in tissues like germinating seedlings. Peptide transporter genes have been reported from yeast (Dubois and Grenson, Mol. Gen. Genet. 175:67-76) and Arabidopsis.

F. Phenolic Metabolism

1. Shikimate Pathway

The shikimate, or common aromatic, pathway is reported to play a role in the production of precursors for aromatic compounds in microbes and plants (Herrmann, Plant Cell 7:907-919 (1995); and Herrmann, Plant Physiology 107:7-12 (1995)). As used herein, the term shikimate pathway is used generically to refer to pathways that lead to the biosynthesis of chorismate, phenylalanine, tyrosine, and tryptophan.

In fungi, except for the first enzyme of the shikimate pathway, all reactions are catalyzed by a pentafunctional arom complex that produces chorismate. In bacteria and plants, enzymes of the shikimate pathway are monofunctional. In microbes, the pathway serves primarily for the production of aromatic amino acids for protein biosynthesis. In plants the pathway generates not only phenylalanine, tyrosine, and tryptophan, but also other aromatic compounds derived from chorismate, the end product of the shikimate pathway, or from phenylalanine, tyrosine, and tryptophan (Dewick, Natural Product Reports 11:173-203 (1994)).

A plastidic location for the shikimate pathway and the terminal pathways to phenylalanine, tyrosine, and tryptophan has been reported based on biochemical (Bickel and Schultz, Phytochemistry 18:498-499 (1979)) and molecular analysis (Schmid and Amrhein, Phytochemistry 39:737-749 (1995); Della-Cioppa et al., Proc. Natl. Acad. Sci. (U.S.A.) 83:6873-6877 (1986); and Schmid et al., Plant Journal 2:375-383 (1992)). Cytosolic enzyme activities for the first enzyme of the shikimate pathway and the first enzyme of the pathways leading to tyrosine and phenylalanine have also been reported. A “dual pathway” hypothesis has been proposed, with a plastidic shikimate pathway responsible for the production of aromatic amino acids, and a cytosolic one responsible for the production of chorismate required for the synthesis of secondary metabolites.

In plants, the formation of chorismate from the shikimate pathway is reported to consist of seven reactions catalyzed by six enzymes. Chorismate is subsequently converted to phenylalanine or tyrosine in three reactions, and to tryptophan in five reactions.

The first reported step in the production of chorismate is reported to begin with the condensation of phosphoenolpyruvate (“PEP”) and erythrose-4-phosphate (“E4P”) to form the seven carbon sugar, 3-deoxy-D-arabino heptulosonate 7-phosphate (“DAHP”). This reaction is catalyzed by the enzyme 3-deoxy-D-arabino heptulosonate 7-phosphate synthase (also referred to as DAHP synthase, and DAHPS (E.C. 4.1.2.15)). Multiple isoenzymes of DAHPS are reported to exist in plants. Enzyme activity has been reported in cytosol and plastid (Ganson et al., Plant Physiology 82:203-210 (1986)). The cytosolic form of the enzyme is reported to have a broad substrate specificity and to be associated with cytosolic functions other than the production of DAHP (Doong et al., Physiologia Plantarum 84:351-360 (1992)). A plastidic DAHPS isoenzyme has been reported to be inhibited by arogenate, a post-shikimate pathway intermediate of plant phenylalanine and tyrosine biosynthesis (Doong et al., Plant Cell and Environment 16:393-402 (1993)). A hysteretic enzyme whose activity is enhanced by tyrosine and tryptophan (Doong et al., Plant Cell and Environment 16:393-402 (1993); Herrmann, Plant Physiology 107:7-12 (1995)). Genes encoding plastidic DAHPS have been reported and contain a chloroplast transit sequence.

Two types of plastidic DAHPS, having about 80-85% sequence identity at the amino acid level, have been reported in plants (Zhao and Herrmann, Plant Physiology 100: 1075-1076 (1992)). One enzyme has been reported to increase in response to environmental changes such as nutritional stress, light, wounding, and pathogen attack at the transcriptional level which leads to an accumulation of the enzyme and an increase in activity (Keith et al., Proc. Natl. Acad. Sci. (U.S.A.) 88:8821-8825 (1991); Görlach et al., Plant Molecular Biology 23:697-706 (1993); Umeda et al., Plant Molecular Biology 25:469-478 (1994); Guyer et al., Proc. Natl. Acad. Sci. (U.S.A.) 92:4997-5000 (1995); Henstrand et al., Plant Physiology 98:761-763 (1992); Dyer et al., Proc. Natl. Acad. Sci. (U.S.A.) 86:7370-7373 (1989); Jones et al., Plant Physiology 108:1413-1421 (1995); Conn and McCue, Studies in Plant Sciences 4:95-102 (1994); Görlach et al., Proc. Natl. Acad. Sci. (U.S.A.) 92:3166-3170 (1995)). In tomato, an inducible DAHPS isoenzyme is reported to be expressed at higher levels in roots and flowers, lower levels in stems, and lowest levels in leaves and cotyledons. A second class of plastidic DAHPS, which is constitutively expressed, accumulates to higher levels in flowers and lower levels in the stems of tomato (Görlach et al., Planta 193:216-223 (1994)).

A second enzyme of the shikimate pathway is encoded by dehydroquinate synthase (“DHQS” (E.C. 4.6.1.3)). DHQS has been reported from a plant source, and both biochemical and molecular evidence indicate that this enzyme is located in the chloroplast (Bickel and Schultz, Phytochemistry 18:498-499 (1979); Bischoff et al., Plant Molecular Biology 31:69-76 (1996)). DHQS mRNA has been reported to be expressed at high levels in roots, low levels in leaves, and at moderate levels in other organs. It has also been reported that DHQS mRNA levels increase about 5-fold in response to fungal elicitor (Bischoff et al., Plant Molecular Biology 31:69-76 (1996)). Dehydroquinate synthase catalyzes the conversion of 3-deoxy-D-arabino-heptulosonate 7-phosphate (DAHP) to 3-dehydroquinate (Yamamoto and Minamikawa, J Biochem (Tokyo) 80:633-635 (1976)).

The third and fourth reactions of the plant shikimate pathway are catalyzed by a bifunctional enzyme: dehydroquinase (E.C. 4.2.1.10) and shikimate dehydrogenase (“DHQ/SDH” (E.C. 1.1.1.25)). Partial cDNA sequences coding for the carboxy terminal portion of the protein have been isolated from pea and tobacco (Deka et al., FEBS Letters 349:397-402 (1994); Bonner and Jensen, Biochemical Journal 302:11-14 (1994)). It has also been reported that DHQ/SDH activity in pea is subject to light regulation (Rothe and Hengst, Z. Pflanzenphysiol. 101:223-232 (1981)).

A product of the bifunctional DHQ/SDH is converted to shikimate-3-phosphate by shikimate kinase (“SK” (E.C. 2.7.1.71)). Shikimate kinase is reported to convert shikimate to shikimate 3-phosphate (Griffin and Gasson, DNA Seq. 5:195-197 (1995)). In tomato, the SK gene has been reported to be encoded by only a single gene per haploid genome. SK contains a plastid or chloroplast transit sequence, and has been reported to be imported into chloroplasts in vitro (Schmid et al., Plant Journal 2:375-383 (1992)). SK is reported to be induced by fungal elicitors. It has also been reported that under fungal elicitation in tomato cell culture, SK transcript accumulation reaches a peak approximately at the same time as phenylalanine ammonia lyase message, and earlier than peak transcript accumulation for other enzymes of the shikimate pathway (Bischoff et al., Plant Molecular Biology 31:69-76 (1996); Görlach et al., Proc. Natl. Acad. Sci. (U.S.A.) 92:3166-3170 (1995)).

The penultimate reported reaction of the shikimate pathway is the conversion of shikimate 5-phosphate to 3-enolpyruvyl shikimate 5-phosphate (“EPSP”), catalyzed by 3-enolpyruvyl shikimate 5-phosphate synthase (also referred to as EPSP synthase or “EPSPS” (E.C. 2.5.1.19)). EPSPS has been characterized (Herrmann, Plant Cell 7:907-919 (1995); Herrmann, Plant Physiology 107:7-12 (1995); Dewick, Natural Product Reports 11: 173-203 (1994)). It has been reported that EPSPS, synthesized in vitro from plant cDNA can be imported into isolated chloroplasts (Della-Cioppa et al., Proc. Natl. Acad. Sci. (U.S.A.) 83:6873-6877 (1986)). EPSPS activity and transcripts are reported to respond to developmental and environmental conditions. Two isozymes have been reported in maize, one of which changes in response to the growth stage of cell cultures (Forlani et al., Plant Physiology 105:1107-1114 (1994)). In tomato, EPSPS transcript expression patterns in various organs essentially parallel that of the inducible DAHPS (Görlach et al., Planta 193:216-223 (1994)). EPSPS transcripts are also reported to accumulate in response to nutritional stress (Guyer et al., Proc. Natl. Acad. Sci. (U.S.A.) 92:4997-5000 (1995)) and fungal elicitors (Görlach et al., Proc. Natl. Acad. Sci. (U.S.A.) 92:3166-3170 (1995)). In Euglena gracilis, plastidic EPSPS is reported to be differentially expressed in response to light (Reinbothe et al., Mol. Gen. Genet. 245:616-622 (1994)).

The final reported reaction of the shikimate pathway is catalyzed by chorismate synthase (“CS” (E.C. 4.6.1.4)). CS catalyses the conversion of 5-enolpyruvylshikimate 3-phosphate (EPSP) to form chorismate (Henstrand et al., Mol. Microbiol. 22:859-866 (1996)). A cDNA for CS has been reported from tomato (Braun et al., Planta 200:64-70 (1996)). Like DAHPS, CS has been reported to be encoded by two genes, and exhibit differential induction by fungal elicitors (Görlach et al., Plant Mol. Biol. 23:707-716 (1993)) and varying transcript levels in plant organs (Görlach et al., Planta 193:216-223 (1994)). A reported CS cDNA contains plastid transit sequences and a CS protein is reported to contain an unclipped transit sequence that is enzymatically inactive (Henstrand et al., Plant Physiology 108:1127-1132 (1995)).

An end product of the shikimate pathway, chorismate, is converted to phenylalanine or tyrosine by three enzymatic reactions that are reported to differ only in the final step. In addition to protein synthesis, phenylalanine and tyrosine are also reported to be the precursors to several secondary metabolites. Since these secondary metabolites are often produced in the cytosol, multiple subcellular locations for this pathway have been reported.

Chorismate mutase (“CM” (E.C. 5.4.99.5)), the first reported enzyme in the terminal pathway leading to phenylalanine and tyrosine, has been characterized at the biochemical and molecular level. Higher plants generally have two CM isozymes, and two activities, one activity, CM1, has been reported in the plastid, while the second, CM2, is reported in the cytosol (d'Amato et al., Planta 162:104-108 (1984); Benesova and Bode, Phytochemistry 31:2983-2987 (1992)). Plant CM, a monofunctional enzyme, is reported to not exhibit substantial sequence homology to microbial CM, which is part of a bifunctional protein (Eberhard et al., Plant Journal 10:815-821 (1996)). Like DAHPS and CS, the two reported isozymes of CM exhibit differential responses to environmental factors. In Arabidopsis, transcripts encoding CM1 are reported to accumulate at higher levels in roots, with lower levels accumulating in leaves (Eberhard et al., Plant Journal 10:815-821 (1996)). CM1 transcripts have been reported to be induced by pathogens (Eberhard et al., Plant Journal 10:815-821 (1996)). Plastidic CM1 activity has been reported to increase in response to wounding in potato (Kuroki and Conn, Plant Physiology 89:472-476 (1989)). CM1 activity is also reported to be regulated by the aromatic amino acids; phenylalanine and tyrosine, which inhibit activity, and tryptophan which, activates it (Romero et al., Phytochemistry 40:1015-1025 (1995)). Cytosolic CM, CM2, has not been reported to show a similar response to environmental conditions reported for CM1 (Eberhard et al., Plant Journal 10:815-821 (1996); Kuroki and Conn, Plant Physiology 89:472-476 (1989); Romero et al., Phytochemistry 40:1015-1025 (1995)).

The product of CM, prephenate, is converted to arogenate by the action of prephenate aminotransferase (“PAT” (E.C. 2.6.1.-)). PAT has been reported from Anchusa officinalis (De-Eknamkul et al., Archives of Biochemistry and Biophysics 267:87-94 (1988)) and in tobacco cell culture (Bonner et al., Physiologia Plantarum 73:451-456 (1988)). A purified PAT has a native molecular weight of 220 kD and consists of heteromeric subunits with molecular weights of 44 and 57 kD, indicating an α₂β₂ subunit structure De-Eknamkul et al., Archives of Biochemistry and Biophysics 267:87-94 (1988)). PAT has been reported to be thermotolerant (Bonner and Jensen, Planta 172:417-423 (1987)), specific for prephenate, and capable of utilizing either aspartate or glutamate as the amino donor (De-Eknamkul et al., Archives of Biochemistry and Biophysics 267:87-94 (1988)). Greater than 90% of PAT activity is reported to be found in the plastid (Siehl et al., Plant Physiology 81:711-713 (1986)).

Arogenate, produced by PAT, is the last reported common intermediate of phenylalanine and tyrosine synthesis. Arogenate is converted to phenylalanine by the action of arogenate dehydratase (E.C. 4.2.1.-), and to tyrosine by the action of arogenate dehydrogenase (E.C. 1.3.1.43). Arogenate dehydratase has been partially purified from Sorghum. Sorghum arogenate dehydratase activity has been reported to be inhibited by its product, phenylalanine (Kᵢ 24 μM), and stimulated by tyrosine (Kₐ 2.5 μM) (Siehl and Conn, Archives of Biochemistry and Biophysics 260:822-829 (1988)). Arogenate dehydrogenase activity from Sorghum has been reported to be inhibited by tyrosine (Kᵢ 61 μM), but unaffected by phenylalanine (Connelly and Conn, Z. Naturforsch. Biosciences 41:69-78 (1986)). Both arogenate dehydrogenase and arogenate dehydratase are reported to have a Kₘ for arogenate of about 300 μM-350 μM (Connelly and Conn, Z. Naturforsch. Biosciences 41:69-78 (1986); Siehl and Conn, Archives of Biochemistry and Biophysics 260:822-829 (1988)).

Synthesis of tryptophan from chorismate has been reported to involve five reactions. These five reported reactions are conserved between microbes and plants. In addition to tryptophan, this pathway can lead to the biosynthesis of secondary metabolites including auxin, indole alkaloids, phytoalexins, cyclic hydroxamic acids, indole glucosinolates and acridone alkaloids. The functions of these secondary metabolites have been reported to include regulating plant growth, disease and insect resistance, and pollinator attractant.

Genes and/or cDNA's coding for each of the enzymes in the tryptophan synthetic pathway have been reported. It has also been reported that the tryptophan biosynthetic pathway is located in the plastid (Zhao and Last, Journal of Biological Chemistry 270:6081-6087 (1995); Radwanski and Last, Plant Cell 7:921-934 (1995)).

The first reported enzyme to be involved in the synthesis of tryptophan is anthranilate synthase (“AS” (E.C. 4.1.3.27)). This enzyme converts chorismate to anthranilate by elimination of the enolpyruvyl side chain, followed by an amino transfer from glutamine. AS has been reported to comprise non-identical subunits in an α₂β₂ structure (Romero et al., Phytochemistry 39:263-276 (1995)). In plants, multiple isozymes of AS have been reported to exist. AS has been reported to be feedback inhibited by tryptophan (Radwanski and Last, Plant Cell 7:921-934 (1995); Romero et al., Phytochemistry 39:263-276 (1995)). In Arabidopsis and R. graveolens, two cDNAs coding for the α subunit (ASA1 or ASα1 and ASA2 or ASα2, respectively) have been reported. The proteins encoded by these genes exhibit about 30-40% amino acid identity to an E. coli AS α subunit. ASA1 or ASα1 and ASA2 or ASα2 contain putative plastid transit sequences, and fall into two classes having approximately 70-80% amino acid identity. In Arabidopsis, three genes coding for the β subunit have been reported (ASB1, ASB2, ASB3) (Radwanski and Last, Plant Cell 7:921-934 (1995); Romero et al., Phytochemistry 39:263-276 (1995)).

The genes that constitute AS have been reported to be differentially expressed. The mRNA for ASA1 and ASB1 have been reported to be more abundant than the mRNA coding for other AS subunits. The ASA1 and ASB 1 genes are reported to be involved in a response to microbial attack (Radwanski and Last, Plant Cell 7:921-934 (1995); Romero et al., Phytochemistry 39:263-276 (1995); Schmid and Amrhein, Phytochemistry 39:737-749 (1995)). ASA1 and ASA2 have been reported to exhibit different organ expression (Radwanski and Last, Plant Cell 7:921-934 (1995)), and in R. graveolens, only ASα1 is reported to be feedback inhibited by tryptophan (Bohlmann et al., Plant Physiology 111:507-514 (1996)). An Arabidopsis mutant, trp5-1, has been reported to be defective in the feedback inhibition of AS by tryptophan. Free tryptophan levels in the leaf of the trp5-1 mutant have been reported to be three times higher than that of the wild type (Li and Last, Plant Physiol. 110:51-59 (1996)).

A phosphoribosyl moiety is attached to the amino group of anthranilate by phosphoribosyl anthranilate transferase (“PRAT” (E.C. 2.4.2.18)). A cDNA that encodes PRAT, termed PAT1, has been reported (Elledge et al., Proc. Natl. Acad. Sci. (U.S.A.) 88:1731-1735 (1991)). It has been reported that a PRAT enzyme activity is deficient in the trp1 mutant of Arabidopsis (Last and Fink, Science 240:305-310 (1988)). PRAT is reported to be encoded by a single gene in Arabidopsis (Rose et al., Plant Physiology 100:582-592 (1992)). Mutants in this gene are reported to reduce fertility and severe alleles are auxotrophs (Radwanski and Last, Plant Cell 7:921-934 (1995)).

Phosphoribosyl anthranilate is converted to enol-1-carboxyphenylamino-1-deoxyribulose 5-phosphate by the action of phosphoribosyl anthranilate isomerase (“PRAI” (E.C. 5.3.1.-)). In contrast to microbes, a plant PRAI is a monofunctional protein. This enzyme has been reported to be encoded by three genes in Arabidopsis, with 90% or greater amino acid identity. In fact, two of these genes are reported to differ by only a single amino acid (Li et al., Plant Cell 7:447-61 (1995)).

Indole-3-glycerol phosphate synthase (“IGPS” (E.C. 4.1.1.48)) produces an indole ring structure that is a precursor to tryptophan, auxin, and other indole-containing compounds in plants. In microbes, PRAI and IGPS activities occur in a bifunctional protein, in contrast to plants, where these activities are found as monofunctional enzymes. Utilizing E. coli mutants, a cDNA encoding Arabidopsis IGPS has been reported. Arabidopsis IGPS is reported to share low homology to its microbial counterparts (20-40% amino acid identity) (Li et al., Plant Physiology 108:877-878 (1995); Eberbhard et al., Biochemistry 34:5419-5428 (1995); Li et al., Plant Cell 7:447-461 (1995)).

The final reported reaction of tryptophan synthesis is catalyzed by tryptophan synthase (“TS” (E.C. 4.2.1.20)). TS consists of non-identical subunits, α and β. The α subunit produces indole, and the β subunit utilizes serine to produce tryptophan. A tryptophan synthase α subunit clone (TSA1) has been isolated from Arabidopsis, and it has been reported to share 30-40% identity with microbial TSα subunits (Radwanski et al., Mol. Gen. Genet. 248:657-667 (1995)). The reported β subunit shares 50-65% amino acid identity between microbes and plants (Schmid and Amrhein, Phytochemistry 39:737-749 (1995)). A cDNA coding for the TSβ subunit has been cloned from Arabidopsis (Berlyn et al., Proc. Natl. Acad. Sci. (U.S.A.) 86:4604-4608 (1989)). Two reported genes, TSB1 and TSB2, which exist in Arabidopsis and maize, exhibit homology (Last et al., Plant Cell 3:345-358 (1991); Wright et al., Plant Cell 4:711-719 (1992)). In Arabidopsis, TSB1 has been reported to be expressed at higher levels than TSB2 (Last et al., Plant Cell 3:345-358 (1991)).

The tryptophan pathway has been reported to be induced by various stress conditions including pathogen infection, oxidative stress, amino acid starvation, herbicide treatments and elicitor treatment (Zhao and Last, Plant Cell 8:2235-2244 (1996); Guyer et al., Proc. Natl. Acad. Sci. (U.S.A.) 92:4997-5000 (1995); Zhao et al., Plant Cell 10:359-370 (1998)). Accumulation of certain secondary metabolites has been reported to be coordinately regulated with expressing tryptophan pathway enzymes under inducing conditions.

2. Isoflavone Pathway

Isoflavones belong to a group of compounds called flavonoids that originate from phenylalanine and malonyl-CoA through the phenylpropanoid pathway and the flavonoid pathway. After the appropriate flavanone intermediates have been synthesized in the flavonoid pathway, isoflavones can be synthesized through two additional steps in the isoflavone pathway.

The phenylpropanoid pathway provides substrates for biosynthesis of several classes of phenolic compounds including lignins and flavonoids. For the phenylpropanoid pathway, phenylalanine ammonia-lyase (EC 4.3.1.5) is reported as both the first committed step in the pathway and a rate-limiting step. Phenylalanine ammonia-lyase catalyzes the removal of the ammonia group from phenylalanine and produces a double bond in the side chain. Phenylalanine ammonia-lyase protein has been purified from cell cultures of several species, antibodies have been produced, and the gene has been cloned in several species (Hahlbrock et al., Plant Physiol. 67:768-773 (1981); Cramer et al., EMBO J. 4:285-289 (1985); Bell et al., Mol. Cell. Biol. 5:1615-1623 (1986)).

The product of phenylalanine ammonia-lyase reaction, trans-cinnamate, is converted to 4-coumarate by cinnamate-4-hydroxylase (EC 1.14.13.11) through the introduction of a hydroxyl group into position 4 of trans-cinnamate. An isolated cinnamate-4-hydroxylase has been reported from H. tuberosus tissue and from cell cultures of Glycine max (Gabriac et al., Arch. Biochem. Biophys. 288:302-309 (1991); Kochs and Grisebach, Arch. Biochem. Biophys. 273:543-553 (1989)). A cloned cinnamate-4-hydroxylase gene has been reported from Phaseolus aureus and Arabidopsis thaliana (Mizutani et al., Biochem. Biophy. Res. Commun. 190:875-880 (1993); Bell-Lelong et al., Plant Physiol. 113:729-738, (1997)). An activation step by 4-coumarate:CoA ligase (EC 6.2.1.12) has been reported to be required before 4-coumarate can be condensed into chalcone. A cloned 4-coumarate:CoA ligase gene has been reported (Dangl, Plant Gene Res. 8:303-326 (1992)).

For the flavonoid pathway, chalcone synthase (EC 2.3.1.74) is reported as the committed and rate-limiting enzyme. Chalcone synthase provides the basic C₁₅ chalcone intermediates from which other flavonoids originate. Chalcone synthase catalyzes the condensation of three molecules of malonyl-CoA with 4-coumaroyl-CoA. Chalcone synthase reaction results in the formation of 2′,4′,6′,4-tetrahydroxychalcone. Purified chalcone synthase, antibodies that bind chalcone synthase, as well as nucleic acid sequences that encode chalcone synthase has been reported from several species (Cramer et al., EMBO J. 4:285-289 (1985); Koes et al., Plant Mol. Biol. 12:213-225 (1989)). Chalcone synthase has been reported to exist as a gene family with several genes that are differentially expressed during plant development. Chalcone synthase levels are reported to vary in response to various stress conditions.

Chalcone reductase is a NADPH-dependent polyketide reductase. The catalytic activity of chalcone reductase in combination with chalcone synthase, results in the reduction of one hydroxyl group from 2′,4′,6′,4-tetrahydrochalcone and the formation of 2′,4′,4-trihydroxychalcone (isoliquiritigenin). Chalcone reductase is required for the production of daidzin and the phytoalexins derived from it. Chalcone reductase has been reported to be induced concomitantly with chalcone synthase, after elicitor challenge, in soybean cell culture or after infection of seedlings with pathogens (Welle and Grisebach, Arch. Biochem. Biophys. 272:97-102 (1989)). Chalcone reductase protein has been purified from soybean cell culture and the isolation of a cDNA for chalcone reductase has been reported from Glycine max (Welle et al., Eur. J. Biochem. 196:423-430 (1991)).

Chalcone isomerase (EC 5.5.1.6) stereospecifically converts a chalcone into a flavanone, with the formation of an additional ring structure. Purified chalcone isomerase, antibodies that bind chalcone isomerase, as well as nucleic acid molecules that encode chalcone isomerase, have been reported from Phaseolus vulgaris and Petunia (Dixon et al., Phytochemistry 27:2801-2808 (1988)). Chalcone isomerase catalyzes the conversion of 2′,4′,6′,4-tetrahydroxychalcone and 2′,4′,4-trihydroxychalcone into naringenin and liquiritigenin, respectively. Naringenin and liquiritigenin serve as intermediates for the biosynthesis of isoflavones, anthocyanins, and other flavonoid compounds.

Isoflavanone synthase is the first reported committed enzyme for isoflavone biosynthesis. Isoflavanone synthase is a membrane-bound enzyme. Isoflavanone synthase catalyzes a cytochrome-P450-dependent oxidation in combination with a 1,2-aryl shift of the flavonoid B ring, leading to a 2-hydroxyisoflavanone intermediate. In a second reported step, isoflavanone dehydratase acts on the 2-hydroxyisoflavanone intermediate, eliminating one molecule of water. These two reactions result in the formation of genestin or daidzin, with naringenin and liquiritingenin as substrate, respectively. Both isoflavanone synthase and isoflavanone dehydratase have been partially purified from Puteraria lobata cell cultures (Hakamatsuka et al., Chem. Pharm. Bull. 37:249-258 (1989); Hashim et al., FEBS Letters 271:219-222 (1990)).

In soybean, daidzin and genestin are usually the major end products of isoflavone biosynthesis. Under pathogen attack, daidzin can be converted into phytoalexins by isoflavone reductase. Isoflavone reductase reduces isoflavones with NADPH for phytoalexin production. Isoflavone reductase is induced in response to elicitor treatment or pathogen attack. Nucleic acid sequences that encode isoflavone reductase have been reported from elicitor-challenged Medicago sativa cell cultures (Paiva et al., Plant Mol. Biol. 17:653-667 (1991)). In some species of clover, genestin and daidzin can be methylated on the 4-hydroxy group by isoflavone methyltransferase and converted into biochanin A and formononetin, respectively. The methyl donor for the reaction is S-adenosylmethionine. Methylation may also occur in the other hydroxyl positions of genestin or daidzin catalyzed by other isoflavone methyltransferases, yielding other methylated isoflavone products. Several isolated isoflavone methyltransferase enzymes with different specificity toward the hydroxyl groups of isoflavones have been reported. Only the isoflavone methyltransferase with specificity to the 4-O position can convert genestin and daidzin into biochanin A and formononetin, respectively (Khouri et al., Arch. Biochem. Biophys. 262:592-598 (1988); Edwards and Dixon, Arch. Biochem. Biophys. 287:372-379 (1991)). It has been reported the biosynthesis of isoflavones occurs in endoplasmic reticulum and the products are moved by transfer vesicles to the vacuole for storage.

3. Phenylpropanoid Pathway

Phenylpropanoid compounds are structures having a three carbon side chain on an aromatic ring derived from phenylalanine. These compounds represent a wide range of diverse phytochemicals (For reviews, see Dixon and Paiva, Plant Cell 7:1085-1097 (1995); Hahlbrock and Scheel, Annu. Rev. Plant Phys. Plant Mol. Biol. 40:347-369 (1989); Strack, In: Plant Biochemistry, Phenolic Metabolism, Dey and Harborne eds., Academic Press, pp. 387-416 (1997); Harborne, In Secondary Plant Products, Plant Phenolics, Bell and Charlwood eds., Springer-Verlag, pp. 329-402 (1988)). Phenylpropanoids are derived from cinnamic acids, which are formed from phenylalanine by the action of phenylalanine ammonia-lyase (PAL, EC 4.3.1.5) the reported branch point enzyme between the primary shikimate pathway and the secondary phenylpropanoid pathway (Chapple et al., Arabidopsis, Secondary Metabolism in Arabidopsis, Meyerowitz and Somerville eds., CSH Laboratory Press, pp. 989-1030 (1994). phenylpropanoid compounds have diverse functions due to the variations in their structures. For example, anthocyanins, as exemplified by cyanidin-3-glucoside, are low molecular weight flower pigments. Flavones, like kaempferol, are UV protectants, while glyceollin, which is a pterocarpan, act as insect repellents. Phenolic compounds, like salicyclic acid, function as signal molecules in plants. Lignins, such as coniferin, are polymeric constituents of surface and support structures and lignans, like secoisolariciresinol, are phytoestogens.

Enzymes of the phenylpropanoid pathway have been targets for herbicide screens (i.e., EPSPS and chalcone synthase (CHS)). Lignin, a phenylpropanoid compound, is important in forestry. Lignin is an undesirable component in the conversion of wood into pulp and paper. Removal of lignin is a major step in the paper making process. In addition, the digestability of herbaceous crops is affected by differences in lignin content. Phenylpropanoid pathway enzymes have also been investigated for possible nutritional applications (i.e., phytoestrogens, lignans, coumestans, and isoflavones). Reports from animals, humans, and cell culture systems have suggested that dietary phytoestrogens have an important role in the prevention of osteoporosis, cancer, and heart disease. Although there are no dietary recommendations for individual phytoestrogens, there may be a significant benefit to increase consumption (Kurzer and Xu, Annu. Rev. Nut. 17:353-381 (1997); Anderson et al., Nutrition Today 32:232-239 (1997); Kardinaal et al., Trends Food Sci. and Tech. 8:327-333, (1997)).

Erythrose-4-phosphate is biosynthesized from glucose-6-phosphate by reactions of the pentose phosphate pathway (also know as the phosphogluconate pathway). The pentose phosphate pathway has been reported to be localized in the cytosol of both photosynthetic and non-photosynthetic cells. For example, Schnarrenberger et al. (Plant Physiol. 108:609-614 (1995)) have reported that in spinach leaves the majority of the enzymatic activities of the pentose phosphate pathway were localized in the cytosol of the chloroplast. However, enzymatic activity of the pentose phosphate pathway has not been reported to be limited to photosynthetic cells. Non-photosynthetic pentose phosphate pathway enzymes have been reported to correspond to known isoforms of cytosolic pentose phosphate pathway enzymes. There are two phases of the pentose phosphate pathway, an oxidative phase resulting in the conversion of glucose-6-phosphate to ribulose-5-phosphate and a non-oxidative phase resulting in the conversion of ribulose-5-phosphate to hexose phosphate and triose phosphate (Brownleader, et al, Plant Biochemistry, In: Carbohydrate Metabolism: Primary Metabolism of Monosaccharides, Dey and Harborne eds., Academic Press, pp. 111-141 (1997); Dennis et al., Plant Metabolism, In: Glycolysis, The Pentose Phosphate Pathway and Anaerobic Respiration, Dennis et al., eds, Addison Wesley Longman Ltd., 1997, pp. 105-123 (1997)).

The first reported step in the biosynthesis of erythrose-4-phosphate is the conversion of glucose-6-phospahate to gluconolactone-6-phosphate by the NADP⁺ requiring enzyme glucose-6-phosphate dehydrogenase (EC 1.1.1.49). This reaction also generates NADPH and is reversible. Gluconolactone-6-phosphate dehydrogenase has been purified (heterotetramer of 244 kDa) from pea seedlings and has been reported to have a Kₘ for NADP⁺ of 14 μM and a Kₘ for glucose-6-phosphate of 120 μM. In E. coli, glucose-6-phosphate dehydrogenase is encoded by zfw.

Gluconolactone-6-phosphate is converted to gluconate 6-phosphate by the Mg²⁺ requiring enzyme gluconate-6-phosphate lactonase (EC 3.1.1.31). This reaction is irreversible and can also occur non-enzymatically. In E. coli, gluconate-6-phosphate lactonase is encoded by pgl.

Gluconate-6-phosphate is converted to ribulose-5-phosphate releasing CO₂ by the NADP⁺ dependent enzyme gluconate-6-phosphate dehydrogenase (EC 1.1.1.4). This reaction also generates NADPH and is irreversible. The enzyme requires divalent ions for maximum catalytic activity and is specific to NADP⁺. In E. coli, gluconate-6-phosphate dehydrogenase is encoded by gnd.

Ribulose 5-phosphate can be converted to either ribose-5-phosphate or xylulose-5-phosphate by the action of ribose-5-phosphate isomerase (EC 5.3.1.6) or ribulose-5-phosphate-3-epimerase (EC 5.1.3.1) respectively. Ribulose-5-phosphate isomerase has been reported from alfalfa shoots and spinach leaf chloroplasts and exists as dimers, trimers, or tetramers with molecular weights ranging from 40-228 kDa. The Kₘ of the ribose-5-phosphate isomerase has been reported to be between 0.5-5.0 mM. In E. coli, ribose-5-phosphate isomerase is encoded by the rpiA and rpiB genes and ribulose-5-phosphate-3-epimerase is encoded by rpe.

A transketolase (EC 2.2.1.1) requiring Mg²⁺ and thiamine pyrophosphate (TPP) catalyzes the conversion of ribulose-5-phosphate and xylulose-5-phosphate to glyceraldehyde-3-phosphate and sedoheptulose-7-phosphate. This transketolase transfers a C₂-moiety from xylulose-5-phosphate to ribose-5-phosphate yielding a C₇ keto-sugar-phosphate, (sedoheptulose-7-phosphate) and a C₃ aldo-sugar-phosphate (glycerol-3-phosphate) and is reversible. The tightly bound cofactors Mg²⁺ and TPP are required for activity. The transketolase has been purified from spinach. Both cytosolic and plastid forms of transketolase exist with the predominant activity being located in the plastid. The monomeric molecular weight of the enzyme has been reported to be 37.6 kDa and in its native form exists as a 150 kDa protein. It has a Kₘ of 100 μM for xylulose-5-phosphate and ribose-5-P. In E. coli, transketolase is encoded by tktA and tktB.

A transaldolase (EC 2.2.1.2) catalyzes the freely reversible reaction converting sedoheptulose-7-phosphate and glyceraldehyde-3-phosphate to erythrose-4-phosphate and fructose-6-phosphate. In E. coli, transaldolase is encoded by talB.

Phosphoenolpyruvate (PEP) is the precursor of pyruvate in the glycolytic pathway. The glycolytic pathway has been review in Brownleader et al., Plant Biochemistry, In: Carbohydrate Metabolism: Primary Metabolism of Monosaccharides, Dey and Harborne eds., Academic Press, pp. 111-141 (1997); Dennis et al, Plant Metabolism, In: Glycolysis, The Pentose Phosphate Pathway and Anaerobic Respiration, Dennis et al., eds., Addison Wesley Longman Ltd., pp. 105-123 (1997).

The shikamate/arogenate pathway leads to three aromatic amino acids, L-phenylalanine, L-tyrosine and L-tryptophan (Bentley, In: The Shikimate Pathway—A Metabolic Tree with Many Branches, CRC Press, 25:307-384 (1990)). These amino acids are important precursors for auxin-type plant hormones and various secondary compounds including the phenylpropanoids. There are also a number of unusual compounds that are derived from the shikimate pathway (Floss, In: Natural Product Reports 433-452, (1997)). The biosynthesis of phenylalanine which is the amino acid precursor for the biosynthesis of 4-coumaroyl-CoA, the common branch point metabolite of the phenylpropanoid pathway, involves ten reported enzyme catalyzed steps.

The first reported committed reaction in the shikimate pathway is catalyzed by the enzyme 3-deoxy-D-arabino-heptulosonate 7-phosphate synthase (DAHP synthase, EC 4.1.2.15) which controls carbon flow into the shikimate pathway. The plastid localized DAHP synthase catalyzes the formation of 3-deoxy-D-arabino-heptulosonate-7-phosphate by condensing D-erythrose-4-phosphate with phosphoenolpyruvate. DAHP synthase has been reported from plant sources including carrot and potato and has also been reported to have a substrate specificity for D-erythrose 4-phosphate and phosphoenolpyruvate. DAHP synthase has been reported to be a dimer with subunits of Mr=53,000 and is activated by Mn²⁺ (Herrmann, Plant Physiol. 107:7-12 (1995)). DAHP has not been reported to be regulated by aromatic amino acids, however, purified DAHP has been reported to be regulated tryptophan and to a lesser extent by tyrosine in a hysteric fashion (Suzich et al., Plant Physiol. 79:765-770 (1985)). In E. coli, DAHP synthase is encoded by aroF, aroG and aroH.

The next enzyme in the shikimate pathway, 3-dehydroquinate synthase (EC. 4.6.1.3), catalyzes the formation of dehydroquinate, the first carbocyclic metabolite in the biosynthesis of aromatic amino acids, from D-erythrose-4-phosphate with phosphoenolpyruvate. 3-dehydroquinate synthase reaction involves NAD cofactor dependent oxidation-reduction, β-elimination and intramolecular aldol condensation. 3-dehdroquinate synthase has been purified from Phaseolus mungo seedlings and pea seedlings and has a native molecular weight of 66,000 with a dimer subunit (Yamamoto, Phytochem. 19:779 (1980); Pompliano et al., J. Am. Chem. Soc. 111:1866 (1989)). In E. coli, 3-dehydroquinate synthase is encoded by aroB.

3-dehydroquinate dehydratase (EC 4.2.1.10) catalyzes the stereospecific syn-dehydration of dehydroquinate to dehydroshikimate and is responsible for initiating the process of aromatization by introducing the first of three double bond of the aromatic ring system. An E. coli 3-dehdroquinate dehydratase clone has been reported (Duncan, et al., Biochem. J. 238:485 (1986)). In E. coli, 3-dehydroquinate dehydratase is encoded by aroD.

Shikimate 3-dehydrogenase (EC 1.1.1.25) catalyzes the NADPH-dependent conversion of dehydroshikimate to shikimate. Bifunctional dehydroquinate dehydratase (EC 4.2.1.10) shikimate dehydrogenase has been reported in spinach, pea seedling, and corn (Bentley, Critical Rev. Biochem. Mol. Biol. 25:307-384 (1990); Kishore and Shah, Ann. Rev. Biochem. 57:67-663 (1988)). The E. coli shikimate 3-dehydrogenase has been reported to be a monomeric, monofunctional protein of molecular weight 32,000 (Chaudhuri and Coggins, Biochem. J. 226:217-223 (1985)). In E. coli shikimate 3-dehydrogenase is encoded by aroE.

Shikimate kinase (EC 2.7.1.71) catalyzes the phosphorylation of shikimate to shikimate-3-phosphate. Shikimate kinase exists in isoforms in E. coli and S. typhimurium and plant shikimate kinase has been reported from mung bean and sorghum (Bentley, Critical Rev. Biochem. Mol. Biol. 25:307-384 (1990), Kishore and Shah, Ann. Rev. Biochem. 57:67-663 (1988)). In E. coli, shikimate kinase is encoded by aroK (EC 2.7.1.71) and aroL.

5-enolpyruvyl-shikimate-3-phosphate synthase (EPSPS) (EC 2.5.1.19) catalyzes the reversible transfer of the carboxyvinyl moiety of phosphoenolpyruvate to shikimate-3-phosphate, yielding 5-enolpyruvyl-shikimate-3-phosphate. 5-enolpyruvyl-shikimate-3-phosphate synthase is the major target for inhibition by the broad spectrum, nonselective, postemergence herbicide, glyphosate. Chemical modification studies indicate that Lys, Arg, and His residues are essential for activity of the enzyme (Kishore and Shah, Ann. Rev. Biochem. 57:67-663 (1988)). 5-enolpyruvyl-shikimate-3-phosphate synthase has been isolated and characterized from microbial and plant sources including tomato, petunia, Arabidopsis, and Brassica (Kishore and Shah, Ann. Rev. Biochem. 57:67-663 (1988)). In E. coli, 5-enolpyruvyl-shikimate-3-phosphate synthase is encoded by aroA.

Chorismate synthase (EC 4.6.1.4) catalyzes the conversion of 5-enolpyruvyl-shikimate-3-phosphate to chorismate and introduces the second double bond of the aromatic ring in an trans-1,4-elimination of inorganic phosphorous. Chorismate is the last common intermediate in the biosynthesis of aromatic compounds via the shikimate pathway. Although the enzyme reaction involves no change in the oxidation state of the substrate, chorismate synthase from various sources is unusual in requiring a reduced flavin cofactor, FMNH2 or FADH2, for catalytic activity (Bentley, Critical Rev. Biochem. Mol. Biol., 25:307-384 (1990); Kishore and Shah, Ann. Rev. Biochem. 57:67-663 (1988)). In E. coli, chorismate synthase is encoded by aroC.

Tryptophan is synthesized from chorismate by the sequential action of six enzymes. The first reported step is the conversion of which begins with chorismate to anthranilate by anthranilate synthase (EC 4.1.3.27) (Radwanski and Last, Plant Cell 7:921-934 (1995)). Anthranilate is converted by phosphoribosylanthranilate synthase (EC 2.4.2.18) to 5-phosphoribosyl-anthranilate. Following this reaction phosphoribosylanthranilate isomerase catalyzes the conversion 5-phosphoribosyl-anthranilate to 1-(O-carboxyphenylamino)-1-deoxy-ribulose-5-phosphate (CdRP). Indole-3-glycerolphosphate synthase (EC 4.1.1.48) catalyzes the conversion of CdRP to indole-3-glycerolphosphate. Tryptophan synthase a (EC 4.2.1.20) catalyses the conversion indole-3-glycerolphosphate to indole. The final reported step is the conversion of indole to tryptophan by tryptophan synthase β.

Tryptophan is also the substrate for monoterpenoid indole alkaloid biosynthesis (Kutchan, Plant Cell 7:1059-1070 (1995)). Some monoterpenoid indole alkaloids of commercial importance include quinine, camptothecin, strychnine, vincristine and vinblastine. Tryptophan decarboxylase (EC 4.1.1.28) catalyzes the first reported step in monoterpenoid indole alkaloid biosynthesis, the decarboxylation of the amino acid L-tryptophan to the protoalkaloid tryptamine. A tryptophan decarboxylase clone has been reported from C. roseus (De Luca et al., Proc. Natl. Acad. Sci. (USA) 88:9969-9973 (1989)). It has homology with other aromatic L-amino acid decarboxylases from diverse plant origins. Overexpression of the tryptophan decarboxylase in tobacco (Songstad et al., Plant Physiol., 94:1410-1413 (1990); Songstad et al., Phytochemistry 30:3245-3246 (1991)) has been reported to increase production of tryptamine and tyramine (the product of L-tyrosine decarboxylation). In Brassica napus, the C. roseus tryptophan decarboxylase was reported to reduce the levels of indole glucosinolates by redirecting tyrosine pools away from indole glucosinolates (Chavadej et al., Proc. Natl. Acad. Sci. (USA) 91:2166-2170 (1994)).

Strictosidine synthase (srt1) (EC 4.3.3.2) catalyzes the first reported committed step in the biosynthesis of monterpenoid indole alkaloid. Srt1 catalyzes the stereospecific condensation of a primary amino group to tryptamine (produced via tryptophan decarboxylase) and the aldehyde moiety of the iridoid glucoside secologanin to form monoterpenoid indole alkaloid, 3α (S)-strictosidine. A strictosidine synthase cDNA clone has been has been reported from R. serpentina (Kutchan et al., FEBS Lett. 257:40-44 (1988)) and C. roseus (McKnight et al., Nucleic Acids Res. 18:4939(1990)).

Chorismate mutase (EC 5.4.99.5) catalyzes the conversion of chorismic acid to prephenic acid. Chorismic acid is a substrate for a number of enzymes involved in the biosynthesis of aromatic compounds. Plant chorismate mutase has been reported to exist as two isoforms, chorismate mutase-1 and chorismate mutase-2, that differ in feed back regulation by aromatic amino acids (Singh et al., Arch. Biochem. Biophys. 243:374-384 (1985); Goers et al., Planta 162:109-124 (1984)). It has been reported that chloroplastic chorismate mutase-1 plays a role in biosynthesis of aromatic amino acids as this enzyme is activated by tyrosine and phenylalanine. The cytosolic isozyeme chorismate mutase-2 is not regulated by aromatic amino acids and has been reported to play a role in providing the aromatic nucleus for synthesis of aromatic secondary metabolites including tocopherol (d'Amato et al., Planta 162:104-108 (1984)). In E. coli, chorismate mutase is encoded by pheA.

Prephenate dehydratase (EC 4.2.1.51, EC 5.4.99.5), in E. coli, has been reported to be a bifunctional protein with chorismate mutase (EC 5.4.99.5). In E. coli the chorismate mutase-prephenate dehydratase multi-functional protein catalyzes the conversion from chorismate to phenylpyruvate and is encoded pheA. The multifunctional chorismate mutase-prephenate dehydratase in E. coli has been reported to have a Km of 45 μM.

Prephenate dehydrogenase (EC 1.3.1.12, EC 5.4.99.5), in E. coli, is also reported to be a bifunctional protein with chorismate mutase (EC 5.4.99.5). In E. coli the chorismate mutase-prephenate dehydrogenase multi-functional protein catalyzes the conversion from chorismate to 4-hydroxyphenylpyruvate and is encoded tyrA. The multifunctional chorismate mutase-prephenate dehydrogenase in E. coli has been reported to have a Km of 92 μM and 50 μM for prephenate.

Tyrosine aminotransferase (EC 2.6.1.5), in E. coli, is encoded by tyrB. Tyrosine aminotransferase catalyzes the last reported step in phenylalanine and tyrosine biosynthesis by transferring an amino group from glutamate to either phenylpyruvate or 4-hydroxyphenylpyruvate, releasing 2-ketoglutarate, and generating phenylalanine and tyrosine respectively. There are other amino transferases in E. coli have been reported to catalyze the reaction, for example, aspartate amino transferase (aspC) (EC 2.6.1.1) or the branched chain amino acid aminotransferase (ilvE) (EC 2.6.1.42)

Prephenate aminotransferase catalyzes the conversion of prephenate to L-arogenate by transferring an amino group from glutamate to prephenate and releasing 2-ketoglutarate. Prephenate aminotransferase is dependent on pyridoxial 5′-phosphate and glutamate.

Arogenate dehydratase (EC 4.2.1.91) catalyzes the conversion of L-arogenate to tyrosine. Arogenate dehydratase is inhibited by phenylalanine. Arogenate dehydratase is utilized by plants and certain microbes for the production of tyrosine.

Arogenate dehydrogenase (EC 1.3.1.43) catalyzes the conversion of L-arogenate to phenylalanine. Arogenate dehydrogenase is inhibited by tyrosine. Arogenate dehydrogenase is utilized by plants and certain microbes for the production of phenylalanine.

Phenylalanine ammonium lyase (PAL) (EC 4.3.1.5) catalyzes the reported committed step in phenylpropanoid biosynthesis. In certain grasses and fungi, PAL may also act on tyrosine to produce 4-coumarate directly. PAL is a tetrameric enzyme of native MW 270,000 to 330,000 Kd and a pH optimum of 8-9. Phenylalanine analogues such as L-2-amino-oxy-3-phenylpropionic acid (L-AOPP) or 2-amino-indan-2-phosphonic acid (A1P) inhibit PAL activity in nanomole concentrations. PAL has been reported to be localized in the microsomal compartment suggesting that it is associated with the endoplasmic reticulum. In certain grasses and fungi, PAL, or another enzyme designated TAL (tyrosine ammonium lyase), may also act on tyrosine leading directly to 4-coumarate.

Cinnamate-4-hydroxylase (C4H) (EC 1.14.13.11) is a cytochrome P450-linked monooxygenase (molecular oxygen is cleaved during this reaction, one oxygen transferred to the aromatic ring the other to water). Cinnamate-4-hydroxylase catalyzes the hydroxylation of cinnamic acid to 4-coumarate. C4H clones have been reported (Fahrendorf and Dixon, Arch. Biochem. Biophys. 305:509-515 (1993); Mizutani et al., Biochem. Biophys. Res. Comm. 190:875-880 (1993); Teutsch et al., Proc. Natl. Acad. Sci. (USA), 90:4102-4106(1993)) from several different plant species and functionally expressed in yeast (Fahrendorf and Dixon, Arch. Biochem. Biophys. 305:509-515 (1993); Pierrel et al., Eur. J. Biochem. 224:835-844 (1994)). It has been reported that metabolic channeling of substrates between PAL and C4H exist (Hrazdina and Jensen, Annu. Rev. Plant. Physiol. Plant Mol. Biol. 43:241-267 (1992)).

Hydroxycinnamate:CoA ligase (also known as 4-coumarate:CoA ligase or AMP forming) (4CL) (EC 6.2.1.12) catalyzes the formation of CoA thioesters of cinnamic acids in the biosynthesis of a wide variety of phenolic derivatives. 4CL is an ATP dependent enzyme, and different isoforms with different substrate specificities can exist. Most reported 4CL enzymes have broad specificity. 4CL displays low reported activity with sinapic acid. 4CL clones have been reported from several plant species, including Arabidopsis (Lee et al., Plant Mol. Biol. 28:871-884 (1995)). Anti-sensed experiments of PAL transcription in Arabidopsis have been reported to reduce lignin content (Lee et al., Plant Cell 9:1985-1998 (1997)).

Lignins are polymers of aromatic subunits that are usually derived from phenylalanine. Lignins serve as a matrix around the polysaccharide components of some plant cell walls providing strength and water impermeability and defense against pathogens. Lignin is one of the world's most abundant polymers along with cellulose and chitin. Lignin monomer (monolignol) biosynthesis occurs in the cytosol of plants and precursors to lignin formation are typically stored in vacuoles (Whetten and Sederoff, Plant Cell 7:1001-1013 (1995); Campbell and Sederoff, Plant Physiol. 110:3-13 (1996)).

4-hydroxycinnamate 3-hydroxylase (C3H) catalyzes the hydroxylation of 4-coumarate to form caffeate. Several plant oxidases can carry out the hydroxylation of phenolic molecules. It has been reported that the reaction is catalyzed by a phenolase (EC 1.10.3.1), however inhibitor studies of the phenolase have not been reported to cause a decrease on caffeic acid synthesis in mung bean seedlings (Duke and Vaughn, Physiol. Plant 54:381-385 (1982)).

S-adenosylmethionine:caffeate/5-hydroxylase-O-methylransferase (C-OMT) (EC 2.1.1.68) catalyzes the methylation of caffeic acid to produce ferulic acid using S-adenosyl methionine as the methyl group donor. C-OMT has also been reported to catalyze the methylation of 5-hydroxyferulate to form sinapate. C-OMT clones have been reported from several plant species. C-OMT role in monolignin biosynthesis has been confirmed in both monocots and dicots. There have been reports of different substrate specificities of C-OMT (from crude extracts) isolated from gymnosperms with a reported substrate preference for caffeate and angiosperms with a reported substrate preference for 5-hydroxyferulate with respect to both caffeic acid and 5-hydroxyferulate methylation.

Ferulate 5-hydroxylase (F5H) has been reported to be catalyzed by a cytochrome P-450-linked monooxygenase in the conversion of ferulate to 5-hydroxyferulate. An Arabidopsis mutant (fah-1) has been reported (Shapple et al., Plant Cell 4:1413-1424 (1992)). F5H has been reported to also be associated with the regulation of lignin content in angiosperms and gymnosperms.

4-hydroxycinnamoyl-CoA 3-hydroxylase (CCoA-3H) is an alternative enzyme to the hydroxylation of free 4-coumarate by directly hydroxylating 4-coumaroyl-CoA to caffeoyl-CoA. CCoA-3H has been reported to be a FAD (and possibly NADPH dependent) enzyme in lignin biosynthesis (Kamsteeg et al., Pflanzenphysiol. 102:435-442 (1981); Boniwell and Butt, Z. Naturforsch 41C:56-60 (1986)).

S-adenosylmethionine:caffeoyl-CoA/5-hydroxyferulate-CoA-O-methyltransferase (CCoA-OMT) (EC 2.1.1.104) is another methylating enzyme distinct from C-OMT. CCoA-OMT has been reported to play a role in the methylation of both caffeoyl-CoA and 5-hydroxyferuloyl-CoA during monolignol biosynthesis. Several CCoA-OMT cDNA clones have been reported (Schmitt et al., J. Biol. Chem. 266:17416-17423 (1991); Ye et al., Plant Cell 6:1427-1439 (1994)).

Hydroxycinnamoyl-CoA:NADPH oxidoreductase (CCR) (EC 1.2.1.44) catalyzes the reduction of the hydroxycinnamoyl-CoA thioesters to their corresponding aldehydes. The enzyme has been reported to be generally non-specific for hydroxycinnamoyl-CoA thioesters, although in some plant species CCR has a reported preference for feruloyl-CoA. CCR may play a key regulatory role as the first reported committed step in the biosynthesis of monolignols from the phenylpropanoids (Goffner et al., Plant Physiol. 106:625-632 (1994)).

Hydroxycinnamyl alcohol dehydrogenase (CAD) (EC 1.1.1.195) catalyzes the reduction of hydroxycinnamaldehydes to hydroxycinnamyl alcohols. CAD is regulated by both developmental and environmental factors, much like other well studied enzymes of the phenylpropanoid pathways. CAD can exist as a single gene or as multiple isoforms. CAD from gymnosperms have been reported to be more active on coniferaldehyde, whereas angiosperm CAD have equal activities on either coniferaldehyde or sinapaldehyde.

Conifer alcohol dehydrogenase (EC 1.1.1.194) catalyzes the reduction of coniferaldehyde to coniferyl alcohol (Mansell et al., Phytochemistry 37:683-688 (1976); Wyrambik and Grisebach, Eur. J. Biochem. 59:9-15 (1976)).

UDP-Glc:coniferyl alcohol 4-O-glucosyltransferase (EC 2.4.1.111) catalyzes the glycosylation on the phenolic hydroxy group to form the monolignol glucosides (4-hydroxycinnamyl alcohol glucoside, coniferin and syringin (i.e., UDP glucose plus conifer alcohol yields coniferin plus glucose). These glucosides accumulate in some species of plants (i.e., conifers) and have been reported to be localized in the vacuole. Monolignol compounds are relatively toxic and unstable and do not accumulate to high levels, therefore the glycosylation of the monolignols renders them nontoxic and stabilizes the molecule. Monolignol glucosides have been reported to be primary candidates for transport across cell membranes. The glucosyltransferases have been purified from several species of trees, but the gene has not yet been identified.

Coniferin-β-glucosidase (EC 3.2.1.126; EC 3.2.1.21) is involved in the hydrolysis of the monolignol glucosides to the corresponding alcohols, releasing glucose in the process (i.e., coniferin plus water yields conifer alcohol plus glucose). Coniferin-β-glucosidase has been reported to be localized in the cell walls, the reported site of lignin biosynthesis.

Peroxidase (EC 1.11.1.7), a H₂O₂-dependent hemoprotein and an oxygen-dependent oxidase containing four copper atoms, laccase (EC 1.10.3.2), oxidize monolignols in vitro to their respective free radicals for initiation of the polymerization reaction. Reviews by O'Malley et al., Plant J 4:751-757 (1993); Dean and Eriksson, Holzforschung 48:21-33 (1994); Liu et al., Plant J 6:213-224 (1994); McDougall et al., Phytochemistry 37:683-688 (1994), have reported various roles for peroxidases and laccases in lignin polymerization.

Lignans are a widely distributes class of natural products. Reported functions of lignans in plants are mainly involved in pathogen defense mechanisms. Until recently the biochemical pathways leading to lignan formation, more specifically to secoisolaricresinol, was unknown. There are three enzymes involved in the biosynthesis of secoisolariciresinol in Forsythia intermedia (laccase, dirigent protein, and pinoresinol/laricresinol reductase). There are several classes of lignans, neolignans and related compounds (Ward, Natural Product Reports 43-74 (1997). A role for lignans as a form of cancer chemotherapy and prevention have been suggested. The plant lignans, secoisolariciresinol and matairesinol are precursors to the mammalian lignans, enterodiol and enterolactone which are formed through bioconversion of the plant lignans by microbes colonizing the gastrointestinal tract.

The dirigent protein is a protein that has no detectable enzymatic activity (Davin et al., Science 275:362-366 (1997)). Dirigent protein reported physiological role is to bind and orient free radicals generated in conifer alcohol by the laccase to allow for stereoselective bimolecular phenoxyradical coupling to occur to generate (+) pinoresinol in Forsythia. The dirigent protein has a native molecular weight of about 78 kDa (about 27 kDa subunit MW suggesting that the protein is a trimer) and appears to be glycosylated.

Laccase (EC 1.10.3.2) or oxidase (EC 1.11.1.7) has been reported to produce the free radical in the conifer alcohol required for phenoxyradical coupling (Davin et al., Science 275:362-366 (1997)). Laccase (native Mw 120 kDa) has been reported from several different plant species involved in lignification. Laccase is involved in lignification and works with the dirigent protein to form (+)-pinoresinol in Forsythia. In the absence of a dirigent protein, the reaction proceeded by the radical formation generates a racemic mixture of (+/−)-pinoresinol.

Pinoresinol/lariciresinol reductase has been reported to exist as two isofunctional forms in Forsythia intermedia (Dinkova-Kostova et al., J. Biol. Chem. 271:29473-29482 (1996)). Both reported isoforms catalyze the sequential reduction of (+)-pinoresinol to (+)-lariciresinol to (−)-secoisolariciresinol and have similar kinetic properties. Pinoresinol/lariciresinol reductase has a reported monomeric molecular weight of 36 kDa. Pinoresinol/lariciresinol reductase has been expressed in E. coli.

Secoisolariciresinol dehydrogenase has been reported to be a 57 kDa NADP dependent enzyme that produces (−)-matairesinol from the precursor molecule (−)-secoisolariciresinol in Forsythia. Matairesinol is metabolized to the mammalian lignan enterolactone by gastrointestinal flora (i.e., Clostridia sp.) or human fecal flora. This conversion can be through a number of independent pathways requiring a reduction step, a dehydration step, a demethylation step and finally a racemization step.

Secoisolariciresinol glucosyltransferase catalyzes the conversion of secoisolariciresinol to secoisolariciresinol diglucoside (SDG). The point of attachment of the glucose residues is C₉/C₉ rather than to the phenolic group.

Flavonoids are a large class of compounds ubiquitous in plants (usually occurring as glycosides). Flavonoids contain several phenolic hydroxyl functions attached to ring structures designated A, B, and C. Flavonoids are classified according to the oxidation state of the heterocyclic ring C (pyran ring) which connects the two benzene rings A and B. Structural variations within the ring structure subdivide the flavonoids into several classes. Flavonols (with the 3-OH pyran-4-one ring), flavones (lacking the 30H group), flavanols (lacking the 2,3 double bond and the 4-one structure), and isoflavones (B ring is located in the 3 position on the C ring). The flavone, naringenin, is the precursor to three different flavonoid classes (flavones, isoflavones, and dihydroflavonols). The precursors for the synthesis of all reported flavonoids are malonyl-CoA (derived from the carboxylation of acetyl-CoA using the enzyme acetyl-CoA carboxylase (EC 6.4.1.2)) and 4-coumaroyl-CoA. Flavanoid biosynthesis and flavanoid compounds perform a wide range of functions ranging from pigment, UV protectors, antioxidants, and phytoestrogens (Dixon and Paiva, Plant Cell 7:1085-1097 (1995); Rice-Evans et al., Trends in Plant Sciences 2:152-159, (1997); Kardinaal et al., Trends in Food Sci. and Tech. 8:327-333 (1997); Anderson and Garner, Nutrition Today 32:232-239 (1997); Kurzer and Xu, Annu Rev. Nutr. 17:353-381 (1997); Holton and Cornish, Plant Cell 7:1071-1083 (1995)).

Chalcone synthase (CHS) (EC 2.3.1.74) (also know as malonyl-CoA:4-coumaroyl-CoA malonyltransferase) catalyzes the stepwise condensation of three acetate units from malonyl-CoA with 4-coumaroyl-CoA to form chalcone (4,2′, 4′, 6′-tetrahydroxychalcone). CHS as been reported to be the rate limiting enzyme in flavonoid synthesis, channeling hydroxycinnamates into flavonoid biosynthesis. Chalcone synthase has been reported to be a dimeric protein (two identical subunits) with a Mw of 78 kDa to 88 kDa. Genes encoding CHS have been cloned from several different species of plants and in some cases is part of a huge multigene family. CHS is highly specific for 4-coumaroyl-CoA but others are accepted in the reaction. Another enzyme that is reported to be closely related to chalcone synthase is stilbene synthase. Stilbene synthase or resveratrol synthase catalyzes through a different folding mechanism the formation of resveratrol a compound implicated to be beneficial for human health.

Chalcone isomerase CHI) (EC 5.5.1.6) catalyzes the isomerization (ring closure) of chalcone to naringenin (5, 7, 4′-trihydroxyflavanone). Naringenin is considered the progenitor of essentially all other flavonoid structures. Chalcone isomerase has been cloned from a number of different plant species. Isomerization of chalcone to form naringenin can occur spontaneously, but at a much slower rate. Plants devoid of chalcone isomerase activity accumulate chalcone, and produce yellow pigments.

Chalcone reductase (CHR) catalyzes the reduction of chalcone to form 4, 2′, 4′-trihydroxychalcone an important precursor to the isoflavone daidzein. Isoflavone synthase (IFS) (also known as 2-hydroxyisoflavone synthase) is an NADPH: oxygen oxidoreductase with a dehydratase reaction. Isoflavone synthase catalyzes the oxidative 2,3-aryl shift of naringenin or 7,4′-dihydroxyflavanone to yield genistein or daidzein respectively. The initiating step in isoflavone can be an epoxidation that is catalyzed by a cytochrome P450-dependent monooxygenase. After the structural rearrangement, aryl shift, and addition of a C2 hydroxyl group, elimination of H₂O by a dehydratase yields the isoflavone structure. The dehydratase is most likely a separate enzyme. In alfalfa for example, the isoflavones daidzein and genistein can be methylated at the 4′ position by a 4′ methyltransferase to generate formononetin and biochanin A respectively. In soybean for example the isoflavones are glycosylated at the 7 position and are further malonylated or acetylated to yield the malonyl and acetyl glycones. The isoflavones daidzein and genistein are metabolized by gastrointestinal flora to yield equol and p-ethylphenol respectively.

Flavone synthase I (FLSI) (also known as 2-hydroxyflavanone synthase, flavanone 2-oxoglutarate:oxidoreductase) and flavone synthase II (FLSII) catalyze the conversion of naringenin to flavone.

Flavonol synthase I (dioxygenase) (also known as dihydroflavonol and 2-oxoglutarate-L-oxidoreductase) catalyzes the formation of kaempferol from dihydrokaempferol.

Flavanone 3-hydroxylase (F30H) (EC 1.14.11.9) catalyzes the hydroxylation of the 3 position of naringenin to yield dihydrokaempferol. Flavanone 3-hydroxylase clones from snapdragon (Martin et al., Plant J. 1:3749 (1991)) and petunia petals (Britsch et al., J. Biol. Chem. 267:5380-5387 (1992)), have been reported.

Another reported function of the flavonoid pathway involves the biosynthesis of pigment dyes known as anthocyanins. Anthocyanin biosynthesis in petals is involved in attracting pollinators or in fruits for seed dispersal. Anthocyanins are also protectors against UV irradiation or feeding related damages (Holton and Cornish, Plant Cell 7:1071-1083 (1995)).

Flavanoid 3′-hydroxylase (F3′OH) (EC 1.14.13.21) catalyzes the 3′ hydroxylation of naringenin or dihydrokaempferol.

Flavanoid 3′,5′-hydroxylase (F3′5′OH) (EC 1.14.13.21) catalyzes the 3′ and 5′ hydroxylation of dihydrokaempferol. A flavanoid 3′,5′-hydroxylase clone has been reported from petunia (Holton et al., Nature 366:276-279 (1993)).

Dihydroflavonol 4-reductase (DFR) catalyzes the reduction of the colorless dihydroflavonols (dihydrokaempferol, dihydroquercetin, and dihydromyricetin) to produce the flavan-3,4-cis diols (leucoanthocyanidins). Further oxidation and dehydration of the different leucoanthocyanidins (i.e., leucopelargonidin) is catalyzed by anthocyandin synthase (ANS). Glycosylation of the 3 position by anthocyanin glycosyltransferase (3GT) yields the corresponding brick red pelargonidin, red cyanidin, and blue delphinidin pigments. Depending on the plant species, 3-glucosides can undergo further methylation or acylation or glycosylation by anthocyanin methyltransferase.

Isoflavone daidzein is the precursor to pterocarpans (infection induced phytoalexins) involved in plant defense responses. One example of pterocatpan biosynthesis is glyceollinsins from Glycine max (soybean). This seven enzyme pathway results in the formation of glyceollin I, II, and III.

Isoflavone 2′-hydroxylase (also known as isoflavone NADPH:oxygen oxidoreductase) and 2′-hydroxyisoflavone reductase (also know as 2′-hydroxyisoflavone:NADPH oxidoreductase), pterocarpan synthase (also known as 2′-hydroxyisoflavone:NADPH oxidoreductase), and pterocarpan 6α-hydroxylase (also known as pterocarpan:NADPH oxidoreductase) catalyze the conversion of daidzein to glycinol. The oxygenases involved are membrane bound cytochrome P450 monooxygenases.

Prenyltransferase I (DMAPP) (also known as dimethylallylpyrophosphate), glycinol 2-dimethylallyltransferase and pterocarpan cyclase (also known as dimethylallylglycinol NADPH:oxidoreductase) catalyze the conversion of glycinol to glyceollin I. Both prenyltransferases are Mn²⁺ dependent and catalyzes the transfer of a dimethylallyl moiety from DMAPP to C2 or C4 of glycinol. Pterocarpan cyclase catalyzes the cyclization of both 2 and 4 dimethylallylglycinols and appears to be a P-450-monooxygenase.

Prenyltransferase II (also known as DMAPP:glycinol 4-dimethylallyltransferase) and pterocarpan cyclase (also known as dimethylallylglycinol NADPH:oxidoreductase) catalyze the conversion of glycinol to either glyceollin II or glyceollin III.

4. Isoprenoid Metabolism

i. Carotenoid Pathway

Carotenoids are synthesized in higher plants via a portion of the isoprenoid pathway that starts with the formation of phytoene from geranylgeranyl pyrophosphate (GGPP). This is the first reported committed step for carotenoid biosynthesis and is part of a larger isoprenoid pathway. A basic isoprenoid precursor for the entire pathway, isopentenyl pyrophosphate (IPP), may be formed via two pathways, the mevalonate and non-mevalonate pathway. The non-mevalonate pathway predominates in plastids where carotenoid, tocopherols, xanthophylls, etc., are synthesized. This pathway has been studied in algae and higher plants (Schwender et al., Biochem. J. 316:73-80 (1996), Arigoni et al., Proc. Natl. Acad. Sci. (U.S.A.) 94:10600-10605 (1997)). In the non-mevalonate pathway, isopentenyl pyrophosphate is formed from pyruvate and glyceraldehyde-3-phopsphate via a multi-step enzyme reaction. In the first step in this pathway, 1-deoxyxyulose-5-phosphate synthase forms 1-deoxyxylulose-5-phosphate.

Isopentenyl pyrophosphate isomerase catalyzes the reversible isomerization between IPP and dimethylallyl pyrophosphate (DMAPP). This enzyme has been cloned from plant sources (Blanc and Pichersky, Plant Physiol. 108:855-856 (1995)). Utilizing a pool of IPP and DMAPP, geranylgeranyl pyrophosphate synthase (GGPPS) catalyzes the formation of GGPP(C20). GGPPS has been cloned from peppers (Kuntz et al., Plant J. 2:25-34 (1992)) where it is reported to increase in activity and transcript level during the ripening and carotenoid deposition phase of pepper fruit. It has also been cloned from other plants including Arabidopsis thaliana (Zhu et al., Plant Cell Physiol. 38:357-361 (1997)) and lupin (Aitken et al., Plant Physiol. 108:837-838 (1995)).

GGPP is reported to be a precursor to carotenoid, tocopherols and chlorophyll in the plastids. Phytoene synthase catalyses the formation of phytoene from two GGPP molecules. This step is the first reported committed step in carotenoid biosynthesis. Phytoene synthase (EC 2.5.1.32) has been cloned from a number of plants including tomato (Fray et al., Plant Mol. Biol. 22:589-602 (1993)), melon (Karvouni et al., Plant Mol. Biol. 27:1153-1162 (1995)) and maize (Buckner et al., Genetics 143:479-488 (1996)). Phytoene has 9 double bonds.

The next reported major carotenoid in the pathway, lycopene, has 13 double bonds. In bacteria, one enzyme, phytoene desaturase (EC1.3.99.-), performs all 4 desaturations. In plants, phytoene desaturase adds two double bonds generating zeta-carotene.

Phytoene desaturase has been cloned from tomato and other plants species and is reported to be developmentally regulated by carotenoid accumulation (Pecker et al., Proc. Natl. Acad. Sci. (U.S.A.) 89:4962-4966 (1992); Li et al., Plant Mol. Biol. 30:269-279 (1996); Hugueney et al., Eur. J. Biochem. 209:399-407 (1992)). A second enzyme, zeta-carotene desaturase, adds two additional double bonds yielding lycopene, has been cloned from pepper (Klein et al., FEBS Lett. 372:199-202 (1995)). Once lycopene has been formed, lycopene beta cyclase catalyzes the formation of 6 membered rings at either end of lycopene. Beta rings are formed via lycopene beta-cyclase to yield beta-carotene (two rings) or gamma-carotene (one ring). Lycopene beta-cyclase has been cloned from Arabidopsis thaliana (Cunningham et al., The Plant Cell 8:1613-1626 (1996)) and tomato (Pecker et al., Plant Mol. Biology 30:807-819 (1996)). Lycopene epsilon-cyclase catalyzes a reaction that adds an epsilon ring to either lycopene or neurosporene. The addition of a beta ring and an epsilon ring to lycopene generates alpha-carotene. In addition, lycopene beta-cyclase can cyclize an end of neurosporene. Lycopene epsilon cyclase has also been cloned from Arabidopsis thaliana (Cunningham et al., The Plant Cell 8:1613-1626 (1996)).

Xanthophylls are a class of carotenoids having oxygen-containing groups. The cyclohexene rings of both alpha and beta-carotene can be further modified by the action of different enzymes. One such class of enzymes, generically named carotene hydroxylases, introduces hydroxyl groups into specific positions in the cyclohexene rings of the carotene molecule. The resulting products are hydroxyl xanthophylls, examples of which include zeaxanthin and cryptoxanthin. A cloned beta-carotene hydroxylase gene from plants is reported in Sun et al., J. Biol. Chem. 271:24349-24352 (1996). Beta-carotene hydroxylase catalyses the addition of hydroxyl groups to beta rings of beta-carotene.

Several compounds result from the hydroxylation of beta-carotene. For example, beta-cryptoxanthin is formed by hydroxylation of beta-carotene. Hydroxylation of beta-cryptoxanthin results in zeaxanthin (two hydroxylations). When a hydroxyl group is added to both the beta and epsilon rings of alpha-carotene, a xanthophyll lutein is formed. An Arabidopsis thaliana epsilon hydroxylase mutant that adds a hydroxyl group to an epsilon ring has been reported (Pogson et al., Plant Cell 8:1627-1639 (1996)).

Another class of enzymes that acts on carotene cyclohexene rings is ketolases. These enzymes introduce keto groups at specific positions of the carotene cyclohexene rings. Genes encoding some of these ketolase enzymes have been reported in marine bacteria and algae. One such enzyme, beta-carotene ketolase, produces echinenone from beta-carotene with the addition of a keto group and further oxidation of echinenone produces canthaxanthin (two keto groups added). Ketolase genes have been cloned from marine bacteria (Misawa et al., J. Bacteriol. 177:6575-6584 (1995)), and from the green algae, Haematococcus pluvialis (Lotan and Hirschberg, FEBS Lett. 364:125-128 (1995)).

Certain hydroxylase and ketolase enzymes have been reported to act asymmetrically on one of the cyclohexene rings of the carotene molecule resulting in accumulation of specific classes of xanthophylls with reduced oxidation, such as echinenone (Fernandez-Gonzalez et al., J. Biol. Chem. 272:9728-9733 (1997)). Hydroxylases and ketolases can act simultaneously on the same substrate carotene molecule to generate different compounds that contain both hydroxy and keto groups. Examples of such mixed hydroxy and keto xanthophylls include compounds such as hydroxyechinenone, phoenicoxanthin adonixanthin and astaxanthin. Although astaxanthin is not typically found in plants, it is found in the petals of Adonis aestivalis.

In photosynthesis the violaxanthin cycle inter-converts xanthophylls zeaxanthin, antheraxanthin and violaxanthin. The violaxanthin cycle is reported to modulate excess light energy that can damage the photosynthetic apparatus. Two genes are reported to be associated with the violaxanthin cycle. Zeaxanthin epoxidase converts zeaxanthin to antheraxanthin and antheraxanthin to violaxanthin. Violaxanthin deepoxidase converts violaxanthin to antheraxanthin and antheraxanthin to zeaxanthin. Genes for both of these enzymes have been isolated from plants (Bugos et al., Proc. Natl. Acad. Sci. (U.S.A.) 93:6320-5325 (1996), Marin et al., EMBO J. 15:2331-2342 (1996)). Abscisic acid can be formed from carotenoid. An abscisic acid biosynthesis mutant of Arabidopsis thaliana was reported to correspond to the zeaxanthin epoxidase gene.

Antheraxanthin and violaxanthin can be converted into the ketocarotenoid capsanthin and capsorubin, respectively. Capsanthin and capsorubin are reported to be responsible for imparting the characteristic bright red color to peppers. The gene encoding this enzyme has been isolated from peppers (Bouvier et al., Plant J. 6:45-54 (1994)).

In plants and bacteria xanthophylls can be esterified to sugar moieties. A gene for an enzyme capable of esterifying zeaxanthin has been cloned from Erwinia (Misawa et al., J. Bacti. 172:6704-6712 (1990)). Carotenoids in green tissues are reported to be found in the plastids associated with cellular membranes and also sequestered into discrete structures in tissues, such as petals or fruits, that accumulate large amounts of carotenoid. These discrete structures are composed of carotenoid, lipids and proteins termed carotenoid binding proteins. Carotenoid binding proteins have been isolated from peppers, and have been reported to aid in the sequestration of carotenoid, (Deruere et al., Plant Cell 6:119-133 (1994)) and cucumbers (Vishnevetsky et al., Plant Journal 10:1111-1118 (1996)).

Zeta-carotene desaturase catalyzes the conversion of zeta-carotene to lycopene. It has been reported that there is a relationship between zeta-carotene desaturase and phytoene desaturase from bacteria and fungi (Misawa et al., Plant Mol. Biol. 24:369-379 (1994)). Zeta-carotene desaturase has been isolated in Escherichia coli (Albrecht et al., Eur. J. Biochem. 236:115-120 (1996)).

ii. Tocopherol Synthesis Pathway

The chloroplast of higher plants exhibit interconnected biochemical pathways that lead to secondary metabolites, including tocopherols, that not only perform functions in plants but can also be important for mammalian nutrition. In plastids, tocopherols account up to 40% of the total quinone pool. The biosynthetic pathway of α-tocopherol in higher plants involves condensation of homogentisic acid and phytylpyrophosphate to form 2-methyl-6 phytylbenzoquinol (Fiedler et al., Planta 155:511-515 (1982); Soll et al., Arch. Biochem. Biophys. 204:544-550 (1980); Marshall et al., Phytochem. 24:1705-1711 (1985)). The plant tocopherol biosynthetic pathway can be divided into four parts: synthesis of homogentisic acid, which contributes to the aromatic ring of tocopherol; synthesis of phytylpyrophosphate, which contributes to the side chain of tocopherol; cyclization which plays a role in chirality and chromanol substructure of the vitamin E family; and S-adenosyl methionine dependent methylation of an aromatic ring, which effects the compositional quality of the vitamin E family.

Homogentisate is an aromatic precursor in the biosynthesis of tocopherols in chloroplasts and is formed from the aromatic shikimate metabolite, p-hydroxyphenylpyruvate. The aromatic amino acids phenylalanine, tyrosine, and tryptophan are formed by a reaction sequence that initiates from the two carbohydrate precursors, D-erythrose 4-phosphate and phosphoenolpyruvate, via shikimate, and forms prearomatic and aromatic compounds (Bentley, Critical Rev. Biochem. Mol. Biol. 25:307-384 (1990)). Approximately 20% of the total carbon fixed by green plants is routed through the shikimate pathway with end products being aromatic amino acids and other aromatic secondary metabolites such as flavonoids, vitamins, lignins, alkaloids, and phenolics (Herrmann, Plant Physiol. 107:7-12 (1995), Kishore and Shah, Ann. Rev. Biochem. 57:67-663 (1988)). Various aspects of the shikimate pathway have been reviewed (Bentley, Critical Rev. Biochem. Mol. Biol. 25:307-384 (1990); Herrmann, Plant Physiol. 107:7-12 (1995); Kishore and Shah, Ann. Rev. Biochem. 57:67-663 (1988)).

The first reported committed reaction in the shikimate pathway is catalyzed by the enzyme 3-deoxyarabino-heptulosonate 7-phosphate synthase (also known as 3-deoxy-D-arabino-heptulosonate 7-phosphate synthase, deoxyarabino-heptulosonate-P-synthase, and DAHP synthase (EC. 4.1.2.15)), which has been reported to control carbon flow into the shikimate pathway. The plastid localized DAHP synthase catalyzes the formation of 3-deoxy-D-arabino-heptulosonate 7-phosphate by condensing D-erythrose 4-phosphate with phosphoenolpyruvate. DAHP synthase has been isolated from plant sources including carrot and potato. DAHP synthase has substrate specificity for D-erythrose 4-phosphate and phosphoenolpyruvate, is a dimer of subunits having a molecular weight of 53 KD and is activated by Mn²⁺ (Herrmann, Plant Physiol. 107:7-12 (1995)). Aromatic amino acids are not reported to act as feedback regulators. Purified DAHP synthase is activated by tryptophan and, to a lesser extent, by tyrosine in a hysteric fashion (Suzich et al., Plant Physiol. 79:765-770 (1985)).

The next reported enzyme in the shikimate pathway is 3-dehydroquinate synthase (EC 4.6.1.3), which catalyzes the formation of dehydroquinate, the first carbocyclic metabolite in the biosynthesis of aromatic amino acids, from the substrates D-erythrose 4-phosphate and phosphoenolpyruvate. The enzyme reaction involves a NAD (nicotinamide adenine dinucleotide) cofactor dependent oxidation-reduction, β-elimination, and an intramolecular aldol condensation. 3-Dehydroquinate synthase has been purified from Phaseolus mungo seedlings and pea seedlings, has a native molecular weight of 66 KD and is a dimer (Yamamoto, Phytochem. 19:779-802 (1980); Pompliano et al., J. Am. Chem. Soc. 111:1866-1871-1871 (1989)).

3-Dehydroquinate dehydratase (EC 4.2.1.10) catalyzes the stereospecific syn-dehydration of dehydroquinate to dehydroshikimate and has been reported to be responsible for initiating the process of aromatization by introducing the first of three double bonds of the aromatic ring system. 3-Dehydroquinate dehydratase has been cloned from E. coli (Duncan et al., Biochem. J. 238:475-483 (1986)).

Shikimate dehydrogenase (EC 1.1.1.25) catalyzes the NADPH (reduced nicotinamide adenine dinucleotide phosphate)-dependent conversion of dehydroshikimate to shikimate. Bifunctional 3-dehydroquinate dehydratase-shikimate dehydrogenase has been reported in spinach, pea seedling, and maize (Bentley, Critical Rev. Biochem. Mol. Biol. 25:307-384 (1990), Kishore and Shah, Ann. Rev. Biochem. 57:67-663 (1988)). E. coli shikimate dehydrogenase has been reported to be a monomeric, monofunctional protein with a molecular weight of 32,000 daltons (Chaudhuri and Coggins, Biochem. J. 226:217-223 (1985)).

Shikimate kinase (EC 2.7.1.71) catalyzes the phosphorylation of shikimate to shikimate-3-phosphate. Shikimate kinase exists as isoforms in E. coli and S. typhimurium. Plant shikimate kinase has been partially purified from mung bean and sorghum (Bentley, Critical Rev. Biochem. Mol. Biol. 25:307-384 (1990); Kishore and Shah, Ann. Rev. Biochem. 57:67-663 (1988)). Certain plant species accumulate shikimate and shikimate kinase may play a role in regulating flux in the tocopherol pathway.

5-Enolpyruvyl-shikimate-3-phosphate synthase (also known as enolpyruvyl-shikimate-P-synthase, and EPSPS (EC 2.5.1.19)) catalyzes the reversible transfer of the carboxyvinyl moiety of phosphoenolpyruvate to shikimate-3-phosphate, yielding 5-enolpyruvyl-shikimate-3-phosphate. 5-Enolpyruvyl-shikimate-3-phosphate synthase is a target of the broad spectrum, nonselective, postemergence herbicide, glyphosate. Chemical modification studies indicate that lysine, arginine, and histidine residues are essential for activity of the enzyme (Kishore and Shah, Ann. Rev. Biochem. 57:67-663 (1988)). 5-Enolpyruvyl-shikimate-3-phosphate synthase has been isolated and characterized from microbial and plant sources including tomato, petunia, Arabidopsis, and Brassica (Kishore and Shah, Ann. Rev. Biochem, 57:67-663 (1988)).

Chorismate synthase (EC 4.6.1.4) catalyzes the conversion of 5-enolpyruvyl-shikimate-3-phosphate to chorismic acid and introduces a second double bond in an aromatic ring and a trans-1,4-elimination of inorganic phosphorous. Chorismate is the last reported common intermediate in the biosynthesis of aromatic compounds via the shikimate pathway. The enzyme reaction involves no change in the oxidation state of the substrate. Chorismate synthase from various sources requires a reduced flavin cofactor, FMNH2 (reduced flavin mononucleotide) or FADH2 (reduced flavin adenine dinucleotide), for catalytic activity (Bentley, Critical Rev. Biochem. Mol. Biol. 25:307-384 (1990); Kishore and Shah, Ann. Rev. Biochem. 57:67-663 (1988)).

The next reported enzyme in the tocopherol biosynthetic pathway is chorismate mutase (EC 5.4.99.5), which catalyzes the conversion of chorismic acid to prephenic acid. Chorismic acid is a substrate for a number of enzymes involved in the biosynthesis of aromatic compounds. Plant chorismate mutase exists in two isoforms, chorismate mutase-1 and chorismate mutase-2, that differ in feedback regulation by aromatic amino acids (Singh et al., Arch. Biochem. Biophys. 243:374-384 (1985); Goers et al., Planta 162:109-124 (1984)). It has been reported that chloroplastic chorismate mutase-1 may play a role in biosynthesis of aromatic amino acids as this enzyme is activated by tyrosine and phenylalanine. Cytosolic isozyme chorismate mutase-2 is not regulated by aromatic amino acids and may play a role in providing the aromatic nucleus for synthesis of aromatic secondary metabolites including tocopherol (d′Amato et al., Planta, 162:104-108 (1984)).

The metabolic pathways branch after prephenic acid and lead not only to phenylalanine and tyrosine, but also to a number of secondary metabolites. Tyrosine is synthesized from prephenate via either 4-hydroxyphenylpyruvate or arogenate. Both routes have been reported in plants (Bentley, Critical Rev. Biochem. Mol. Biol. 25:307-384 (1990)).

The formation of 4-hydroxyphenylpyruvate from prephenate is catalyzed by prephenate dehydrogenase (EC 1.3.1.12 for NAD specific prephenate dehydrogenase and EC 1.3.1.13 for NADP specific prephenate dehydrogenase). 4-Hydroxyphenylpyruvate associated with tocopherol biosynthesis may also come from tyrosine pool by the action of tyrosine transaminase (EC 2.6.1.5) or L-amino acid oxidase (EC 1.4.3.2). Tyrosine transaminase catalyzes the pyridoxal-phosphate dependent conversion of L-tyrosine to 4-hydroxyphenylpyruvate. This reversible enzyme reaction transfers the amino group of tyrosine to 2-oxoglutarate to form 4-hydroxyphenylpyruvate and glutamate. L-amino acid oxidase (EC 1.4.3.2) catalyzes the conversion of tyrosine to 4-hydroxyphenylpyruvate by acting on the amino group of tyrosine with oxygen acting as an acceptor. L-amino acid oxidase is not specific to tyrosine. In E. coli, aromatic amino acid amino transferase (also referred to as aromatic-amino-acid transaminase (EC 2.6.1.57)) converts 4-hydroxyphenylpyruvate to tyrosine and plays a role in phenylalanine and tyrosine biosynthesis (Oue et al., J. Biochem. (Tokyo) 121:161-171 (1997); Soto-Urzua et al., Can. J. Microbiol. 42:294-298 (1996); Hayashi et al., Biochemistry 32:12229-12239 (1993)).

Aspartic acid amino transferase or transaminase A (EC 2.6.1.1) exhibits a broad substrate specificity and may utilize phenylpyruvate or p-hydroxyphenylpyruvate to form phenylalanine and tyrosine, respectively. Transaminase A has been characterized in Arabidopsis (Wilkie et al., Biochem J. 319:969-976 (1996); Wilkie et al., Plant Mol. Biol. 27:1227-1233 (1995)), rice (Song et al., DNA Res. 3:303-310 (1996)), Panicum miliaceum L (Taniguchi et al., Arch. Biochem. Biophys. 318:295-306 (1995)), Lupinus angustifolius (Winefield et al., Plant Physiol. 104:417-423 (1994)), and soybean (Wadsworth et al., Plant Mol. Biol. 21:993-1009 (1993)).

A precursor molecule, homogentisic acid, is produced in the chloroplast from the shikimate pathway intermediate p-hydroxyphenylpyruvate. p-Hydroxyphenylpyruvate dioxygenase (also known as 4-hydroxyphenylpyruvate dioxygenase (EC 1.13.11.27)) catalyzes the formation of homogentisate from hydroxyphenylpyruvate through an oxidative decarboxylation of the 2-oxoacid side chain accompanied by hydroxylation of the aromatic ring and a 1,2 migration of the carboxymethyl group. Norris et al. reported functional identification of a pdsI gene that encodes p-Hydroxyphenylpyruvate dioxygenase (Norris et al., Plant Cell 7:2139-2149 (1995)). p-Hydroxyphenylpyruvate dioxygenase has been cloned from Arabidopsis and carrot (GenBank accession numbers U89267, AF000228, and U87257; Garcia et al., Biochem. J. 325:761-769 (1997)). Fiedler et al. reported the localization and presence of this enzyme in both isolated spinach chloroplast and the peroxisome (Fiedler et al., Planta, 155:511-515 (1982)). Garcia et al. reported the purification of the cytosolic form of hydroxyphenylpyruvate dioxygenase from cultured carrot protoplast (Garcia et al., Biochem. J. 325:761-769 (1997)). It has been reported that the chloroplastic isoform may be involved in the biosynthesis of prenylquinones, and that the peroxisomal and cytosolic isoform may be involved in the degradation of tyrosine.

The carbon flow to the pool of phytol, i.e., the isoprene-derived side chain of tocopherol, occurs via the mevalonate pathway or non-mevalonate pathway. Geranylgeranyl-pyrophosphate synthase (GGPP synthase (EC 2.5.1.29)) catalyzes the formation of geranylgeranylpyrophosphate by prenyltransferring an isoprene moiety from isopentenylpyrophosphate to farnesylpyrophosphate. A gene encoding geranylgeranyl-pyrophosphate synthase has been isolated from Arabidopsis and Cantharanthus roseus (Zhu et al., Plant Cell Physiol. 38:357-361 (1997), Bantignies et al., Plant Physiol. 110:336-336 (1995)). Geranylgeranylpyrophosphate synthesized by GGPP synthase is used in the carotenoid and tocopherol biosynthesis pathways.

The NADPH-dependent hydrogenation of geranylgeranylpyrophosphate is catalyzed by geranylgeranylpyrophosphate hydrogenase (also called geranylgeranylpyrophosphate reductase) to form phytylpyrophosphate (Soll et al., Plant Physiol. 71:849-854 (1983)). Geranylgeranylpyrophosphate hydrogenase appears to be localized to two sites: the chloroplast envelope and the thylakoids. The chloroplast envelope form is reported to be responsible for the hydrogenation of geranylgeranylpyrophosphate to a phytyl moiety. The thylakoids form is reported to be responsible for the stepwise reduction of chlorophyll esterified with geranylgeraniol to chlorophyll esterified with phytol. The chloroplast envelope form of geranylgeranylpyrophosphate may play a role in tocopherol and phylloquinone synthesis. A chlP gene cloned from Synechocystis has been functionally assigned by complementation in Rhodobactor sphaeroids to catalyze the stepwise hydrogenation of geranylgeraniol to phytol (Addlesse et al., FEBS Lett. 389:126-130 (1996)).

Homogentisate:phytyl transferase (also referred to as phytyl/prenyltransferase) catalyzes the decarboxylation followed by condensation of homogentisic acid with a phytol moiety from phytylpyrophosphate to form 2-methyl-6 phytylbenzoquinol. Prenyltransferase activity has been reported in spinach chloroplasts and such activity is located in chloroplast envelope membranes (Fiedler et al., Planta 155:511-515 (1982)). A reported prenyltransferase gene, termed pdsII, specific to tocopherol biosynthesis has been identified in Arabidopsis (Norris et al., Plant Cell 7:2139-2149 (1995)).

Tocopherol cyclase catalyzes the cyclization of 2,3-dimethyl-6-phytylbenzoquinol to form γ-tocopherol and plays a role in the biosynthesis of enantioselective chromanol substructure of the vitamin E subfamily (Stocker et al., Bioorg. Medic. Chem. 4:1129-1134 (1996)). The preferred substrate specificity of tocopherol cyclase may be either 2,3-dimethyl-6-phytylbenzoquinol or 2-methyl-5-phytylbenzoquinol or both. The substrate, 2-methyl-6 phytylbenzoquinol, is formed by prenyltransferase and requires methylation by an S-adenosylmethionine-dependent methyltransferase before cyclization. Tocopherol cyclase has been purified from green algae chlorella protothecoids, Dunaliella salina and from wheat leafs (U.S. Pat. No. 5,432,069).

Synthesis of γ-tocopherol from 2-methyl-6 phytylbenzoquinol occurs by two pathways with either δ-tocopherol or 2,3 dimethyl-5-phytylbenzoquinol acting as an intermediate. α-Tocopherol is then synthesized from γ-tocopherol in a final methylation step with S-adenosylmethionine. These steps of γ-tocopherol biosynthesis are located in the chloroplast membrane in higher plants. Formation of α-tocopherol from other tocopherols is catalyzed by S-adenosyl methionine (SAM)-dependent γ-tocopherol methyltransferase (EC 2.1.1.95). This enzyme has been partially purified from Capsicum and Euglena gracilis (Shigeoka et al., Biochim. Biophys. Acta 1128:220-226 (1992), d′Harlingue and Camara, J. Biol. Chem. 260:15200-15203 (1985)).

Tocotrienols are similar to tocopherols in molecular structure except that there are three double bonds in the isoprenoid side chain. Although tocotrienols have not been reported in soybean, they are found within in the plant kingdom. The tocotrienol biosynthetic pathway is similar to that of tocopherol up to the formation of homogentisic. It has been reported that homogentisate:phytyl transferase is able to transfer geranylgeranyl-pyrophosphate (“GGPP”) to homogentisic acid. A side chain of GGPP may be desaturated by the addition of phytylpyrophosphate to homogentisate. Stocker et al. report that a reduction of the side chain's double bond occurs at an earlier stage of the biosynthesis. Phytylpyrophosphate or GGPP are condensed with homogentisic acid (“HGA”) to yield different hydroquinone precursors which are cyclized by the same enzyme (Stocker et al., Bioorg. Medicinal Chem. 4:1129-1134 (1996)).

The primary oxidation product of tocopherol is tocopheryl quinone, which can be conjugated to yield glucuronate after prior reduction to the hydroquinone. In animals, glucuronate can be excreted into bile or further catabolized to tocopheronic acid in the kidney and processed for urinary excretion (Traber and Sies, Ann. Rev. Nutr. 16:321-347 (1996)).

In Aspergillus nidulans, the aromatic amino acid catabolic pathway involves formation of homogentisic acid followed by aromatic ring cleavage by an homogentisic acid dioxygenase (EC 1.13.11.5) to yield, after an isomerization step, fumarylacetoacetate (Fernandez-Canon et al., Anal. Biochem. 245:218-22 (1997); Hudecova et al., Int. J. Biochem. Cell Biol. 27:1357-1363 (1995); Fernandez-Canon et al., J. Biol. Chem. 270:21199-21205 (1995)). Fumarylacetoacetate, is then split by fumarylacetoacetate (Fernandez-Canon and Penalva, J. Biol. Chem. 270:21199-21205 (1995)). Homogentisic acid dioxygenase uses a tocopherol biosynthetic metabolite homogentisic acid for hydrolysis.

Tocopherol levels are reported to vary in different plants, tissues, and developmental stages. The production of homogentisic acid by p-hydroxyphenylpyruvate dioxygenase may be a regulatory point for bulk flow through the pathway due to the irreversible nature of the enzyme reaction and due to the fact that homogentisic acid production is the first committed step in tocopherol biosynthesis (Norris et al., Plant Cell 7:2139-2149 (1995)). Another regulatory step in tocopherol biosynthesis may be associated with the availability of phytylpyrophosphate pool. Feeding studies in Safflower callus culture showed 1.8-fold and 18-fold increase in tocopherol synthesis by feeding homogentisate and phytol, respectively (Fury et al., Phytochem. 26:2741-2747 (1987)). In meadow rescue leaf, vitamin E increases in the initial phase of senescence when phytol is cleaved off from the chlorophylls and when a free phytol pool is available (Peskier et al., J. Plant Physiol. 135:428-432 (1989)).

iii. Phytosterol Synthesis Pathway

Phytosterols are a class of natural products that have a tetracyclic ring system. They are synthesized by plants, and algae, via the isoprenoid pathway, which also generates molecules such as carotenoids, gibberellins, terpenes, a phytol side chain of tocopherol, chlorophyll and abscisic acid. Phytosterols can be distinguished from animal sterols (e.g., cholesterol) by the presence of alkyl groups at C-24 in the sterol side chain (Nes and Venkatramesh, Biochemistry and Function of Sterols, ed. Nes and Parish, CRC Press, 111-122 (1997)).

The phytosterol biosynthesis pathway has two distinct components. The early pathway reactions, leading from acetyl-CoA to squalene via mevalonic acid, are common to other isoprenoids. The later pathway reactions, leading from squalene to the major plant sterols such as sitosterol, campesterol and stigmasterol, are committed phytosterol biosynthesis reactions.

These early pathway reactions have been studied in fungi and plants (Lees et al., Biochemistry and Function of Sterols, ed. Nes and Parish, CRC Press, 85-99 (1997); Newman and Chappell, Biochemistry and Function of Sterols, ed. Nes and Parish, CRC Press, 123-134 (1997); Bach et al., Biochemistry and Function of Sterols, ed. Nes and Parish, CRC Press, 135-150 (1997)).

Acetoacetyl CoA thiolase (EC 2.3.1.9) catalyzes the first reported reaction which consists of the formation of acetoacetyl CoA from two molecules of acetyl CoA (Dixon, et al., J. Steroid Biochem. Mol. Biol. 62:165-171 (1997)). This enzyme has been purified from radish. A radish cDNA has been isolated by functional complementation in Saccharomyces cerevisiae (GeneBank Accession # X78116). A radish cDNA has also been screened against a cDNA library of Arabidopsis thaliana (Vollack and Bach, Plant Physiology 111: 1097-1107 (1996)).

HMGCOA synthase (EC 4.1.3.5) catalyzes the production of HMGCoA. This reaction condenses acetyl CoA with acetoacetyl CoA to yield HMGCoA. HMGCoA synthase has been purified from yeast. A plant HMGCoA synthase cDNA has been isolated from Arabidopsis thaliana (Montamat et al., Gene 167:197-201 (1995)).

HMGCoA reductase, also referred to as, 3-hydroxy-3-methyglutaryl-coenzyme A, (EC 1.1.1.34) catalyzes the reductive conversion of HMGCoA to mevalonic acid (MVA). This reaction is reported to play a role in controlling plant isoprenoid biosynthesis (Gray, Adv. Bot. Res. 14:25-91 (1987); Bach et al., Lipids 26:637-648 (1991); Stermer et al., J. Lipid Res. 35:1133-1140 (1994)). Plant HMGCoA reductase genes are often encoded by multigene families. The number of genes comprising each multigene family varies, depending on species, ranging from two in Arabidopsis thaliana to at least seven in potato. Overexpression of plant HMGCoA reductase genes in transgenic tobacco plants has been reported to result in the overproduction of phytosterols (Schaller et al., Plant Physiol. 109:761-770 (1995)).

Mevalonate kinase (EC 2.7.1.36) catalyzes the phosphorylation of mevalonate to produce mevalonate 5-phosphate. It has been reported that mevalonate kinase plays an role in the control of isoprenoid biosynthesis (Lalitha et al., Indian. J. Biochem. Biophys. 23:249-253 (1986)). A mevalonate kinase gene from Arabidopsis thaliana has been cloned (GeneBank accession number X77793; Riou et al., Gene 148:293-297 (1994)).

Phosphomevalonate kinase (EC 2.7.4.2) (MVAP kinase) is an enzyme associated with isoprene and ergosterol biosynthesis that converts mevalonate 5-phosphate to mevalonate 5-pyrophosphate utilizing ATP (Tsay et al., Mol. Cell. Biol. 11:620-631 (1991)).

Mevalonate pyrophosphate decarboxylase (“MVAPP decarboxylase”) (EC 4.1.1.33) catalyses the conversion of mevalonate pyrophosphate to isopentenyl diphosphate (“IPP”). The reaction is reported to be a decarboxylation/dehydration reaction which hydrolyzes ATP and requires Mg²⁺. A cDNA encoding Arabidopsis thaliana MVAPP decarboxylase has been isolated (Toth et al., J. Biol. Chem. 271:7895-7898 (1996)). An isolated Arabidopsis thaliana MVAPP decarboxylase gene was reported to be able to complement the yeast MVAPP decarboxylase.

A second pathway has been reported for the synthesis of IPP from pyruvate without the formation of MVA. This so-called “alternative pathway” or “non-mevalonate pathway” is reported to be the predominant route in the chloroplast for the synthesis of isoprenoids such as terpenes, carotenoids and tocopherol.

Isopentenyl diphosphate isomerase (“IPP:DMAPP”) (EC 5.3.3.2) catalyzes the formation of dimethylallyl pyrophosphate (DMAPP) from isopentenyl pyrophosphate (IPP). Plant IPP:DMAPP isomerase gene sequences have been reported for this enzyme. It has also been reported that IPP:DMAPP isomerase is involved in rubber biosynthesis in a latex extract from Hevea (Tangpakdee et al., Phytochemistry 45:261-267 (1997)).

Farnesyl pyrophosphate synthase (EC 2.5.1.1) is a prenyltransferase which has been reported to play a role in providing polyisoprenoids for sterol biosynthesis as well as a number of other pathways (Li et al., Gene 17:193-196(1996)). Farnesyl pyrophosphate synthase combines DMAPP with IPP to yield geranyl pyrophosphate (“GPP”). The same enzyme condenses GPP with a second molecule of IPP to produce farnesyl pyrophosphate (“FPP”). FPP is a molecule that can proceed down the pathway to sterol synthesis or can be shuttled through other pathways leading to the synthesis of quinones or sesquiterpenes.

Squalene synthase (EC 2.5.1.21) reductively condenses two molecules of FPP in the presence of Mg²⁺ and NADPH to form squalene. The reaction involves a head-to-head condensation and forms a stable intermediate, presqualene diphosphate. The enzyme is subject to sterol demand regulation similar to that of HMGCoA reductase. The activity of squalene synthase has been reported to have a regulatory effect on the incorporation of FPP into sterols and other compounds for which it serves as a precursor (Devarenne et al., Arch. Biochem. Biophys. 349:205-215 (1998)).

Squalene epoxidase (EC 1.14.99.7) (also called squalene monooxygenase) catalyzes the conversion of squalene to squalene epoxide (2,3-oxidosqualene), a precursor to the initial sterol molecule in phytosterol biosynthetic pathway, cycloartenol. This is the first reported step in the pathway where oxygen is required for activity. The formation of squalene epoxide is also the last common reported step in sterol biosynthesis of animals, fungi and plants.

The later pathway of phytosterol biosynthetic steps starts with the cyclization of squalene epoxide and end with the formation of Δ5-24-alkyl sterols in plants.

2,3 oxidosqualene cycloartenol cyclase (EC 5.4.99.8) (also called cycloartenol synthase) is the first step in the sterol pathway that is plant specific. The cyclization of 2,3 oxidosqualene leads to lanosterol in animals and fungi while in plants the product is cycloartenol. Cycloartenol contains a 9,19-cyclopropyl ring. The cyclization is reported to proceed from the epoxy end in a chair-boat-chair-boat sequence that is mediated by a transient C-20 carbocationic intermediate.

S-adenosyl-L-methionine:sterol C-24 methyl transferase (“SMTI”) (EC 2.1.1.41) catalyzes the transfer of a methyl group from a cofactor, S-adenosyl-L-methionine, to the C-24 center of the sterol side chain (Grebenok et al., Plant Mol. Biol. 34:891-896 (1997)). This is the first of two methyl transfer reactions that has been reported to be an obligatory and rate-limiting step of the sterol-producing pathway in plants. The second reaction, a methyl transfer reaction, occurs further down in the pathway after the Δ⁸^{_{—}}⁷ isomerase. Both these methyl transfers are catalyzed by SMTI. An isoform, SMTII, catalyzes the conversion of cycloartenol to a Δ²³⁽²⁴⁾-24-alkyl sterol, cyclosadol.

Sterol C-4 demethylase catalyses the first of several demethylation reactions, which results in the removal of the second methyl groups at C-4. While in animals and fungi the removal of the second C-4 methyl groups occurs consecutively, in plants it has been reported that there are other steps between the first and second C4 demethylations. The C4 demethylation is catalyzed by a complex of microsomal enzymes consisting of a monooxygenase, an NAD⁺-dependent sterol 4-decarboxylase and an NADPH-dependent 3-ketosteroid reductase.

Cycloeucalenol-obtusifoliol isomerase (“COI”) catalyzes the opening of the cyclopropyl ring at C-9. The opening of the cyclopropyl ring at C-9 creates a double bond at C-8.

Sterol C-14 demethylase catalyzes demethylation at C-14 which removes the methyl group at C-14 and creates a double bond at that position. In both fungi and animals this is the first step in the sterol synthesis pathway. Sterol 14-demethylation is mediated by a cytochrome P450 complex.

Sterol C-14 reductase catalyzes a C-14 demethylation that results in the formation of a double bond at C-14 (Ellis et al., Gen. Microbiol. 137:2627-2630 (1991)). This double bond is removed by a Δ¹⁴ reductase. The normal substrate is 4α-methyl-8,14,24 (24¹)-trien-3β-ol. NADPH is the normal reductant.

Sterol C-8 isomerase catalyzes a reaction that involves further modification of the tetracyclic rings or the side chain (Duratti et al., Biochem. Pharmacol. 34:2765-2777 (1985)). Kinetics of the sterolisomerase catalyzed reaction favor a Δ⁸→Δ⁷ isomerase reaction that produces a Δ⁷ group.

Sterol C-5 desaturase catalyzes the insertion of the Δ⁵-double bond that normally occurs at the Δ⁷-sterol level, thereby forming a Δ^{5,7}-sterol (Parks et al., Lipids 30:227-230 (1995)). The reaction has been reported to involve the stereospecific removal of the 5α and 6α hydrogen atoms, biosynthetically derived from the 4 pro-R and 5 pro-S hydrogens of the (+) and (−)R-mevalonic acid, respectively. The reaction is obligatorily aerobic and requires NADPH or NADH. The desaturase has been reported to be a multienzyme complex present in microsomes. It consists of the desaturase itself, cytochrome b₅ and a pyridine nucleotide-dependent flavoprotein. The Δ^{5,7}-desaturase is reported to be a mono-oxygenase that utilizes electrons derived from a reduced pyridine nucleotide via cytochrome b₅.

Sterol C-7 reductase catalyzes the reduction of a r⁷-double bond in r^{5,7}-sterols to generate the corresponding r⁵-sterol. It has been reported that the mechanism involves, like many other sterol enzymes, the formation of a carbocationic intermediate via the electrophilic “attack” by a proton.

Sterol C-24(28) isomerase catalyzes the reduction of a Δ²⁴⁽²⁸⁾→Δ²⁴, a conversion that modifies the side chain. The product is a Δ²⁴⁽²⁵⁾-24-alkyl sterol. Sterol C-24 reductase catalyzes the reduction of the Δ²⁴⁽²⁵⁾ double bond at C-24 which produces sitosterol.

Sterol C-22 desaturase (EC 2.7.3.9) catalyzes the formation of a double bond at C-22 on the side chain. This formation of a double bond at C-22 on the side chain marks the end of the sterol biosynthetic pathway and results in the formation of stigmasterol (Lees et al., Lipids 30:221-226 (1995)). The C-22 desaturase in yeast, which is the reported final step in the biosynthesis of ergosterol in that organism, requires NADPH and molecular oxygen. In addition, the reaction is reported to also involve a cytochrome P450 that is distinct from a cytochrome P450 participating in demethylation reactions.

Phytosterols are biogenetic precursors of brassinosteroids, steroid alkaloids, steroid sapogenins, ecdysteroids and steroid hormones. This precursor role of phytosterols is often described as a “metabolic” function. A common transformation of free sterols in tissues of vascular plants is the conjugation at the 3-hydroxy group of sterols with long-chain fatty acids to form steryl esters, or with a sugar, usually with a single molecule of β-D-glucose, to form steryl glycosides. Some of the steryl glycosides are in addition esterified, at the 6-hydroxy group of the sugar moiety, with long-chain fatty acids to form acylated steryl glycosides.

The presence of several enzymes have been reported that are specifically associated with the synthesis and breakdown of conjugated sterols (Wojciechowski, Physiology and Biochemistry of Sterols, eds. Patterson, Nes, AOCS Press, 361 (1991)). Enzymes involved in this process include: UDPGlc:Sterol glucosyltransferase, phospho(galacto)glyceride steryl glucoside acyltransferase, sterylglycoside and sterylester hydrolases.

UDPGlc:sterol glucosyltransferase (EC 2.4.1.173) catalyzes glucosylation of phytosterols by glucose transfer from UDP-glucose (“UDPGl”). The formation of steryl glycosides can be measured using UDP-[14C]glucose as the substrate. Despite certain differences in their specificity patterns, all reported UDPGlc:sterol glucosyltransferases preferentially glucosylate only sterols or sterol-like molecules that contain a C-3 hydroxy group, a β-configuration and which exhibit a planar ring. It has been reported that UDPGlc:sterol glucosyltransferases are localized in the microsomes.

Phospho(galacto)glyceride steryl glucoside acyltransferase catalyze the formation of acylated steryl glycosides from the substrate steryl glycoside by transfer of acyl groups from some membranous polar acyllipids to steryl glycoside molecules.

Acylglycerol:sterol acyltransferase (EC 2.3.1.26) catalyzes the reaction where certain acylglycerols act as acyl donors in a phytosterol esterification. In plants the activity of acylglycerol:sterol acyltransferase is reported to be associated with membranous fractions. A pronounced specificity for shorter chained unsaturated fatty acids was reported for all acyltransferase preparations studied in plants. For example, acylglycerol:sterol acyltransferases from spinach leaves and mustard roots can esterify a number of phytosterols.

Sterylglycoside and sterylester hydrolases (“SG-hydrolases”) catalyze the enzymatic hydrolysis of sterylglycosides to form free sterols. The SG-hydrolase activity is not found in mature, ungerminated seeds and is reported to emerge only after the third day of germination, and is found mainly in the cotyledons. It has been reported that phospho(galacto)glyceride:SG acyltransferase may catalyze a reversible reaction. Enzymatic hydrolysis of sterylester in germinating seeds of mustard, barley and corn is reported to be low in dormant seeds but increases during the first ten days of germination. This activity is consistent with a decrease in sterylesters and increase in free sterols over the same temporal period.

iv. Brassinosteroids

Brassinosteroids are steroidal compounds with plant growth regulatory properties, including modulation of cell expansion and photomorphogenesis (Artecal, Plant Hormones, Physiology, Biochemistry and Molecular Biology ed. Davies, Kluwer, Academic Publishers, 66 (1995); Yakota, Trends in Plant Science 2:137-143 (1997)). Brassinolide (2α, 3α, 22α, 23α-tetrahydroxy-24-methyl-B-homo-7-oxa-5α-cholestan-6-one) is a biologically active brassinosteroid. More than 40 natural analogs of brassinolide have been reported and these analogues differ primarily in substitutions of the A/B ring system and side chain at position C-17 (Fujioka and Sakurai, Natural Products Report 14:1-10 (1997)).

The pathway leading to brassinolide branches from the synthesis and catabolism of other sterols at campesterol. A synthetic pathway has been reported to campesterol, (24R)-24-methylcholest-4-en-3-one, (24R)-24-5α-methylcholestan-3-one, campestanol, cathasterone, teasterone, 3-dehydroteasterone, typhasterol, castasterone, brassinolide (Fujioka et al., Plant Cell 9:1951-1962 (1997)). An alternative pathway branching from campestanol has also been reported where the 6-oxo group is lacking and not introduced until later in the sequential conversion process. 6-deoxy brassinosteroids have low biological activity and may be catabolic products. However, enzymatic activity converting 6-deoxocastasterone to castasterone has been reported and thus links the alternative pathway to production of bioactive brassinolide.

Two genes encoding BR biosynthetic enzymes have been cloned from Arabidopsis. The earliest acting gene is DET2, which encodes a steroid 5α-reductase with homology to mammalian steroid 5 α-reductases (Li et al., Science 272:398-401 (1996)). The only reductive step in the brassinolide pathway occurs between campesterol and campestanol. A det2 mutation is reported to block the second step in the BR (24R)-24-methylcholest-4-en-3-one to (24R)-24-5-methylcholestan-3-one conversion (Fujioka et al., Plant Cell 9:1951-1962 (1997)).

A second gene, CPD, encodes a cytochrome P450 that has domains homologous to mammalian steroid hydroxylases (Szekeres et al., Cell 85:171-182 (1996)). CPD has been reported to be a teasterone-23-hydroxylase. Mutation of this gene blocks the cathasterone to teasterone conversion. Additional cytochrome P450 enzymes may to participate in brassinolide biosynthesis including the tomato DWARF gene that encodes a P450 cytochrome with 38% identity to CPD (Bishop, Plant Cell 8:959-969 (1996)).

G. Lipid Metabolism

1. β-Oxidation Pathway

The degradation of fatty acids occurs by the β-oxidation pathway. Several inherited human diseases have been reported as genetic deficiencies of β-oxidation enzymes (Roe and Coates, In: Metabolic Basis of Inherited Disease (Scriver et al., eds.) 6^{th} ed. pp. 889-914 (1989)). Fatty acid oxidation is reported in three systems; mitochondrial, peroxisomal and bacterial. Mitochondrial and peroxisomal β-oxidation occurs in animal cells, peroxisomal β-oxidation occurs in plant cells and bacterial β-oxidation is reported to differ from eukaryotic β-oxidation. Peroxisomal β-oxidation is similar to the mitochondrial β-oxidation, except that carnitine has not been reported to be required. In mitochondria, long chain fatty acids are activated by acyl-CoA synthetase on the mitochondrial outer membrane and acyl groups of the CoA esters are transported into the matrix by carnitine acyltransferase. Mitochondrial β-oxidation has been reported as cyclic repetition of four basic reactions catalyzed by a long, medium and short chain acyl-CoA dehydrogenase, an enoyl-CoA hydratase, a 3-hydroxyacyl CoA dehydrogenase and 3-ketoacyl-CoA thiolase. The reported substrates of β-oxidation enzymes are coenzyme A (CoA) derivatives of fatty acid. In peroxisomes, fatty acids have been reported to be activated by acyl-CoA synthetase (Shindo and Hashimoto, J. Biochem. 84:1177-1181 (1978); Krisans et al., J. Biol. Chem. 255:9599-9607 (1980)). Acyl-CoA esters have been reported to be degraded by β-oxidation cycle. β-oxidation has been reported to be catalyzed by acyl-CoA oxidase, enoyl CoA isomerase/enoyl-CoA hydratase/3-hydroxylacyl-CoA dehydrogenase.

Acyl-CoA oxidase (EC 1.3.3.6) is the first reported enzyme of the fatty acid β-oxidation pathways. This enzyme catalyzes the desaturation of acyl-CoAs longer than eight carbons to 2-trans-enoyl-CoAs, by donating electrons directly to molecular oxygen and releasing H₂O₂ (Lazarow et al., 1976). A reported human deficiency of the peroxisomal enzyme results in a lethal disorder called pseudoneonatal adrenoleukodystrophy (Poll—The et al., Am. J. Hum. Genet. 42:422-434 (1988)). Acyl-CoA oxidase isoforms have been reported in human and rat liver (Vanhove et al., J. Biol. Chem. 268:10335-10344 (1993); Schepers et al., J. Biol. Chem. 265:5242-5246 (1990); Osumi et al., J. Biol. Chem. 262:8138-8143 (1987)). Three acyl-CoA oxidases, almitoyl-CoA oxidase, pristanoyl-CoA oxidase and trihydroxycoprostanoyl-CoA oxidase, have been reported to occur within rat liver peroxisomes. Each of the peroxisomal acyl-CoA oxidases is reported to be substrate specific. Acyl-CoA oxidase substrate has been reported as acyl moieties of more than eight carbon atoms (Osumi et al., J. Biochem. 87:1735-1746 (1980)). Clones of rat and human acyl-CoA oxidases have been reported (Osumi et al., J. Biol. Chem. 262:8138-8143 (1987); Reddy et al., Proc. Natl. Acad. Sci. (USA) 84:3214-3218 (1987)). Clones of rat pristanoyl-CoA oxidase and trihydroxycoprostanoyl-CoA oxidase and human branched-chain acyl-CoA oxidase have also been reported (Vanhove et al., J. Biol. Chem. 268:10335-1034 (1993); van Veldhoven et al., Eur. J. Biochem. 222:795-801 (1995)).

Bifunctional protein enoyl-CoA hydratase/3-hydroxyacyl-CoA dehydrogenase is the second reported enzyme of the peroxisomal β-oxidation pathway. Enoyl-CoA hydratase catalyzes hydration of double bond to form 3-L-hydroxyacyl-CoA. 3-hydroxyacyl-CoA dehydrogenase catalyzes NAD⁺ dependent dehydrogenation of β-hydroxy-acyl-CoA resulting in the formation of the corresponding β-ketoacyl-CoA. Originally, bifunctional protein enoyl-CoA hydratase/3-hydroxyacyl-CoA dehydrogenase was reported in rat liver as a monomeric protein with two enzyme activities (Osumi and Hashimoto, Biochem. Biophys. Res. Commun. 89:580-584 (1979)). Enoyl-CoA hydratase/3-hydroxyacyl-CoA dehydrogenase has also been reported as a trifunctional protein with an enoyl-CoA isomerase activity in addition to hydratase and dehydrogenase activity (Palorassi and Hiltunen, J. Biol. Chem. 265:2446-2449 (1990)). Enoyl CoA isomerase/enoyl-CoA hydratase/3-hydroxylacyl-CoA dehydrogenase has also been reported in bovine liver, pig heart and human liver (Fong and Schulz, Methods Enzymol. 71:390-398 (1981); Furuta et al., J. Biochem. 88:1059-1070 (1980); Reddy et al., Proc. Natl. Acad. Sci. (USA) 84:3214-3218 (1987); Osumi and Hashimoto, J. Biol. Chem. 262:8138-8143 (1979)). Rat enoyl-CoA hydratase/3-hydroxyacyl-CoA dehydrogenase/enoyl-CoA isomerase has been reported to contain seven exons. Exons one through five, are reported at the amino terminal to constitute a hydratase domain. 3-hydroxyacyl CoA dehydrogenase activity is reported in exons six and seven. 3-hydroxyacyl CoA dehydrogenase activity has been reported to be present in a 722 amino acid polypeptide (Ishii et al, J. Biol. Chem. 262:8144-8150 (1987); Osumi et al., J. Biol. Chem. 260:8905-8910 (1985)).

3-Ketoacyl-CoA thiolase is reported to catalyze the last step of fatty acid β-oxidation, resulting in Cα-Cβ cleavage yielding acetyl-CoA and new acyl-CoA with two fewer carbons the original one. Two types of mitochondrial thiolases have been reported which differ chain length specificity: 3-ketoacyl CoA thiolase (also known as thiolase I) and acetoacetyl-CoA thiolase (EC 2.3.1.9) (also known as thiolase U). 3-Ketoacyl-CoA-thiolase (EC 2.3.1.16) has reported activity on substrates ranging from acetoacetyl-CoA to long-chain 3-ketoacyl-CoAs at low concentration (Middleton, Methods Enzymol. 35:128-136 (1975; Staack et al, J. Biol. Chem. 253:1827-1931 (1978)). Thiolase has been reported as a tetramer. Rat mitochondrial 3-ketoacyl-CoA thiolase has been reported to have a molecular weight of 41866 Kd (Arakawa et al, EMBO J. 6:1361-1366 (1987)). Peroxisomal 3-ketoacyl-CoA thiolase has been reported in rat liver as a homodimer with a molecular mass of 89 kDa. Mitochondrial 3-ketoacyl-CoA thiolases and mitochondrial and cytosolic acetoacetyl-CoA specific thiolases have been reported as homotetramers, each subunit is about 40 kDa (Miyazawa et al., Eur. J. Biochem. 103:589-596 (1980)). Genes encoding these enzymes have been reported (Hijikata et al., J. Biol. Chem. 262:8151-8158 (1990)). A rat peroxisomal 3-ketoacyl-CoA thiolase and a mitochondrial 3-ketoacyl-CoA thiolase have been reported which contain cysteine residues that are important for substrate binding (Hijikata et al., J. Biol. Chem. 262:8151-8158 (1987); Arakawa et al., EMBO J. 6:1361-1366 (1987)). Thiolases from different species have been reported to have an essential sulfhydryl serving as an acyl acceptor during the thiolytic cleavage (Gilbert et al., J. Biol. Chem. 256:7371-7377 (1981)).

2. Fatty Acid Pathway

In plants, fatty acids are synthesized in the chloroplasts. The pathway is responsible for the formation of fatty acids up to 18 carbons long. The synthesis of fatty acids begins with the reaction between acetyl-CoA and CO₂ to produce malonyl-CoA, which is catalyzed by the enzyme acetyl-CoA carboxylase (ACCase). To form malonyl-Acyl Carrier Proteins (ACP), the transfer of the malonyl moiety from malonyl-CoA is catalyzed by the enzyme malonyl-CoA:ACP transacylase. The first reported elongation step of fatty acid synthesis involves the condensation of a two-carbon unit from malonyl-ACP with acetyl-CoA to form an acetoacetyl-ACP fatty acyl molecule. This reaction is catalyzed by a β-ketoacyl-ACP synthase enzyme which has been designated β-ketoacyl-ACP synthase III (KASIII). Biosynthesis of 16- and 18-carbon fatty acids is followed by the cyclical action of the following sequence of reactions: condensation of acyl-ACP with a two-carbon unit from malonyl-ACP to form elongated β-ketoacyl-ACP (β-ketoacyl-ACP synthase), reduction of the keto-function to an alcohol (β-ketoacyl-ACP reductase), dehydration to form an enoyl-ACP (β-hydroxyacyl-ACP dehydrase), and reduction of an enoyl-ACP to form an elongated saturated acyl-ACP (enoyl-ACP reductase). In plants, this group of dissociated enzymes responsible for the elongation of fatty acids is referred to as fatty acid synthase, or FAS.

Monounsaturated fatty acids are also produced in the plastid, where a double bond can be introduced into the fatty acid molecules by the action of a soluble acyl-ACP desaturase. Acyl-ACPs are substrates for the formation of plastid glycerolipid acids. Alternatively, termination of FAS in the plastids can be catalyzed by acyl-ACP thioesterases (acyl-ACP hydrolases), which hydrolyze acyl-ACPs to form free fatty acids. Free fatty acids may be exported from the chloroplasts to the cytoplasm, where acyl-CoA synthase esterifies the free fatty acids with coenzymeA (CoA) to produce acyl-CoAs. The derived acyl-CoAs are then available as substrates for glycerolipid acid synthesis in the cytoplasm, or in certain plants, for elongation through the action of fatty acid elongase to form longer chain fatty acids.

The FAS pathway in plants has been reviewed (Ohlrogge et al., Fatty Acid Metabolism in Plants, ed. Moore, CRC Press, Boca Raton (1993); Ohlrogge and Browse, Plant Cell 7:957-970 (1995); Harwood, Biochimica et Biophysica Acta 1301:7-56 (1996); Slabas and Fawcett, Plant Molecular Biology 19:169-191 (1992)).

Acetyl-CoA carboxylase, which carries out the first reported committed step in fatty acid synthesis, carboxylates acetyl-CoA to form malonyl-CoA. There are two types of acetyl-CoA carboxylase reported in plants. The first type have been reported to be similar to bacterial enzymes in that it is comprised of four individual and dissociable polypeptides. The four subunits are: biotin carboxylase, biotin carboxyl carrier protein, and two biotin transcarboxylase subunits. The second type of acetyl-CoA carboxylase has been reported to be a large multifunctional protein catalyzing the same three enzyme activities. Dicotyledynous plants and some monocotyledonous plants contain a multisubunit acetyl-CoA carboxylase in their chloroplasts, and a multifunctional acetyl-CoA carboxylase in their cytoplasm. Plants in the graminaceae family have been reported to contain multifunctional acetyl-CoA carboxylase enzymes in both cell compartments.

The first reported partial reaction catalyzed by ACCase is an ATP dependent carboxylation of a biotin prosthetic group of biotin carboxyl carrier protein. Bicarbonate is the primary reported source of carbon and the reaction has been reported to be ATP dependent. The second reported partial reaction is the transfer of the carboxyl group to acetyl-CoA to form malonyl-CoA. A multisubunit enzyme has been partially purified from pea (Alban et al., Biochemistry Journal 300:557-565 (1994)), and certain cDNAs encoding the subunits have been reported from pea and tobacco (Sasaki et al., Journal of Biological Chemistry 268:25118-25123 (1993)). Multifunctional enzymes have been reported from plants and their corresponding genes have been cloned from wheat, maize, Arabidopsis, alfalfa, and rapeseed (Elborough et al., Plant Molecular Biology 24:21-34 (1994); Elborough et al., Biochemistry Journal 301:599-605 (1994); Somers et al., Plant Physiology 101: 1097-1101 (1993); Shorrish et al., Proc. Natl. Acad. Sci. (U.S.A.) 91:4323-4327 (1994)).

Acetyl-CoA:ACP transacylase (“ATA”) catalyzes the transfer of an acetyl moiety from CoA to ACP. This activity has been reported to be a side reaction of KASIII (Tsay et al., Journal of Biological Chemistry 267:6807-6814 (1992)). ATA has been reported to play a minor role in fatty acid synthesis. An ATA enzyme has been partially purified from avocado fruit and pea leaves and the activity has been reported to be separable from KASIII (Harwood, Biochimica et Biophysica Acta 1301:7-56 (1996), Gulliver and Slabas, Plant Molecular Biology 25:179-191 (1994)).

Malonyl CoA:ACP transacylase (MCAT) catalyzes the transfer of a malonyl moiety from CoA to ACP. Malonyl-ACP is the donor of acetate moieties that compose the elongated fatty acyl chains synthesized by FAS. Malonyl-CoA:ACP transacylase has been reported to be present in multiple isoforms and has been partially purified from a number of plant tissues, such as barley (Hoj and Mikkelsen, Carlsberg Research Communication 47:119-141 (1982)), leek (Lessire and Stumpf, Plant Physiology 73:614-618 (1983)), spinach (Stapelton and Jaworski, Biochem. Biophys. Acta 794:240-248 (1984)), and soybean (Guerra and Ohlrogge, Arch. Biochem. Biophys. 246:274-285 (1986)). MCAT has been reported from E. coli (Magnuson et al., FEBS Letters 299:262-266 (1992)).

In plants, FAS elongates fatty acids esterified to ACP. ACP is a small, acidic protein, which has a phosphopantetheine prosthetic group. Genes encoding ACP have been reported in U.S. Pat. Nos. 5,110,728 and 5,315,001. Genes for ACP have also been reported from more than 15 plant species (Ohlrogge et al., Fatty acid Metabolism in Plants, ed. Moore, CRC Press, Boca Raton (1993)). Holo-ACP synthase catalyzes the pantethenylation of apo-ACP using β-alanine as a donor of the panthetheine group. A gene encoding holo-ACP synthase has been reported from E. coli (Lambalot and Walsh, Journal of Biological Chemistry 270:24658-24661 (1995)).

β-Ketoacyl-ACP synthase III, also known as 3-ketoacyl-ACP synthase III and KASIII, catalyzes the first reported condensation reaction of FAS. It condenses acetyl-CoA with malonyl-ACP to form acetoacetyl-ACP. cDNAs encoding KASIII have been reported from spinach (Jaworski et al., Prog. Fatty acid Res. 33:47-54 (1994)), Cuphea (Slabaugh et al., Plant Physiology 108:443-444 (1995)), and Arabidopsis (Tai et al., Plant Physiology 106:801-802 (1994)).

β-Ketoacyl-ACP synthase I and β-ketoacyl-ACP synthase II (also known as 3-ketoacyl-ACP synthase I or KASI and 3-ketoacyl-ACP synthase II or KASII, respectively) catalyze the condensation of the acyl-ACPs with malonyl-ACP to form elongated (by two carbons) β-ketoacyl-ACP. In plants, KASI elongates C4- to C14-ACPs. KASII also elongates the shorter chain substrates. KASII has been reported to exhibit greater activity toward C16-ACP. KASII has been reported to be responsible for the formation of C18 fatty acids. Genes encoding KASI have been reported from barley, castor and rapeseed. Genes encoding a second isoform of the enzyme which has been associated with KASII have been reported from castor and rapeseed (International Patent Application WO 92/03564, U.S. Pat. No. 5,475,099, U.S. Pat. No. 5,510,255).

β-Ketoacyl-ACP reductase, also known as 3-ketoacyl-ACP reductase, reduces the keto group of β-ketoacyl-ACP to form β-hydroxyacyl-ACP. Genes encoding β-ketoacyl-ACP reductase have been reported from Cuphea (Topfer and Martini, Plant Physiology 143:416-425 (1994)), Arabidopsis and rapeseed (Slabas et al., Biochem. Journal 283:321-326 (1992)).

β-Hydroxyacyl-dehydrase (also referred to as β-hydroxyacyl-dehydratase) removes water from β-hydroxyacyl-ACP to form enoyl-ACP. β-Hydroxyacyl-dehydrase has been partially purified from spinach (Shimkata and Stumpf, Arch. Biochem. Biophys. 218:77-91 (1982)) and safflower (Shimkata and Stumpf, Arch. Biochem. Biophys. 217:144-154 (1982)). The gene encoding this enzyme have been reported from E. coli (Cronan et al., Journal of Biological Chemistry 263:4641-4646 (1988); Mohan et al., Journal of Biological Chemistry 269:32896-32903 (1994)).

Enoyl-ACP reductase reduces enoyl-ACP to acyl-ACP. cDNA clones encoding enoyl-ACP reductase have been reported from several plants including rapeseed (Kater et al., Plant Molecular Biology 17:895-909 (1991)) and Cuphea (Walek et al., Biological Chemistry 374:551 (1993)).

Double bonds can be introduced into acyl-ACPs by soluble desaturases. A common chloroplastic desaturase, stearoyl-ACP desaturase, introduces a 9,10 cis double bond into stearoyl-ACP to produce oleoyl-ACP. The reaction has been reported to require oxygen and an electron source. The immediate electron donor has been reported to be ferredoxin. cDNAs encoding stearoyl-ACP desaturase have been reported from a number of plant species including safflower (Thompson et al., Proc. Natl. Acad. Sci. (U.S.A.) 88:2578-2582 (1991); International Patent Application WO 91/13972), castor (Shanklin and Somerville, Proc. Natl. Acad. Sci. (U.S.A.) 88:2510-2514 (1991)), soybean (Chen et al., Plant Physiology 109:1498 (1994)), and spinach (Nishida et al., Plant Molecular Biology 19:711-713 (1992)).

Acyl-ACP thioesterases can terminate FAS. In this reaction thioesterases utilize water to hydrolyze the acyl-ACP thioester linkages to form free fatty acids and ACP. Plants have been reported to harbor two classes of thioesterase (Jones et al., Plant Cell 7:359-371 (1995)). Type A thioesterases (FATA) have been reported to have a substrate preference for oleyl-ACP, and type B thioesterases (FATB) have been reported to have a substrate preference for saturated acyl-ACP. In plant tissues with fatty acid compositions comprising primarily of 16 and 18 carbon fatty acids, FATB enzymes have been reported to have substrate preferences for palmitoyl-ACP (Voelker, Genetic Engineering Vol. 18, ed. Setlow, Plenum Press, New York (1996)).

Free fatty acids released by thioesterases have been reported to be exported from the chloroplast. Acyl-CoA synthetase, an enzyme that has been reported to be associated with the chloroplast envelope, esterifies fatty acids to CoA in an ATP dependent reaction. cDNAs encoding acyl-CoA synthetase have been reported from rapeseed (Fulda et al., Plant Molecular Biology 33:911-922 (1997)).

Fatty acid elongase (FAE (EC 2.3.1.119)) has been reported to be a multisubunit enzyme. Its component enzyme activities have been reported to be similar to FAS. One reported difference between FAE and FAS is that FAE acts on fatty acids esterified to CoA, while FAS acts on fatty acids esterified to ACP. Another reported difference is that FAE enzymes have been reported to be associated with membranes, while FAS enzymes have been reported to be soluble.

β-Ketoacyl-CoA synthase (also known as 3-ketoacyl-CoA synthase and KCS) is a condensing enzyme. KCS catalyzes the elongation step of FAE. KCS uses acyl-CoA and malonyl-CoA as substrates to produce β-ketoacyl-CoA, carbon dioxide, and CoA. cDNAs encoding β-ketoacyl-CoA synthase have been reported from several plant species (Lassner et al., Plant Cell 8:281-292 (1996); U.S. Pat. No. 5,679,881; James et al., Plant Cell 7:309-319 (1995)).

A subsequent reported reaction of FAE is catalyzed by β-ketoacyl-CoA reductase (also known as 3-ketoacyl-CoA reductase). In this NADH or NADPH dependent reaction, β-ketoacyl-CoA is reduced to β-hydroxyacyl CoA. cDNAs encoding β-ketoacyl-CoA reductase have been reported from maize, barley, leek, and Arabidopsis (Xu et al., Plant Physiology 115:501-510 (1997)).

The next reported reaction of FAE is catalyzed by β-hydroxyacyl-CoA dehydrase. This reaction consists of the removal of water from the β-hydroxyacyl-CoA to form enoyl-CoA.

The final reported step of FAE is catalyzed by enoyl-CoA reductase. In this NADH or NADPH dependent reaction, the double bond of enoyl-CoA is reduced to form acyl-CoA.

Glycerolipid acid synthesis has been reviewed (Browse and Somerville, Annual Review of Plant Physiology and Plant Molecular Biology 42:467-506 (1991); Ohlrogge and Browse, Plant Cell 7:957-970 (1995); Harwood, Biochimica et Biophysica Acta 1301:7-56 (1996); Slabas and Fawcett, Plant Molecular Biology 19:169-191 (1992)). Glycerolipid acid synthesis occurs by two reported pathways, a “prokaryotic” pathway and an “eukaryotic” pathway. The term prokaryotic pathway refers to the pathway present in plastids. The term eukaryotic pathway refers to the pathway present in the cytoplasm. The enzymes carrying out the eukaryotic pathway are predominantly associated with microsomal membranes.

The eukaryotic pathway for triglyceride biosynthesis has four reported enzymes: glycerol-3-phosphate O-acyltransferase (EC 2.3.1.15), 1-acyl-glycerol-3 phosphate O-acyltransferase, (EC 2.3.1.51), phosphatidiate phosphatase (EC 3.1.3.4), and diacylglycerol O-acyltransferase (EC 2.3.1.20).

Glycerol-3-phosphate O-acyltransferase (also known as soluble glycerol-3-phosphate acyltransferase (EC 2.3.1.15)) catalyzes the transfer of an acyl group from acyl-CoA to glycerol-3 phosphate to form 1-acyl-glycerol-3 phosphate. The plastid form of this enzyme, which has been reported to be associated with the prokaryotic pathway, utilizes acyl-ACP rather than acyl-CoA as a substrate. A plastid form of this enzyme is soluble and has been reported and cloned from a number of plant species (Murata and Tasaka, Biochimica et Biophysica Acta 1348:10-16 (1997)). Genes encoding membrane forms of the enzyme have been reported from E. coli (Lightner et al., Journal of Biological Chemistry 258:10856-108619 (1983)) and mouse (Shin et al., Journal of Biological Chemistry 266:23834-23839 (1991)).

1-Acyl-glycerol-3 phosphate O-acyltransferase (also known as lysophosphatidic acid acyltransferase (EC 2.3.1.51)) catalyzes the next reported step of triglyceride synthesis. 1-Acyl-glycerol-3 phosphate O-acyltransferase catalyzes the transfer of an acyl group from acyl-CoA to 1-acyl-glycerol-3 phosphate to form phosphatidic acid. A cytoplasmic form of this enzyme has been reported from several species (Lassner et al., Plant Physiology 109:1389-1394 (1995); Knutzon et al., Plant Physiology 109:999-1006 (1995); International Patent Application WO 95/27791). A second reported class of cDNAs that have been reported to encode 1-acyl-glycerol-3 phosphate O-acyltransferase have been reported from maize and rapeseed (Brown et al., Plant Molecular Biology 26:211-223 (1994)).

Phosphatidiate phosphatase (also known as phosphatidic acid phosphatase (EC 3.1.3.4)) hydrolyzes the phosphate group from phosphatidic acid to diacylglycerol. Phosphatidiate phosphatase has been reviewed by Kocsis et al., Lipids 31:785-802 (1996). Phosphatidiate phosphatase has been reported in Saccharomyces cerevisiae and Escherichia coli (Carman et al., Biochim Biophys Acta. 1348:45-55 (1997)).

Diacylglycerol O-acyltransferase (EC 2.3.1.20) catalyzes the final reported step of triglyceride synthesis. Diacylglycerol O-acyltransferase catalyzes the transfer of an acyl group from acyl-CoA to diacylglycerol to form triacylglycerol.

The eukaryotic pathway for phospholipid acid biosynthesis has been reported to have in common the first three enzymes of its biosynthetic pathway with the pathway for triglyceride synthesis: glycerol-3-phosphate O-acyltransferase (EC 2.3.1.15), 1-acyl-glycerol-3 phosphate O-acyltransferase, (EC 2.3.1.51), and phosphatidiate phosphatase (EC 3.1.3.4). There have been multiple reported pathways that attach polar head groups to the sn-3 position of glycerolipid acids to form phospholipid acids. Phosphatidyl choline has been reported to be the most abundant phospholipid acid in plant membranes. Diacylglycerol cholinephosphotransferase (EC 2.7.8.2) catalyzes the transfer of choline from CDP-choline to diacylglycerol to form phosphatidlycholine and CMP. Diacylglycerol cholinephosphotransferase catalyzes the transfer of ethanolamine from CDP-ethanolamine to form phosphatidlyethanolamine (Dewey et al., Plant Cell 6:1495-1507 (1994)). CDP-choline is formed by the enzyme choline-phosphate cytidyltransferase (also known as phosphocholine cytidyltransferase (EC 2.7.7.15)), which utilizes CTP and choline phosphate to form CDP-choline and phosphate. A cDNA encoding choline-phosphate cytidyltransferase has been reported and isolated from rapeseed by complementation of a yeast mutant (Nishida et al., Plant Molecular Biology 31:205-211 (1996)). Choline phosphate is formed from choline and ATP by the enzyme choline kinase (EC 2.7.1.32). A cDNA encoding choline kinase has been reported from soybean (Dewey et al., Plant Physiology 110:1197-1205 (1996)). Phosphatidyl choline can also be formed by the methylation of the ethanolamine head group of phosphatidyl ethanolamine.

Phosphatidyl choline is a substrate for phosphatidyl choline desaturases (EC 1.3.1.35). Phosphatidyl choline desaturases introduce double bonds into oleic acid (omega-6 desaturase) and linoleic acid (omega-3 desaturase). Omega-6 desaturase (also referred to as delta-12 desaturase) incorporates a double bond at the omega-6 position of oleic acid in the sn-2 position of phosphatidyl choline. Omega-3 desaturase (also referred to as delta-6 desaturase) incorporates a double bond at the omega-3 position of linoleic acid in the sn-2 position of phosphatidyl choline. Desaturases have been reported from a number of plant species (Somerville and Browse, Trends in Cell Biology 6:148-153 (1996)). Both desaturases use cytochrome B5 as an electron donor. cDNAs encoding cytochrome B5 have been reported from several plant species including Brassica oleracea (Kearns et al., Plant Physiology 99:1254-1257 (1995)) and tobacco (Smith et al., Plant Molecular Biology 25:527-537 (1994)). Cytochrome B5 reductase has been reported to be required as an electron donor to reduce cytochrome B5 in order for it to donate electrons for the desaturation. A cDNA encoding cytochrome B5 reductase has been reported from human placenta (Yubisui et al., Proc. Natl. Acad. Sci. (U.S.A.) 84:3609-3613 (1987)).

There are several reported mechanisms for exchange of fatty acids between glycerolipid acids. For example, the combined forward and reverse reactions of acyl-CoA:phosphatidyl choline acyltransferase catalyzes an exchange of acyl groups between CoA and phosphatidyl choline (Stymne and Stobart, The Biochemistry of Plants, Vol. 9, ed. Stumpf and Conn, Academic Press, New York (1987)). A second mechanism by which phosphatidyl choline can participate in triacylglycerol synthesis is through the reverse reaction of diacylglycerol cholinephosphotransferase (EC 2.7.8.2).

Phospholipase C (EC 3.1.4.3) can also catalyze the conversion of phosphatidyl choline to diacylglycerol through a hydrolysis reaction. Diacylglycerol kinase synthesizes phosphatidic acid from diacylglycerol in an ATP dependent reaction. A cDNA encoding diacylglycerol kinase has been reported from Arabidopsis (Katagiri et al., Plant Molecular Biology 30:647-653 (1996)). Phospholipase C also hydrolyzes phosphatidyl inositol. A cDNA encoding phospholipase C has been reported from Arabidopsis (Hiayama et al., Proc. Natl. Acad. Sci. (U.S.A.) 92:3903-3907 (1995)).

Phospholipase D hydrolyzes phosphatidyl choline to choline and phosphatidic acid. It has been reported that cDNAs encoding phospholipase D have been reported from castor (Wang et al., Journal of Biological Chemistry 269:20312-20317 (1994)), rice, and corn (Ueki et al., Plant Cell Physiology 36:903-914 (1995)).

Phospholipase A hydrolyzes phosphatidyl choline to lysophosphatidyl choline and free fatty acid. A cDNA encoding phospholipase A2 has been reported (Seilhamer et al., J. Biol. Chem. 264:5335-5338 (1989)). Phospholipase A2 activity has been reported in leaves of Vicia faba (Kim et al., FEBS Lett. 343:213-218 (1994)).

Phospholipase B, also known as lysophospholipase, has been reported to hydrolyze lysophosphatidyl choline to glycerophosphocholine and free fatty acid. Phospholipase B has been reported in leaves of Vicia faba (Kim et al., FEBS Lett. 343:213-218 (1994)).

The products of the prokaryotic pathway for glycerolipid acid synthesis have been reported to be primarily glycolipid acids. Like the eukaryotic pathway, the first three reported enzymes are glycerol-3-phosphate O-acyltransferase, 1-acyl-glycerol-3 phosphate O-acyltransferase, and phosphatidiate phosphatase. Unlike the acyltransferases of the eukaryotic pathway which use acyl-CoAs as substrates, the two plastid acyltransferases use acyl-ACPs as acyl donors. The most abundant chloroplast fatty acid has been reported to be monogalactosyldiacylglycerol (MGDG), which is formed from diacylglycerol by UDP-galactose:diacylglycerol galactosyltransferase. A cDNA encoding this enzyme has been reported from cucumber (Shimojima et al., Proc. Natl. Acad. Sci. (U.S.A.) 94:333-337 (1997)). Digalactosyldiacylglycerol (DGDG), a second major plastid lipid, is synthesized by the dismutation of two molecules of MGDG (Heemskerk et al., Plant Physiology 93:1286-1294 (1990)). Other chloroplast fatty acids include phosphatidyl glycerol (PG) and sulphoquinovosyl diacylglycerol (SL) which are synthesized from phosphatidic acid. Plastids have been reported to have unique omega-3 and omega-6 desaturases. Fatty acid desaturase 4 (FAD4) introduces a trans double bond into the delta-3 position of palmitate esterified to the sn-2 position of PG. FAD5 introduces an omega-9 double bond into palmitate and act specifically on palmitate esterified to the sn-2 position of MGDG. FAD6 desaturates 16:1 and 18:1 esterified to either the sn-1 or sn-2 position of plastid glycerolipid acids and introduces a double bond at the omega-6 position of the fatty acids. FAD7 and FAD8 desaturates 16:2 and 18:3 esterified to either the sn-1 or sn-2 position of plastid glycerolipid acids and introduces a double bond at the omega-3 position of the fatty acids.

Sequence Comparisons

A characteristic feature of a nucleic acid or protein sequence is that it can be compared with other nucleic acid or protein sequences. Sequence comparisons can be undertaken by determining the similarity of the test or query sequence with sequences in publicly available or proprietary databases (“similarity analysis”) or by searching for certain motifs (“intrinsic sequence analysis”)(e.g., cis elements)(Coulson, Trends in Biotechnology 12:76-80 (1994)); Birren et al., Genome Analysis 1: Cold Spring Harbor Laboratory Press, Cold Spring Harbor, N.Y. 543-559 (1997)).

Similarity analysis includes database search and alignment. Examples of public databases include the DNA Database of Japan (DDBJ)(http://www.ddbj.nig.ac.jp/); Genebank (http://www.ncbi.nlm.nih.gov/Web/Search/Index.htlm); and the European Molecular Biology Laboratory Nucleic Acid Sequence Database (EMBL) (http://www.ebi.ac.uk/ebi_docs/embl_db/embl-db.html). Other appropriate databases include dbEST (http://www.ncbi.nlm.nih.gov/dbEST/index.html), SwissProt (http://www.ebi.ac.uk/ebi_docs/swisprot_db/swisshome.html), PIR (http://www-nbrt.georgetown.edu/pir/) and The Institute for Genome Research (http://www.tigr.org/tdb/tdb.html)

A number of different search algorithms have been developed, one example of which are the suite of programs referred to as BLAST programs. There are five implementations of BLAST, three designed for nucleotide sequences queries (BLASTN, BLASTX and TBLASTX) and two designed for protein sequence queries (BLASTP and TBLASTN) (Coulson, Trends in Biotechnology 12:76-80 (1994); Birren et al., Genome Analysis 1, Cold Spring Harbor Laboratory Press, Cold Spring Harbor, N.Y. 543-559 (1997)).

BLASTN takes a nucleotide sequence (the query sequence) and its reverse complement and searches them against a nucleotide sequence database. BLASTN was designed for speed, not maximum sensitivity and may not find distantly related coding sequences. BLASTX takes a nucleotide sequence, translates it in three forward reading frames and three reverse complement reading frames and then compares the six translations against a protein sequence database. BLASTX is useful for sensitive analysis of preliminary (single-pass) sequence data and is tolerant of sequencing errors (Gish and States, Nature Genetics 3:266-272 (1993)). BLASTN and BLASTX may be used in concert for analyzing EST data (Coulson, Trends in Biotechnology 12:76-80 (1994); Birren et al., Genome Analysis 1:543-559 (1997)).

Given a coding nucleotide sequence and the protein it encodes, it is often preferable to use the protein as the query sequence to search a database because of the greatly increased sensitivity to detect more subtle relationships. This is due to the larger alphabet of proteins (20 amino acids) compared with the alphabet of nucleic acid sequences (4 bases), where it is far easier to obtain a match by chance. In addition, with nucleotide alignments, only a match (positive score) or a mismatch (negative score) is obtained, but with proteins, the presence of conservative amino acid substitutions can be taken into account. Here, a mismatch may yield a positive score if the non-identical residue has physical/chemical properties similar to the one it replaced. Various scoring matrices are used to supply the substitution scores of all possible amino acid pairs. A general purpose scoring system is the BLOSUM62 matrix (Henikoff and Henikoff, Proteins 17:49-61 (1993)), which is currently the default choice for BLAST programs. BLOSUM62 is tailored for alignments of moderately diverged sequences and thus may not yield the best results under all conditions. Altschul, J. Mol. Biol. 36:290-300 (1993), describes a combination of three matrices to cover all contingencies. This may improve sensitivity, but at the expense of slower searches. In practice, a single BLOSUM62 matrix is often used but others (PAM40 and PAM250) may be attempted when additional analysis is necessary. Low PAM matrices are directed at detecting very strong but localized sequence similarities, whereas high PAM matrices are directed at detecting long but weak alignments between very distantly related sequences.

Homologues in other organisms are available that can be used for comparative sequence analysis. Multiple alignments are performed to study similarities and differences in a group of related sequences. CLUSTAL W is a multiple sequence alignment package that performs progressive multiple sequence alignments based on the method of Feng and Doolittle, J. Mol. Evol. 25:351-360 (1987). Each pair of sequences is aligned and the distance between each pair is calculated; from this distance matrix, a guide tree is calculated and all of the sequences are progressively aligned based on this tree. A feature of the program is its sensitivity to the effect of gaps on the alignment; gap penalties are varied to encourage the insertion of gaps in probable loop regions instead of in the middle of structured regions. Users can specify gap penalties, choose between a number of scoring matrices, or supply their own scoring matrix for both pairwise alignments and multiple alignments. CLUSTAL W for UNIX and VMS systems is available at: ftp.ebi.ac.uk. Another program is MACAW (Schuler et al., Proteins Struct. Func. Genet. 9:180-190 (1991), for which both Macintosh and Microsoft Windows versions are available. MACAW uses a graphical interface, provides a choice of several alignment algorithms and is available by anonymous ftp at: ncbi.nlm.nih.gov (directory/pub/macaw).

Sequence motifs are derived from multiple alignments and can be used to examine individual sequences or an entire database for subtle patterns. With motifs, it is sometimes possible to detect distant relationships that may not be demonstrable based on comparisons of primary sequences alone. Currently, the largest collection of sequence motifs in the world is PROSITE (Bairoch and Bucher, Nucleic Acid Research 22:3583-3589 (1994)). PROSITE may be accessed via either the ExPASy server on the World Wide Web or anonymous ftp site. Many commercial sequence analysis packages also provide search programs that use PROSITE data.

A resource for searching protein motifs is the BLOCKS E-mail server developed by Henikoff, Trends Biochem Sci. 18:267-268 (1993); Henikoff and Henikoff, Nucleic Acid Research 19:6565-6572 (1991); Henikoff and Henikoff, Proteins 17:49-61 (1993). BLOCKS searches a protein or nucleotide sequence against a database of protein motifs or “blocks.” Blocks are defined as short, ungapped multiple alignments that represent highly conserved protein patterns. The blocks themselves are derived from entries in PROSITE as well as other sources. Either a protein query or a nucleotide query can be submitted to the BLOCKS server; if a nucleotide sequence is submitted, the sequence is translated in all six reading frames and motifs are sought for these conceptual translations. Once the search is completed, the server will return a ranked list of significant matches, along with an alignment of the query sequence to the matched BLOCKS entries.

Conserved protein domains can be represented by two-dimensional matrices, which measure either the frequency or probability of the occurrences of each amino acid residue and deletions or insertions in each position of the domain. This type of model, when used to search against protein databases, is sensitive and usually yields more accurate results than simple motif searches. Two popular implementations of this approach are profile searches such as GCG program ProfileSearch and Hidden Markov Models (HMMs) (Krough et al., J. Mol. Biol. 235:1501-1531, (1994); Eddy, Current Opinion in Structural Biology 6:361-365, (1996)). In both cases, a large number of common protein domains have been converted into profiles, as present in the PROSITE library, or HHM models, as in the Pfam protein domain library (Sonnhammer et al., Proteins 28:405-420 (1997)). Pfam contains more than 500 HMM models for enzymes, transcription factors, signal transduction molecules and structural proteins. Protein databases can be queried with these profiles or HMM models, which will identify proteins containing the domain of interest. For example, HMMSW or HMMFS, two programs in a public domain package called HMMER (Sonnhammer et al., Proteins 28:405-420 (1997)) can be used.

PROSITE and BLOCKS represent collected families of protein motifs. Thus, searching these databases entails submitting a single sequence to determine whether or not that sequence is similar to the members of an established family. Programs working in the opposite direction compare a collection of sequences with individual entries in the protein databases. An example of such a program is the Motif Search Tool, or MoST (Tatusov et al., Proc. Natl. Acad. Sci. (U.S.A.) 91:12091-12095 (1994)). On the basis of an aligned set of input sequences, a weight matrix is calculated by using one of four methods (selected by the user). A weight matrix is simply a representation, position by position of how likely a particular amino acid will appear. The calculated weight matrix is then used to search the databases. To increase sensitivity, newly found sequences are added to the original data set, the weight matrix is recalculated and the search is performed again. This procedure continues until no new sequences are found.

### SUMMARY OF THE INVENTION

The present invention provides a substantially purified nucleic acid molecule where the nucleic acid molecule comprises a nucleic sequence selected from the group consisting of SEQ ID NO: 1 through SEQ ID NO: 294,310 or complements thereof or fragments of either.

The present invention provides a substantially purified first nucleic acid molecule, wherein the first nucleic molecule specifically hybridizes to a second nucleic acid molecule having a nucleic acid sequence selected from the group consisting of SEQ ID NO: 1 through SEQ ID NO: 294,310 or complements thereof.

The present invention provides a marker nucleic acid molecule capable of detecting the level, pattern, occurrence or absence of a biochemical process, wherein the biochemical process is selected from the group consisting of photosynthetic activity, carbohydrate metabolism, amino acid synthesis or degradation, plant hormone or other regulatory molecules, phenolic metabolism, lipid metabolism, biosynthesis of tetrapyrroles, phytochrome metabolism, carbon assimilation, glycolysis metabolism, gluconeogenesis metabolism, sucrose metabolism, starch metabolism, phosphogluconate metabolism, galactomannan metabolism, raffinose metabolism, complex carbohydrate metabolism, phytic acid metabolism, methionine biosynthesis, methionine degradation, lysine metabolism, arginine metabolism, proline metabolism, glutamate/glutamine metabolism, aspartate/asparagine metabolism, cytokinin metabolism, gibberellin metabolism, ethylene metabolism, jasmonic acid metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, fatty acid metabolism, glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

The present invention also provides a substantially purified protein or fragment thereof encoded by a first nucleic acid molecule which specifically hybridizes to a second nucleic acid molecule, the second nucleic acid molecule having a nucleic acid sequence selected from the group consisting of a complement of SEQ ID NO: 1 through SEQ ID NO:294,310.

The present invention also provides a substantially purified protein or fragment thereof encoded by nucleic acid molecule comprising a nucleic acid sequence selected from the group consisting of SEQ ID NO: 1 through SEQ ID NO:294,310.

The present invention also provides a purified antibody or fragment thereof which is capable of specifically binding to a protein or fragment thereof, wherein the protein or fragment thereof is encoded by a nucleic acid molecule comprising a nucleic acid sequence selected from the group consisting of SEQ ID NO: 1 through SEQ ID NO: 294,310.

The present invention also provides a transformed plant having a nucleic acid molecule which comprises: (A) an exogenous promoter region which functions in a plant cell to cause the production of a mRNA molecule; (B) a structural nucleic acid molecule comprising a nucleic acid sequence selected from the group consisting of SEQ ID NO: 1 through SEQ ID NO: 294,310; and (C) a 3′ non-translated sequence that functions in the plant cell to cause termination of transcription and addition of polyadenylated ribonucleotides to a 3′ end of the mRNA molecule.

The present invention also provides a transformed plant having a nucleic acid molecule which comprises: (A) an exogenous promoter region which functions in a plant cell to cause the production of a mRNA molecule; which is linked to (B) a transcribed nucleic acid molecule with a transcribed strand and a non-transcribed strand, wherein the transcribed strand is complementary to a nucleic acid molecule comprising a nucleic acid sequence selected from the group consisting of SEQ ID NO: 1 through SEQ ID NO: 294,310 or fragment thereof; which is linked to (C) a 3′ non-translated sequence that functions in plant cells to cause termination of transcription and addition of polyadenylated ribonucleotides to a 3′ end of the mRNA molecule.

The present invention provides a microarray comprising a collection of nucleic acid molecules wherein the collection of nucleic acid molecules are capable of detecting or predicting a component or attribute of a biochemical process or activity, where the biochemical process or activity are selected from the group consisting of photosynthetic activity, carbohydrate metabolism, amino acid synthesis or degradation, plant hormone or other regulatory molecules, phenolic metabolism, lipid metabolism, biosynthesis of tetrapyrroles, phytochrome metabolism, carbon assimilation, glycolysis metabolism, gluconeogenesis metabolism, sucrose metabolism, starch metabolism, phosphogluconate metabolism, galactomannan metabolism, raffinose metabolism, complex carbohydrate metabolism, phytic acid metabolism, methionine biosynthesis, methionine degradation, lysine metabolism, arginine metabolism, proline metabolism, glutamate/glutamine metabolism, aspartate/asparagine metabolism, cytokinin metabolism, gibberellin metabolism, ethylene metabolism, jasmonic acid metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, fatty acid metabolism, glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

The present invention also provides a method for determining a level or pattern of a plant protein in a plant cell or plant tissue comprising: (A) incubating, under conditions permitting nucleic acid hybridization, a marker nucleic acid molecule having a nucleic acid sequence selected from the group consisting of SEQ ID NO: 1 through SEQ ID NO: 294,310 or complements thereof or fragment of either, with a complementary nucleic acid molecule obtained from the plant cell or plant tissue, wherein nucleic acid hybridization between the marker nucleic acid molecule and the complementary nucleic acid molecule obtained from the plant cell or plant tissue permits the detection of the protein; (B) permitting hybridization between the marker nucleic acid molecule and the complementary nucleic acid molecule obtained from the plant cell or plant tissue; and (C) detecting the level or pattern of the complementary nucleic acid, wherein the detection of the complementary nucleic acid is predictive of the level or pattern of the protein.

The present invention also provides a method for determining a level or pattern of a plant protein in a plant cell or plant tissue comprising: (A) incubating, under conditions permitting nucleic acid hybridization, a marker acid molecule having a nucleic acid sequence selected from the group consisting of SEQ ID NO: 1 through SEQ ID NO: 294,310 or complements thereof or fragment of either, with a complementary nucleic acid molecule obtained from the plant cell or plant tissue, wherein nucleic acid hybridization between the marker nucleic acid molecule and the complementary nucleic acid molecule obtained from the plant cell or plant tissue permits the detection of the protein; (B) permitting hybridization between the marker nucleic acid molecule and the complementary nucleic acid molecule obtained from the plant cell or plant tissue; and (C) detecting the level or pattern of the complementary nucleic acid, wherein the detection of the complementary nucleic acid is predictive of the level or pattern of the protein.

The present invention also provides a method for determining a level or pattern of a protein in a plant cell or plant tissue under evaluation which comprises assaying the concentration of a molecule, whose concentration is dependent upon the expression of a gene, the gene specifically hybridizes to a nucleic acid molecule having a nucleic acid sequence selected from the group consisting of a complement of SEQ ID NO: 1 through SEQ ID NO: 294,310, in comparison to the concentration of that molecule present in a reference plant cell or a reference plant tissue with a known level or pattern of the protein, wherein the assayed concentration of the molecule is compared to the assayed concentration of the molecule in the reference plant cell or reference plant tissue with the known level or pattern of the protein.

The present invention provides a method of determining a mutation in a plant whose presence is predictive of a mutation affecting a level or pattern of a protein comprising the steps: (A) incubating, under conditions permitting nucleic acid hybridization, a marker nucleic acid selected from the group of marker nucleic acid molecules which specifically hybridize to a nucleic acid molecule having a nucleic acid sequence selected from the group of SEQ ID NO: 1 through SEQ ID NO: 294,310 or complements thereof and a complementary nucleic acid molecule obtained from the plant, wherein nucleic acid hybridization between the marker nucleic acid molecule and the complementary nucleic acid molecule obtained from the plant permits the detection of a polymorphism whose presence is predictive of a mutation affecting the level or pattern of the protein in the plant; (3) permitting hybridization between the marker nucleic acid molecule and the complementary nucleic acid molecule obtained from the plant; and (C) detecting the presence of the polymorphism, wherein the detection of the polymorphism is predictive of the mutation.

The present invention also provides a method of producing a plant containing an overexpressed protein comprising: (A) transforming the plant with a functional nucleic acid molecule, wherein the functional nucleic acid molecule comprises a promoter region, wherein the promoter region is linked to a structural region, wherein the structural region has a nucleic acid sequence selected from group consisting of SEQ ID NO: 1 through SEQ ID NO: 294,310; wherein the structural region is linked to a 3′ non-translated sequence that functions in the plant to cause termination of transcription and addition of polyadenylated ribonucleotides to a 3′ end of a mRNA molecule; and wherein the functional nucleic acid molecule results in overexpression of the protein; and (B) growing the transformed plant.

The present invention also provides a method of producing a plant containing reduced levels of a protein comprising: (A) transforming the plant with a functional nucleic acid molecule, wherein the functional nucleic acid molecule comprises a promoter region, wherein the promoter region is linked to a structural region, wherein the structural region comprises a nucleic acid molecule having a nucleic acid sequence selected from the group consisting of nucleic acid sequence selected from the group consisting of a complement of SEQ ID NO: 1 through SEQ ID NO: 294,310 or fragment thereof and the transcribed strand is complementary to an endogenous mRNA molecule; and wherein the transcribed nucleic acid molecule is linked to a 3′ non-translated sequence that functions in the plant cell to cause termination of transcription and addition of polyadenylated ribonucleotides to a 3′ end of a mRNA molecule; and (B) growing the transformed plant.

The present invention also provides a method of determining an association between a polymorphism and a plant trait comprising: (A) hybridizing a nucleic acid molecule specific for the polymorphism to genetic material of a plant, wherein the nucleic acid molecule has a nucleic acid sequence selected from the group consisting of SEQ ID NO: 1 through SEQ ID NO: 294,310 or complements thereof or fragment of either; and (B) calculating the degree of association between the polymorphism and the plant trait.

The present invention also provides a method of isolating a nucleic acid comprising: (A) incubating under conditions permitting nucleic acid hybridization, a first nucleic acid molecule comprising a nucleic acid sequence selected from the group consisting of SEQ ID NO: 1 through SEQ ID NO: 294,310 or complements thereof or fragment of either with a complementary second nucleic acid molecule obtained from a plant cell or plant tissue; (B) permitting hybridization between the first nucleic acid molecule and the second nucleic acid molecule obtained from the plant cell or plant tissue; and (C) isolating the second nucleic acid molecule.

### DETAILED DESCRIPTION OF THE INVENTION

Agents

(a) Nucleic Acid Molecules

Agents of the present invention include plant nucleic acid molecules and more preferably include maize and soybean nucleic acid molecules and more preferably include nucleic acid molecules of the maize genotypes B73 (Illinois Foundation Seeds, Champaign, Ill. U.S.A.), B73×Mo17 (Illinois Foundation Seeds, Champaign, Ill. U.S.A.), DK604 (Dekalb Genetics, Dekalb, Ill. U.S.A.), H99 (USDA Maize Genetic Stock Center, Urbana, Ill. U.S.A.), RX601 (Asgrow Seed Company, Des Moines, Iowa), Mo17 (USDA Maize Genetic Stock Center, Urbana, Ill. U.S.A.), and soybean types Asgrow 3244 (Asgrow Seed Company, Des Moines, Iowa), C1944 (United States Department of Agriculture (USDA) Soybean Germplasm Collection, Urbana, Ill. U.S.A.), Cristalina (USDA Soybean Germplasm Collection, Urbana, Ill. U.S.A.), FT108 (Monsoy, Brazil), Hartwig (USDA Soybean Germplasm Collection, Urbana, Ill. U.S.A.), BW211S Null (Tohoku University, Morioka, Japan), PI507354 (USDA Soybean Germplasm Collection, Urbana, Ill. U.S.A.), Asgrow A4922 (Asgrow Seed Company, Des Moines, Iowa U.S.A.), PI227687 (USDA Soybean Germplasm Collection, Urbana, Ill. U.S.A.), PI229358 (USDA Soybean Germplasm Collection, Urbana, Ill. U.S.A.) and Asgrow A3237 (Asgrow Seed Company, Des Moines, Iowa U.S.A.).

A subset of the nucleic acid molecules of the present invention includes nucleic acid molecules that are marker molecules. Another subset of the nucleic acid molecules of the present invention include nucleic acid molecules that encode a protein or fragment thereof. Another subset of the nucleic acid molecules of the present invention are EST molecules.

Fragment nucleic acid molecules may encode significant portion(s) of, or indeed most of, these nucleic acid molecules. Alternatively, the fragments may comprise smaller oligonucleotides (having from about 15 to about 250 nucleotide residues and more preferably, about 15 to about 30 nucleotide residues, or more preferably about 30 to about 50 nucleotide residues, or again more preferably about 50 to about 100 nucleotide residues).

The term “substantially purified,” as used herein, refers to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic and thus involve the capacity of the agent to mediate a chemical reaction or response.

The agents of the present invention may also be recombinant. As used herein, the term recombinant means any agent (e.g., DNA, peptide, etc.), that is, or results, however indirect, from human manipulation of a nucleic acid molecule.

It is understood that the agents of the present invention may be labeled with reagents that facilitate detection of the agent (e.g., fluorescent labels, Prober et al., Science 238:336-340 (1987); Albarella et al., EP 144914; chemical labels, Sheldon et al., U.S. Pat. No. 4,582,789; Albarella et al., U.S. Pat. No. 4,563,417; modified bases, Miyoshi et al., EP 119448).

It is further understood, that the present invention provides recombinant bacterial, mammalian, microbial, insect, fungal and plant cells and viral constructs comprising the agents of the present invention (See, for example, Exemplary Uses of the Agents of the Invention, Section (a) Plant Constructs and Plant Transformants; Section (b) Fungal Constructs and Fungal Transformants; Section (c) Mammalian Constructs and Transformed Mammalian Cells; Section (d) Insect Constructs and Transformed Insect Cells; Section (e) Bacterial Constructs and Transformed Bacterial Cells; and Section (f) Algal Constructs and Algal Transformants).

Nucleic acid molecules or fragments thereof of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid molecules set forth in SEQ ID NO: 1 through SEQ ID NO: 294,310 or complements thereof under moderately stringent conditions, for example at about 2.0×SSC and about 65° C.

In a particularly preferred embodiment, a nucleic acid of the present invention will include those nucleic acid molecules that specifically hybridize to one or more of the nucleic acid molecules set forth in SEQ ID NO: 1 through SEQ ID NO: 294,310 or complements thereof under high stringency conditions such as 0.2×SSC and about 65° C.

In one aspect of the present invention, the nucleic acid molecules of the present invention comprise one or more of the nucleic acid sequences set forth in SEQ ID NO: 1 through SEQ ID NO: 294,310 or complements thereof or fragments of either. In another aspect of the present invention, one or more of the nucleic acid molecules of the present invention share between 100% and 90% sequence identity with one or more of the nucleic acid sequences set forth in SEQ ID NO: 1 through SEQ ID NO: 294,310 or complements thereof or fragments of either. In a further aspect of the present invention, one or more of the nucleic acid molecules of the present invention share between 100% and 95% sequence identity with one or more of the nucleic acid sequences set forth in SEQ ID NO: 1 through SEQ ID NO: 294,310 or complements thereof or fragments of either. In a more preferred aspect of the present invention, one or more of the nucleic acid molecules of the present invention share between 100% and 98% sequence identity with one or more of the nucleic acid sequences set forth in SEQ ID NO: 1 through SEQ ID NO: 294,310 complements thereof or fragments of either. In an even more preferred aspect of the present invention, one or more of the nucleic acid molecules of the present invention share between 100% and 99% sequence identity with one or more of the sequences set forth in SEQ ID NO: 1 through SEQ ID NO: 294,310 or complements thereof.

In a further more preferred aspect of the present invention, one or more of the nucleic acid molecules of the present invention exhibit 100% sequence identity with a nucleic acid molecule present within MONN01, SATMON001, SATMON003 through SATMON014, SATMON016, SATMON017, SATMON019 through SATMON031, SATMON033, SATMON034, SATMONN01, SATMONN04 through SATMONN06, LIB36, LIB83 through LIB84, CMz029 through CMz031, CMz033 through CMz037, CMz039 through CMz042, CMz044 through CMz045, CMz047 through CMz050, SOYMON001 through SOYMON038, Soy51 through Soy56, Soy58 through Soy62, Soy65 through Soy77, LIB3054, LIB3087, and LIB3094 (Monsanto Company, St. Louis, Mo. U.S.A.).

(i) Nucleic Acid Molecules Encoding Proteins or Fragments Thereof

Nucleic acid molecules of the present invention can comprise sequences that encode a protein or fragment thereof. Such proteins or fragments thereof include homologues of known proteins in other organisms.

In a preferred embodiment of the present invention, a maize or soybean protein or fragment thereof of the present invention is a homologue of another plant protein. In another preferred embodiment of the present invention, a maize or soybean protein or fragment thereof of the present invention is a homologue of a fungal protein. In another preferred embodiment of the present invention, a maize or soybean protein of the present invention is a homologue of mammalian protein. In another preferred embodiment of the present invention, a maize or soybean protein or fragment thereof of the present invention is a homologue of a bacterial protein. In another preferred embodiment of the present invention, a soybean protein or fragment thereof of the present invention is a homologue of a maize protein. In another preferred embodiment of the present invention, a maize protein or fragment thereof of the present invention is a homologue of a soybean protein.

In a preferred embodiment of the present invention, the nucleic molecule of the present invention encodes a protein or fragment thereof where the protein and/or nucleic acid molecule exhibits a BLAST probability score of greater than 1E-12, preferably a BLAST probability score of between about 1E-30 and about 1E-12, even more preferably a BLAST probability score of greater than 1E-30 with its homologue.

In another preferred embodiment of the present invention, the nucleic acid molecule encoding a protein or fragment thereof and/or protein or fragment thereof exhibits a % identity with its homologue of between about 25% and about 40%, more preferably of between about 40 and about 70%, even more preferably of between about 70% and about 90% and even more preferably between about 90% and 99%. In another preferred embodiment of the present invention, the nucleic acid molecule encoding a protein or fragment thereof and/or a protein or fragment thereof exhibits a % identity with its homologue of 100%.

In a preferred embodiment of the present invention, the nucleic molecule of the present invention encodes a protein or fragment thereof where the protein and/or nucleic acid molecule exhibits a BLAST score of greater than 120, preferably a BLAST score of between about 1450 and about 120, even more preferably a BLAST score of greater than 1450 with its homologue.

Nucleic acid molecules of the present invention also include non-maize and non-soybean homologues. Preferred non-maize and non-soybean plant homologues are selected from the group consisting of Arabidopsis, alfalfa, barley, Brassica, broccoli, cabbage, citrus, cotton, garlic, oat, oilseed rape, onion, canola, flax, an ornamental plant, pea, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, oil palm and Phaseolus.

In a preferred embodiment, nucleic acid molecules having SEQ ID NO: 1 through SEQ ID NO: 294,310 or complements and fragments of either can be utilized to obtain such homologues.

The degeneracy of the genetic code, which allows different nucleic acid sequences to code for the same protein or peptide, is known in the literature (U.S. Pat. No. 4,757,006).

In an aspect of the present invention, one or more of the nucleic acid molecules of the present invention differ in nucleic acid sequence from those encoding protein or fragment thereof in SEQ ID NO: 1 through SEQ ID NO: 294,310 due to the degeneracy in the genetic code in that they encode the same protein but differ in nucleic acid sequence.

In another further aspect of the present invention, nucleic acid molecules of the present invention can comprise sequences, which differ from those encoding a protein or fragment thereof in SEQ ID NO: 1 through SEQ ID NO: 294,310 due to fact that the different nucleic acid sequence encodes a protein having one or more conservative amino acid changes. It is understood that codons capable of coding for such conservative amino acid substitutions are known in the art.

It is well known in the art that one or more amino acids in a native sequence can be substituted with another amino acid(s), the charge and polarity of which are similar to that of the native amino acid, i.e., a conservative amino acid substitution, resulting in a silent change. Conserved substitutes for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine.

Conservative amino acid changes within the native polypeptides sequence can be made by substituting one amino acid within one of these groups with another amino acid within the same group. Biologically functional equivalents of the proteins or fragments thereof of the present invention can have 10 or fewer conservative amino acid changes, more preferably seven or fewer conservative amino acid changes, and most preferably five or fewer conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigent-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, obtain a protein with like properties. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157, 105-132 (1982), herein incorporated by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, 1982); these are isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (4.5).

In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference in its entirety, states that the greatest local average hydrophilicity of a protein, as govern by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0±1), glutamate (+3.0±1), serine (+0.3), asparagine (+0.2), glutamine (+0.2), glycine (0), threonine (−0.4), proline (−0.5±1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4). In making such changes, the substitution of amino acids whose hydrophilicity values are within ±2 is preferred, those which are within ±1 are particularly preferred, and those within ±0.5 are even more particularly preferred.

In a further aspect of the present invention, one or more of the nucleic acid molecules of the present invention differ in nucleic acid sequence from those encoding a protein or fragment thereof set forth in SEQ ID NO: 1 through SEQ ID NO: 294,310 or fragment thereof due to the fact that one or more codons encoding an amino acid has been substituted for a codon that encodes a nonessential substitution of the amino acid originally encoded.

A nucleic acid molecule of the present invention can also encode a homologue of a maize or soybean protein. As used herein a homologue protein molecule or fragment thereof is a counterpart protein molecule or fragment thereof in a second species (e.g., maize methionine adenosyltransferase protein is a homologue of Arabidopsis' methionine adenosyltransferase protein).

A homologue can also be generated by molecular evolution or DNA shuffling techniques, so that the molecule retains at least one functional or structure characteristic of the original (see, for example, U.S. Pat. No. 5,811,238).

(ii) Nucleic Acid Molecule Markers and Probes

One aspect of the present invention concerns nucleic acid molecules of the present invention that can act as markers, for example, those nucleic acid molecules SEQ ID NO: 1 through SEQ ID NO: 294,310 or complements thereof or fragments of either that can act as markers or one or more of the marker molecules encoded by other nucleic acid agents of the present invention.

In a preferred embodiment, the level, pattern, occurrence and/or absence of a nucleic acid molecule and/or collection of nucleic acid molecules of the present invention is a marker, for example, for a developmental, commercial or non-commercially valuable trait such as yield or an environmental condition or treatment. It is noted that many agronomic traits can affect yield. These include, without limitation, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits.

As used herein, a “collection of nucleic acid molecules” is a population of nucleic acid molecules where at least two of the nucleic acid molecules differ, at least in part, in their nucleic acid sequence. It is understood, that as used herein, an individual species within a collection of nucleic acid molecules may be physically separate or alternatively not physically separate from one or more other species within the collection of nucleic acid molecules. An example of a situation where individual species may be physically separate but considered a collection of nucleic acid molecules is where more than two species are present on a single support such as a nylon membrane or a glass but occupy a different position on such support. Examples of situations where individual species are physically separate on a support include microarrays.

As used herein, where a collection of nucleic acids is a marker for a particular attribute, the level, pattern, occurrence and/or absence of the nucleic acid molecules associated with the attribute are not required to be the same between species of the collection. For example, the increase in the level of a species when in combination with the decrease in a second species could be diagnostic for a particular attribute.

In an even more preferred embodiment of the present invention, the level, pattern, occurrence and/or absence of a nucleic acid molecule and/or collection of nucleic acid molecules of the present invention is a marker for a biochemical process or activity where the process or activity is preferably selected from photosynthetic activity, carbohydrate metabolism, amino acid synthesis or degradation, plant hormone or other regulatory molecules, phenolic metabolism, and lipid metabolism, and more preferably selected from the group consisting of biosynthesis of tetrapyrroles, phytochrome metabolism, carbon assimilation, glycolysis and gluconeogenesis metabolism, sucrose metabolism, starch metabolism, phosphogluconate metabolism, galactomannan metabolism, raffinose metabolism, complex carbohydrate synthesis/degradation, phytic acid metabolism, methionine biosynthesis, methionine degradation, lysine metabolism, arginine metabolism, proline metabolism, glutamate/glutamine metabolism, aspartate/asparagine metabolism, cytokinin metabolism, gibberellin metabolism, ethylene metabolism, jasmonic acid synthesis metabolism, transcription factors, R-genes, plant proteases, protein kinases, antifungal proteins, nitrogen and sugar transporters, shikimate metabolism, isoflavone metabolism, phenylpropanoid metabolism, isoprenoid metabolism, β-oxidation lipid metabolism, and fatty acid metabolism, and even more preferably selected from the group consisting of: glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

Genetic markers of the present invention include “dominant” or “codominant” markers. “Codominant markers” reveal the presence of two or more alleles (two per diploid individual) at a locus. “Dominant markers” reveal the presence of only a single allele per locus. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominately dimorphic, dominant and codominant markers can be equally valuable. As populations become more heterozygous and multi-allelic, codominant markers often become more informative of the genotype than dominant markers. Marker molecules can be, for example, capable of detecting polymorphisms such as single nucleotide polymorphisms (SNPs).

SNPs are single base changes in genomic DNA sequence. They occur at greater frequency and are spaced with a greater uniformly throughout a genome than other reported forms of polymorphism. The greater frequency and uniformity of SNPs means that there is greater probability that such a polymorphism will be found near or in a genetic locus of interest than would be the case for other polymorphisms. SNPs are located in protein-coding regions and noncoding regions of a genome. Some of these SNPs may result in defective or variant protein expression (e.g., as a results of mutations or defective splicing). Analysis (genotyping) of characterized SNPs can require only a plus/minus assay rather than a lengthy measurement, permitting easier automation.

SNPs can be characterized using any of a variety of methods. Such methods include the direct or indirect sequencing of the site, the use of restriction enzymes (Botstein et al., Am. J. Hum. Genet. 32:314-331 (1980); Konieczny and Ausubel, Plant J. 4:403-410 (1993)), enzymatic and chemical mismatch assays (Myers et al., Nature 313:495-498 (1985)), allele-specific PCR (Newton et al., Nucl. Acids Res. 17:2503-2516 (1989); Wu et al., Proc. Natl. Acad. Sci. (U.S.A.) 86:2757-2760 (1989)), ligase chain reaction (Barany, Proc. Natl. Acad. Sci. (U.S.A.) 88:189-193 (1991)), single-strand conformation polymorphism analysis (Labrune et al., Am. J. Hum. Genet. 48:1115-1120 (1991)), primer-directed nucleotide incorporation assays (Kuppuswami et al., Proc. Natl. Acad. Sci. USA 88:1143-1147 (1991)), dideoxy fingerprinting (Sarkar et al., Genomics 13:441-443 (1992)), solid-phase ELISA-based oligonucleotide ligation assays (Nikiforov et al., Nucl. Acids Res. 22:4167-4175 (1994)), oligonucleotide fluorescence-quenching assays (Livak et al., PCR Methods Appl. 4:357-362 (1995)), 5′-nuclease allele-specific hybridization TaqMan assay (Livak et al., Nature Genet. 9:341-342 (1995)), template-directed dye-terminator incorporation (TDI) assay (Chen and Kwok, Nucl. Acids Res. 25:347-353 (1997)), allele-specific molecular beacon assay (Tyagi et al., Nature Biotech. 16:49-53 (1998)), PinPoint assay (Haff and Smirnov, Genome Res. 7:378-388 (1997)) and dCAPS analysis (Neff et al., Plant J. 14:387-392 (1998)).

Additional markers, such as AFLP markers, RFLP markers and RAPD markers, can be utilized (Walton, Seed World 22-29 (July, 1993); Burow and Blake, Molecular Dissection of Complex Traits, 13-29, Paterson (ed.), CRC Press, New York (1988)). DNA markers can be developed from nucleic acid molecules using restriction endonucleases, the PCR and/or DNA sequence information. RFLP markers result from single base changes or insertions/deletions. These codominant markers are highly abundant in plant genomes, have a medium level of polymorphism and are developed by a combination of restriction endonuclease digestion and Southern blotting hybridization. CAPS are similarly developed from restriction nuclease digestion but only of specific PCR products. These markers are also codominant, have a medium level of polymorphism and are highly abundant in the genome. The CAPS result from single base changes and insertions/deletions.

Another marker type, RAPDs, are developed from DNA amplification with random primers and result from single base changes and insertions/deletions in plant genomes. They are dominant markers with a medium level of polymorphisms and are highly abundant. AFLP markers require using the PCR on a subset of restriction fragments from extended adapter primers. These markers are both dominant and codominant are highly abundant in genomes and exhibit a medium level of polymorphism.

SSRs require DNA sequence information. These codominant markers result from repeat length changes, are highly polymorphic and do not exhibit as high a degree of abundance in the genome as CAPS, AFLPs and RAPDs SNPs also require DNA sequence information. These codominant markers result from single base substitutions. They are highly abundant and exhibit a medium of polymorphism (Rafalski et al., In: Nonmammalian Genomic Analysis, Birren and Lai (ed.), Academic Press, San Diego, Calif., pp. 75-134 (1996)). It is understood that a nucleic acid molecule of the present invention may be used as a marker.

A PCR probe is a nucleic acid molecule capable of initiating a polymerase activity while in a double-stranded structure to with another nucleic acid. Various methods for determining the structure of PCR probes and PCR techniques exist in the art. Computer generated searches using programs such as Primer3 (www-genome.wi.mit.edu/cgi-bin/primer/primer3.cgi), STSPipeline (www-genome.wi.mit.edu/cgi-bin/www-STS Pipeline), or GeneUp (Pesole et al., BioTechniques 25:112-123 (1998)), for example, can be used to identify potential PCR primers.

It is understood that a fragment of one or more of the nucleic acid molecules of the present invention may be a probe and preferably a PCR probe.

(b) Protein and Peptide Molecules

A class of agents comprises one or more of the protein or fragments thereof or peptide molecules encoded by SEQ ID NO: 1 through SEQ ID NO: 294,310 or one or more of the protein or fragment thereof and peptide molecules encoded by other nucleic acid agents of the present invention. As used herein, the term “protein molecule” or “peptide molecule” includes any molecule that comprises five or more amino acids. It is well known in the art that proteins may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, ornithine, homocysteine and homoserine.

Non-limiting examples of the protein or fragment molecules of the present invention are a protein or fragment thereof encoded by: SEQ ID NO: 1 through SEQ ID NO: 294,310 or fragment thereof.

One or more of the protein or fragment of peptide molecules may be produced via chemical synthesis, or more preferably, by expressing in a suitable bacterial or eukaryotic host. Suitable methods for expression are described by Sambrook et al., (In: Molecular Cloning, A Laboratory Manual, 2nd Edition, Cold Spring Harbor Press, Cold Spring Harbor, N.Y. (1989)), or similar texts. For example, the protein may be expressed in, for example, plant, fungal, insect, mammalian and/or bacterial cells (See, for example, Exemplary Uses of the Agents of the Invention, Section (a) Plant Constructs and Plant Transformants; Section (b) Fungal Constructs and Fungal Transformants; Section (c) Mammalian Constructs and Transformed Mammalian Cells; Section (d) Insect Constructs and Transformed Insect Cells; Section (e) Bacterial Constructs and Transformed Bacterial Cells; and Section (f) Algal Constructs and Algal Transformants).

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). Fusion protein or peptide molecules of the present invention are preferably produced via recombinant means.

Another class of agents comprise protein or peptide molecules or fragments or fusions thereof encoded by SEQ ID NO: 1 through SEQ ID NO: 294,310 or fragments thereof in which conservative, non-essential or non-relevant amino acid residues have been added, replaced or deleted. Computerized means for designing modifications in protein structure are known in the art (Dahiyat and Mayo, Science 278:82-87 (1997)).

The protein molecules of the present invention include plant homologue proteins. Plant homologue proteins of the present invention also include non-maize and non-soybean plant homologues. Preferred non-maize, non-soybean, plant homologues are selected from the group consisting of Arabidopsis, alfalfa, barley, Brassica, broccoli, cabbage, citrus, cotton, garlic, oat, oilseed rape, onion, canola, flax, an ornamental plant, pea, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, oil palm and Phaseolus.

Particularly preferred species for use for the isolation of homologues would include, barley, cotton, oat, oilseed rape, rice, canola, ornamentals, sugarcane, sugarbeet, tomato, potato, wheat and turf grasses. Such a homologue can be obtained by any of a variety of methods. Most preferably, as indicated above, one or more of the disclosed sequences (SEQ ID NO: 1 through SEQ ID NO: 294,310 or complements thereof or fragments of either) will be used to define a pair of primers that may be used to isolate the homologue-encoding nucleic acid molecules from any desired species. Such molecules can be expressed to yield homologues by recombinant means.

(c) Antibodies

One aspect of the present invention concerns antibodies, single-chain antigen binding molecules, or other proteins that specifically bind to one or more of the protein or peptide molecules of the present invention and their homologues, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules.

Nucleic acid molecules that encode all or part of the protein of the present invention can be expressed, via recombinant means, to yield protein or peptides that can in turn be used to elicit antibodies that are capable of binding the expressed protein or peptide. Such antibodies may be used in immunoassays for that protein. Such protein-encoding molecules, or their fragments may be a “fusion” molecule (i.e., a part of a larger nucleic acid molecule) such that, upon expression, a fusion protein is produced. It is understood that any of the nucleic acid molecules of the present invention may be expressed, via recombinant means, to yield proteins or peptides encoded by these nucleic acid molecules.

The antibodies that specifically bind proteins and protein fragments of the present invention may be polyclonal or monoclonal and may comprise intact immunoglobulins, or antigen binding portions of immunoglobulins fragments (such as (F(ab′), F(ab′)₂), or single-chain immunoglobulins producible, for example, via recombinant means. It is understood that practitioners are familiar with the standard resource materials which describe specific conditions and procedures for the construction, manipulation and isolation of antibodies (see, for example, Harlow and Lane, In: Antibodies: A Laboratory Manual, Cold Spring Harbor Press, Cold Spring Harbor, N.Y. (1988)).

Murine monoclonal antibodies are particularly preferred. BALB/c mice are preferred for this purpose, however, equivalent strains may also be used. The animals are preferably immunized with approximately 25 μg of purified protein (or fragment thereof) that has been emulsified in a suitable adjuvant (such as TiterMax adjuvant (Vaxcel, Norcross, Ga.)). Immunization is preferably conducted at two intramuscular sites, one intraperitoneal site and one subcutaneous site at the base of the tail. An additional i.v. injection of approximately 25 μg of antigen is preferably given in normal saline three weeks later. After approximately 11 days following the second injection, the mice may be bled and the blood screened for the presence of anti-protein or peptide antibodies. Preferably, a direct binding Enzyme-Linked Immunoassay (ELISA) is employed for this purpose.

More preferably, the mouse having the highest antibody titer is given a third i.v. injection of approximately 25 μg of the same protein or fragment. The splenic leukocytes from this animal may be recovered 3 days later and then permitted to fuse, most preferably, using polyethylene glycol, with cells of a suitable myeloma cell line (such as, for example, the P3X63Ag8.653 myeloma cell line). Hybridoma cells are selected by culturing the cells under “HAT” (hypoxanthine-aminopterin-thymine) selection for about one week. The resulting clones may then be screened for their capacity to produce monoclonal antibodies (“mAbs”), preferably by direct ELISA.

In one embodiment, anti-protein or peptide monoclonal antibodies are isolated using a fusion of a protein or peptide of the present invention, or conjugate of a protein or peptide of the present invention, as immunogens. Thus, for example, a group of mice can be immunized using a fusion protein emulsified in Freund's complete adjuvant (e.g. approximately 50 μg of antigen per immunization). At three week intervals, an identical amount of antigen is emulsified in Freund's incomplete adjuvant and used to immunize the animals. Ten days following the third immunization, serum samples are taken and evaluated for the presence of antibody. If antibody titers are too low, a fourth booster can be employed. Polysera capable of binding the protein or peptide can also be obtained using this method.

In a preferred procedure for obtaining monoclonal antibodies, the spleens of the above-described immunized mice are removed, disrupted and immune splenocytes are isolated over a ficoll gradient. The isolated splenocytes are fused, using polyethylene glycol with BALB/c-derived HGPRT (hypoxanthine guanine phosphoribosyl transferase) deficient P3x63xAg8.653 plasmacytoma cells. The fused cells are plated into 96 well microtiter plates and screened for hybridoma fusion cells by their capacity to grow in culture medium supplemented with hypothanthine, aminopterin and thymidine for approximately 2-3 weeks.

Hybridoma cells that arise from such incubation are preferably screened for their capacity to produce an immunoglobulin that binds to a protein of interest. An indirect ELISA may be used for this purpose. In brief, the supernatants of hybridomas are incubated in microtiter wells that contain immobilized protein. After washing, the titer of bound immunoglobulin can be determined using, for example, a goat anti-mouse antibody conjugated to horseradish peroxidase. After additional washing, the amount of immobilized enzyme is determined (for example through the use of a chromogenic substrate). Such screening is performed as quickly as possible after the identification of the hybridoma in order to ensure that a desired clone is not overgrown by non-secreting neighbor cells. Desirably, the fusion plates are screened several times since the rates of hybridoma growth vary. In a preferred sub-embodiment, a different antigenic form may be used to screen the hybridoma. Thus, for example, the splenocytes may be immunized with one immunogen, but the resulting hybridomas can be screened using a different immunogen. It is understood that any of the protein or peptide molecules of the present invention may be used to raise antibodies.

As discussed below, such antibody molecules or their fragments may be used for diagnostic purposes. Where the antibodies are intended for diagnostic purposes, it may be desirable to derivatize them, for example with a ligand group (such as biotin) or a detectable marker group (such as a fluorescent group, a radioisotope or an enzyme).

The ability to produce antibodies that bind the protein or peptide molecules of the present invention permits the identification of mimetic compounds of those molecules. A “mimetic compound” is a compound that is not that compound, or a fragment of that compound, but which nonetheless exhibits an ability to specifically bind to antibodies directed against that compound.

It is understood that any of the agents of the present invention can be substantially purified and/or be biologically active and/or recombinant.

Exemplary Uses of the Agents of the Invention

Nucleic acid molecules and fragments thereof of the present invention may be employed to obtain other nucleic acid molecules from the same species (e.g., ESTs or fragments thereof from maize may be utilized to obtain other nucleic acid molecules from maize). Such nucleic acid molecules include the nucleic acid molecules that encode the complete coding sequence of a protein and promoters and flanking sequences of such molecules. In addition, such nucleic acid molecules include nucleic acid molecules that encode for other isozymes or gene family members. Such molecules can be readily obtained by using the above-described nucleic acid molecules or fragments thereof to screen cDNA or genomic libraries obtained from maize or soybean. Methods for forming such libraries are well known in the art.

Nucleic acid molecules and fragments thereof of the present invention may also be employed to obtain nucleic acid homologues. Such homologues include the nucleic acid molecule of other plants or other organisms (e.g., Arabidopsis, alfalfa, barley, broccoli, cabbage, citrus, cotton, garlic, oat, oilseed rape, onion, canola, flax, an ornamental plant, pea, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, oil palm, Phaseolus, etc.) including the nucleic acid molecules that encode, in whole or in part, protein homologues of other plant species or other organisms, sequences of genetic elements such as promoters and transcriptional regulatory elements.

Such molecules can be readily obtained by using the above-described nucleic acid molecules or fragments thereof to screen cDNA or genomic libraries obtained from such plant species. Methods for forming such libraries are well known in the art. Such homologue molecules may differ in their nucleotide sequences from those found in one or more of SEQ ID NO: 1 through SEQ ID NO: 294,310 or complements thereof or fragments of either because complete complementarity is not needed for stable hybridization. The nucleic acid molecules of the present invention therefore also include molecules that, although capable of specifically hybridizing with the nucleic acid molecules may lack “complete complementarity.”

Any of a variety of methods may be used to obtain one or more of the above-described nucleic acid molecules (Zamechik et al., Proc. Natl. Acad. Sci. (U.S.A.) 83:4143-4146 (1986); Goodchild et al., Proc. Natl. Acad. Sci. (U.S.A.) 85:5507-5511 (1988); Wickstrom et al., Proc. Natl. Acad. Sci. (U.S.A.) 85:1028-1032 (1988); Holt et al., Molec. Cell. Biol. 8:963-973 (1988); Gerwirtz et al., Science 242:1303-1306 (1988); Anfossi et al., Proc. Natl. Acad. Sci. (U.S.A.) 86:3379-3383 (1989); Becker et al., EMBO J. 8:3685-3691 (1989)). Automated nucleic acid synthesizers may be employed for this purpose. In lieu of such synthesis, the disclosed nucleic acid molecules may be used to define a pair of primers that can be used with the polymerase chain reaction (Mullis et al., Cold Spring Harbor Symp. Quant. Biol. 51:263-273 (1986); Erlich et al., European Patent 50,424; European Patent 84,796; European Patent 258,017; European Patent 237,362; Mullis, European Patent 201,184; Mullis et al., U.S. Pat. No. 4,683,202; Erlich, U.S. Pat. No. 4,582,788; and Saiki et al., U.S. Pat. No. 4,683,194) to amplify and obtain any desired nucleic acid molecule or fragment.

Promoter sequence(s) and other genetic elements, including but not limited to transcriptional regulatory flanking sequences, associated with one or more of the disclosed nucleic acid sequences can also be obtained using the disclosed nucleic acid sequence provided herein. In one embodiment, such sequences are obtained by incubating EST nucleic acid molecules or preferably fragments thereof with members of genomic libraries (e.g. maize and soybean) and recovering clones that hybridize to the EST nucleic acid molecule or fragment thereof. In a second embodiment, methods of “chromosome walking,” or inverse PCR may be used to obtain such sequences (Frohman et al., Proc. Natl. Acad. Sci. (U.S.A.) 85:8998-9002 (1988); Ohara et al., Proc. Natl. Acad. Sci. (U.S.A.) 86:5673-5677 (1989); Pang et al., Biotechniques 22:1046-1048 (1997); Huang et al., Methods Mol. Biol. 69:89-96 (1997); Huang et al., Method Mol. Biol. 67:287-294 (1997); Benkel et al., Genet. Anal. 13:123-127 (1996); Hartl et al., Methods Mol. Biol. 58:293-301 (1996)).

The nucleic acid molecules of the present invention may be used to isolate promoters of cell enhanced, cell specific, tissue enhanced, tissue specific, developmentally or environmentally regulated expression profiles. Isolation and functional analysis of the 5′ flanking promoter sequences of these genes from genomic libraries, for example, using genomic screening methods and PCR techniques would result in the isolation of useful promoters and transcriptional regulatory elements. These methods are known to those of skill in the art and have been described (See, for example, Birren et al., Genome Analysis: Analyzing DNA, 1, Cold Spring Harbor Laboratory Press, Cold Spring Harbor, N.Y., (1997); Birren et al., Genome Analysis: Detecting Genes, 2, (1998), Cold Spring Harbor Laboratory Press, Cold Spring Harbor, N.Y., (1998); Birren et al., Genome Analysis: Cloning Systems, 3, Cold Spring Harbor Laboratory Press, Cold Spring Harbor, N.Y., (1999); Birren et al., Genome Analysis: Mapping Genomes, 4, Cold Spring Harbor Laboratory Press, Cold Spring Harbor, N.Y., (1999). Promoters obtained utilizing the nucleic acid molecules of the present invention could also be modified to affect their control characteristics. Examples of such modifications would include but are not limited to enhanced sequences as reported in Exemplary Uses of the Agents of the Invention, Section (a) Plant Constructs and Plant Transformants. Such genetic elements could be used to enhance gene expression of new and existing traits for crop improvements.

In one sub-aspect, such an analysis is conducted by determining the presence and/or identity of polymorphism(s) by one or more of the nucleic acid molecules of the present invention and more preferably one or more of the EST nucleic acid molecule or complement thereof or fragment of either which are associated with a phenotype, or a predisposition to that phenotype.

Any of a variety of molecules can be used to identify such polymorphism(s). In one embodiment, one or more of the EST nucleic acid molecules (or completement thereof or a sub-fragment of either) may be employed as a marker nucleic acid molecule to identify such polymorphism(s). Alternatively, such polymorphisms can be detected through the use of a marker nucleic acid molecule or a marker protein that is genetically linked to (i.e., a polynucleotide that co-segregates with) such polymorphism(s).

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s) and more preferably within 100 kb of the polymorphism(s) and most preferably within 10 kb of the polymorphism(s) can be employed.

The genomes of animals and plants naturally undergo spontaneous mutation in the course of their continuing evolution (Gusella, Ann. Rev. Biochem. 55:831-854 (1986)). A “polymorphism” is a variation or difference in the sequence of the gene or its flanking regions that arises in some of the members of a species. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist and the polymorphism is thus said to be di-allelic. In other cases, the species' population may contain multiple alleles and the polymorphism is termed tri-allelic, etc. A single gene may have multiple different unrelated polymorphisms. For example, it may have a di-allelic polymorphism at one site and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRs) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity analysis (Weber, U.S. Pat. No. 5,075,217; Armour et al., FEBS Lett. 307:113-115 (1992); Jones et al., Eur. J. Haematol. 39:144-147 (1987); Horn et al., PCT Patent Application WO91/14003; Jeffreys, European Patent Application 370,719; Jeffreys, U.S. Pat. No. 5,175,082; Jeffreys et al., Amer. J. Hum. Genet. 39:11-24 (1986); Jeffreys et al., Nature 316:76-79 (1985); Gray et al., Proc. R. Acad. Soc. Lond. 243:241-253 (1991); Moore et al., Genomics 10:654-660 (1991); Jeffreys et al., Anim. Genet. 18:1-15 (1987); Hillel et al., Anim. Genet. 20:145-155 (1989); Hillel et al., Genet. 124:783-789 (1990)).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

The most preferred method of achieving such amplification employs the polymerase chain reaction (“PCR”) (Mullis et al., Cold Spring Harbor Symp. Quant. Biol. 51:263-273 (1986); Erlich et al., European Patent Appln. 50,424; European Patent Appln. 84,796; European Patent Application 258,017; European Patent Appln. 237,362; Mullis, European Patent Appln. 201,184; Mullis et al., U.S. Pat. No. 4,683,202; Erlich, U.S. Pat. No. 4,582,788; and Saiki et al., U.S. Pat. No. 4,683,194), using primer pairs that are capable of hybridizing to the proximal sequences that define a polymorphism in its double-stranded form.

In lieu of PCR, alternative methods, such as the “Ligase Chain Reaction” (“LCR”) may be used (Barany, Proc. Natl. Acad. Sci. (U.S.A.) 88:189-193 (1991)). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide. Alternatively, the oligonucleotides may be selected such that they do not include the polymorphic site (see, Segev, PCT Application WO 90/01069).

The “Oligonucleotide Ligation Assay” (“OLA”) may alternatively be employed (Landegren et al., Science 241:1077-1080 (1988)). The OLA protocol uses two oligonucleotides which are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson et al., have described a nucleic acid detection assay that combines attributes of PCR and OLA (Nickerson et al., Proc. Natl. Acad. Sci. (U.S.A.) 87:8923-8927 (1990)). In this method, PCR is used to achieve the exponential amplification of target DNA, which is then detected using OLA. In addition to requiring multiple and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide”, thereby amplifying the di-oligonucleotide, are also known (Wu et al., Genomics 4:560-569 (1989)) and may be readily adapted to the purposes of the present invention.

Other known nucleic acid amplification procedures, such as allele-specific oligomers, branched DNA technology, transcription-based amplification systems, or isothermal amplification methods may also be used to amplify and analyze such polymorphisms (Malek et al., U.S. Pat. No. 5,130,238; Davey et al., European Patent Application 329,822; Schuster et al., U.S. Pat. No. 5,169,766; Miller et al., PCT Patent Application WO 89/06700; Kwoh et al., Proc. Natl. Acad. Sci. (U.S.A.) 86:1173-1177 (1989); Gingeras et al., PCT Patent Application WO 88/10315; Walker et al., Proc. Natl. Acad. Sci. (U.S.A.) 89:392-396 (1992)).

The identification of a polymorphism can be determined in a variety of ways. By correlating the presence or absence of it in a plant with the presence or absence of a phenotype, it is possible to predict the phenotype of that plant. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and plant genetic analyses (Glassberg, UK Patent Application 2135774; Skolnick et al., Cytogen. Cell Genet. 32:58-67 (1982); Botstein et al., Ann. J. Hum. Genet. 32:314-331 (1980); Fischer et al., PCT Application WO90/13668; Uhlen, PCT Application WO90/11369).

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine Molecular Diagnosis of Genetic Diseases, Humana Press (1996); Orita et al., Genomics 5:874-879 (1989)). Under denaturing conditions a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis. A number of protocols have been described for SSCP including, but not limited to, Lee et al., Anal. Biochem. 205:289-293 (1992); Suzuki et al., Anal. Biochem. 192:82-84 (1991); Lo et al., Nucleic Acids Research 20:1005-1009 (1992); Sarkar et al., Genomics 13:441-443 (1992). It is understood that one or more of the nucleic acids of the present invention, may be utilized as markers or probes to detect polymorphisms by SSCP analysis.

Polymorphisms may also be found using a DNA fingerprinting technique called amplified fragment length polymorphism (AFLP), which is based on the selective PCR amplification of restriction fragments from a total digest of genomic DNA to profile that DNA (Vos et al., Nucleic Acids Res. 23:4407-4414 (1995)). This method allows for the specific co-amplification of high numbers of restriction fragments, which can be visualized by PCR without knowledge of the nucleic acid sequence.

AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel.

AFLP analysis has been performed on Salix (Beismann et al., Mol. Ecol. 6:989-993 (1997)), Acinetobacter (Janssen et al., Int. J. Syst. Bacteriol. 47:1179-1187 (1997)), Aeromonas popoffi (Huys et al., Int. J. Syst. Bacteriol. 47:1165-1171 (1997)), rice (McCouch et al., Plant Mol. Biol. 35:89-99 (1997); Nandi et al., Mol. Gen. Genet. 255:1-8 (1997); Cho et al., Genome 39:373-378 (1996)), barley (Hordeum vulgare) (Simons et al., Genomics 44:61-70 (1997); Waugh et al., Mol. Gen. Genet. 255:311-321 (1997); Qi et al., Mol. Gen. Genet. 254:330-336 (1997); Becker et al., Mol. Gen. Genet. 249:65-73 (1995)), potato (Van der Voort et al., Mol. Gen. Genet. 255:438-447 (1997); Meksem et al., Mol. Gen. Genet. 249:74-81 (1995)), Phytophthora infestans (Van der Lee et al., Fungal Genet. Biol. 21:278-291 (1997)), Bacillus anthracis (Keim et al., J. Bacteriol. 179:818-824 (1997)), Astragalus cremnophylax (Travis et al., Mol. Ecol. 5:735-745 (1996)), Arabidopsis (Cnops et al., Mol. Gen. Genet. 253:32-41 (1996)), Escherichia coli (Lin et al., Nucleic Acids Res. 24:3649-3650 (1996)), Aeromonas (Huys et al., Int. J. Syst. Bacteriol. 46:572-580 (1996)), nematode (Folkertsma et al., Mol. Plant. Microbe Interact. 9:47-54 (1996)), tomato (Thomas et al., Plant J. 8:785-794 (1995)) and human (Latorra et al., PCR Methods Appl. 3:351-358 (1994)). AFLP analysis has also been used for fingerprinting mRNA (Money et al., Nucleic Acids Res. 24:2616-2617 (1996); Bachem et al., Plant J. 9:745-753 (1996)). It is understood that one or more of the nucleic acids of the present invention, may be utilized as markers or probes to detect polymorphisms by AFLP analysis or for fingerprinting RNA.

Polymorphisms may also be found using random amplified polymorphic DNA (RAPD) (Williams et al., Nucl. Acids Res. 18:6531-6535 (1990)) and cleaveable amplified polymorphic sequences (CAPS) (Lyamichev et al., Science 260:778-783 (1993)). It is understood that one or more of the nucleic acid molecules of the present invention, may be utilized as markers or probes to detect polymorphisms by RAPD or CAPS analysis.

Through genetic mapping, a fine scale linkage map can be developed using DNA markers and, then, a genomic DNA library of large-sized fragments can be screened with molecular markers linked to the desired trait. Molecular markers are advantageous for agronomic traits that are otherwise difficult to tag, such as resistance to pathogens, insects and nematodes, tolerance to abiotic stress, quality parameters and quantitative traits such as high yield potential.

The essential requirements for marker-assisted selection in a plant breeding program are: (1) the marker(s) should co-segregate or be closely linked with the desired trait; (2) an efficient means of screening large populations for the molecular marker(s) should be available; and (3) the screening technique should have high reproducibility across laboratories and preferably be economical to use and be user-friendly.

The genetic linkage of marker molecules can be established by a gene mapping model such as, without limitation, the flanking marker model reported by Lander and Botstein, Genetics 121:185-199 (1989) and the interval mapping, based on maximum likelihood methods described by Lander and Botstein, Genetics 121:185-199 (1989) and implemented in the software package MAPMAKER/QTL (Lincoln and Lander, Mapping Genes Controlling Quantitative Traits Using MAPMAKER/QTL, Whitehead Institute for Biomedical Research, Massachusetts (1990). Additional software includes Qgene, Version 2.23, Department of Plant Breeding and Biometry, 266 Emerson Hall, Cornell University, Ithaca, N.Y. (1996). Use of Qgene software is a particularly preferred approach.

A maximum likelihood estimate (MLE) for the presence of a marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL).

The LOD score essentially indicates how much more likely the data are to have arisen assuming the presence of a QTL than in its absence. The LOD threshold value for avoiding a false positive with a given confidence, say 95%, depends on the number of markers and the length of the genome. Graphs indicating LOD thresholds are set forth in Lander and Botstein, Genetics 121:185-199 (1989), and further described by Arús and Moreno-González, Plant Breeding, Hayward et al., (eds.) Chapman & Hall, London, pp. 314-331 (1993).

Additional models can be used. Many modifications and alternative approaches to interval mapping have been reported, including the use non-parametric methods (Kruglyak and Lander, Genetics 139:1421-1428 (1995)). Multiple regression methods or models can be also be used, in which the trait is regressed on a large number of markers (Jansen, Biometrics in Plant Breeding, van Oijen and Jansen (eds.), Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval and at the same time onto a number of markers that serve as ‘cofactors,’ have been reported by Jansen and Stam, Genetics 136:1447-1455 (1994), and Zeng, Genetics 136:1457-1468 (1994). Generally, the use of cofactors reduces the bias and sampling error of the estimated QTL positions (Utz and Melchinger, Biometrics in Plant Breeding, van Oijen and Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng, Genetics 136:1457-1468 (1994)). These models can be extended to multi-environment experiments to analyze genotype-environment interactions (Jansen et al., Theo. Appl. Genet. 91:33-37 (1995)).

Selection of an appropriate mapping populations is important to map construction. The choice of appropriate mapping population depends on the type of marker systems employed (Tanksley et al., Molecular mapping plant chromosomes. Chromosome structure and function: Impact of new concepts, Gustafson and Appels (eds.), Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

An F₂ population is the first generation of selfing after the hybrid seed is produced. Usually a single F₁ plant is selfed to generate a population segregating for all the genes in Mendelian (1:2:1) fashion. Maximum genetic information is obtained from a completely classified F₂ population using a codominant marker system (Mather, Measurement of Linkage in Heredity, Methuen and Co., (1938)). In the case of dominant markers, progeny tests (e.g., F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g., disease resistance) or where trait expression is controlled by a QTL. Segregation data from progeny test populations (e.g., F₃ or BCF₂) can be used in map construction. Marker-assisted selection can then be applied to cross progeny based on marker-trait map associations (F₂, F₃), where linkage groups have not been completely disassociated by recombination events (i.e., maximum disequillibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant markers evaluated in RIL populations provide more information per individual than either marker type in backcross populations (Reiter et al., Proc. Natl. Acad. Sci. (U.S.A.) 89:1477-1481 (1992)). However, as the distance between markers becomes larger (i.e., loci become more independent), the information in RIL populations decreases dramatically when compared to codominant markers.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts or mosaic of genomic regions from the donor parent. Backcross populations can be useful for mapping dominant markers if all loci in the recurrent parent are homozygous and the donor and recurrent parent have contrasting polymorphic marker alleles (Reiter et al., Proc. Natl. Acad. Sci. (U.S.A.) 89:1477-1481 (1992)). Information obtained from backcross populations using either codominant or dominant markers is less than that obtained from F₂ populations because one, rather than two, recombinant gametes are sampled per plant. Backcross populations, however, are more informative (at low marker saturation) when compared to RILs as the distance between linked loci increases in RIL populations (i.e., about 15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under interrogation can be used as a mapping population. In mapping with NILs, only a portion of the polymorphic loci are expected to map to a selected region.

Bulk segregant analysis (BSA) is a method developed for the rapid identification of linkage between markers and traits of interest (Michelmore et al., Proc. Natl. Acad. Sci. (U.S.A.) 88:9828-9832 (1991)). In BSA, two bulked DNA samples are drawn from a segregating population originating from a single cross. These bulks contain individuals that are identical for a particular trait (resistant or susceptible to particular disease) or genomic region but arbitrary at unlinked regions (i.e., heterozygous). Regions unlinked to the target region will not differ between the bulked samples of many individuals in BSA.

It is understood that one or more of the nucleic acid molecules of the present invention may be used as molecular markers. It is also understood that one or more of the protein molecules of the present invention may be used as molecular markers.

In accordance with this aspect of the present invention, a sample nucleic acid is obtained from plants cells or tissues. Any source of nucleic acid may be used. Preferably, the nucleic acid is genomic DNA. The nucleic acid is subjected to restriction endonuclease digestion. For example, one or more nucleic acid molecule or fragment thereof of the present invention can be used as a probe in accordance with the above-described polymorphic methods. The polymorphism obtained in this approach can then be cloned to identify the mutation at the coding region which alters the protein's structure or regulatory region of the gene which affects its expression level.

In an aspect of the present invention, one or more of the nucleic molecules of the present invention are used to determine the level (i.e., the concentration of mRNA in a sample, etc.) in a plant or pattern (i.e., the kinetics of expression, rate of decomposition, stability profile, etc.) or occurrence or absence (e.g., tissue distribution, development or environmental stage, etc.) of the expression of a protein encoded in part or whole by one or more of the nucleic acid molecule of the present invention (collectively, the “Expression Response” of a cell or tissue). As used herein, the Expression Response manifested by a cell or tissue is said to be “altered” if it differs from the Expression Response of cells or tissues of plants not exhibiting the phenotype. To determine whether a Expression Response is altered, the Expression Response manifested by the cell or tissue of the plant exhibiting the phenotype is compared with that of a similar cell or tissue sample of a plant not exhibiting the phenotype. As will be appreciated, it is not necessary to re-determine the Expression Response of the cell or tissue sample of plants not exhibiting the phenotype each time such a comparison is made; rather, the Expression Response of a particular plant may be compared with previously obtained values of normal plants. As used herein, the phenotype of the organism is any of one or more characteristics of an organism (e.g., disease resistance, pest tolerance, environmental tolerance such as tolerance to abiotic stress, male sterility, quality improvement or yield, etc.). A change in genotype or phenotype may be transient or permanent. Also as used herein, a tissue sample is any sample that comprises more than one cell. In a preferred aspect, a tissue sample comprises cells that share a common characteristic (e.g., derived from root, seed, flower, leaf, stem or pollen, etc.).

In one aspect of the present invention, an evaluation can be conducted to determine whether a particular mRNA molecule is present. One or more of the nucleic acid molecules of the present invention, preferably one or more of the EST nucleic acid molecules or fragments thereof of the present invention are utilized to detect the presence or quantity of the mRNA species. Such molecules are then incubated with cell or tissue extracts of a plant under conditions sufficient to permit nucleic acid hybridization. The detection of double-stranded probe-mRNA hybrid molecules is indicative of the presence of the mRNA; the amount of such hybrid formed is proportional to the amount of mRNA. Thus, such probes may be used to ascertain the level and extent of the mRNA production in a plant's cells or tissues. Such nucleic acid hybridization may be conducted under quantitative conditions (thereby providing a numerical value of the amount of the mRNA present). Alternatively, the assay may be conducted as a qualitative assay that indicates either that the mRNA is present, or that its level exceeds a user set, predefined value.

A principle of in situ hybridization is that a labeled, single-stranded nucleic acid probe will hybridize to a complementary strand of cellular DNA or RNA and, under the appropriate conditions, these molecules will form a stable hybrid. When nucleic acid hybridization is combined with histological techniques, specific DNA or RNA sequences can be identified within a single cell. An advantage of in situ hybridization over more conventional techniques for the detection of nucleic acids is that it allows an investigator to determine the precise spatial population (Angerer et al., Dev. Biol. 101:477-484 (1984); Angerer et al., Dev. Biol. 112:157-166 (1985); Dixon et al., EMBO J. 10: 1317-1324 (1991)). In situ hybridization may be used to measure the steady-state level of RNA accumulation. It is a sensitive technique and RNA sequences present in as few as 5-10 copies per cell can be detected (Hardin et al., J. Mol. Biol. 202:417-431 (1989)). A number of protocols have been devised for in situ hybridization, each with tissue preparation, hybridization and washing conditions (Meyerowitz, Plant Mol. Biol. Rep. 5:242-250 (1987); Cox and Goldberg, In: Plant Molecular Biology: A Practical Approach, Shaw (ed.), pp. 1-35, IRL Press, Oxford (1988); Raikhel et al., In situ RNA hybridization in plant tissues, In: Plant Molecular Biology Manual, Vol. B9: 1-32, Kluwer Academic Publisher, Dordrecht, Belgium (1989)).

In situ hybridization also allows for the localization of proteins within a tissue or cell (Wilkinson, In Situ Hybridization, Oxford University Press, Oxford (1992); Langdale, In Situ Hybridization In: The Maize Handbook, Freeling and Walbot (eds.), pp. 165-179, Springer-Verlag, New York (1994)). It is understood that one or more of the molecules of the present invention, preferably one or more of the EST nucleic acid molecules or complements thereof or fragments of either of the present invention or one or more of the antibodies of the present invention may be utilized to detect the level or pattern of a protein or mRNA thereof by in situ hybridization.

Fluorescent in situ hybridization allows the localization of a particular DNA sequence along a chromosome which is useful, among other uses, for gene mapping, following chromosomes in hybrid lines or detecting chromosomes with translocations, transversions or deletions. In situ hybridization has been used to identify chromosomes in several plant species (Griffor et al., Plant Mol. Biol. 17:101-109 (1991); Gustafson et al., Proc. Natl. Acad. Sci. (U.S.A.) 87:1899-1902 (1990); Mukai and Gill, Genome 34:448-452 (1991); Schwarzacher and Heslop-Harrison, Genome 34:317-323 (1991); Wang et al., Jpn. J. Genet. 66:313-316 (1991); Parra and Windle, Nature Genetics 5:17-21 (1993)). It is understood that the nucleic acid molecules of the present invention may be used as probes or markers to localize sequences along a chromosome.

Another method to localize the expression of a molecule is tissue printing. Tissue printing provides a way to screen, at the same time on the same membrane many tissue sections from different plants or different developmental stages. Tissue-printing procedures utilize films designed to immobilize proteins and nucleic acids. In essence, a freshly cut section of a tissue is pressed gently onto nitrocellulose paper, nylon membrane or polyvinylidene difluoride membrane. Such membranes are commercially available (e.g., Millipore, Bedford, Mass. U.S.A.). The contents of the cut cell transfer onto the membrane and the contents and are immobilized to the membrane. The immobilized contents form a latent print that can be visualized with appropriate probes. When a plant tissue print is made on nitrocellulose paper, the cell walls leave a physical print that makes the anatomy visible without further treatment (Varner and Taylor, Plant Physiol. 91:31-33 (1989)).

Tissue printing on substrate films is described by Daoust, Exp. Cell Res. 12:203-211 (1957), who detected amylase, protease, ribonuclease and deoxyribonuclease in animal tissues using starch, gelatin and agar films. These techniques can be applied to plant tissues (Yomo and Taylor, Planta 112:3543 (1973); Harris and Chrispeels, Plant Physiol. 56:292-299 (1975)). Advances in membrane technology have increased the range of applications of Daoust's tissue-printing techniques allowing (Cassab and Varner, J. Cell. Biol. 105:2581-2588 (1987)) the histochemical localization of various plant enzymes and deoxyribonuclease on nitrocellulose paper and nylon (Spruce et al., Phytochemistry 26:2901-2903 (1987); Barres et al., Neuron 5:527-544 (1990); Reid and Pont-Lezica, Tissue Printing: Tools for the Study of Anatomy, Histochemistry and Gene Expression, Academic Press, New York, N.Y. (1992); Reid et al., Plant Physiol. 93:160-165 (1990); Ye et al., Plant J. 1:175-183 (1991)).

It is understood that one or more of the molecules of the present invention, preferably one or more of the EST nucleic acid molecules or fragments thereof of the present invention or one or more of the antibodies of the present invention may be utilized to detect the presence or quantity of a protein by tissue printing.

Further it is also understood that any of the nucleic acid molecules of the present invention may be used as marker nucleic acids and or probes in connection with methods that require probes or marker nucleic acids. As used herein, a probe is an agent that is utilized to determine an attribute or feature (e.g., presence or absence, location, correlation, etc.) of a molecule, cell, tissue or plant. As used herein, a marker nucleic acid is a nucleic acid molecule that is utilized to determine an attribute or feature (e.g., presence or absence, location, correlation, etc.) or a molecule, cell, tissue or plant.

A microarray-based method for high-throughput monitoring of plant gene expression may be utilized to measure gene-specific hybridization targets. This ‘chip’-based approach involves using microarrays of nucleic acid molecules as gene-specific hybridization targets to quantitatively measure expression of the corresponding plant genes (Schena et al., Science 270:467-470 (1995); Shalon, Ph.D. Thesis, Stanford University (1996)). Every nucleotide in a large sequence can be queried at the same time. Hybridization can be used to efficiently analyze nucleotide sequences.

Several microarray methods have been described. One method compares the sequences to be analyzed by hybridization to a set of oligonucleotides representing all possible subsequences (Bains and Smith, J. Theor. Biol. 135:303-307 (1989)). A second method hybridizes the sample to an array of oligonucleotide or cDNA molecules. An array consisting of oligonucleotides complementary to subsequences of a target sequence can be used to determine the identity of a target sequence, measure its amount and detect differences between the target and a reference sequence. Nucleic acid molecules microarrays may also be screened with protein molecules or fragments thereof to determine nucleic acid molecules that specifically bind protein molecules or fragments thereof.

The microarray approach may be used with polypeptide targets (U.S. Pat. No. 5,445,934; U.S. Pat. No. 5,143,854; U.S. Pat. No. 5,079,600; U.S. Pat. No. 4,923,901). Essentially, polypeptides are synthesized on a substrate (microarray) and these polypeptides can be screened with either protein molecules or fragments thereof or nucleic acid molecules in order to screen for either protein molecules or fragments thereof or nucleic acid molecules that specifically bind the target polypeptides (Fodor et al., Science 251:767-773 (1991)). It is understood that one or more of the nucleic acid molecules or protein or fragments thereof of the present invention may be utilized in a microarray based method.

In a preferred embodiment of the present invention microarrays may be prepared that comprise nucleic acid molecules where preferably at least 10%, preferably at least 25%, more preferably at least 50% and even more preferably at least 75%, 80%, 85%, 90% or 95% of the nucleic acid molecules located on that array are selected from the group of nucleic acid molecules that specifically hybridize to one or more nucleic acid molecule having a nucleic acid sequence selected from the group of SEQ ID NO: 1 through SEQ ID NO: 294,310 or complement thereof or fragments of either.

In another preferred embodiment of the present invention microarrays may be prepared that comprise nucleic acid molecules where preferably at least 10%, preferably at least 25%, more preferably at least 50% and even more preferably at least 75%, 80%, 85%, 90% or 95% of the nucleic acid molecules located on that array are selected from the group of nucleic acid molecules having a nucleic acid sequence selected from the group of SEQ ID NO: 1 through SEQ ID NO: 294,310 or complements thereof.

In a preferred embodiment of the present invention microarrays may be prepared that comprise nucleic acid molecules where preferably at least 2%, preferably at least 5%, more preferably at least 10% and even more preferably at least 25%, 50%, 75%, 80%, 85%, 90% or 95% of the nucleic acid molecules located on that array are selected from the group of nucleic acid molecules that specifically hybridize to one or more nucleic acid molecule having a nucleic acid sequence selected from the group of sequences derived from a library where the library is selected from the group consisting of: MONN01, SATMON001, SATMON003 through SATMON014, SATMON016, SATMON017, SATMON019 through SATMON031, SATMON033, SATMON034, SATMONN01, SATMONN04 through SATMONN06, LIB36, LIB83 through LIB84, CMz029 through CMz031, CMz033 through CMz037, CMz039 through CMz042, CMz044 through CMz045, CMz047 through CMz050, SOYMON001 through SOYMON038, Soy51 through Soy56, Soy58 through Soy62, Soy65 through Soy77, LIB3054, LIB3087, and LIB3094 (Monsanto Company, St. Louis, Mo. U.S.A.).

In a preferred embodiment of the present invention microarrays may be prepared that comprise nucleic acid molecules where preferably at least 2%, preferably at least 5%, more preferably at least 10% and even more preferably at least 25%, 50%, 75%, 80%, 85%, 90% or 95% of the nucleic acid molecules located on that array are selected from the group of nucleic acid molecules having a nucleic acid sequences from the group of a sequences derived from a library where the library is selected from the group consisting of: MONN01, SATMON001, SATMON003 through SATMON014, SATMON016, SATMON017, SATMON019 through SATMON031, SATMON033, SATMON034, SATMONN01, SATMONN04 through SATMONN06, LIB36, LIB83 through LIB84, CMz029 through CMz031, CMz033 through CMz037, CMz039 through CMz042, CMz044 through CMz045, CMz047 through CMz050, SOYMON001 through SOYMON038, Soy51 through Soy56, Soy58 through Soy62, Soy65 through Soy77, LIB3054, LIB3087, and LIB3094 (Monsanto Company, St. Louis, Mo. U.S.A.).

In an even more preferred embodiment of the present invention, the microarray comprises a nucleic acid molecule and/or collection of nucleic acid molecules of the present invention where the nucleic acid molecule and/or collection of nucleic acid molecules are capable of determining or predicting a component or attribute of a biochemical process or activity where the process or activity is preferably selected from photosynthetic activity, carbohydrate metabolism, amino acid synthesis or degradation, plant hormone or other regulatory molecules, phenolic metabolism, and lipid metabolism, and more preferably selected from the group consisting of biosynthesis of tetrapyrroles, phytochrome metabolism, carbon assimilation, glycolysis and gluconeogenesis metabolism, sucrose metabolism, starch metabolism, phosphogluconate metabolism, galactomannan metabolism, raffinose metabolism, complex carbohydrate synthesis/degradation, phytic acid metabolism, methionine biosynthesis, methionine degradation, lysine metabolism, arginine metabolism, proline metabolism, glutamate/glutamine metabolism, aspartate/asparagine metabolism, cytokinin metabolism, gibberellin metabolism, ethylene metabolism, jasmonic acid synthesis metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, and fatty acid metabolism, and even more preferably selected from the group consisting of: glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

In an even more preferred embodiment of the present invention, the microarray comprises a nucleic acid molecule and/or collection of nucleic acid molecules of the present invention where the nucleic acid molecule and/or collection of nucleic acid molecules are capable of detecting or predicting a component or attribute of at least two, more preferable at least three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five or forty six biochemical processes or activities where the biochemical processes or activities are selected from the following: photosynthetic activity, carbohydrate metabolism, amino acid synthesis or degradation, plant hormone or other regulatory molecules, phenolic metabolism, lipid metabolism, biosynthesis of tetrapyrroles, phytochrome metabolism, carbon assimilation, glycolysis and gluconeogenesis metabolism, sucrose metabolism, starch metabolism, phosphogluconate metabolism, galactomannan metabolism, raffinose metabolism, complex carbohydrate synthesis/degradation, phytic acid metabolism, methionine biosynthesis, methionine degradation, lysine metabolism, arginine metabolism, proline metabolism, glutamate/glutamine, aspartate/asparagine metabolism, cytokinin metabolism, gibberellin metabolism, ethylene metabolism, jasmonic acid metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, fatty acid metabolism, glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

Site directed mutagenesis may be utilized to modify nucleic acid sequences, particularly as it is a technique that allows one or more of the amino acids encoded by a nucleic acid molecule to be altered (e.g., a threonine to be replaced by a methionine). Three basic methods for site directed mutagenesis are often employed. These are cassette mutagenesis (Wells et al., Gene 34:315-323 (1985)), primer extension (Gilliam et al., Gene 12:129-137 (1980); Zoller and Smith, Methods Enzymol. 100:468-500 (1983); Dalbadie-McFarland et al., Proc. Natl. Acad. Sci. (U.S.A.) 79:6409-6413 (1982)) and methods based upon PCR (Scharf et al., Science 233:1076-1078 (1986); Higuchi et al., Nucleic Acids Res. 16:7351-7367 (1988)). Site directed mutagenesis approaches are also described in European Patent 0 385 962; European Patent 0 359 472; and PCT Patent Application WO 93/07278.

Site directed mutagenesis strategies have been applied to plants for both in vitro as well as in vivo site directed mutagenesis (Lanz et al., J. Biol. Chem. 266:9971-9976 (1991); Kovgan and Zhdanov, Biotekhnologiya 5:148-154, No. 207160n, Chemical Abstracts 110:225 (1989); Ge et al., Proc. Natl. Acad. Sci. (U.S.A.) 86:4037-4041 (1989); Zhu et al., J. Biol. Chem. 271:18494-18498 (1996); Chu et al., Biochemistry 33:6150-6157 (1994); Small et al., EMBO J. 11: 1291-1296 (1992); Cho et al., Mol. Biotechnol. 8:13-16 (1997); Kita et al., J. Biol. Chem. 271:26529-26535 (1996), Jin et al., Mol. Microbiol. 7:555-562 (1993); Hatfield and Vierstra, J. Biol. Chem. 267:14799-14803 (1992); Zhao et al., Biochemistry 31:5093-5099 (1992)).

Any of the nucleic acid molecules of the present invention may either be modified by site directed mutagenesis or used as, for example, nucleic acid molecules that are used to target other nucleic acid molecules for modification. It is understood that mutants with more than one altered nucleotide can be constructed using techniques that practitioners are familiar with such as isolating restriction fragments and ligating such fragments into an expression vector (see, for example, Sambrook et al., Molecular Cloning: A Laboratory Manual, Cold Spring Harbor Press (1989)).

Sequence-specific DNA-binding proteins play a role in the regulation of transcription. The isolation of recombinant cDNAs encoding these proteins facilitates the biochemical analysis of their structural and functional properties. Genes encoding such DNA-binding proteins have been isolated using classical genetics (Vollbrecht et al., Nature 350:241-243 (1991)) and molecular biochemical approaches, including the screening of recombinant cDNA libraries with antibodies (Landschulz et al., Genes Dev. 2:786-800 (1988)) or DNA probes (Bodner et al., Cell 55:505-518 (1988)). In addition, an in situ screening procedure has been used and has facilitated the isolation of sequence-specific DNA-binding proteins from various plant species (Gilmartin et al., Plant Cell 4:839-849 (1992); Schindler et al., EMBO J. 11:1261-1273 (1992)). An in situ screening protocol does not require the purification of the protein of interest (Vinson et al., Genes Dev. 2:801-806 (1988); Singh et al., Cell 52:415-423 (1988)).

Two steps may be employed to characterize DNA-protein interactions. The first is to identify promoter fragments that interact with DNA-binding proteins, to titrate binding activity, to determine the specificity of binding and to determine whether a given DNA-binding activity can interact with related DNA sequences (Sambrook et al., Molecular Cloning: A Laboratory Manual, 2^{nd} edition, Cold Spring Harbor Laboratory Press, Cold Spring Harbor, N.Y. (1989)). Electrophoretic mobility-shift assay is a widely used assay. The assay provides a rapid and sensitive method for detecting DNA-binding proteins based on the observation that the mobility of a DNA fragment through a nondenaturing, low-ionic strength polyacrylamide gel is retarded upon association with a DNA-binding protein (Fried and Crother, Nucleic Acids Res. 9:6505-6525 (1981)). When one or more specific binding activities have been identified, the exact sequence of the DNA bound by the protein may be determined. Several procedures for characterizing protein/DNA-binding sites are used, including methylation and ethylation interference assays (Maxam and Gilbert, Methods Enzymol. 65:499-560 (1980); Wissman and Hillen, Methods Enymol. 208:365-379 (1991)), footprinting techniques employing DNase I (Galas and Schmitz, Nucleic Acids Res. 5:3157-3170 (1978)), 1,10-phenanthroline-copper ion methods (Sigman et al., Methods Enzymol. 208:414-433 (1991)) and hydroxyl radicals methods (Dixon et al., Methods Enzymol. 208:414-433 (1991)). It is understood that one or more of the nucleic acid molecules of the present invention may be utilized to identify a protein or fragment thereof that specifically binds to a nucleic acid molecule of the present invention. It is also understood that one or more of the protein molecules or fragments thereof of the present invention may be utilized to identify a nucleic acid molecule that specifically binds to it.

A two-hybrid system is based on the fact that many cellular functions are carried out by proteins, such as transcription factors, that interact (physically) with one another. Two-hybrid systems have been used to probe the function of new proteins (Chien et al., Proc. Natl. Acad. Sci. (U.S.A.) 88:9578-9582 (1991); Durfee et al., Genes Dev. 7:555-569 (1993); Choi et al., Cell 78:499-512 (1994); Kranz et al., Genes Dev. 8:313-327 (1994)).

Interaction mating techniques have facilitated a number of two-hybrid studies of protein-protein interaction. Interaction mating has been used to examine interactions between small sets of tens of proteins (Finley and Brent, Proc. Natl. Acad. Sci. (U.S.A.) 91:12098-12984 (1994)), larger sets of hundreds of proteins (Bendixen et al., Nucl. Acids Res. 22:1778-1779 (1994)) and to comprehensively map proteins encoded by a small genome (Bartel et al., Nature Genetics 12:72-77 (1996)). This technique utilizes proteins fused to the DNA-binding domain and proteins fused to the activation domain. They are expressed in two different haploid yeast strains of opposite mating type and the strains are mated to determine if the two proteins interact. Mating occurs when haploid yeast strains come into contact and result in the fusion of the two haploids into a diploid yeast strain. An interaction can be determined by the activation of a two-hybrid reporter gene in the diploid strain. An advantage of this technique is that it reduces the number of yeast transformations needed to test individual interactions. It is understood that the protein-protein interactions of protein or fragments thereof of the present invention may be investigated using the two-hybrid system and that any of the nucleic acid molecules of the present invention that encode such proteins or fragments thereof may be used to transform yeast in the two-hybrid system. A preferred sub-group of proteins or fragments thereof are transcription factors or fragments thereof.

(a) Plant Constructs and Plant Transformants

One or more of the nucleic acid molecules of the present invention may be used in plant transformation or transfection. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. In a preferred embodiment, the exogenous genetic material includes a nucleic acid molecule of the present invention having a sequence selected from the group consisting of SEQ ID NO: 1 through SEQ ID NO: 294,310 or complements thereof or fragments of either. In another preferred embodiment, the exogenous genetic material includes a nucleic acid molecule of the present invention selected from the group consisting of any nucleic acid molecule of the present invention.

Such genetic material may be transferred into either monocotyledons and dicotyledons including, but not limited to maize (pp. 63-69), soybean (pp. 50-60), Arabidopsis (p 45), phaseolus (pp. 47-49), peanut (pp. 49-50), alfalfa (p 60), wheat (pp. 69-71), rice (pp. 72-79), oat (pp. 80-81), sorghum (p 83), rye (p 84), tritordeum (p 84), millet (p 85), fescue (p 85), perennial ryegrass (p 86), sugarcane (p 87), cranberry (p 110), papaya (pp. 101-102), banana (p 103), banana (p 103), muskmelon (p 104), apple (p 104), cucumber (p 105), dendrobium (p 109), gladiolus (p 110), chrysanthemum (p 110), liliacea (p 111), cotton (pp 113-114), eucalyptus (p 115), sunflower (p 118), canola (p 118), turfgrass (p 121), sugarbeet (p 122), coffee (p 122) and dioscorea (p 122), (Christou, In: Particle Bombardment for Genetic Engineering of Plants, Biotechnology Intelligence Unit, Academic Press, San Diego, Calif. (1996)).

In a more preferred embodiment of the present invention, the transgenic plant comprises a nucleic molecule and/or collection of nucleic acid molecules capable of altering a biochemical process where the process or activity is preferably selected from photosynthetic activity, carbohydrate metabolism, amino acid synthesis or degradation, plant hormone or other regulatory molecules, phenolic metabolism, and lipid metabolism, and more preferably selected from the group consisting of biosynthesis of tetrapyrroles, phytochrome metabolism, carbon assimilation, glycolysis and gluconeogenesis metabolism, sucrose metabolism, starch metabolism, phosphogluconate metabolism, galactomannan metabolism, raffinose metabolism, complex carbohydrate synthesis/degradation, phytic acid metabolism, methionine biosynthesis, methionine degradation, lysine metabolism, arginine metabolism, proline metabolism, glutamate/glutamine metabolism, aspartate/asparagine metabolism, cytokinin metabolism, gibberellin metabolism, ethylene metabolism, jasmonic acid metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, and fatty acid metabolism, and even more preferably selected from the group consisting of: glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism, and in an even more preferred embodiment the transgenic plant comprises a nucleic molecule and/or collection of nucleic acid molecules selected from the group consisting of a nucleic molecule and/or collection of nucleic acid molecules which encode an mRNA or a collection of mRNA where the level, pattern, occurrence and/or absence of an mRNA and/or a collection of mRNA is a marker for a biochemical process or activity selected from the group consisting of photosynthetic activity, carbohydrate metabolism, amino acid synthesis or degradation, plant hormone or other regulatory molecules, phenolic metabolism, and lipid metabolism, and more preferably selected from the group consisting of biosynthesis of tetrapyrroles, phytochrome metabolism, carbon assimilation, glycolysis and gluconeogenesis metabolism, sucrose metabolism, starch metabolism, phosphogluconate metabolism, galactomannan metabolism, raffinose metabolism, complex carbohydrate synthesis/degradation, phytic acid metabolism, methionine biosynthesis, methionine degradation, lysine metabolism, arginine metabolism, proline metabolism, glutamate/glutamine metabolism, aspartate/asparagine metabolism, cytokinin metabolism, gibberellin metabolism, ethylene metabolism, jasmonic acid metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, and fatty acid metabolism, and even more preferably selected from the group consisting of: glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

Transfer of a nucleic acid that encodes for a protein can result in overexpression of that protein in a transformed cell or transgenic plant. One or more of the proteins or fragments thereof encoded by nucleic acid molecules of the present invention may be overexpressed in a transformed cell or transformed plant. Particularly, any of the proteins or fragments thereof may be overexpressed in a transformed cell or transgenic plant. Such overexpression may be the result of transient or stable transfer of the exogenous genetic material.

Exogenous genetic material may be transferred into a plant cell and the plant cell by the use of a DNA vector or construct designed for such a purpose. Design of such a vector is generally within the skill of the art (See, Plant Molecular Biology: A Laboratory Manual, Clark (ed.), Springier, N.Y. (1997)).

A construct or vector may include a plant promoter to express the protein or protein fragment of choice. A number of promoters which are active in plant cells have been described in the literature. These include the nopaline synthase (NOS) promoter (Ebert et al., Proc. Natl. Acad. Sci. (U.S.A.) 84:5745-5749 (1987)), the octopine synthase (OCS) promoter (which are carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987)) and the CaMV 35S promoter (Odell et al., Nature 313:810-812 (1985)), the figwort mosaic virus 35S-promoter, the light-inducible promoter from the small subunit of ribulose-1,5-bis-phosphate carboxylase (ssRUBISCO), the Adh promoter (Walker et al., Proc. Natl. Acad. Sci. (U.S.A.) 84:6624-6628 (1987)), the sucrose synthase promoter (Yang et al., Proc. Natl. Acad. Sci. (U.S.A.) 87:414-44148 (1990)), the R gene complex promoter (Chandler et al., The Plant Cell 1:1175-1183 (1989)) and the chlorophyll a/b binding protein gene promoter, etc. These promoters have been used to create DNA constructs which have been expressed in plants; see, e.g., PCT publication WO 84/02913.

Promoters which are known or are found to cause transcription of DNA in plant cells can be used in the present invention. Such promoters may be obtained from a variety of sources such as plants and plant viruses. It is preferred that the particular promoter selected should be capable of causing sufficient expression to result in the production of an effective amount of the protein to cause the desired phenotype. In addition to promoters that are known to cause transcription of DNA in plant cells, other promoters may be identified for use in the current invention by screening a plant cDNA library for genes which are selectively or preferably expressed in the target tissues or cells.

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea (Edwards et al., Proc. Natl. Acad. Sci. (U.S.A.) 87:3459-3463 (1990)), the chloroplast fructose-1,6-biphosphatase (FBPase) promoter from wheat (Lloyd et al., Mol. Gen. Genet. 225:209-216 (1991)), the nuclear photosynthetic ST-LS1 promoter from potato (Stockhaus et al., EMBO J. 8:2445-2451 (1989)), the serine/threonine kinase (PAL) promoter and the glucoamylase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine (Yamamoto et al., Plant Cell Physiol. 35:773-778 (1994)), the promoter for the Cab-1 gene from wheat (Fejes et al., Plant Mol. Biol. 15:921-932 (1990)), the promoter for the CAB-1 gene from spinach (Lubberstedt et al., Plant Physiol. 104:997-1006 (1994)), the promoter for the cab1R gene from rice (Luan et al., Plant Cell. 4:971-981 (1992)), the pyruvate, orthophosphate dikinase (PPDK) promoter from maize (Matsuoka et al., Proc. Natl. Acad. Sci. (U.S.A.) 90:9586-9590 (1993)), the promoter for the tobacco Lhcb1*2 gene (Cerdan et al., Plant Mol. Biol. 33:245-255 (1997)), the Arabidopsis thaliana SUC2 sucrose-H+ symporter promoter (Truernit et al., Planta. 196:564-570 (1995)) and the promoter for the thylakoid membrane proteins from spinach (psad, psaF, psaE, PC, FNR, atpC, atpD, cab, rbcS). Other promoters for the chlorophyll a/b-binding proteins may also be utilized in the present invention, such as the promoters for LhcB gene and PsbP gene from white mustard (Sinapis alba; Kretsch et al., Plant Mol. Biol. 28:219-229 (1995)).

For the purpose of expression in sink tissues of the plant, such as the tuber of the potato plant, the fruit of tomato, or the seed of maize, wheat, rice and barley, it is preferred that the promoters utilized in the present invention have relatively high expression in these specific tissues. A number of promoters for genes with tuber-specific or -enhanced expression are known, including the class I patatin promoter (Bevan et al., EMBO J. 8:1899-1906 (1986); Jefferson et al., Plant Mol. Biol. 14:995-1006 (1990)), the promoter for the potato tuber ADPGPP genes, both the large and small subunits, the sucrose synthase promoter (Salanoubat and Belliard, Gene. 60:47-56 (1987), Salanoubat and Belliard, Gene. 84:181-185 (1989)), the promoter for the major tuber proteins including the 22 kd protein complexes and proteinase inhibitors (Hannapel, Plant Physiol. 101:703-704 (1993)), the promoter for the granule bound starch synthase gene (GBSS) (Visser et al., Plant Mol. Biol. 17:691-699 (1991)) and other class I and II patatins promoters (Koster-Topfer et al., Mol Gen Genet. 219:390-396 (1989); Mignery et al., Gene. 62:2744 (1988)).

Other promoters can also be used to express a protein or fragment thereof of the present invention in specific tissues, such as seeds or fruits. The promoter for β-conglycinin (Chen et al., Dev. Genet. 10:112-122 (1989)) or other seed-specific promoters such as the napin and phaseolin promoters, can be used. The zeins are a group of storage proteins found in maize endosperm. Genomic clones for zein genes have been isolated (Pedersen et al., Cell 29:1015-1026 (1982)) and the promoters from these clones, including the 15 kD, 16 kD, 19 kD, 22 kD, 27 kD and γ genes, could also be used. Other promoters known to function, for example, in maize include the promoters for the following genes: waxy, Brittle, Shrunken 2, Branching enzymes I and II, starch synthases, debranching enzymes, oleosins, glutelins and sucrose synthases. A particularly preferred promoter for maize endosperm expression is the promoter for the glutelin gene from rice, more particularly the Osgt-1 promoter (Zheng et al., Mol. Cell. Biol. 13:5829-5842 (1993)). Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins and the glutenins. Examples of such promoters in rice include those promoters for the ADPGPP subunits, the granule bound and other starch synthase, the branching enzymes, the debranching enzymes, sucrose synthases and the glutelins. A particularly preferred promoter is the promoter for rice glutelin, Osgt-1. Examples of such promoters for barley include those for the ADPGPP subunits, the granule bound and other starch synthase, the branching enzymes, the debranching enzymes, sucrose synthases, the hordeins, the embryo globulins and the aleurone specific proteins.

Root specific promoters may also be used. An example of such a promoter is the promoter for the acid chitinase gene (Samac et al., Plant Mol. Biol. 25:587-596 (1994)). Expression in root tissue could also be accomplished by utilizing the root specific subdomains of the CaMV35S promoter that have been identified (Lam et al., Proc. Natl. Acad. Sci. (U.S.A.) 86:7890-7894 (1989)). Other root cell specific promoters include those reported by Conkling et al. (Conkling et al., Plant Physiol. 93:1203-1211 (1990)).

Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435; and 4,633,436. In addition, a tissue specific enhancer may be used (Fromm et al., The Plant Cell 1:977-984 (1989)).

Constructs or vectors may also include with the coding region of interest a nucleic acid sequence that acts, in whole or in part, to terminate transcription of that region. For example, such sequences have been isolated including the Tr7 3′ sequence and the NOS 3′ sequence (Ingelbrecht et al., The Plant Cell 1:671-680 (1989); Bevan et al., Nucleic Acids Res. 11:369-385 (1983)), or the like.

A vector or construct may also include regulatory elements. Examples of such include the Adh intron 1 (Callis et al., Genes and Develop. 1:1183-1200 (1987)), the sucrose synthase intron (Vasil et al., Plant Physiol. 91:1575-1579 (1989)) and the TMV omega element (Gallie et al., The Plant Cell 1:301-311 (1989)). These and other regulatory elements may be included when appropriate.

A vector or construct may also include a selectable marker. Selectable markers may also be used to select for plants or plant cells that contain the exogenous genetic material. Examples of such include, but are not limited to, a neo gene (Potrykus et al., Mol. Gen. Genet. 199:183-188 (1985)) which codes for kanamycin resistance and can be selected for using kanamycin, G418, etc.; a bar gene which codes for bialaphos resistance; a mutant EPSP synthase gene (Hinchee et al., Bio/Technology 6:915-922 (1988)) which encodes glyphosate resistance; a nitrilase gene which confers resistance to bromoxynil (Stalker et al., J. Biol. Chem. 263:6310-6314 (1988)); a mutant acetolactate synthase gene (ALS) which confers imidazolinone or sulphonylurea resistance (European Patent Application 154,204 (Sep. 11, 1985)); and a methotrexate resistant DHFR gene (Thillet et al., J. Biol. Chem. 263:12500-12508 (1988)).

A vector or construct may also include a transit peptide. Incorporation of a suitable chloroplast transit peptide may also be employed (European Patent Application Publication Number 0218571). Translational enhancers may also be incorporated as part of the vector DNA. DNA constructs could contain one or more 5′ non-translated leader sequences which may serve to enhance expression of the gene products from the resulting mRNA transcripts. Such sequences may be derived from the promoter selected to express the gene or can be specifically modified to increase translation of the mRNA. Such regions may also be obtained from viral RNAs, from suitable eukaryotic genes, or from a synthetic gene sequence. For a review of optimizing expression of transgenes, see Koziel et al., Plant Mol. Biol. 32:393-405 (1996).

A vector or construct may also include a screenable marker. Screenable markers may be used to monitor expression. Exemplary screenable markers include a β-glucuronidase or uidA gene (GUS) which encodes an enzyme for which various chromogenic substrates are known (Jefferson, Plant Mol. Biol, Rep. 5:387-405 (1987); Jefferson et al., EMBO J. 6:3901-3907 (1987)); an R-locus gene, which encodes a product that regulates the production of anthocyanin pigments (red color) in plant tissues (Dellaporta et al., Stadler Symposium 11:263-282 (1988)); a β-lactamase gene (Sutcliffe et al., Proc. Natl. Acad. Sci. (U.S.A.) 75:3737-3741 (1978)), a gene which encodes an enzyme for which various chromogenic substrates are known (e.g., PADAC, a chromogenic cephalosporin); a luciferase gene (Ow et al., Science 234:856-859 (1986)); a xylE gene (Zukowsky et al., Proc. Natl. Acad. Sci. (U.S.A.) 80:1101-1105 (1983)) which encodes a catechol dioxygenase that can convert chromogenic catechols; an α-amylase gene (Ikatu et al., Bio/Technol. 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to melanin; an α-galactosidase, which will turn a chromogenic α-galactose substrate.

Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins which are detectable, (e.g., by ELISA), small active enzymes which are detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

There are many methods for introducing transforming nucleic acid molecules into plant cells. Suitable methods are believed to include virtually any method by which nucleic acid molecules may be introduced into a cell, such as by Agrobacterium infection or direct delivery of nucleic acid molecules such as, for example, by PEG-mediated transformation, by electroporation or by acceleration of DNA coated particles, etc (Potrykus, Ann. Rev. Plant Physiol. Plant Mol. Biol. 42:205-225 (1991); Vasil, Plant Mol. Biol. 25:925-937 (1994)). For example, electroporation has been used to transform maize protoplasts (Fromm et al., Nature 312:791-793 (1986)).

Other vector systems suitable for introducing transforming DNA into a host plant cell include but are not limited to binary artificial chromosome (BIBAC) vectors (Hamilton et al., Gene 200:107-116 (1997)); and transfection with RNA viral vectors (Della-Cioppa et al., Ann. N.Y. Acad. Sci. (1996), 792 (Engineering Plants for Commercial Products and Applications), 57-61). Additional vector systems also include plant selectable YAC vectors such as those described in Mullen et al., Molecular Breeding 4:449-457 (1988).

Technology for introduction of DNA into cells is well known to those of skill in the art. Four general methods for delivering a gene into cells have been described: (1) chemical methods (Graham and van der Eb, Virology 54:536-539 (1973)); (2) physical methods such as microinjection (Capecchi, Cell 22:479-488 (1980)), electroporation (Wong and Neumann, Biochem. Biophys. Res. Commun. 107:584-587 (1982); Fromm et al., Proc. Natl. Acad. Sci. (U.S.A.) 82:5824-5828 (1985); U.S. Pat. No. 5,384,253); and the gene gun (Johnston and Tang, Methods Cell Biol. 43:353-365 (1994)); (3) viral vectors (Clapp, Clin. Perinatol. 20:155-168 (1993); Lu et al., J. Exp. Med. 178:2089-2096 (1993); Eglitis and Anderson, Biotechniques 6:608-614 (1988)); and (4) receptor-mediated mechanisms (Curiel et al., Hum. Gen. Ther. 3:147-154 (1992), Wagner et al., Proc. Natl. Acad. Sci. (USA) 89:6099-6103 (1992)).

Acceleration methods that may be used include, for example, microprojectile bombardment and the like. One example of a method for delivering transforming nucleic acid molecules to plant cells is microprojectile bombardment. This method has been reviewed by Yang and Christou (eds.), Particle Bombardment Technology for Gene Transfer, Oxford Press, Oxford, England (1994)). Non-biological particles (microprojectiles) that may be coated with nucleic acids and delivered into cells by a propelling force. Exemplary particles include those comprised of tungsten, gold, platinum and the like.

A particular advantage of microprojectile bombardment, in addition to it being an effective means of reproducibly transforming monocots, is that neither the isolation of protoplasts (Cristou et al., Plant Physiol. 87:671-674 (1988)) nor the susceptibility of Agrobacterium infection are required. An illustrative embodiment of a method for delivering DNA into maize cells by acceleration is a biolistics α-particle delivery system, which can be used to propel particles coated with DNA through a screen, such as a stainless steel or Nytex screen, onto a filter surface covered with corn cells cultured in suspension. Gordon-Kamm et al., describes the basic procedure for coating tungsten particles with DNA (Gordon-Kamm et al., Plant Cell 2:603-618 (1990)). The screen disperses the tungsten nucleic acid particles so that they are not delivered to the recipient cells in large aggregates. A particle delivery system suitable for use with the present invention is the helium acceleration PDS-1000/He gun is available from Bio-Rad Laboratories (Bio-Rad, Hercules, Calif.) (Sanford et al., Technique 3:3-16 (1991)).

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus which express the exogenous gene product 48 hours post-bombardment often range from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include the particle gun delivery of DNA containing a selectable marker and targeting of the DNA to the plastid genome through homologous recombination (Svab et al., Proc. Natl. Acad. Sci. (U.S.A.) 87:8526-8530 (1990); Svab and Maliga, Proc. Natl. Acad. Sci. (U.S.A.) 90:913-917 (1993); Staub and Maliga, EMBO J. 12:601-606 (1993); U.S. Pat. Nos. 5,451,513 and 5,545,818).

Accordingly, it is contemplated that one may wish to adjust various aspects of the bombardment parameters in small scale studies to fully optimize the conditions. One may particularly wish to adjust physical parameters such as gap distance, flight distance, tissue distance and helium pressure. One may also minimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example the methods described by Fraley et al., Bio/Technology 3:629-635 (1985) and Rogers et al., Methods Enzymol. 153:253-277 (1987). Further, the integration of the Ti-DNA is a relatively precise process resulting in few rearrangements. The region of DNA to be transferred is defined by the border sequences and intervening DNA is usually inserted into the plant genome as described (Spielmann et al., Mol. Gen. Genet. 205:34 (1986)).

Modern Agrobacterium transformation vectors are capable of replication in E. coli as well as Agrobacterium, allowing for convenient manipulations as described (Klee et al., In: Plant DNA Infectious Agents, Hohn and Schell (eds.), Springer-Verlag, New York, pp. 179-203 (1985). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes (Rogers et al., Methods Enzymol. 153:253-277 (1987)). In addition, Agrobacterium containing both armed and disarmed Ti genes can be used for the transformations. In those plant strains where Agrobacterium-mediated transformation is efficient, it is the method of choice because of the facile and defined nature of the gene transfer.

A transgenic plant formed using Agrobacterium transformation methods typically contains a single gene on one chromosome. Such transgenic plants can be referred to as being heterozygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for the gene of interest.

It is also to be understood that two different transgenic plants can also be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-transgenic plant are also contemplated, as is vegetative propagation.

Transformation of plant protoplasts can be achieved using methods based on calcium phosphate precipitation, polyethylene glycol treatment, electroporation and combinations of these treatments (See, for example, Potrykus et al., Mol. Gen. Genet. 205:193-200 (1986); Lorz et al., Mol. Gen. Genet. 199:178 (1985); Fromm et al., Nature 319:791 (1986); Uchimiya et al., Mol. Gen. Genet. 204:204 (1986); Marcotte et al., Nature 335:454-457 (1988)).

Application of these systems to different plant strains depends upon the ability to regenerate that particular plant strain from protoplasts. Illustrative methods for the regeneration of cereals from protoplasts are described (Fujimura et al., Plant Tissue Culture Letters 2:74 (1985); Toriyama et al., Theor Appl. Genet. 205:34 (1986); Yamada et al., Plant Cell Rep. 4:85 (1986); Abdullah et al., Biotechnology 4:1087 (1986)).

To transform plant strains that cannot be successfully regenerated from protoplasts, other ways to introduce DNA into intact cells or tissues can be utilized. For example, regeneration of cereals from immature embryos or explants can be effected as described (Vasil, Biotechnology 6:397 (1988)). In addition, “particle gun” or high-velocity microprojectile technology can be utilized (Vasil et al., Bio/Technology 10:667 (1992)).

Using the latter technology, DNA is carried through the cell wall and into the cytoplasm on the surface of small metal particles as described (Klein et al., Nature 328:70 (1987); Klein et al., Proc. Natl. Acad. Sci. (U.S.A.) 85:8502-8505 (1988); McCabe et al., Bio/Technology 6:923 (1988)). The metal particles penetrate through several layers of cells and thus allow the transformation of cells within tissue explants.

Other methods of cell transformation can also be used and include but are not limited to introduction of DNA into plants by direct DNA transfer into pollen (Hess et al., Intern Rev. Cytol. 107:367 (1987); Luo et al., Plant Mol. Biol. Reporter 6:165 (1988)), by direct injection of DNA into reproductive organs of a plant (Pena et al., Nature 325:274 (1987)), or by direct injection of DNA into the cells of immature embryos followed by the rehydration of desiccated embryos (Neuhaus et al., Theor. Appl. Genet. 75:30 (1987)).

The regeneration, development and cultivation of plants from single plant protoplast transformants or from various transformed explants is well known in the art (Weissbach and Weissbach, In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens and obtaining transgenic plants have been published for cotton (U.S. Pat. No. 5,004,863; U.S. Pat. No. 5,159,135; U.S. Pat. No. 5,518,908); soybean (U.S. Pat. No. 5,569,834; U.S. Pat. No. 5,416,011; McCabe et al., Biotechnology 6:923 (1988); Christou et al., Plant Physiol. 87:671-674 (1988)); Brassica (U.S. Pat. No. 5,463,174); peanut (Cheng et al., Plant Cell Rep. 15:653-657 (1996), McKently et al., Plant Cell Rep. 14:699-703 (1995)); papaya; and pea (Grant et al., Plant Cell Rep. 15:254-258 (1995)).

Transformation of monocotyledons using electroporation, particle bombardment and Agrobacterium have also been reported. Transformation and plant regeneration have been achieved in asparagus (Bytebier et al., Proc. Natl. Acad. Sci. (USA) 84:5354 (1987)); barley (Wan and Lemaux, Plant Physiol 104:37 (1994)); maize (Rhodes et al., Science 240:204 (1988); Gordon-Kamm et al., Plant Cell 2:603-618 (1990); Fromm et al., Bio/Technology 8:833 (1990); Koziel et al., Bio/Technology 11:194 (1993); Armstrong et al., Crop Science 35:550-557 (1995); oat (Somers et al., Bio/Technology 10:1589 (1992)); orchard grass (Horn et al., Plant Cell Rep. 7:469 (1988)); rice (Toriyama et al., Theor Appl. Genet. 205:34 (1986); Part et al., Plant Mol. Biol. 32:1135-1148 (1996); Abedinia et al., Aust. J. Plant Physiol. 24:133-141 (1997); Zhang and Wu, Theor. Appl. Genet. 76:835 (1988); Zhang et al., Plant Cell Rep. 7:379 (1988); Battraw and Hall, Plant Sci. 86:191-202 (1992); Christou et al., Bio/Technology 9:957 (1991)); rye (De la Pena et al., Nature 325:274 (1987)); sugarcane (Bower and Birch, Plant J. 2:409 (1992)); tall fescue (Wang et al., Bio/Technology 10:691 (1992)) and wheat (Vasil et al., Bio/Technology 10:667 (1992); U.S. Pat. No. 5,631,152.)

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)). Transient expression systems may be used to functionally dissect gene constructs (see generally, Mailga et al., Methods in Plant Molecular Biology, Cold Spring Harbor Press (1995)).

Any of the nucleic acid molecules of the present invention may be introduced into a plant cell in a permanent or transient manner in combination with other genetic elements such as vectors, promoters, enhancers, etc. Further, any of the nucleic acid molecules of the present invention may be introduced into a plant cell in a manner that allows for overexpression of the protein or fragment thereof encoded by the nucleic acid molecule.

Cosuppression is the reduction in expression levels, usually at the level of RNA, of a particular endogenous gene or gene family by the expression of a homologous sense construct that is capable of transcribing mRNA of the same strandedness as the transcript of the endogenous gene (Napoli et al., Plant Cell 2:279-289 (1990); van der Krol et al., Plant Cell 2:291-299 (1990)). Cosuppression may result from stable transformation with a single copy nucleic acid molecule that is homologous to a nucleic acid sequence found with the cell (Prolls and Meyer, Plant J. 2:465-475 (1992)) or with multiple copies of a nucleic acid molecule that is homologous to a nucleic acid sequence found with the cell (Mittlesten et al., Mol. Gen. Genet. 244:325-330 (1994)). Genes, even though different, linked to homologous promoters may result in the cosuppression of the linked genes (Vaucheret, C.R. Acad. Sci. III 316:1471-1483 (1993)).

This technique has, for example, been applied to generate white flowers from red petunia and tomatoes that do not ripen on the vine. Up to 50% of petunia transformants that contained a sense copy of the glucoamylase (CHS) gene produced white flowers or floral sectors; this was as a result of the post-transcriptional loss of mRNA encoding CHS (Flavell, Proc. Natl. Acad. Sci. (U.S.A.) 91:3490-3496 (1994)); van Blokland et al., Plant J. 6:861-877 (1994)). Cosuppression may require the coordinate transcription of the transgene and the endogenous gene and can be reset by a developmental control mechanism (Jorgensen, Trends Biotechnol. 8:340-344 (1990); Meins and Kunz, In: Gene Inactivation and Homologous Recombination in Plants, Paszkowski (ed.), pp. 335-348, Kluwer Academic, Netherlands (1994)).

It is understood that one or more of the nucleic acids of the present invention may be introduced into a plant cell and transcribed using an appropriate promoter with such transcription resulting in the cosuppression of an endogenous protein.

Antisense approaches are a way of preventing or reducing gene function by targeting the genetic material (Mol et al., FEBS Lett. 268:427-430 (1990)). The objective of the antisense approach is to use a sequence complementary to the target gene to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes (Hiatt et al., In: Genetic Engineering, Setlow (ed.), Vol. 11, New York: Plenum 49-63 (1989)).

The principle of regulation by antisense RNA is that RNA that is complementary to the target mRNA is introduced into cells, resulting in specific RNA:RNA duplexes being formed by base pairing between the antisense substrate and the target mRNA (Green et al., Annu. Rev. Biochem. 55:569-597 (1986)). Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression (Takayama and Inouye, Crit. Rev. Biochem. Mol. Biol. 25:155-184 (1990)). An antisense vector is constructed by standard procedures and introduced into cells by transformation, transfection, electroporation, microinjection, infection, etc. The type of transformation and choice of vector will determine whether expression is transient or stable. The promoter used for the antisense gene may influence the level, timing, tissue, specificity, or inducibility of the antisense inhibition.

It is understood that the activity of a protein in a plant cell may be reduced or depressed by growing a transformed plant cell containing a nucleic acid molecule whose non-transcribed strand encodes a protein or fragment thereof.

Antibodies have been expressed in plants (Hiatt et al., Nature 342:76-78 (1989); Conrad and Fielder, Plant Mol. Biol. 26:1023-1030 (1994)). Cytoplasmic expression of a scFv (single-chain Fv antibodies) has been reported to delay infection by artichoke mottled crinkle virus. Transgenic plants that express antibodies directed against endogenous proteins may exhibit a physiological effect (Philips et al., EMBO J. 16:4489-4496 (1997); Marion-Poll, Trends in Plant Science 2:447-448 (1997)). For example, expressed anti-abscisic antibodies have been reported to result in a general perturbation of seed development (Philips et al., EMBO J. 16:4489-4496 (1997)).

Antibodies that are catalytic may also be expressed in plants (abzymes). The principle behind abzymes is that since antibodies may be raised against many molecules, this recognition ability can be directed toward generating antibodies that bind transition states to force a chemical reaction forward (Persidas, Nature Biotechnology 15:1313-1315 (1997); Baca et al., Ann. Rev. Biophys. Biomol. Struct. 26:461-493 (1997)). The catalytic abilities of abzymes may be enhanced by site directed mutagenesis. Examples of abzymes are, for example, set forth in U.S. Pat. No. 5,658,753; U.S. Pat. No. 5,632,990; U.S. Pat. No. 5,631,137; U.S. Pat. No. 5,602,015; U.S. Pat. No. 5,559,538; U.S. Pat. No. 5,576,174; U.S. Pat. No. 5,500,358; U.S. Pat. No. 5,318,897; U.S. Pat. No. 5,298,409; U.S. Pat. No. 5,258,289 and U.S. Pat. No. 5,194,585.

It is understood that any of the antibodies of the present invention may be expressed in plants and that such expression can result in a physiological effect. It is also understood that any of the expressed antibodies may be catalytic.

(b) Fungal Constructs and Fungal Transformants

The present invention also relates to a fungal recombinant vector comprising exogenous genetic material. The present invention also relates to a fungal cell comprising a fungal recombinant vector. The present invention also relates to methods for obtaining a recombinant fungal host cell comprising introducing into a fungal host cell exogenous genetic material.

Exogenous genetic material may be transferred into a fungal cell. In a preferred embodiment the exogenous genetic material includes a nucleic acid molecule of the present invention having a sequence selected from the group consisting of SEQ ID NO: 1 through SEQ ID NO: 294,310 or complements thereof or fragments of either. The fungal recombinant vector may be any vector which can be conveniently subjected to recombinant DNA procedures. The choice of a vector will typically depend on the compatibility of the vector with the fungal host cell into which the vector is to be introduced. The vector may be a linear or a closed circular plasmid. The vector system may be a single vector or plasmid or two or more vectors or plasmids which together contain the total DNA to be introduced into the genome of the fungal host.

The fungal vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the fungal cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the fungal host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the fungal host cell and, furthermore, may be non-encoding or encoding sequences.

For autonomous replication, the vector may further comprise an origin of replication enabling the vector to replicate autonomously in the host cell in question. Examples of origin of replications for use in a yeast host cell are the 2 micron origin of replication and the combination of CEN3 and ARS 1. Any origin of replication may be used which is compatible with the fungal host cell of choice.

The fungal vectors of the present invention preferably contain one or more selectable markers which permit easy selection of transformed cells. A selectable marker is a gene the product of which provides, for example biocide or viral resistance, resistance to heavy metals, prototrophy to auxotrophs and the like. The selectable marker may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase) and sC (sulfate adenyltransferase) and trpC (anthranilate synthase). Preferred for use in an Aspergillus cell are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243. A nucleic acid sequence of the present invention may be operably linked to a suitable promoter sequence. The promoter sequence is a nucleic acid sequence which is recognized by the fungal host cell for expression of the nucleic acid sequence. The promoter sequence contains transcription and translation control sequences which mediate the expression of the protein or fragment thereof.

A promoter may be any nucleic acid sequence which shows transcriptional activity in the fungal host cell of choice and may be obtained from genes encoding polypeptides either homologous or heterologous to the host cell. Examples of suitable promoters for directing the transcription of a nucleic acid construct of the invention in a filamentous fungal host are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase and hybrids thereof. In a yeast host, a useful promoter is the Saccharomyces cerevisiae enolase (eno-1) promoter. Particularly preferred promoters are the TAKA amylase, NA2-tpi (a hybrid of the promoters from the genes encoding Aspergillus niger neutral alpha-amylase and Aspergillus oryzae triose phosphate isomerase) and glaA promoters.

A protein or fragment thereof encoding nucleic acid molecule of the present invention may also be operably linked to a terminator sequence at its 3′ terminus. The terminator sequence may be native to the nucleic acid sequence encoding the protein or fragment thereof or may be obtained from foreign sources. Any terminator which is functional in the fungal host cell of choice may be used in the present invention, but particularly preferred terminators are obtained from the genes encoding Aspergillus oryzae TAKA amylase, Aspergillus niger glucoamylase, Aspergillus nidulans anthranilate synthase, Aspergillus niger alpha-glucosidase and Saccharomyces cerevisiae enolase.

A protein or fragment thereof encoding nucleic acid molecule of the present invention may also be operably linked to a suitable leader sequence. A leader sequence is a nontranslated region of a mRNA which is important for translation by the fungal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the fungal host cell of choice may be used in the present invention, but particularly preferred leaders are obtained from the genes encoding Aspergillus oryzae TAKA amylase and Aspergillus oryzae triose phosphate isomerase.

A polyadenylation sequence may also be operably linked to the 3′ terminus of the nucleic acid sequence of the present invention. The polyadenylation sequence is a sequence which when transcribed is recognized by the fungal host to add polyadenosine residues to transcribed mRNA. The polyadenylation sequence may be native to the nucleic acid sequence encoding the protein or fragment thereof or may be obtained from foreign sources. Any polyadenylation sequence which is functional in the fungal host of choice may be used in the present invention, but particularly preferred polyadenylation sequences are obtained from the genes encoding Aspergillus oryzae TAKA amylase, Aspergillus niger glucoamylase, Aspergillus nidulans anthranilate synthase and Aspergillus niger alpha-glucosidase.

To avoid the necessity of disrupting the cell to obtain the protein or fragment thereof and to minimize the amount of possible degradation of the expressed protein or fragment thereof within the cell, it is preferred that expression of the protein or fragment thereof gives rise to a product secreted outside the cell. To this end, a protein or fragment thereof of the present invention may be linked to a signal peptide linked to the amino terminus of the protein or fragment thereof. A signal peptide is an amino acid sequence which permits the secretion of the protein or fragment thereof from the fungal host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in translation reading frame with the segment of the coding region which encodes the secreted protein or fragment thereof. Alternatively, the 5′ end of the coding sequence may contain a signal peptide coding region which is foreign to that portion of the coding sequence which encodes the secreted protein or fragment thereof. The foreign signal peptide may be required where the coding sequence does not normally contain a signal peptide coding region. Alternatively, the foreign signal peptide may simply replace the natural signal peptide to obtain enhanced secretion of the desired protein or fragment thereof. The foreign signal peptide coding region may be obtained from a glucoamylase or an amylase gene from an Aspergillus species, a lipase or proteinase gene from Rhizomucor miehei, the gene for the alpha-factor from Saccharomyces cerevisiae, or the calf preprochymosin gene. An effective signal peptide for fungal host cells is the Aspergillus oryzae TAKA amylase signal, Aspergillus niger neutral amylase signal, the Rhizomucor miehei aspartic proteinase signal, the Humicola lanuginosus cellulase signal, or the Rhizomucor miehei lipase signal. However, any signal peptide capable of permitting secretion of the protein or fragment thereof in a fungal host of choice may be used in the present invention.

A protein or fragment thereof encoding nucleic acid molecule of the present invention may also be linked to a propeptide coding region. A propeptide is an amino acid sequence found at the amino terminus of aproprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme. The propeptide coding region may be native to the protein or fragment thereof or may be obtained from foreign sources. The foreign propeptide coding region may be obtained from the Saccharomyces cerevisiae alpha-factor gene or Myceliophthora thermophila laccase gene (WO 95/33836).

The procedures used to ligate the elements described above to construct the recombinant expression vector of the present invention are well known to one skilled in the art (see, for example, Sambrook et al., Molecular Cloning, A Laboratory Manual, 2nd ed., Cold Spring Harbor, N.Y., (1989)).

The present invention also relates to recombinant fungal host cells produced by the methods of the present invention which are advantageously used with the recombinant vector of the present invention. The cell is preferably transformed with a vector comprising a nucleic acid sequence of the invention followed by integration of the vector into the host chromosome. The choice of fungal host cells will to a large extent depend upon the gene encoding the protein or fragment thereof and its source. The fungal host cell may, for example, be a yeast cell or a filamentous fungal cell.

“Yeast” as used herein includes Ascosporogenous yeast (Endomycetales), Basidiosporogenous yeast and yeast belonging to the Fungi Imperfecti (Blastomycetes). The Ascosporogenous yeasts are divided into the families Spermophthoraceae and Saccharomycetaceae. The latter is comprised of four subfamilies, Schizosaccharomycoideae (for example, genus Schizosaccharomyces), Nadsonioideae, Lipomycoideae and Saccharomycoideae (for example, genera Pichia, Kluyveromyces and Saccharomyces). The Basidiosporogenous yeasts include the genera Leucosporidim, Rhodosporidium, Sporidiobolus, Filobasidium and Filobasidiella. Yeast belonging to the Fungi Imperfecti are divided into two families, Sporobolomycetaceae (for example, genera Sorobolomyces and Bullera) and Cryptococcaceae (for example, genus Candida). Since the classification of yeast may change in the future, for the purposes of this invention, yeast shall be defined as described in Biology and Activities of Yeast (Skinner et al., Soc. App. Bacteriol. Symposium Series No. 9, (1980)). The biology of yeast and manipulation of yeast genetics are well known in the art (see, for example, Biochemistry and Genetics of Yeast, Bacil et al. (ed.), 2nd edition, 1987; The Yeasts, Rose and Harrison (eds.), 2nd ed., (1987); and The Molecular Biology of the Yeast Saccharomyces, Strathern et al. (eds.), (1981)).

“Fungi” as used herein includes the phyla Ascomycota, Basidiomycota, Chytridiomycota and Zygomycota (as defined by Hawksworth et al., In: Ainsworth and Bisby's Dictionary of The Fungi, 8th edition, 1995, CAB International, University Press, Cambridge, UK) as well as the Oomycota (as cited in Hawksworth et al., In: Ainsworth and Bisby's Dictionary of The Fungi, 8th edition, 1995, CAB International, University Press, Cambridge, UK) and all mitosporic fungi (Hawksworth et al., In: Ainsworth and Bisby's Dictionary of The Fungi, 8th edition, 1995, CAB International, University Press, Cambridge, UK). Representative groups of Ascomycota include, for example, Neurospora, Eupenicillium (=Penicillium), Emericella (=Aspergillus), Eurotiun (=Aspergillus) and the true yeasts listed above. Examples of Basidiomycota include mushrooms, rusts and smuts. Representative groups of Chytridiomycota include, for example, Allomyces, Blastocladiella, Coelomomyces and aquatic fungi. Representative groups of Oomycota include, for example, Saprolegniomycetous aquatic fungi (water molds) such as Achlya. Examples of mitosporic fungi include Aspergillus, Penicilliun, Candida and Alternaria. Representative groups of Zygomycota include, for example, Rhizopus and Mucor.

“Filamentous fungi” include all filamentous forms of the subdivision Eumycota and Oomycota (as defined by Hawksworth et al., In: Ainsworth and Bisby's Dictionary of The Fungi, 8th edition, 1995, CAB International, University Press, Cambridge, UK). The filamentous fungi are characterized by a vegetative mycelium composed of chitin, cellulose, glucan, chitosan, mannan and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In one embodiment, the fungal host cell is a yeast cell. In a preferred embodiment, the yeast host cell is a cell of the species of Candida, Kluyveromyces, Saccharomyces, Schizosaccharomyces, Pichia and Yarrowia. In a preferred embodiment, the yeast host cell is a Saccharomyces cerevisiae cell, a Saccharomyces carlsbergensis, Saccharomyces diastaticus cell, a Saccharomyces douglasii cell, a Saccharomyces kluyveri cell, a Saccharomyces norbensis cell, or a Saccharomyces oviformis cell. In another preferred embodiment, the yeast host cell is a Kluyveromyces lactis cell. In another preferred embodiment, the yeast host cell is a Yarrowia lipolytica cell.

In another embodiment, the fungal host cell is a filamentous fungal cell. In a preferred embodiment, the filamentous fungal host cell is a cell of the species of, but not limited to, Acremonium, Aspergillus, Fusarium, Humicola, Myceliophthora, Mucor, Neurospora, Penicillium, Thielavia, Tolypocladium and Trichoderma. In a preferred embodiment, the filamentous fungal host cell is an Aspergillus cell. In another preferred embodiment, the filamentous fungal host cell is an Acremonium cell. In another preferred embodiment, the filamentous fungal host cell is a Fusarium cell. In another preferred embodiment, the filamentous fungal host cell is a Humicola cell. In another preferred embodiment, the filamentous fungal host cell is a Myceliophthora cell. In another even preferred embodiment, the filamentous fungal host cell is a Mucor cell. In another preferred embodiment, the filamentous fungal host cell is a Neurospora cell. In another preferred embodiment, the filamentous fungal host cell is a Penicillium cell. In another preferred embodiment, the filamentous fungal host cell is a Thielavia cell. In another preferred embodiment, the filamentous fungal host cell is a Tolypocladiun cell. In another preferred embodiment, the filamentous fungal host cell is a Trichoderma cell. In a preferred embodiment, the filamentous fungal host cell is an Aspergillus oryzae cell, an Aspergillus niger cell, an Aspergillus foetidus cell, or an Aspergillus japonicus cell. In another preferred embodiment, the filamentous fungal host cell is a Fusarium oxysporum cell or a Fusarium graminearum cell. In another preferred embodiment, the filamentous fungal host cell is a Humicola insolens cell or a Humicola lanuginosus cell. In another preferred embodiment, the filamentous fungal host cell is a Myceliophthora thermophila cell. In a most preferred embodiment, the filamentous fungal host cell is a Mucor miehei cell. In a most preferred embodiment, the filamentous fungal host cell is a Neurospora crassa cell. In a most preferred embodiment, the filamentous fungal host cell is a Penicillium purpurogenum cell. In another most preferred embodiment, the filamentous fungal host cell is a Thielavia terrestris cell. In another most preferred embodiment, the Trichoderma cell is a Trichoderma reesei cell, a Trichoderma viride cell, a Trichoderma longibrachiatum cell, a Trichoderma harzianum cell, or a Trichoderma koningii cell. In a preferred embodiment, the fungal host cell is selected from an A. nidulans cell, an A. niger cell, an A. oryzae cell and an A. sojae cell. In a further preferred embodiment, the fungal host cell is an A. nidulans cell.

The recombinant fungal host cells of the present invention may further comprise one or more sequences which encode one or more factors that are advantageous in the expression of the protein or fragment thereof, for example, an activator (e.g., a trans-acting factor), a chaperone and a processing protease. The nucleic acids encoding one or more of these factors are preferably not operably linked to the nucleic acid encoding the protein or fragment thereof. An activator is a protein which activates transcription of a nucleic acid sequence encoding a polypeptide (Kudla et al., EMBO 9:1355-1364(1990); Jarai and Buxton, Current Genetics 26:2238-244(1994); Verdier, Yeast 6:271-297(1990)). The nucleic acid sequence encoding an activator may be obtained from the genes encoding Saccharomyces cerevisiae heme activator protein 1 (hap1), Saccharomyces cerevisiae galactose metabolizing protein 4 (gal4) and Aspergillus nidulans ammonia regulation protein (areA). For further examples, see Verdier, Yeast 6:271-297 (1990); MacKenzie et al., Journal of Gen. Microbiol. 139:2295-2307 (1993)). A chaperone is a protein which assists another protein in folding properly (Hartl et al., TIBS 19:20-25 (1994); Bergeron et al., TIBS 19:124-128 (1994); Demolder et al., J. Biotechnology 32:179-189 (1994); Craig, Science 260:1902-1903(1993); Gething and Sambrook, Nature 355:3345 (1992); Puig and Gilbert, J. Biol. Chem. 269:7764-7771 (1994); Wang and Tsou, FASEB Journal 7:1515-11157 (1993); Robinson et al., Bio/technology 1:381-384 (1994)). The nucleic acid sequence encoding a chaperone may be obtained from the genes encoding Aspergillus oryzae protein disulphide isomerase, Saccharomyces cerevisiae calnexin, Saccharomyces cerevisiae BiP/GRP78 and Saccharomyces cerevisiae Hsp70. For further examples, see Gething and Sambrook, Nature 355:33-45 (1992); Hartl et al., TIBS 19:20-25 (1994). A processing protease is a protease that cleaves a propeptide to generate a mature biochemically active polypeptide (Enderlin and Ogrydziak, Yeast 10:67-79 (1994); Fuller et al., Proc. Natl. Acad. Sci. (U.S.A.) 86:1434-1438 (1989); Julius et al., Cell 37:1075-1089 (1984); Julius et al., Cell 32:839-852 (1983)). The nucleic acid sequence encoding a processing protease may be obtained from the genes encoding Aspergillus niger Kex2, Saccharomyces cerevisiae dipeptidylaminopeptidase, Saccharomyces cerevisiae Kex2 and Yarrowia lipolytica dibasic processing endoprotease (xpr6). Any factor that is functional in the fungal host cell of choice may be used in the present invention.

Fungal cells may be transformed by a process involving protoplast formation, transformation of the protoplasts and regeneration of the cell wall in a manner known per se. Suitable procedures for transformation of Aspergillus host cells are described in EP 238 023 and Yelton et al., Proc. Natl. Acad. Sci. (U.S.A.) 81:1470-1474 (1984). A suitable method of transforming Fusarium species is described by Malardier et al., Gene 78:147-156 (1989). Yeast may be transformed using the procedures described by Becker and Guarente, In: Abelson and Simon, (eds.), Guide to Yeast Genetics and Molecular Biology, Methods Enzymol. Volume 194, pp. 182-187, Academic Press, Inc., New York; Ito et al., J. Bacteriology 153:163 (1983); Hinnen et al., Proc. Natl. Acad. Sci. (U.S.A.) 75:1920 (1978).

The present invention also relates to methods of producing the protein or fragment thereof comprising culturing the recombinant fungal host cells under conditions conducive for expression of the protein or fragment thereof. The fungal cells of the present invention are cultivated in a nutrient medium suitable for production of the protein or fragment thereof using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the protein or fragment thereof to be expressed and/or isolated. The cultivation takes place in a suitable nutrient medium comprising carbon and nitrogen sources and inorganic salts, using procedures known in the art (see, e.g., Bennett and LaSure (eds.), More Gene Manipulations in Fungi, Academic Press, Calif., (1991)). Suitable media are available from commercial suppliers or may be prepared according to published compositions (e.g., in catalogues of the American Type Culture Collection, Manassas, Va.). If the protein or fragment thereof is secreted into the nutrient medium, a protein or fragment thereof can be recovered directly from the medium. If the protein or fragment thereof is not secreted, it is recovered from cell lysates.

The expressed protein or fragment thereof may be detected using methods known in the art that are specific for the particular protein or fragment. These detection methods may include the use of specific antibodies, formation of an enzyme product, or disappearance of an enzyme substrate. For example, if the protein or fragment thereof has enzymatic activity, an enzyme assay may be used. Alternatively, if polyclonal or monoclonal antibodies specific to the protein or fragment thereof are available, immunoassays may be employed using the antibodies to the protein or fragment thereof. The techniques of enzyme assay and immunoassay are well known to those skilled in the art.

The resulting protein or fragment thereof may be recovered by methods known in the arts. For example, the protein or fragment thereof may be recovered from the nutrient medium by conventional procedures including, but not limited to, centrifugation, filtration, extraction, spray-drying, evaporation, or precipitation. The recovered protein or fragment thereof may then be further purified by a variety of chromatographic procedures, e.g., ion exchange chromatography, gel filtration chromatography, affinity chromatography, or the like.

(c) Mammalian Constructs and Transformed Mammalian Cells

The present invention also relates to methods for obtaining a recombinant mammalian host cell, comprising introducing into a mammalian host cell exogenous genetic material. The present invention also relates to a mammalian cell comprising a mammalian recombinant vector. The present invention also relates to methods for obtaining a recombinant mammalian host cell, comprising introducing into a mammalian cell exogenous genetic material. In a preferred embodiment the exogenous genetic material includes a nucleic acid molecule of the present invention having a sequence selected from the group consisting of SEQ ID NO: 1 through SEQ ID NO: 294,310 or complements thereof or fragments of either.

Mammalian cell lines available as hosts for expression are known in the art and include many immortalized cell lines available from the American Type Culture Collection (ATCC, Manassas, Va.), such as HeLa cells, Chinese hamster ovary (CHO) cells, baby hamster kidney (BHK) cells and a number of other cell lines. Suitable promoters for mammalian cells are also known in the art and include viral promoters such as that from Simian Virus 40 (SV40) (Fiers et al., Nature 273:113 (1978)), Rous sarcoma virus (RSV), adenovirus (ADV) and bovine papilloma virus (BPV). Mammalian cells may also require terminator sequences and poly-A addition sequences. Enhancer sequences which increase expression may also be included and sequences which promote amplification of the gene may also be desirable (for example methotrexate resistance genes).

Vectors suitable for replication in mammalian cells may include viral replicons, or sequences which insure integration of the appropriate sequences encoding HCV epitopes into the host genome. For example, another vector used to express foreign DNA is vaccinia virus. In this case, for example, a nucleic acid molecule encoding a protein or fragment thereof is inserted into the vaccinia genome. Techniques for the insertion of foreign DNA into the vaccinia virus genome are known in the art and may utilize, for example, homologous recombination. Such heterologous DNA is generally inserted into a gene which is non-essential to the virus, for example, the thymidine kinase gene (tk), which also provides a selectable marker. Plasmid vectors that greatly facilitate the construction of recombinant viruses have been described (see, for example, Mackett et al, J. Virol. 49:857 (1984); Chakrabarti et al., Mol. Cell. Biol. 5:3403 (1985); Moss, In: Gene Transfer Vectors For Mammalian Cells (Miller and Calos, eds., Cold Spring Harbor Laboratory, N.Y., p. 10, (1987)). Expression of the HCV polypeptide then occurs in cells or animals which are infected with the live recombinant vaccinia virus.

The sequence to be integrated into the mammalian sequence may be introduced into the primary host by any convenient means, which includes calcium precipitated DNA, spheroplast fusion, transformation, electroporation, biolistics, lipofection, microinjection, or other convenient means. Where an amplifiable gene is being employed, the amplifiable gene may serve as the selection marker for selecting hosts into which the amplifiable gene has been introduced. Alternatively, one may include with the amplifiable gene another marker, such as a drug resistance marker, e.g., neomycin resistance (G418 in mammalian cells), hygromycin in resistance, etc., or an auxotrophy marker (HIS3, TRP1, LEU2, URA3, ADE2, LYS2, etc.) for use in yeast cells.

Depending upon the nature of the modification and associated targeting construct, various techniques may be employed for identifying targeted integration. Conveniently, the DNA may be digested with one or more restriction enzymes and the fragments probed with an appropriate DNA fragment which will identify the properly sized restriction fragment associated with integration.

One may use different promoter sequences, enhancer sequences, or other sequence which will allow for enhanced levels of expression in the expression host. Thus, one may combine an enhancer from one source, a promoter region from another source, a 5′-noncoding region upstream from the initiation methionine from the same or different source as the other sequences and the like. One may provide for an intron in the non-coding region with appropriate splice sites or for an alternative 3′-untranslated sequence or polyadenylation site. Depending upon the particular purpose of the modification, any of these sequences may be introduced, as desired.

Where selection is intended, the sequence to be integrated will have with it a marker gene, which allows for selection. The marker gene may conveniently be downstream from the target gene and may include resistance to a cytotoxic agent, e.g., antibiotics, heavy metals, or the like, resistance or susceptibility to HAT, gancyclovir, etc., complementation to an auxotrophic host, particularly by using an auxotrophic yeast as the host for the subject manipulations, or the like. The marker gene may also be on a separate DNA molecule, particularly with primary mammalian cells. Alternatively, one may screen the various transformants, due to the high efficiency of recombination in yeast, by using hybridization analysis, PCR, sequencing, or the like.

For homologous recombination, constructs can be prepared where the amplifiable gene will be flanked, normally on both sides with DNA homologous with the DNA of the target region. Depending upon the nature of the integrating DNA and the purpose of the integration, the homologous DNA will generally be within 100 kb, usually 50 kb, preferably about 25 kb, of the transcribed region of the target gene, more preferably within 2 kb of the target gene. Where modeling of the gene is intended, homology will usually be present proximal to the site of the mutation. The homologous DNA may include the 5′-upstream region outside of the transcriptional regulatory region or comprising any enhancer sequences, transcriptional initiation sequences, adjacent sequences, or the like. The homologous region may include a portion of the coding region, where the coding region may be comprised only of an open reading frame or combination of exons and introns. The homologous region may comprise all or a portion of an intron, where all or a portion of one or more exons may also be present. Alternatively, the homologous region may comprise the 3′-region, so as to comprise all or a portion of the transcriptional termination region, or the region 3′ of this region. The homologous regions may extend over all or a portion of the target gene or be outside the target gene comprising all or a portion of the transcriptional regulatory regions and/or the structural gene.

The integrating constructs may be prepared in accordance with conventional ways, where sequences may be synthesized, isolated from natural sources, manipulated, cloned, ligated, subjected to in vitro mutagenesis, primer repair, or the like. At various stages, the joined sequences may be cloned and analyzed by restriction analysis, sequencing, or the like. Usually during the preparation of a construct where various fragments are joined, the fragments, intermediate constructs and constructs will be carried on a cloning vector comprising a replication system functional in a prokaryotic host, e.g., E. coli and a marker for selection, e.g., biocide resistance, complementation to an auxotrophic host, etc. Other functional sequences may also be present, such as polylinkers, for ease of introduction and excision of the construct or portions thereof, or the like. A large number of cloning vectors are available such as pBR322, the pUC series, etc. These constructs may then be used for integration into the primary mammalian host.

In the case of the primary mammalian host, a replicating vector may be used. Usually, such vector will have a viral replication system, such as SV40, bovine papilloma virus, adenovirus, or the like. The linear DNA sequence vector may also have a selectable marker for identifying transfected cells. Selectable markers include the neo gene, allowing for selection with G418, the herpes tk gene for selection with HAT medium, the gpt gene with mycophenolic acid, complementation of an auxotrophic host, etc.

The vector may or may not be capable of stable maintenance in the host. Where the vector is capable of stable maintenance, the cells will be screened for homologous integration of the vector into the genome of the host, where various techniques for curing the cells may be employed. Where the vector is not capable of stable maintenance, for example, where a temperature sensitive replication system is employed, one may change the temperature from the permissive temperature to the non-permissive temperature, so that the cells may be cured of the vector. In this case, only those cells having integration of the construct comprising the amplifiable gene and, when present, the selectable marker, will be able to survive selection.

Where a selectable marker is present, one may select for the presence of the targeting construct by means of the selectable marker. Where the selectable marker is not present, one may select for the presence of the construct by the amplifiable gene. For the neo gene or the herpes tk gene, one could employ a medium for growth of the transformants of about 0.1-1 mg/ml of G418 or may use HAT medium, respectively. Where DHFR is the amplifiable gene, the selective medium may include from about 0.01-0.5 μM of methotrexate or be deficient in glycine-hypoxanthine-thymidine and have dialysed serum (GHT media).

The DNA can be introduced into the expression host by a variety of techniques that include calcium phosphate/DNA co-precipitates, microinjection of DNA into the nucleus, electroporation, yeast protoplast fusion with intact cells, transfection, polycations, e.g., polybrene, polyornithine, etc., or the like. The DNA may be single or double stranded DNA, linear or circular. The various techniques for transforming mammalian cells are well known (see Keown et al., Methods Enzymol. (1989); Keown et al., Methods Enzymol. 185:527-537 (1990); Mansour et al., Nature 336:348-352, (1988)).

(d) Insect Constructs and Transformed Insect Cells

The present invention also relates to an insect recombinant vectors comprising exogenous genetic material. The present invention also relates to an insect cell comprising an insect recombinant vector. The present invention also relates to methods for obtaining a recombinant insect host cell, comprising introducing into an insect cell exogenous genetic material. In a preferred embodiment the exogenous genetic material includes a nucleic acid molecule of the present invention having a sequence selected from the group consisting of SEQ ID NO: 1 through SEQ ID NO: 294,310 or complements thereof or fragments of either.

The insect recombinant vector may be any vector which can be conveniently subjected to recombinant DNA procedures and can bring about the expression of the nucleic acid sequence. The choice of a vector will typically depend on the compatibility of the vector with the insect host cell into which the vector is to be introduced. The vector may be a linear or a closed circular plasmid. The vector system may be a single vector or plasmid or two or more vectors or plasmids which together contain the total DNA to be introduced into the genome of the insect host. In addition, the insect vector may be an expression vector. Nucleic acid molecules can be suitably inserted into a replication vector for expression in the insect cell under a suitable promoter for insect cells. Many vectors are available for this purpose and selection of the appropriate vector will depend mainly on the size of the nucleic acid molecule to be inserted into the vector and the particular host cell to be transformed with the vector. Each vector contains various components depending on its function (amplification of DNA or expression of DNA) and the particular host cell with which it is compatible. The vector components for insect cell transformation generally include, but are not limited to, one or more of the following: a signal sequence, origin of replication, one or more marker genes and an inducible promoter.

The insect vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the insect cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the insect host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the insect host cell and, furthermore, may be non-encoding or encoding sequences.

Baculovirus expression vectors (BEVs) have become important tools for the expression of foreign genes, both for basic research and for the production of proteins with direct clinical applications in human and veterinary medicine (Doerfler, Curr. Top. Microbiol. Immunol. 131:51-68 (1968); Luckow and Summers, Bio/Technology 6:47-55 (1988a); Miller, Annual Review of Microbiol. 42:177-199 (1988); Summers, Curr. Comm. Molecular Biology, Cold Spring Harbor Press, Cold Spring Harbor, N.Y. (1988)). BEVs are recombinant insect viruses in which the coding sequence for a chosen foreign gene has been inserted behind a baculovirus promoter in place of the viral gene, e.g., polyhedrin (Smith and Summers, U.S. Pat. No. 4,745,051).

The use of baculovirus vectors relies upon the host cells being derived from Lepidopteran insects such as Spodoptera frugiperda or Trichoplusia ni. The preferred Spodoptera frugiperda cell line is the cell line Sf9. The Spodoptera frugiperda Sf9 cell line was obtained from American Type Culture Collection (Manassas, Va.) and is assigned accession number ATCC CRL 1711 (Summers and Smith, A Manual of Methods for Baculovirus Vectors and Insect Cell Culture Procedures, Texas Ag. Exper. Station Bulletin No. 1555 (1988)). Other insect cell systems, such as the silkworm B. mori may also be used.

The proteins expressed by the BEVs are, therefore, synthesized, modified and transported in host cells derived from Lepidopteran insects. Most of the genes that have been inserted and produced in the baculovirus expression vector system have been derived from vertebrate species. Other baculovirus genes in addition to the polyhedrin promoter may be employed to advantage in a baculovirus expression system. These include immediate-early (alpha), delayed-early (β), late (γ), or very late (delta), according to the phase of the viral infection during which they are expressed. The expression of these genes occurs sequentially, probably as the result of a “cascade” mechanism of transcriptional regulation. (Guarino and Summers, J. Virol. 57:563-571 (1986); Guarino and Summers, J. Virol. 61:2091-2099 (1987); Guarino and Summers, Virol. 162:444-451 (1988)).

Insect recombinant vectors are useful as intermediates for the infection or transformation of insect cell systems. For example, an insect recombinant vector containing a nucleic acid molecule encoding a baculovirus transcriptional promoter followed downstream by an insect signal DNA sequence is capable of directing the secretion of the desired biologically active protein from the insect cell. The vector may utilize a baculovirus transcriptional promoter region derived from any of the over 500 baculoviruses generally infecting insects, such as for example the Orders Lepidoptera, Diptera, Orthoptera, Coleoptera and Hymenoptera, including for example but not limited to the viral DNAs of Autographa californica MNPV, Bombyx mori NPV, Trichoplusia ni MNPV, Rachiplusia ou MNPV or Galleria mellonella MNPV, wherein said baculovirus transcriptional promoter is a baculovirus immediate-early gene IEl or EN promoter; an immediate-early gene in combination with a baculovirus delayed-early gene promoter region selected from the group consisting of 39K and a HindIII-k fragment delayed-early gene; or a baculovirus late gene promoter. The immediate-early or delayed-early promoters can be enhanced with transcriptional enhancer elements. The insect signal DNA sequence may code for a signal peptide of a Lepidopteran adipokinetic hormone precursor or a signal peptide of the Manduca sexta adipokinetic hormone precursor (Summers, U.S. Pat. No. 5,155,037). Other insect signal DNA sequences include a signal peptide of the Orthoptera Schistocerca gregaria locust adipokinetic hormone precursor and the Drosophila melanogaster cuticle genes CP1, CP2, CP3 or CP4 or for an insect signal peptide having substantially a similar chemical composition and function (Summers, U.S. Pat. No. 5,155,037).

Insect cells are distinctly different from animal cells. Insects have a unique life cycle and have distinct cellular properties such as the lack of intracellular plasminogen activators in which are present in vertebrate cells. Another difference is the high expression levels of protein products ranging from 1 to greater than 500 mg/liter and the ease at which cDNA can be cloned into cells (Frasier, In Vitro Cell. Dev. Biol. 25:225 (1989); Summers and Smith, In: A Manual of Methods for Baculovirus Vectors and Insect Cell Culture Procedures, Texas Ag. Exper. Station Bulletin No. 1555 (1988)).

Recombinant protein expression in insect cells is achieved by viral infection or stable transformation. For viral infection, the desired gene is cloned into baculovirus at the site of the wild-type polyhedron gene (Webb and Summers, Technique 2:173 (1990); Bishop and Posse, Adv. Gene Technol. 1:55 (1990)). The polyhedron gene is a component of a protein coat in occlusions which encapsulate virus particles. Deletion or insertion in the polyhedron gene results the failure to form occlusion bodies. Occlusion negative viruses are morphologically different from occlusion positive viruses and enable one skilled in the art to identify and purify recombinant viruses.

The vectors of present invention preferably contain one or more selectable markers which permit easy selection of transformed cells. A selectable marker is a gene the product of which provides, for example biocide or viral resistance, resistance to heavy metals, prototrophy to auxotrophs and the like. Selection may be accomplished by co-transformation, e.g., as described in WO 91/17243, a nucleic acid sequence of the present invention may be operably linked to a suitable promoter sequence. The promoter sequence is a nucleic acid sequence which is recognized by the insect host cell for expression of the nucleic acid sequence. The promoter sequence contains transcription and translation control sequences which mediate the expression of the protein or fragment thereof. The promoter may be any nucleic acid sequence which shows transcriptional activity in the insect host cell of choice and may be obtained from genes encoding polypeptides either homologous or heterologous to the host cell.

For example, a nucleic acid molecule encoding a protein or fragment thereof may also be operably linked to a suitable leader sequence. A leader sequence is a nontranslated region of a mRNA which is important for translation by the fungal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the insect host cell of choice may be used in the present invention.

A polyadenylation sequence may also be operably linked to the 3′ terminus of the nucleic acid sequence of the present invention. The polyadenylation sequence is a sequence which when transcribed is recognized by the insect host to add polyadenosine residues to transcribed mRNA. The polyadenylation sequence may be native to the nucleic acid sequence encoding the protein or fragment thereof or may be obtained from foreign sources. Any polyadenylation sequence which is functional in the fungal host of choice may be used in the present invention.

To avoid the necessity of disrupting the cell to obtain the protein or fragment thereof and to minimize the amount of possible degradation of the expressed polypeptide within the cell, it is preferred that expression of the polypeptide gene gives rise to a product secreted outside the cell. To this end, the protein or fragment thereof of the present invention may be linked to a signal peptide linked to the amino terminus of the protein or fragment thereof. A signal peptide is an amino acid sequence which permits the secretion of the protein or fragment thereof from the insect host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in translation reading frame with the segment of the coding region which encodes the secreted protein or fragment thereof.

At present, a mode of achieving secretion of a foreign gene product in insect cells is by way of the foreign gene's native signal peptide. Because the foreign genes are usually from non-insect organisms, their signal sequences may be poorly recognized by insect cells and hence, levels of expression may be suboptimal. However, the efficiency of expression of foreign gene products seems to depend primarily on the characteristics of the foreign protein. On average, nuclear localized or non-structural proteins are most highly expressed, secreted proteins are intermediate and integral membrane proteins are the least expressed. One factor generally affecting the efficiency of the production of foreign gene products in a heterologous host system is the presence of native signal sequences (also termed presequences, targeting signals, or leader sequences) associated with the foreign gene. The signal sequence is generally coded by a DNA sequence immediately following (5′ to 3′) the translation start site of the desired foreign gene.

The expression dependence on the type of signal sequence associated with a gene product can be represented by the following example: If a foreign gene is inserted at a site downstream from the translational start site of the baculovirus polyhedrin gene so as to produce a fusion protein (containing the N-terminus of the polyhedrin structural gene), the fused gene is highly expressed. But less expression is achieved when a foreign gene is inserted in a baculovirus expression vector immediately following the transcriptional start site and totally replacing the polyhedrin structural gene.

Insertions into the region −50 to −1 significantly alter (reduce) steady state transcription which, in turn, reduces translation of the foreign gene product. Use of the pVL941 vector optimizes transcription of foreign genes to the level of the polyhedrin gene transcription. Even though the transcription of a foreign gene may be optimal, optimal translation may vary because of several factors involving processing: signal peptide recognition, mRNA and ribosome binding, glycosylation, disulfide bond formation, sugar processing, oligomerization, for example.

The properties of the insect signal peptide are expected to be more optimal for the efficiency of the translation process in insect cells than those from vertebrate proteins. This phenomenon can generally be explained by the fact that proteins secreted from cells are synthesized as precursor molecules containing hydrophobic N-terminal signal peptides. The signal peptides direct transport of the select protein to its target membrane and are then cleaved by a peptidase on the membrane, such as the endoplasmic reticulum, when the protein passes through it.

Another exemplary insect signal sequence is the sequence encoding for Drosophila cuticle proteins such as CP1, CP2, CP3 or CP4 (Summers, U.S. Pat. No. 5,278,050). Most of a 9 kb region of the Drosophila genome containing genes for the cuticle proteins has been sequenced. Four of the five cuticle genes contains a signal peptide coding sequence interrupted by a short intervening sequence (about 60 base pairs) at a conserved site. Conserved sequences occur in the 5′ mRNA untranslated region, in the adjacent 35 base pairs of upstream flanking sequence and at −200 base pairs from the mRNA start position in each of the cuticle genes.

Standard methods of insect cell culture, cotransfection and preparation of plasmids are set forth in Summers and Smith (Summers and Smith, A Manual of Methods for Baculovirus Vectors and Insect Cell Culture Procedures, Texas Agricultural Experiment Station Bulletin No. 1555, Texas A&M University (1987)). Procedures for the cultivation of viruses and cells are described in Volkman and Summers, J. Virol 19:820-832 (1975) and Volkman et al., J. Virol 19:820-832 (1976).

(e) Bacterial Constructs and Transformed Bacterial Cells

The present invention also relates to a bacterial recombinant vector comprising exogenous genetic material. The present invention also relates to a bacteria cell comprising a bacterial recombinant vector. The present invention also relates to methods for obtaining a recombinant bacteria host cell, comprising introducing into a bacterial host cell exogenous genetic material. In a preferred embodiment the exogenous genetic material includes a nucleic acid molecule of the present invention having a sequence selected from the group consisting of SEQ ID NO: 1 through SEQ ID NO: 294,310 or complements thereof or fragments of either.

The bacterial recombinant vector may be any vector which can be conveniently subjected to recombinant DNA procedures. The choice of a vector will typically depend on the compatibility of the vector with the bacterial host cell into which the vector is to be introduced. The vector may be a linear or a closed circular plasmid. The vector system may be a single vector or plasmid or two or more vectors or plasmids which together contain the total DNA to be introduced into the genome of the bacterial host. In addition, the bacterial vector may be an expression vector. Nucleic acid molecules encoding protein homologues or fragments thereof can, for example, be suitably inserted into a replicable vector for expression in the bacterium under the control of a suitable promoter for bacteria. Many vectors are available for this purpose and selection of the appropriate vector will depend mainly on the size of the nucleic acid to be inserted into the vector and the particular host cell to be transformed with the vector. Each vector contains various components depending on its function (amplification of DNA or expression of DNA) and the particular host cell with which it is compatible. The vector components for bacterial transformation generally include, but are not limited to, one or more of the following: a signal sequence, an origin of replication, one or more marker genes and an inducible promoter.

In general, plasmid vectors containing replicon and control sequences that are derived from species compatible with the host cell are used in connection with bacterial hosts. The vector ordinarily carries a replication site, as well as marking sequences that are capable of providing phenotypic selection in transformed cells. For example, E. coli is typically transformed using pBR322, a plasmid derived from an E. coli species (see, e.g., Bolivar et al., Gene 2:95 (1977)). pBR322 contains genes for ampicillin and tetracycline resistance and thus provides easy means for identifying transformed cells. The pBR322 plasmid, or other microbial plasmid or phage, also generally contains, or is modified to contain, promoters that can be used by the microbial organism for expression of the selectable marker genes.

Nucleic acid molecules encoding protein or fragments thereof may be expressed not only directly, but also as a fusion with another polypeptide, preferably a signal sequence or other polypeptide having a specific cleavage site at the N-terminus of the mature polypeptide. In general, the signal sequence may be a component of the vector, or it may be a part of the polypeptide DNA that is inserted into the vector. The heterologous signal sequence selected should be one that is recognized and processed (i.e., cleaved by a signal peptidase) by the host cell. For bacterial host cells that do not recognize and process the native polypeptide signal sequence, the signal sequence is substituted by a bacterial signal sequence selected, for example, from the group consisting of the alkaline phosphatase, penicillinase, lpp, or heat-stable enterotoxin II leaders.

Both expression and cloning vectors contain a nucleic acid sequence that enables the vector to replicate in one or more selected host cells. Generally, in cloning vectors this sequence is one that enables the vector to replicate independently of the host chromosomal DNA and includes origins of replication or autonomously replicating sequences. Such sequences are well known for a variety of bacteria. The origin of replication from the plasmid pBR322 is suitable for most Gram-negative bacteria.

Expression and cloning vectors also generally contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous protein homologue or fragment thereof produce a protein conferring drug resistance and thus survive the selection regimen.

The expression vector for producing a protein or fragment thereof can also contains an inducible promoter that is recognized by the host bacterial organism and is operably linked to the nucleic acid encoding, for example, the nucleic acid molecule encoding the protein homologue or fragment thereof of interest. Inducible promoters suitable for use with bacterial hosts include the β-lactamase and lactose promoter systems (Chang et al., Nature 275:615 (1978); Goeddel et al., Nature 281:544 (1979)), the arabinose promoter system (Guzman et al., J. Bacteriol. 174:7716-7728 (1992)), alkaline phosphatase, a tryptophan (trp) promoter system (Goeddel, Nucleic Acids Res. 8:4057 (1980); EP 36,776) and hybrid promoters such as the tac promoter (deBoer et al., Proc. Natl. Acad. Sci. (USA) 80:21-25 (1983)). However, other known bacterial inducible promoters are suitable (Siebenlist et al., Cell 20:269 (1980)).

Promoters for use in bacterial systems also generally contain a Shine-Dalgarno (S.D.) sequence operably linked to the DNA encoding the polypeptide of interest. The promoter can be removed from the bacterial source DNA by restriction enzyme digestion and inserted into the vector containing the desired DNA.

Construction of suitable vectors containing one or more of the above-listed components employs standard ligation techniques. Isolated plasmids or DNA fragments are cleaved, tailored and re-ligated in the form desired to generate the plasmids required. Examples of available bacterial expression vectors include, but are not limited to, the multifunctional E. coli cloning and expression vectors such as Bluescript™ (Stratagene, La Jolla, Calif.), in which, for example, encoding an A. nidulans protein homologue or fragment thereof homologue, may be ligated into the vector in frame with sequences for the amino-terminal Met and the subsequent 7 residues of β-galactosidase so that a hybrid protein is produced; pIN vectors (Van Heeke and Schuster, J. Biol. Chem. 264:5503-5509 (1989)); and the like. pGEX vectors (Promega, Madison Wis. U.S.A.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the GST moiety at will.

Suitable host bacteria for a bacterial vector include archaebacteria and eubacteria, especially eubacteria and most preferably Enterobacteriaceae. Examples of useful bacteria include Escherichia, Enterobacter, Azotobacter, Erwinia, Bacillus, Pseudomonas, Klebsiella, Proteus, Salmonella, Serratia, Shigella, Rhizobia, Vitreoscilla and Paracoccus. Suitable E. coli hosts include E. coli W3110 (American Type Culture Collection (ATCC) 27,325, Manassas, Va. U.S.A.), E. coli 294 (ATCC 31,446), E. coli B and E. coli X1776 (ATCC 31,537). These examples are illustrative rather than limiting. Mutant cells of any of the above-mentioned bacteria may also be employed. It is, of course, necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. E. coli strain W3110 is a preferred host or parent host because it is a common host strain for recombinant DNA product fermentations. Preferably, the host cell should secrete minimal amounts of proteolytic enzymes.

Host cells are transfected and preferably transformed with the above-described vectors and cultured in conventional nutrient media modified as appropriate for inducing promoters, selecting transformants, or amplifying the genes encoding the desired sequences.

Numerous methods of transfection are known to the ordinarily skilled artisan, for example, calcium phosphate and electroporation. Depending on the host cell used, transformation is done using standard techniques appropriate to such cells. The calcium treatment employing calcium chloride, as described in section 1.82 of Sambrook et al., Molecular Cloning: A Laboratory Manual, New York: Cold Spring Harbor Laboratory Press, (1989), is generally used for bacterial cells that contain substantial cell-wall barriers. Another method for transformation employs polyethylene glycol/DMSO, as described in Chung and Miller (Chung and Miller, Nucleic Acids Res. 16:3580 (1988)). Yet another method is the use of the technique termed electroporation.

Bacterial cells used to produce the polypeptide of interest for purposes of this invention are cultured in suitable media in which the promoters for the nucleic acid encoding the heterologous polypeptide can be artificially induced as described generally, e.g., in Sambrook et al., Molecular Cloning: A Laboratory Manual, New York: Cold Spring Harbor Laboratory Press, (1989). Examples of suitable media are given in U.S. Pat. Nos. 5,304,472 and 5,342,763.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning: A Laboratory Manual, Cold Spring Harbor Press (1989); Mailga et al., Methods in Plant Molecular Biology, Cold Spring Harbor Press (1995); Birren et al., Genome Analysis Analyzing DNA, 1, Cold Spring Harbor, N.Y. (1997).

(f) Algal Construets and Algal Transformants

The present invention also relates to an algal recombinant vector comprising exogenous genetic material. The present invention also relates to an algal cell comprising an algal recombinant vector. The present invention also relates to methods for obtaining a recombinant algal host cell comprising introducing into an algal host cell exogenous genetic material.

Exogenous genetic material is any genetic material, whether naturally occurring or otherwise, from any source that is capable of being inserted into any organism. Exogenous genetic material may be transferred into an algal cell. In a preferred embodiment the exogenous genetic material includes a nucleic acid molecule having a sequence selected from the group consisting of SEQ ID NO: 1 through SEQ ID NO: XXXX or complements thereof.

The algal recombinant vector may be any vector which can be conveniently subjected to recombinant DNA procedures. The choice of a vector will typically depend on the compatibility of the vector with the algal host cell into which the vector is to be introduced. The vector may be a linear or a closed circular plasmid. The vector system may be a single vector or plasmid or two or more vectors or plasmids which together contain the total DNA to be introduced into the genome of the algal host.

The algal vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the algal cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the algal host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the algal host cell, and, furthermore, may be non-encoding or encoding sequences.

The vectors of the present invention preferably contain one or more selectable markers which permit easy selection of transformed cells. A selectable marker is a gene, the product of which confers upon an algal cell resistance to a compound to which the algal would otherwise be sensitive. The compound can be selected from the group consisting of antibiotics, fungicides, herbicides, and heavy metals. The selectable marker may be selected from any known or subsequently identified selectable markers, including markers derived from algal, fungal, and baterial sources. Preferred selectable markers can be selected from the group including, but not limited to, amdS (acetamidase), argB (ornithine carbamoyltransferase), bar (phosphinothricin acetyltransferase), ble (bleomycin binding protein), cat (chloramphenicol acetyltransferase), hygB (hygromycin B phosphotransferase), nat (nourseothricin acetyltransferase), niaD (nitrate reductase), neo (neomycin phosphotransferase), pac (puromycin acetyltransferase), pyrG (orotidine-5′-phosphate decarboxylase), sat (streptothricin acetyltransferase), sC (sulfate adenyltransferase), trpC (anthranilate synthase), and glyphosate resistant EPSPS genes. Furthermore, selection may be accomplished by co-transformation, e.g., as described in WO 91/17243, herein incorporated by reference in its entirety.

A nucleic acid sequence of the present invention may be operably linked to a suitable promoter sequence. The promoter sequence is a nucleic acid sequence which is recognized by the algal host cell for expression of the nucleic acid sequence. The promoter sequence contains transcription and translation control sequences which mediate the expression of the protein or fragment thereof.

A promoter may be any nucleic acid sequence which shows transcriptional activity in the algal host cell of choice and may be obtained from genes encoding polypeptides either homologous or heterologous to the host cell. Examples of suitable promoters for directing the transcription of a nucleic acid construct of the invention in an algal host are light harvesting protein promoters obtained from photosynthetic organisms, Chlorella virus methyltransferase promoters, CaMV 35 S promoter, PL promoter from bacteriophage λ, nopaline synthase promoter from the Ti plasmid of Agrobacterium tumefaciens, and bacterial trp promotor.

A protein or fragment thereof encoding nucleic acid molecule of the present invention may also be operably linked to a terminator sequence at its 3′ terminus. The terminator sequence may be native to the nucleic acid sequence encoding the protein or fragment thereof or may be obtained from foreign sources. Any terminator which is functional in the algal host cell of choice may be used in the present invention.

A protein or fragment thereof encoding nucleic acid molecule of the present invention may also be operably linked to a suitable leader sequence. A leader sequence is a nontranslated region of a mRNA which is important for translation by the algal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the algal host cell of choice may be used in the present invention.

A polyadenylation sequence may also be operably linked to the 3′ terminus of the nucleic acid sequence of the present invention. The polyadenylation sequence is a sequence which when transcribed is recognized by the algal host to add polyadenosine residues to transcribed mRNA. The polyadenylation sequence may be native to the nucleic acid sequence encoding the protein or fragment thereof or may be obtained from foreign sources. Any polyadenylation sequence which is functional in the algal host of choice may be used in the present invention.

The procedures used to ligate the elements described above to construct the recombinant expression vector of the present invention are well known to one skilled in the art (see, for example, Sambrook, 2nd ed., et al., Molecular Cloning, A Laboratory Manual Cold Spring Harbor, N.Y., (1989), herein incorporated by reference in its entirety).

The present invention also relates to recombinant algal host cells produced by the methods of the present invention which are advantageously used with the recombinant vector of the present invention. The cell is preferably transformed with a vector comprising a nucleic acid sequence of the invention followed by integration of the vector into the host chromosome. The choice of algal host cells will to a large extent depend upon the gene encoding the protein or fragment thereof and its source.

Algal cells may be transformed by a variety of known techniques, including but not limit to, microprojectile bombardment, protoplast fusion, electroporation, microinjection, and vigorous agitation in the presence of glass beads. Suitable procedures for transformation of green algal host cells are described in EP 108 580, herein incorporated by reference in its entirety. A suitable method of transforming Chlorella species is described by Jarvis and Brown, Curr. Genet. 19: 317-321 (1991), herein incorporated by reference in its entirety. A suitable method of transforming cells of diatom Phaeodactylum tricornutum species is described in WO 97/39106, herein incorporated by reference in its entirety. Chlorophyll C-containing algae may be transformed using the procedures described in U.S. Pat. No. 5,661,017, herein incorporated by reference in its entirety.

The expressed protein or fragment thereof may be detected using methods known in the art that are specific for the particular protein or fragment. These detection methods may include the use of specific antibodies, formation of an enzyme product, or disappearance of an enzyme substrate. For example, if the protein or fragment thereof has enzymatic activity, an enzyme assay may be used. Alternatively, if polyclonal or monoclonal antibodies specific to the protein or fragment thereof are available, immunoassays may be employed using the antibodies to the protein or fragment thereof. The techniques of enzyme assay and immunoassay are well known to those skilled in the art.

The resulting protein or fragment thereof may be recovered by methods known in the arts. For example, the protein or fragment thereof may be recovered from the nutrient medium by conventional procedures including, but not limited to, centrifugation, filtration, extraction, spray-drying, evaporation, or precipitation. The recovered protein or fragment thereof may then be further purified by a variety of chromatographic procedures, e.g., ion exchange chromatography, gel filtration chromatography, affinity chromatography, or the like.

(g) Computer Readable Media

The nucleotide sequence provided in SEQ ID NO: 1 through SEQ ID NO: 294,310 or fragment thereof, or complement thereof, or a nucleotide sequence at least 90% identical, preferably 95%, identical even more preferably 99% or 100% identical to the sequence provided in SEQ ID NO: 1 through SEQ ID NO: 294,310 or fragment thereof, or complement thereof, can be “provided” in a variety of mediums to facilitate use. Such a medium can also provide a subset thereof in a form that allows a skilled artisan to examine the sequences.

In a preferred embodiment of the present invention computer readable media may be prepared that comprise nucleic acid sequences where preferably at least 10%, preferably at least 25%, more preferably at least 50% and even more preferably at least 75%, 80%, 85%, 90% or 95% of the nucleic acid sequences are selected from the group of nucleic acid molecules that specifically hybridize to one or more nucleic acid molecule having a nucleic acid sequence selected from the group of SEQ ID NO: 1 through SEQ ID NO: 294,310 or complement thereof or fragments of either.

In another preferred embodiment of the present invention computer readable media may be prepared that comprise nucleic acid sequences where preferably at least 10%, preferably at least 25%, more preferably at least 50% and even more preferably at least 75%, 80%, 85%, 90% or 95% of the nucleic acid sequences are selected from the group of nucleic acid molecules having a nucleic acid sequence selected from the group of SEQ ID NO: 1 through SEQ ID NO: 294,310 or complements thereof.

In a more preferred embodiment of the present invention, the computer readable media comprises a nucleic acid sequence and/or collection of nucleic acid sequences of the present invention associated with a biochemical process or activity where the process or activity is preferably selected from photosynthetic activity, carbohydrate metabolism, amino acid synthesis or degradation, plant hormone or other regulatory molecules, phenolic metabolism, and lipid metabolism, and more preferably selected from the group consisting of biosynthesis of tetrapyrroles, phytochrome metabolism, carbon assimilation, glycolysis and gluconeogenesis metabolism, sucrose metabolism, starch metabolism, phosphogluconate metabolism, galactomannan metabolism, raffinose metabolism, complex carbohydrate synthesis/degradation, phytic acid metabolism, methionine biosynthesis, methionine degradation, lysine metabolism, arginine metabolism, proline metabolism, glutamate/glutamine metabolism, aspartate/asparagine metabolism, cytokinin metabolism, gibberellin metabolism, ethylene metabolism, jasmonic acid metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, and fatty acid metabolism, and even more preferably selected from the group consisting of: glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

In an even more preferred embodiment of the present invention, the computer readable media comprises a nucleic acid sequence and/or collection of nucleic acid sequences of the present invention where the nucleic acid sequence and/or collection of nucleic acid sequences are associated with a component or attribute of at least two, more preferable at least three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five or forty six biochemical processes or activities where the biochemical processes or activities are selected from the following: photosynthetic activity, carbohydrate metabolism, amino acid synthesis or degradation, plant hormone or other regulatory molecules, phenolic metabolism, lipid metabolism, biosynthesis of tetrapyrroles, phytochrome metabolism, carbon assimilation, glycolysis and gluconeogenesis metabolism, sucrose metabolism, starch metabolism, phosphogluconate metabolism, galactomannan metabolism, raffinose metabolism, complex carbohydrate synthesis/degradation, phytic acid metabolism, methionine biosynthesis, methionine degradation, lysine metabolism, arginine metabolism, proline metabolism, glutamate/glutamine, aspartate/asparagine metabolism, cytokinin metabolism, gibberellin metabolism, ethylene metabolism, jasmonic acid synthesis metabolism, transcription factors, R-genes, plant proteases, protein kinases, antifungal proteins, nitrogen transporters, sugar transporters, shikimate metabolism, isoflavone metabolism, phenylpropanoid metabolism, isoprenoid metabolism, β-oxidation lipid metabolism, fatty acid metabolism, glycolysis metabolism, gluconeogenesis metabolism, sucrose metabolism, sucrose catabolism, reductive pentose phosphate cycle, regulation of C3 photosynthesis, C4 pathway carbon assimilation, enzymes involved in the C4 pathway, carotenoid metabolism, tocopherol metabolism, phytosterol metabolism, brassinoid metabolism, and proline metabolism.

In one application of this embodiment, a nucleotide sequence of the present invention can be recorded on computer readable media. As used herein, “computer readable media” refers to any medium that can be read and accessed directly by a computer. Such media include, but are not limited to: magnetic storage media, such as floppy discs, hard disc, storage medium and magnetic tape: optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the presently known methods for recording information on computer readable medium to generate media comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g., text file or database) in order to obtain computer readable medium having recorded thereon the nucleotide sequence information of the present invention.

By providing one or more of nucleotide sequences of the present invention, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215:403-410 (1990)) and BLAZE (Brutlag et al., Comp. Chem. 17:203-207 (1993)) search algorithms on a Sybase system can be used to identify open reading frames (ORFs) within the genome that contain homology to ORFs or proteins from other organisms. Such ORFs are protein-encoding fragments within the sequences of the present invention and are useful in producing commercially important proteins such as enzymes used in amino acid biosynthesis, metabolism, transcription, translation, RNA processing, nucleic acid and a protein degradation, protein modification and DNA replication, restriction, modification, recombination and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware means, software means and data storage means used to analyze the nucleotide sequence information of the present invention. The minimum hardware means of the computer-based systems of the present invention comprises a central processing unit (CPU), input means, output means and data storage means. A skilled artisan can readily appreciate that any one of the currently available computer-based system are suitable for use in the present invention.

As indicated above, the computer-based systems of the present invention comprise a data storage means having stored therein a nucleotide sequence of the present invention and the necessary hardware means and software means for supporting and implementing a search means. As used herein, “data storage means” refers to memory that can store nucleotide sequence information of the present invention, or a memory access means which can access manufactures having recorded thereon the nucleotide sequence information of the present invention. As used herein, “search means” refers to one or more programs which are implemented on the computer-based system to compare a target sequence or target structural motif with the sequence information stored within the data storage means. Search means are used to identify fragments or regions of the sequence of the present invention that match a particular target sequence or target motif. A variety of known algorithms are disclosed publicly and a variety of commercially available software for conducting search means are available can be used in the computer-based systems of the present invention. Examples of such software include, but are not limited to, MacPattern (EMBL), BLASTIN and BLASTIX (NCBIA). One of the available algorithms or implementing software packages for conducting homology searches can be adapted for use in the present computer-based systems.

The most preferred sequence length of a target sequence is from about 10 to 100 amino acids or from about 30 to 300 nucleotide residues. However, it is well recognized that during searches for commercially important fragments of the nucleic acid molecules of the present invention, such as sequence fragments involved in gene expression and protein processing, may be of shorter length.

As used herein, “a target structural motif,” or “target motif,” refers to any rationally selected sequence or combination of sequences in which the sequences the sequence(s) are chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

Thus, the present invention further provides an input means for receiving a target sequence, a data storage means for storing the target sequences of the present invention sequence identified using a search means as described above and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences which contain various amounts of the target sequence or target motif and identifies the degree of homology contained in the identified fragment.

A variety of comparing means can be used to compare a target sequence or target motif with the data storage means to identify sequence fragments sequence of the present invention. For example, implementing software which implement the BLAST and BLAZE algorithms (Altschul et al., J. Mol. Biol. 215:403-410 (1990)) can be used to identify open frames within the nucleic acid molecules of the present invention. A skilled artisan can readily recognize that any one of the publicly available homology search programs can be used as the search means for the computer-based systems of the present invention.

Having now generally described the invention, the same will be more readily understood through reference to the following examples which are provided by way of illustration and are not intended to be limiting of the present invention, unless specified.

### EXAMPLE 1

The SATMONN01 (MONN01) cDNA library is a normalized library generated from maize (DK604, Dekalb Genetics, Dekalb, Ill. U.S.A.) total leaf tissue at the V6 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when maize plants are at the 6-leaf development stage. The older, more juvenile leaves, which are in a basal position, as well as the younger, more adult leaves, which are more apical are cut at the base of the leaves. The leaves are then pooled and immediately transferred to liquid nitrogen containers in which the pooled leaves are crushed. The harvested tissue is then stored at −80° C. until RNA preparation. The RNA is purified from the stored tissue. The library is normalized in one round using conditions adapted from Soares et al., Proc. Natl. Acad. Sci. (U.S.A.) 91:9928 (1994), the entirety of which is herein incorporated by reference and Bonaldo et al., Genome Res. 6: 791 (1996), the entirety of which is herein incorporated by reference except that a longer (48-hours/round) reannealing hybridization was used. SATMON004 is a leaf tissue library from the same donor.

The SATMON001 cDNA library is generated from maize (B73, Illinois Foundation Seeds, Champaign, Ill. U.S.A.) immature tassels at the V6 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tassel tissue from maize plants is collected at the V6 stage. At that stage the tassel is an immature tassel of about 2-3 cm in length. Tassels are removed and frozen in liquid nitrogen. The harvested tissue is then stored at −80° C. until RNA preparation. The RNA is purified from the stored tissue and the cDNA library is constructed as described in Example 2.

The SATMON003 library is generated from maize (B73×Mo17, Illinois Foundation Seeds, Champaign, Ill. U.S.A.) roots at the V6 developmental stage. Seeds are planted at a depth of approximately 3 cm into 2-3 inch peat pots containing Metro 200 growing medium. After 2-3 weeks growth, the seedlings are transplanted into 10 inch pots containing the Metro 200 growing medium. Plants are watered daily before transplantation and approximately 3 times a week after transplantation. Peters 15-16-17 fertilizer is applied approximately three times per week after transplanting at a concentration of 150 ppm N. Two to three times during the life time of the plant from transplanting to flowering a total of approximately 900 mg Fe is added to each pot. Maize plants are grown in a greenhouse in approximately 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is at the V6 leaf development stage. The root system is cut from maize plant and washed with water to free it from the soil. The tissue is then immediately frozen in liquid nitrogen. The harvested tissue is then stored at −80° C. until RNA preparation. The RNA is purified from the stored tissue and the cDNA library is constructed as described in Example 2.

The SATMON004 cDNA library is generated from maize (B73×Mo17, Illinois Foundation Seeds, Champaign, Ill. U.S.A.) total leaf tissue at the V6 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is at the V6-leaf development stage. The older, more juvenile leaves, which are in a basal position, as well as the younger, more adult leaves, which are more apical are cut at the base of the leaves. The leaves are then pooled and immediately transferred to liquid nitrogen containers in which the pooled leaves are crushed. The harvested tissue is then stored at −80° C. until RNA preparation. The RNA is purified from the stored tissue and the cDNA library is constructed as described in Example 2.

The SATMON005 cDNA library is generated from maize (B73×Mo17, Illinois Foundation Seeds, Champaign Ill., U.S.A.) root tissue at the V6 development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is at the 6-leaf development stage. The root system is cut from the mature maize plant and washed with water to free it from the soil. The tissue is immediately frozen in liquid nitrogen and the harvested tissue is then stored at −80° C. until RNA preparation. The RNA is purified from the stored tissue and the cDNA library is constructed as described in Example 2.

The SATMON006 cDNA library is generated from maize (B73×Mo17, Illinois Foundation Seeds, Champaign Ill., U.S.A.) total leaf tissue at the V6 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is at the 6-leaf development stage. The older more juvenile leaves, which are in a basal position, as well as the younger more adult leaves, which are more apical are cut at the base of the leaves. The leaves are then pooled and immediately transferred to liquid nitrogen containers in which the pooled leaves are crushed. The harvested tissue is then stored at −80° C. until RNA preparation. The RNA is purified from the stored tissue and the cDNA library is constructed as described in Example 2.

The SATMON007 cDNA library is generated from the primary root tissue of 5 day old maize (DK604, Dekalb Genetics, Dekalb, Ill. U.S.A.) seedlings. Seeds are planted on a moist filter paper on a covered tray that is kept in the dark until germination (one day). After germination, the trays, along with the moist paper, are moved to a greenhouse where the maize plants are grown in the greenhouse in 15 hr day/9 hr night cycles for approximately 5 days. The daytime temperature is approximately 80° F. and the nighttime temperature is approximately 70° F. Supplemental lighting is provided by 1000 W sodium vapor lamps. The primary root tissue is collected when the seedlings are 5 days old. At this stage, the primary root (radicle) is pushed through the coleorhiza which itself is pushed through the seed coat. The primary root, which is about 2-3 cm long, is cut and immediately frozen in liquid nitrogen and then stored at −80° C. until RNA preparation. The RNA is purified from the stored tissue and the cDNA library is constructed as described in Example 2.

The SATMON008 cDNA library is generated from the primary shoot (coleoptile 2-3 cm) of maize (DK604, Dekalb Genetics, Dekalb, Ill. U.S.A.) seedlings which are approximately 5 days old. Seeds are planted on a moist filter paper on a covered tray that is kept in the dark until germination (one day). Then the trays containing the seeds are moved to a greenhouse at 15 hr daytime/9 hr nighttime cycles and grown until they are 5 days post germination. The daytime temperature is approximately 80° F. and the nighttime temperature is approximately 70° F. Tissue is collected when the seedlings are 5 days old. At this stage, the primary shoot (coleoptile) is pushed through the seed coat and is about 2-3 cm long. The coleoptile is dissected away from the rest of the seedling, immediately frozen in liquid nitrogen and then stored at −80° C. until RNA preparation. The RNA is purified from the stored tissue and the cDNA library is constructed as described in Example 2.

The SATMON009 cDNA library is generated from maize (DK604, Dekalb Genetics, Dekalb, Ill. U.S.A.) leaves at the 8 leaf stage (V8 plant development stage). Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is 80° F. and the nighttime temperature is 70° F. Supplemental lighting is provided by 1000 W sodium vapor lamps. Tissue is collected when the maize plant is at the 8-leaf development stage. The older more juvenile leaves, which are in a basal position, as well as the younger more adult leaves, which are more apical, are cut at the base of the leaves. The leaves are then pooled and then immediately transferred to liquid nitrogen containers in which the pooled leaves are crushed. The harvested tissue is then stored at −80° C. until RNA preparation. The RNA is purified from the stored tissue and the cDNA library is constructed as described in Example 2.

The SATMON010 cDNA library is generated from maize (DK604, Dekalb Genetics, Dekalb, Ill. U.S.A.) root tissue at the V8 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is 80° F. and the nighttime temperature is 70° F. Supplemental lighting is provided by 1000 W sodium vapor lamps. Tissue is collected when the maize plant is at the V8 development stage. The root system is cut from this mature maize plant and washed with water to free it from the soil. The tissue is immediately frozen in liquid nitrogen. The harvested tissue is then stored at −80° C. until RNA preparation. The RNA is purified from the stored tissue and the cDNA library is constructed as described in Example 2.

The SATMON011 cDNA library is generated from undeveloped maize (DK604, Dekalb Genetics, Dekalb, Ill. U.S.A.) leaf at the V6 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is at the 6-leaf development stage. The second youngest leaf which is at the base of the apical leaf of V6 stage maize plant is cut at the base and immediately transferred to liquid nitrogen containers in which the leaf is crushed. The harvested tissue is then stored at −80° C. until RNA preparation. The RNA is purified from the stored tissue and the cDNA library is constructed as described in Example 2.

The SATMON012 cDNA library is generated from 2 day post germination maize (DK604, Dekalb Genetics, Dekalb, Ill. U.S.A.) seedlings. Seeds are planted on a moist filter paper on a covered tray that is kept in the dark until germination (one day). Then the trays containing the seeds are moved to the greenhouse and grown at 15 hr daytime/9 hr nighttime cycles until 2 days post germination. The daytime temperature is approximately 80° F. and the nighttime temperature is approximately 70° F. Tissue is collected when the seedlings are 2 days old. At the two day stage, the coleorhiza is pushed through the seed coat and the primary root (the radicle) is pierced the coleorhiza but is barely visible. Also, at this two day stage, the coleoptile is just emerging from the seed coat. The 2 days post germination seedlings are then immersed in liquid nitrogen and crushed. The harvested tissue is stored at −80° C. until preparation of total RNA. The RNA is purified from the stored tissue and the cDNA library is constructed as described in Example 2.

The SATMON013 cDNA library is generated from apical maize (DK604, Dekalb Genetics, Dekalb, Ill. U.S.A.) meristem founder at the V4 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Prior to tissue collection, the plant is at the V4 leaf stage. The lead at the apex of the V4 stage maize plant is referred to as the meristem founder. This apical meristem founder is cut, immediately frozen in liquid nitrogen and crushed. The harvested tissue is then stored at −80° C. until RNA preparation. The RNA is purified from the stored tissue and the cDNA library is constructed as described in Example 2.

The SATMON014 cDNA library is generated from maize (DK604, Dekalb Genetics, Dekalb, Ill. U.S.A.) endosperm at fourteen days after pollination. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. After the V10 stage, ear shoots are ready for fertilization. At this stage, the ear shoots are enclosed in a paper bag before silk emergence to withhold the pollen. The ear shoots are pollinated and 14 days after pollination, the ears are pulled out and then the kernels are plucked out of the ears. Each kernel is then dissected into the embryo and the endosperm and the aleurone layer is removed. After dissection, the endosperms are immediately frozen in liquid nitrogen and then stored at −80° C. until RNA preparation. The RNA is purified from the stored tissue and the cDNA library is constructed as described in Example 2.

The SATMON016 cDNA library is generated from maize (DK604, Dekalb Genetics, Dekalb, Ill. U.S.A.) sheath tissue collected at the V8 developmental stage. Seeds are planted in a depth of approximately 3 cm in solid into 2-3 inch pots containing Metro growing medium. After 2-3 weeks growth, they are transplanted into 10 inch pots containing the same. Plants are watered daily before transplantation and approximately the times a week after transplantation. Peters 15-16-17 fertilizer is applied approximately three times per week after transplanting, at a strength of 150 ppm N. Two to three times during the life time of the plant from transplanting to flowering, a total of approximately 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. When the maize plants are at the V8 stage, the 5^{th} and 6^{th} leaves from the bottom exhibit fully developed leaf blades. At the base of these leaves, the ligule is differentiated and the leaf blade is joined to the sheath. The sheath is dissected away from the base of the leaf then the sheath is frozen in liquid nitrogen and crushed. The tissue is then stored at −80° C. until RNA preparation. The RNA is purified from the stored tissue and the cDNA library is constructed as described in Example 2.

The SATMON017 cDNA library is generated from maize (DK604, Dekalb Genetics, Dekalb, Ill. U.S.A.) embryo collected from plants at twenty one days after pollination. Seeds are planted at a depth of approximately 3 cm into 2-3 inch peat pots containing Metro 200 growing medium. After 2-3 weeks growth the seeds are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. After the V10 stage, the ear shoots of maize plant, which are ready for fertilization, are enclosed in a paper bag before silk emergence to withhold the pollen. The ear shoots are fertilized and 21 days after pollination, the ears are pulled out and the kernels are plucked out of the ears. Each kernel is then dissected into the embryo and the endosperm and the aleurone layer is removed. After dissection, the embryos are immediately frozen in liquid nitrogen and then stored at −80° C. until RNA preparation. The RNA is purified from the stored tissue and the cDNA library is constructed as described in Example 2.

The SATMON019 cDNA library is generated from maize (DK604, Dekalb Genetics, Dekalb, Ill. U.S.A.) culm (stem) at the V8 developmental stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. When the maize plant is at the V8 stage, the 5th and 6th leaves from the bottom have fully developed leaf blades. The region between the nodes of the 5th and the sixth leaves from the bottom is the region of the stem that is collected. The leaves are pulled out and the sheath is also torn away from the stem. This stem tissue is completely free of any leaf and sheath tissue. The stem tissue is then frozen in liquid nitrogen and stored at −80° C. until RNA preparation. The RNA is purified from the stored tissue and the cDNA library is constructed as described in Example 2.

The SATMON020 cDNA library is from a maize (DK604, Dekalb Genetics, Dekalb, Ill. U.S.A.) Hill Type II-Initiated Callus. Petri plates containing approximately 25 ml of Type II initiation media are prepared. This medium contains N6 salts and vitamins, 3% sucrose, 2.3 g/liter proline 0.1 g/liter enzymatic casein hydrolysate, 2 mg/liter 2,4-dichloro phenoxy-acetic acid (2,4, D), 15.3 mg/liter AgNO₃ and 0.8% bacto agar and is adjusted to pH 6.0 before autoclaving. At 9-11 days after pollination, an ear with immature embryos measuring approximately 1-2 mm in length is chosen. The husks and silks are removed and then the ear is broken into halves and placed in an autoclaved solution of Clorox/TWEEN 20 sterilizing solution. Then the ear is rinsed with deionized water. Then each embryo is extracted from the kernel. Intact embryos are placed in contact with the medium, scutellar side up). Multiple embryos are plated on each plate and the plates are incubated in the dark at 25° C. Type II calluses are friable, can be subcultured with a spatula, frequently regenerate via somatic embryogenesis and are relatively undifferentiated. As seen in the microscope, the Tape II calluses show color ranging from translucent to light yellow and heterogeneity on with respect to embryoid structure as well as stage of embryoid development. Once Type II callus are formed, the calluses is transferred to type II callus maintenance medium without AgNO₃. Every 7-10 days, the callus is subcultured. About 4 weeks after embryo isolation the callus is removed from the plates and then frozen in liquid nitrogen. The harvested tissue is stored at −80° C. until RNA preparation. The RNA is purified from the stored tissue and the cDNA library is constructed as described in Example 2.

The SATMON021 cDNA library is generated from the immature maize (DK604, Dekalb Genetics, Dekalb Ill., U.S.A.) tassel at the V8 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. As the maize plant enters the V8 stage, tassels which are 15-20 cm in length are collected and frozen in liquid nitrogen. The harvested tissue is stored at −80° C. until RNA preparation. The RNA is purified from the stored tissue and the cDNA library is constructed as described in Example 2.

The SATMON022 cDNA library is generated from maize (DK604, Dekalb Genetics, Dekalb, Ill. U.S.A.) immature ear at the V8 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the plant is in the V8 stage. At this stage, some immature ear shoots are visible. The immature ear shoots (approximately 34 cm in length) are pulled out, frozen in liquid nitrogen and then stored at −80° C. until RNA preparation. The RNA is purified from the stored tissue and the cDNA library is constructed as described in Example 2.

The SATMON023 cDNA library is generated from maize (DK604, Dekalb Genetics, Dekalb, Ill. U.S.A.) ear (growing silk) at the V8 development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. When the tissue is harvested at the V8 stage, the length of the ear that is harvested is about 10-15 cm and the silks are just exposed (approximately 1 inch). The ear along with the silks is frozen in liquid nitrogen and then the tissue is stored at −80° C. until RNA preparation. The RNA is purified from the stored tissue and the cDNA library is constructed as described in Example 2.

The SATMON024 cDNA library is generated from the immature maize (DK604, Dekalb Genetics, Dekalb, Ill. U.S.A.) tassel at the V9 development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. As a maize plant enters the V9 stage, the tassel is rapidly developing and a 37 cm tassel along with the glume, anthers and pollen is collected and frozen in liquid nitrogen. The harvested tissue is stored at −80° C. until RNA preparation. The RNA is purified from the stored tissue and the cDNA library is constructed as described in Example 2.

The SATMON025 cDNA library is from maize (DK604, Dekalb Genetics, Dekalb, Ill. U.S.A.) Hill Type II-Regenerated Callus. Type II callus is grown in initiation media as described for SATMON020 and then the embryoids on the surface of the Type II callus are allowed to mature and germinate. The 1-2 gm fresh weight of the soft friable type callus containing numerous embryoids are transferred to 100×15 mm petri plates containing 25 ml of regeneration media. Regeneration media consists of Murashige and Skoog (MS) basal salts, modified White's vitamins (0.2 g/liter glycine and 0.5 g/liter myo-inositoland 0.8% bacto agar (6SMS0D)). The plates are then placed in the dark after covering with parafilm. After 1 week, the plates are moved to a lighted growth chamber with 16 hr light and 8 hr dark photoperiod. Three weeks after plating the Type II callus to 6SMS0D, the callus exhibit shoot formation. The callus and the shoots are transferred to fresh 6SMS0D plates for another 2 weeks. The callus and the shoots are then transferred to petri plates with reduced sucrose (3SMSOD). Upon distinct formation of a root and shoot, the newly developed green plants are then removed out with a spatula and frozen in liquid nitrogen containers. The harvested tissue is then stored at −80° C. until RNA preparation. The RNA is purified from the stored tissue and the cDNA library is constructed as described in Example 2.

The SATMON026 cDNA library is generated from maize (DK604, Dekalb Genetics, Dekalb, Ill. U.S.A.) juvenile/adult shift leaves at the V8 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plants are at the 8-leaf development stage. Leaves are founded sequentially around the meristem over weeks of time and the older, more juvenile leaves arise earlier and in a more basal position than the younger, more adult leaves, which are in a more apical position. In a V8 plant, some leaves which are in the middle portion of the plant exhibit characteristics of both juvenile as well as adult leaves. They exhibit a yellowing color but also exhibit, in part, a green color. These leaves are termed juvenile/adult shift leaves. The juvenile/adult shift leaves (the 4th, 5th leaves from the bottom) are cut at the base, pooled and transferred to liquid nitrogen in which they are then crushed. The harvested tissue is then stored at −80° C. until RNA preparation. The RNA is purified from the stored tissue and the cDNA library is constructed as described in Example 2.

The SATMON027 cDNA library is generated from maize (DK604, Dekalb Genetics, Dekalb, Ill. U.S.A.) leaves from plants at the V8 developmental stage that are subject to six days water stress. Seeds are planted at a depth of approximately 3 cm into 2-3 inch peat pots containing Metro 200 growing medium. After 2-3 weeks growth they are transplanted into 10 inch pots containing the Metro 200 growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Prior to tissue collection, when the plant is at the 8-leaf stage, water is held back for six days. The older, more juvenile leaves, which are in a basal position, as well as the younger, more adult leaves, which are more apical, are all cut at the base of the leaves. All the leaves exhibit significant wilting. The leaves are then pooled and immediately transferred to liquid nitrogen containers in which the pooled leaves are then crushed. The harvested tissue is then stored at −80° C. until RNA preparation. The RNA is purified from the stored tissue and the cDNA library is constructed as described in Example 2.

The SATMON028 cDNA library is generated from maize (DK604, Dekalb Genetics, Dekalb, Ill. U.S.A.) roots at the V8 developmental stage that are subject to six days water stress. Seeds are planted at a depth of approximately 3 cm into 2-3 inch peat pots containing Metro 200 growing medium. After 2-3 weeks growth they are transplanted into 10 inch pots containing the Metro 200 growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Prior to tissue collection, when the plant is at the 8-leaf stage, water is held back for six days. The root system is cut, shaken and washed to remove soil. Root tissue is then immediately transferred to liquid nitrogen containers. The harvested tissue is then stored at −80° C. until RNA preparation. The RNA is purified from the stored tissue and the cDNA library is constructed as described in Example 2.

The SATMON029 cDNA library is generated from maize (DK604, Dekalb Genetics, Dekalb, Ill. U.S.A.) seedlings at the etiolated stage. Seeds are planted on a moist filter paper on a covered tray that is kept in the dark for 4 days at approximately 70° F. Tissue is collected when the seedlings are 4 days old. By 4 days, the primary root has penetrated the coleorhiza and is about 4-5 cm and the secondary lateral roots have also made their appearance. The coleoptile has also pushed through the seed coat and is about 4-5 cm long. The seedlings are frozen in liquid nitrogen and crushed. The harvested tissue is then stored at −80° C. until RNA preparation The RNA is purified from the stored tissue and the cDNA library is constructed as described in Example 2.

The SATMON030 cDNA library is generated from maize (DK604, Dekalb Genetics, Dekalb, Ill. U.S.A.) root tissue at the V4 plant development stage. Seeds are planted at a depth of approximately 3 cm into 2-3 inch peat pots containing Metro 200 growing medium. After 2-3 weeks growth, they are transplanted into 10 inch pots containing the same. Plants are watered daily before transplantation and approximately 3 times a week after transplantation. Peters 15-16-17 fertilizer is applied approximately three times per week after transplanting, at a strength of 150 ppm N. Two to three times during the life time of the plant, from transplanting to flowering, a total of approximately 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 sodium vapor lamps. Tissue is collected when the maize plant is at the 4 leaf development stage. The root system is cut from the mature maize plant and washed with water to free it from the soil. The tissue is then immediately frozen in liquid nitrogen. The harvested tissue is then stored at −80° C. until RNA preparation. The RNA is purified from the stored tissue and the cDNA library is constructed as described in Example 2.

The SATMON031 cDNA library is generated from the maize (DK604, Dekalb Genetics, Dekalb, Ill. U.S.A.) leaf tissue at the V4 plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is 80° F. and the nighttime temperature is 70° F. Supplemental lighting is provided by 1000 W sodium vapor lamps. Tissue is collected when the maize plant is at the 4-leaf development stage. The third leaf from the bottom is cut at the base and immediately frozen in liquid nitrogen and crushed. The tissue is immediately frozen in liquid nitrogen. The harvested tissue is then stored at −80° C. until RNA preparation. The RNA is purified from the stored tissue and the cDNA library is constructed as described in Example 2.

The SATMON033 cDNA library is generated from maize (DK604, Dekalb Genetics, Dekalb, Ill. U.S.A.) embryo tissue from plants at 13 days after pollination. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. After the V10 stage, the ear shoots of the maize plant, which are ready for fertilization, are enclosed in a paper bag before silk emergent to withhold the pollen. The ear shoots are pollinated and 13 days after pollination, the ears are pulled out and then the kernels are plucked cut of the ears. Each kernel is then dissected into the embryo and the endosperm and the aleurone layer is removed. After dissection, the embryos are immediately frozen in liquid nitrogen and then stored at −80° C. until RNA preparation. The RNA is purified from the stored tissue and the cDNA library is constructed as described in Example 2.

The SATMON034 cDNA library is generated from cold stressed maize (DK604, Dekalb Genetics, Dekalb, Ill. U.S.A.) seedlings. Seeds are planted on a moist filter paper on a covered tray that is kept on at 10° C. for 7 days. After 7 days, the temperature is shifted to 15° C. for one day until germination of the seed. Tissue is collected once the seedlings are 1 day old. At this point, the coleorhiza has just pushed out of the seed coat and the primary root is just making its appearance. The coleoptile has not yet pushed completely through the seed coat and is also just making its appearance. These 1 day old cold stressed seedlings are frozen in liquid nitrogen and crushed. The harvested tissue is then stored at −80° C. until RNA preparation. The RNA is purified from the stored tissue and the cDNA library is constructed as described in Example 2.

The CMz029 (SATMON036) cDNA library is generated from maize (RX601, Asgrow Seed Company, Des Moines, Iowa U.S.A.) endosperm 22 days after pollination. RX601 corn seeds are sterilized for 1 minute in 10% clorox solution, rolled in germination paper and germinated in 0.5 mM Calcium Sufate for two days at 30° C. The seedlings are transplanted into a peat mix media in 3″ peat pots at the rate of three seedlings per pot. They are then placed in a greenhouse. Twenty plants are placed into a high CO₂ environment (˜1000 ppm CO₂) and twenty plants are grown under ambient greenhouse CO₂ (˜450 ppm CO₂). The plants are hand watered and lightly fertilized with Peters 20-20-20 liquid fertilizer. At 10 days after planting, the shoots from both atmospheres are placed in liquid nitrogen and lightly ground by hand. The roots are washed in DI water solution to remove most of the support media and then frozen in liquid nitrogen. All tissues are stored at −80° C. Shoots from the high CO₂ treatment are submitted for library preparation. The RNA is purified from the stored tissue and the cDNA library is constructed as described in Example 2.

The SATMONN04 normalized cDNA library is generated from maize (DK604, Dekalb Genetics, Dekalb, Ill. U.S.A.) embryo collected from plants at twenty one days after pollination. Seeds are planted at a depth of approximately 3 cm into 2-3 inch peat pots containing Metro 200 growing medium. After 2-3 weeks growth the seeds are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. After the V10 stage, the ear shoots of maize plant, which are ready for fertilization, are enclosed in a paper bag before silk emergence to withhold the pollen. The ear shoots are fertilized and 21 days after pollination, the ears are pulled out and the kernels are plucked out of the ears. Each kernel is then dissected into the embryo and the endosperm and the aleurone layer is removed. After dissection, the embryos are immediately frozen in liquid nitrogen and then stored at −80° C. until RNA preparation. The RNA is purified from the stored tissue and the cDNA library is constructed as described in Example 2. The library is normalized in one round using conditions adapted from Soares et al., Proc. Natl. Acad. Sci. (U.S.A.) 91:9928 (1994), the entirety of which is herein incorporated by reference and Bonaldo et al., Genome Res. 6: 791 (1996), the entirety of which is herein incorporated by reference except that a longer (48-hours/round) reannealing hybridization was used. SATMONN06 (normalized) and SATMON017 are embryo tissue libraries from the same donor.

The SATMONN05 cDNA library is a normalized library generated from maize (B73×Mo17, Illinois Foundation Seeds, Champaign Ill., U.S.A.) root tissue at the V6 development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is at the 6-leaf development stage. The root system is cut from the mature maize plant and washed with water to free it from the soil. The tissue is immediately frozen in liquid nitrogen and the harvested tissue is then stored at −80° C. until RNA preparation. The RNA is purified from the stored tissue. The library is normalized in two rounds using conditions adapted from Soares et al., Proc. Natl. Acad. Sci. (U.S.A.) 91:9928 (1994), the entirety of which is herein incorporated by reference and Bonaldo et al., Genome Res. 6: 791 (1996), the entirety of which is herein incorporated by reference except that a longer (48-hours/round) reannealing hybridization was used. SATMON003 is a root tissue library from the same donor.

The SATMONN06 normalized cDNA library is generated from maize (DK604, Dekalb Genetics, Dekalb, Ill. U.S.A.) embryo collected from plants at twenty one days after pollination. Seeds are planted at a depth of approximately 3 cm into 2-3 inch peat pots containing Metro 200 growing medium. After 2-3 weeks growth the seeds are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. After the V10 stage, the ear shoots of maize plant, which are ready for fertilization, are enclosed in a paper bag before silk emergence to withhold the pollen. The ear shoots are fertilized and 21 days after pollination, the ears are pulled out and the kernels are plucked out of the ears. Each kernel is then dissected into the embryo and the endosperm and the aleurone layer is removed. After dissection, the embryos are immediately frozen in liquid nitrogen and then stored at −80° C. until RNA preparation. The RNA is purified from the stored tissue and the cDNA library is constructed as described in Example 2. The library is normalized in two rounds using conditions adapted from Soares et al., Proc. Natl. Acad. Sci. (U.S.A.) 91:9928 (1994), the entirety of which is herein incorporated by reference and Bonaldo et al., Genome Res. 6: 791 (1996), the entirety of which is herein incorporated by reference except that a longer (48-hours/round) reannealing hybridization was used. SATMONN04 (normalized) and SATMON017 are embryo tissue libraries from the same donor.

LIB36 is a normalized cDNA library prepared from maize (DK604, Dekalb Genetics, Dekalb, Ill. U.S.A) leaves harvested from V8 stage plants. Seeds are planted at a depth of approximately 3 cm in soil into 2″-3″ peat pots containing Metro 200 growing medium. After 2-3 weeks growth, they are transplanted into 10″ pots containing the same. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is 80° F. and the nighttime temperature is 70° F. Lighting is provided by 1000 W sodium vapor lamps. Tissue is collected from V8 stage plants. The older more juvenile leaves which are in a basal position as well as the younger more adult leaves which are more apical were all cut at the base of the leaves. The leaves are then pooled and then immediately transferred to liquid nitrogen containers in which the pooled leaves are then crushed. The harvested tissue is then stored at −80° C. until RNA preparation.

For the construction of a cDNA library, the Superscript™ Plasmid System for cDNA synthesis and Plasmid Cloning (Gibco BRL, Life Technologies, Gaithersburg, Md.) or similar system, following the conditions suggested by the manufacturer, is used. Poly A+ mRNA is purified from the total RNA preparation using Dynabeads® Oligo (dT)₂₅ (Dynal Inc., Lake Success, N.Y.), or equivalent methods. Clones are selected and the plasmid DNA is isolated using a commercially available kit for normalizing the cDNA library. This library is normalized at a cot value of 10.

Approximately 1 million clones from the cDNA library are used for the generation of double and single stranded plasmid DNA. Double stranded plasmid DNA is used as a template for preparation of biotinylated RNA transcripts. Single stranded plasmid DNA from the cDNA library is hybridized with biotinylated RNA transcripts from the same library. Hybridized molecules are removed with Streptavidin beads (Dynal Inc. Lake Success, N.Y.). Remaining single stranded molecules are partially repaired with “Klenow” before transforming E. coli for the generation of a normalized cDNA library. SATMON009 is a leaf tissue library from the same donor.

The normalized cDNA library (LIB83) is prepared from maize leaves harvested from V8 stage plants. Maize DK604 (Dekalb Genetics, Dekalb, Ill. U.S.A) is used. Seeds are planted at a depth of approximately 3 cm in soil into 2″-3″ peat pots containing Metro 200 growing medium. After 2-3 weeks growth, they are transplanted into 10″ pots containing the same. Plants are watered daily before transplantation and ˜3 times a week after transplantation. Peters 15-16-17 fertilizer is applied ˜3× per week after transplanting, at a strength of 150 ppm N. 2-3 times during the life time of the plant, from transplanting to flowering, a total of ˜900 mg Fe is added to each pot. Plants are grown in a green house in 15 hr day/9 hr night cycles. The daytime temperature is 80° F. and the night time temperature was 70° F. Lighting was provided by 1000 W sodium vapor lamps. Tissue is collected from V8 stage plants. The older more juvenile leaves which are in a basal position as well as the younger more adult leaves which are more apical were all cut at the base of the leaves. The leaves are then pooled and then immediately transferred to liquid nitrogen containers in which the pooled leaves are then crushed. The harvested tissue is then stored at −80° C. until RNA preparation.

For the construction of a cDNA library, the Superscript™ Plasmid System for cDNA synthesis and Plasmid Cloning (Gibco BRL, Life Technologies, Gaithersburg, Md.) or similar system, following the conditions suggested by the manufacturer, is used. Poly A+ mRNA is purified from the total RNA preparation using Dynabeads® Oligo (dT)₂₅ (Dynal Inc., Lake Success, N.Y.), or equivalent methods. Clones are selected and the plasmid DNA is isolated using a commercially available kit for normalizing the cDNA library. This library is normalized at a ratio of 1:50.

Approximately 1 million clones from the cDNA library are used for the generation of double and single stranded plasmid DNA. Double stranded plasmid DNA is used as a template for preparation of biotinylated RNA transcripts. Single stranded plasmid DNA from the cDNA library is hybridized with biotinylated RNA transcripts from the same library. Hybridized molecules are removed with Streptavidin beads (Dynal Inc. Lake Success, N.Y.). Remaining single stranded molecules are partially repaired with “Klenow” before transforming E. coli for the generation of a normalized cDNA library. SATMON009 is a leaf tissue library from the same donor.

LIB84 a normalized cDNA library is prepared from maize (DK604, Dekalb Genetics, Dekalb, Ill. U.S.A) leaves harvested from V8 stage plants. Seeds are planted at a depth of approximately 3 cm in soil into 2″-3″ peat pots containing Metro 200 growing medium. After 2-3 weeks growth, they are transplanted into 10″ pots containing the same. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Plants were grown in a greenhouse in 15 hr day/9 hr night cycles. The daytime temperature was 80° F. and the nighttime temperature was 70° F. Lighting was provided by 1000 W sodium vapor lamps. Tissue was collected from V8 stage plants. The older more juvenile leaves which are in a basal position as well as the younger more adult leaves which are more apical were all cut at the base of the leaves. The leaves are then pooled and then immediately transferred to liquid nitrogen containers in which the pooled leaves are then crushed. The harvested tissue is then stored at −80° C. until RNA preparation.

For the construction of a cDNA library, the Superscript™ Plasmid System for cDNA synthesis and Plasmid Cloning (Gibco BRL, Life Technologies, Gaithersburg, Md.) or similar system, following the conditions suggested by the manufacturer, is used. Poly A+ mRNA is purified from the total RNA preparation using Dynabeads® Oligo (dT)₂₅ (Dynal Inc., Lake Success, N.Y.), or equivalent methods. Clones are selected and the plasmid DNA is isolated using a commercially available kit for normalizing the cDNA library. This library is normalized at a ratio of 1:10.

Approximately 1 million clones from the cDNA library are used for the generation of single stranded plasmid DNA. Appropriate Oligonucleotide from 3′ end of single stranded circle are used for primer extension in the presence of biotinylated dideoxynucleotides. The reaction is controlled to give 200-300 bp extension products. Single stranded circle cDNA library with primer extension products is denatured and hybridized under appropriate conditions. Hybridized molecules are removed with Streptavidin beads (Dynal Inc. Lake Success, N.Y.). Remaining single stranded molecules are partially repaired with “Klenow” before transforming E. coli for the generation of a normalized cDNA library. SATMON009 is a leaf tissue library from the same donor.

The CMz030 (Lib143) cDNA library is generated from maize (DK604, Dekalb Genetics, Dekalb, Ill. U.S.A.) heat shocked seedling tissue two days post germination. Seeds are planted on a moist filter paper on a covered try that is keep in the dark until germination. The trays are then moved to the bench top at 15 hr daytime/9 hr nighttime cycles for 2 days post-germination. The day time temperature is 80° F. and the nighttime temperature is 70° F. Tissue is collected when the seedlings are 2 days old. At this stage, the colehrhiza has pushed through the seed coat and the primary root (the radicle) is just piercing the colehrhiza and is barely visible. The seedlings are placed at 42° C. for 1 hour. Following the heat shock treatment, the seedlings are immersed in liquid nitrogen and crushed. The harvested tissue is stored at −80° until RNA preparation. The RNA is purified from the stored tissue and the cDNA library is constructed as described in Example 2.

The CMz031 (Lib148) cDNA library is generated from maize (DK604, Dekalb Genetics, Dekalb, Ill. U.S.A.) pollen tissue at the V10+ plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from V10+ stage plants. The ear shoots, which are ready for fertilization, are enclosed in a paper bag to withhold pollen. Twenty-one days after pollination, prior to removing the ears, the paper bag is shaken to collect the mature pollen. The mature pollen is immediately frozen in liquid nitrogen containers and the pollen is crushed. The harvested tissue is then stored at −80° C. until RNA preparation. The RNA is purified from the stored tissue and the cDNA library is constructed as described in Example 2.

The CMz033 (Lib189) cDNA library is generated from maize (RX601, Asgrow Seed Company, Des Moines, Iowa U.S.A.) pooled leaf tissue harvested from field grown plants at Asgrow research stations. Leaves are harvested at anthesis from open pollinated plants in a field (multiple row) setting. The ear leaves from 10-12 plants are harvested, pooled, frozen in liquid nitrogen and then frozen at −80° C. where they are stored until RNA preparation. The RNA is purified from the stored tissue and the cDNA library is constructed as described in Example 2.

The CMz034 (Lib3060) cDNA library is generated from maize (DK604, Dekalb Genetics, Dekalb, Ill. U.S.A.) senescing leaves from plants at 40 days after pollination. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from leaves located two leaves below the ear leaf. This sample represents those genes expressed during onset and early stages of leaf senescence. The leaves are pooled and immediately transferred to liquid nitrogen. The harvested tissue is then stored at −80° C. until RNA preparation. The RNA is purified from the stored tissue and the cDNA library is constructed as described in Example 2.

The CMz035 (Lib3061) cDNA library is generated from maize (DK604, Dekalb Genetics, Dekalb, Ill. U.S.A.) endosperm tissue from plants at 32 days after pollination. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from V10+ stage plants. The corn plant is beyond the V10 stage and the ear shoots, which are ready for fertilization, are enclosed in a paper bag prior to silk emergence to withhold pollen. Thirty-two days after pollination, the ears are pulled out and the kernels are removed from the cob. Each kernel is dissected into the embryo and the endosperm and the aleurone layer is removed. After dissection, the endosperms are immediately transferred to liquid nitrogen. The harvested tissue is then stored at 80° C. until RNA preparation. The RNA is purified from the stored tissue and the cDNA library is constructed as described in Example 2.

The CMz036 (Lib3062) cDNA library is generated from maize (H99, USDA Regional Plant Introduction Station, Ames, Iowa U.S.A.) husk tissue from 8 week old plants. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from 8 week old plants. The husk is separated from the ear and immediately transferred to liquid nitrogen containers. The harvested tissue is then stored at −80° C. until RNA preparation. The RNA is purified from the stored tissue and the cDNA library is constructed as described in Example 2.

The CMz037 (Lib3059) cDNA library is generated from maize (RX601, Asgrow Seed Company, Des Moines, Iowa U.S.A) pooled kernels from plants at 12-15 days after pollination. Sample are collected from field grown material. Whole kernels from hand pollinated (control pollination) are harvested as whole ears and immediately frozen on dry ice. Kernels from 10-12 ears are pooled and ground together in liquid nitrogen. The harvested tissue is then stored at −80° C. until RNA preparation. The RNA is purified from the stored tissue and the cDNA library is constructed as described in Example 2.

The CMz039 (Lib3066) cDNA library is generated from maize (H99, USDA Regional Plant Introduction Station, Ames, Iowa U.S.A.) immature anther tissue at the 7 week old immature tassel stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is at the 7 week old immature tassel stage. At this stage, prior to anthesis, the immature anthers are green and enclosed in the staminate spikelet. The developing anthers are dissected away from the 7 week old immature tassel and immediately frozen in liquid nitrogen. The harvested tissue is then stored at −80° C. until RNA preparation. The RNA is purified from the stored tissue and the cDNA library is constructed as described in Example 2.

The CMz040 (Lib3067) cDNA library is generated from maize (MO17, USDA Regional Plant Introduction Station, Ames, Iowa U.S.A.) kernel tissue from plants at the V10+ plant development stage, 5-8 days after pollination. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from V10+ stage plants. The ear shoots, which are ready for fertilization, are enclosed in a paper bag before silk emergence to withhold pollen. Five to eight days after controlled pollination, the ears are pulled and the kernels removed. The kernels are immediately frozen in liquid nitrogen. This sample represents genes expressed in early kernel development, during periods of cell division, amyloplast biogenesis and early carbon flow across the material to filial tissue. The harvested kernels tissue is then stored at −80° C. until RNA preparation. The RNA is purified from the stored tissue and the cDNA library is constructed as described in Example 2.

The CMz041 (Lib3068) cDNA library is generated from maize pollen germinating silk tissue from plants at the V10+ plant development stage. Maize M017 and H99 (USDA Regional Plant Introduction Station, Ames, Iowa U.S.A.) seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from V10+ stage plants when the ear shoots are ready for fertilization at the silk emergence stage. The H99 emerging silks are pollinated with an excess of MO17 pollen under controlled pollination conditions in the greenhouse. Eighteen hours after pollination the silks are removed from the ears and immediately frozen in liquid nitrogen. This sample represents genes expressed in both pollen and silk tissue early in pollination. The harvested tissue is then stored at −80° C. until RNA preparation. The RNA is purified from the stored tissue and the cDNA library is constructed as described in Example 2.

The CMz042 (Lib3069) cDNA library is generated from maize ear tissue excessively pollinated at the V10+ plant development stage. Maize M017 and H99 (USDA Regional Plant Introduction Station, Ames, Iowa U.S.A.) seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from V10+ stage plants and the ear shoots which are ready for fertilization are at the silk emergence stage. The H99 immature ears are pollinated with an excess of MO17 pollen under controlled pollination conditions. Eighteen hours post-pollination, the ears are removed and immediately transferred to liquid nitrogen containers. The harvested tissue is then stored at −80° C. until RNA preparation. The RNA is purified from the stored tissue and the cDNA library is constructed as described in Example 2.

The CMz044 (Lib3075) cDNA library is generated from maize (H99, USDA Regional Plant Introduction Station, Ames, Iowa U.S.A.) microspore tissue. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from immature anthers from 7 week old tassels. The immature anthers are first dissected from the 7 week old tassel with a scalpel on a glass slide covered with water. The microspores (immature pollen) are released into the water and are recovered by centrifugation. The microspore suspension is immediately frozen in liquid nitrogen. The harvested tissue is then stored at −80° C. until RNA preparation. The RNA is purified from the stored tissue and the cDNA library is constructed as described in Example 2.

The CMz045 (Lib3076) cDNA library is generated from maize (H99, USDA Regional Plant Introduction Station, Ames, Iowa U.S.A.) immature ear megaspore tissue. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. The immature ears are harvested from the 7 week old plants and are approximately 2.5 to 3 cm in length. The kernels are removed from the cob and immediately frozen in liquid nitrogen. The harvested tissue is then stored at −80° C. until RNA preparation. The RNA is purified from the stored tissue and the cDNA library is constructed as described in Example 2.

The CMz047 (Lib3078) cDNA library is generated from maize (RX601, Asgrow Seed Company, Des Moines, Iowa, U.S.A.) CO₂ treated high-exposure shoot tissue. RX601 maize seeds are sterilized for 1 minute with a 10% Clorox solution. The seeds are rolled in germination paper, and germinated in 0.5 mM calcium sulfate solution for two days at 30° C. The seedlings are transplanted into a peat mix media in 3″ peat pots at the rate of three seedlings per pot. They are then placed in a greenhouse. Twenty pots are placed into a high CO₂ environment (approximately 1000 ppm CO₂). Twenty plants are grown under ambient greenhouse CO₂ (approximately 450 ppm CO₂). Plants are hand watered. Peters 20-20-20 fertilizer is also lightly applied. Maize plants are grown in a greenhouse in 15 hr day/9 hr night cycles. At ten days post planting, the shoots from both atmospheres are frozen in liquid nitrogen and lightly ground by hand. The roots are washed in deionized water to remove the support media and the tissue is immediately transferred to liquid nitrogen containers. The harvested tissue is then stored at −80° C. until RNA preparation. The RNA is purified from the stored tissue and the cDNA library is constructed as described in Example 2.

The CMz048 (Lib3079) cDNA library is generated from maize (MO17, USDA Maize Regional Plant Introduction Station, Ames, Iowa U.S.A) basal endosperm transfer layer tissue. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected from V10+ maize plants. The ear shoots, which are ready for fertilization, are enclosed in a paper bag prior to silk emergence, to withhold the pollen. Kernels are harvested at 12 days post-pollination and placed on wet ice for dissection. The kernels are cross sectioned laterally, dissecting just above the pedicel region, including 1-2 mm of the lower endosperm and the basal endosperm transfer region. The pedicel and lower endosperm region containing the basal endosperm transfer layer is pooled and immediately frozen in liquid nitrogen. The harvested tissue is then stored at −80° C. until RNA preparation. The RNA is purified from the stored tissue and the cDNA library is constructed as described in Example 2.

The CMz049 (Lib3088) cDNA library is generated from maize (H99, USDA Maize Regional Plant Introduction Station, Ames, Iowa U.S.A) immature ear tissue from 8 weeks old plants. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Ears are harvested from 8 week old plants and are approximately 3.54.5 cm long. Kernels are dissected away from the cob, frozen in liquid nitrogen and stored at −80 C until preparation of RNA. The RNA is purified from the stored tissue and the cDNA library is constructed as described in Example 2.

The CMz050 (Lib3114) cDNA library is generated from silks from maize (B73, Illinois Foundation Seeds, Champaign, Ill. U.S.A.) plants at the V10+ plant development stage. Seeds are planted at a depth of approximately 3 cm into 2-3 inch peat pots containing Metro 200 growing medium. After 2-3 weeks growth they are transplanted into 10 inch pots containing the same growing medium. Plants are watered daily before transplantation and three times a week after transplantation. Peters 15-16-17 fertilizer is applied three times per week after transplanting at a strength of 150 ppm N. Two to three times during the lifetime of the plant, from transplanting to flowering, a total of 900 mg Fe is added to each pot. Maize plants are grown in a greenhouse in 15 hr day/9 hr night cycles. The daytime temperature is approximately 80° F. and the nighttime temperature is approximately 70° F. Supplemental lighting is provided by 1000 W sodium vapor lamps. Tissue is collected when the maize plant is beyond the V10 development stage and the ear shoots are approximately 15-20 cm in length. The ears are pulled and the silks are separated from the ears and immediately transferred to liquid nitrogen containers. The harvested tissue is then stored at −80° C. until RNA preparation. The RNA is purified from the stored tissue and the cDNA library is constructed as described in Example 2.

The SOYMON001 cDNA library is generated from soybean cultivar Asgrow 3244 (Asgrow Seed Company, Des Moines, Iowa U.S.A.) total leaf tissue at the V4 plant development stage. Leaf tissue from 38, field grown V4 stage plants is harvested from the 4^{th} node. Leaf tissue is removed from the plants and immediately frozen in dry-ice. The harvested tissue is then stored at −80° C. until RNA preparation. The RNA is purified from the stored tissue and the cDNA library is constructed as described in Example 2.

The SOYMON002 cDNA library is generated from soybean cultivar Asgrow 3244 (Asgrow Seed Company, Des Moines, Iowa U.S.A.) root tissue at the V4 plant development stage. Root tissue from 76, field grown V4 stage plants is harvested. The root systems is cut from the soybean plant and washed with water to free it from the soil and immediately frozen in dry-ice. The harvested tissue is then stored at −80° C. until RNA preparation. The RNA is purified from the stored tissue and the cDNA library is constructed as described in Example 2.

The SOYMON003 cDNA library is generated from soybean cultivar Asgrow 3244 (Asgrow Seed Company, Des Moines, Iowa U.S.A.) hypocotyl axis tissue from seedlings 2 day after-imbibition. Seeds are planted at a depth of approximately 2 cm into 2-3 inch peat pots containing Metromix 350 medium. Trays are placed in an environmental chamber and grown at 12 hr daytime/12 hr nighttime cycles. The daytime temperature is approximately 29° C. and the nighttime temperature approximately 24° C. Soil is checked and watered daily to maintain even moisture conditions. Tissue is collected 2 days after the start of imbibition. The 2 days after imbibition samples are separated into 3 collections after removal of any adhering seed coat. At 2 days after imbibition under the above conditions, the seedlings have significant expansion of the axis and are close to emerging from the soil. A few seedlings have cracked the soil surface and exhibited slight greening of the exposed cotyledons. The seedlings are washed in water to remove soil, hypocotyl axis harvested and immediately frozen in liquid nitrogen. The harvested tissue is then stored at −80° C. until RNA preparation. The RNA is purified from the stored tissue and the cDNA library is constructed as described in Example 2.

The SOYMON004 cDNA library is generated from soybean cultivar Asgrow 3244 (Asgrow Seed Company, Des Moines, Iowa U.S.A.) seedling cotyledon tissue harvested 2 day post-imbibition. Seeds are planted at a depth of approximately 2 cm into 2-3 inch peat pots containing Metromix 350 medium. Trays are placed in an environmental chamber and grown at 12 hr daytime/12 hr nighttime cycles. The daytime temperature is approximately 29° C. and the nighttime temperature approximately 24° C. Soil is checked and watered daily to maintain even moisture conditions. Tissue is collected 2 days after the start of imbibition. The 2 days after imbibition samples are separated into 3 collections after removal of any adhering seed coat. At 2 days after imbibition under the above conditions, the seedlings have significant expansion of the axis and are close to emerging from the soil. A few seedlings have cracked the soil surface and exhibited slight greening of the exposed cotyledons. The seedlings are washed in water to remove soil, cotyledons harvested and immediately frozen in liquid nitrogen. The harvested tissue is then stored at −80° C. until RNA preparation. The RNA is purified from the stored tissue and the cDNA library is constructed as described in Example 2.

The SOYMON005 cDNA library is generated from soybean cultivar Asgrow 3244 (Asgrow Seed Company, Des Moines, Iowa U.S.A.) hypocotyl axis tissue from seeds 6 hour post-imbibition. Seeds are planted at a depth of approximately 2 cm into 2-3 inch peat pots containing Metromix 350 medium. Trays are placed in an environmental chamber and grown at 12 hr daytime/12 hr nighttime cycles. The daytime temperature is approximately 29° C. and the nighttime temperature approximately 24° C. Soil is checked and watered daily to maintain even moisture conditions. Tissue is collected 6 hours after the start of imbibition. The 6 hours after imbibition sample is collected over the course of approximately 2 hours starting at 6 hours post imbibition. At the 6 hours after imbibition stage, not all cotyledons have become fully hydrated and germination. Radicle protrusion has not occurred. The seedlings are washed in water to remove soil, then the hypocotyl axis is harvested and immediately frozen in liquid nitrogen. The harvested tissue is then stored at −80° C. until RNA preparation. The RNA is purified from the stored tissue and the cDNA library is constructed as described in Example 2.

The SOYMON006 cDNA library is generated from soybean cultivar Asgrow 3244 (Asgrow Seed Company, Des Moines, Iowa U.S.A.) cotyledons from seeds 6 hour post-imbibition. Seeds are planted at a depth of approximately 2 cm into 2-3 inch peat pots containing Metromix 350 medium. Trays are placed in an environmental chamber and grown at 12 hr daytime/12 hr nighttime cycles. The daytime temperature is approximately 29° C. and the nighttime temperature approximately 24° C. Soil is checked and watered daily to maintain even moisture conditions. Tissue is collected 6 hours after imbibition. The 6 hours after imbibition sample is collected over the course of approximately 2 hours starting at 6 hours post-imbibition. At the 6 hours after imbibition, not all cotyledons have become fully hydrated and germination. Radicle protrusion has not occurred. The seedlings are washed in water to remove soil, then the cotyledon is harvested and immediately frozen in liquid nitrogen. The harvested tissue is then stored at −80° C. until RNA preparation. The RNA is purified from the stored tissue and the cDNA library is constructed as described in Example 2.

The SOYMON007 cDNA library is generated from soybean cultivar Asgrow 3244 (Asgrow Seed Company, Des Moines, Iowa U.S.A.) seed tissue. Seeds are harvested from plants grown in a field in Jerseyville 25 and 35 days after flowering. Seed pods are picked from all over the plant and the seeds are extracted from the pods. Approximately 4.4 g and 19.3 g of seeds are collected from the 25 and 35 days after flowering plants, respectively, placed into 14 ml polystyrene tubes and immediately immersed in dry ice. The harvested tissue is then stored at −80° C. until RNA preparation. Total RNA is prepared from the combination of 1.0 g and 3.4 g of seeds from 25 and 35 days after flowering plants and the cDNA library is constructed as described in Example 2.

The SOYMON008 cDNA library is generated from soybean cultivar Asgrow 3244 (Asgrow Seed Company, Des Moines, Iowa U.S.A.) leaf tissue harvested from 25 and 35 days post-flowering plants. Total leaf tissue is harvested from field grown plants. Approximately 19 g and 29 g of leaves are harvested from the fourth node of the plant 25 and 35 days post-flowering and immediately frozen in dry ice. The harvested tissue is then stored at −80° C. until RNA preparation. Total RNA is prepared from the combination of equal amounts of leaf tissue from both time points and the cDNA library is constructed as described in Example 2.

The SOYMON009 cDNA library is generated from soybean cutlivar C1944 (USDA Soybean Germplasm Collection, Urbana, Ill. U.S.A.) pod and seed tissue harvested 15 days post-flowering. Pods from field grown plants are harvested 15 days post-flowering. The pods are picked from all over the plant, placed into 14 ml polystyrene tubes and immediately immersed in dry-ice. Approximately 3 g of pod tissue is harvested. The harvested tissue is then stored at −80° C. until RNA preparation. The RNA is purified from the stored tissue and the cDNA library is constructed as described in Example 2.

The SOYMON010 cDNA library is generated from soybean cultivar C1944 (USDA Soybean Germplasm Collection, Urbana, Ill. U.S.A.) seed tissue harvested 40 days post-flowering. Pods from field grown plants are harvested 40 days post-flowering. The pods are picked from all over the plant. Pods and seeds are separated, approximately 19 g of seed tissue is harvested and immediately frozen in dry-ice. The harvested tissue is then stored at −80° C. until RNA preparation. The RNA is purified from the stored tissue and the cDNA library is constructed as described in Example 2.

The SOYMON011 cDNA library is generated from soybean cultivars Cristalina (USDA Soybean Germplasm Collection, Urbana, Ill. U.S.A.) and FT108 (Monsoy, Brazil) (tropical germ plasma) leaf tissue. Leaves are harvested from plants grown in an environmental chamber under 12 hr daytime/12 hr nighttime cycles. The daytime temperature is approximately 29° C. and the nighttime temperature approximately 24° C. Soil is checked and watered daily to maintain even moisture conditions. Approximately 30 g of leaves are harvested from the 4^{th} node of each of the Cristalina and FT108 cultivars and immediately frozen in dry ice. The harvested tissue is then stored at −80° C. until RNA preparation. Total RNA is prepared from the combination of equal amounts of leaf tissue from each cultivar and the cDNA library is constructed as described in Example 2.

The SOYMON012 cDNA library is generated from soybean cultivar Asgrow 3244 (Asgrow Seed Company, Des Moines, Iowa U.S.A.) leaf tissue. Leaves from field grown plants are harvested from the fourth node 15 days post-flowering. Approximately 12 g of leaves are harvested and immediately frozen in dry ice. The harvested tissue is then stored at −80° C. until RNA preparation. The RNA is purified from the stored tissue and the cDNA library is constructed as described in Example 2.

The SOYMON013 cDNA library is generated from soybean cultivar Asgrow 3244 (Asgrow Seed Company, Des Moines, Iowa U.S.A.) root and nodule tissue. Approximately 28 g of root tissue from field grown plants is harvested 15 days post-flowering. The root system is cut from the soybean plant, washed with water to free it from the soil and immediately frozen in dry-ice. The harvested tissue is then stored at −80° C. until RNA preparation. The RNA is purified from the stored tissue and the cDNA library is constructed as described in Example 2.

The SOYMON014 cDNA library is generated from soybean cultivar Asgrow 3244 (Asgrow Seed Company, Des Moines, Iowa U.S.A.) seeds and pods, which are harvested from plants grown in a field in Jerseyville 15 days after flowering. The pods are picked from all over the plant, placed into 14 ml polystyrene tubes and immediately immersed in dry-ice. Approximately 5 g of seeds are harvested. The harvested tissue is then stored at −80° C. until RNA preparation. The RNA is purified from the stored tissue and the cDNA library is constructed as described in Example 2.

The SOYMON015 cDNA is generated from soybean cultivar Asgrow 3244 (Asgrow Seed Company, Des Moines, Iowa U.S.A.) seed tissue harvested 45 and 55 days post-flowering. Seed pods from field grown plants are harvested 45 and 55 days after flowering. The seed pods are picked from all over the plant and the seeds extracted from the pods. Approximately 19 g and 31 g of seeds are harvested from the respective seed pods and immediately frozen in dry ice. The harvested tissue is then stored at −80° C. until RNA preparation. Total RNA is prepared from the combination of 0.75 g and 1.25 g of seeds from 45 and 55 days after flowering and the cDNA library is constructed as described in Example 2.

The SOYMON016 cDNA library is generated from soybean cultivar Asgrow 3244 (Asgrow Seed Company, Des Moines, Iowa U.S.A.) root tissue from plants grown in a field in Jerseyville. Field grown plants are uprooted and the roots quickly rinsed with water. The root tissue is then cut from the plants, placed immediately in 14 ml polystyrene tubes and immersed in dry-ice. Approximately, 61 g and 38 g of root tissue is harvested from the field grown plants 25 and 35 days post-flowering. The harvested tissue is then stored at −80° C. until RNA preparation. Total RNA is prepared from the combination of equal amounts of root tissue from both time points and the cDNA library is constructed as described in Example 2.

The SOYMON017 cDNA library is generated from soybean cultivar Asgrow 3244 (Asgrow Seed Company, Des Moines, Iowa U.S.A.) root tissue from plants grown in a field in Jersyville. The plants are uprooted and the roots quickly rinsed with water. The root tissue is then cut from the plants, placed immediately in 14 ml polystyrene tubes and immersed in dry-ice. The tissue is then transferred to a −80° C. freezer for storage. Approximately 28 g and 22 g of root tissue are harvested from field grown plants 45 and 55 days post-flowering. The harvested tissue is then stored at −80° C. until RNA preparation. Total RNA is prepared from the combination of equal amounts of root tissue from both time points and the cDNA library is constructed as described in Example 2.

The SOYMON018 cDNA is generated from soybean cultivar Asgrow 3244 (Asgrow Seed Company, Des Moines, Iowa U.S.A.) leaf tissue harvested from plants grown in a field in Jerseyville 45 and 55 days after flowering. Leaves from field grown plants are harvested 45 and 55 days after flowering from the fourth node. Approximately 27 g and 33 g of leaves are collected from the 45 and 55 days after flowering plants, placed into 14 ml polystyrene tubes and immediately immersed in dry ice. The harvested tissue is then stored at −80° C. until RNA preparation. Total RNA is prepared from the combination of equal amounts of leaf tissue from both time points and the cDNA library is constructed as described in Example 2.

The SOYMON019 cDNA library is generated from soybean cultivars Cristalina (USDA Soybean Germplasm Collection, Urbana, Ill. U.S.A.) and FT108 (Monsoy, Brazil) (tropical germ plasma) root tissue. Roots are harvested from plants grown in an environmental chamber under 12 hr daytime/12 hr nighttime cycles. The daytime temperature is approximately 29° C. and the nighttime temperature approximately 24° C. Soil is checked and watered daily to maintain even moisture conditions. Approximately 50 g and 56 g of roots are harvested from each of the Cristalina and F1108 cultivars and immediately frozen in dry ice. The plants are uprooted and the roots quickly rinsed in a pail of water. The root tissue is then cut from the plants, placed immediately in 14 ml polystyrene tubes and immersed in dry-ice. The harvested tissue is then stored at −80° C. until RNA preparation. Total RNA is prepared from the combination of equal amounts of root tissue from each cultivar and the cDNA library is constructed as described in Example 2.

The SOYMON020 cDNA is generated from soybean cultivar Asgrow 3244 (Asgrow Seed Company, Des Moines, Iowa U.S.A.) seeds harvested from plants grown in a field in Jerseyville 65 and 75 days post-flowering. The seed pods are picked from all over the plant and the seeds extracted from the pods. Approximately 14 g and 31 g of seeds are harvested from the respective seed pods and immediately frozen in dry ice. The harvested tissue is then stored at −80° C. until RNA preparation. Total RNA is prepared from the combination of equal numbers of seeds from 65 and 75 days after flowering and the cDNA library is constructed as described in Example 2.

The SOYMON021 cDNA library is generated from Soybean Cyst Nematode-resistant soybean cultivar Hartwig (USDA Soybean Germplasm Collection, Urbana, Ill. U.S.A.) root tissue. Plants are grown in tissue culture at room temperature. At approximately 6 weeks post-germination, the plants are exposed to sterilized Soybean Cyst Nematode eggs. Infection is then allowed to progress for 10 days. After the 10 day infection process, the tissue is harvested. Agar from the culture medium and nematodes are removed by blotting the root tissue on paper towels and then rinsing with water. The harvested root tissue is immediately frozen in dry ice and then stored at −80° C. until RNA preparation. The RNA is purified from the stored tissue and the cDNA library is constructed as described in Example 2.

The SOYMON022 cDNA library is generated from soybean cultivar Asgrow 3244 (Asgrow Seed Company, Des Moines, Iowa U.S.A.) partially to fully opened flower tissue, which is harvested from plants grown in an environmental chamber. Seeds are planted in moist Metromix 350 medium at a depth of approximately 2 cm. Trays are placed in an environmental chamber set to a 12 h day/12 h night cycle, 29° C. daytime temperature, 24° C. night temperature and 70% relative humidity. Daytime light levels are measured at 450 μEinsteins/m². Soil is checked and watered daily to maintain even moisture conditions. Flowers are removed from the plant at the pedicel. Flower buds showing petal color to fully open flowers are selected for collection. A total of 3 g of flower tissue is harvested and immediately frozen in dry ice. The harvested tissue is then stored at −80° C. until RNA preparation. Total RNA is prepared from a mixture of opened and partially opened flowers and the cDNA library is constructed as described in Example 2.

The SOYMON023 cDNA library is generated from soybean genotype BW211S Null (Tohoku University, Morioka, Japan) seed tissue harvested from plants grown in a field in Jerseyville. After 15 and 40 days, pods are harvested from all over the plant and seeds are dissected out from the pods. Approximately, 0.7 g and 14.2 g of seeds are harvested from the plants at the 15 and 40 days after flowering timepoints. The seeds are placed into 14 ml polystyrene tubes and immersed in dry-ice. The tissue is then transferred to a −80° C. freezer for storage. The harvested tissue is then stored at −80° C. until RNA preparation. Total RNA is prepared from the combination of 0.5 g and 1.0 g of seeds from the 15 and 40 days after flowering timepoints and the cDNA library is constructed as described in Example 2.

The SOYMON024 cDNA library is generated from soybean cultivar Asgrow 3244 (Asgrow Seed Company, Des Moines, Iowa U.S.A.) internode-2 tissue harvested 18 days post-imbibition. Seeds are planted at a depth of approximately 2 cm into 2-3 inch peat pots containing Metromix 350 medium. The plants are grown in a greenhouse for 18 days after the start of imbibition at ambient temperature. Stem tissue is harvested 18 days after the start of imbibition. The samples are divided into hypocotyl and internodes 1 through 5. The fifth internode contains some leaf bud material. Approximately 3 g of each sample is harvested and immediately frozen in dry ice. The harvested tissue is then stored at −80° C. until RNA preparation. Total RNA and poly A⁺ RNA is isolated from each sample as described in Example 2. One microgram of poly A⁺ RNA is electrophoresed on a denaturing 1% agarose gel and blotted by capillary transfer to a Nytran membrane using 20×SSC. The membrane is then UV crosslinked with 7.0×10⁴ μjoules/cm² and baked for 1 hour at 80° C. A probe consisting of the conserved core of the enzyme CPS (ent-kaurene synthetase) is radiolabeled with [α³²P]-dCTP and hybridized to the membrane overnight in Church buffer at 65° C. overnight. The blot is then washed 3 times in 1×SSC/0.1% SDS at 65° C. for 30 minutes per wash. This is used to determine which internode has the highest level of CPS expression and would thus be active in giberellic acid synthesis. After hybridization and washing the blot is exposed to a phosphorimager screen and exposed overnight. Processing of the image indicates that the highest level of CPS message could be found in internode 2. A library is constructed from internode 2 poly A⁺ RNA as described in Example 2.

The SOYMON025 cDNA library is generated from soybean cultivar Asgrow 3244 (Asgrow Seed Company, Des Moines, Iowa U.S.A.) leaf tissue harvested 65 days post-flowering. Leaves are harvested from the fourth node of field grown plants 65 days post-flowering. Approximately 18.4 g of leaf tissue is harvested and immediately frozen in dry ice. The harvested tissue is then stored at −80° C. until RNA preparation. The RNA is purified from the stored tissue and the cDNA library is constructed as described in Example 2.

SOYMON026 cDNA library is generated from soybean cultivar Asgrow 3244 (Asgrow Seed Company, Des Moines, Iowa U.S.A.) root tissue harvested from plants grown in a field in Jersyville 65 and 75 days afterflowering. The plants are uprooted and the roots quickly rinsed with water. The root tissue is then cut from the plants, placed immediately in 14 ml polystyrene tubes and immersed in dry-ice. The tissue is then transferred to a −80° C. freezer for storage. Approximately 27 g and 40 g of root tissue from field grown plants is harvested 65 and 75 days after flowering. The harvested tissue is then stored at −80° C. until RNA preparation. Total RNA is prepared from the combination of equal amounts of root tissue from both time points and the cDNA library is constructed as described in Example 2.

The SOYMON027 cDNA library is generated from soybean cultivar Asgrow 3244 (Asgrow Seed Company, Des Moines, Iowa U.S.A.) pod tissue (without seeds) harvested from field grown plants 25 days post-flowering. Pods are picked from all over plants. Seeds are dissected from the pods and the seeds and pods are placed separately into 14 ml polystyrene tubes and immediately immersed in dry-ice. Approximately 17 g of seed pod tissue is harvested and immediately frozen in dry ice. The harvested tissue is then stored at −80° C. until RNA preparation. The RNA is purified from the stored tissue and the cDNA library is constructed as described in Example 2.

The SOYMON028 cDNA library is generated from soybean cultivar Asgrow 3244 (Asgrow Seed Company, Des Moines, Iowa U.S.A.) drought-stressed root tissue. Seeds are planted in moist Metromix 350 medium at a depth of approximately 2 cm in trays. The trays are placed in an environmental chamber set to a 12 h day/12 h night cycle, 26° C. daytime temperature, 21° C. night temperature and 70% relative humidity. Daytime light levels are measured at 300 μEinsteins/m². Soil is checked and watered daily to maintain even moisture conditions. At the R3 stage of development, water is withheld from half of the plant collection (drought stressed population). After 3 days, half of the plants from the drought stressed condition and half of the plants from the control population are harvested. After another 3 days (6 days post drought induction) the remaining plants are harvested. A total of 27 g and 40 g of root tissue is harvested from plants at two time points and immediately frozen in dry ice. The harvested tissue is then stored at −80° C. until RNA preparation. Total RNA is prepared from the combination of equal amounts of drought stressed root tissue from both time points and the cDNA library is constructed as described in Example 2.

The SOYMON029 cDNA library is generated from Soybean Cyst Nematode-resistant soybean cultivar PI07354 (USDA Soybean Germplasm Collection, Urbana, Ill. U.S.A.) root tissue. Late fall to early winter greenhouse grown plants are exposed to Soybean Cyst Nematode eggs. At 10 days post-infection, the plants are uprooted, rinsed briefly and the roots frozen in liquid nitrogen. Approximately 20 grams of root tissue is harvested from the infected plants. The harvested tissue is then stored at −80° C. until RNA preparation. The RNA is purified from the stored tissue and the cDNA library is constructed as described in Example 2.

The SOYMON030 cDNA library is generated from soybean cultivar Asgrow 3244 (Asgrow Seed Company, Des Moines, Iowa U.S.A.) flower bud tissue. Seeds are planted at a depth of approximately 2 cm into 2-3 inch peat pots containing Metromix 350 medium and the plants are grown in an environmental chamber under 12 hr daytime/12 hr nighttime cycles. The daytime temperature is approximately 29° C. and the nighttime temperature approximately 24° C. Soil is checked and watered daily to maintain even moisture conditions. Flower buds are removed from the plant at the pedicel. A total of 100 mg of flower buds are harvested and immediately frozen in liquid nitrogen. The harvested tissue is then stored at −80° C. until RNA preparation.Total RNA is prepared from 50 mg of tissue and used directly to generate a library using the Clontech SMART™ PCR cDNA (Clontech Laboratories, Palo Alto, Calif. (U.S.A.) library construction kit. The EcoRI/XhoI adaptors are used in this library construction. The cDNA is ligated into the pINCY vector.

The SOYMON031 cDNA library is generated from soybean cultivar Asgrow 3244 (Asgrow Seed Company, Des Moines, Iowa U.S.A.) carpel and stamen tissue. Seeds are planted at a depth of approximately 2 cm into 2-3 inch peat pots containing Metromix 350 medium and the plants are grown in an environmental chamber under 12 hr daytime/12 hr nighttime cycles. The daytime temperature is approximately 29° C. and the nighttime temperature approximately 24° C. Soil is checked and watered daily to maintain even moisture conditions. Flower buds are removed from the plant at the pedicel. Flowers are dissected to separate petals, sepals and reproductive structures (carpels and stamens). A total of 300 mg of carpel and stamen tissue are harvested and immediately frozen in liquid nitrogen. The harvested tissue is then stored at −80° C. until RNA preparation. Total RNA is prepared from 150 mg of tissue and used directly to generate a library using the Clontech SMART™ PCR cDNA (Clontech Laboratories, Palo Alto, Calif. (U.S.A.) library construction kit. The EcoRI/XhoI adaptors are used in this library construction. The cDNA is ligated into the pINCY vector.

The SOYMON032 cDNA library is prepared from the Asgrow cultivar A4922 (Asgrow Seed Company, Des Moines, Iowa U.S.A.) rehydrated dry soybean seed meristem tissue. Surface sterilized seeds are germinated in liquid media for 24 hours. The seed axis is then excised from the barely germinating seed, placed on tissue culture media and incubated overnight at 20° C. in the dark. The supportive tissue is removed from the explant prior to harvest. Approximately 570 mg of tissue is harvested and frozen in liquid nitrogen. The harvested tissue is then stored at −80° C. until RNA preparation. The RNA is purified from the stored tissue and the cDNA library is constructed as described in Example 2.

The SOYMON033 cDNA library is generated from soybean cultivar Asgrow 3244 (Asgrow Seed Company, Des Moines, Iowa U.S.A.) heat-shocked seedling tissue without cotyledons. Seeds are imbibed and germinated in vermiculite for 2 days under constant illumination (ca. 510 Lux). After 48 hours, the seedlings are transferred to an incubator set at 40° C. under constant illumination (ca. 560 Lux). After 30, 60 and 180 minutes seedlings are harvested and dissected. A portion of the seedling consisting of the root, hypocotyl and apical hook is frozen in liquid nitrogen and stored at −80° C. The seedlings after 2 days of imbibition are beginning to emerge from the vermiculite surface. The apical hooks are dark green in appearance. Total RNA and poly A⁺ RNA is prepared from equal amounts of pooled tissue. The RNA is purified from the stored tissue and the cDNA library is constructed as described in Example 2.

The SOYMON034 cDNA library is generated from soybean cultivar Asgrow 3244 (Asgrow Seed Company, Des Moines, Iowa U.S.A.) cold-shocked seedling tissue without cotyledons. Seeds are imbibed and germinated in vermiculite for 2 days under constant illumination (ca. 510 Lux). After 48 hours, the seedlings are transferred to a cold room set at 5° C. under constant illumination (ca. 560 Lux). After 30, 60 and 180 minutes seedlings are harvested and dissected. The seedlings after 2 days of imbibition are beginning to emerge from the vermiculite surface. The apical hooks are dark green in appearance. A portion of the seedling consisting of the root, hypocotyl and apical hook is frozen in liquid nitrogen and stored at −80° C. Total RNA is prepared from equal amounts of pooled tissue and the cDNA library is constructed as described in Example 2.

The SOYMON035 cDNA library is generated from soybean cultivar Asgrow 3244 (Asgrow Seed Company, Des Moines, Iowa U.S.A.) seed coat tissue. Seeds are planted at a depth of approximately 2 cm into 2-3 inch peat pots containing Metromix 350 medium and the plants are grown in an environmental chamber under 12 hr daytime/12 hr nighttime cycles. The daytime temperature is approximately 29° C. and the nighttime temperature 24° C. Soil is checked and watered daily to maintain even moisture conditions. Seeds are harvested from mid to nearly full maturation (seed coats are not yellowing). The entire embryo proper is removed from the seed coat sample and the seed coat tissue are harvested and immediately frozen in liquid nitrogen. The harvested tissue is then stored at −80° C. until RNA preparation. The RNA is purified from the stored tissue and the cDNA library is constructed as described in Example 2.

The SOYMON036 cDNA library is generated from soybean cultivars PI171451, P1227687 and P1229358 (USDA Soybean Germplasm Collection, Urbana, Ill. U.S.A.) insect challenged leaves. Plants from each of the three cultivars are grown in a screenhouse. The screenhouse is divided in half by a screen and one half of the screenhouse is infested with soybean looper and the other half infested with velvetbean caterpillar. A single leaf is taken from each of the representative plants at 3 different time points, 11 days after infestation, 2 weeks after infestation and 5 weeks after infestation and immediately frozen in liquid nitrogen. The harvested tissue is then stored at −80° C. until RNA preparation. Total RNA and poly A+ RNA is isolated from pooled tissue consisting of equal quantities of all 18 samples (3 genotypes×3 sample times×2 insect genotypes). The RNA is purified from the stored tissue and the cDNA library is constructed as described in Example 2.

The SOYMON037 cDNA library is generated from soybean cultivar A3244 (Asgrow Seed Company, Des Moines, Iowa U.S.A.) etiolated axis and radical tissue. Seeds are planted in moist vermiculite, wrapped and kept at room temperature in complete darkness until harvest. Etiolated axis and hypocotyl tissue is harvested at 2, 3 and 4 days post-planting. Samples are frozen in liquid nitrogen upon harvesting and stored at −80° C. until RNA preparation. 1 gram of each sample (axis+hypocotyl at day 2, 3 and 4) is pooled for RNA isolation. The RNA is purified from the pooled tissue and the cDNA library is constructed as described in Example 2.

The SOYMON038 cDNA library is generated from soybean variety Asgrow A3237 (Asgrow Seed Company, Des Moines, Iowa U.S.A.) rehydrated dry seeds. Explants are prepared for transformation after germination of surface-sterilized seeds on solid tissue media. After 6 days, at 28° C. and 18 hours of light per day, the germinated seeds are cold shocked at 4° C. for 24 hours. Meristemic tissue and part of the hypocotyl is remove and cotyledon excised. The prepared explant is then wounded for Agrobacterium infection. The 2 grams of harvested tissue is frozen in liquid nitrogen and stored at −80° C. until RNA preparation. The RNA is purified from the stored tissue and the cDNA library is constructed as described in Example 2.

The Soy51 (LIB3027) normalized cDNA library is prepared from SOYMON007, SOYMON015 and SOYMON020. Equal amounts of SOYMON007, SOYMON015, and SOYMON020 in the form of single stranded DNA, are mixed and used as the starting material for normalization.

Normalized libraries are made using essentially the Soares procedure (Soares et al., Proc. Natl. Acad. Sci. (U.S.A.) 91:9228-9232 (1994)). This approach is designed to reduce the initial 10,000-fold variation in individual cDNA frequencies to achieve abundances within one order of magnitude while maintaining the overall sequence complexity of the library. In the normalization process, the prevalence of high-abundance cDNA clones decreases dramatically, clones with mid-level abundance are relatively unaffected and clones for rare transcripts are effectively increased in abundance.

Normalized libraries are prepared from single-stranded DNA. Single-stranded DNA representing approximately 1×10⁶ colony forming units are isolated using standard protocols. RNA, complementary to the single-stranded DNA, is synthesized using the double stranded DNA as a template. Biotinylated dATP is incorporated into the RNA during the synthesis reaction. The single-stranded DNA is mixed with the biotinylated RNA in a 1:10 molar ratio) and allowed to hybridize. DNA-RNA hybrids are captured on Dynabeads M280 streptavidin (Dynabeads, Dynal Corporation, Lake Success, N.Y. U.S.A.). The dynabeads with captured hybrids are collected with a magnet. The non-hybridized single-stranded molecules remaining after hybrid capture are converted to double stranded form and represent the primary normalized library.

The Soy52 (LIB3028) normalized cDNA library is generated from Soy35 (SOYMON022). Single stranded DNA representing approximately 1×10⁶ colony forming units of Soy35 (SOYMON022) is used as the starting material for normalization. The Soares procedure (Soares et al., Proc. Natl. Acad. Sci. (U.S.A.) 91:9228-9232 (1994)) is used for normalization.

Normalized libraries are prepared from single-stranded DNA. Single-stranded DNA representing approximately 1×10⁶ colony forming units are isolated using standard protocols. RNA, complementary to the single-stranded DNA, is synthesized using the double stranded DNA as a template. Biotinylated dATP is incorporated into the RNA during the synthesis reaction. The single-stranded DNA is mixed with the biotinylated RNA in a 1:10 molar ratio) and allowed to hybridize. DNA-RNA hybrids are captured on Dynabeads M280 streptavidin (Dynabeads, Dynal Corporation, Lake Success, N.Y. U.S.A.). The dynabeads with captured hybrids are collected with a magnet. The non-hybridized single-stranded molecules remaining after hybrid capture are converted to double stranded form and represent the primary normalized library.

The Soy53 (LIB3039) cDNA library is generated from soybean cultivar Asgrow 3244 (Asgrow Seed Company, Des Moines, Iowa U.S.A.) seedling shoot apical meristem tissue. Seeds are planted at a depth of approximately 2 cm into 2-3 inch peat pots containing Metromix 350 medium and the plants are grown in an environmental chamber set to a 12 h day/12 h night cycle, 29° C. daytime temperature, 24° C. night temperature and 70% relative humidity. Daytime light levels are measured at 450 μEinsteins/m². Soil is checked and watered daily to maintain even moisture conditions. Apical tissue is harvested from seedling shoot meristem tissue, 7-8 days after the start of imbibition. The apex of each seedling is dissected to include the fifth node to the apical meristem. The fifth node corresponds to the third trifoliate leaf in the very early stages of development. Stipules completely envelop the leaf primordia at this time. A total of 200 mg of apical tissue is harvested and immediately frozen in liquid nitrogen. The harvested tissue is then stored at −80° C. until RNA preparation. Total RNA is prepared from 100 mg of tissue and used directly to generate a library using the Clonetech SMART PCR cDNA library construction kit. The cDNA generated by this method is ligated to SalI adaptors from the pSPORT cDNA system from Life Technologies for ligational insertion into the pSPORT vector.

The Soy54 (LIB3040) cDNA library is generated from soybean cultivar Asgrow 3244 (Asgrow Seed Company, Des Moines, Iowa U.S.A.) heart to torpedo stage embryo tissue. Seeds are planted at a depth of approximately 2 cm into 2-3 inch peat pots containing Metromix 350 medium and the plants are grown in an environmental chamber under 12 hr daytime/12 hr nighttime cycles. The daytime temperature is approximately 29° C. and the nighttime temperature 24° C. Soil is checked and watered daily to maintain even moisture conditions. Seeds are collected and embryos removed from surrounding endosperm and maternal tissues. Embryos from globular to young torpedo stages (by corresponding analogy to Arabidopsis) are collected with a bias towards the middle of this spectrum. Embryos which are beginning to show asymmetric development of cotyledons are considered the upper developmental boundary for the collection and are excluded. A total of 12 mg embryo tissue is frozen in liquid nitrogen. The harvested tissue is stored at −80° C. until RNA preparation. Total RNA is prepared from 12 mg of tissue and used directly to generate a library using the Clontech SMART™ PCR cDNA (Clontech Laboratories, Palo Alto, Calif. U.S.A.) library construction kit. The SalI adaptors are used in this library construction. The cDNA is ligated into the pSPORT vector.

Soy55 (LIB3049) cDNA library is generated from soybean cultivar Asgrow 3244 (Asgrow Seed Company, Des Moines, Iowa U.S.A.) young seed tissue. Seeds are planted at a depth of approximately 2 cm into 2-3 inch peat pots containing Metromix 350 medium and the plants are grown in an environmental chamber under 12 hr daytime/12 hr nighttime cycles. The daytime temperature is approximately 29° C. and the nighttime temperature 24° C. Soil is checked and watered daily to maintain even moisture conditions. Seeds are collected from very young pods (5 to 15 days after flowering). A total of 100 mg of seeds are harvested and frozen in liquid nitrogen. The harvested tissue is stored at −80° C. until RNA preparation. Total RNA is prepared from 100 mg of tissue and used directly to generate a library using the Clontech SMART™ PCR cDNA (Clontech Laboratories, Palo Alto, Calif. U.S.A.) library construction kit. The SalI adaptors are used in this library construction. The cDNA is ligated into the pSPORT vector.

Soy56 (LIB3029) cDNA library is prepared from Soy19 (SOYMON007), Soy27 (SOYMON015) and Soy33 (SOYMON020). Equal amounts of Soy19, Soy27 and Soy33, in the form of single stranded DNA, are mixed in equimolar quantities. This mixture is used as the starting material for construction of the cDNA library and as a non-normalized control for comparison to Soy51. The cDNA library is constructed as described in Example 2.

The Soy57 (LIB3030) cDNA library is generated from soybean cultivar Asgrow 3244 (Asgrow Seed Company, Des Moines, Iowa U.S.A.) partially to fully opened flower tissue, which is harvested from plants grown in an environmental chamber. Seeds are planted in moist Metromix 350 medium at a depth of approximately 2 cm. Trays are placed in an environmental chamber set to a 12 h day/12 h night cycle, 29° C. daytime temperature, 24° C. night temperature and 70% relative humidity. Daytime light levels are measured at 450 μEinsteins/m². Soil is checked and watered daily to maintain even moisture conditions. Flowers are removed from the plant at the pedicel. Flower buds showing petal color to fully open flowers are selected for collection. A total of 3 g of flower tissue is harvested and immediately frozen in dry ice. The harvested tissue is then stored at −80° C. until RNA preparation. Total RNA is prepared from a mixture of opened and partially opened flowers and the cDNA library is constructed as described in Example 2.

The Soy58 (LIB3050) cDNA library is generated by subtracting the target cDNA, which is prepared from soybean cultivar Asgrow 3244 (Asgrow Seed Company, Des Moines, Iowa U.S.A.) roots from drought stressed plants, from the driver cDNA, which is prepared from soybean cultivar Asgrow 3244 roots from non drought-stressed (control) plants. Seeds are planted at a depth of approximately 2 cm into 2-3 inch peat pots containing Metromix 350 medium and the plants are grown in an environmental chamber set to a 12 h day/12 h night cycle, 26° C. daytime temperature, 21° C. night temperature and 70% relative humidity. Daytime light levels are measured at 300 μEinsteins/m². Soil is checked and watered daily to maintain even moisture conditions. At the R3 stage of the plant drought is induced by withholding water. After 3 and 6 days root tissue from both drought stressed and control (watered regularly) plants are collected and frozen in dry-ice. The harvested tissue is stored at −80° C. until RNA preparation. The RNA is prepared from the stored tissue and cDNA libraries are constructed as described in Example 2. For subtraction, target cDNA is made from the drought stressed tissue total RNA using the SMART cDNA synthesis system from Clonetech. Driver first strand cDNA is covalently linked to Dynabeads following a protocol similar to that described in the Dynal literature. The target cDNA is then heat denatured and the second strand trapped using Dynabeads oligo-dt. The target second strand cDNA is then hybridized to the driver cDNA in 400 μl 2×SSPE for two rounds of hybridization at 65° C. and 20 hours. After each hybridization, the hybridization solution is removed from the system and the hybridized target cDNA removed from the driver by heat denaturation in water. The refreshed driver is then reintroduced to the hybridization for the next round of hybridization. After hybridization, the remaining cDNA is trapped with Dynabeads oligo-dT. The trapped cDNA is then amplified as in previous PCR based libraries and the resulting cDNA ligated into the pSPORT vector.

The Soy59 (LIB3051) cDNA library is generated from soybean cultivar Asgrow 3244 (Asgrow Seed Company, Des Moines, Iowa U.S.A.) endosperm tissue. Seeds are germinated on paper towels under laboratory ambient light conditions. At 8, 10 and 14 hours after imbibition, the seed coats are harvested. The endosperm consists of a very thin layer of tissue affixed to the inside of the seed coat. The seed coat and endosperm are frozen immediately after harvest in liquid nitrogen. The harvested tissue is stored at −80° C. until RNA preparation. The stored tissue is then used immediately for preparation of poly A+ RNA using Dynabeads oligo-dT in a direct isolation procedure described by the manufacturer. The cDNA library is constructed using the pSPORT cDNA synthesis kit from Life Technologies (Life Technologies, Gaithersburg, Md. U.S.A.). The resulting cDNA is ligated into the pSPORT.

The Soy60 (LIB3072) cDNA library is generated by subtracting the target cDNA, which is prepared from soybean cultivar Asgrow 3244 (Asgrow Seed Company, Des Moines, Iowa U.S.A.) seeds plus pods from drought stressed plants, from the driver cDNA, which is prepared from soybean cultivar Asgrow 3244 seeds plus pods from non drought-stressed (control) plants. Seeds are planted at a depth of approximately 2 cm into 2-3 inch peat pots containing Metromix 350 medium and the plants are grown in an environmental chamber set to a 12 h day/12 h night cycle, 26° C. daytime temperature, 21° C. nighttime temperature and 70% relative humidity. Daytime light levels are 300 μEinsteins/m². Soil is checked and watered daily to maintain even moisture conditions. At the R3 stage of the plant drought is induced by withholding water. After 3 and 6 days seeds and pods from both drought stressed and control (watered regularly) plants are collected from the fifth and sixth node and frozen in dry-ice. The harvested tissue is stored at −80° C. until RNA preparation. The RNA is prepared from the stored tissue as described in Example 2.

For subtraction, target cDNA is made from the drought stressed tissue total RNA using the SMART cDNA synthesis system from Clonetech. Driver first strand cDNA is covalently linked to Dynabeads following a protocol similar to that described in the Dynal literature. The target cDNA is then heat denatured and the second strand trapped using Dynabeads oligo-dT. The target second strand cDNA is then hybridized to the driver cDNA in 400 μl 4×SSPE for three rounds of hybridization at 65° C. and 20 hours. After each hybridization, the hybridization solution is removed from the system and the hybridized target cDNA removed from the driver by heat denaturation in water. The refreshed driver is then reintroduced to the hybridization for the next round of hybridization. After hybridization, the remaining cDNA is trapped with Dynabeads oligo-dT. The trapped cDNA is then amplified as in previous PCR based libraries and the resulting cDNA ligated into the pSPORT vector.

The Soy61 (LIB3073) cDNA library is generated by subtracting the target cDNA, which is prepared from soybean cultivar Asgrow 3244 (Asgrow Seed Company, Des Moines, Iowa U.S.A.) jasmonic acid treated seedling, from the driver cDNA, which is prepared from control buffer treated seedlings without cotyledon. Seeds are planted at a depth of approximately 2 cm into 2-3 inch peat pots containing Metromix 350 medium and the plants are grown in a greenhouse. The daytime temperature is approximately 29.4° C. and the nighttime temperature 20° C. Soil is checked and watered daily to maintain even moisture conditions. At 9 days post planting, the plantlets are sprayed with either control buffer of 0.1% Tween-20 or jasmonic acid (Sigma J-2500, Sigma, St. Louis, Mo. U.S.A.) at 1 mg/ml in 0.1% Tween-20. Plants are sprayed until runoff and the soil and the stem is soaked with the spraying solution. At 18 hours post application of jasmonic acid, the soybean plantlets appear growth retarded. After 18 hours, 24 hours and 48 hours post treatment, the cotyledons are removed and the remaining leaf and stem tissue above the soil is harvested and frozen in liquid nitrogen. The harvested tissue is stored at −80° C. until RNA preparation. To make RNA, the three sample timepoints are combined and ground. The RNA is prepared from the stored tissue as described in Example 2. For subtraction, target cDNA is made from the jasmonic acid treated tissue total RNA using the SMART cDNA synthesis system from Clonetech. Driver first strand cDNA from the control tissue is covalently linked to Dynabeads following a protocol similar to that described in the Dynal literature. The target cDNA is then heat denatured and the second strand trapped using Dynabeads oligo-dT. The target second strand cDNA is then hybridized to the driver cDNA in 400 μl 4×SSPE for three rounds of hybridization at 65° C. and 20 hours. After each hybridization, the hybridization solution is removed from the system and the hybridized target cDNA removed from the driver by heat denaturation in water. The refreshed driver is then reintroduced to the hybridization for the next round of hybridization. After hybridization, the remaining cDNA is trapped with Dynabeads oligo-dT. The trapped cDNA is then amplified as in previous PCR based libraries and the resulting cDNA ligated into the pSPORT vector. For this library's construction, the eighth fraction of the cDNA size fractionation step is used for ligation.

The Soy62 (LIB3074) cDNA library is generated by subtracting the target cDNA, which is prepared from soybean cultivar Asgrow 3244 (Asgrow Seed Company, Des Moines, Iowa U.S.A.) jasmonic acid treated seedlings without cotyledon, from the driver cDNA, which is prepared from soybean cultivar Asgrow 3244 control buffer treated seedlings without cotyledon. Seeds are planted at a depth of approximately 2 cm into 2-3 inch peat pots containing Metromix 350 medium and the plants are grown in a greenhouse. The daytime temperature is approximately 29.4° C. and the nighttime temperature 20° C. Soil is checked and watered daily to maintain even moisture conditions. At 9 days post planting, the plantlets are sprayed with either control buffer of 0.1% Tween-20 or jasmonic acid (Sigma J-2500, Sigma, St. Louis, Mo. U.S.A.) at 1 mg/ml in 0.1% Tween-20. Plants are sprayed until runoff and the soil and the stem is soaked with the spraying solution. At 18 hours post application of jasmonic acid, the soybean plantlets appear growth retarded. After 18 hours, 24 hours and 48 hours post treatment, the cotyledons are removed and the remaining leaf and stem tissue above the soil is harvested and frozen in liquid nitrogen. The harvested tissue is stored at −80° C. until RNA preparation. To make RNA, the three sample timepoints are combined and ground. The RNA is prepared from the stored tissue as described in Example 2. For subtraction, target cDNA is made from the jasmonic acid treated tissue total RNA using the SMART cDNA synthesis system from Clonetech. Driver first strand cDNA from the control tissue is covalently linked to Dynabeads following a protocol similar to that described in the Dynal literature. The target cDNA is then heat denatured and the second strand trapped using Dynabeads oligo-dT. The target second strand cDNA is then hybridized to the driver cDNA in 400 μl 4×SSPE for three rounds of hybridization at 65° C. and 20 hours. After each hybridization, the hybridization solution is removed from the system and the hybridized target cDNA removed from the driver by heat denaturation in water. The refreshed driver is then reintroduced to the hybridization for the next round of hybridization. After hybridization, the remaining cDNA is trapped with Dynabeads oligo-dT. The trapped cDNA is then amplified as in previous PCR based libraries and the resulting cDNA ligated into the pSPORT vector. For this library's construction, the ninth fraction of the cDNA size fractionation step is used for ligation.

The Soy65 (LIB3107) cDNA library is generated from soybean cultivar Asgrow 3244 (Asgrow Seed Company, Des Moines, Iowa U.S.A.) abscission zone tissue from drought-stressed plants. Seeds are planted at a depth of approximately 2 cm into 2-3 inch peat pots containing Metromix 350 medium and the plants are grown in an environmental chamber set to 12 h day/12 h night cycle; 26 degree C. daytime temperature, 21 degree C. night temperature; 70% relative humidity. Daytime light levels are measured at 300 microeinsteins per square meter. Plants are irrigated with 15-16-17 Peter's Mix. At the R3 stage of development, drought is imposed by withholding water. At 3, 4, 5 and 6 days, tissue is harvested and wilting is not obvious until the fourth day. Abscission layers from reproductive organs are harvested by cutting less than one millimeter proximal and distal to the layer. Immediately upon excision, samples are frozen in liquid nitrogen and are stored at −80° C. until RNA preparation. The following tissues are combined for the single library: four day stress, all nodes; 5 day stress, all nodes. The RNA is prepared from the stored tissue and the cDNA library is constructed as described in Example 2.

The Soy66 (LIB3109) cDNA library is generated from soybean cultivar Asgrow 3244 (Asgrow Seed Company, Des Moines, Iowa U.S.A.) abscission zone tissue from control (watered regularly) plants. Seeds are planted at a depth of approximately 2 cm into 2-3 inch peat pots containing Metromix 350 medium and the plants are grown in an environmental chamber set to 12 h day/12 h night cycle; 26 degree C. daytime temperature, 21 degree C. night temperature; 70% relative humidity. Daytime light levels are measured at 300 microeinsteins per square meter. Plants are irrigated with 15-16-17 Peter's Mix. At 3, 4, 5 and 6 days (relative to drought stress induction in plants for Soy65), abscission layer tissue is harvested. Abscission layers from reproductive organs are harvested by cutting less than one millimeter proximal and distal to the layer. Immediately upon excision samples are frozen in liquid nitrogen and stored at −80° C. until RNA preparation. The following samples are combined for this cDNA library: 4 day control, all nodes; 5 day control; all nodes. The RNA is prepared from the stored tissue and the cDNA library is constructed as described in Example 2.

Soy67 (LIB 3065) normalized cDNA library is prepared from SOYMON07, SOYMON015 and SOYMON020 prepared tissue. Equal amounts of Soy19 (SOYMON007), Soy27 (SOYMON015) and Soy33 (SOYMON020), in the form of double stranded DNA, are mixed and used as the starting material for normalization. For normalization, biotinylated genomic soybean DNA is used as the driver for the normalization reaction. Double stranded plasmid DNA representing approximately 1×10⁶ colony forming units is used as the target. The double stranded plasmid DNA is isolated using standard protocols. Approximately 4 micrograms of biotinylated genomic DNA is mixed with approximately 6 micrograms of double stranded plasmid DNA and allowed to hybridize. Genomic DNA-plasmid DNA hybrids are captured on Dynabeads M280 streptavidin. The dynabeads with captured hybrids are collected with a magnet. Captured hybrids are eluted in water. The resulting clones are subjected to a second round of hybridization identical to the first.

Soy68 (LIB3052) normalized cDNA library is prepared from SOYMON007, SOYMON015 and SOYMON020. Equal amounts of Soy19 (SOYMON007), Soy27 (SOYMON015) and Soy33 (SOYMON020), in the form of double stranded DNA, are mixed and used as the starting material for normalization. For normalization, biotinylated genomic soybean DNA is used as the driver for the normalization reaction. Double stranded plasmid DNA representing approximately 1×10⁶ colony forming units is used as the target. The double stranded plasmid DNA is isolated using standard protocols. Approximately 4 micrograms of biotinylated genomic DNA is mixed with approximately 6 micrograms of double stranded plasmid DNA and allowed to hybridize. Genomic DNA-plasmid DNA hybrids are captured on Dynabeads M280 streptavidin. The dynabeads with captured hybrids are collected with a magnet. Captured hybrids are eluted in water.

Soy69 (LIB3053) normalized cDNA library is generated from soybean cultivars Cristalina (USDA Soybean Germplasm Collection, Urbana, Ill. U.S.A.) and FT108 (Monsoy, Brazil, tropical germ plasma) normalized leaf tissue. Leaves are harvested from plants grown in an environmental chamber under 12 hr daytime/12 hr nighttime cycles. The daytime temperature is approximately 29° C. and the nighttime temperature approximately 24° C. Soil is checked and watered daily to maintain even moisture conditions. Approximately 30 g of leaves are harvested from the 4^{th} node of each of the Cristalina and FT108 cultivars and immediately frozen in dry ice. The harvested tissue is then stored at −80° C. until RNA preparation. Total RNA is prepared from the combination of equal amounts of leaf tissue from each cultivar and a cDNA library is constructed as described in Example 2. For normalization, approximately 1 million clones from the cDNA library are used for generation of double and single stranded plasmid DNA. Double stranded plasmid DNA is used as a template for preparation of biotinylated RNA transcripts. Single stranded plasmid DNA from the cDNA library is hybridized with biotinylated RNA transcripts from the same library. Hybridized molecules are removed with Streptavidin beads (Dynal Inc. Lake Success, N.Y.). Remaining single stranded molecules are partially repaired with “Klenow” before transforming E. coli for the generation of the normalized cDNA library.

LIB3054 is a normalized cDNA library generated from roots from two exotic soybean cultivars Cristilliana (USDA Soybean Germplasm Collection, Urbana, Ill. U.S.A.) and FT108 (Monsoy, Brazil, tropical germ plasma). The roots are harvested from plants grown an environmental chamber set to a 12 h day/12 h night cycle, 29° C. daytime temperature, 24° C. night temperature and 70% relative humidity. Daytime light levels are measured at 450Einsteins/m². Soil is checked and watered daily to maintain even moisture conditions. Approximately 50 g and 56 g of roots are collected from cultivar Cristilliana and cultivarFT108. The plants are uprooted and the roots quickly rinsed in a pail of water. The root tissue is then cut from the plants, placed immediately in 14 ml polystyrene tubes and immersed in dry-ice. The tissue is stored at −80° C. until RNA preparation. Total RNA is prepared from the combination of equal amounts of root tissue from each cultivar and a cDNA library is constructed as described in Example 2. For normalization, approximately 1 million clones from the cDNA library are used for generation of double and single stranded plasmid DNA. Double stranded plasmid DNA is used as a template for preparation of biotinylated RNA transcripts. Single stranded plasmid DNA from the cDNA library is hybridized with biotinylated RNA transcripts from the same library. Hybridized molecules are removed with Streptavidin beads (Dynal Inc. Lake Success, N.Y.). Remaining single stranded molecules are partially repaired with “Klenow” before transforming E. coli for the generation of the normalized cDNA library.

Soy70 (LIB3055) cDNA library is generated from soybean cultivars Cristalina (USDA Soybean Germplasm Collection, Urbana, Ill. U.S.A.) and FT108 (Monsoy, Brazil, tropical germ plasma) leaf tissue. Leaves are harvested from plants grown in an environmental chamber under 12 hr daytime/12 hr nighttime cycles. The daytime temperature is approximately 29° C. and the nighttime temperature approximately 24° C. Soil is checked and watered daily to maintain even moisture conditions. Approximately 30 g of leaves are harvested from the 4^{th} node of each of the Cristalina and FT108 cultivars and immediately frozen in dry ice. The harvested tissue is then stored at −80° C. until RNA preparation. Total RNA is prepared from the combination of equal amounts of leaf tissue from each cultivar and the cDNA library is constructed as described in Example 2.

Soy71 (LIB3056) cDNA library is generated from soybean cultivars Cristalina (USDA Soybean Germplasm Collection, Urbana, Ill. U.S.A.) and FT108 (Monsoy, Brazil, tropical germ plasma) root tissue. Roots are harvested from plants grown in an environmental chamber set to a 12 h day/12 h night cycle, 29° C. daytime temperature, 24° C. night temperature and 70% relative humidity. Daytime light levels are measured at 45 μEinsteins/m². Soil is checked and watered daily to maintain even moisture conditions. Approximately 50 g and 56 g of roots are harvested from cultivar Cristalina and cultivar FT108 and immediately frozen in dry ice. The harvested tissue is then stored at −80° C. until RNA preparation. Total RNA is prepared from the combination of equal amounts of root tissue from each cultivar and the cDNA library is constructed as described in Example 2.

LIB3087 cDNA library is generated from hypocotyl axis from soybean cultivar Asgrow 3244 (Asgrow Seed Company, Des Moines, Iowa U.S.A) seeds 4, 8 and 12 hours after imbibition. Seeds are imbibed in water for 4 hours at 30° C. and then the seed coat is removed. At the 4 hr timepoint axis tissue is immediately harvested and flash-frozen in liquid nitrogen. For 8 and 12 hr timepoints decoated seeds are transferred to cotton saturated with water and incubated at 30° C. for the remainder of the incubation period. Axis tissue is then excised and frozen in liquid nitrogen. Equal numbers of axes from each timepoint is pooled for RNA isolation. The collected tissue is stored at −80° C. Axis tissue consists of unexpanded root, hypocotyl, epicotyl and apex. The RNA is purified from the stored tissue and the cDNA library is constructed as described in Example 2.

LIB3092 (Soy75) cDNA library is generated by subtracting a target cDNA, which is prepared from soybean cultivar Asgrow 3244 (Asgrow Seed Company, Des Moines, Iowa U.S.A.) leaves from drought stressed plants, from a driver cDNA, which is prepared from leaves from control (watered regularly) plants. Seeds are planted in moist Metromix 350 medium at a depth of approximately 2 cm. Trays are placed in an environmental chamber set to a 12 h day/12 h night cycle, 26° C. daytime temperature, 21° C. night temperature and 70% relative humidity. Daytime light levels are measured at 300 mEinsteins/m². Soil is checked and watered daily to maintain even moisture conditions. At the R3 stage of the plant, drought is induced by withholding water. After 3 and 6 days tissue is harvested. Leaves from both drought stressed and control (watered regularly) plants are collected from the fifth and sixth node and frozen in dry-ice. The tissue is then transferred to a −80° C. freezer for storage. For subtraction, a standard cDNA library is constructed in the pSPORT vector. Driver first strand cDNA is covalently linked to Dynabeads following a protocol similar to that described in the Dynal literature. The target library is then heat denatured and hybridized to the driver cDNA in 400 ml 4×SSPE for five rounds of hybridization at 68° C. and 20 hours. After each hybridization, the hybridization solution is removed from the system and the hybridized target cDNA removed from the driver by heat denaturation in water. The refreshed driver is then reintroduced to the hybridization for the next round of hybridization. The remaining cDNA in the hybridization solution is then used to transform E. coli for sequencing.

Soy74 (LIB3093) cDNA library is generated from soybean cultivar Asgrow 3244 (Asgrow Seed Company, Des Moines, Iowa U.S.A.) leaves collected from control (watered regularly) plants. Seeds are planted at a depth of approximately 2 cm into 2-3 inch peat pots containing Metromix 350 medium and the plants are grown in an environmental chamber set to a 12 hr daytime/12 hr nighttime cycle, 26° C. daytime temperature, 21° C. night temperature and 70% relative humidity. Daytime light levels are measured at 300 μEinsteins/m². Soil is checked and watered daily to maintain even moisture conditions. At the R3 stage of the plant drought is induced by withholding water. After 3 and 6 days seeds and pods from both drought stressed and control (watered regularly) plants are collected from the fifth and sixth node and frozen in dry-ice. The harvested tissue from control plants is stored at −80° C. until RNA preparation. The RNA is purified from the stored control tissue and the cDNA library is constructed as described in Example 2.

The LIB3094 normalized cDNA library is generated from LIB3087. LIB3087 in the form of double-stranded plasmid DNA is used as the starting material for normalization. For normalization biotinylated genomic soybean DNA is used as the driver for the normalization reaction. Double stranded plasmid DNA representing approximately 1×10⁶ colony forming units is used as the target. The double stranded plasmid DNA is isolated using standard protocols. Approximately 4 micrograms of biotinylated genomic DNA is mixed with approximately 6 micrograms of double stranded plasmid DNA and allowed to hybridize. Genomic DNA-plasmid DNA hybrids are captured on Dynabeads M280 streptavidin. The dynabeads with captured hybrids are collected with a magnet. Captured hybrids are eluted in water. The resulting clones are subjected to a second round of hybridization identical to the first.

The Soy76 (Lib3106) cDNA library is generated from soybean cultivar Asgrow 3244 (Asgrow Seed Company, Des Moines, Iowa U.S.A.) jasmonic acid and arachidonic treated seedlings. Seeds are planted at a depth of approximately 2 cm into 2-3 inch peat pots containing Metromix 350 medium and the plants are grown in a greenhouse. The daytime temperature is approximately 29.4° C. and the nighttime temperature 20° C. Soil is checked and watered daily to maintain even moisture conditions. At 9 days post planting, the plantlets are sprayed with either control buffer of 0.1% Tween-20 or jasmonic acid (Sigma J-2500, Sigma, St. Louis, Mo. U.S.A.) at 1 mg/ml in 0.1% Tween-20. Plants are sprayed until runoff and the soil and the stem is soaked with the spraying solution. At 18 hours post application of jasmonic acid, the soybean plantlets appear growth retarded. Arachidonic acid treated seedlings are sprayed with 1 m/ml arachidonic acid in 0.1% Tween-20. After 18 hours, 24 hours and 48 hours post treatment, the cotyledons are removed and the remaining leaf and stem tissue above the soil is harvested and frozen in liquid nitrogen. The harvested tissue is stored at −80° C. until RNA preparation. To make RNA, the three sample timepoints from the jasmonic acid treated seedlings are combined and ground. RNA from the arachidonic acid treated seedlings is isolated separately. Poly A⁺RNA is extracted from each total RNA sample separately and combined to make a cDNA library using approximately equal amounts of mRNA from each treatment. For the construction of this cDNA library, fraction 10 of the size fractionated cDNA is ligated into the pSPORT vector (Invitrogen, Carlsbad Calif. U.S.A.) in order to capture some of the smaller transcripts characteristic of antifungal proteins.

Soy77 (LIB3108) cDNA library is generated from soybean cultivar Asgrow 3244 (Asgrow Seed Company, Des Moines, Iowa U.S.A.) control buffer (0.1% Tween-20) treated seedlings. Seeds are planted at a depth of approximately 2 cm into 2-3 inch peat pots containing Metromix 350 medium and the plants are grown in a greenhouse. The daytime temperature is approximately 29.4° C. and the nighttime temperature 20° C. Soil is checked and watered daily to maintain even moisture conditions. At 9 days post planting, the plantlets are sprayed with either control buffer of 0.1% Tween-20 or jasmonic acid (Sigma J-2500, Sigma, St. Louis, Mo. U.S.A.) at 1 mg/ml in 0.1% Tween-20. Plants are sprayed until runoff and the soil and the stem is soaked with the spraying solution. At 18 hours post application of jasmonic acid, the soybean plantlets appear growth retarded. After 18 hours, 24 hours and 48 hours post treatment, the cotyledons are removed and the remaining leaf and stem tissue above the soil is harvested and frozen in liquid nitrogen. The harvested tissue is stored at −80° C. until RNA preparation. To make RNA, the three sample timepoints from control buffer treated seedlings are combined and ground. The RNA is prepared from the stored tissue. For the construction of this cDNA library, fraction 10 of the size fractionated cDNA is ligated into the pSPORT vector in order to capture some of the smaller transcripts characteristic of antifungal proteins.

Soy72 (LIB3138) normalized cDNA library is generated from Soy5 (SOYMON001), Soy20 (SOYMON008) and Soy24 (SOYMON012), Soy28 (SOYMON018) and Soy38 (SOYMON025). Equal amounts of Soy5 (SOYMON001), Soy20 (SOYMON008), Soy24 (SOYMON012), Soy28 (SOYMON018) and Soy38 (SOYMON025) in the form of double stranded DNA are mixed and used as the starting material for normalization. Biotinylated genomic soybean DNA is used as the driver for the normalization reaction. Double stranded plasmid DNA representing approximately 1×10⁶ colony forming units is used as the target. The double stranded plasmid DNA is isolated using standard protocols. Approximately 4 micrograms of biotinylated genomic DNA is mixed with approximately 6 micrograms of double stranded plasmid DNA and allowed to hybridize. Genomic DNA-plasmid DNA hybrids are captured on Dynabeads M280 streptavidin. The dynabeads with captured hybrids are collected with a magnet. Captured hybrids are eluted in water. The resulting clones are subjected to a second round of hybridization identical to the first.

Soy73 (LIB3139) normalized cDNA library is generated from Soy6 (SOYMON002), Soy25 (SOYMON013) and Soy29 (SOYMON016), Soy31 (SOYMON017) and Soy39 (SOYMON026). Equal amounts of Soy6 (SOYMON002), Soy25 (SOYMON013) and Soy29 (SOYMON016), Soy31 (SOYMON017) and Soy39 (SOYMON026) in the form of double stranded DNA are mixed and used as the starting material for normalization. Biotinylated genomic soybean DNA is used as the driver for the normalization reaction. Double stranded plasmid DNA representing approximately 1×10⁶ colony forming units is used as the target. The double stranded plasmid DNA is isolated using standard protocols. Approximately 4 micrograms of biotinylated genomic DNA is mixed with approximately 6 micrograms of double stranded plasmid DNA and allowed to hybridize. Genomic DNA-plasmid DNA hybrids are captured on Dynabeads M280 streptavidin. The dynabeads with captured hybrids are collected with a magnet. Captured hybrids are eluted in water. The resulting clones are subjected to a second round of hybridization identical to the first.

### EXAMPLE 2

The stored RNA is purified using Trizol reagent from Life Technologies (Gibco BRL, Life Technologies, Gaithersburg, Md. U.S.A.), essentially as recommended by the manufacturer. Poly A+ RNA (mRNA) is purified using magnetic oligo dT beads essentially as recommended by the manufacturer (Dynabeads, Dynal Corporation, Lake Success, N.Y. U.S.A.).

Construction of plant cDNA libraries is well-known in the art and a number of cloning strategies exist. A number of cDNA library construction kits are commercially available. The Superscript™ Plasmid System for cDNA synthesis and Plasmid Cloning (Gibco BRL, Life Technologies, Gaithersburg, Md. U.S.A.) is used, following the conditions suggested by the manufacturer.

### EXAMPLE 3

The cDNA libraries are plated on LB agar containing the appropriate antibiotics for selection and incubated at 37° for a sufficient time to allow the growth of individual colonies. Single selective media colonies are individually placed in each well of a 96-well microtiter plates containing LB liquid including the selective antibiotics. The plates are incubated overnight at approximately 37° C. with gentle shaking to promote growth of the cultures. The plasmid DNA is isolated from each clone using Qiaprep plasmid isolation kits, using the conditions recommended by the manufacturer (Qiagen Inc., Santa Clara, Calif. U.S.A.).

Template plasmid DNA clones are used for subsequent sequencing. For sequencing, the ABI PRISM dRhodamine Terminator Cycle Sequencing Ready Reaction Kit with AmpliTaq® DNA Polymerase, FS, is used (PE Applied Biosystems, Foster City, Calif. U.S.A.).

### EXAMPLE 4

Nucleic acid sequences that encode for a protein are identified from the Monsanto EST PhytoSeq database using TBLASTN (default values)(TBLASTN compares a protein query against the six reading frames of a nucleic acid sequence). Matches found with BLAST P values equal or less than 0.001 (probability) or BLAST Score of equal or greater than 90 are classified as hits. If the program used to determine the hit is HMMSW then the score refers to HMMSW score.

In addition, the GenBank database is searched with BLASTN and BLASTX (default values) using ESTs as queries. EST that pass the hit probability threshold of 10e⁻⁸ for the following enzymes are combined with the hits generated by using TBLASTN (described above) and classified. Results from these searches are set forth in Table 1.

A cluster refers to a set of overlapping clones in the PhytoSeq database. Such an overlapping relationship among clones is designated as a “cluster” when BLAST scores from pairwise sequence comparisons of the member clones meets a predetermined minimum value or product score of 50 or more (Product Score=(BLAST SCORE×Percentage Identity)/(5× minimum [length (Seq1), length (Seq2)]))

Since clusters are formed on the basis of single-linkage relationships, it is possible for two non-overlapping clones to be members of the same cluster if, for instance, they both overlap a third clone with at least the predetermined minimum BLAST score (stringency). A cluster ID is arbitrarily assigned to all of those clones which belong to the same cluster at a given stringency and a particular clone will belong to only one cluster at a given stringency. If a cluster contains only a single clone (a “singleton”), then the cluster ID number will be negative, with an absolute value equal to the clone ID number of its single member. Clones grouped in a cluster in most cases represent a contiguous sequence.

### REFERENCES

The above references are incorporated in their entirety. In addition, these references, as well as each of those cited can be relied upon to make and use aspects of the invention.

Clone ID

The clone ID number refers to the particular clone in the PhytoSeq database. Each clone ID entry in the table refers to the clone whose sequence is used for (1) the sequence comparison whose scores are presented and/or (2) assignment to the particular cluster which is presented. Note that a clone may be included in this table even if its sequence comparison scores fail to meet the minimum standards for similarity. In such a case, the clone is included due solely to its association with a particular cluster for which sequences of one or more other member clones possess the required level of similarity.

Library

The library ID refers to the particular cDNA library from which a given clone is obtained. Each cDNA library is associated with the particular tissue(s), line(s) and developmental stage(s) from which it is isolated.

NCBI gi

Each sequence in the GenBank public database is arbitrarily assigned a unique NCBI gi (National Center for Biotechnology Information GenBank Identifier) number. In this table, the NCBI gi number which is associated (in the same row) with a given clone refers to the particular GenBank sequence which is used in the sequence comparison. This entry is omitted when a clone is included solely due to its association with a particular cluster.

Method

The entry in the “Method” column of the table refers to the type of BLAST search that is used for the sequence comparison. “CLUSTER” is entered when the sequence comparison scores for a given clone fail to meet the minimum values required for significant similarity. In such cases, the clone is listed in the table solely as a result of its association with a given cluster for which sequences of one or more other member clones possess the required level of similarity.

Score

Each entry in the “Score” column of the table refers to the BLAST score that is generated by sequence comparison of the designated clone with the designated GenBank sequence using the designated BLAST method. This entry is omitted when a clone is included solely due to its association with a particular cluster. If the program used to determine the hit is HMMSW then the score refers to HMMSW score.

P-Value

The entries in the P-Value column refer to the probability that such matches occur by chance.

% Ident

The entries in the “% Ident” column of the table refer to the percentage of identically matched nucleotides (or residues) that exist along the length of that portion of the sequences which is aligned by the BLAST comparison to generate the statistical scores presented. This entry is omitted when a clone is included solely due to its association with a particular cluster.

### DESCRIPTION

The entries in the “Description” column of the table refer to the description associated with the NCBI gI number in the GenBank public database.

LENGTHY TABLE REFERENCED HERE
US20080034453A1-20080207-T00001
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20080034453A1). An electronic copy of the table will also be available from the USPTO upon request and payment of the fee set forth in 37 CFR 1.19(b)(3).

We claim:

1. A substantially purified nucleic acid molecule comprising nucleic acid sequence SEQ ID NO: 51419 or complement thereof or fragment of either.

2. A substantially purified first nucleic acid molecule, wherein the first nucleic acid molecule specifically hybridizes to a second nucleic acid molecule having a nucleic acid sequence SEQ ID NO: 51419 or complement thereof.

3. A substantially purified protein or fragment thereof encoded by a first nucleic acid molecule which specifically hybridizes to a second nucleic acid molecule, the second nucleic acid molecule having a nucleic acid sequence comprising a complement of SEQ ID NO: 51419.

4. A substantially purified protein or fragment thereof encoded by a first nucleic acid molecule according to claim 3, wherein said first nucleic acid molecule comprises a nucleic acid sequence SEQ ID NO: 51419.

5. A purified antibody or fragment thereof which is capable of specifically binding to a protein or fragment thereof, wherein the protein or fragment thereof is encoded by a nucleic acid molecule comprising a nucleic acid sequence SEQ ID NO: 51419

6. A transformed plant having a nucleic acid molecule which comprises: (A) an exogenous promoter region which functions in a plant cell to cause the production of a mRNA molecule; (B) a structural nucleic acid molecule comprising a nucleic acid sequence SEQ ID NO: 51419; and (C) a 3′ non-translated sequence that functions in the plant cell to cause termination of transcription and addition of polyadenylated ribonucleotides to a 3′ end of the mRNA molecule.

7. A transformed plant according to claim 6, wherein said plant is selected from the group consisting of maize and soybean.

8. A transformed plant having a nucleic acid molecule which comprises: (A) an exogenous promoter region which functions in a plant cell to cause the production of a mRNA molecule; which is linked to (B) a transcribed nucleic acid molecule with a transcribed strand and a non-transcribed strand, wherein the transcribed strand is complementary to a nucleic acid molecule comprising a nucleic acid sequence SEQ ID NO: 51419 or fragment thereof; which is linked to (C) a 3′ non-translated sequence that functions in plant cells to cause termination of transcription and addition of polyadenylated ribonucleotides to a 3′ end of the mRNA molecule.

9. A transformed plant according to claim 8, wherein said plant is selected from the group consisting of maize and soybean.

10. A method for determining a level or pattern of a protein in a plant cell or plant tissue under evaluation which comprises assaying the concentration of a molecule, whose concentration is dependent upon the expression of a gene, the gene specifically hybridizes to a nucleic acid molecule having a nucleic acid sequence comprising a complement of SEQ ID NO: 51419, in comparison to the concentration of that molecule present in a reference plant cell or a reference plant tissue with a known level or pattern of the protein, wherein the assayed concentration of the molecule is compared to the assayed concentration of the molecule in the reference plant cell or reference plant tissue with the known level or pattern of the protein.
